(19) United States
(12) Patent Application Publication
(10) Pub. No.: US 2020/0224172 A1
(43) Pub. Date: Jul. 16, 2020

(54) METHODS AND SYSTEMS FOR RECONSTRUCTION OF DEVELOPMENTAL LANDSCAPES BY OPTIMAL TRANSPORT ANALYSIS

(71) Applicants: The Broad Institute, Inc., Cambridge, MA (US); Massachusetts Institute of Technology, Cambridge, MA (US); Whitehead Institute for Biomedical Research, Cambridge, MA (US)

(72) Inventors: Geoffrey Schiebinger, Cambridge, MA (US); Jian Shu, Cambridge, MA (US); Marcin Tabaka, Cambridge, MA (US); Brian Cleary, Cambridge, MA (US); Aviv Regev, Cambridge, MA (US); Eric S. Lander, Cambridge, MA (US); Philippe Rigollet, Cambridge, MA (US)

(21) Appl. No.: 16/648,715

(22) PCT Filed: Sep. 19, 2018
(86) PCT No.: PCT/US18/51808

§ 371 Date: Mar. 19, 2020

Related U.S. Application Data
(60) Provisional application No. 62/560,674, filed on Sep. 19, 2017.
(60) Provisional application No. 62/561,047, filed on Sep. 20, 2017.

(51) Int. Cl. C12N 5/074; C12N 15/86; A61K 35/545; G16B 20/00; G16B 45/00

(52) U.S. Cl. CPC C12N 5/0696; C12N 15/86; A61K 35/545; C12N 2506/1307; G16B 45/00; C12N 2740/15043; C12N 2510/00; G16B 20/00

(57) ABSTRACT

Methods and compositions for producing induced pluripotent stem cell by introducing nucleic acids encoding one or more transcription factors including Obox6 into a target cell.

### CROSS-REFERENCE TO RELATED APPLICATIONS

This application claims the benefit of U.S. Provisional Application Nos. 62/560,674, filed Sep. 19, 2017 and 62/561,047, filed Sep. 20, 2017. The entire contents of the above-identified applications are hereby fully incorporated herein by reference.

### TECHNICAL FIELD

The subject matter disclosed herein is generally directed to methods and systems for analyzing the fates and origins of cells along developmental trajectories using optimal transport analysis of single-cell RNA-seq information over a given time course.

### BACKGROUND

In the mid-20th century, Waddington introduced two images to describe cellular differentiation during development: first, trains moving along branching railroad tracks and, later, marbles following probabilistic trajectories as they roll through a developmental landscape of ridges and valleys (1, 2). These metaphors have powerfully shaped biological thinking in the ensuing decades. The recent advent of massively parallel single-cell RNA sequencing (scRNA-Seq) (3-7) now offers the prospect of empirically reconstructing and studying the actual “landscapes”, “fates” and “trajectories” associated with complex processes of cellular differentiation and de-differentiation—such as organismal development, long-term physiological responses, and induced reprogramming—based on snapshots of expression profiles from heterogeneous cell populations undergoing dynamic transitions (6-11).

To understand such processes in detail, general approaches are needed to answer key questions. For any given system, we would like to know: What classes of cells are present at each stage? For the cells in each class, what was their origin at earlier stages, what are their potential fates at later stages, and what is the actual outcome of a given cell? To what extent are events along a path synchronous or asynchronous? What are the genetic regulatory programs that control each path? What are the intercellular interactions between classes of cells? Answering these questions would provide insights into the nature of developmental processes: How deterministic or stochastic is the process—that is: if, and how early, does it become determined that a particular cell or an entire cell class is destined to a specific fate? For a given origin and target fate, is there only a single path to the target, or are there multiple developmental paths? To what extent is the process cell-intrinsic, driven by intracellular mechanisms that do not require ongoing external inputs, or externally regulated, being affected by other contemporaneous cells? For artificial processes such as induced reprogramming, there are additional questions: What off-target cell classes arise? To what extent do cells activate normal developmental programs vs. unnatural hybrid programs? How can the efficiency of reprogramming be improved?

Experimental approaches to such questions have typically involved studying bulk populations or identifying subsets of cells based on activation of one or a few genes at a specific time (e.g., reporter genes or cell-surface markers) and tracing their subsequent fate. These experiments are severely limited, however, by the need to choose subsets of cells a priori and develop distinct reagents to study each subset. For example, studies of cellular reprogramming from fibroblasts to induced pluripotent cells (iPSCs) have largely relied on RNA- and chromatin-profiling studies of bulk cell populations, together with fate-tracing of cells based on a limited set of markers (e.g., Thy1 and CD44 as markers of the fibroblast state, and ICAM1, Oct4, and Nanog as markers of partial reprogramming) (12-16).

Computational approaches based on single-cell gene expression profiles offer a complementary approach with broader molecular scope, because one can readily define classes of cells based on any expression profile at any stage. The remaining challenge is to reliably infer their trajectories across stages.

Several pioneering papers have introduced methods to infer cellular trajectories (9, 10, 17-29). Early studies recognized that cellular profiles from heterogeneous populations can provide information about the temporal order of asynchronous processes—enabling intermediate transitional cells to be ordered in “pseudotime” along “trajectories”, based on their state of cell differentiation (18). Some approaches relied on k-nearest neighbor graphs (18) or binary trees (9). More recently, diffusion maps have been used to order cell state transitions. In this case, single-cell profiles are assigned to densely populated paths through diffusion map space (20, 21). Each such path is interpreted as a transition between cellular fates, with trajectories determined by curve fitting, and cells “pseudotemporally ordered” based on the diffusion distance to the endpoints of each path. Whereas initial efforts focused mostly on single paths, more recent work has grappled with challenges of branching, which is critical for understanding developmental decisions (10, 11, 21).

While these pioneering approaches have shed important light on various biological systems, many important challenges remain. First, because many methods were initially designed to extract information about stationary processes (such as the cell cycle or adult stem cell differentiation) in which all stages exist simultaneously, they neither directly model nor explicitly leverage the temporal information in a developmental time course (29). Second, a single cell can undergo multiple temporal processes at once. These processes can dramatically impact the performance of these models, with a notable example being the impact of cell proliferation and death (29). Third, many of the methods impose strong structural constraints on the model, such as one-dimensional trajectories and zero-dimensional branch points. This is of particular concern if development follows the flexible “marble” rather than the regimented “tracks” models, in Waddington's frameworks.

### SUMMARY

In one aspect, the present disclosure includes a method of producing induced pluripotent stem cell comprising introducing a nucleic acid encoding Obox6 into a target cell to produce an induced pluripotent stem cell. In some embodiments, the methods further comprises introducing into the target cell at least one nucleic acid encoding a reprogramming factor selected from the group consisting of: Gdf9, Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sal11, Sal14, Esrrb, Nr5a2, Tbx3, and Glis1. In some embodiments, the method further comprises introducing into the target cell at least one nucleic acid encoding a reprogramming factor selected from the group consisting of: Oct4, Klf4, Sox2 and Myc. In some embodiments, the nucleic acid encoding Obox6 is provided in a recombinant vector. In some embodiments, the vector is a lentivirus vector. In some embodiments, the nucleic acid encoding the reprogramming factor is provided in a recombinant vector. In some embodiments, the method further comprises a step of culturing the cells in reprogramming medium. In some embodiments, the method further comprises a step of culturing the cells in the presence of serum. In some embodiments, the method further comprises a step of culturing the cells in the absence of serum. In some embodiments, the induced pluripotent stem cell expresses at least one of a surface marker selected from the group consisting of: Oct4, SOX2, KLf4, c-MYC, LIN28, Nanog, Glis1, TRA-160/TRA-1-81/TRA-2-54, SSEA1, SSEA4, Sal4, and Esrbb1. In some embodiments, the target cell is a mammalian cell. In some embodiments, the target cell is a human cell or a murine cell. In some embodiments, the target cell is a mouse embryonic fibroblast. In some embodiments, the target cell is selected from the group consisting of: fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, pancreatic cells, hepatocytes, macrophages, monocytes, mononuclear cells, and gastric cells, including gastric epithelial cells.

In another aspect, the present disclosure includes a method of producing an induced pluripotent stem cell comprising introducing at least one of Obox6, Spic, Zfp42, Sox2, Mybl2, Msc, Nanog, Hesx1 and Esrrb into a target cell to produce an induced pluripotent stem cell.

In another aspect, the present disclosure includes a method of producing an induced pluripotent stem cell comprising introducing at least one of the transcription factors identified in Table 2, Table 3, Table 4, Table 5, and Table 6, into a target cell to produce an induced pluripotent stem cell.

In another aspect, the present disclosure includes a method of increasing the efficiency of production of an induced pluripotent stem cell comprising introducing Obox6 into a target cell to produce an induced pluripotent stem cell.

In another aspect, the present disclosure includes a method of increasing the efficiency of production of an induced pluripotent stem cell comprising introducing at least one of the transcription factors identified in Table 2, Table 3, Table 4, Table 5, and Table 6, into a target cell to produce an induced pluripotent stem cell.

In another aspect, the present disclosure includes an isolated induced pluripotential stem cell produced by the methods disclosed herein.

In another aspect, the present disclosure includes a method of treating a subject with a disease comprising administering to the subject a cell produced by differentiation of the induced pluripotent stem cell produced by the methods disclosed herein.

In another aspect, the present disclosure includes a composition for producing an induced pluripotent stem cell comprising Obox6 in combination with reprogramming medium.

In another aspect, the present disclosure includes a composition for producing an induced pluripotent stem cell comprising one or more of the factors identified in or one or more of the factors identified in Table 2, Table 3, Table 4, Table 5, and Table 6 in combination with reprogramming medium.

In another aspect, the present disclosure includes use of Obox6 for production of an induced pluripotent stem cell.

In another aspect, the present disclosure includes use of a factor identified in or one or more of the factors identified in Table 2, Table 3, Table 4, Table 5, and Table 6 for production of an induced pluripotent stem cell.

In another aspect, the present disclosure includes a method of increasing the efficiency of reprogramming a cell comprising introducing Obox6 into a target cell to produce an induced pluripotent stem cell.

In another aspect, the present disclosure includes a method of increasing the efficiency of reprogramming a cell comprising introducing at least one of the transcription factors identified in Table 2, Table 3, Table 4, Table 5 and Table 6, into a target cell to produce an induced pluripotent stem cell.

In another aspect, the present disclosure includes a computer-implemented method for mapping developmental trajectories of cells, comprising: generating, using one or more computing devices, optimal transport maps for a set of cells from single cell sequencing data obtained over a defined time course; determining, using one or more computing devices, cell regulatory models, and optionally identifying local biomarker enrichment, based on at least the generated optimal transport maps; defining, using the one or more computing devices, gene modules; and generating, using the one or more computing devices, a visualization of a developmental landscape of the set of cells.

In some embodiments, determining cell regulatory models comprise sampling pairs of cells at a first time and a second time point according to transport probabilities. In some embodiments, the method further comprises using the expression levels of transcription factors at the earlier time point to predict non-transcription factor expression at the second time point. In some embodiments, identifying local biomarker enrichment comprises identifying transcription factors enriched in cells having a defined percentage of descendants in a target cell population. In some embodiments, the defined percentage is at least 50% of mass. In some embodiments, defining gene modules comprises partitioning genes based on correlated gene expression across cells and clusters. In some embodiments, partitioning comprises partitioning cells based on graph clustering. In some embodiments, graph clustering further comprises dimensionality reduction using diffusion maps. In some embodiments, the visualization of the developmental landscape comprises high-dimensional gene expression data in two dimensions. In some embodiments, the visualization is generated using force-directed layout embedding (FLE). In some embodiments, the visualization provides one or more cell types, cell ancestors, cell descendants, cell trajectories, gene modules, and cell clusters from the single cell sequencing data.

In another aspect, the present disclosure includes a computer program product, comprising: a non-transitory computer-executable storage device having computer-readable program instructions embodied thereon that when executed by a computer cause the computer to execute the methods disclosed herein.

In another aspect, the present disclosure includes a system comprising: a storage device; and a processor communicatively coupled to the storage device, wherein the processor executes application code instructions that are stored in the storage device and that cause the system to executed the methods disclosed herein.

In another aspect, the present disclosure includes a method of producing an induced pluripotent stem cell comprising introducing a nucleic acid encoding Gdf9 into a target cell to produce an induced pluripotent stem cell.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—is a block diagram depicting a system for mapping developmental trajectories of cells, in accordance with certain example embodiments

FIG. 2—is a block flow diagram depicting a method for mapping development trajectories of cells, in accordance with certain example embodiments.

FIG. 3—is a diagram showing data Sᵢ from a generic branching developmental process. The x-axis represents the time and the y-axis represents expression.

FIG. 4—provides a schematic of a regulatory vector file which gives rise to a time-dependent probability distribution.

FIGS. 5A-5G—(FIGS. 5A-5B) Waddington's classical analogies of cells undergoing differentiation, initially (1936) illustrated by railroad cars on switching tracks (FIG. 5A) and later (1957) by marbles rolling in a landscape (FIG. 5B), with trajectories shaped by hills and valleys. (FIGS. 5C-E) Differentiation processes in which the ultimate fate of individual cells (filled dots) is (C) predetermined (FIG. 5D) not predetermined, or (FIG. 5E) progressively determined. Arrows indicate possible transitions, and color represents cell fate, with red and blue indicating distinct fates, light red and light blue indicating partially determined fates, and grey indicating undetermined fate. (FIG. 5F) Illustration of transported mass. A transport map, describes how a point x at one stage (X) is redistributed across all points (denoted by “ ”) at the subsequent stage (Y). (FIG. 5G) Transport maps computed from a time series of samples taken from a time-varying distribution. Between each pair of time points, a transport map redistributes the cells observed at time to match the distribution of cells observed at time.

FIGS. 6A-6C—(FIG. 6A) Representation of reprogramming procedure and time points of sample collection. (Top) Mouse embryos (E13.5) were dissected to obtain secondary MEFs (2° MEF), which were reprogrammed into iPSCs. In Phase-1 of reprogramming (light blue; days 0-8), doxycycline (Dox) was added to the media to induce ectopic expression of reprogramming factors (Oct4, Klf4, Sox2, and Myc). In Phase-2 (days 9-16), Dox was withdrawn from the media, and cells were grown either in the presence of 2i (light red) or serum (light green). Samples were also collected from established iPSC lines reprogrammed from the same 2° MEFs, maintained in either 2i or serum conditions (far right in each time course). Individual dots along the time course indicate time points of scRNA-Seq collection, with two dots indicating biological replicates. (FIG. 6B) Number of scRNA-Seq profiles from each sample collection that passed quality control filters. (FIG. 6C) Bright field images of day 0 (Phase1-(Dox)) and day 16 cells during reprogramming in (Phase-2(2i)) and (Phase-2(serum)) culture conditions.

FIGS. 7A-7F—scRNA-Seq profiles of all 65,781 cells were embedded in two-dimensional space using FLE, and annotated with indicated features. (FIG. 7A) Unannotated layout of all cells. Each dot represents one cell. (FIGS. 7B-7C) Annotation by time point (color) and biological feature, with Phase-2 points from either (FIG. 7B) 2i condition or (FIG. 7C) serum condition. Phase-1 points appear in both (FIG. 7B) and (FIG. 7C). Individual cells are colored by day of collection, with grey points (BC, background color) representing Phase-2 cells from serum (in FIG. 7B) or 2i (in FIG. 7C). (FIG. 7D) Annotation by cell cluster. Cells were clustered on the basis of similarity in gene expression. Each cell is colored by cluster membership (with clusters numbered 1-33). (FIGS. 7E-7F) Annotation by gene signature (FIG. 7E) and individual gene expression levels (FIG. 7F). Individual cells are colored by gene signature scores (in FIG. 7E) or normalized expression levels (in FIG. 7F; where E is the number of transcripts of a gene per 10,000 total transcripts).

FIGS. 8A-8F—(FIG. 8A) Schematic representation of the major cluster-to-cluster transitions (see Table 10 for details[BC17]). Individual arrows indicate transport from ancestral clusters to descendant clusters, with colors corresponding to the ancestral cluster. For each descendant cluster, arrows were drawn when at least 20% of the ancestral cells (at the previous time point) were contained within a given cluster (self-loops not shown). Arrow thickness indicates the proportion of ancestors arising from a given cluster. (FIG. 8B) Heatmap depiction of cluster descendants in 2i condition. In each row of the heatmap, color intensity indicates the number of descendant cells (“mass”, normalized to a starting population of 100 cells) transported to each cluster at the subsequent time point (see Table 10 for details). Clusters with highly-proliferative cells (e.g., cluster 4) transport more total mass than clusters with lowly-proliferative cells (e.g., cluster 14). ((FIG. 8C) Depiction of divergent day 8 descendant distributions for two clusters of cells at day 2 (cluster 4 (left) and cluster 6 (right). Color intensity indicates the distribution of descendants at day 8, with bright teal indicating high probability fates and gray indicating low probability fates. (FIG. 8D) Enrichment of the ancestral distributions of iPSCs, Valley of Stress, and alternative fates (neuron-like and placenta-like) in clusters of day 2 cells. The red horizontal dashed line indicates a null-enrichment, where a cluster contributes to the ancestral distribution in proportion to its size. Cluster 4 has a net positive enrichment because its descendants are highly proliferative, while cluster 6 has a net negative enrichment because its descendants are lowly proliferative. (FIG. 8E) and (FIG. 8F) Ancestral trajectories of indicated populations of cells at day 16 (iPSCs, placental, neural-like cells, etc.) in serum (FIG. 8E) and 2i (FIG. 8F). Clusters used to define the indicated populations are shown in parentheses. Colors indicate time point. Sizes of points and intensity of colors indicate ancestral distribution probabilities by day (color bars, right; BC, background color, representing cells from the other culture condition).

FIGS. 9A-9D—(FIG. 9A) Classification of genes into 14 groups based on similar temporal expression profiles along the trajectory to successful reprogramming. Averaged gene expression profiles for each group, in 2i and serum conditions (left). Heatmap for genes within each group, with intensity of color indicating log 2-fold change in expression relative to day 0 (middle). Representative genes and top terms from gene-set enrichment analysis for each group (right). (FIG. 9B) Comparison of FACS and in silico sorting experiments. Scatterplot shows reprogramming efficiencies determined by FACS sort and growth experiments (blue triangles) (16) and our computationally inferred trajectories (red squares). The specific cell surface markers used for the in silico and experimental methods are indicated. Reprogramming efficiencies for these categories (calculated both experimentally and in silico) are normalized to the percentage of EGFP+ colonies in CD44⁻ICAM1⁺Nanog⁺condition (details found in Appendix 5). (FIG. 9C) Schematic of regulatory model in which TF expression in ancestral cells is predictive of gene expression in descendant cells. (FIG. 9D) Onset of iPSC-associated TFs in 2i (left) and serum (right). (Top) Mean expression levels weighted by iPSC ancestral distribution probabilities (Y axis) of Nanog, Obox6, and Sox2 at each day (X axis). (Bottom) Normalized expression of TF modules “A” and “B” from our regulatory model (as in FIG. 9B) that were associated with gene expression in iPSCs.

FIGS. 10A-10C—(FIGS. 10A-10B) Bright field and fluorescence images of iPSC colonies generated by lentiviral overexpression of Oct4, Klf4, Sox2, and Myc (OKSM) with either an empty control, Zfp42 or Obox6 expression cassette, in either Phase-1(Dox)/Phase-2(2i) (FIG. 10A) and Phase-1(Dox)/Phase-2(serum) (FIG. 10B) conditions (indicated). Cells were imaged at day 16 to measure Oct4-EGFP⁺cells. Bar plots representing average percentage of Oct4-EGFP⁺colonies in each condition on day 16 are included below the images. Shown are data from one of five independent experiments, with three biological replicates each. Error bars represent standard deviation for the three biological replicates. (FIG. 10C) Schematic of the overall reprogramming landscape highlighting: the progression of the successful reprogramming trajectory, alternative cell lineages, and specific transition states (Horn of Transformation). Also highlighted are transcription factors (orange) predicted to play a role in the induction and maintenance of indicated cellular states, and putative cell-cell interactions between contemporaneous cells in the reprogramming system.

FIGS. 11A-11D—Single-cell RNA-Seq quality metrics. (FIG. 11A) Correlation between number of genes and tran-scripts per cell (log 10 transformed). Cells with fewer than 1000 genes detected were filtered out. The color gradient represents cell density. (FIG. 11B) Variation in single cell data depicted by correlation between transcript levels (log 10 transformed average transcript counts) detected in biological replicates generated from day 10 samples in 2i conditions. Pearson correlation coefficient (r) is given. The color gradient represents cell density. (FIG. 11C) Biological variation in single cell data depicted by correlation between tran-script levels (log 10 transformed average transcript counts) detected in iPSCs and MEFs. Pearson correlation coefficient (r) is given. The color gradient represents cell density. (FIG. 11D) Correlogram visualizing correlation between single cell gene expression profiles between various time points and their biological replicates. In this plot, the correlation coefficients (circles) are colored according to their values, ranging from 0.75 (blue) to 1 (red). The size of the circles represents the magnitude of the coefficient. The replicates within the timepoints are denoted with suffixes 1 and 2.

FIGS. 12A-12C—Comparison of various dimensionality reduction methods to visualize single cell RNA-Seq data. High-dimensional structure of single-cell expression data was embedded in low-dimensional space for visualization using (FIG. 12A) the Force-directed Layout Embedding algorithm (FLE) (directed graph approach) and the t-Distributed Stochastic Neighbor Embedding algorithm (t-SNE) with (FIG. 12B) principal components and (FIG. 12C) diffusion maps as input parameters.

FIG. 13—Visualization of gene modules across reprogramming time points. Expression profiles of all 65,781 cells studied were embedded in two-dimensional space, using force-directed layout embed-ding (FLE). The layouts were annotated by single-cell z-scores for 44 gene modules (details in Table 1). The color gradient represents the distribution of z-scores across all cells for a given gene module.

FIGS. 14A-14B—Characterization of cell clusters. (FIG. 14A) Heatmap representing the enrichment of cells from the indicated samples at various time points and culture conditions across 33 different clusters. The color gradient represents the range of cell fractions from 0-0.25. (FIG. 14B) Heatmap depicting the enrichment of correlated gene modules within specific cell clusters. The color gradient represents the average gene module scores at the indicated cell clusters. Specific cell clusters that show highly correlated gene module scores were numerically labeled as shown

FIG. 15—Visualization of individual gene expression levels. Normalized expression levels [log 2(E+1)] for indicated genes were used to annotate force-directed layout embedding (FLE) graphs generated from the expression profiles of 65,781 cells. E represents the number of transcripts of a gene per 10,000 total transcripts

FIGS. 16A-16E—Distribution of gene signatures. (FIG. 16A) Distribution of proliferation scores for cells at day 0 (solid black). Proliferation scores were calculated from combined expression levels of G1/S and G2/M cell cycle genes (see Appendix 5). Normal mixture modeling (dashed line) was used to classify the cells based on proliferation scores into non-cycling (red) and cycling (blue) cells (top). Visualization of the cycling and non-cycling of cells on FLE at day 0 (bottom). (FIG. 16B) Violin plots of single-cell scores for indicated gene signatures and Shisa8 expression levels in clusters 3, 4, 5, and 6. (FIG. 16C) Violin plots of single cell scores for indicated gene signatures in clusters 7, 8, and 18. (FIG. 16D) Bar plots of normalized expression levels [log 2(E+1)] for indicated genes, where E is the number of transcripts of a gene per 10,000 total transcripts. (FIG. 16E) Single-cell scores for indicated gene signatures across all 33 cell clusters.

FIGS. 17A-17C—Heatmap depiction of origins and fates of cells inferred from optimal transport. Heatmap depiction of cluster descendants in (FIG. 17A) serum condition, and cluster ancestors in (FIG. 17B) 2i and (FIG. 17C) serum conditions. Each row of the heatmap in (FIG. 17A) shows how the descendants of the cells in a particular cluster are distributed over all clusters. Color intensity indicates the number of descendant cells (“mass”, normalized to a starting population of 100 cells) transported to each cluster at the next time point. Each column of the heatmaps in (FIG. 17B, FIG. 17C) shows how the ancestors of a particular cluster are distributed over all clusters. Table 10 contains the specific numerical values.

FIGS. 18A-18F—Potential cell-cell interactions across the reprogramming time course. (FIG. 18A) Temporal pattern of the net potential for paracrine signaling between contemporaneous cells. Each dot represents the aggregated interaction score across all ligand-receptor pairs for a given combination of clusters (all 149 detected ligands). The aggregate interaction score is defined as a sum of individual interaction scores. (FIG. 18B) As in A, but genes specific to SASP signature are considered (20 detected ligands). (FIG. 18C) Heatmap representing the aggregate interaction scores on day 16 cells in 2i condition for ligands specific to SASP signature. Rows correspond to clusters of cells expressing ligands. Columns correspond to clusters of cells expressing cognate receptors. Only clusters containing more than 1% of cells from day 16 (2i) are shown. (FIGS. 18D-18F) Potential ligand-receptor pairs ranked by their standardized interaction scores calculated from the permuted data (see Appendix 5 for details). Ligand-receptor pairs between (FIG. 18D) valley of stress cells (clusters 11-17) and iPSCs (clusters 28-33) on day 16 (2i), (FIG. 18E) valley of stress cells and preneural/neural-like cells (clusters 23, 26, and 27) on day 16 (serum), and (FIG. 18F) placental-like cells (clusters 24 and 25) and valley of stress cells on day 12 (2i)

FIGS. 19A-19F—Gene modules and associated transcription factors based on optimal transport. Using optimal transport trajectories, TF levels in cells at time t are used to predict the activity levels of gene modules in descendant cells at time t+1. Gene modules are learned during model training to capture coherent expression programs. For five modules (FIGS. 19A-19E), bar plots depict the top 50 genes in the module (black), and the top 20 TFs each associated with positive (red) and negative (blue) module activity. (FIGS. 19A-19B) Two modules that are active in cells with placental identity. (FIG. 19C) A module active in cells with neural identity. (FIG. 19D-19E) Two modules active in successfully reprogrammed cells. (FIG. 19F) Enrichment analysis of TFs in day 12 cells with high (>80%) vs. low (<20%) probability of successful reprogramming. Dot size and color represent percentage of day 12 cells expressing the indicated TF in high- or low-probability cells. Bar heights indicate the fold enrichment in high-vs. low-probability cells.

FIGS. 20A-20C—Effect of overexpression of Obox6 and Zpf42 on reprogramming efficiency. (FIG. 20A) Percentage of Oct4-EGFP+ cells at day 16 of reprogramming from secondary MEFs by lentiviral overexpression of Oct4, Klf4, Sox2, and Myc (OKSM) combined with either Zfp42, Obox6, or an empty control, in either 2i or serum conditions. Oct4-EGFP+ cells were measured by flow cytometry. Plot includes the percentage of Oct4-EGFP+ cells in three biological replicates (for Zfp42 and Obox6 overexpression, or an empty control) from five independent experiments (Exp). (FIG. 20B, FIG. 20C) Number of Oct4-EGFP+ colonies at day 16 of reprogramming from primary MEFs by lentiviral overexpression of individual Oct4, Klf4, Sox2, and Myc combined with either Zfp42, Obox6, or an empty control in (FIG. 20B) 2i and (FIG. 20C) serum conditions. Plot includes the number of Oct4-EGFP+ cells in three biological replicates (for Zfp42 and Obox6 overexpression, or an empty control) from two independent experiments (Exp).

FIGS. 21A-21E—X-chromosome reactivation. (FIGS. 21A-21C) Boxplots showing X/Autosome expression ratio (left panel) and Xist expression log 2(E+1) across individual cells by clusters (right panel): (FIG. 21A) all cells, (FIG. 21B) phase-1(Dox) and phase-2(2i) cells, (FIG. 21C) phase-1(Dox) and phase-2(serum) cells. (FIGS. 21D-21F)—X/Autosome expression ratio and A6, A7 activation pattern changes along the successful trajectory determined by optimal transport: Relative gene expression changes of individual genes from A6 (FIG. 21D) and A7 (FIG. 21E) activation patterns (gray solid lines). Black and blue solid lines correspond to average relative expression of genes and average X/Autosome expression ratios, respectively. (FIG. 21F) Comparison between activation of A6 and A7 programs (average relative expression) with X/Autosome expression ratio. Distribution of X/Autosome expression ratios (FIG. 21G) and A7 scores (FIG. 21H) across all cells. Dotted lines represent threshold values used in classification of cells that reactivated X-chromosome (>1.4) and upregulated A7 genes (>0.25).

FIGS. 22A-22C—Single-cell expression levels were used to identify cells with aberrant expression in large chromosomal regions. (FIG. 22A) Whole chromosome aberrations were detected in 1% of all cells. Each dot represents one chromosome (X axis) in a single cell with significant aberrations (FDR 10%), with violin plots capturing the distributions of dots. The net expression of these chromosomes relative to the average expression across all cells (Y axis) is 1.7-fold higher (median, left panel) and 2.2-fold lower (right panel), indicating whole chromosome gain and loss, respectively. The median relative expression levels are slightly higher (lower) than the 1.5-fold (2-fold) increase (decrease) that would be expected from a true chromosomal gain (loss) because our statistics are conservative in calling significant events but allow for a long tail of high (low) expression. (FIG. 22B) Visualization of cells with significant subchromosomal aberrations (red) in FLE. (FIG. 22C) Bar plots depict the fraction of cells in each cluster with significant subchromosomal (25-200 Mbp) aberrations (FDR 10%).

FIGS. 23A-23F—Modeling developmental processes with optimal transport. Waddington-OT: a probabilistic model for developmental processes. (FIG. 23A) A temporal progression of a time-varying distribution [symbol] ₜ (left) can be sampled to obtain finite empirical distributions of cells [symbol] ₜ_{ᵢ} at various time points t₁, t₂, t₃ (right). Over short time scales, the unknown true coupling, γₜ_{₁}_{,t}_{₂}, is assumed to be close to the optimal transport coupling, πₜ_{₁}_{,t}_{₂}, which can be approximated by πₜ_{₁}_{,t}_{₂} computed from the empirical distributions [symbol] ₜ_{₁} and [symbol] ₜ_{₂}. (FIGS. 23B-23F) Simulated data and analysis performed by Waddington-OT. (FIG. 23B) Single-cell profiles (individual dots) are embedded in two dimensions and colored by the time of collection. Optimal transport can be used to calculate the descendant trajectories (FIG. 23C) and ancestor trajectories (FIG. 23D) of any subpopulation of interest (cells highlighted in black; color indicates time). Ancestor distributions of distinct subpopulations can be compared to calculate their shared ancestry (FIG. 23E) (ancestors of each population shown in red and blue, shared ancestors in purple). (FIG. 23F) The expression of gene signatures (left; green, high expression; grey, low expression) can be predicted from the earlier expression of transcription factors (middle; black, high expression; grey, low expression) in a gene regulatory model by analyzing trends along ancestor trajectories. In the plot at right, at each time point, the height of the curve depicts the average expression in the ancestors of cells in the leftmost tip.

FIGS. 24A-24H—A single cell RNA-Seq time course of iPSC reprogramming. (FIG. 24A) Representation of reprogramming procedure and time points of sample collection. (Top) Mouse embryos (E13.5) were dissected to obtain secondary MEFs (2° MEF), which were reprogrammed into iPSCs. In Phase-1 of reprogramming (light blue; days 0-8), doxycycline (Dox) was added to the media to induce ectopic expression of reprogramming factors (Oct4, Klf4, Sox2, and Myc). In Phase-2 (days 9-18), Dox was withdrawn from the media, and cells were grown either in the presence of 2i (light red) or serum (light green). Samples were also collected from established iPSC lines reprogrammed from the same 2° MEFs, maintained in either 2i or serum conditions (far right in each time course). Individual dots indicate time points of scRNA-Seq collection. (FIGS. 24B-24E) scRNA-Seq profiles of all 251,203 cells (individual dots) were embedded in two-dimensional space using FLE, and annotated with indicated features. (FIG. 24B) Unannotated layout of all cells, with the density of cells in each region indicated by intensity. (FIG. 24C) Cells colored by time point, with Phase-2 points from either 2i condition (left) or serum condition (right). Phase-1 points appear in both subplots. Grey points represent Phase-2 cells from the other condition. (FIG. 24D) In different regions of the FLE, cells have distinct expression patterns of six major gene signatures (average expression z-score of genes in a signature indicated by red color bar). Gene signature activity and trajectory analysis were used to define the major cell sets (FIG. 24E) and to establish the overall flow through the landscape (FIG. 24F) (schematic representation). (FIG. 24G) The relative abundance (y-axis) of each cell set (colored lines) is plotted over time (x-axis) in 2i (top) and serum (bottom). (FIG. 24H) Validation via geodesic interpolation in serum condition. Data at withheld timepoints (x-axis) are interpolated using data at the neighboring timepoints. Interpolation is done using a null estimator of independent coupling (blue) and the optimal transport coupling (red), with the distance between interpolated and withheld data indicated on the y-axis. The distance between two batches of withheld data at the same point is shown in green. Shaded regions indicate standard deviations over independent samples of the coupling map.

FIGS. 25A-25H—In initial stages of reprogramming, cells progress toward stromal or MET fates. (FIG. 25A) Cells in the stromal region have higher expression of gene signatures (red color bar, average z-score) and individual genes (red color bar, log(TPM+1)) that are associated with stromal activity and senescence. Ancestors of day 18 stromal cells are visualized on the FLE (FIG. 25B) (colored by day, intensity indicates probability), and expression trends along this ancestor trajectory (FIG. 25C) are depicted for gene signatures (left) and individual transcription factors (TFs; right). The ancestors of day 8 MET cells (FIG. 25D) have a distinct trajectory and gene signature trends (FIG. 25E), and show differential expression of several TFs (FIG. 25F) (dashed line, average TPM in stromal ancestors; solid line, average TPM in MET ancestors). (FIG. 25G, FIG. 2511) The MET and stromal fates are gradually specified from day 0 through 8. Color bar in (FIG. 25G) indicates log-likelihood of obtaining stromal vs. MET fate. (FIG. 2511) The extent to which the stromal ancestor distribution has diverged (y-axis) from all other fates at each point in time (x-axis). The divergence is quantified as ½ times the total variation distance between the ancestor distributions.

FIGS. 26A-26F—iPSCs emerge from cells in the MET Region. (FIG. 26A) Ancestors of day 18 iPSCs in 2i (left) and serum (right) are visualized on the FLE (colored by day, intensity indicates probability). Cells in the iPSC region express pluripotency marker genes (FIG. 26B) (red color bar, log(TPM+1)) and diverge from alternative fates also arising from the MET region (neural, epithelial, and trophoblast) from days 8-12 (FIG. 26C) (divergence between pairs of lineages indicated by individual lines; green line, divergence between iPSC and all others). (FIG. 26D) Expression trends along the ancestor trajectory in serum are depicted for gene signatures (left) and individual transcription factors (right). (FIG. 26E) A signature of X reactivation (left; red color bar, average z-score) and Xist expression (right; log(TPM+1)) visualized on the FLE. (FIG. 26F) Trends in X-inactivation, X-reactivation and pluripotency along the iPSC trajectory in 2i. The values on the axis refer to average expression across early (black) and late (red) pluripotency activation genes, Xist average expression (log(TPM+1), orange) and X/Autosome expression ratio (blue) along the iPSC trajectory.

FIGS. 27A-27G—Extra-embryonic and neural-like cells emerge during reprogramming. Subpopulations of trophoblast—(FIGS. 27A-27C) and neural-like (FIGS. 27D-27G) cells are found in the late stages of reprogramming. Ancestors of day 18 trophoblasts are visualized on the FLE (FIG. 27A) (colored by day, intensity indicates probability), and expression trends along the ancestor trajectory in serum (FIG. 27B) are depicted for gene signatures (left) and individual transcription factors (right). (FIG. 27C) Cells in the trophoblast cell set were re-embedded by FLE, and scored for signatures of trophoblast progenitors (TP), spiral artery trophoblast giant cells (SpA-TGC), and spongiotrophoblasts (SpTB). Colors indicate significant expression of TP, SpA-TGC, and SpTB signatures (−log 10(FDR q-value)), or expression of labyrinthine trophoblast marker gene Gcm1 (red color bar, log(TPM+1)). Ancestors of day 18 cells in the neural region are visualized on the FLE (FIG. 27D) (colored by day, intensity indicates probability), and expression trends along the ancestor trajectory in serum (FIG. 27E) are depicted for gene signatures (left) and individual transcription factors (right). (FIG. 27F) Cells with radial glial (RG) and differentiated subtype signatures begin to appear around day 12 (x-axis, time; y-axis, relative abundance in serum). (FIG. 27G) All cells in the neural region we re-embedded by FLE, and scored for significant expression of differentiated signatures (OPC, astrocyte, cortical neurons; color, −log 10(FDR q-value)), or annotated by expression of markers of inhibitory and excitatory neurons (red color bars, log(TPM+1)). OPC, oligodendrocyte precursor cells.

FIGS. 28A-28K—Paracrine signaling and genomic aberrations. (FIG. 28A) Schematic of the paracrine signaling interaction scores. High potential interaction occurs between two groups of contemporaneous cells in which one group secretes a ligand and a second group expresses a cognate receptor. (FIG. 28B) Temporal pattern of the net potential for paracrine signaling between contemporaneous cells in serum condition. Each dot represents the aggregated interaction score across all ligand-receptor pairs for a given combination of clusters (FIG. S5A, all 180 detected ligands). The aggregate interaction score is defined as a sum of individual interaction scores. (FIGS. 28C-E) Potential ligand-receptor pairs between ancestors of stromal cells and iPSCs (FIG. 28C), neural-like cells (FIG. 28D), and trophoblasts (FIG. 28E), ranked by their standardized interaction scores calculated from the permuted data (see STAR Methods for details). (FIGS. 28F-H) Individual cells on the FLE colored by the expression level (log(TPM+1)) of ligands (upper row) and receptors (lower row) for top interacting pairs between stromal cells and iPSCs (FIG. 28F), neural-like cells (FIG. 28G), and trophoblasts (FIG. 2811). (FIGS. 28I-28K) Evidence for genomic aberrations was found at the level of whole chromosomes (I) and sub-chromosomal regions spanning 25 housekeeping genes (FIGS. 28J, 28K). (FIG. 28I) Average expression of housekeeping genes on chromosomes (numbered on x-axis) in single cells (dots with violin plots) with evidence of genomic amplification (left panel) or loss (right panel), relative to all cells without evidence of aberrations (y-axis, relative expression). (FIG. 28J) Individual cells on the FLE are colored by statistical significance (−log 10(q-value), colorbar) of evidence for sub-chromosomal aberrations. (FIG. 28K) Average expression of genes on chromosome 15 in trophoblast-like cells with evidence of a recurrent sub-chromosomal amplification (FDR 10%, region indicated by red lines), relative to trophoblast-like cells without evidence of amplification in this region (y-axis, relative expression).

FIGS. 29A-29D—Obox6 enhances reprogramming. (FIG. 29A) For cells (individual dots) at each timepoint (x-axis), the log-likelihood ratio of obtaining iPSCs fate vs non iPSCs fate in 2i is depicted on the y-axis. Cells expressing Obox6 are highlighted in red. (FIG. 29B) Bright field and fluorescence images of iPSC colonies generated by lentiviral overexpression of Oct4, Klf4, Sox2, and Myc (OKSM) with either an empty control, Zfp42 or Obox6 expression cassette, in Phase-1(Dox)/Phase-2(2i). (FIG. 29C) Bar plots representing average percentage of Oct4-EGFP⁺colonies in 2i on day 16. Data shown is one of five independent experiments, with three biological replicates each. Error bars represent standard deviation for the three biological replicates. (FIG. 29D) Schematic of the overall reprogramming landscape in serum highlighting: the progression of the successful reprogramming trajectory (represented in black), alternative cell lineages and subtypes within these lineages (Stromal in blue, trophoblast-like in red, neural in green and epithelial in orange), and specific transition states (MET in purple). Also highlighted are transcription factors predicted to play a role in the transition to indicated cellular states (as indicated by the specific color), and putative cell-cell interactions between contemporaneous cells in the reprogramming system. i and e Neurons refers to inhibitory and excitatory neurons respectively.

FIGS. 30A-30G—Related to FIGS. 24A-24H: Validation, stability, and comparison to pilot study. (FIGS. 30A-30C) Unbalanced transport can be used to tune growth rates. (FIG. 30A) When the unbalanced regularization parameter is large (=16), growth constraints are imposed strictly, and the input growth (x-axis; determined by gene signatures—see STAR Methods) is well-correlated to the output growth (y-axis; implicit growth rate determined from the transport map). (FIG. 30B) When the unbalanced parameter is small (=1), the growth constraints are only loosely imposed, allowing implicit growth rates to adjust and better fit the data. (FIG. 30C) The correlation of output vs input growth as a function of. (FIG. 30D) Validation by geodesic interpolation for 2i conditions. As in FIG. 24H (which shows serum), the red curve shows the performance of interpolating held-out time points with optimal transport. The green curve shows the batch-to-batch Wasserstein distance for the held-out time points, which is a measure of the baseline noise level. The blue curve shows the performance of a null model (interpolating according to the independent coupling, including growth). (FIGS. 30E-30F) Comparison to pilot dataset. (FIG. 30E) Trends in signature scores along ancestor trajectories to iPSC, Stromal, Neural, and Trophoblast cell sets. Trends for the pilot dataset are shown with open circles and trends for the large dataset are shown with solid lines. (FIG. 30F) Shared ancestry results for pilot dataset (solid lines) and for the larger dataset (dashed lines). (FIG. 30G) Bright field images of day 2 (Phase1-(Dox)), day 4 (Phase1-(dox)) and day 18 cells during reprogramming in (Phase-2(2i)) and (Phase-2(serum)) culture conditions. BF (bright field). GFP (Oct4-GFP).

FIGS. 31A-31F—Related to FIGS. 25A-25H Divergence of Stromal and MET fates during the initial stages of reprogramming. (FIGS. 31A-31B) Cells from the stromal region were re-embedded by FLE, and scored for signatures of long-term cultured MEFs (left) or stromal cells in the embryonic mesenchyme (right) found in the Mouse Cell Atlas (FIG. 31A), or from signatures derived from genes co-expressed (see STAR-Methods) with Cxcl12, Ifitm1, or Matn4 in the stromal cell set (FIG. 31B) (red color bars, average z-score of expression). (FIG. 31C) Ectopic OKSM expression levels are predictive of MET fate. The y-axis shows correlation between OKSM expression and the log-likelihood of obtaining MET fate. Color (red vs blue) distinguishes the two batches at each time point (x-axis). (FIG. 31D) Fut9+ and Shisa8+ expression patterns visualized in a fate-divergence layout. Each dot represents a single cell, colored by expression of either Fut9 (left) or Shisa8 (right). The x-axis shows time of collection and the y-axis shows the log-likelihood ratio of obtaining MET vs Stromal fate, as predicted by optimal transport. (FIG. 31E) The Stromal region is a terminal destination as evidenced by (1) the large flow of cells into the region around day 9 (green spike, first and second panels) and (2) essentially zero flow out of the region (blue curves, first and second panels). By contrast, the MET region is a transient state as evidenced by the blue curves in the right two panels showing significant transitions out of MET. (FIG. 31F) Day 0 MEFs (DO; black dots) we re-embedded together with cells from the stromal set (red dots) in a TSNE plot.

FIGS. 32A-32C—Related to FIGS. 26A-26F: iPSCs. (FIG. 32A) Cells with significant expression of 2 cell (2C), 4 cell (4C), 8 cell (8C), 16 cell (16C) and 32cell (32C) signatures at an FDR of 10% on iPSC-specific FLE. (FIG. 32B) Overlap between different early embryonic stages. The horizontal bars show the number of cells identified as 2C, 4C, 8C, 16C, or 32C. The vertical bars indicate the number of cells in each possible combination of these cell sets (e.g. 2C and 4C). (FIG. 32C) Heatmap showing trends in expression of 1479 variable genes (STAR-Methods) along the ancestor trajectory to iPSCs. Color indicates fold-change in expression relative to day 0 (white). Each row shows the mean expression trend for a single gene, where the mean is computed with respect to the ancestor distribution. Genes are clustered into groups with similar trends. Terms on the right indicate significant gene set enrichment (GSEA, all adjusted p-values<0.01) in one of several databases (M, MSigDB; BP, GO biological process; W, WikiPathways; C, chromosome; CC, GO cellular component).

FIGS. 33A-33E—Related to FIGS. 27A-27G: Trophoblast and Neural subtypes. (FIG. 33A) Expression of individual marker genes (red color bars, log(TPM+1); see also Table S2) for each subtype on the trophoblast FLE (as in FIG. 5C). TP, trophoblast progenitors; SpA-TGC, spiral artery trophoblast giant cells; SpTB, spongiotrophoblasts; LaTB, labyrinthine trophoblasts. (FIG. 33B) Cells with a gene signature of extra-embryonic endoderm (XEN) arise in a single batch on day 15.5 (red color bar, average z-score). (FIGS. 33C-33E) Cells in the neural region were re-embedded by tSNE and annotated with various features. (FIG. 33C) Marker gene expression (red color bar, log(TPM+1)) of neural subtypes on the neural tSNE. (FIG. 33D) Cells with significant expression (black dots) of indicated signatures from the Allen Mouse Brain Atlas on the neural tSNE at an FDR of 10%. OPC refers to oligodendrocyte precursor cells. (FIG. 33E) Cells in the neural region present from days 12.5-14.5 (left) or days 17-18 (right).

FIGS. 34A-34E—Related to FIGS. 28A-28K: Temporal patterns of paracrine signaling. (FIG. 34A) Cell clusters determined by Louvain-Jaccard community detection algorithm. (FIG. 34B) Temporal pattern of the net potential for paracrine signaling between contemporaneous cells in 2i condition. Each dot represents the aggregated interaction score across all ligand-receptor pairs for a given combination of clusters from (FIG. 34A) (see STAR Methods for details). (FIGS. 34C-34E) Changes in the standardized interaction scores for top ligand-receptor pairs between ancestors of stromal cells and ancestors of iPSCs (FIG. 34C), neural-like cells (FIG. 34D), and trophoblast cells (FIG. 34E).

FIGS. 35A-35B—Related to FIGS. 29A-29D: Comparison with alternate methods. (FIG. 35A) Monocle2 computes a graph upon which each cell is embedded. The graph, which consists of 5 segments, is visualized in the upper-left pane. The 5 segments are visualized on our FLE in the 5 remaining panels of (FIG. 35A). Segment 1 (green) consists of day 0 cells together with day 18 Stromal cells. Segments 2 and 3 consist of cells from day 2-8 that supposedly arise from Segment 1 cells. Segment 3 gives rise to Segments 4 (purple) and 5 (red). Segment 4 contains the cells we identify as on the MET region and Segment 5 contains the iPSCs, Trophoblasts, and Neural populations, which Monocle2 infers come directly from the non-proliferative cells in segment 3. (FIG. 35B) URD computes a graph representing random walks from a collection of tips to a root. This graph, which consists of 7 segments, is visualized in the upper-left pane. The 7 segments are visualized on our FLE in the remaining panels of (FIG. 35B). Segment 1 (magenta) contains the day 0 MEF cells. The first bifurcation occurs on day 0.5, where segment 2 (consisting of day 0.5 cells) splits off from segment 3 (consisting of day 12-18 Stromal cells). Segment 2 splits to give rise to Segment 4 (consisting of day 2 cells) and Segment 5 consisting of day 12-18 Trophoblasts and Epithelial cells. Segment 4 splits on day 3 to give rise to Segment 6 (consisting of a diverse population including day 3 cells and day 14-18 iPSCs) and Segment 7 (consisting of a diverse population including day 3 cells and day 12-18 Neural-like cells).

FIGS. 36A-36F—Related to FIGS. 29A-29D: Obox6+Obox6 graphs. (FIGS. 36A-36C) Identical to FIGS. 29A-29C except here we show results for serum conditions. (FIG. 36D) Percentage of Oct4-EGFP+ cells at day 16 of reprogramming from secondary MEFs by lentiviral overexpression of Oct4, Klf4, Sox2, and Myc (OKSM) combined with either Zfp42, Obox6, or an empty control, in either 2i or serum conditions. Oct4-EGFP+ cells were measured by flow cytometry. Plot includes the percentage of Oct4-EGFP+ cells in three biological replicates (for Zfp42 and Obox6 overexpression, or an empty control) from five independent experiments (Exp). (FIG. 36E, FIG. 36F) Number of Oct4-EGFP+ colonies at day 16 of reprogramming from primary MEFs by lentiviral overexpression of individual Oct4, Klf4, Sox2, and Myc combined with either Zfp42, Obox6, or an empty control in (FIG. 36E) 2i and (FIG. 36F) serum conditions. Plot includes the number of Oct4-EGFP+ cells in three biological replicates (for Zfp42 and Obox6 overexpression, or an empty control) from two independent experiments (Exp).

FIG. 37—Effects of GDF9 on reprogramming efficiency.

FIG. 38 shows adding GDF9 to the medium resulted in more iPSCs.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods and systems intended to reflect Waddington's image of marbles rolling within a development landscape. It captures the notion that cells at any position in the landscape have a distribution of both probable origins and probable fates. It seeks to reconstruct both the landscape and probabilistic trajectories from scRNA-seq data at various points along a time course. Specifically, it uses time-course data to infer how the probability distribution of cells in gene-expression space evolves over time, by using the mathematical approach of Optimal Transport (OT). The utility of this method is demonstrated in the context of reprogramming of fibroblasts to induced pluripotent stem cells (iPSCs). However, the same method may be applied to other cell development and biological context where an understanding of cell orgins, trajectories, and fates is needed. For ease of reference, the methods disclosed herein and in their various embodiments may be referred to collectively as “Waddington-OT.” As demonstrated herein, Waddington-OT readily rediscovers known biological features of reprogramming, including that successfully reprogrammed cells exhibit an early loss of fibroblast identity, maintain high levels of proliferation, and undergo a mesenchymal-to-epithelial transition before adopting an iPSC-like state (12). In addition, by exploiting single-cell resolution and the new model, it also extends these results by (1) identifying alternative cell fates, including senescence, apoptosis, neural identity, and placental identity; (2) quantifying the portion of cells in each state at each time point; (3) inferring the probable origin(s) and fate(s) of each cell and cell class at each time point; (4) identifying early molecular markers associated with eventual fates; and (5) using trajectory information to identify transcription factors (TFs) associated with the activation of different expression programs. In particular, TFs that are putative regulators of neural identity, placental identity, and pluripotency during reprogramming, and we experimentally demonstrate that one such TF, Obox6, enhances reprogramming efficiency are provided. Together, the data provide a high-resolution resource for studying the roadmap of reprogramming, and the methods provide a general approach for studying cellular differentiation in natural or induced settings.

Prior to describing implementation of the methods in detail, the following overview and definitions utilized in execution of the method are defined.

scRNA-seq may be obtained from cells using standard techniques known in the art. A collection of mRNA levels for a single cell is called an expression profile and is often represented mathematically by a vector in gene expression space. This is a vector space that has a dimension corresponding to each gene, with the value of the ith coordinate of an expression profile vector representing the number of copies of mRNA for the ith gene. Note that real cells only occupy an integer lattice in gene expression space (because the number of copies of mRNA is an integer), but it is assumed herein that cells can move continuously through a real-valued G dimensional vector space.

As an individual cell changes the genes it expresses over time, it moves in gene expression space and describes a trajectory. As a population of cells develops and grows, a distribution on gene expression space evolves over time. When a single cell from such a population is measured with single cell RNA sequencing, a noisy estimate of the number of molecules of mRNA for each gene is obtained. The measured expression profile of this single cell is represented as a sample from a probability distribution on gene expression space. This sampling captures both (a) the randomness in the single cell RNA sequencing measurement process (due to sub-sampling reads, technical issues, etc.) and (b) the random selection of a cell from a population. This probability distribution is treated as nonparametric in the sense that it is not specified by any finite list of parameters.

A precise mathematical notion for a developmental process as a generalization of a stochastic process is provided below. A goal of the methods disclosed herein is to infer the ancestors and descendants of subpopulations evolving according to an unknown developmental process. While not bound by a particular theory, this may be possible over short time scales because it is reasonable to assume that cells don't change too much and therefore it can be inferred which cells go where.

In certain example embodiments, the following definitions to define a precise notion of the developmental trajectory of an individual cell and its descendants are used. It is a continuous path in gene expression that bifurcates with every cell division. Formally, consider a cell x(o)∈ [symbol] ^{G}. Let k(t)≥0 specify the number of descendants at time t, where k(0)=1. A single cell developmental trajectory is a continuous function

$$x \text{:} [ 0 , T ) \to \underbrace{{ℝ}^{G} \times {ℝ}^{G} \times … \times {ℝ}^{G}}_{k ( t ) times} .$$

This means that x(t) is a k(t)-tuple of cells, each represented by a vector [symbol] ^{G}:

x(t)=(x₁(t), . . . ,xₖ₍ₜ₎(t)).

Cells x₁(t), . . . , xₖ₍ₜ₎(t) as the descendants of x(o).

[symbol] ^{G} and R^{G} are used interchangeably.

Note that the temporal dynamics of an individual cell cannot be directly measured because scRNA-Seq is a destructive measurement process: scRNA-Seq lyses cells so it is only possible to measure the expression profile of a cell at a single point in time. As a result, it is not possible to directly measure the descendants of that cell, and it is (usually) not possible to directly measure which cells share a common ancestor with ordinary scRNA-Seq. Therefore the full trajectory of a specific cell is unobservable. However, one can learn something about the probable trajectories of individual cells by measuring snapshots from an evolving population.

Published methods typically represent the aggregate trajectory of a population of cells with a graph. While this recapitulates the branching path traveled by the descendants of an individual cell, it may over-simplify the stochastic nature of developmental processes. Individual cells have the potential to travel through different paths, but in reality any given cell travels one and only one such path. The methods disclosed herein help to describe this potential, which might not be a represented by a graph as a union of one dimensional paths.

Instead, a developmental process is defined to be a time-varying distribution on gene expression space. The word distribution is used to refer to an object that assigns mass to regions of [symbol] ^{G}. Note that a distinction is made between distribution and probability distribution, which necessarily has total mass 1. Distributions are formally defined as generalized functions (such as the delta function δ_{X}) that act on test functions. A used herein a “distribution” is the same as a measure. One simple example of a distribution of cells is that a set of cells x₁, . . . , xₙ can be represented by the distribution

$$ℙ = {\sum }_{i = 1}^{n} {δ}_{{x}_{i}} .$$

Similarly, a set of single cell trajectories may be represented x₁(t), . . . , xₙ(t) with a distribution over trajectories. A developmental process [symbol] ₜ is a time-varying distribution on gene expression space. A developmental process generalizes the definition of stochastic process. A developmental process with total mass 1 for all time is a (continuous time) stochastic process, i.e. an ordered set of random variables with a particular dependence structure. Recall that a stochastic process is determined by its temporal dependence structure, i.e. the coupling between random variables at different time points. The coupling of a pair of random variables refers to the structure of their joint distribution. The notion of coupling for developmental processes is the same as for stochastic processes, except with general distributions replacing probability distributions.

A coupling of a pair of distributions P, Q on R^{G} is a distribution π on R^{G}×R^{G} with the property that π has P and Q as its two marginals. A coupling is also called a transport map.

As a distribution on the product space R^{G}×R^{G}, a transport map π assigns a number π(A, B) to any pair of sets A, B⊂R^{G}.

π(A,B)=∫_{x∈A}∫_{y∈B}π(x,y)dxdy.

When π is the coupling of a developmental process, this number π(A, B) represents the mass transported from A to B by the developmental process. This is the amount of mass coming from A and going to B. When a particular destination is note specified, the quantity π(A, ⋅) specifies the full distribution of mass coming from A. This action may be referred to as pushing A through the transport map π. More generally, we can also push a distribution μ forward through the transport map π via integration

μ [symbol] ∫π(x,⋅)dμ(x).

The reverse operation is referred to as pulling a set B back through π. The resulting distribution B) encodes the mass ending up at B. Distributions μ can also be pulled back through π in a similar way:

μ [symbol] ∫π(⋅,y)dμ(y).

This may also be referred as back-propagating the distribution μ (and to pushing μ forward as forward propagation).

Recall that a stochastic process is Markov if the future is independent of the past, given the present. Equivalently, it is fully specified by its couplings between pairs of time points. A general stochastic process can be specified by further higher order couplings. Markov developmental processes, which are defined in the same way:

A Markov developmental process Pₜ is a time-varying distribution on R^{G} that is completely specified by couplings between pairs of time points. It is an interesting question to what extent developmental processes are Markov. On gene expression space, they are likely not Markov because, for example, the history of gene expression can influence chromatin modifications, which may not themselves be reflected in the observed expression profile but could still influence the subsequent evolution of the process. However, it is possible that developmental processes could be considered Markov on some augmented space.

A definition of descendants and ancestors of subgroups of cells evolving according to a Markov developmental process is now provided. The earlier definition of descendants is extended as follows: Consider a set of cells S⊂R^{G}, which live at time t₁ are part of a population of cells evolving according to a Markov developmental process Pₜ. Let π denote the transport map for Pₜ from time t₁ to time t₂. The descendants of S at time t₂ are obtained by pushing S through the transport map it. Note that if a developmental process is not Markov, then the descendants of S are not well defined. The descendants would depend on the cells that gave rise to S, which we refer to as the ancestors of S.

Definition 6 (ancestors in a Markov developmental process). Consider a set of cells S ⊂R^{G}, which live at time t₂ and are part of a population of cells evolving according to a Markov developmental process Pₜ. Let π denote the transport map for Pₜ from time t₂ to time t₁. The ancestors of S at time t₁ are obtained by pushing S through the transport map π.

### Empirical Developmental Processes

In certain aspects, a goal of the embodiments disclosed herein is to track the evolution of a developmental process from a scRNA-Seq time course. Suppose we are given input data consisting of a sequence of sets of single cell expression profiles, collected at T different time slices of development. Mathematically, this time series of expression profiles is a sequence of sets S₁, . . . , S_{T} ⊂R^{G} collected at times t₁, . . . , t_{T} ∈R.

Developmental time series. A developmental time series is a sequence of samples from a developmental process Pₜ on R^{G}. This is a sequence of sets S₁, . . . , S_{N} ⊂R^{G}. Each Sᵢ is a set of expression profiles in R^{G} drawn i.i.d from the probability distribution obtained by normalizing the distribution Pₜᵢ to have total mass 1. From this input data, we form an empirical version of the developmental process. Specifically, at each time point tᵢ we form the empirical probability distribution supported on the data x∈Sᵢ is formed. This is summarized in the following definition:

Empirical developmental process. An empirical developmental process \hat{P}ₜ is a time vary-ing distribution constructed from a developmental time course S₁, . . . , S_{N}:

$${\hat{ℙ}}_{{t}_{i}} = \frac{1}{\vert {S}_{i} \vert} {\sum }_{x ∈ {S}_{i}} {δ}_{x} .$$

he empirical developmental process is undefined for t∈/{t₁, . . . , t_{N}}.

Our goal is to recover information about a true, unknown developmental process Pₜ from the empirical developmental process \hat{P}ₜ. The measurement process of single cell RNA-Seq destroys the coupling, and the observed empirical developmental process does not come with an informative coupling between successive time points. Over short time scales, it is reasonable to assume that cells do not change too much and therefore inferences regarding which cells go where and estimate the coupling.

This may be done with optimal transport: the transport map π that minimizes the total work required for redistributing [symbol] to [symbol] is selected. One motivation for minimizing this objective, is a deep relationship between optimal transport and dynamical systems that provides a direct connection to Waddington's landscape: the optimal transport problem can formulated as a least-action advection of one distribution into another according to an unknown velocity field (see Theorem 1 in Section 6 below). At a high level, differentiation follows a velocity field on gene expression space, and the potential inducing this velocity field is in direct correspondence with Waddington's landscape¹.

Optimal Transport for scRNA-Seq Time Series

A process for how to compute probabilistic flows from a time series of single cell gene expression profiles by using optimal transport (S1) is provided. The embodiments disclosed herein show how to compute an optimal coupling of adjacent time points by solving a convex optimization problem.

Optimal transport defines a metric between probability distributions; it measures the total distance that mass must be transported to transform one distribution into another. For two measures P and Q on R^{G}, a transport plan is a measure on the product space R^{G}×R^{G} that has marginals P and Q. In probability theory, this is also called a coupling. Intuitively, a transport plan it can be interpreted as follows: if one picks a point mass at position x, then π(x, ⋅) gives the distribution over points where x might end up.

If c(x, y) denotes the cost² of transporting a unit mass from x to y, then the expected cost under a transport plan π is given by

∫∫c(x,y)(x,y)dxdy.

The optimal transport plan minimizes the expected cost subject to marginal constraints:

$${minimize}_{π} \int \int c ( x , y ) π ( x , y ) dxdy$$

$$subject to \int π ( x , • ) dx = ℚ$$

$$\int π ( • , y ) dy = ℙ .$$

Note that this is a linear program in the variable it because the objective and constraints are both linear in it. Note that the optimal objective value defines the transport distance between P and Q (it is also called the Earthmover's distance or Wasserstein distance). Unlike most other ways to compare distributions (such as KL-divergence or total variation), optimal transport takes the geometry of the underlying space into account. For example, the KL-Divergence is infinite for any two distributions with disjoint support, but the transport distance between two unit masses depends on their separation.

When the measures P and Q are supported on finite subsets of R^{G}, the transport plan is a matrix whose entries give transport probabilities and the linear program above is finite dimensional. In this context, empirical distributions are formed from the sets of samples S₁, . . . , S_{T}:

$${\hat{ℙ}}_{{t}_{i}} = \frac{1}{\vert {S}_{i} \vert} {\sum }_{x ∈ {S}_{i}}^{ } {δ}_{x} ,$$

were δ_{X} denotes the Dirac delta function centered at x∈R^{G}. These empirical distributions \hat{P}ₜ_{ᵢ} are definitely supported, and so it is possible solve the linear program[1] with P=\hat{P}ₜᵢ and Q= [symbol] .

However, the classical formulation [1] does not allow cells to grow (or die) during transportation (because it was designed to move piles of dirt and conserve mass). When the classical formulation is applied to a time series with two distinct subpopulations proliferating at different rates³, the transport map will artificially transport mass between the subpopulations to account for the relative proliferation. Therefore, we modify the classical formulation of optimal transport in equation [1] is modified to allow cells to grow at different rates.

Is it assumed that a cell's measured expression profile x determines its growth rate g(x). This is reasonable because many genes are involved in cell proliferation (e.g. cell cycle genes). It is further assumed g(x) is a known function (based on knowledge of gene expression) representing the exponential increase in mass per unit time, but also note that the growth rate can be allowed to be miss-specified by leveraging techniques from unbalanced transport (S2). In practice, g(x) is defined in terms of the expression levels of genes involved in cell proliferation.

Derivation of Transport with Growth:

For any cell x∈Sᵢ₋₁, let r(x, y) be the fraction of x that transitions towards y. Then the amount of probability mass from x that ends up at y (after proliferation) is

r(x,y)g(x)^{Δ}^{ₜ},

where Δₜ=tᵢ₊₁−tᵢ. The total amount of mass that comes from x can be written two ways:

$${\sum }_{y ∈ {S}_{i + 1}}^{ } r ( x , y ) {g ( x )}^{{Δ}_{t}} \approx {g ( x )}^{{Δ}_{t}} d {\hat{ℙ}}_{{t}_{i}} ( x ) .$$

This gives us a first constraint. Similarly, there is also the constraint that the total mass observed at y is equal to the sum of masses coming from each x and ending up at y. In symbols,

$$d {\hat{ℙ}}_{{t}_{i + 1}} ( y ) {\sum }_{x ∈ {S}_{i}}^{ } {g ( x )}^{{Δ}_{t}} \approx {\sum }_{x ∈ {S}_{i}}^{ } r ( x , y ) {g ( x )}^{{Δ}_{t}} for each y ∈ {S}_{i + 1} .$$

The factor x∈Sᵢg(x)^{Δt} on the left hand side accounts for the overall proliferation of all the cells from Sᵢ. Note that this factor is required so that the constraints are consistent: when one sums up both sides of the first constraint over x, this must equal the result of summing up both sides of the second constraint over y. Finally, for convenience these constraints are rewritten in terms of the optimization variable

π(x,y)=r(x,y)g(x)^{Δ}^{ₜ}.

Therefore, to compute the transport map between the empirical distributions of expression profiles observed at time tᵢ and tᵢ₊₁, the following linear program is set up:

$${minimize}_{π} = {\sum }_{x ∈ {S}_{i}}^{ } {\sum }_{y ∈ {S}_{i + 1}}^{ } c ( x , y ) π ( x , y )$$

$$subject to {\sum }_{x ∈ {S}_{i}}^{ } π ( x , y ) \approx d {\hat{ℙ}}_{{t}_{i + 1}} ( y ) {\sum }_{x ∈ {S}_{i}}^{ } {g ( x )}^{{Δ}_{t}}$$

$${\sum }_{y ∈ {S}_{i + 1}}^{ } π ( x , y ) \approx d {\hat{ℙ}}_{{t}_{i}} ( x ) {g ( x )}^{{Δ}_{t}}$$

Regularization and Algorithmic Considerations:

Fast algorithms have been recently developed to solve an entropically regularized version of the transport linear program (S3). Entropic regularization means adding the entropy H(π)=E_{π} log π to the objective function, which penalizes deterministic transport plans (a purely deterministic transport plan would have only one nonzero entry in each row). Entropic regularization speeds up the computations because it makes the optimization problem strongly convex, and gradient ascent on the dual can be realized by successive diagonal matrix scalings (S3). These are very fast operations. This scaling algorithm has also been extended to work in the setting of unbalanced transport, where equality constraints are relaxed to bounds on KL-divergence (S2). This allows the growth rate function g(x) to be misspecified to some extent.

Both entropic regularization and unbalanced transport may be used. To compute the transport map between the empirical distributions of expression profiles observed at time tᵢ and tᵢ₊₁, the embodiments disclosed herein solve the following optimization problem:

$${minimize}_{π} {\sum }_{x ∈ {S}_{i}} {\sum }_{y ∈ {S}_{i + 1}} c ( x , y ) π ( x , y ) - ϵ ℋ ( π )$$

$$subject to KL [ {\sum }_{x ∈ {S}_{i}} π ( x , y ) \vert \vert d {\hat{ℙ}}_{{t}_{i + 1}} ( y ) {\sum }_{x ∈ {S}_{i}} {g ( x )}^{{Δ}_{t}} ] \leq \frac{1}{{λ}_{1}}$$

$$KL [ {\sum }_{y ∈ {S}_{i + 1}} π ( x , y ) \vert \vert {\hat{ℙ}}_{{t}_{i}} ( x ) {g ( x )}^{{Δ}_{t}} ] \leq \frac{1}{{λ}_{2}}$$

where ε, λ₁ and λ₂ are regularization parameters. This is a convex optimization problem in the matrix variable π∈R^{N}^{ᵢ}^{×N}^{ᵢ₊₁} where Nᵢ=|Sᵢ| is the number of cells sequenced at time tᵢ. It takes about 5 seconds to solve this unbalanced transport problem using the scaling algorithm of Chizat et al. 2016 (S2) on a standard laptop with Nᵢ≈5000. Note that the densities (on the discrete set Sᵢ) of the empirical distributions specified in equation [2] are simply d\hat{P}ₜ (x)=1. However, in principle one could use nonuniform empirical distributions (e.g. i Nᵢ if one wanted to include information about cell quality).

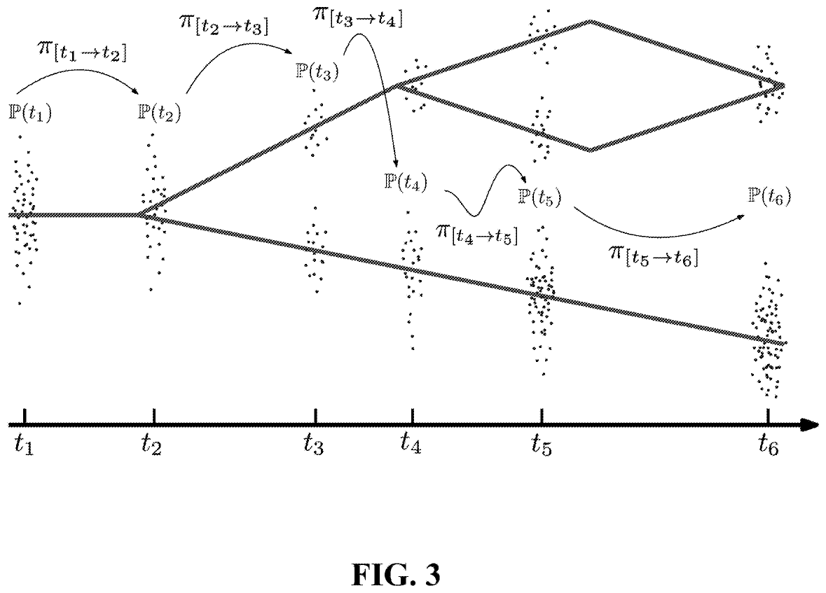

To summarize: given a sequence of expression profiles S₁, . . . , S_{T}, the optimization problem [5] for each successive pair of time points Sᵢ, Sᵢ₊₁ is solved. This gives us a sequence of transport maps as illustrated in FIG. 3.

To make this more precise, consider a single cell y∈Sᵢ. The column π(⋅, y) of the transport map it from tᵢ₋₁ to tᵢ describes the contributions to y of the cells in Sᵢ₋₁. This is the origin of y at the time point tᵢ₋₁. Similarly, the row r(y, ⋅) of the transition map from tᵢ to tᵢ₊₁ describes the probabilities y would transition to cells in Sᵢ₊₁. These are the fates of y, i.e. the descendants of y.

The origin of y further back in time may be computed via matrix multiplication: the contributions to y of cells in Sᵢ₋₂ are given by a column of the matrix

\tilde{π}_{[i-2,i]}=π_{[i-2,i-1]}π_{[i-1,i]}.

This matrix [symbol] represents the inferred transport from time point tᵢ₋₂ to tᵢ, and note it with a tilde to distinguish it from the maps computed directly from adjacent time points. Note that, in principle, the transport between any non-consecutive pairs of time points Sᵢ, Sⱼ, may be directly computed but it is not anticipated that the principle of optimal transport to be as reliable over long time gaps.

Finally, note that expression profiles can be interpolated between pairs of time points by averaging a cell's expression profile at time tᵢ with its fated expression profiles at time tᵢ₊₁.

### Transport Maps Encode Regulatory Information

Transport maps can encode regulatory information, and provided herein are methods on how to set up a regression to fit a regulatory function to our sequence of transport maps. It is assumed that a cell's trajectory is cell-autonomous and, in fact, depends only on its own internal gene expression. We know this is wrong as it ignores paracrine signaling between cells, and we return to discuss models that include cell-cell communication at the end of this section. However, this assumption is powerful because it exposes the time-dependence of the stochastic process Pₜ as arising from pushing an initial measure through a differential equation:

\dot{x}=ƒ(x).

Here f is a vector field that prescribes the flow of a particle x (see FIG. 3 for a cartoon illustration of a distribution flowing according to a vector field). Our biological motivation for estimating such a function f is that it encodes information about the regulatory networks that create the equations of motion in gene-expression space.

We propose to set up a regression to learn a regulatory function f that models the fate of a cell at time tᵢ₊₁ as a function of its expression profile at time tᵢ. For motivation that the transport maps might contain information about the underlying regulatory dynamics, we appeal to a classical theorem establishing a dynamical formulation of optimal transport.

Theorem 1 (Benamou and Brenier, 2001). The optimal objective value of the transport problem [1] is equal to the optimal objective value of the following optimization problem.

$${minimize}_{ρ , v} {\int }_{0}^{1} {\int }_{{ℝ}^{G}} {‖ v ( t , x ) ‖}^{2} ρ ( t , x ) dtdx$$

$$subject to ρ ( 0 , \cdot ) = ℙ , ρ ( 1 , \cdot ) = ℚ . \\ ∇ \cdot ( ρ v ) = \frac{\partial ρ}{\partial t} .$$

In this theorem, v is a vector-valued velocity field that advects4 the distribution ρ from P to Q, and the objective value to be minimized is the kinetic energy of the flow (mass×squared velocity). Intuitively, the theorem shows that a transport map it can be seen as a point-to-point summary of a least-action continuous time flow, according to an unknown velocity field. While the optimization problem [8] can be reformulated as a convex optimization problem, and modified to allow for variable growth rates, it is inherently infinite dimensional and therefore difficult to solve numerically.

We therefore propose a tractable approach to learn a static regulatory function f from our sequence of transport maps. Our approach involves sampling pairs of points using the couplings from optimal transport, and solving a regression to learn a regulatory function that predicts the fate of a cell at time tᵢ₊₁ as a function of its expression profile at time tᵢ:

Regulatory Network Regression:

For each pair of time points tᵢ,tᵢ₊₁, we consider the pair of random variables Xₜ,Xₜ jointly distributed according to r[t,t], (which we obtained from the i i+1 i i+1 transport map π[tᵢ,tᵢ₊₁] by removing the effect of proliferation as in equation [3]). We set up the following optimization problem over regulatory functions f:

$${min}_{f ∈ ℱ} {𝔼}_{r} {‖ \frac{{X}_{{t}_{i}} - {X}_{{t}_{i + 1}}}{{Δ}_{t}} - f ( {X}_{{t}_{i}} ) ‖}^{2} .$$

Here F specifies a parametric function class to optimize over.

Cell Non-Autonomous Processes:

We conclude our treatment of gene regulatory networks by discussing an approach to cell-cell communication. Note that the gradient flow [8] only makes sense for cell autonomous processes. Otherwise, the rate of change in expression x is not just a function of a cell's own expression vector x(t), but also of other expression vectors from other cells. We can accommodate cell non-autonomous processes by allowing f to also depend on the full distribution Pₜ

$$\frac{d x}{d t} = f ( x , {ℙ}_{t} ) .$$

### 4. Extensions to Continuous Time.

In this section we discuss how our method could be improved by going beyond pairs of time points to track the continuous evolution of Pₜ. We begin by pointing out a peculiar behavior of our method: whenever we have a time point with few sampled cells, our method is forced through an information bottleneck. As an extreme example—suppose we had a time point with only one cell. Everything would transition through that single cell, which is absurd! In this extreme case, we would be better off ignoring the time point. We therefore propose a smoothed approach that shares information between time slices and gracefully improves as data is added.

Our continuous-time formulation is based on locally-weighted averaging, an elementary interpolation technique. Recall that given noisy function evaluations yᵢ≈f(xᵢ), one can interpolate f by averaging the yᵢ for all xᵢ close to a point of interest x:

$$f ( x ) \approx {\sum }_{i} {α}_{i} f ( {x}_{i} ) ,$$

where αᵢ are weights that give more influence to nearby points

In our setup, we seek to interpolate a distribution-valued function Pₜ from the collections of i.i.d. samples S₁, . . . , S_{T}. We can interpolate a distribution-valued function by computing the barycenter (or centroid) of nearby time points with respect to the optimal transport metric. The transport barycenter of

$${minimize}_{ℚ} {\sum }_{i = 1}^{T} {α}_{i} {W}^{2} ( {ℙ}_{i} , ℚ )$$

where W (P, Q) denotes the transport distance (or Wasserstein distance) between P and Q. The transport distance is defined by the optimal value of the transport problem [1]. The weights αᵢcan be chosen to interpolate about time point t by setting, for example,

$${minimize}_{ℚ} {\sum }_{i = 1}^{T} {α}_{i} {G}^{2} ( {\hat{ℙ}}_{{t}_{i}} , ℚ )$$

where G(P, Q) denotes our modified transport distance from equation [5]. To solve this optimization problem, we can fix the support of Q to the samples observed at all time points ∪Tᵢ₌₁Sᵢ. Then we can apply the scaling algorithm for unbalanced bary centers due to Chizatetal. (S2).

However, fixing the support of the barycenter ahead of time may not be completely satisfactory, and this motivates further research in the computation of transport barycenters: can we design an algorithm to solve for the barycenter Q without fixing the support in advance? Is there a dynamic formulation for barycenters analogous to the Brenier Benamou formula of Theorem 1, and can we leverage it to better learn gene regulatory networks?

Finally, we conclude this section with the observation that this continuous-time approach could pro-vide a principled approach to sequential experimental design. We can identify optimal time points for further data collection by examining the loss function (fit of barycenter) across time, and adding data where the fit is poor. Moreover, we could also use this continuous time approach to test the principle of optimal transport by withholding some time points and testing the quality of the interpolation against the held-out truth.

### Example System Architectures

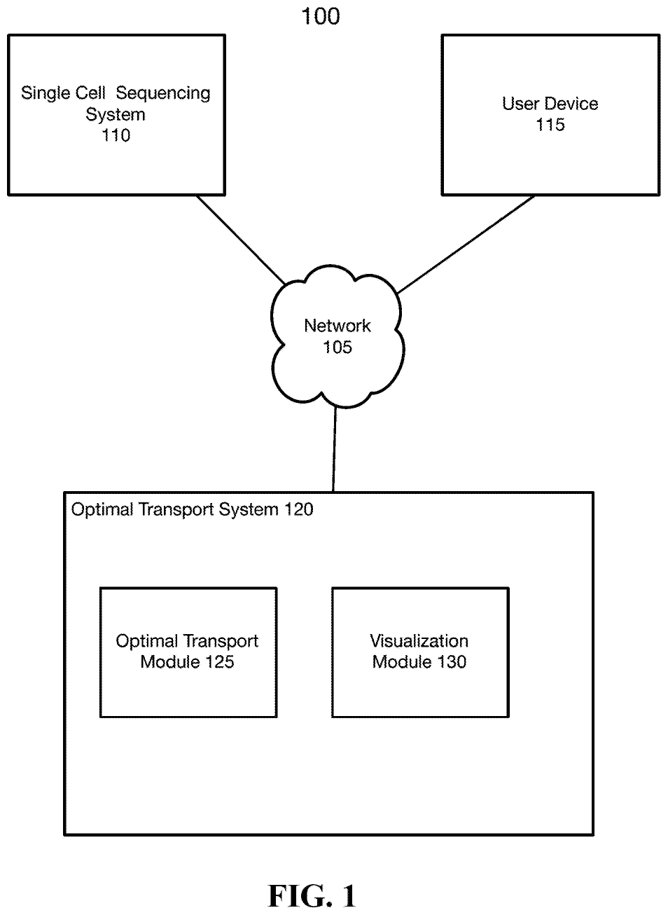

FIG. 1 is a block diagram depicting a system for mapping developmental trajectories of cells using single cell sequencing data, in accordance with certain example embodiments. As depicted in FIG. 1, the system 100 includes network devices 110, 115, and 120, that are configured to communicate with one another via one or more networks 105. In some embodiments, a user associated with the user device 115, may have to install an application and/or make a feature selection to obtain the benefits of the techniques described herein.

Each network 105 includes a wired or wireless telecommunication means by which network devices (including devices 110, 135 and 140) can exchange data. For example, each network 105 can include a local area network (“LAN”), a wide area network (“WAN”), an intranet, an Internet, a mobile telephone network, or any combination thereof. Throughout the discussion of example embodiments, it should be understood that the terms “data” and “information” are used interchangeably herein to refer to text, images, audio, video, or any other form of information that can exist in a computer-based environment.

Each network device 110, 135 and 140 includes a device having a communication module capable of transmitting and receiving data over the network 105. For example, each network device 110, 135 and 140 can include a server, desktop computer, laptop computer, tablet computer, a television with one or more processors embedded therein and/or coupled thereto, smart phone, handheld computer, personal digital assistant (“PDA”), or any other wired or wireless, processor-driven device. In the example embodiment depicted in FIG. 1, the network devices (including systems 110, 115 and 120) are operated by end-users or consumers, merchant operators (not depicted), and feedback system operators (not depicted), respectively.

A user can use the application 112, such as a web browser application or a stand-alone application, to view, download, upload, or otherwise access documents or web pages via a distributed network 105. The network 105 includes a wired or wireless telecommunication system or device by which network devices (including devices 110, 115 and 120) can exchange data. For example, the network 105 can include a local area network (“LAN”), a wide area network (“WAN”), an intranet, an Internet, storage area network (SAN), personal area network (PAN), a metropolitan area network (MAN), a wireless local area network (WLAN), a virtual private network (VPN), a cellular or other mobile communication network, Bluetooth, NFC, or any combination thereof or any other appropriate architecture or system that facilitates the communication of signals, data, and/or messages. Throughout the discussion of example embodiments, it should be understood that the terms “data” and “information” are used interchangeably herein to refer to text, images, audio, video, or any other form of information that can exist in a computer based environment.

The communication application 112 can interact with web servers or other computing devices connected to the network 105, including the single cell sequencing system 110 and optimal transport system 120.

It will be appreciated that the network connections shown are example and other means of establishing a communications link between the computers and devices can be used. Moreover, those having ordinary skill in the art having the benefit of the present disclosure will appreciate that the single cell sequencing system 110, user device 115, and optimal transport system 120 illustrated in FIG. 1 can have any of several other suitable computer system configurations. For example a user device 115 embodied as a mobile phone or handheld computer may not include all the components described above

### Example Processes

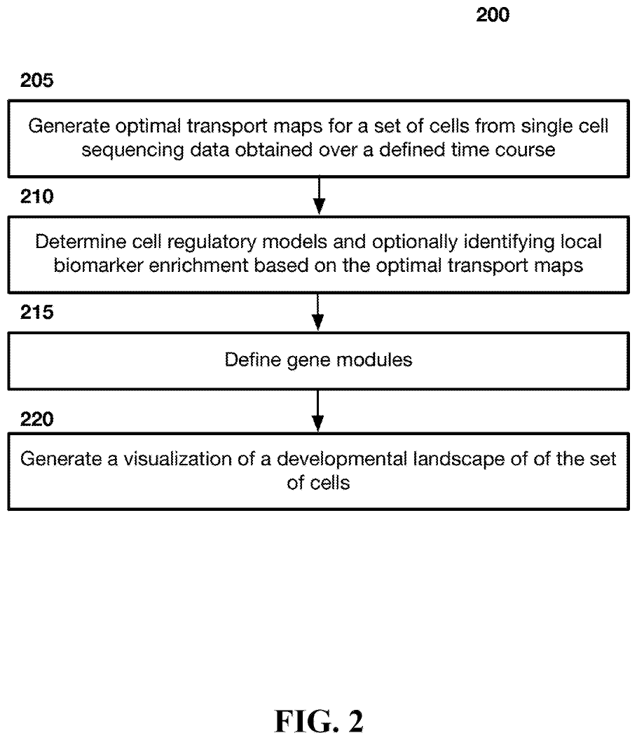

The example methods illustrated in FIG. 2 are described hereinafter with respect to the components of the example operating environment 100. The example methods of FIG. 2 may also be performed with other systems and in other environments

FIG. 2 is a block flow diagram depicting a method 200 to determine developmental trajectories of cells, in accordance with certain example embodiments.

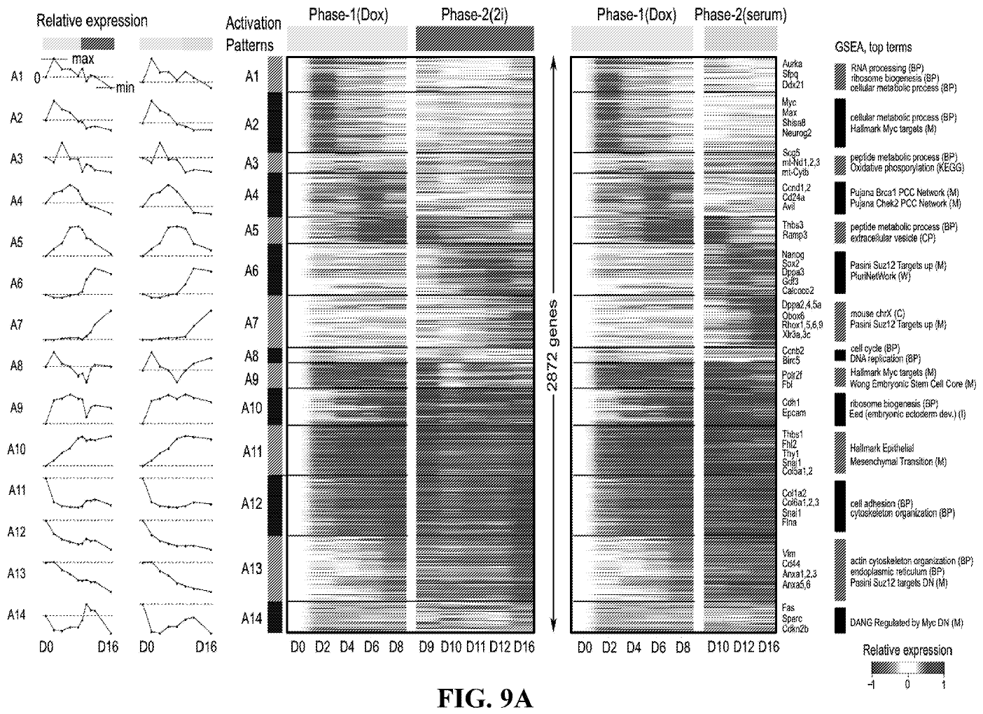

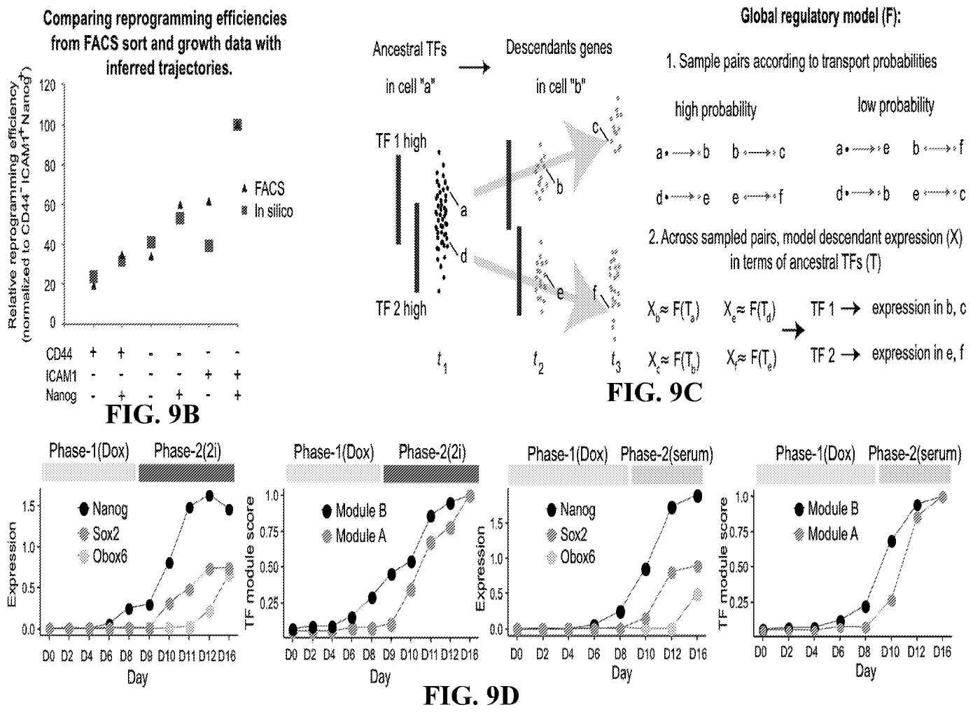

Method 200 begins at block 205, where the optimal transport module 125 performs optimal transport analysis on single cell RNA-seq data (scRNA-seq) from a time course, by calculating optimal transport maps and using them to find ancestors, descendants and trajectories for any set of cells. Given a subpopulation of cells, the sequence of ancestors coming before it and descendants coming after it are referred to as its developmental trajectory. Further example of how development trajectories may be computed in block 205 is described in Example 1 below. Briefly, transport maps are calculated, as described above, between consecutive time points, with cells allowed to grow according to a gene-expression signature of cell proliferation. From these transport maps, the forward and backword transport possibilities can be calculated between any two classes of cells at any time points. For example, a successfully reprogrammed cell at day 16 and use back-propagation to infer the distribution over their precursors at day 12. This can then be further propagated back to day 11, and so one to obtain the ancestor distributions at all previous time points. From this trend in gene expression over time may be plotted. See FIGS. 9A-9D.

In certain example embodiments, an expression matrix may be computed by the optimal transport module 125 from the scRNA-Seq data. Sequence reads may be aligned to obtain a matrix U of UMI counts, with a row for each gene and column for each cell. To reduce variation due to fluctuations in the total number of transcripts per cell, we divide the UMI vector for each cell by the total number of transcripts in that cell. Thus we define the expression matrix E in terms of the UMI matrix U via:

$${E}_{ij} = \frac{{U}_{ij}}{{Σ}_{i = 1}^{G} {U}_{ij}} \times {10}^{4} .$$

Two variance-stabilizing transforms of the expression matrix E may be used for further analysis. In particular
1. [symbol] to be the log-normalized expression matrix. The entries of [symbol] are obtained via

\tilde{E}ᵢⱼ=log(Eᵢⱼ+1).
2. Ē to be the truncated expression matrix. The entries of Ē are obtained by capping the entries of E at the 99.5% quantile.

At block 210, the optimal transport module 125 determines cell regulatory models based on the optimal transport maps. In certain example embodiments, the optimal transport module 125 determines cell regulatory models based at least in part on the optimal transport maps. In certain example embodiments, the optimal transport module 125 may further identify local biomarker enrichment based at least in part on the optimal transport maps. An example implementation is described in further detail in Example 1 below. Transcription factors (TFs) that appear to play important roles along trajectories to key destinations are identified by two approaches. The first approach involves constructing a global regulatory model. Pairs of cells at consecutive time points are sampled according to their transport probabilities; expression levels of Tfs in the cell at time t are used to predict expression levels of all non-TFs in the paired cell at time t+1, under the assumption that the regulatory rules are constant across cells and time points. TFs may be excluded from the predicted set to avoid cases of spurious self-regulation). The second approach involves enrichment analysis. TFs are identified based on enrichment in cells at an earlier time point with a high probability (e.g. >80%) of transitioning to a given state vs. those with a low probability (e.g. <20%).

At block 215, the optimal transport module 125 may further define gene modules. In certain example embodiments, this step is optional. Cells may be clustered based on their gene-expression profiles, after performing two rounds of dimensionality reduction to increase statistical power in subsequent analyses. For the reprogramming data disclosed herein, the analysis partitioned 16,339 detected genes into 44 gene modules, which were then analyzed for enrichment of gene sets (signatures) related to specific pathways, cells types, and conditions. (FIG. 13, Table 1). Based on the expression profiles in each cell, signature scores were calculated (defined by curated gene sets) for relevant features including MEF identity, pluripotency, proliferation, apoptosis, senescence, X-reactivation, neural identity, placental identity and genomic copy-number variation.

TABLE 1

| | Gene | | | |
|---|---|---|---|---|
| Clusters | Modules | ID (Term) | q-Value | Database |
| 1 | GM4 | GO:0036211 (protein modification process) | 7.0 10-3 | BP |
| | GM10 | GO:001604 (cellular component organization) | | BP |
| | | GO:0036211 (protein modification process) | | BP |
| | | GO:0006325 (chromain organization) | | BP |
| | | GO:0016570 (histone modification) | | BP |
| 2 | GM5 | GO:0007049 (cell cycle) | 9.6 10-123 | BP |
| | | GO:0000278 (mitotic cell cycle) | 6.7 10-110 | BP |
| | | GO:0006260 (DNA replication) | 6.7 10-55 | BP |
| 3 | GM33 | IPR001400 (Somatotropin) | 9.0 10-06 | I |
| | | GO:0005179 (hormone activity) | 3.3 10-09 | MF |
| | | R-MMU-1170546 (Prolactin receptor signaling) | 7.0 10-15 | R |
| | | R-MMU-982772 (Growth hormone receptor signaling) | 1.1 10-13 | R |
| | GM40 | GO:0045664 (regulation of neuron differentiation) | | BP |
| 4 | GM8 | GO:0030855 (epithelial cell differentiation) | 2.6 10-11 | BP |
| | | GO:0060429 (epithelium development) | 1.5 10-07 | BP |
| | | mmu04530 (Tight junction) | 2.7 10-08 | K |
| | GM14 | GO:0001890 (placenta development) | 2.5 10-5 | BP |
| | GM42 | GO:0016126 (sterol biosynthetic process) | 4.8 10-38 | BP |
| | | Hallmark cholesterol homeostasis | 8.0 10-29 | M |
| 5 | GM2 | GO:0009653 (anatomical structure morphogenesis) | 5.8 10-29 | BP |
| | | GO:0050793 (regulation of developmental process) | 1.6 10-25 | BO |
| | | GO:0031012 (extracellular matrix) | 1.6 10-17 | CC |
| | GM6 | Lee Bmp2 Targets up | 2.3 10-16 | M |
| | GM7 | GO:0034976 (response to endoplasmic reticulum stress) | 3.8 10-16 | BP |
| | GM9 | GO:0072331 (signal transduction by p53 class mediator) | 6.5 10-06 | BP |
| | | mmu04115 (p53 signaling pathway) | 2.9 10-10 | K |
| | | HALLMARK_P53_PATHWAY | 2.1 10-26 | M |
| | GM23 | GO:0043568 (positive regulation of insulin-like growth | 1.0 10-4 | BP |
| | | factor receptor signaling pathway) | | |
| | | GO:0005520 (insulin-like growth factor binding) | 3.1 10-5 | MF |
| | GM27 | GO:0031012 (extracellular matrix) | 2.9 10-3 | CC |
| | GM32 | GO:0006749 (glutathione metabolic process) | 1.5 10-3 | BP |
| | | MOUSEPWY-4061 (glutathione-mediated detoxification) | 1.7 10-2 | BI |
| | GM34 | GO:0035456 (response to interferon-beta) | 2.5 10-13 | BP |
| | | GO:0006952 (defense response) | 8.0 10-11 | BP |
| | GM35 | GO:0006952 (defense response) | 6.6 10-08 | BP |
| | | GO:0006958 (complement activation, classical pathway) | 1.7 10-5 | BP |
| | GM37 | GO:0034097 (response to cytokine) | 5.0 10-11 | BP |
| | | mmu04668 (TNF signaling pathway) | 4.8 10-11 | K |
| | GM43 | HallmarkTgf beta signaling | 2.0 10-3 | M |
| | GM44 | GO:0009952 (ranterior/posterior pattern specification) | 2.9 10 15 | BP |
| | | GO:0001501 (skeletal system development) | 1.2 10-12 | BP |
| 6 | GM13 | Pasini Suz12 Targets up | 3.0 10-20 | M |
| | | WP1763 PluriNetWork | 3.6 10-06 | W |
| | GM18 | Mikkelsen Pluripotent State up | 2.2 10-3 | M |
| | GM25 | mouse chrX\|X | 1.1 10-3 | C |
| 7 | GM22 | GO:0007399 (nervous system development) | 4.64 10-5 | BP |
| | | GO:0097458 (neuron part) | 2.4 10-5 | CC |

In certain example embodiments, dimensionality reduction may be used to increase robustness. As a first step towards dimensionality reduction, genes that do not show significant variation are removed. The resulting variable-gene expression matrix may be denoted Eᵥₐᵣ.

A second round of dimensionality reduction may comprise non-linear mapping such as Laplacian embedding, or diffusion component embedding. While principal component analysis (PCA) is a traditional approach to reduce dimensionality, it is only typically appropriate for preserving linear structures. To accommodate nonlinear shapes in high-dimensional gene expression space, diffusion components which are a generalization of principal components were used.

The diffusion components defined in terms of a similarity function k: RG×RG→[0, ∞). For a pair (x, y) of G-dimensional gene-expression profiles, the similarity function—or kernel function—k(x, y) measures the similarity between x and y. We use the Gaussian kernel function

$$k ( x , y ) = {e}^{- \frac{{‖ \tilde{x} - \tilde{y} ‖}^{2}}{2 {σ}^{2}}} .$$

Where x and y are log-transformed expression profiles (i.e. columns of \tilde{E}′,)

The diffusion components are defined as the top eigenvectors of a certain matrix constructed by evaluating the kernel function for all pairs of expression profiles x₁, . . . , X_{N}. Specifically, the kernel matrix K is formed with entries

Kᵢⱼ=k(xᵢ,xⱼ),

and then the Laplacian matrix L is formed by multiplying K on the left and the right by D^{-1/2}, where D is a diagonal matrix with entries

$${D}_{i i} = {\sum }_{j = 1}^{N} k ( {x}_{i} , {x}_{j} ) .$$

The Laplacian matrix L is given by

$$L = {D}^{- \frac{1}{2}} K {D}^{- \frac{1}{2}} .$$

The diffusion components are the eigenvectors v₁, . . . , v_{N} of L, sorted by eigenvalue. We embed the data in d dimensional diffusion component space by selecting the top d diffusion components v1, . . . , vd, and sending data point xi to the vector obtained by selecting the ith entry of v1, . . . , v20. The diffusion component embedding of an expression profile x may be denoted by Φd(x). The top 20 diffusion components were enriched for gene signatures related to biological processes, and therefore were elected to use the top 20 diffusion components to represent data (see below for details).

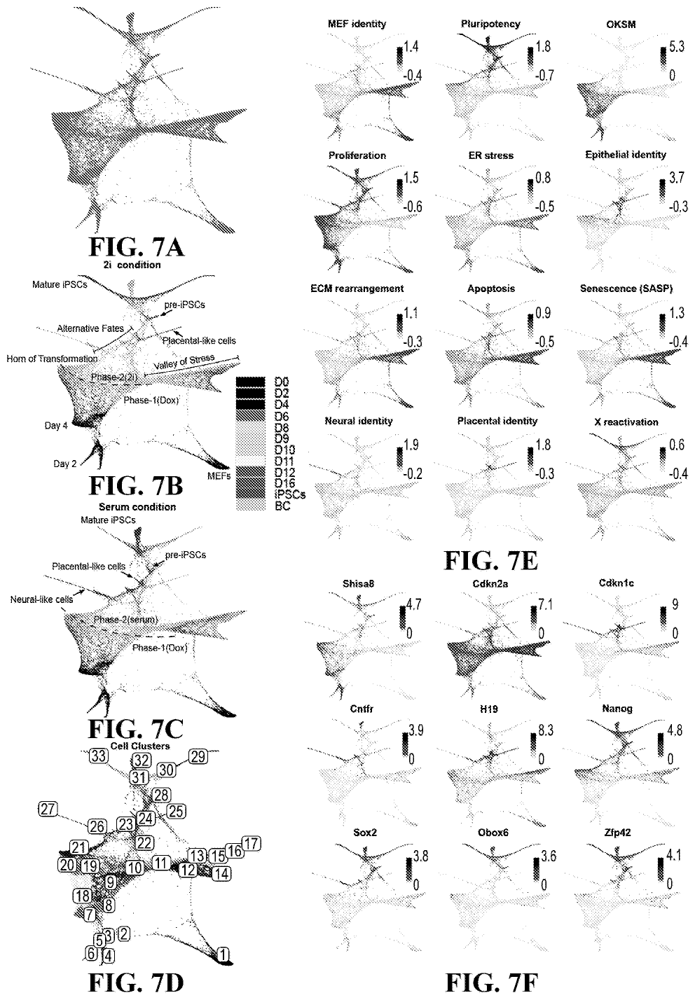

At block 215, the visualization module 130 generates a visualization of a developmental landscape of the set of cells. To visualize the developmental landscape, the dimensionality of the data is reduced with diffusion components (such as those described above), and then the data is embedded in two dimension with force-directed graph visualization. While alternative visualization methods, such as t-distributed Stochastic Neighbor Embedding (t-SNE), are well suited for identifying clusters, they do not preserve global structures by including repulsive forces between dissimilar points. In particular, these repulsive forces seem to do a good job of splaying out the spikes present in the diffusion map embedding. FIGS. 7A-7F.

The invention is further described in the following examples, which do not limit the scope of the invention described in the claims.

### Methods for Inducing Pluripotent Stems Cell

The invention provides for a method of producing an induced pluripotent stem cell comprising introducing Obox6 into a target cell to produce an induced pluripotent stem cell. In one embodiment, a nucleic acid encoding Obox6 is introduced into a target cell. The method may include a step of introducing into the target cell at least one nucleic acid encoding a reprogramming factor selected from the group consisting of: Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sal11, Sal14, Esrrb, Nr5a2, Tbx3, and Glis1, or selected from the group consisting of: Oct4, Klf4, Sox2 and Myc.

In one embodiment, the nucleic acid encoding Obox6 is provided in a recombinant vector, for example, a lentivirus vector. In another embodiment, the nucleic acid encoding the reprogramming factor is provided in a recombinant vector. The nucleic acid may be incorporated into the genome of the cell. The nucleic may not be incorporated into the genome of the cell.

The method may include a step of culturing the cells in reprogramming medium as defined herein. The method may also include a step of culturing the cells in the presence of serum or the absence of serum, for example, after a culturing step in reprogramming medium.

The induced pluripotent stem cell produced according to the methods of the invention can express at least one of a surface marker selected from the group consisting of: Oct4, SOX2, KLf4, c-MYC, LIN28, Nanog, Glis1, TRA-160/TRA-1-81/TRA-2-54, SSEA1, SSEA4, Sal4 and Esrbb1.

The method can be performed with a target cell that is a mammalian cell, including but not limited to a human, murine, porcine or canine cell. The target cell can be a primary or secondary mouse embryonic fibroblast (MEF).The target cell can be any one of the following: fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, pancreatic cells, hepatocytes, macrophages, monocytes, mononuclear cells, and gastric cells, including gastric epithelial cells.

The target cell can be embryonic, or adult somatic cells, differentiated cells, cells with an intact nuclear membrane, non-dividing cells, quiescent cells, terminally differentiated primary cells, and the like.

The invention also provides for a method of producing an induced pluripotent stem cell comprising introducing at least one of Obox6, Spic, Zfp42, Sox2, Mybl2, Msc, Nanog, Hesx1 and Esrrb into a target cell to produce an induced pluripotent stem cell. In one embodiment, a nucleic acid encoding Obox6, Spic, Zfp42, Sox2, Mybl2, Msc, Nanog, Hesx1 or Esrrb is introduced into a target cell.

The invention also provides a method of producing an induced pluripotent stem cell comprising introducing at least one of the transcription factors identified in Table 2, Table 3, Table 4, Table 5 or Table 6 into a target cell to produce an induced pluripotent stem cell. In one embodiment, a nucleic acid encoding a transcription factor identified in Table 2, Table 3, Table 4, Table 5 or Table 6 is introduced into a target cell.

TABLE 2
Genes detected in less than 1% of cells in clusters 1-27

Rhox2a
Myo1f
Xlr3c
Stra8
Smtnl1
Tspo2
Aurkc
Dazl
Rhox1
Crxos
Rbakdn
Smc1b
Tuba3a
Sycp3
Apobec2
Obox6
Patl2
Platr3
Gpx6
1700013H16Rik
Lncenc1
Tcl1
Spic
Hsf2bp
Fkbp6
Arl14epl
Pacsin1
Fam183b
Dpys
Fmr1nb
Gm9732
Dppa4
Fam25c
Dppa2
Lrrc34
Trpm1
Khdc3
Col9a2
Mageb16
Hesx1
Myl7
Ly6g6e
Gm9
Gm13580
Aard
Zfp42
Gm7325

TABLE 3

| | frequency in high/ | frequency | frequency |
|---|---|---|---|
| TF | frequency in low | in high | in low |
| Spic | 15.63 | 38.5% | 2.4% |
| Zfp42 | 17.41 | 33.4% | 1.9% |
| Obox6 | 61.90 | 9.3% | 0.1% |
| Sox2 | 11.68 | 33.5% | 2.9% |
| Mybl2 | 22.55 | 17.2% | 0.7% |
| Msc | 20.37 | 16.9% | 0.8% |
| Nanog | 6.08 | 51.3% | 8.4% |
| Hesx1 | 8.68 | 35.5% | 4.1% |
| Esrrb | 17.00 | 16.4% | 1.0% |

Bold: Intersection between global regulatory network and enrichment analysis

TABLE 4
Late pluripotency markers unique to successful trajectory
Genes detected in less than 1% of cells in clusters 1-27

Rhox2a
Myo1f
Xlr3c
Stra8
Smtnl1
Tspo2
Aurkc
Dazl
Rhox1
Crxos
Rbakdn
Smc1b
Tuba3a
Sycp3
Apobec2
Obox6
Patl2
Platr3
Gpx6
1700013H16Rik
Lncenc1
Tcl1
Spic
Hsf2bp
Fkbp6
Arl14epl
Pacsin1
Fam183b
Dpys
Fmr1nb
Gm9732
Dppa4
Fam25c
Dppa2
Lrrc34
Trpm1
Khdc3
Col9a2
Mageb16
Hesx1
Myl7
Ly6g6e
Gm9
Gm13580
Aard
Zfp42
Gm7325

TABLE 5

| | frequency in high/ | frequency | frequency |
|---|---|---|---|
| TF | frequency in low | in high | in low |
| Spic | 15.63 | 38.5% | 2.4% |
| Zfp42 | 17.41 | 33.4% | 1.9% |
| Obox6 | 61.90 | 9.3% | 0.1% |
| Sox2 | 11.68 | 33.5% | 2.9% |
| Mybl2 | 22.55 | 17.2% | 0.7% |
| Msc | 20.37 | 16.9% | 0.8% |
| Nanog | 6.08 | 51.3% | 8.4% |
| Hesx1 | 8.68 | 35.5% | 4.1% |
| Esrrb | 17.00 | 16.4% | 1.0% |

Bold: Intersection between global regulatory network and enrichment analysis

TABLE 6
Candidate Transcription Factors

| Gene | Description | Reference |
|---|---|---|
| Spic | Spi-C transcription factor | Roderick T H, Chromosomal inversions in |
| | (Spi-1/PU.1 related) | studies of mammalian mutagenesis. |
| | | Genetics. 1979 May; 92(1 Pt 1 |
| | | Suppl): s121-6 |
| Zfp42 | zinc finger protein 42 | Hosler B A, et al., Expression of REX-1, a |
| | | gene containing zinc finger motifs, is |
| | | rapidly reduced by retinoic acid in F9 |
| | | teratocarcinoma cells. Mol Cell Biol. 1989 |
| | | December; 9(12): 5623-9 |
| Obox6 | oocyte specific homeobox 6 | Ko M S, et al., Large-scale cDNA analysis |
| | | reveals phased gene expression patterns |
| | | during preimplantation mouse |
| | | development. Development. 2000 |
| | | April; 127(8): 1737-49 |
| Sox2 | SRY (sex determining region | Lyon M F, et al., Dose-response curves for |
| | Y)-box 2 | radiation-induced gene mutations in mouse |
| | | oocytes and their interpretation. Mutat Res. |
| | | 1979 November; 63(1): 161-73 |
| Mybl2 | myeloblastosis oncogene-like | Lam E W, et al., Characterization and cell |
| | 2 | cycle-regulated expression of mouse B- |
| | | myb. Oncogene. 1992 September; 7(9): 1885-90 |
| Msc | musculin | Robb L, et al., musculin: a murine basic |
| | | helix-loop-helix transcription factor gene |
| | | expressed in embryonic skeletal muscle. |
| | | Mech Dev. 1998 August; 76(1-2): 197-201 |
| Nanog | Nanog homeobox | Kawai J, et al., Functional annotation of a |
| | | full-length mouse cDNA collection. |
| | | Nature. 2001 February 8; 409(6821): 685-90 |
| Hesx1 | homeobox gene expressed in | Thomas P Q, et al., HES-1, a novel |
| | ES cells | homeobox gene expressed by murine |
| | | embryonic stem cells, identifies a new |
| | | class of homeobox genes. Nucleic Acids |
| | | Res. 1992 November 11; 20(21): 5840 |
| Esrrb | estrogen related receptor, | Pettersson K, et al., Expression of a novel |
| | beta | member of estrogen response element- |
| | | binding nuclear receptors is restricted to |
| | | the early stages of chorion formation |
| | | during mouse embryogenesis. Mech Dev. |
| | | 1996 February; 54(2): 211-23 |
| Rhox2a | reproductive homeobox 2A | Kawai J, et al., Functional annotation of a |
| | | full-length mouse cDNA collection. |
| | | Nature. 2001 February 8; 409(6821): 685-90 |
| Myo1f | myosin IF | Hasson T, et al., Mapping of |
| | | unconventional myosins in mouse and |
| | | human. Genomics. 1996 September 15; 36(3): 431-9 |
| Xlr3c | X-linked lymphocyte- | Bergsagel P L, et al., Sequence and |
| | regulated 3C | expression of murine cDNAs encoding |
| | | Xlr3a and Xlr3b, defining a new X-linked |
| | | lymphocyte-regulated Xlr gene subfamily. |
| | | Gene. 1994 December 15; 150(2): 345-50 |
| Stra8 | stimulated by retinoic acid | Bouillet P, et al., Efficient cloning of |
| | gene 8 | cDNAs of retinoic acid-responsive genes |
| | | in P19 embryonal carcinoma cells and |
| | | characterization of a novel mouse gene, |
| | | Stra1 (mouse LERK-2/Eplg2). Dev Biol. |
| | | 1995 August; 170(2): 420-33 |
| Smtnl1 | smoothelin-like 1 | Kawai J, et al., Functional annotation of a |
| | | full-length mouse cDNA collection. |
| | | Nature. 2001 February 8; 409(6821): 685-90 |
| Tspo2 | translocator protein 2 | Kawai J, et al., Functional annotation of a |
| | | full-length mouse cDNA collection. |
| | | Nature. 2001 February 8; 409(6821): 685-90 |
| Aurkc | aurora kinase C | Tseng T C, et al., Protein kinase profile of |
| | | sperm and eggs: cloning and |
| | | characterization of two novel testis- |
| | | specific protein kinases (AIE1, AIE2) |
| | | related to yeast and fly chromosome |
| | | segregation regulators. DNA Cell Biol. |
| | | 1998 October; 17(10): 823-33 |
| Dazl | deleted in azoospermia-like | Kasahara M, et al., Genetic mapping of a |
| | | male germ cell-expressed gene Tpx-2 to |
| | | mouse chromosome 17. Immunogenetics. |
| | | 1991; 34(2): 132-5 |
| Rhox1 | reproductive homeobox 1 | Maclean J A 2nd, et al., Rhox: a new |
| | | homeobox gene cluster. Cell. 2005 February |
| | | 11; 120(3): 369-82 |
| Crxos | cone-rod homeobox, opposite | Ko M S, et al., Large-scale cDNA analysis |
| | strand | reveals phased gene expression patterns |
| | | during preimplantation mouse |
| | | development. Development. 2000 |
| | | April; 127(8): 1737-49 |
| Rbakdn | RB-associated KRAB zinc | MGD Nomenclature Committee, |
| | finger downstream neighbor | February 14, 1995; |
| | (non-protein coding) | |
| Smc1b | structural maintenance of | Biswas U, et al., Distinct Roles of Meiosis- |
| | chromosomes 1B | Specific Cohesin Complexes in |
| | | Mammalian Spermatogenesis. PLoS |
| | | Genet. 2016 October; 12(10): e1006389 |
| Tuba3a | tubulin, alpha 3A | Villasante A, et al., Six mouse alpha- |
| | | tubulin mRNAs encode five distinct |
| | | isotypes: testis-specific expression of two |
| | | sister genes. Mol Cell Biol. 1986 |
| | | July; 6(7): 2409-19 |
| Sycp3 | synaptonemal complex protein | Roderick T H, Chromosomal inversions in |
| | 3 | studies of mammalian mutagenesis. |
| | | Genetics. 1979 May; 92(1 Pt 1 |
| | | Suppl): s121-6 |
| Apobec2 | apolipoprotein B mRNA | Hirano K, et al., Targeted disruption of the |
| | editing enzyme, catalytic | mouse apobec-1 gene abolishes |
| | polypeptide 2 | apolipoprotein B mRNA editing and |
| | | eliminates apolipoprotein B48. J Biol |
| | | Chem. 1996 April 26; 271(17): 9887-90 |
| Obox6 | oocyte specific homeobox 6 | Ko M S, et al., Large-scale cDNA analysis |
| | | reveals phased gene expression patterns |
| | | during preimplantation mouse |
| | | development. Development. 2000 |
| | | April; 127(8): 1737-49 |
| Patl2 | protein associated with | Marnef A, et al., Distinct functions of |
| | topoisomerase II homolog 2 | maternal and somatic Pat1 protein |
| | | paralogs. RNA. 2010 November; 16(11): 2094- |
| | | 107 |
| Platr3 | pluripotency associated | Leo D, et al., Transgenic mouse models for |
| | transcript 3 | ADHD. Cell Tissue Res. 2013 May 17 |
| Gpx6 | glutathione peroxidase 6 | Roderick T H, Producing and detecting |
| | | paracentric chromosomal inversions in |
| | | mice. Mutat Res. 1971 January; 11(1): 59-69 |
| 1700013H16Rik | RIKEN cDNA 1700013H16 | Kawai J, et al., Functional annotation of a |
| | gene | full-length mouse cDNA collection. |
| | | Nature. 2001 February 8; 409(6821): 685-90 |
| Lncenc1 | long non-coding RNA, | Lai K M, et al., Diverse Phenotypes and |
| | embryonic stem cells | Specific Transcription Patterns in Twenty |
| | expressed 1 | Mouse Lines with Ablated LincRNAs. |
| | | PLoS One. 2015; 10(4): e0125522 |
| Tcl1 | T cell lymphoma breakpoint 1 | Narducci M G, et al., The murine Tcl1 |
| | | oncogene: embryonic and lymphoid cell |
| | | expression. Oncogene. 1997 August |
| | | 18; 15(8): 919-26 |
| Spic | Spi-C transcription factor | Roderick T H, Chromosomal inversions in |
| | (Spi-1/PU.1 related) | studies of mammalian mutagenesis. |
| | | Genetics. 1979 May; 92(1 Pt 1 |
| | | Suppl): s121-6 |
| Hsf2bp | heat shock transcription | Kawai J, et al., Functional annotation of a |
| | factor | full-length mouse cDNA collection. |
| | 2 binding protein | Nature. 2001 February 8; 409(6821): 685-90 |
| Fkbp6 | FK506 binding protein 6 | Coss M C, et al., Molecular cloning, DNA |
| | | sequence analysis, and biochemical |
| | | characterization of a novel 65-kDa FK506- |
| | | binding protein (FKBP65). J Biol Chem. |
| | | 1995 December 8; 270(49): 29336-41 |
| Arl14epl | ADP-ribosylation factor-like | Zambrowicz B P, et al., Wnk1 kinase |
| | 14 effector protein-like | deficiency lowers blood pressure in mice: a |
| | | gene-trap screen to identify potential |
| | | targets for therapeutic intervention. Proc |
| | | Natl Acad Sci USA. 2003 November |
| | | 25; 100(24): 14109-14 |
| Pacsin1 | protein kinase C and casein | Plomann M, et al., PACSIN, a brain |
| | kinase substrate in neurons 1 | protein that is upregulated upon |
| | | differentiation into neuronal cells. Eur J |
| | | Biochem. 1998 August 15; 256(1): 201-11 |
| Fam183b | family with sequence | Roderick T H, Chromosomal inversions in |
| | similarity 183, member B | studies of mammalian mutagenesis. |
| | | Genetics. 1979 May; 92(1 Pt 1 |
| | | Suppl): s121-6 |
| Dpys | dihydropyrimidinase | Skarnes W C, et al., A conditional |
| | | knockout resource for the genome-wide |
| | | study of mouse gene function. Nature. |
| | | 2011 June 16; 474(7351): 337-42 |
| Fmr1nb | fragile X mental retardation 1 | Skarnes W C, et al., A conditional |
| | neighbor | knockout resource for the genome-wide |
| | | study of mouse gene function. Nature. |
| | | 2011 June 16; 474(7351): 337-42 |
| Gm9732 | predicted gene 9732 | Roderick T H, Using inversions to detect |
| | | and study recessive lethals and |
| | | detrimentals in mice, in Utilization of |
| | | Mammalian Specific Locus Studies in |
| | | Hazard Evaluation and Estimation of |
| | | Genetic Risk. 1983: 135-67. |
| Dppa4 | developmental pluripotency | Ko M S, et al., Large-scale cDNA analysis |
| | associated 4 | reveals phased gene expression patterns |
| | | during preimplantation mouse |
| | | development. Development. 2000 |
| | | April; 127(8): 1737-49 |
| Fam25c | family with sequence | Kawai J, et al., Functional annotation of a |
| | similarity 25, member C | full-length mouse cDNA collection. |
| | | Nature. 2001 February 8; 409(6821): 685-90 |
| Dppa2 | developmental pluripotency | Ko M S, et al., Large-scale cDNA analysis |
| | associated 2 | reveals phased gene expression patterns |
| | | during preimplantation mouse |
| | | development. Development. 2000 |
| | | April; 127(8):1737-49 |
| Lrrc34 | leucine rich repeat containing | Kawai J, et al., Functional annotation of a |
| | 34 | full-length mouse cDNA collection. |
| | | Nature. 2001 February 8; 409(6821): 685-90 |
| Trpm1 | transient receptor potential | Dickinson M E, et al., High-throughput |
| | cation channel, subfamily M, | discovery of novel developmental |
| | member 1 | phenotypes. Nature. 2016 September |
| | | 14; 537(7621): 508-514 |
| Khdc3 | KH domain containing 3, | Kawai J, et al., Functional annotation of a |
| | subcortical maternal complex | full-length mouse cDNA collection. |
| | member | Nature. 2001 February 8; 409(6821): 685-90 |
| Col9a2 | collagen, type IX, alpha 2 | Dickinson M E, et al., High-throughput |
| | | discovery of novel developmental |
| | | phenotypes. Nature. 2016 September |
| | | 14; 537(7621): 508-514 |
| Mageb16 | melanoma antigen family B, | Kawai J, et al., Functional annotation of a |
| | 16 | full-length mouse cDNA collection. |
| | | Nature. 2001 February 8; 409(6821): 685-90 |
| Hesx1 | homeobox gene expressed in | Thomas P Q, et al., HES-1, a novel |
| | ES cells | homeobox gene expressed by murine |
| | | embryonic stem cells, identifies a new |
| | | class of homeobox genes. Nucleic Acids |
| | | Res. 1992 November 11; 20(21): 5840 |
| Myl7 | myosin, light polypeptide 7, | Lowey S, et al., Light chains from fast and |
| | regulatory | slow muscle myosins. Nature. 1971 November |
| | | 12; 234(5324): 81-5 |
| Ly6g6e | lymphocyte antigen 6 | Kawai J, et al., Functional annotation of a |
| | complex, locus G6E | full-length mouse cDNA collection. |
| | | Nature. 2001 February 8; 409(6821): 685-90 |
| Gm9 | predicted gene 9 | The FANTOM Consortium and RIKEN |
| | | Genome Exploration Research Group and |
| | | Genome Science Group (Genome Network |
| | | Project Core Group), The Transcriptional |
| | | Landscape of the Mammalian Genome. |
| | | Science. 2005; 309(5740): 1559-1563 |
| Gm13580 | predicted gene 13580 alanine | Zambrowicz B P, et al., Wnk1 kinase |
| | and arginine rich | deficiency lowers blood pressure in mice: a |
| | | gene-trap screen to identify potential |
| | | targets for therapeutic intervention. Proc |
| | | Natl Acad Sci USA. 2003 November |
| | | 25; 100(24): 14109-14 |
| Aard | domain containing protein | Roderick T H, et al., Nineteen paracentric |
| | | chromosomal inversions in mice. Genetics. |
| | | 1974 January; 76(1): 109-17 |
| Zfp42 | zinc finger protein 42 | Hosier B A, et al., Expression of REX-1, a |
| | | gene containing zinc finger motifs, is |
| | | rapidly reduced by retinoic acid in F9 |
| | | teratocarcinoma cells. Mol Cell Biol. 1989 |
| | | December; 9(12): 5623-9 |
| Gm7325 | myomixer, myoblast fusion | Hansen J, et al., A large-scale, gene-driven |
| | factor | mutagenesis approach for the functional |
| | | analysis of the mouse genome. Proc Natl |
| | | Acad Sci USA. 2003 August |
| | | 19; 100(17): 9918-22 |

The invention also provides a method of increasing the efficiency of production of an induced pluripotent stem cell comprising introducing Obox6 into a target cell to produce an induced pluripotent stem cell.

The invention also provides a method of increasing the efficiency of production of an induced pluripotent stem cell comprising introducing at least one of the transcription factors identified in Table 2, Table 3, Table 4, Table 5 or Table 6 into a target cell to produce an induced pluripotent stem cell.

The invention also provides a method of increasing the efficiency of reprogramming of a cell comprising introducing Obox6 into a target cell to produce an induced pluripotent stem cell.

The invention also provides a method of increasing the efficiency of reprogramming a cell comprising introducing at least one of the transcription factors identified in Table 2, Table 3, Table 4, Table 5 or Table 6 into a target cell to produce an induced pluripotent stem cell.

The invention also provides for an isolated induced pluripotent stem cell produced by the methods of the invention.

The invention also provides a method of treating a subject with a disease comprising administering to the subject a cell produced by differentiation of the induced pluripotent stem cell produced by the methods of the invention.

The invention also provides for a composition for producing an induced pluripotent stem cell comprising Obox6 or any of the factors identified in Table 2, Table 3, Table 4, Table 5 or Table 6 in combination with reprogramming media.

The invention also provides for use of Obox6 or one or more of the factors identified in Table 2, Table 3, Table 4, Table 5 or Table 6 for production of an induced pluripotent stem cell.

### Definitions

As used herein, “pluripotent” as it refers to a “pluripotent stem cell” means a cell with the developmental potential, under different conditions, to differentiate to cell types characteristic of all three germ cell layers, i.e., endoderm (e.g., gut tissue), mesoderm (including blood, muscle, and vessels), and ectoderm (such as skin and nerve). Pluripotent cell as used herein, includes a cell that can form a teratoma which includes tissues or cells of all three embryonic germ layers, or that resemble normal derivatives of all three embryonic germ layers (i.e., ectoderm, mesoderm, and endoderm). A pluripotent cell of the invention also means a cell that can form an embryoid body (EB) and express markers for all three germ layers including but not limited to the following: endoderm markers-AFP, FOXA2, GATA4; mesoderm markers-CD34, CDH2 (N-cadherin), COL2A1, GATA2, HAND1, PECAM1, RUNX1, RUNX2; and Ectoderm markers-ALDH1A1, COL1A1, NCAM1, PAX6, TUBB3 (Tuj1).

A pluripotent cell of the invention also means a human cell that expresses at least one of the following markers: SSEA3, SSEA4, Tra-1-81, Tra-1-60, Rexl, Oct4, Nanog, Sox2 as detected using methods known in the art. A pluripotent stem cell of the invention includes a cell that stains positive with alkaline phosphatase or Hoechst Stain.

In some embodiments, a pluripotent cell is termed an “undifferentiated cell.” Accordingly, the terms “pluripotency” or a “pluripotent state” as used herein refer to the developmental potential of a cell that provides the ability of the cell to differentiate into all three embryonic germ layers (endoderm, mesoderm and ectoderm). Those of skill in the art are aware of the embryonic germ layer or lineage that gives rise to a given cell type. A cell in a pluripotent state typically has the potential to divide in vitro for a long period of time, e.g., greater than one year or more than 30 passages.

As used herein, the term “induced pluripotent stem cells (iPSCs or “iPS cells)” refers to cells having similar properties to those of ES cells. In particular, an “iPSC” or “iPS cell” as used herein, includes an undifferentiated cell which is reprogrammed from somatic cells and have pluripotency and proliferation potency. However, this term is not to be construed as limiting in any sense, and should be construed to have its broadest meaning. As used herein, the term “pluripotent stem cell”, as it refers to the cell produced by the claimed methods is synonymous with the term “iPS”.

Obox6 and any of the other factors described herein can be used to generate induced pluripotent stem cells from differentiated adult somatic cells. In the preparation of induced pluripotent stem cells by using the factors of the present invention, types of cells to be reprogrammed are not particularly limited, and any kind of cells may be used. For example, matured somatic cells may be used, as well as somatic cells of an embryonic period. Other examples of cells capable of being generated into iPS cells and/or encompassed by the present invention include mammalian cells such as fibroblasts, mouse embryonic fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, pancreatic cells, hepatocytes, macrophages, monocytes, mononuclear cells, and gastric cells, including gastric epithelial cells. The cells can be embryonic, or adult somatic cells, differentiated cells, cells with an intact nuclear membrane, non-dividing cells, quiescent cells, terminally differentiated primary cells, and the like. The pluripotent or multipotent cells of the present invention possess the ability to differentiate into cells that have characteristic attributes and specialized functions, such as hair follicle cells, blood cells, heart cells, eye cells, skin cells, placental cells, pancreatic cells, or nerve cells. In particular, pluripotent cells of the invention can differentiate into multiple cell types including but not limited to: cells derived from the endoderm, mesoderm or ectoderm, including but not limited to cardiac cells, neural cells (for example, astrocytes and oligodendrocytes), hepatic cells (for example, pancreatic islet cells), osteogentic, muscle cells, epithelial cells, chondrocytes, adipocytes, placental cells, dendritic cells and, haematopoietic and retinal pigment epithelial (RPE) cells.

Induced pluripotent stem cells may express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; III-tubulin; -smooth muscle actin (-SMA); fibroblast growth factor 4 (Fgf4), Cripto, Daxl; zinc finger protein 296 (Zfp296); N-acetyltransferase-1 (Natl); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthll7; Sall4; undifferentiated embryonic cell transcription factor (Utfl); Rexl; p53; G3PDH; telomerase, including TERT; silent X chromosome genes; Dnmt3a; Dnmt3b; TRIM28; F-box containing protein 15 (Fbxl5); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; SV40 Large T Antigen; HPV16 E6; HPV16 E7, -catenin, and Bmil. Such cells can also be characterized by the down-regulation of markers characteristic of the differentiated cell from which the iPS cell is induced. For example, iPS cells derived from fibroblasts may be characterized by down-regulation of the fibroblast cell marker Thy1 and/or up-regulation of SSEA-1. It is understood that the present invention is not limited to those markers listed herein, and encompasses markers such as cell surface markers, antigens, and other gene products including ESTs, RNA (including microRNAs and antisense RNA), DNA (including genes and cDNAs), and portions thereof.

As used herein, “increases the efficiency” as it refers to the production of induced pluripotent stem cells, means an increase in the number of induced pluripotent stem cells that are produced, for example in the presence of Obox6 or one or more of the factors identified in Table 2, 3, 4, 5 or 6, as compared to the number of cells produced in the absence of Obox6 or one or more of the factors identified in Table 2, 3, 4, 5 or 6 under identical conditions. An increase in the number of induced pluripotent cells means an increase of at least 5%, for example, 5%, 10%, 15%, 20%, 25%, 30%, 35%, 40%, 45%, 50%, 55%, 60%, 65%, 70%, 75%, 80%, 85%, 90%, 95%, or 100% or more. An increase also means at least 5-fold more, for example, 5-fold, -fold, 20-fold, 30-fold, 40-fold, 50-fold, 60-fold, 70-fold, 80-fold, 90-fold, 100-fold, 500-fold, 1000-fold or more. Increases the efficiency also means decreasing the time required to produce an induced pluripotent stem cell, for example in the presence of Obox6 or one or more of the factors identified in Table 6, 7, 8, 9 or 10, as compared to the number of cells produced in the absence of Obox6 or one or more of the factors identified in Table 2, Table 3, Table 4, Table 5 and Table 6. In the presence of Obox6 or any one of the factors identified in Table 2, Table 3, Table 4, Table 5 and Table 6, an iPSC can be formed between 5 and 30 days, between 5 and 20 days, between 10 and 20 days, for example 10 days, 11 days, 12 days, 13 days, 14 days, 15 days, 16 days, 17 days, 18 days, 19 days or 20 days after the addition of Obox6 or one or more of the factors identified in Table 2, Table 3, Table 4, Table 5 and Table 6 or following induction of expression of Obox6 or or one or more of the factors identified in Table 2, Table 3, Table 4, Table 5 and Table 6.

Candidate transcriptional regulators to augment reprogramming efficiency include but are not limited to the transcription regulators presented in Tables 2, 3, 4, 5 and 6.

### Experimental Methods

### 1. Derivation of MEFs

Mouse embryonic fibroblasts (MEFs) were derived from E13.5 embryos with a mixed B6; 129 background. The cell line used in this study was homozygous for ROSA26-M2rtTA, homozygous for a polycistronic cassette carrying Pou5f1, Klf4, Sox2, and Myc at the Collal locus (18), and homozygous for an EGFP reporter under the control of the Pou5f1 promoter. Briefly, MEFs were isolated from E13.5 embryos resulting from timed-matings by removing the head, limbs, and internal organs under a dissecting microscope. The remaining tissue was finely minced using scalpels and dissociated by incubation at 37° C. for 10 minutes in trypsin-EDTA (Thermo Fisher Scientific). Dissociated cells were then plated in MEF medium containing DMEM (Thermo Fisher Scientific), supplemented with 10% fetal bovine serum (GE Healthcare Life Sciences), non-essential amino acids (Thermo Fisher Scientific), and GlutaMAX (Thermo Fisher Scientific). MEFs were cultured at 37° C. and 4% CO₂ and passaged until confluent. All procedures, including maintenance of animals, were performed according to a mouse protocol (2006N000104) approved by the MGH Subcommittee on Research Animal Care.

### 2. Reprogramming Assay

For the reprogramming assay, 20,000 low passage MEFs (no greater than 3-4 passages from isolation) were seeded in a 6-well plate. These cells were cultured at 37° C. and 5% CO₂ in reprogramming medium containing KnockOut DMEM (GIBCO), 10% knockout serum replacement (KSR, GIBCO), 10% fetal bovine serum (FBS, GIBCO), 1% GlutaMAX (Invitrogen), 1% nonessential amino acids (NEAA, Invitrogen), 0.055 mM 2-mercaptoethanol (Sigma), 1% penicillin-streptomycin (Invitrogen) and 1,000 U/ml leukemia inhibitory factor (LIF, Millipore). Day 0 medium was supplemented with 2 g/mL doxycycline Phase-1(Dox) to induce the polycistronic OKSM expression cassette. Medium was refreshed every other day. At day 8, doxycycline was withdrawn, and cells were transferred to either serum-free 2i medium containing 3 μM CHIR99021, 1 μM PD0325901, and LIF (Phase-2(2i)) (25) or maintained in reprogramming medium (Phase-2(serum)). Fresh medium was added every other day until the final time point on day 16. Oct4-EGFP positive iPSC colonies should start to appear on day 10, indicative of successful reprogramming of the endogenous Oct4 locus.

3. Sample collection

A total of 66,000 cells were collected from twelve time points over a period of 16 days in two different culture conditions. Single or duplicate samples were collected at day 0 (before and after Dox addition), 2, 4, 6, and 8 in Phase-1(Dox); day 9, 10, 11, 12, 16 in Phase-2(2i); and day 10, 12, 16 in Phase-2(serum). Cells were also collected from established iPSCs cell lines reprogrammed from the same MEFs, maintained either in Phase-2(2i) conditions or in Phase-2(serum) medium. For all time points, selected wells were trypsinized for 5 mins followed by inactivation of trypsin by addition of MEF medium. Cells were subsequently spun down and washed with 1×PBS supplemented with 0.1% bovine serum albumin. The cells were then passed through a 40 micron filter to remove cell debris and large clumps. Cell count was determined using Neubauer chamber hemocytometer to a final concentration of 1000 cells/1.

### 4. Single-Cell RNA Sequencing

Single-cell RNA-Seq libraries were generated from each time point using the 10× Genomics Chromium Controller Instrument (10× Genomics, Pleasanton, Calif.) and Chromium™ Single Cell 3′ Reagent Kits v1 (PN-120230, PN-120231, PN-120232) according to manufacturer's instructions. Reverse transcription and sample indexing were performed using the C1000 Touch Thermal cycler with 96-Deep Well Reaction Module. Briefly, the suspended cells were loaded on a Chromium controller Single-Cell Instrument to first generate single-cell Gel Bead-In-Emulsions (GEMs). After breaking the GEMs, the barcoded cDNA was then purified and amplified. The amplified barcoded cDNA was fragmented, Atailed and ligated with adaptors. Finally, PCR amplification was performed to enable sample indexing and enrichment of the 3′ RNA-Seq libraries. The final libraries were quantified using Thermo Fisher Qubit dsDNA HS Assay kit (Q32851) and the fragment size distribution of the libraries were determined using the Agilent 2100 BioAnalyzer High Sensitivity DNA kit (5067-4626). Pooled libraries were then sequenced using Illumina Sequencing By Synthesis (SBS) chemistry.

### 5. Lentivirus Vector Construction and Particle Production

To test whether transcription factors (TFs) improve late-stage reprogramming efficiency, lentiviral constructs for the top candidates Zfp42, and Obox6 were generated. cDNA for these factors were ordered from Origene (Zfp42-MG203929, and Obox6-MR215428) were cloned into the FUW Tet-On vector (Addgene, Plasmid #20323) using the Gibson Assembly (NEB, E2611S). Briefly, the cDNA for each TF was amplified and cloned into the backbone generated by removing Oct4 from the FUW-Teto-Oct4 vector. All vectors were verified by Sanger sequencing analysis. For lentivirus production, HEK293T cells were plated at a density of 2.6×10⁶ cells/well in a 10 cm dish. The cells were transfected with the lentiviral packaging vector and a TF-expressing vector at 70-80% growth confluency using the Fugene HD reagent (Promega E2311) according to the manufacturer's protocols. At 48 hours after transfection, the viral supernatant was collected, filtered and stored at −80° C. for future use.

6. Reprogramming Efficiency of Secondary MEFS Together with Individual TFs

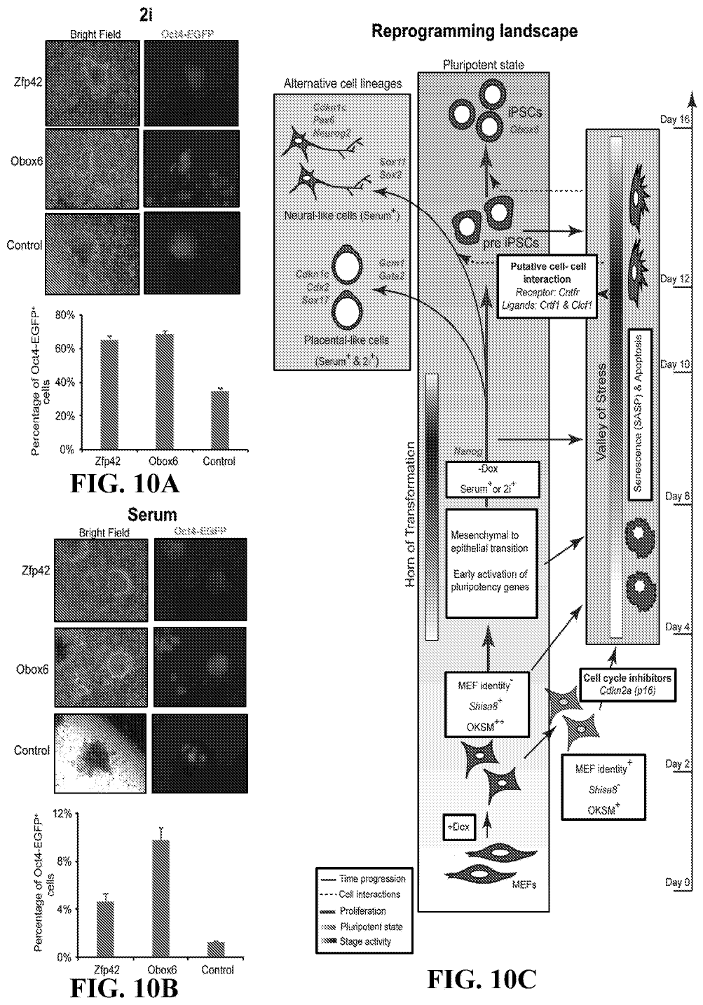

We sought to determine the ability of the candidate TFs to augment reprogramming efficiency in secondary MEFs; the use of secondary MEFs for reprogramming overcomes limitations associated with random lentiviral integration events at variable genomic locations. Briefly, secondary MEFs were plated at a concentration of 20,000 cells per well of a 6-well plate. Cells were infected with virus containing Zfp42, Obox6, or an empty vector and maintained in reprogramming medium as described above. At day 8 after induction, cells were switched to either Phase-2(2i) or Phase-2(serum). On day 16, reprogramming efficiency was quantified by measuring the levels of the EGFP reporter driven by the endogenous Oct4 promoter. FACS analyses was performed using the Beckman Coulter CytoFLEX S, and the percentage of Oct4-EGFP+ cells was determined. Triplicates were used to determine average and standard deviation (FIG. 10B).

7. Reprogramming Efficiency of Primary MEFS with Individual TFs and OKSM

In addition to demonstrating the ability of a TF to increase reprogramming efficiency in secondary MEFs, the performance of the TFs were independently tested in primary MEFs. To this end, lentiviral particles were generated from four distinct FUW-Teto vectors, containing Oct4, Sox2, Klf4, and Myc, MEFs from the background strain B6.Cg-Gt(ROSA)26Sortml(rtTA*M2)Jae/J×B6; 129S4-Pou5fltm2Jae/J were infected with these lentiviral particles, together with a lentivirus expressing tetracycline-inducible Zfp42, Obox6 or no insert. Infected cells were then induced with 2 μg/mL doxycycline in ESC reprogramming medium (day 0). At day 8 after induction, cells were switched to either Phase-2(2i) or Phase-2(serum). On day 16, the number of Oct4-EGFP+ colonies were counted using a fluorescence microscope. Triplicates for each condition used to determine average values and standard deviation.

### EXAMPLES

### Example 1

Computing Trajectories with Optimal Transport

As noted above, for any pair of time points we compute a transport plan that minimizes the expected cost of redistributing mass, subject to constraints involving a proliferation score (see Appendix 1 for a precise statement of the optimization problem). To compute these transport matrices, we need to specify a cost function, a proliferation function, and numerical values for the regularization parameters.

Cost functions: We tried several different cost functions based on squared Euclidean distance in different input spaces. Specifically, for cells with expression profiles x and y, given by two columns of the expression matrix E, we specify a cost function c(x, y)

c₁(x,y)=//x⁻−y⁻//² Expression space

c₂(x,y)=//ΛΦ₁₀₀(x)−ΛΦ₁₀₀(y)//² 100 dimensional diffusion component space

c₃(x,y)=//ΛΦ₂₀(x)−ΛΦ₂₀(y)//² 20 dimensional diffusion component space

The bar above x⁻, y⁻ denotes that we apply the truncation transform from section 2, and Φd is the Laplacian embedding from section 3. Note that Pd has the log transform x→\tilde{x} built-in. In the equations above, Λ is a diagonal matrix containing the eigenvalues of the Laplacian matrix, raised to the power 8. Hence c2 and c3 are both truncated versions of the diffusion distance D4(x, y) from (S5).

The cost function c3 was used to report the numerical values in the main text, and we computed separate transport maps for 2i and serum. Note that all the cost functions c1, c2, c3 give largely similar results.

Proliferation function: We estimate the relative growth rate for every cell using the proliferation signature displayed in FIG. 7D in the main text. To transform the proliferation score into an estimate of the growth rate (in doublings per day), we first observed that the proliferation score is bimodally distributed over the dataset. We transformed the proliferation score so that the two modes were mapped to a growth ratio of 2.5 per day (this means that over 1 day, a cell in the more proliferative group is expected to produce 2.5 times as many offspring as a cell in the non-proliferative group). However, note that we allow for some laxity in the prescribed growth rate (see supplemental figure on input vs implied proliferation).

Regularization parameters: We employed the following strategy to select the regularization pa-rameters X and E. The entropy parameter c controls the entropy of the transport map. An extremely large entropy parameter will give a maximally entropic transport map, and an extremely small entropy parameter will give a nearly deterministic transport map (but could also lead to numerical instability in the algorithm). We adjusted the entropy parameter until each cell transitions to between 10 and 50 percent of cells in the next time point, as measured by the Shannon diversity of the rows of the transport map.

The regularization parameter λ controls the fidelity of the constraints: as λ gets larger, the constraints become more stringent. We selected λ so that the marginals of the transport map are 95% correlated with the prescribed proliferation score.

Implementation: The scaling algorithm for unbalanced transport (S2) was implemented to compute optimal transport maps. This algorithm performs gradient ascent steps on the dual optimization problem. Because of the entropic regularization, these gradient ascent steps can be performed via diagonal matrix scalings. We implemented versions of the solver in both R and Python.

Experiments: Computational experiments were performed to evaluate the stability of our results to choice of cost function, regularization parameters, and subsampling the dataset.

The cluster-to-cluster origin were compared and fate tables for the different cost functions listed above, and consistent results were found. Moreover, the transport probabilities described above are all robust to choice of cost function.

A bootstrap analysis was performed on a batch of 100 subsamples consisting of 50% of the data from each time point. The variance in the cluster-to-cluster origin and fate tables is extremely small (see Table 7).

TABLE 7

| MEF.identity | Pluripotency | G1.S | G2.M | Cell.cycle | ER.stress | Epithelial.identity | ECM.rearrangement | Apoptosis | SASP | Neural.identity | Placental.identity | X.reactivation |
|---|---|---|---|---|---|---|---|---|---|---|---|---|
| Gm5571 | Rhox5 | Cdca7 | Cbx5 | Mcm4 | Nck2 | Cdh1 | Sulf1 | Ercc5 | Il6 | Vtn | 493343p14rik | Gm21950 |
| Rbfox2 | Tdgf1 | Mcm4 | Aurkb | Smc4 | Ankzf1 | Tgm1 | Col19a1 | Serpinb5 | Il7 | Ednrb | Esx1 | Gm21364 |
| Btbd19 | Utf1 | Mcm2 | Cks1b | Gtse1 | Dnajb2 | Cldn3 | Col3a1 | Inhbb | Il1a | Sox21 | Afap1 | Gm14346 |
| Actn1 | Mkrn1 | Rfc2 | Cks2 | Ttk | Rhbdd1 | Cldn4 | Col5a2 | Steap3 | Il1b | Zeb2 | Zfyve21 | Gm14345 |
| Gatad2a | Dppa5a | Ung | Hn1 | Rangap1 | Bcl2 | Cldn7 | Fn1 | Btg2 | Il13 | Hes5 | Erv3 | Gm14351 |
| Med6 | Upp1 | Mcm6 | Hmgb2 | Ccnb2 | Ubxn4 | Cldn11 | Ihh | Phlda3 | Il15 | Fabp7 | Atg12 | Gm3701 |
| Mex3a | Chchd10 | Rrm1 | Anp32e | Cenpa | Yod1 | Ocln | Col4a4 | Tnni1 | Cxcl15 | Sox1 | Las1l | Gm3706 |
| Ccdc80 | Klf2 | Slbp | Lbr | Cenpe | Ppp1r15b | Epcam | Col4a3 | Rgs16 | Cxcl1 | Neurod1 | Rbp1 | Gm14347 |
| Mex3c | Trap1a | Pcna | Tmpo | Cdca8 | Fam129a | Crb3 | Serpinb5 | Ier5 | Cxcl2 | Pax3 | Prl2b1 | Gm10921 |
| Sdpr | Mylpf | Atad2 | Top2a | Ckap2 | Edem3 | Krt8 | Fmod | Slc19a2 | Cxcl3 | Pax6 | Prl3d1 | Gm10922 |
| Pcdhb2 | 1700013H16Rik | Tipin | Tacc3 | Rad51 | Atf6 | Krt19 | Elf3 | Adck3 | Ccl8 | Cdh2 | Rnf2 | Gm3750 |
| Trim16 | AA467197 | Mcm5 | Tubb4b | Pcna | Ufc1 | Pkp3 | Lamc1 | Ephx1 | Ccl13 | Sox9 | Sct | Gm3763 |
| Obsl1 | Dhx16 | Uhrf1 | Ncapd2 | Ube2c | Atf3 | Dsp | Tnr | Ptpn14 | Ccl3 | Sox2 | Mrgprg | Mycs |
| Epha1 | Mt2 | Rpa2 | Rangap1 | Lbr | Man1b1 | Pkp1 | Dpt | Atf3 | Ccl20 | Id2 | Aa763515 | Gm14374 |
| Stx1b | Ube2a | Dtl | Cdk1 | Cenpf | Tor1a | | Ddr2 | Notch1 | Ccl16 | Hoxb1 | Tfpi | Nudt11 |
| Stau1 | Khdc3 | Prim1 | Smc4 | Birc5 | Hspa5 | | Olfml2b | Rxra | Ccl26 | Msx1 | Etos1 | AU022751 |
| Serpine1 | Pycard | Fen1 | Kif20b | Dtl | Dab2ip | | Tgfb2 | Ralgds | Csf2 | Msi1 | Slc5a6 | Nudt10 |
| Aa881470 | Hsp90aa1 | Hells | Cdca8 | Dscc1 | Nfe2l2 | | Itga8 | Ak1 | Csf3 | Msi2 | 1600025m17rik | Bmp15 |
| Col12a1 | Prrc1 | Gmnn | Ckap2 | Cbx5 | Dnajc10 | | Adamtsl2 | Stom | Ifng | Atoh1 | Gm9 | Shroom4 |
| 2010300f17rik | Hat1 | Pold3 | Ndc80 | Usp1 | Psmc3 | | Col5a1 | Ddb2 | Mif | Rbfox3 | Creb3l2 | Dgkk |
| Ccdc102a | Calcoco2 | Nasp | Dlgap5 | Hmmr | Creb3l1 | | Pomt1 | Cd82 | Areg | Map2 | Bbx | Ccnb3 |
| Nradd | Impa2 | Chaf1b | Hjurp | Wdr76 | Thbs1 | | Eng | Il1a | Ereg | Tubb3 | Prl3c1 | Akap4 |
| Pard6g | Saa3 | Gins2 | Ckap5 | Ung | Eif2ak4 | | Lmx1b | Pcna | Nrg1 | | Mta3 | Clcn5 |
| Ntn4 | Ooep | Pola1 | Bub1 | Hn1 | Chac1 | | Gsn | Bmp2 | Egf | | Prl2a1 | Usp27x |
| 5730471h19rik | Bnip3 | Msh2 | Ckap2l | Cks2 | Pdia3 | | Olfml2a | Trib3 | Fgf2 | | Gm9112 | Ppp1r3f |
| Sepn1 | Mt1 | Casp8ap2 | Ect2 | Kif20b | Bcl2l11 | | Creb3l1 | Procr | Hgf | | Afap1l2 | Ppp1r3fos |
| Peg12 | Asns | Cdc6 | Kif11 | Cdk1 | Ddrgk1 | | Hsd17b12 | Blcap | Fgf7 | | Erlin2 | Foxp3 |
| Dpysl3 | Aldoa | Ubr7 | Birc5 | Slbp | Tmx4 | | Wt1 | Ada | Vegfa | | Pard3 | Ccdc22 |
| 1110012d08rik | Tdh | Ccne2 | Cdca2 | Aurkb | Trib3 | | Grem1 | Fgf13 | Ang | | Aif1l | Cacna1f |
| Akt1 | Gjb3 | Wdr76 | Nuf2 | Kif11 | H13 | | Spint1 | Irak1 | Kitl | | Dmrtc1a | Syp |
| Zfp286 | Rbpms2 | Tyms | Cdca3 | Cks1b | Edem2 | | Cst3 | Tspyl2 | Cxcl12 | | 4932442l08rik | Gm14703 |
| Ubap2l | Prps1 | Cdc45 | Nusap1 | Blm | Cebpb | | Fkbp1a | Sat1 | Pigf | | GJb2 | Prickle3 |
| Samd4 | Fam25c | Clspn | Ttk | Msh2 | Ptpn1 | | Mmp9 | Zmat3 | Igfbp2 | | Gjb5 | Plp2 |
| Phc2 | Eif2s2 | Rrm2 | Aurka | Gas2l3 | Vapb | | Sulf2 | Hspa4l | Igfbp3 | | Slco5a1 | Magix |
| Mcam | Cenpm | Dscc1 | Mki67 | Tyms | Srpx | | Atp7a | Slc7a11 | Igfbp4 | | Wdr61 | Gpkow |
| Pla2g4c | Nanog | Rad51 | Fam64a | HjurP | Aifm1 | | Nox1 | Tm4sf1 | Igfbp6 | | Kitl | Wdr45 |
| Fzd7 | Ndufa4l2 | Usp1 | Ccnb2 | Hells | Ubqln2 | | Col4a6 | Rap2b | Igfbp7 | | 9430027b09rik | RP23-109E24.10 |
| Pappa | Syce2 | Exo1 | Tpx2 | Prim1 | Mbtps2 | | Prdx4 | Fbxw7 | Mmp1 | | Tfrc | Praf2 |
| Ptk7 | Gm13251 | Blm | Hjurp | Uhrf1 | Usp13 | | Gpm6b | S100a4 | Mmp3 | | Slc6a2 | Ccdc120 |
| Nuak1 | Taf7 | Rad51ap1 | Anln | Ndc80 | Ufm1 | | Egfl6 | S100a10 | Mmp10 | | Wdr45 | Tfe3 |
| Il17rd | Nudt4 | Mlf1ip | Kif2c | Mcm6 | Serp1 | | Postn | Txnip | Mmp12 | | Zxda | Gripap1 |
| Ptk2 | Cox5a | E2f8 | Cenpe | Rrm1 | Creb3l4 | | Rxfp1 | Nhlh2 | Mmp13 | | Prdx4 | Kcnd1 |
| Ehd2 | Sod2 | Brip1 | Gtsel | Mlf1ip | Tmem67 | | Sfrp2 | Dnttip2 | Mmp14 | | Fam122b | Otud5 |
| Lats2 | S100a13 | | Kif23 | Top2a | Ufl1 | | Hapln2 | Clca2 | Timp2 | | Zxdb | Pim2 |
| Hspg2 | Fkbp6 | | Cdc20 | Hmgb2 | Ube2j1 | | Ctss | Wwp1 | Serpine1 | | Zxdc | Slc35a2 |
| 4930456g14rik | Rhox9 | | Ube2c | Ccne2 | Vcp | | Adamtsl4 | Klf4 | Serpinb2 | | Pip5k1a | Pqbp1 |
| 4930429b21rik | Gdf3 | | Cenpf | G2e3 | Creb3 | | St7l | Ikbkap | Plat | | Plac1 | Timm17b |
| Rps20 | 2700094K13Rik | | Cenpa | Tmpo | Sec61b | | Col11a1 | Cdkn2a | Plau | | Igf2as | Gm10491 |
| Vgll3 | Fmr1nb | | Hmmr | Nusap1 | Erp44 | | Npnt | Cdkn2b | Ctsb | | Usp9x | Gm10490 |
| Prr15 | Hmgn2 | | Ctcf | Ncapd2 | Al314180 | | Cyr61 | Jun | Icam1 | | Psg28 | Pcsk1n |
| Fbxl7 | Ubald2 | | Psrc1 | Mcm2 | Jun | | B4galt1 | Slc35d1 | Icam3 | | Bmp8b | Eras |
| Maged2 | Lactb2 | | Cdc25c | Kif2c | Casp9 | | Reck | Plk3 | Tnfrsf11b | | Fn1 | Hdac6 |
| Galntl4 | Folr1 | | Nek2 | Cdca2 | Fbxo6 | | Tgfbr1 | Rnf19b | Tnfrsf1a | | Psg23 | Gata1 |
| Pdgfc | Gm7325 | | Gas2l3 | Nasp | Fbxo2 | | Col27a1 | Sfn | Tnfrsf1b | | Bmp8a | Glod5 |
| Tmtc4 | Agtrap | | G2e3 | Gmnn | Ube4b | | P3h1 | Fuca1 | Tnfrsf10b | | Psg21 | Gm14820 |
| Tmtc3 | Spp1 | | | Cdc6 | Ube2j2 | | Hspg2 | Epha2 | Fas | | Dusp9 | Suv39h1 |
| Lpar4 | Hells | | | Pold3 | Psmc2 | | Vwa1 | Wrap73 | Plaur | | H19 | Was |
| Pcdh19 | Dppa4 | | | Ckap2l | Tmub1 | | Dnajb6 | Mxd4 | Il6st | | Tmem37 | Wdr13 |
| Eda2r | Gabarapl2 | | | Fam64a | Tmem129 | | Emilin1 | Rchy1 | Egfr | | Mmp15 | Rbm3 |
| Pcdh18 | Rhox6 | | | Ubr7 | Wfs1 | | Mpv17 | Iscu | Fn1 | | Fam101b | Rbm3os |
| Gpr176 | Rhox1 | | | Fen1 | Ube2k | | Apbb2 | Triap1 | | | Phf16 | Tbc1d25 |
| Loc100503471 | Cdc5l | | | Bub1 | Tbl2 | | Pdgfra | Prkab1 | | | 4930422n03rik | Ebp |
| Mical2 | Tex19.1 | | | Brip1 | Get4 | | Ambn | Trafd1 | | | Ada | Porcn |
| Dzip1l | Trim28 | | | Atad2 | Bhlha15 | | Dmp1 | Pom121 | | | Mmp1a | Ftsj1 |
| Hoxc6 | Atp5g1 | | | Psrc1 | Creb3l2 | | Ibsp | Pdgfa | | | Gpr126 | Slc38a5 |
| Hoxc5 | Sox2 | | | Rrm2 | Pdia4 | | Tfip11 | Gadd45a | | | Arf2 | Ssxb10 |
| Mettl4-ps1 | Jam2 | | | Tipin | Eif2ak3 | | Eln | Vamp8 | | | Tinagl1 | Ssxb9 |
| Sec63 | Fkbp3 | | | Casp8ap2 | Rnf103 | | Plod3 | Retsat | | | Mfi2 | Ssxb1 |
| Ikbip | Cox7b | | | Tubb4b | Aup1 | | Col1a2 | Tprkb | | | Rpn2 | Ssxb2 |
| Tsc22d2 | Ash2l | | | Kif23 | Itpr1 | | Ndnf | Tgfa | | | Abhd2 | Gm14459 |
| 2310076g05rik | Dut | | | Exo1 | Edem1 | | Vhl | Mxd1 | | | Hrct1 | Ssxb6 |
| Anxa6 | Dtymk | | | Rfc2 | Bbc3 | | Mfap5 | Sec61a1 | | | Adm | Ssxb3 |
| Nfatc4 | Gpx4 | | | Pola1 | Psmc4 | | Ercc2 | Xpc | | | Abhd6 | Ssxb8 |
| Fn1 | Eif4ebp1 | | | Mki67 | Bax | | Bcl3 | Ccnd2 | | | Slc7a1 | Ssx9 |
| Wnt9a | Morc1 | | | Tpx2 | Ppp1r15a | | Tgfb1 | H2afj | | | Tead4 | Ssxb5 |
| Sorcs2 | Fabp3 | | | Aurka | Vimp | | Mia | Ldhb | | | Mbnl3 | Gm6592 |
| Tmeff1 | Zfp428 | | | Anln | Rnf121 | | Spint2 | Lrmp | | | Gpr1 | Gm5751 |
| C79491 | Aqp3 | | | Chaf1b | Anks4b | | Aplp1 | Tm7sf3 | | | 2900057e15rik | B630019K06Rik |
| Crlf1 | Grhpr | | | Hjurp | Ern2 | | Hpn | Tgfb1 | | | Ldoc1 | Fthl17b |
| 2610034e01rik | Higd1a | | | Tacc3 | Atp2a1 | | Klk4 | Sertad3 | | | Adam19 | Fthl17c |
| Gjd4 | Rpp25 | | | Mcm5 | Brsk2 | | Acan | Cebpa | | | Rybp | Fthl17d |
| Ccng1 | Rbpms | | | Anp32e | Ins2 | | Serpinh1 | Klk8 | | | Col4a1 | Fthl17e |
| Gpr124 | Mmp3 | | | Dlgap5 | Ccnd1 | | Apbb1 | Bax | | | Fndc3c1 | Fthl17f |
| Fibin | Apobec3 | | | Ect2 | Map3k5 | | Ilk | Ppp1r15a | | | Col4a2 | 4930402K13Rik |
| 8030476l19rik | Spc24 | | | Nuf2 | Nrbf2 | | Ric8 | Rpl18 | | | 4930502el8rik | Lancl3 |
| Ddr2 | Xlr3a | | | Cdc45 | Derl3 | | Muc5ac | Aen | | | Pkn2 | Gm14862 |
| Arf4 | Rec114 | | | Ckap5 | Ube2g2 | | Ctgf | Rrp8 | | | Rlim | Xk |
| Ptprs | Mtf2 | | | Ctcf | Tmem259 | | Nr2e1 | Ccp110 | | | 1600015i10rik | 1700012L04Rik |
| Sprr2k | Snrpn | | | Clspn | Creb3l3 | | Nepn | Nupr1 | | | Afp | Gm14501 |
| Adm | Gm13580 | | | Cdca7 | Hsp90b1 | | P4ha1 | Ptpre | | | Tmem140 | Cybb |
| A830029e22rik | Gmnn | | | Cdca3 | Apaf1 | | Spock2 | Hras | | | Fstl3 | Gm5132 |
| 9230114k14rik | Chmp4c | | | Rpa2 | Ifng | | Adamts14 | Eps8l2 | | | Ing4 | Dynlt3 |
| Extl3 | Hsf2bp | | | Gins2 | Os9 | | Mmp11 | Ctsd | | | Taf7l | Hypm |
| Mecom | Polr2e | | | E2f8 | Ddit3 | | Col18a1 | Cd81 | | | Sult1e1 | 4930557A04Rik |
| Qsox1 | Blvrb | | | Cdc25c | Erlin2 | | Myf5 | Perp | | | Olr1 | Sytl5 |
| Tead1 | Ldhb | | | Nek2 | Ppp2cb | | Col4a1 | Rps12 | | | 2610019f03rik | Srpx |
| Snx7 | Apoc1 | | | Cdc20 | Ubxn8 | | Csgalnact1 | Tpd52l1 | | | F11 | Rpgr |
| Cdkl4 | Syngr1 | | | Rad51ap1 | Casp3 | | Comp | Sesn1 | | | Fbxw8 | Otc |
| Cdkn2a | Bex1 | | | | Pik3r2 | | Gfod2 | Foxo3 | | | Sema4c | Tspan7 |
| Cdkn2b | Nr2c2ap | | | | Amfr | | Has3 | Ddit4 | | | Ctnnbip1 | Gm10489 |
| Ccnyl1 | | | | | Herpud1 | | Atxn1l | Zfp365 | | | Tfpi2 | Mid1ip1 |
| Tubb2a-ps2 | | | | | Aars | | Crispld2 | Prmt2 | | | Zbtb10 | Gm14493 |
| Aen | | | | | Selk | | Foxf1 | Mknk2 | | | Mitf | Gm14483 |
| Farp1 | | | | | Ero1l | | Foxc2 | Dram1 | | | Gpr50 | Gm14474 |
| 4930402h24rik | | | | | Psmc6 | | Agt | Apaf1 | | | Hic2 | Gm14477 |
| Sh3rf3 | | | | | Trim13 | | Exoc8 | Btg1 | | | Tpbpb | Gm14476 |
| Adam19 | | | | | Dnajc3 | | Ero1l | Mdm2 | | | Slc9a6 | Gm14484 |
| Ddb1 | | | | | Casp4 | | Lgals3 | Ddit3 | | | Prl7d1 | Gm14479 |
| Cttn | | | | | Casp12 | | Ripk3 | Gls2 | | | Tpbpa | Gm14482 |
| 9230112e08rik | | | | | Scamp5 | | Loxl2 | Dgka | | | Slco2a1 | Gm14478 |
| Dbn1 | | | | | Pml | | Lcp1 | Cdkn2aip | | | Pkp2 | Gm14475 |
| Fyttd1 | | | | | Parp16 | | Mmp13 | Hmox1 | | | 9630050e16rik | Gm4906 |
| Lrrc15 | | | | | Nck1 | | Mmp20 | Rrad | | | Pvrl2 | Bcor |
| Fkbp10 | | | | | Uba5 | | Col5a3 | Cdh13 | | | Zfp568 | Gm14635 |
| Trub1 | | | | | Usp19 | | Smarca4 | Osgin1 | | | Vtcn1 | Atp6ap2 |
| Zdhhc20 | | | | | Stt3b | | Aplp2 | Cgrrf1 | | | Il6ra | 1810030O07Rik |
| Ston1 | | | | | Rnf185 | | Mpzl3 | Abhd4 | | | Foxo4 | Med14 |
| Hoxd13 | | | | | Xbp1 | | Thsd4 | Kif13b | | | Hsp90b1 | Usp9x |
| Nudt6 | | | | | Erlec1 | | Anxa2 | Rb1 | | | Prl7c1 | 2010308F09Rik |
| Hoxd12 | | | | | Stc2 | | Myo1e | Nudt15 | | | Prl6a1 | Ddx3x |
| Prss23 | | | | | Trp53 | | Nphp3 | Tsc22d1 | | | Cdh5 | Nyx |
| 9430030n17rik | | | | | Alox15 | | Dag1 | Casp1 | | | Fgd6 | Cask |
| Arntl2 | | | | | Derl2 | | Lamb2 | St14 | | | Cysltr2 | Gpr34 |
| Sh3rfl | | | | | Trim25 | | Kif9 | Ei24 | | | Rhox6 | Gpr82 |
| Mrc2 | | | | | Cdk5rap3 | | Sh3pxd2b | Vwa5a | | | Cdh3 | Gm5382 |
| Mdh1 | | | | | Ccdc47 | | Adamts2 | Zbtb16 | | | Spp2 | Gm14505 |
| Rictor | | | | | Psmc5 | | Wnt3a | Rps27l | | | Zim1 | Drr1 |
| Map4k5 | | | | | Ern1 | | Mfap4 | Mapkapk3 | | | Flnb | Cypt1 |
| Plcl1 | | | | | Nploc4 | | Serpinf2 | Ip6k2 | | | Rbbp7 | Maoa |
| Sept11 | | | | | P4hb | | Vtn | Tcn2 | | | Map3k7 | Maob |
| Ryk | | | | | Txndc5 | | Nf1 | Lif | | | Rhox9 | Ndp |
| Tgfb3 | | | | | Faf2 | | Col1a1 | Upp1 | | | Whsc1l1 | Efhc2 |
| Ube2i | | | | | Ubqln1 | | Ramp2 | Ccng1 | | | Slc38a1 | Fundc1 |
| Tgfb2 | | | | | Atg10 | | Gfap | Cyfip2 | | | 1600012p17rik | Dusp21 |
| Zfp319 | | | | | Thbs4 | | Sox9 | Gnb2l1 | | | Adra2b | Kdm6a |
| Gm10399 | | | | | Col4a3bp | | Ero1lb | Hint1 | | | Pgf | 4930578C19Rik |
| Fbxo17 | | | | | Pik3r1 | | Nid1 | Gm2a | | | 1200009i06rik | Gm26652 |
| Wnt5a | | | | | Pdia6 | | Foxf2 | Hist3h2a | | | Mfsd7c | BC049702 |
| Crim1 | | | | | Dnajb9 | | Foxc1 | Alox8 | | | Esam | Chst7 |
| Mid1 | | | | | Tmx1 | | Ripk1 | Trp53 | | | Gpr107 | Slc9a7 |
| Disp1 | | | | | Jkamp | | Tfap2a | Tax1bp3 | | | Au015791 | Rp2 |
| Ubox5 | | | | | Sel1l | | Ecm2 | Traf | | | Arhgap8 | Jade3 |
| St7l | | | | | Psmc1 | | B4galt7 | Cdk5r1 | | | Ankrd17 | Rgn |
| Col5a2 | | | | | Atxn3 | | Tgfbi | Ppm1d | | | Cul7 | Ndufb11 |
| Axl | | | | | Derl1 | | Pxdn | Rad51c | | | 2310067p03rik | Rbm10 |
| Col5a1 | | | | | Rnf139 | | Smoc1 | Tob1 | | | Irs3 | Uba1 |
| Zyx | | | | | Foxred2 | | Ltbp2 | Krt17 | | | Prl5a1 | Cdk16 |
| Ror2 | | | | | Pla2g6 | | Flrt2 | Hexim1 | | | Fntb | Usp11 |
| Wdfy3 | | | | | Atf4 | | Fbln5 | Fdxr | | | Tceanc | Araf |
| Amotl2 | | | | | Ep300 | | Egflam | Itgb4 | | | Lepr | Syn1 |
| Yap1 | | | | | Tmbim6 | | Tnfrsf11b | Sphk1 | | | Tnfrsf9 | Timp1 |
| Phldb2 | | | | | Txndc11 | | Col14a1 | Rhbdf2 | | | Papola | Cfp |
| 6330562c20rik | | | | | Sdf2l1 | | Has2 | Baiap2 | | | Srd5a1 | Elk1 |
| Ctnnd1 | | | | | Ufd1l | | Ptk2 | Dcxr | | | C1qtnf1 | Uxt |
| Rock2 | | | | | Eif2b5 | | Scx | Hist1h1c | | | Slc38a4 | Zfp182 |
| Masp1 | | | | | Nrros | | Fbln1 | Ninj1 | | | Angpt4 | Spaca5 |
| Pvt1 | | | | | Pdia5 | | Adamts20 | Nol8 | | | Ctla2a | Zfp300 |
| Tnc | | | | | Gsk3b | | Col2a1 | F2r | | | 9930012k11rik | Ssxa1 |
| Fbln2 | | | | | Park2 | | Myh11 | Ankra2 | | | Mical3 | Gm21876 |
| Hdlbp | | | | | Stub1 | | Ccdc80 | Plk2 | | | Apoa4 | 4930453H23Rik |
| Atp10a | | | | | Pdia2 | | Abi3bp | Sdc1 | | | Cul4b | Gm6938 |
| Loxl1 | | | | | Crebrf | | App | Gpx2 | | | 3632454l22rik | Gm26593 |
| Loxl2 | | | | | Bak1 | | Serac1 | Zfp36l1 | | | Psg-ps1 | Agtr2 |
| Fbln5 | | | | | Rnf5 | | Plg | Fos | | | Lcor | Slc6a14 |
| Ctgf | | | | | Atf6b | | Smoc2 | Ccnk | | | Tnfrsf22 | Gm28269 |
| Efnb2 | | | | | Bag6 | | Has1 | Jag2 | | | Tnfrsf23 | Gm28268 |
| Rxra | | | | | Flot1 | | Noxo1 | Ndrg1 | | | Sos1 | Klhl13 |
| Ccnd2 | | | | | Eif2ak2 | | Col11a2 | Pmm1 | | | Dlx3 | Wdr44 |
| Gpc2 | | | | | Pmaip1 | | Tnxb | Plxnb2 | | | Ippk | Gm4907 |
| Ntf3 | | | | | Tmx3 | | Tnf | Vdr | | | Htr2b | Gm4985 |
| Kif5b | | | | | Syvn1 | | 2300002M23Rik | Csrnp2 | | | Dusp16 | Gm27192 |
| Slit2 | | | | | Erlin1 | | Flot1 | Acvr1b | | | Cdc73 | Gm5934 |
| Tpm1 | | | | | | | Hsp90ab1 | Sp1 | | | 1700025g04rik | Gm4297 |
| Gpc4 | | | | | | | Wash1 | Abat | | | Prl4a1 | Gm5935 |
| Flnb | | | | | | | Vit | Socs1 | | | Zfp655 | Gm5169 |
| 4930555b11rik | | | | | | | Cyp1b1 | Abcc5 | | | Slcl3a4 | Gm1993 |
| Flnc | | | | | | | Fshr | Trp63 | | | Ceacam14 | E330010L02Rik |
| C76332 | | | | | | | Mkx | Fam162a | | | Ceacam15 | Gm5168 |
| Capn2 | | | | | | | Lox | App | | | Trap1a | Gm2012 |
| Phlda3 | | | | | | | Hpse2 | Rab40c | | | Ceacam12 | Gm2030 |
| Map3k7 | | | | | | | Kazald1 | Bak1 | | | Gm16515 | Slx |
| Myh10 | | | | | | | Nfkb2 | Def6 | | | Ceacam13 | Gm14525 |
| D18ertd653e | | | | | | | | Cdkn1a | | | 4930447f24rik | Gm6121 |
| Stox2 | | | | | | | | Tap1 | | | Gzmd | Gm10230 |
| Igf2r | | | | | | | | Ier3 | | | Foxj2 | Gm2101 |
| D15ertd621e | | | | | | | | Polh | | | Fbxl19 | Gm10058 |
| Arid5b | | | | | | | | Ccnd3 | | | Gzmc | Gm2117 |
| Tnfrsf10b | | | | | | | | Hbegf | | | Gzmf | Gm4836 |
| 2610011e03rik | | | | | | | | Hdac3 | | | Gzme | Gm10147 |
| Ckap4 | | | | | | | | Rad9a | | | Gzmg | Gm2165 |
| Efna2 | | | | | | | | Ctsf | | | Patl2 | Gm10096 |
| Picalm | | | | | | | | Slc3a2 | | | 3830417a13rik | Gm2200 |
| Cdh10 | | | | | | | | Fas | | | Tspan14 | Gm26818 |
| Ddah1 | | | | | | | | | | | Hand1 | Gm3669 |
| Uba3 | | | | | | | | | | | Atxn10 | Gm10488 |
| 0610038b21rik | | | | | | | | | | | Mgat4a | E330016L19Rik |
| Gemin7 | | | | | | | | | | | Unc50 | Gm14632 |
| Uba1 | | | | | | | | | | | Il2rb | Gm7437 |
| Fbn1 | | | | | | | | | | | Ceacam11 | Gm14974 |
| Lhx9 | | | | | | | | | | | Plekhg1 | Gm10487 |
| Eif4g2 | | | | | | | | | | | Prl3b1 | Gm21447 |
| Vcl | | | | | | | | | | | Folr1 | Spin2f |
| Bcl2l2 | | | | | | | | | | | A830080d01rik | Gm2784 |
| Cd276 | | | | | | | | | | | Blzf1 | Gm2777 |
| Lrrc58 | | | | | | | | | | | Zfp667 | Gm21883 |
| Wwc2 | | | | | | | | | | | Flt1 | Spin2e |
| Lpp | | | | | | | | | | | Usp27x | Gm21608 |
| Arl1 | | | | | | | | | | | Hdac4 | Gm21637 |
| Ltbp1 | | | | | | | | | | | Itgb3 | Gm21645 |
| Ltbp2 | | | | | | | | | | | Sri | Gm2799 |
| Wisp1 | | | | | | | | | | | Sema3f | Gmcl1l |
| Igf1r | | | | | | | | | | | Prl3a1 | Gm5926 |
| Rhobtb3 | | | | | | | | | | | Bahd1 | Gm21951 |
| Fam198b | | | | | | | | | | | Sin3b | Gm21657 |
| Cnn2 | | | | | | | | | | | Gm2a | Gm21789 |
| Glipr2 | | | | | | | | | | | Serpinb9g | Gm2825 |
| Syde1 | | | | | | | | | | | Bend4 | Spin2-ps6 |
| Hhat | | | | | | | | | | | Bend5 | Gm2863 |
| Zmat3 | | | | | | | | | | | Serpinb9b | Gm2854 |
| Cald1 | | | | | | | | | | | Serpinb9c | Gm2913 |
| Pmepa1 | | | | | | | | | | | Serpinb9d | Gm2927 |
| E130112l23rik | | | | | | | | | | | Plekhh1 | Gm2933 |
| Bag2 | | | | | | | | | | | 2210011c24rik | Gm2964 |
| Zfp583 | | | | | | | | | | | Cd320 | Gm21870 |
| Pibf1 | | | | | | | | | | | Ccnjl | Gm21681 |
| Pmaip1 | | | | | | | | | | | Entpd2 | Spin2g |
| A130022j15rik | | | | | | | | | | | Il1r2 | Gm21699 |
| Bcl9l | | | | | | | | | | | Sfmbt2 | Gm14552 |
| Cpa6 | | | | | | | | | | | 1700011m02rik | Gm10486 |
| D13ertd787e | | | | | | | | | | | Plekha7 | Gm2309 |
| Pabpc4l | | | | | | | | | | | Sfrp5 | Gm14553 |
| Zfhx3 | | | | | | | | | | | Ppp1r3f | Gm14819 |
| Itga5 | | | | | | | | | | | Obsl1 | Dock11 |
| Txnrd1 | | | | | | | | | | | Slc23a3 | Il13ra1 |
| Htr1b | | | | | | | | | | | Tmem87b | Zcchc12 |
| Hmga2 | | | | | | | | | | | Epas1 | Lonrf3 |
| Sept2 | | | | | | | | | | | Ccdc68 | Gm6268 |
| Lamb1 | | | | | | | | | | | Kdelr2 | Gm14569 |
| Zfp518b | | | | | | | | | | | Pramef12 | Pgrmc1 |
| Parva | | | | | | | | | | | Lrp8 | Akap17b |
| Gulp1 | | | | | | | | | | | Pard6b | Slc25a43 |
| Shank1 | | | | | | | | | | | Peg10 | Slc25a5 |
| Bmp1 | | | | | | | | | | | N4bp2 | Gm14549 |
| Akt1s1 | | | | | | | | | | | Pla2g4e | 2310010G23Rik |
| Itga9 | | | | | | | | | | | Fam78b | C330007P06Rik |
| Abcc1 | | | | | | | | | | | Arrdc3 | Ube2a |
| Eda | | | | | | | | | | | Pla2g4d | Nkrf |
| B4galt2 | | | | | | | | | | | Rassf8 | Gm15008 |
| Nid1 | | | | | | | | | | | Au015836 | Sept6 |
| Ncam1 | | | | | | | | | | | Csnk1e | Sowahd |
| Shc2 | | | | | | | | | | | Stag1 | Rpl39 |
| Uba6 | | | | | | | | | | | Vnn1 | Upf3b |
| Tradd | | | | | | | | | | | Tchhl1 | Nkap |
| Rtel1 | | | | | | | | | | | Pla1a | Akap14 |
| Bicd2 | | | | | | | | | | | Slc45a4 | Ndufa1 |
| Adamts12 | | | | | | | | | | | Tex264 | Rnf113a1 |
| Hs2st1 | | | | | | | | | | | Pcdh12 | Gm9 |
| D10ertd610e | | | | | | | | | | | Ctr9 | Rhox1 |
| Cyr61 | | | | | | | | | | | Ccr1l1 | Rhox2a |
| Gtf3cl | | | | | | | | | | | Htatsf1 | Rhox3a |
| Lbh | | | | | | | | | | | 9030409g11rik | Rhox4a |
| Krt33b | | | | | | | | | | | Tspan9 | Rhox3a2 |
| Gm6607 | | | | | | | | | | | Rassf6 | Rhox4a2 |
| D3wsu167e | | | | | | | | | | | 4631402f24rik | Rhox2b |
| Zc3h7b | | | | | | | | | | | A2m | Rhox4b |
| 7630403g23rik | | | | | | | | | | | Rimklb | Rhox2c |
| Tnpo2 | | | | | | | | | | | Loc100504569 | Rhox3c |
| Cep170 | | | | | | | | | | | Apob | Rhox4c |
| Pdlim5 | | | | | | | | | | | Tmem150a | Rhox2d |
| Pdlim7 | | | | | | | | | | | 9130404d08rik | Rhox4d |
| Cad | | | | | | | | | | | Prl8a6 | Rhox2e |
| Unc5b | | | | | | | | | | | Cts6 | Rhox3e |
| 2410018l13rik | | | | | | | | | | | Prl8a8 | Rhox4e |
| Loc100216343 | | | | | | | | | | | Prl8a9 | Rhox2f |
| Glrx3 | | | | | | | | | | | Cts3 | Rhox3f |
| Kctd5 | | | | | | | | | | | Krt18 | Rhox4f |
| Loc269472 | | | | | | | | | | | Nrn1l | Rhox3g |
| Myo1c | | | | | | | | | | | Sfi1 | Rhox2g |
| 4930562c15rik | | | | | | | | | | | Tlr5 | Rhox4g |
| Tll1 | | | | | | | | | | | Rhou | Rhox3h |
| Sema3a | | | | | | | | | | | Arhgef6 | Rhox2h |
| Itgb1 | | | | | | | | | | | Tmem185b | Rhox5 |
| Nxn | | | | | | | | | | | Tram2 | Rhox6 |
| Tmem41b | | | | | | | | | | | Cited1 | Rhox7a |
| Sec23a | | | | | | | | | | | Cited2 | Rhox8 |
| Gm22 | | | | | | | | | | | Zfand2a | Rhox7b |
| Itgb5 | | | | | | | | | | | Krt25 | Rhox9 |
| Dysf | | | | | | | | | | | Klk4 | Btg1-ps1 |
| Thbs1 | | | | | | | | | | | Tnfrsf11b | Btg1-ps2 |
| Bc022687 | | | | | | | | | | | 2010204k13rik | Rhox10 |
| Dnm3os | | | | | | | | | | | Tor1aip2 | Rhox11 |
| Rnd3 | | | | | | | | | | | Fmr1nb | Rhox12 |
| Pik3c2a | | | | | | | | | | | Ctsr | Rhoxl3 |
| 2810008m24rik | | | | | | | | | | | Ctsq | Zbtb33 |
| Spred3 | | | | | | | | | | | Prl8a2 | Tmem255a |
| Senp5 | | | | | | | | | | | Ctsm | Atp1b4 |
| Arl13b | | | | | | | | | | | Prl8al | Lamp2 |
| Polr2e | | | | | | | | | | | Ctsj | Gm7598 |
| Itgav | | | | | | | | | | | Mpzl1 | Cul4b |
| Igf2bp3 | | | | | | | | | | | Stra6 | Mcts1 |
| | | | | | | | | | | | Bcap31 | Clgalt1c1 |
| | | | | | | | | | | | Creg1 | Gm14565 |
| | | | | | | | | | | | Tcfap2c | 603049 |
| | | | | | | | | | | | | 8E09Rik |
| | | | | | | | | | | | Prl7b1 | Cypt15 |
| | | | | | | | | | | | Ghrh | Cypt14 |
| | | | | | | | | | | | 4930486l24rik | Gria3 |
| | | | | | | | | | | | Neurog2 | Thoc2 |
| | | | | | | | | | | | 5430425j12rik | Xiap |
| | | | | | | | | | | | Prl7a1 | Stag2 |
| | | | | | | | | | | | Prl7a2 | Gm43337 |
| | | | | | | | | | | | Mir1199 | Sh2d1a |
| | | | | | | | | | | | Tbc1d10a | Tenm1 |
| | | | | | | | | | | | Ralbp1 | Gm362 |
| | | | | | | | | | | | Pdgfra | Dcaf12l2 |
| | | | | | | | | | | | Morc4 | Dcaf12l1 |
| | | | | | | | | | | | Rarres2 | Prr32 |
| | | | | | | | | | | | Arid3a | 4930515L19Rik |
| | | | | | | | | | | | Lifr | Actrt1 |
| | | | | | | | | | | | Shisa3 | Gm29242 |
| | | | | | | | | | | | Uevld | Smarca1 |
| | | | | | | | | | | | Scnn1b | Ocrl |
| | | | | | | | | | | | Dnajb12 | Apln |
| | | | | | | | | | | | Brwd3 | Xpnpep2 |
| | | | | | | | | | | | Hhipl1 | Sash3 |
| | | | | | | | | | | | Fbln7 | Zdhhc9 |
| | | | | | | | | | | | Masp1 | Utp14a |
| | | | | | | | | | | | Nrk | 9530027J09Rik |
| | | | | | | | | | | | Pvr | Bcorl1 |
| | | | | | | | | | | | Atp2c1 | Elf4 |
| | | | | | | | | | | | Amot | Aifm1 |
| | | | | | | | | | | | 1600014k23rik | Rab33a |
| | | | | | | | | | | | Tbrg1 | Zfp280c |
| | | | | | | | | | | | Slit1 | Slc25a14 |
| | | | | | | | | | | | A730090h04rik | Gpr119 |
| | | | | | | | | | | | 4931406p16rik | Rbmx2 |
| | | | | | | | | | | | Opn3 | Gm595 |
| | | | | | | | | | | | Pdia4 | Enox2 |
| | | | | | | | | | | | B930054o08 | Gm14696 |
| | | | | | | | | | | | 1700031f05rik | Gm14697 |
| | | | | | | | | | | | Inhba | Arhgap36 |
| | | | | | | | | | | | Inhbb | Olfr1320 |
| | | | | | | | | | | | Helz | Olfr1321 |
| | | | | | | | | | | | Sele | Igsf1 |
| | | | | | | | | | | | Pdia6 | Olfr1322 |
| | | | | | | | | | | | Pdia5 | Olfr1323 |
| | | | | | | | | | | | Creb3 | Olfr1324 |
| | | | | | | | | | | | Efna1 | Stk26 |
| | | | | | | | | | | | Dlg5 | Frmd7 |
| | | | | | | | | | | | Procr | Rap2c |
| | | | | | | | | | | | Fgfr1 | Mbnl3 |
| | | | | | | | | | | | Gnb4 | Hs6st2 |
| | | | | | | | | | | | 2310030g06rik | Usp26 |
| | | | | | | | | | | | Gcm1 | 1700080016Rik |
| | | | | | | | | | | | Psg18 | Gpc4 |
| | | | | | | | | | | | Golt1b | Gpc3 |
| | | | | | | | | | | | Psg19 | Gm14582 |
| | | | | | | | | | | | Psg16 | A630012P03Rik |
| | | | | | | | | | | | Slc2a1 | Ccdc160 |
| | | | | | | | | | | | Psg17 | Phf6 |
| | | | | | | | | | | | Htra3 | Hprt |
| | | | | | | | | | | | Klhl13 | Gm28730 |
| | | | | | | | | | | | Ets2 | Plac1 |
| | | | | | | | | | | | Nppc | Fam122b |
| | | | | | | | | | | | Tgm1 | Fam122c |
| | | | | | | | | | | | Tmem108 | Mospd1 |
| | | | | | | | | | | | Usp53 | Etd |
| | | | | | | | | | | | Mark3 | Gm14597 |
| | | | | | | | | | | | Cbx8 | Cxx1c |
| | | | | | | | | | | | Hspa5 | Cxx1a |
| | | | | | | | | | | | Spats2 | Cxx1b |
| | | | | | | | | | | | Limk2 | 4930502E18Rik |
| | | | | | | | | | | | Mkl2 | 1700013H16Rik |
| | | | | | | | | | | | Shroom4 | Zfp36l3 |
| | | | | | | | | | | | Shroom1 | Xlr |
| | | | | | | | | | | | Pou2f3 | Gm16405 |
| | | | | | | | | | | | Acvr2b | Gm16430 |
| | | | | | | | | | | | Rbms2 | Slxl1 |
| | | | | | | | | | | | Atg4b | 3830403N18Rik |
| | | | | | | | | | | | Pappa2 | Gm773 |
| | | | | | | | | | | | Rbm25 | 1600025M17Rik |
| | | | | | | | | | | | Gm4793 | Zfp449 |
| | | | | | | | | | | | Nid1 | Gm2155 |
| | | | | | | | | | | | Uba6 | Smim10l2a |
| | | | | | | | | | | | Lamc1 | Gm2174 |
| | | | | | | | | | | | Slc40a1 | Ddx26b |
| | | | | | | | | | | | Hapln3 | Gm10477 |
| | | | | | | | | | | | Fam176a | Gm648 |
| | | | | | | | | | | | Pdlim1 | Mmgt1 |
| | | | | | | | | | | | Ube2q2 | Slc9a6 |
| | | | | | | | | | | | Au018091 | Fhl1 |
| | | | | | | | | | | | Bdkrb2 | Mtap7d3 |
| | | | | | | | | | | | E130203b14rik | Adgrg4 |
| | | | | | | | | | | | S100g | Brs3 |
| | | | | | | | | | | | 4933402el3rik | Htatsf1 |
| | | | | | | | | | | | Dapk2 | Vgll1 |
| | | | | | | | | | | | Gm11985 | Gm14718 |
| | | | | | | | | | | | Fndc3b | Cd40lg |
| | | | | | | | | | | | Twsg1 | Arhgef6 |
| | | | | | | | | | | | Aldh1a3 | Rbmx |
| | | | | | | | | | | | Lnx2 | Gm364 |
| | | | | | | | | | | | Taf7 | Gpr101 |
| | | | | | | | | | | | Ai844869 | Zic3 |
| | | | | | | | | | | | Clec12b | 4930550L24Rik |
| | | | | | | | | | | | Prkcsh | Fgfl3 |
| | | | | | | | | | | | Lama5 | F9 |
| | | | | | | | | | | | Tchh | Mcf2 |
| | | | | | | | | | | | Lama1 | Atp11c |
| | | | | | | | | | | | Rps6ka6 | Gm7073 |
| | | | | | | | | | | | Vhl | Gm14661 |
| | | | | | | | | | | | Eps8l2 | Sox3 |
| | | | | | | | | | | | Polg | Gm14662 |
| | | | | | | | | | | | | Gm14664 |
| | | | | | | | | | | | | Cdr1 |
| | | | | | | | | | | | | Ldoc1 |
| | | | | | | | | | | | | 4933402E13Rik |
| | | | | | | | | | | | | 4931400O07Rik |
| | | | | | | | | | | | | 1700019B21Rik |
| | | | | | | | | | | | | Gm6760 |
| | | | | | | | | | | | | 3830417A13Rik |
| | | | | | | | | | | | | Slitrk4 |
| | | | | | | | | | | | | Ctag2 |
| | | | | | | | | | | | | 4930447F04Rik |
| | | | | | | | | | | | | Slitrk2 |
| | | | | | | | | | | | | 1700036O09Rik |
| | | | | | | | | | | | | Gm1140 |
| | | | | | | | | | | | | Gm14692 |
| | | | | | | | | | | | | 4933436l01Rik |
| | | | | | | | | | | | | Fmr1os |
| | | | | | | | | | | | | Fmr1 |
| | | | | | | | | | | | | Fmr1nb |
| | | | | | | | | | | | | Gm14698 |
| | | | | | | | | | | | | Gm6812 |
| | | | | | | | | | | | | Gm14705 |
| | | | | | | | | | | | | Aff2 |
| | | | | | | | | | | | | 1700111N16Rik |
| | | | | | | | | | | | | 1700020N15Rik |
| | | | | | | | | | | | | Ids |
| | | | | | | | | | | | | 1110012L19Rik |
| | | | | | | | | | | | | 4930567H17Rik |
| | | | | | | | | | | | | BC023829 |
| | | | | | | | | | | | | Mamld1 |
| | | | | | | | | | | | | Mtm1 |
| | | | | | | | | | | | | Mtmr1 |
| | | | | | | | | | | | | Cd99l2 |
| | | | | | | | | | | | | Gm16189 |
| | | | | | | | | | | | | Hmgb3 |
| | | | | | | | | | | | | Gpr50 |
| | | | | | | | | | | | | Vma21 |
| | | | | | | | | | | | | Gm1141 |
| | | | | | | | | | | | | Prrg3 |
| | | | | | | | | | | | | Fate1 |
| | | | | | | | | | | | | Cnga2 |
| | | | | | | | | | | | | Magea4 |
| | | | | | | | | | | | | Gabre |
| | | | | | | | | | | | | Magea10 |
| | | | | | | | | | | | | Gabra3 |
| | | | | | | | | | | | | Gabrq |
| | | | | | | | | | | | | Cetn2 |
| | | | | | | | | | | | | Nsdhl |
| | | | | | | | | | | | | Gm14684 |
| | | | | | | | | | | | | Zfp185 |
| | | | | | | | | | | | | Pnma5 |
| | | | | | | | | | | | | Pnma3 |
| | | | | | | | | | | | | Xlr4a |
| | | | | | | | | | | | | Xlr3a |
| | | | | | | | | | | | | Xlr5a |
| | | | | | | | | | | | | Gm14685 |
| | | | | | | | | | | | | DXBay18 |
| | | | | | | | | | | | | Xlr5b |
| | | | | | | | | | | | | Spin2d |
| | | | | | | | | | | | | Xlr3b |
| | | | | | | | | | | | | Xlr4b |
| | | | | | | | | | | | | F8a |
| | | | | | | | | | | | | Xlr4c |
| | | | | | | | | | | | | Xlr3c |
| | | | | | | | | | | | | Xlr5c |
| | | | | | | | | | | | | RP23-95K12.13 |
| | | | | | | | | | | | | Zfp275 |
| | | | | | | | | | | | | Gm18336 |
| | | | | | | | | | | | | Gm26726 |
| | | | | | | | | | | | | Zfp92 |
| | | | | | | | | | | | | Trex2 |
| | | | | | | | | | | | | Haus7 |
| | | | | | | | | | | | | Bgn |
| | | | | | | | | | | | | Atp2b3 |
| | | | | | | | | | | | | Dusp9 |
| | | | | | | | | | | | | Pnck |
| | | | | | | | | | | | | Slc6a8 |
| | | | | | | | | | | | | Bcap31 |
| | | | | | | | | | | | | Abcd1 |
| | | | | | | | | | | | | Plxnb3 |
| | | | | | | | | | | | | Srpk3 |
| | | | | | | | | | | | | Idh3g |
| | | | | | | | | | | | | Ssr4 |
| | | | | | | | | | | | | Pdzd4 |
| | | | | | | | | | | | | L1cam |
| | | | | | | | | | | | | Arhgap4 |
| | | | | | | | | | | | | Avpr2 |
| | | | | | | | | | | | | Naa10 |
| | | | | | | | | | | | | Renbp |
| | | | | | | | | | | | | Hcfc1 |
| | | | | | | | | | | | | Irak1 |
| | | | | | | | | | | | | Mecp2 |
| | | | | | | | | | | | | Opn1mw |
| | | | | | | | | | | | | Tex28 |
| | | | | | | | | | | | | Tktl1 |
| | | | | | | | | | | | | Flna |
| | | | | | | | | | | | | Emd |
| | | | | | | | | | | | | RpI10 |
| | | | | | | | | | | | | Dnase1l1 |
| | | | | | | | | | | | | Taz |
| | | | | | | | | | | | | Atp6ap1 |
| | | | | | | | | | | | | Gdi1 |
| | | | | | | | | | | | | Fam50a |
| | | | | | | | | | | | | Plxna3 |
| | | | | | | | | | | | | Lage3 |
| | | | | | | | | | | | | Ubl4a |
| | | | | | | | | | | | | Slc10a3 |
| | | | | | | | | | | | | Fam3a |
| | | | | | | | | | | | | Ikbkg |
| | | | | | | | | | | | | G6pdx |
| | | | | | | | | | | | | Gm6880 |
| | | | | | | | | | | | | Olfr1326-ps1 |
| | | | | | | | | | | | | Olfr1325 |
| | | | | | | | | | | | | Gm5640 |
| | | | | | | | | | | | | Gm6890 |
| | | | | | | | | | | | | Gm5936 |
| | | | | | | | | | | | | Gab3 |
| | | | | | | | | | | | | Dkc1 |
| | | | | | | | | | | | | Mpp1 |
| | | | | | | | | | | | | Smim9 |
| | | | | | | | | | | | | F8 |
| | | | | | | | | | | | | Fundc2 |
| | | | | | | | | | | | | Cmc4 |
| | | | | | | | | | | | | Mtcp1 |
| | | | | | | | | | | | | Brcc3 |
| | | | | | | | | | | | | Vbp1 |
| | | | | | | | | | | | | Gm15384 |
| | | | | | | | | | | | | Rab39b |
| | | | | | | | | | | | | Gm15063 |
| | | | | | | | | | | | | Pls3 |
| | | | | | | | | | | | | Gm14715 |
| | | | | | | | | | | | | Gm14707 |
| | | | | | | | | | | | | Gm14717 |
| | | | | | | | | | | | | Cldn34b3 |
| | | | | | | | | | | | | Cldn34b4 |
| | | | | | | | | | | | | Cldn34d |
| | | | | | | | | | | | | Tbl1x |
| | | | | | | | | | | | | Prkx |
| | | | | | | | | | | | | Gm14742 |
| | | | | | | | | | | | | Pbsn |
| | | | | | | | | | | | | Gm14744 |
| | | | | | | | | | | | | 5430402E10Rik |
| | | | | | | | | | | | | Obp1a |
| | | | | | | | | | | | | Gm5938 |
| | | | | | | | | | | | | Obp1b |
| | | | | | | | | | | | | Gm14743 |
| | | | | | | | | | | | | 4930480E11Rik |
| | | | | | | | | | | | | Prrg1 |
| | | | | | | | | | | | | Fam47c |
| | | | | | | | | | | | | Gm7173 |
| | | | | | | | | | | | | Mageb16 |
| | | | | | | | | | | | | Gm26775 |
| | | | | | | | | | | | | Tmem47 |
| | | | | | | | | | | | | 4930595M18Rik |
| | | | | | | | | | | | | Dmd |
| | | | | | | | | | | | | Tsga8 |
| | | | | | | | | | | | | Fthl17a |
| | | | | | | | | | | | | Tab3Gk |
| | | | | | | | | | | | | Gm14764 |
| | | | | | | | | | | | | Gm14762 |
| | | | | | | | | | | | | 5430427O19Rik |
| | | | | | | | | | | | | Samt3 |
| | | | | | | | | | | | | Nr0b1 |
| | | | | | | | | | | | | Mageb4 |
| | | | | | | | | | | | | Il1rapl1 |
| | | | | | | | | | | | | Gm27000 |
| | | | | | | | | | | | | Pet2 |
| | | | | | | | | | | | | 4932429P05Rik |
| | | | | | | | | | | | | 4930415L06Rik |
| | | | | | | | | | | | | Gm44 |
| | | | | | | | | | | | | Gm14773 |
| | | | | | | | | | | | | Mageb2 |
| | | | | | | | | | | | | Gm5072 |
| | | | | | | | | | | | | Gm8914 |
| | | | | | | | | | | | | 1700084M14Rik |
| | | | | | | | | | | | | Gm14781 |
| | | | | | | | | | | | | Mageb5 |
| | | | | | | | | | | | | Mageb1 |
| | | | | | | | | | | | | Mageb18 |
| | | | | | | | | | | | | Gm5941 |
| | | | | | | | | | | | | 1700003E24Rik |
| | | | | | | | | | | | | BC061195 |
| | | | | | | | | | | | | Arx |
| | | | | | | | | | | | | Pola1 |
| | | | | | | | | | | | | Pcyt1b |
| | | | | | | | | | | | | Pdk3 |
| | | | | | | | | | | | | AU015836 |
| | | | | | | | | | | | | Gm14798 |
| | | | | | | | | | | | | Zfx |
| | | | | | | | | | | | | Eif2s3x |
| | | | | | | | | | | | | Klhl15 |
| | | | | | | | | | | | | Fam90a1b |
| | | | | | | | | | | | | Apoo |
| | | | | | | | | | | | | Gm14827 |
| | | | | | | | | | | | | Maged1 |
| | | | | | | | | | | | | Gspt2 |
| | | | | | | | | | | | | Zxdb |
| | | | | | | | | | | | | RP23-9K14.6 |
| | | | | | | | | | | | | Gm26617 |
| | | | | | | | | | | | | Spin4 |
| | | | | | | | | | | | | Arhgef9 |
| | | | | | | | | | | | | Amer1 |
| | | | | | | | | | | | | Asb12 |
| | | | | | | | | | | | | Zc4h2 |
| | | | | | | | | | | | | Zc3h12b |
| | | | | | | | | | | | | 1700010D01Rik |
| | | | | | | | | | | | | Las1l |
| | | | | | | | | | | | | Msn |
| | | | | | | | | | | | | F630028O10Rik |
| | | | | | | | | | | | | Vsig4 |
| | | | | | | | | | | | | Hsf3 |
| | | | | | | | | | | | | Heph |
| | | | | | | | | | | | | Gpr165 |
| | | | | | | | | | | | | Pgr15l |
| | | | | | | | | | | | | Eda2r |
| | | | | | | | | | | | | Ar |
| | | | | | | | | | | | | Ophn1 |
| | | | | | | | | | | | | Yipf6 |
| | | | | | | | | | | | | Stard8 |
| | | | | | | | | | | | | Efnb1 |
| | | | | | | | | | | | | Gm14812 |
| | | | | | | | | | | | | Gm14809 |
| | | | | | | | | | | | | Gm14808 |
| | | | | | | | | | | | | Pja1 |
| | | | | | | | | | | | | Tmem28 |
| | | | | | | | | | | | | Eda |
| | | | | | | | | | | | | Awat2 |
| | | | | | | | | | | | | Otud6a |
| | | | | | | | | | | | | Igbp1 |
| | | | | | | | | | | | | Dgat2l6 |
| | | | | | | | | | | | | Awat1 |
| | | | | | | | | | | | | P2ry4 |
| | | | | | | | | | | | | Arr3 |
| | | | | | | | | | | | | Pdzd11 |
| | | | | | | | | | | | | Kif4 |
| | | | | | | | | | | | | Gdpd2 |
| | | | | | | | | | | | | Gm14902 |
| | | | | | | | | | | | | Dlg3 |
| | | | | | | | | | | | | Tex11 |
| | | | | | | | | | | | | Slc7a3 |
| | | | | | | | | | | | | Snx12 |
| | | | | | | | | | | | | Foxo4 |
| | | | | | | | | | | | | Gm614 |
| | | | | | | | | | | | | Gm20489 |
| | | | | | | | | | | | | Il2rg |
| | | | | | | | | | | | | Medl2 |
| | | | | | | | | | | | | Nlgn3 |
| | | | | | | | | | | | | Gjb1 |
| | | | | | | | | | | | | Zmym3 |
| | | | | | | | | | | | | Nono |
| | | | | | | | | | | | | Itgb1bp2 |
| | | | | | | | | | | | | Taf1 |
| | | | | | | | | | | | | Ogt |
| | | | | | | | | | | | | Cxcr3 |
| | | | | | | | | | | | | Gm4779 |
| | | | | | | | | | | | | 8030474K03Rik |
| | | | | | | | | | | | | Nhsl2 |
| | | | | | | | | | | | | Rgag4 |
| | | | | | | | | | | | | Pin4 |
| | | | | | | | | | | | | Ercc6l |
| | | | | | | | | | | | | Rps4x |
| | | | | | | | | | | | | Cited1 |
| | | | | | | | | | | | | Hdac8 |
| | | | | | | | | | | | | Phka1 |
| | | | | | | | | | | | | Gm9112 |
| | | | | | | | | | | | | Dmrtc1b |
| | | | | | | | | | | | | Dmrtc1c1 |
| | | | | | | | | | | | | Dmrtc1c2 |
| | | | | | | | | | | | | 1700031F05Rik |
| | | | | | | | | | | | | Dmrtc1a |
| | | | | | | | | | | | | 1700011M02Rik |
| | | | | | | | | | | | | Nap1l2 |
| | | | | | | | | | | | | Cdx4 |
| | | | | | | | | | | | | Chic1 |
| | | | | | | | | | | | | Gm26952 |
| | | | | | | | | | | | | Tsx |
| | | | | | | | | | | | | Gm26992 |
| | | | | | | | | | | | | Tsix |
| | | | | | | | | | | | | Xist |
| | | | | | | | | | | | | Jpx |
| | | | | | | | | | | | | Ftx |
| | | | | | | | | | | | | Zcchc13 |
| | | | | | | | | | | | | Slc16a2 |
| | | | | | | | | | | | | Rlim |
| | | | | | | | | | | | | C77370 |
| | | | | | | | | | | | | Abcb7 |
| | | | | | | | | | | | | Uprt |
| | | | | | | | | | | | | Zdhhc15 |
| | | | | | | | | | | | | 1700121L16Rik |
| | | | | | | | | | | | | Magee2 |
| | | | | | | | | | | | | Pbdc1 |
| | | | | | | | | | | | | Magee1 |
| | | | | | | | | | | | | 5330434G04Rik |
| | | | | | | | | | | | | Cypt2 |
| | | | | | | | | | | | | Fgf16 |
| | | | | | | | | | | | | Atrx |
| | | | | | | | | | | | | Magt1 |
| | | | | | | | | | | | | Cox7b |
| | | | | | | | | | | | | Atp7a |
| | | | | | | | | | | | | Tlr13 |
| | | | | | | | | | | | | Pgk1 |
| | | | | | | | | | | | | Taf9b |
| | | | | | | | | | | | | Fnd3c2 |
| | | | | | | | | | | | | Fndc3c1 |
| | | | | | | | | | | | | Cysltr1 |
| | | | | | | | | | | | | Gm5127 |
| | | | | | | | | | | | | Zcchc5 |
| | | | | | | | | | | | | Lpar4 |
| | | | | | | | | | | | | P2ry10 |
| | | | | | | | | | | | | A630033H20Rik |
| | | | | | | | | | | | | Gpr174 |
| | | | | | | | | | | | | Itm2a |
| | | | | | | | | | | | | Tbx22 |
| | | | | | | | | | | | | 2610002M06Rik |
| | | | | | | | | | | | | Fam46d |
| | | | | | | | | | | | | Gm732 |
| | | | | | | | | | | | | Gm379 |
| | | | | | | | | | | | | Brwd3 |
| | | | | | | | | | | | | Hmgn5 |
| | | | | | | | | | | | | Sh3bgr1 |
| | | | | | | | | | | | | Gm6377 |
| | | | | | | | | | | | | RP23-240M8.2 |
| | | | | | | | | | | | | Pou3f4 |
| | | | | | | | | | | | | Cylc1 |
| | | | | | | | | | | | | Gm10112 |
| | | | | | | | | | | | | Rps6ka6 |
| | | | | | | | | | | | | Hdx |
| | | | | | | | | | | | | RP23-466J17.3 |
| | | | | | | | | | | | | Tex16 |
| | | | | | | | | | | | | 4933403O08Rik |
| | | | | | | | | | | | | Apool |
| | | | | | | | | | | | | Satl1 |
| | | | | | | | | | | | | 2010106E10Rik |
| | | | | | | | | | | | | Zfp711 |
| | | | | | | | | | | | | Pof1b |
| | | | | | | | | | | | | Gm14936 |
| | | | | | | | | | | | | Chm |
| | | | | | | | | | | | | Dach2 |
| | | | | | | | | | | | | Klhl4 |
| | | | | | | | | | | | | Ube2dnl1 |
| | | | | | | | | | | | | Ube2dnl2 |
| | | | | | | | | | | | | 4930555B12Rik |
| | | | | | | | | | | | | Cpxcr1 |
| | | | | | | | | | | | | H2afb2 |
| | | | | | | | | | | | | Gm14920 |
| | | | | | | | | | | | | Gm28579 |
| | | | | | | | | | | | | Tgif2lx2 |
| | | | | | | | | | | | | Tgif2lx1 |
| | | | | | | | | | | | | Gm14929 |
| | | | | | | | | | | | | Pabpc5 |
| | | | | | | | | | | | | Pcdh11x |
| | | | | | | | | | | | | H2afb3 |
| | | | | | | | | | | | | Nap1l3 |
| | | | | | | | | | | | | Gm17521 |
| | | | | | | | | | | | | Cldn34c1 |
| | | | | | | | | | | | | Astx6 |
| | | | | | | | | | | | | Srsx |
| | | | | | | | | | | | | Gm17577 |
| | | | | | | | | | | | | Gm14951 |
| | | | | | | | | | | | | Astx2 |
| | | | | | | | | | | | | Gm17412 |
| | | | | | | | | | | | | Cldn34c2 |
| | | | | | | | | | | | | Gm14950 |
| | | | | | | | | | | | | Gm17467 |
| | | | | | | | | | | | | Cldn34c3 |
| | | | | | | | | | | | | Astx5 |
| | | | | | | | | | | | | Vmn2r121 |
| | | | | | | | | | | | | Astx1a |
| | | | | | | | | | | | | Gm17584 |
| | | | | | | | | | | | | Astx4a |
| | | | | | | | | | | | | Gm17469 |
| | | | | | | | | | | | | Astx4b |
| | | | | | | | | | | | | Astx1b |
| | | | | | | | | | | | | Gm17361 |
| | | | | | | | | | | | | Gm21616 |
| | | | | | | | | | | | | Astx4c |
| | | | | | | | | | | | | Gm17693 |
| | | | | | | | | | | | | Astx1c |
| | | | | | | | | | | | | Gm17522 |
| | | | | | | | | | | | | Astx4d |
| | | | | | | | | | | | | Gm17267 |
| | | | | | | | | | | | | Astx3 |
| | | | | | | | | | | | | 4932411N23Rik |
| | | | | | | | | | | | | Gm382 |
| | | | | | | | | | | | | 4921511C20Rik |
| | | | | | | | | | | | | Cldn34c4 |
| | | | | | | | | | | | | 4930558G05Rik |
| | | | | | | | | | | | | Diaph2 |
| | | | | | | | | | | | | Pcdh19 |
| | | | | | | | | | | | | Gm26851 |
| | | | | | | | | | | | | Tnmd |
| | | | | | | | | | | | | Tspan6 |
| | | | | | | | | | | | | Srpx2 |
| | | | | | | | | | | | | Sytl4 |
| | | | | | | | | | | | | Cstf2 |
| | | | | | | | | | | | | Nox1 |
| | | | | | | | | | | | | Xkrx |
| | | | | | | | | | | | | Arl13a |
| | | | | | | | | | | | | Trmt2b |
| | | | | | | | | | | | | Tmem35 |
| | | | | | | | | | | | | Cenpi |
| | | | | | | | | | | | | Drp2 |
| | | | | | | | | | | | | Taf7l |
| | | | | | | | | | | | | Timm8a1 |
| | | | | | | | | | | | | Btk |
| | | | | | | | | | | | | Rpl36a |
| | | | | | | | | | | | | Gla |
| | | | | | | | | | | | | Hnrnph2 |
| | | | | | | | | | | | | Armcx4 |
| | | | | | | | | | | | | Armcx1 |
| | | | | | | | | | | | | Armcx6 |
| | | | | | | | | | | | | Armcx3 |
| | | | | | | | | | | | | Armcx2 |
| | | | | | | | | | | | | Nxf2 |
| | | | | | | | | | | | | Zmat1 |
| | | | | | | | | | | | | Gm15023 |
| | | | | | | | | | | | | Tceal6 |
| | | | | | | | | | | | | Pramel3 |
| | | | | | | | | | | | | Gm5128 |
| | | | | | | | | | | | | Gm7903 |
| | | | | | | | | | | | | AV320801 |
| | | | | | | | | | | | | Nxf7 |
| | | | | | | | | | | | | Prame |
| | | | | | | | | | | | | Tcp11x2 |
| | | | | | | | | | | | | Tmsb15a |
| | | | | | | | | | | | | Armcx5 |
| | | | | | | | | | | | | Gprasp1 |
| | | | | | | | | | | | | Bhlhb9 |
| | | | | | | | | | | | | Gprasp2 |
| | | | | | | | | | | | | Arxes2 |
| | | | | | | | | | | | | Arxes1 |
| | | | | | | | | | | | | Bex2 |
| | | | | | | | | | | | | Nxf3 |
| | | | | | | | | | | | | Bex4 |
| | | | | | | | | | | | | Tceal8 |
| | | | | | | | | | | | | Tceal5 |
| | | | | | | | | | | | | Bex1 |
| | | | | | | | | | | | | Tceal7 |
| | | | | | | | | | | | | Wbp5 |
| | | | | | | | | | | | | Ngfrap1 |
| | | | | | | | | | | | | Kir3dl2 |
| | | | | | | | | | | | | Kir3dl1 |
| | | | | | | | | | | | | Tceal3 |
| | | | | | | | | | | | | Tceal1 |
| | | | | | | | | | | | | Morf4l2 |
| | | | | | | | | | | | | Glra4 |
| | | | | | | | | | | | | Plp1 |
| | | | | | | | | | | | | Rab9b |
| | | | | | | | | | | | | H2bfm |
| | | | | | | | | | | | | Tmsb15l |
| | | | | | | | | | | | | Tmsb15b2 |
| | | | | | | | | | | | | Tmsb15b1 |
| | | | | | | | | | | | | Slc25a53 |
| | | | | | | | | | | | | Zcchc18 |
| | | | | | | | | | | | | Fam199x |
| | | | | | | | | | | | | Esx1 |
| | | | | | | | | | | | | Il1rap12 |
| | | | | | | | | | | | | Tex13a |
| | | | | | | | | | | | | Nrk |
| | | | | | | | | | | | | Serpina7 |
| | | | | | | | | | | | | 4930513O06Rik |
| | | | | | | | | | | | | 4933428M09Rik |
| | | | | | | | | | | | | Mum1l1 |
| | | | | | | | | | | | | Trap1a |
| | | | | | | | | | | | | D330045A20Rik |
| | | | | | | | | | | | | Rnf128 |
| | | | | | | | | | | | | Tbc1d8b |
| | | | | | | | | | | | | Gm15013 |
| | | | | | | | | | | | | Ripply1 |
| | | | | | | | | | | | | Cldn2 |
| | | | | | | | | | | | | Morc4 |
| | | | | | | | | | | | | Rbm41 |
| | | | | | | | | | | | | Nup62cl |
| | | | | | | | | | | | | Pih1h3b |
| | | | | | | | | | | | | Gm15046 |
| | | | | | | | | | | | | Frmpd3 |
| | | | | | | | | | | | | Prps1 |
| | | | | | | | | | | | | Tsc22d3 |
| | | | | | | | | | | | | Mid2 |
| | | | | | | | | | | | | Eif2c5 |
| | | | | | | | | | | | | Tex13 |
| | | | | | | | | | | | | Vsig1 |
| | | | | | | | | | | | | Psmd10 |
| | | | | | | | | | | | | Atg4a |
| | | | | | | | | | | | | Col4a6 |
| | | | | | | | | | | | | Col4a5 |
| | | | | | | | | | | | | Irs4 |
| | | | | | | | | | | | | Gm15295 |
| | | | | | | | | | | | | Gm15294 |
| | | | | | | | | | | | | Gm15298 |
| | | | | | | | | | | | | Gucy2f |
| | | | | | | | | | | | | Nxt2 |
| | | | | | | | | | | | | Kcne1l |
| | | | | | | | | | | | | Acsl4 |
| | | | | | | | | | | | | Tmem164 |
| | | | | | | | | | | | | Ammecr1 |
| | | | | | | | | | | | | Rgag1 |
| | | | | | | | | | | | | Chrdl1 |
| | | | | | | | | | | | | Pak3 |
| | | | | | | | | | | | | Capn6 |
| | | | | | | | | | | | | Dcx |
| | | | | | | | | | | | | A730046J19Rik |
| | | | | | | | | | | | | Alg13 |
| | | | | | | | | | | | | Trpc5 |
| | | | | | | | | | | | | Trpc5os |
| | | | | | | | | | | | | Zcchc16 |
| | | | | | | | | | | | | Lhfpl1 |
| | | | | | | | | | | | | Amot |
| | | | | | | | | | | | | Htr2c |
| | | | | | | | | | | | | Il13ra2 |
| | | | | | | | | | | | | Lrch2 |
| | | | | | | | | | | | | Gm15128 |
| | | | | | | | | | | | | Gm15080 |
| | | | | | | | | | | | | Gm15107 |
| | | | | | | | | | | | | Gm15114 |
| | | | | | | | | | | | | Gm8334 |
| | | | | | | | | | | | | Gm15127 |
| | | | | | | | | | | | | Luzp4 |
| | | | | | | | | | | | | Gm15099 |
| | | | | | | | | | | | | Ott |
| | | | | | | | | | | | | Gm15092 |
| | | | | | | | | | | | | Gm15093 |
| | | | | | | | | | | | | Gm5100 |
| | | | | | | | | | | | | Gm15085 |
| | | | | | | | | | | | | Gm15086 |
| | | | | | | | | | | | | Gm10439 |
| | | | | | | | | | | | | Gm15097 |
| | | | | | | | | | | | | Gm15091 |
| | | | | | | | | | | | | Gm15104 |
| | | | | | | | | | | | | Tmem29 |
| | | | | | | | | | | | | Apex2 |
| | | | | | | | | | | | | Alas2 |
| | | | | | | | | | | | | Pfkfb1 |
| | | | | | | | | | | | | Tro |
| | | | | | | | | | | | | Maged2 |
| | | | | | | | | | | | | Gm27191 |
| | | | | | | | | | | | | Gnl3l |
| | | | | | | | | | | | | Fgd1 |
| | | | | | | | | | | | | Tsr2 |
| | | | | | | | | | | | | Gm15138 |
| | | | | | | | | | | | | Wnk3 |
| | | | | | | | | | | | | A230072E10Rik |
| | | | | | | | | | | | | Fam120c |
| | | | | | | | | | | | | Phf8 |
| | | | | | | | | | | | | Huwe1 |
| | | | | | | | | | | | | Hsd17b10 |
| | | | | | | | | | | | | Ribc1 |
| | | | | | | | | | | | | Smc1a |
| | | | | | | | | | | | | Iqsec2 |
| | | | | | | | | | | | | Kdm5c |
| | | | | | | | | | | | | Kantr |
| | | | | | | | | | | | | Tspyl2 |
| | | | | | | | | | | | | Gpr173 |
| | | | | | | | | | | | | Cldn34a |
| | | | | | | | | | | | | Shroom2 |
| | | | | | | | | | | | | Gpr143 |
| | | | | | | | | | | | | Usp51 |
| | | | | | | | | | | | | Mageh1 |
| | | | | | | | | | | | | Foxr2 |
| | | | | | | | | | | | | Rragb |
| | | | | | | | | | | | | Klf8 |
| | | | | | | | | | | | | Ubqln2 |
| | | | | | | | | | | | | Cypt3 |
| | | | | | | | | | | | | Kctd12b |
| | | | | | | | | | | | | RP23-106P7.5 |
| | | | | | | | | | | | | 2210013O21Rik |
| | | | | | | | | | | | | Spin2c |
| | | | | | | | | | | | | Samt1 |
| | | | | | | | | | | | | 4921511M17Rik |
| | | | | | | | | | | | | Gm10057 |
| | | | | | | | | | | | | Gm15140 |
| | | | | | | | | | | | | 4930524N10Rik |
| | | | | | | | | | | | | Samt4 |
| | | | | | | | | | | | | Samt2 |
| | | | | | | | | | | | | Cldn34b1 |
| | | | | | | | | | | | | Magea6 |
| | | | | | | | | | | | | Magea3 |
| | | | | | | | | | | | | Magea8 |
| | | | | | | | | | | | | Magea2 |
| | | | | | | | | | | | | Magea5 |
| | | | | | | | | | | | | Magea1 |
| | | | | | | | | | | | | Cldn34b2 |
| | | | | | | | | | | | | Sat1 |
| | | | | | | | | | | | | Acot9 |
| | | | | | | | | | | | | Prdx4 |
| | | | | | | | | | | | | Ptchd1 |
| | | | | | | | | | | | | Gm15156 |
| | | | | | | | | | | | | Gm15155 |
| | | | | | | | | | | | | Phex |
| | | | | | | | | | | | | Sms |
| | | | | | | | | | | | | Mbtps2 |
| | | | | | | | | | | | | Yy2 |
| | | | | | | | | | | | | Smpx |
| | | | | | | | | | | | | Gm15169 |
| | | | | | | | | | | | | Klhl34 |
| | | | | | | | | | | | | Cnksr2 |
| | | | | | | | | | | | | Rps6ka3 |
| | | | | | | | | | | | | Eif1ax |
| | | | | | | | | | | | | Map7d2 |
| | | | | | | | | | | | | A830080D01Rik |
| | | | | | | | | | | | | Sh3kbp1 |
| | | | | | | | | | | | | Map3k15 |
| | | | | | | | | | | | | Pdha1 |
| | | | | | | | | | | | | Adgrg2 |
| | | | | | | | | | | | | Gm15241 |
| | | | | | | | | | | | | Phka2 |
| | | | | | | | | | | | | Gm15243 |
| | | | | | | | | | | | | Ppef1 |
| | | | | | | | | | | | | Rs1 |
| | | | | | | | | | | | | Cdkl5 |
| | | | | | | | | | | | | Gja6 |
| | | | | | | | | | | | | Scml2 |
| | | | | | | | | | | | | Gm15262 |
| | | | | | | | | | | | | Rai2 |
| | | | | | | | | | | | | Scml1 |
| | | | | | | | | | | | | Gm15205 |
| | | | | | | | | | | | | Nhs |
| | | | | | | | | | | | | Gm15202 |
| | | | | | | | | | | | | Reps2 |
| | | | | | | | | | | | | Rbbp7 |
| | | | | | | | | | | | | Txlng |
| | | | | | | | | | | | | Syap1 |
| | | | | | | | | | | | | Ctps2 |
| | | | | | | | | | | | | S100g |
| | | | | | | | | | | | | Grpr |
| | | | | | | | | | | | | Rnf138rt1 |
| | | | | | | | | | | | | Ap1s2 |
| | | | | | | | | | | | | Zrsr2 |
| | | | | | | | | | | | | Car5b |
| | | | | | | | | | | | | Siah1b |
| | | | | | | | | | | | | Tmem27 |
| | | | | | | | | | | | | Ace2 |
| | | | | | | | | | | | | Bmx |
| | | | | | | | | | | | | Pir |
| | | | | | | | | | | | | Figf |
| | | | | | | | | | | | | Piga |
| | | | | | | | | | | | | Asb11 |
| | | | | | | | | | | | | Asb9 |
| | | | | | | | | | | | | Mospd2 |
| | | | | | | | | | | | | Fancb |
| | | | | | | | | | | | | Gm17604 |
| | | | | | | | | | | | | Glra2 |
| | | | | | | | | | | | | Gemin8 |
| | | | | | | | | | | | | Gpm6b |
| | | | | | | | | | | | | Ofd1 |
| | | | | | | | | | | | | Trappc2 |
| | | | | | | | | | | | | Rab9 |
| | | | | | | | | | | | | Tceanc |
| | | | | | | | | | | | | Egfl6 |
| | | | | | | | | | | | | Gm15226 |
| | | | | | | | | | | | | Gm1720 |
| | | | | | | | | | | | | Gm15230 |
| | | | | | | | | | | | | Gm8817 |
| | | | | | | | | | | | | Gm15232 |
| | | | | | | | | | | | | Gm15228 |
| | | | | | | | | | | | | Tmsb4X |
| | | | | | | | | | | | | Tlr8 |
| | | | | | | | | | | | | Tlr7 |
| | | | | | | | | | | | | Prps2 |
| | | | | | | | | | | | | Gm15239 |
| | | | | | | | | | | | | Frmpd4 |
| | | | | | | | | | | | | Msl3 |
| | | | | | | | | | | | | Arhgap6 |
| | | | | | | | | | | | | Gm15261 |
| | | | | | | | | | | | | Amelx |
| | | | | | | | | | | | | Hccs |
| | | | | | | | | | | | | Gm15245 |
| | | | | | | | | | | | | Mid1 |
| | | | | | | | | | | | | 4933400A11Rik |
| | | | | | | | | | | | | Gm15726 |
| | | | | | | | | | | | | Gm15247 |
| | | | | | | | | | | | | Gm21887 |
| | | | | | | | | | | | | Asmt |

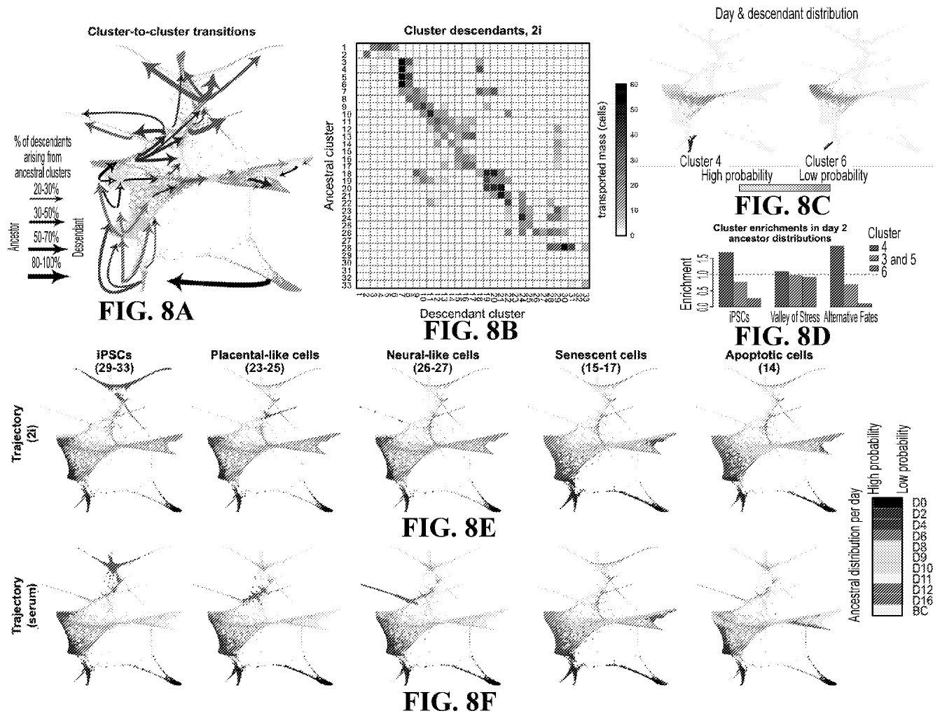

As an additional validation, we modified an existing trajectory finding technique, Wishbone(S10)—based on shortest paths in k-NN graphs—to include information about time and proliferation. This gives trajectories whose overall shape agrees with the transports displayed in FIG. 8A.

### Learning Gene Regulatory Networks

How to set up an optimization problem to solve for a regulatory function that fits the transport maps is described above.

In order to make this concrete, a function class F was specified over which to optimize. Consider a rectified-linear function class defined in terms of a specific generalized logistic function

$$𝓁 ( x ; k , b , {y}_{0} , {x}_{0} ) = \frac{k {y}_{0}}{{y}_{0} + ( k - {y}_{0} ) {e}^{- b ( x - {x}_{0} )}} ,$$

where k, b, y0, x0 ∈R are parameters of the generalized logistic function 1(x). A function class F is defined consisting of functions f: RG→RG of the form

ƒ(x)=U [symbol] (WTx),

where 1 is applied entry-wise to the vector WZx∈R^{M} to obtain a vector that we multiply against U∈RG×M. Here T∈RGTF×G denotes a projection operator that selects only the coordinates of x that are transcription factors, and GTF is the number of transcription factors.

The following optimization over matrices U∈RG×M and W∈RM×GTF

$${min}_{U , W} {𝔼}_{r} {‖ \frac{{X}_{{t}_{i}} - {X}_{{t}_{i + 1}}}{{Δ}_{t}} - U 𝓁 ( {WTX}_{{t}_{i}} ) ‖}^{2} + {η}_{1} {‖ U ‖}_{1} + {η}_{2} {‖ W ‖}_{1} , \\ + {η}_{3} {‖ W ‖}_{2}^{2}$$

$$s . t . U \geq 0.$$

where (Xₜᵢ, Xₜᵢ₊₁) is a pair of random variables distributed according to the normalized transport map r and //U//₁ denotes the sparsity-promoting l₁ norm of U, viewed as a vector (that is, the sum of the absolute value of the entries of U). Each rank one component (row of U or column of W) gives us a group of genes controlled by a set of transcription factors. The regularization parameters η₁ and η₂ control the sparsity level (i.e. number of genes in these groups).

Implementation:

A stochastic gradient descent algorithm was designed to solve [10]. Over a sequence of epochs, the algorithm samples batches of points (Xₜᵢ, Xₜᵢ₊₁) from the transport maps, computes the gradient of the loss, and updates the optimization variables U and W. The batch sizes are determined by the Shannon diversity of the transport maps: for each pair of consecutive time points, the Shannon diversity S was computed of the transport map, then randomly sample max(S×10−5, 10) pairs of points to add to the batch. We run for a total of 10,000 epochs.

This algorithm was implemented in Python.

### 7. Clustering Cells

Cells were clustered using the Louvain-Jaccard community detection algorithm (S19-S21) in 20 dimensional diffusion component space. This algorithm maximizes the Louvain modularity—a value between −1 and 1 that measures the density of links inside communities compared to links between communities.

As a first step, the 20-nearest neighbor graph in 20 dimensional diffusion component space (computed on cells from both 2i and serum) were computed. The edges are weighted in this graph by the Jaccard similarity coefficient. The resulting graph was partitioned into clusters using the Louvain community detection algorithm (S19) implemented in the function multilevel. community from the R pack-age IGRAPH (1.0.1) (S22). The default parameters for automatically selecting the number of clusters gave us 33 clusters, displayed in FIG. 7D.

### 8. Gene Correlation Modules Reveal Biological Signatures

In this section technique for identifying modules of correlated genes are described, with the goal of revealing coherent biological processes.

The procedure consists of two steps. In the first step, the Graphical Lasso (S23) was used to compute a regularized estimate of the covariance matrix for the 66,000 expression profiles. The Graphical Lasso fits a covariance matrix to the data, regularized so that the inverse of the covariance matrix is sparse (i.e. has only a few non-zeros). The motivation for selecting a sparse inverse covariance is based on the fact that if a collection of observations have a multivariate Gaussian distribution with mean t and covariance X, then the zero pattern of E-1 completely specifies the conditional independence structure of the observations:
Σᵢⱼ⁻¹=0⇔variables i and j are conditionally independent given the other variables. Let Θ=Σ⁻¹ and let S denote the empirical covariance for our expression profiles

The Graphical Lasso maximizes the Gaussian log likelihood:

$${maximize}_{Θ} log det Θ - tr ( S Θ ) - ρ {‖ Θ ‖}_{1} .$$

Here ∥Θ∥₁ is a regularization term that promotes sparse solutions. The optimal Θ is a (regularized) maximum-likelihood estimate of the inverse covariance matrix E-1 for a Gaussian ensemble.

Gene modules were identifed as tightly knit communities in the network specified by Θ (see below). Based on these gene modules, we then identified gene signatures related to specific pathways, cell types, and conditions. We did this by functional enrichment analysis (see below). The gene modules are displayed in FIG. 13.

Computing gene modules: The glasso package was used (S23) to solve the graphical lasso optimization problem. The regularization parameter ρ was tuned to achieve a desirable sparsity level for Θ. In particular, we select a value of ρ that gave around 10,000 total genes (i.e. 10,000 non-zero rows and columns of Θ).

Viewing Θ as an adjacency matrix defining a network of genes, we partitioned the network using with the Infomap community detection algorithm (S24) from the R package IGRAPH (v1.1.0) (S22), retaining modules that contain more than 10 genes. This yields 44 gene modules, each consisting of a set of genes. The modules are visualized in FIG. 13.

Functional Enrichments:

Functional enrichment analysis was performed on the gene sets defined by the modules using the findGO.pl program from the HOMER suite (Hypergeometric Optimization of Motif Enrichment, version: 4.9.1) (S12) with Benjamini and Hochberg correction for multiple hypothesis testing (retaining terms at adjusted p-value<0.05). All genes that passed quality-control filters were used as a background set.

This yielded a set of biological signatures related to each module.

Computing scores from gene sets Given a set of genes (coming from a gene module or biological signature), cells were scored based on their gene expression. In particular, for a given cell the z-score for each gene in the set was determined. The z-scores were then truncated at 5 or −5, and define the signature of the cell to be the mean z-score over all genes in the gene set. The scores for the gene modules are visualized in FIG. 13 and the scores for the biological signatures are visualized in FIGS. 7A-7F.

### Example 2 Reprogramming to iPSCs as a Test Case for Analysis of Developmental Landscapes

WADDINGTON-OT was used to analyze the reprogramming of fibroblasts to iPSCs (39-42).

Studies have applied scRNA-Seq, but they have involved only several dozen cells or several dozen genes (13, 43). Studies have proposed that reprogramming involves two “transcriptional waves,” with gain of proliferation and loss of fibroblast identity followed by transient activation of developmental regulators and gradual activation of embryonic stem cell (ESC) genes (12). Some studies (16, 44, 45), have noted strong upregulation of lineage-specific genes from unrelated lineages (e.g., related to neurons), but it has been unclear whether this largely reflects disorganized gene activation by TFs or coherent differentiation of specific (off-target) cell types (45).

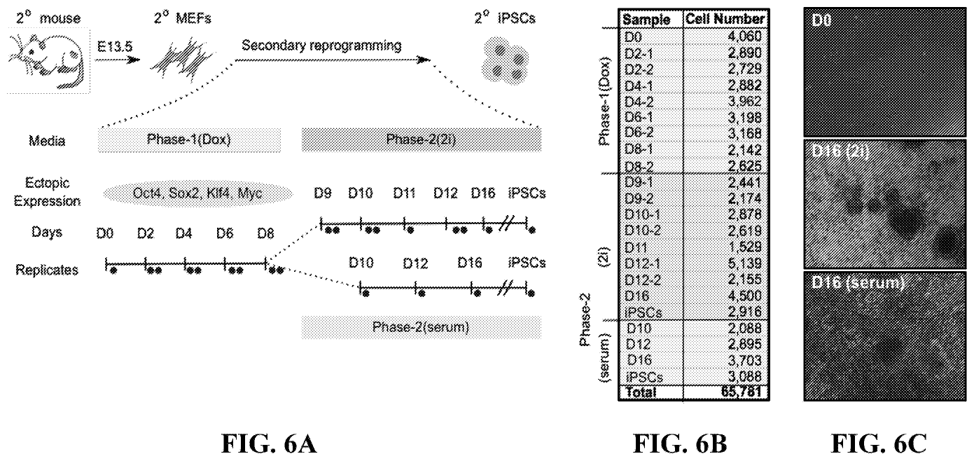

scRNA-seq profiles of 65,781 cells were collected across a 16-day time course of iPSC induction, under two conditions (FIGS. 6A,6B). An efficient “secondary” reprogramming system was used (46), as described hereinbelow.

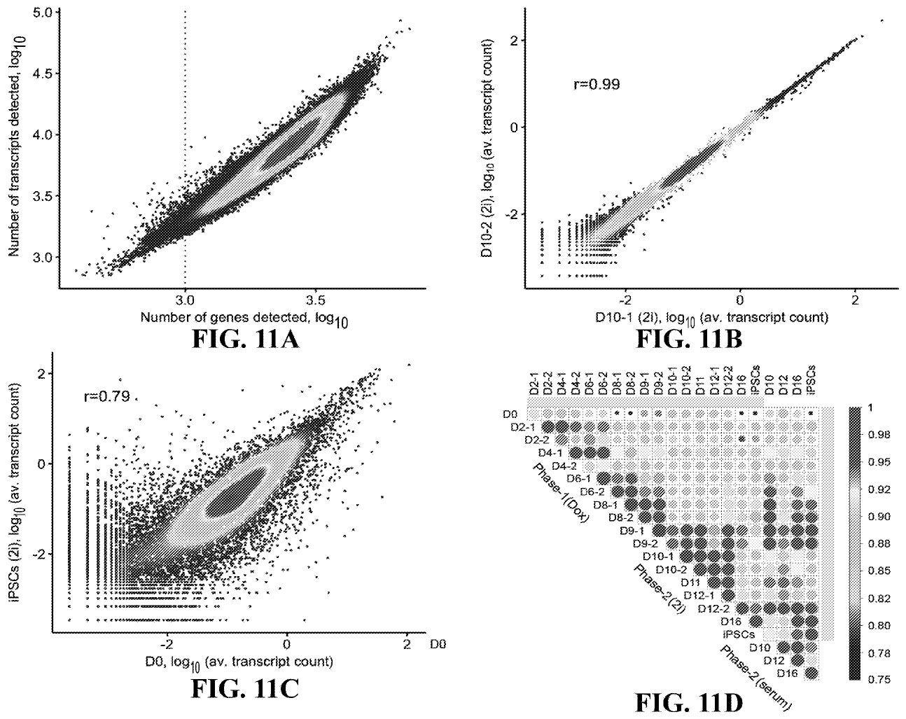

Mouse embryonic fibroblasts (MEFs) were obtained from a single female embryo homozygous for ROSA26-M2rtTA, which constitutively expresses a reverse transactivator controlled by doxycycline (Dox), a Dox-inducible polycistronic cassette carrying Pou5f1 (Oct4), Klf4, Sox2, and Myc (OKSM), and an EGFP reporter incorporated into the endogenous Oct4 locus (Oct4-IRES-EGFP). MEFs were plated in serum-containing induction medium, with Dox added on day 0 to induce the OKSM cassette (Phase-1(Dox)). Following Dox withdrawal at day 8, cells were transferred to either serum-free N2B27 2i medium (Phase-2(2i)) or maintained in serum (Phase-2(serum)). Oct4 EGFP+ cells emerged on day 10 as a reporter for “successful” reprogramming to endogenous Oct4 expression (FIG. 6C). Single or duplicate samples were collected at the various time points (FIG. 6A), single cell suspensions were generated and scRNA-Seq (Table 8, FIGS. 11A-11D) was performed. Samples were also collected from established iPSC lines reprogrammed from the same MEFs, maintained in either 2i or serum conditions. Overall, 68,339 cells were programmed to an average depth of 38,462 reads per cell (Table 8). After discarding cells with less than 1,000 genes detected, a total of 65,781 cells were retained, with a median of 2,398 genes and 7,387 unique transcripts per cell.

TABLE 8

| | | | | | Mean | | Median | |
|---|---|---|---|---|---|---|---|---|
| | | Number | Number | | Reads | Median | UMI | cDNA PCR |
| Sample | | of | of cells | Number of | per | Genes | Counts per | Duplication |
| (Day) | Phase | Cells | (filtered) | reads | Cells | per Cell | Cell | % |
| D 0 | Dox | 4241 | 4060 | 111,286,101 | 26240 | 2446 | 6495 | 50.5 |
| D 2-1 | Dox | 2909 | 2890 | 143,713,479 | 49403 | 2867 | 8401 | 55.6 |
| D 2-2 | Dox | 2758 | 2729 | 109,907,870 | 39850 | 2521 | 6271 | 70.2 |
| D 4-1 | Dox | 2889 | 2882 | 126,824,856 | 43899 | 2447 | 7349 | 57.3 |
| D 4-2 | Dox | 3976 | 3962 | 99,109,221 | 24926 | 2386 | 7446 | 34.1 |
| D 6-1 | Dox | 3676 | 3198 | 132,565,146 | 36062 | 1453 | 3147 | 84 |
| D 6-2 | Dox | 3534 | 3168 | 99,748,307 | 28225 | 1533 | 3567 | 76.5 |
| D 8-1 | Dox | 2177 | 2142 | 98,462,446 | 45228 | 2332 | 8216 | 65.7 |
| D 8-2 | Dox | 3677 | 2625 | 95,807,550 | 26055 | 1486 | 3862 | 62.6 |
| D 9-1 | 2i | 2445 | 2441 | 122,451,561 | 50082 | 2843 | 11799 | 51.8 |
| D 9-2 | 2i | 2183 | 2174 | 125,014,976 | 57267 | 2734 | 11183 | 57 |
| D 10-1 | 2i | 2878 | 2878 | 129,837,247 | 45113 | 2625 | 9570 | 58.1 |
| D 10-2 | 2i | 2620 | 2619 | 126,364,110 | 48230 | 2647 | 9930 | 59.5 |
| D 11 | 21 | 1532 | 1529 | 119,736,956 | 78157 | 2892 | 10744 | 65.9 |
| D 12-1 | 2i | 5144 | 5139 | 158,679,538 | 30847 | 2269 | 6299 | 41 |
| D 12-2 | 2i | 2156 | 2155 | 112,512,277 | 52185 | 2651 | 8633 | 54.8 |
| D 16 | 2i | 4621 | 4500 | 117,242,910 | 25371 | 2203 | 7761 | 39.5 |
| iPSCs | 2i | 2917 | 2916 | 139,441,360 | 47803 | 3172 | 12775 | 38.2 |
| D 10 | serum | 2094 | 2088 | 115,832,953 | 55316 | 2717 | 9733 | 58.4 |
| D 12 | serum | 2913 | 2895 | 96,402,567 | 33093 | 2711 | 8819 | 44.2 |
| D 16 | serum | 3875 | 3703 | 119,329,130 | 30794 | 1953 | 4984 | 53.6 |
| iPSCs | serum | 3124 | 3088 | 128,207,617 | 41039 | 2637 | 9689 | 46.1 |
| | Total | 68339 | 65781 | | | | | |
| | | | Average | 38,462 | | | | |
| | | | depth | | | | | |
| | | | per cell: | | | | | |

### Example 3 the Reprogramming Landscape Reveals Relationships Among Biological Features

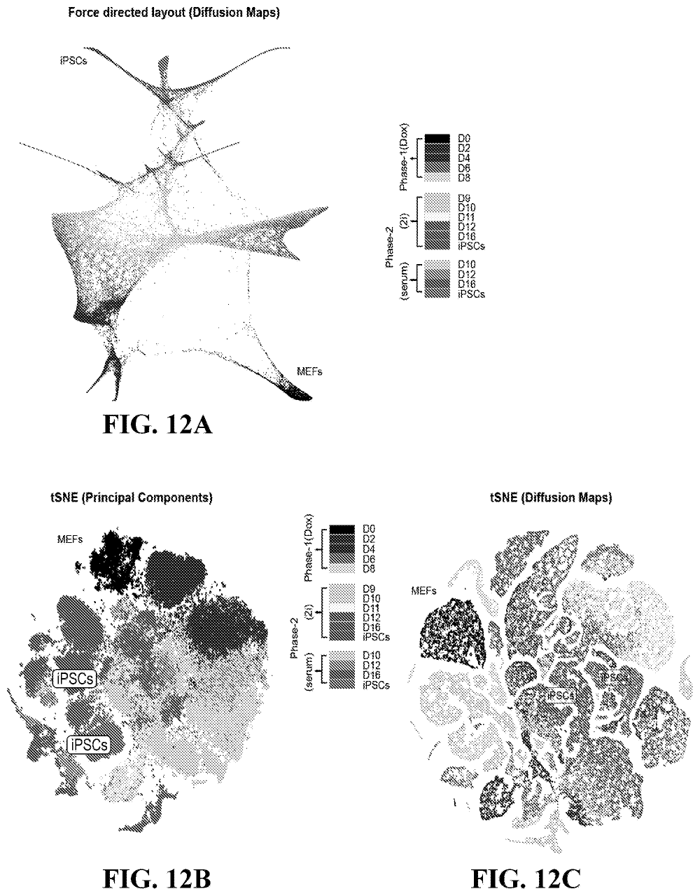

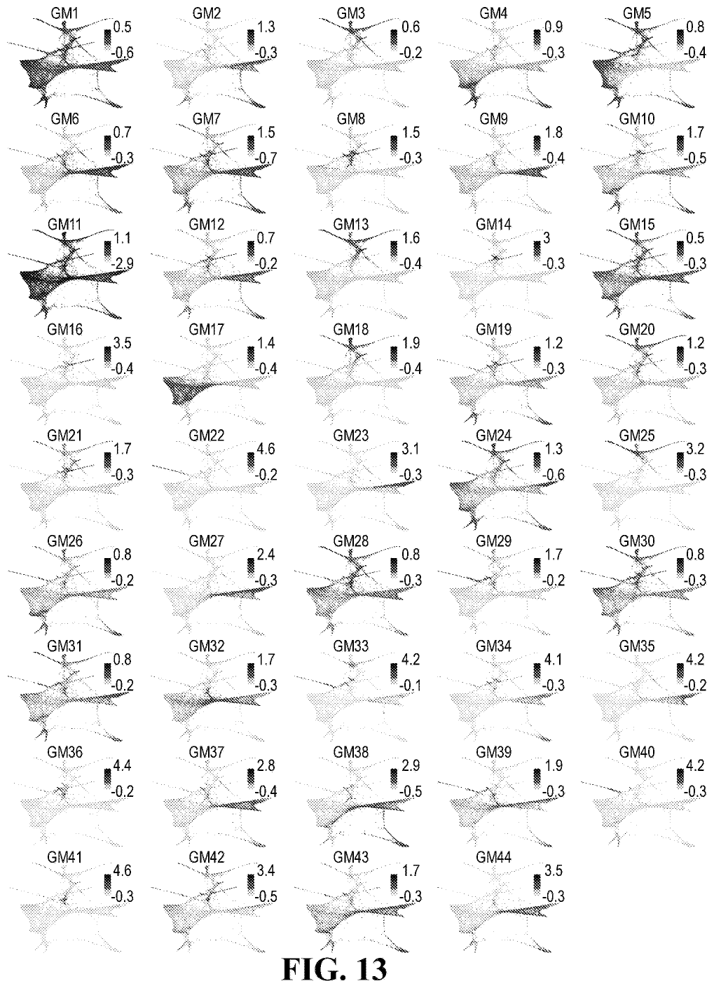

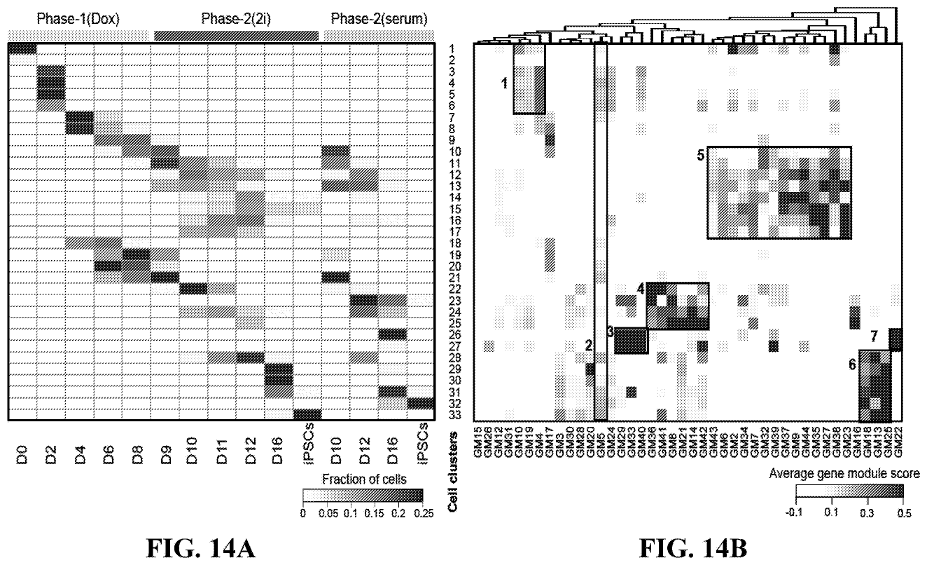

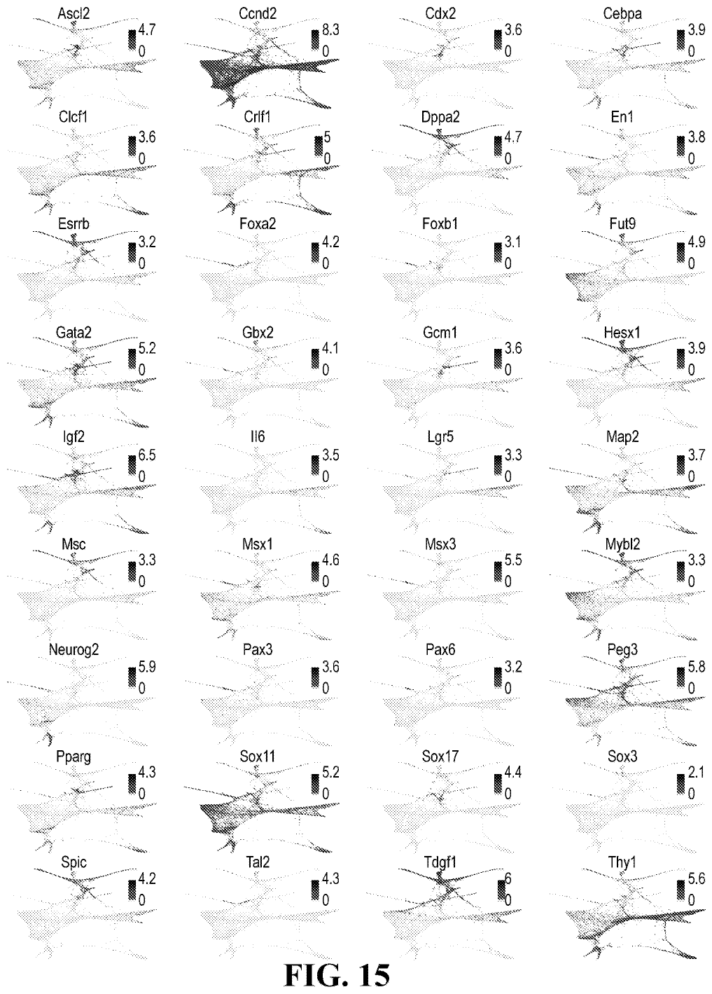

WADDINGTON-OT was used to generate a transport map across the cells in the time course described in the previous example. Based on similarity of expression profiles, the 16,339 detected genes were partitioned into 44 gene modules and the 65,781 cells into 33 cell clusters. Some of the clusters contained cells from more than one time point, reflecting asynchrony in the reprogramming process. The landscape of reprogramming was explored by identifying cell subsets of interest (e.g., successfully reprogrammed cells at day 16, or each of the cell clusters), studying the trajectories to and from these subsets (e.g., characterizing the pattern of gene expression in ancestors at day 8 of successfully reprogrammed target cells at day 16), and considering contemporaneous interactions between them. The analyses were visualized in a two-dimensional embedding using FLE (FIG. 7A), annotated in various ways. FLE reflects better global structures in the data presented herein than other modes of visualization (FIGS. 12A-12C). These annotations include time points and growth conditions (FIGS. 7B,7C), gene modules (FIGS. 13, 14A-14B, Table 1), cell clusters (FIG. 7D, FIG. 14A-14D, Table 9), expression of gene signatures (curated gene sets associated with specific cell types, pathways, and responses, such as MEF identity, proliferation, pluripotency, and apoptosis; FIG. 7E, Table 7), expression of individual genes (FIG. 7F, FIG. 15), and ancestor and descendant distributions (FIGS. 8A-8F). Extensive sensitivity analysis showed that key biological results for the reprogramming data were largely robust to the details of the formulation. Finally, the WADDINGTON-OT landscape was compared to the landscapes produced by various graph-based methods. The results show the following. Cell trajectories start at the lower right corner at day 0, proceed leftward to day 2 and then upward towards two regions identified as the Valley of Stress and the Horn of Transformation (FIG. 7B, FIG. 8A). The Valley is characterized by signatures of cellular stress, senescence, and, in some regions, apoptosis (FIG. 7E); it appears to be a terminal destination. By contrast, the Horn is characterized by increased proliferation, loss of fibroblast identity, a mesenchymal-to-epithelial transition (FIG. 7E), and early appearance of certain pluripotency markers (e.g., Nanog and Zfp42, FIG. 7F), which are predictive features of successful reprogramming (47). Some of the cells in the Horn proceed toward pre-iPSCs by day 12 and iPSCs by day 16, while others encounter alternative fates of placental-like development and neurogenesis (in serum, but not 2i condition; FIGS. 7B, 7C). A more detailed account of the landscape is in the following examples.

TABLE 9

| | Phase-1(Dox) | | | | | Phase-2 (2i) | | | | | | Phase-2 (serum) | | | |
|---|---|---|---|---|---|---|---|---|---|---|---|---|---|---|---|
| Cluster | D 0 | D 2 | D 4 | D 6 | D 8 | D 9 | D 10 | D 11 | D 12 | D 16 | iPSCs | D 10 | D 12 | D 16 | iPSCs |
| 1 | 97.4 | 0.1 | 0.0 | 0.0 | 0.1 | 0.0 | 0.0 | 0.0 | 0.0 | 0.0 | 0.0 | 0.1 | 0.4 | 0.1 | 0.9 |
| 2 | 2.0 | 0.3 | 0.1 | 0.0 | 0.0 | 0.0 | 0.0 | 0.0 | 0.0 | 0.0 | 0.0 | 0.0 | 0.1 | 0.1 | 0.1 |
| 3 | 0.1 | 22.0 | 0.9 | 0.0 | 0.0 | 0.0 | 0.0 | 0.0 | 0.0 | 0.0 | 0.0 | 0.0 | 0.0 | 0.0 | 0.0 |
| 4 | 0.0 | 31.7 | 0.0 | 0.0 | 0.0 | 0.0 | 0.0 | 0.0 | 0.0 | 0.0 | 0.0 | 0.0 | 0.0 | 0.0 | 0.0 |
| 5 | 0.2 | 33.5 | 0.1 | 0.0 | 0.0 | 0.0 | 0.0 | 0.1 | 0.0 | 0.0 | 0.0 | 0.1 | 0.1 | 0.0 | 0.0 |
| 6 | 0.0 | 12.1 | 0.0 | 0.0 | 0.0 | 0.0 | 0.0 | 0.0 | 0.0 | 0.0 | 0.0 | 0.0 | 0.0 | 0.0 | 0.0 |
| 7 | 0.0 | 0.1 | 60.7 | 5.8 | 0.1 | 0.0 | 0.0 | 0.0 | 0.0 | 0.0 | 0.0 | 0.0 | 0.0 | 0.0 | 0.0 |
| 8 | 0.0 | 0.0 | 23.9 | 8.3 | 2.0 | 0.0 | 0.0 | 0.0 | 0.0 | 0.0 | 0.0 | 0.0 | 0.0 | 0.0 | 0.0 |
| 9 | 0.0 | 0.0 | 0.9 | 16.5 | 16.8 | 1.2 | 0.0 | 0.0 | 0.0 | 0.0 | 0.0 | 1.0 | 0.0 | 0.0 | 0.0 |
| 10 | 0.0 | 0.0 | 0.0 | 2.4 | 15.1 | 19.3 | 0.5 | 0.3 | 0.0 | 0.0 | 0.0 | 21.8 | 0.0 | 0.1 | 0.0 |
| 11 | 0.0 | 0.0 | 0.0 | 0.2 | 1.3 | 22.6 | 14.1 | 7.1 | 1.5 | 0.1 | 0.0 | 14.4 | 2.9 | 0.7 | 0.1 |
| 12 | 0.2 | 0.0 | 0.0 | 0.0 | 0.0 | 3.2 | 16.0 | 11.4 | 9.7 | 1.1 | 0.6 | 3.0 | 13.9 | 2.6 | 0.2 |
| 13 | 0.1 | 0.0 | 0.0 | 0.0 | 0.4 | 9.1 | 11.5 | 8.6 | 3.4 | 0.2 | 0.0 | 18.1 | 16.8 | 1.8 | 0.1 |
| 14 | 0.0 | 0.0 | 0.0 | 0.0 | 0.0 | 0.2 | 2.9 | 4.8 | 12.3 | 1.4 | 1.5 | 0.0 | 2.5 | 0.6 | 0.0 |
| 15 | 0.0 | 0.0 | 0.0 | 0.0 | 0.0 | 0.1 | 1.2 | 5.6 | 11.6 | 6.2 | 5.3 | 0.0 | 0.2 | 0.6 | 0.0 |
| 16 | 0.0 | 0.0 | 0.0 | 0.0 | 0.0 | 0.7 | 5.9 | 14.2 | 16.0 | 2.5 | 0.0 | 0.3 | 1.0 | 1.5 | 0.0 |
| 17 | 0.0 | 0.0 | 0.0 | 0.0 | 0.0 | 0.6 | 10.5 | 11.9 | 6.7 | 0.2 | 0.0 | 0.0 | 0.9 | 0.2 | 0.0 |
| 18 | 0.0 | 0.1 | 12.5 | 15.9 | 1.3 | 0.0 | 0.0 | 0.0 | 0.0 | 0.0 | 0.0 | 0.0 | 0.0 | 0.0 | 0.0 |
| 19 | 0.0 | 0.0 | 0.0 | 10.6 | 27.5 | 11.6 | 0.0 | 0.1 | 0.0 | 0.0 | 0.0 | 5.6 | 0.0 | 0.0 | 0.0 |
| 20 | 0.0 | 0.0 | 0.6 | 31.7 | 20.0 | 4.3 | 0.0 | 0.0 | 0.0 | 0.0 | 0.0 | 0.2 | 0.0 | 0.0 | 0.0 |
| 21 | 0.0 | 0.0 | 0.0 | 8.5 | 15.5 | 24.9 | 0.1 | 0.1 | 0.1 | 0.0 | 0.0 | 32.5 | 0.2 | 0.6 | 0.1 |
| 22 | 0.0 | 0.0 | 0.0 | 0.0 | 0.0 | 1.6 | 25.8 | 10.1 | 0.5 | 0.1 | 0.0 | 1.2 | 1.0 | 0.3 | 0.1 |
| 23 | 0.0 | 0.0 | 0.0 | 0.0 | 0.0 | 0.1 | 0.3 | 0.1 | 0.5 | 0.1 | 0.0 | 0.7 | 29.2 | 16.5 | 1.7 |
| 24 | 0.0 | 0.0 | 0.0 | 0.0 | 0.0 | 0.3 | 8.6 | 11.6 | 6.3 | 1.6 | 0.1 | 0.2 | 16.8 | 7.7 | 0.1 |
| 25 | 0.0 | 0.0 | 0.0 | 0.0 | 0.0 | 0.0 | 0.2 | 0.3 | 7.3 | 0.4 | 0.0 | 0.0 | 0.0 | 0.1 | 0.0 |
| 26 | 0.0 | 0.0 | 0.0 | 0.0 | 0.0 | 0.1 | 0.6 | 1.0 | 0.3 | 0.1 | 0.0 | 0.0 | 0.8 | 30.7 | 0.0 |
| 27 | 0.0 | 0.0 | 0.0 | 0.0 | 0.0 | 0.0 | 0.0 | 0.0 | 0.6 | 0.1 | 0.0 | 0.0 | 0.0 | 3.0 | 0.0 |
| 28 | 0.0 | 0.0 | 0.0 | 0.0 | 0.0 | 0.0 | 1.8 | 12.7 | 23.0 | 2.3 | 0.7 | 0.6 | 12.7 | 0.6 | 0.0 |
| 29 | 0.0 | 0.0 | 0.0 | 0.0 | 0.0 | 0.0 | 0.0 | 0.1 | 0.0 | 31.6 | 0.0 | 0.0 | 0.0 | 1.1 | 0.0 |
| 30 | 0.0 | 0.0 | 0.0 | 0.0 | 0.0 | 0.0 | 0.0 | 0.0 | 0.1 | 33.4 | 0.1 | 0.0 | 0.1 | 0.4 | 0.0 |
| 31 | 0.0 | 0.0 | 0.0 | 0.0 | 0.0 | 0.0 | 0.0 | 0.0 | 0.0 | 15.4 | 1.6 | 0.0 | 0.1 | 23.3 | 1.1 |
| 32 | 0.0 | 0.0 | 0.0 | 0.0 | 0.0 | 0.0 | 0.0 | 0.0 | 0.0 | 0.0 | 0.0 | 0.0 | 0.1 | 6.6 | 95.5 |
| 33 | 0.0 | 0.0 | 0.0 | 0.0 | 0.0 | 0.0 | 0.0 | 0.0 | 0.1 | 3.1 | 90.2 | 0.0 | 0.0 | 0.8 | 0.1 |

### Example 4

Predictive markers of reprogramming success are detectable by day 2.

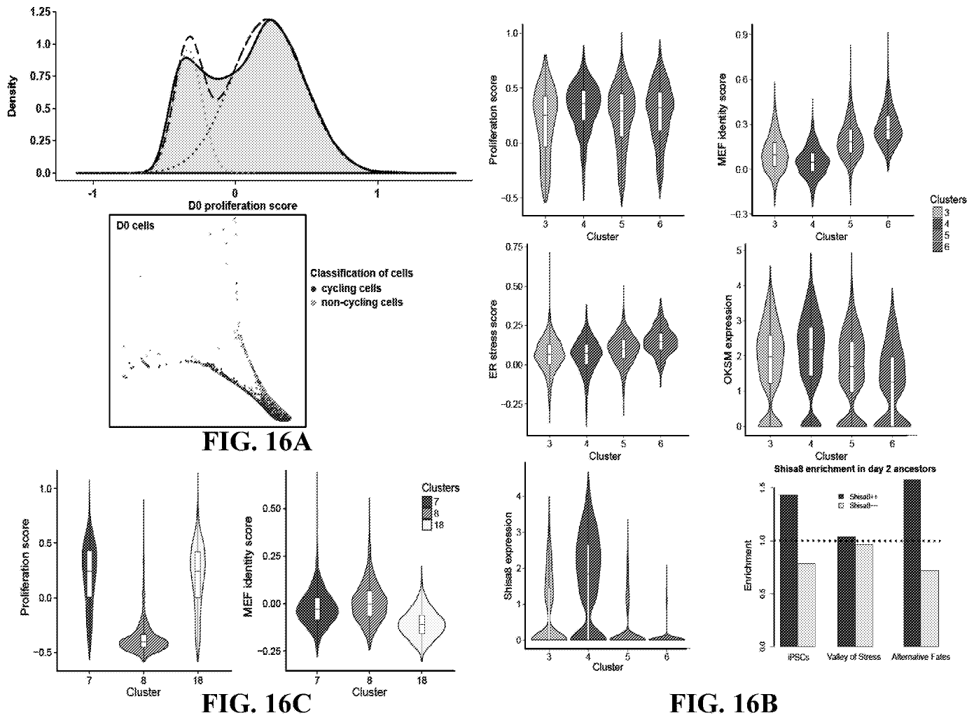

The vast majority (>98%) of cells at day 0 fall into a single cluster characterized by a strong signature of MEF identity, with clear bimodality in the proliferation signature (FIG. 16A). By day 2 after Dox treatment, cells show high levels of expression of the OKSM cassette and have begun to diverge in their responses (clusters 3, 4, 5, 6, FIG. 7D). Overall, they score highly for expression signatures of proliferation, MEF identity, and endoplasmic reticulum (ER) stress (reflecting high secretion in mesenchymal cells) (FIG. 7E).

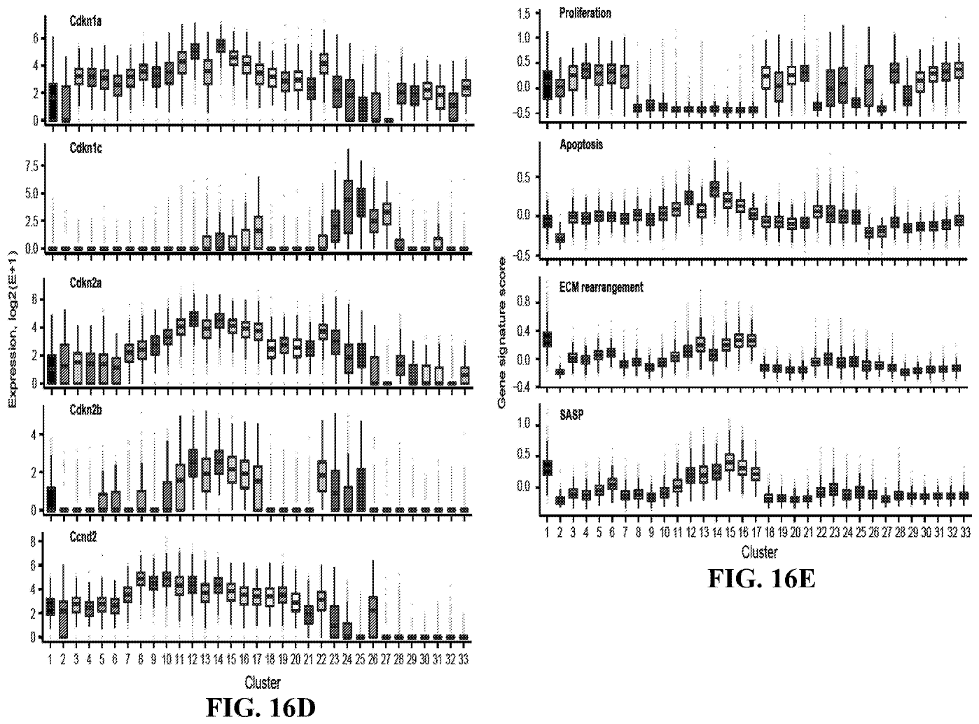

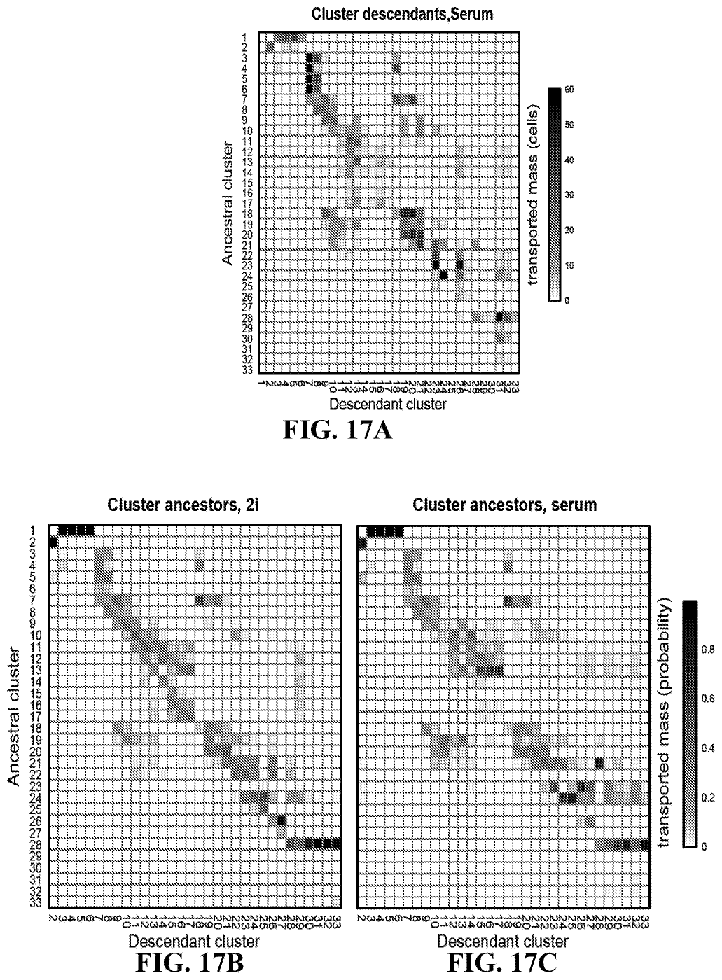

However, the cells exhibit considerable heterogeneity, seen most clearly by comparing the cells in clusters 4 and 6, which vary in their expression signatures and in their fates (FIGS. 8A, 8B and FIGS. 17A-17C). While cells in both clusters are highly proliferative, cells in cluster 4 have begun to lose MEF identity, show lower ER stress, and have higher OKSM-cassette expression, while cells in cluster 6 have the opposite properties (FIGS. 7D, 7E and FIG. 16B). The cells in the two clusters show clear differences in their enrichment in the ancestral distribution of iPSCs (FIG. 8D). The majority (54%) of the day 2 ancestors of iPSCs lie in cluster 4, while only a small fraction (3%) lie in cluster 6. Clusters 4 and 6 also show clear differences in their descendants (FIGS. 8A, 8C and FIG. 17A): the descendants of cells in cluster 6 are strongly biased toward the Valley of Stress (e.g., 81% of Cluster 6 cell descendants are in clusters 8-11 by day 8 vs. 18% for cluster 4), while cluster 4 is strongly biased toward the Horn of Transformation (e.g., 81% in clusters 19-21 vs. 12% for cluster 6).

The strongest difference in gene expression between clusters 4 and 6 was seen for Shisa8 (detected in 67% vs. 3% of cells in clusters 4 and 6, respectively) (FIG. 7F, FIG. 16B) and Shisa8+ cells are enriched among the day 2 ancestors of iPSCs (FIG. 16B). Notably, Shisa8 is strongly associated with the entire trajectory toward successful reprogramming (FIG. 7F): it is expressed in the Horn, pre-iPSCs, and iPSCs, but not in the Valley or in the alternative fates of neurogenesis and placental development. The expression pattern of Shisa8 is similar to, but stronger than, that of Fut9 (FIG. 15), a known early marker of successful reprogramming that synthesizes the surface glyco-antigen SSEA-1 (12). Shisa8 is a little-studied mammalian specific member of the Shisa gene family in vertebrates, which encodes single-transmembrane proteins that play roles in development and are thought to serve as adaptor proteins (48). The analysis suggests that Shisa8 may serve as a useful early predictive marker of eventual reprogramming success and may play a functional role in the process.

### Example 5 Cells in the Valley of Stress Induce a Senescence Associated Secretion Phenotype (SASP)

By day 4, cells display a bimodal distribution of properties that is strongly correlated with their eventual descendants: cells in cluster 8 (low proliferation, high MEF identity, FIG. 7D, E and FIG. 16C) have 95% of their descendants in the Valley (FIGS. 8A, 8B and FIG. 17A), while cells in cluster 18 (high proliferation, low MEF identity, FIGS. 7D, 7E and FIG. 16C) have 94% of their descendants in the Horn (FIGS. 8A, 8B and FIG. 17A and Table 10). Cells in cluster 7 show intermediate properties and have roughly equal probabilities of each fate (FIG. 8A, 8B and FIG. 17A).

TABLE 10

| Cluster | To 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| From 1 | | 0.001 | 0.920 | 0.980 | 0.978 | 0.987 | 0.001 | 0.001 | 0.000 | 0.000 | 0.000 | 0.001 | 0.008 | 0.001 | 0.002 | 0.003 |
| 2 | | 0.790 | 0.000 | 0.003 | 0.003 | 0.000 | 0.000 | 0.000 | 0.000 | 0.000 | 0.000 | 0.000 | 0.000 | 0.000 | 0.000 | 0.000 |
| 3 | | 0.000 | 0.012 | 0.005 | 0.000 | 0.000 | 0.206 | 0.166 | 0.012 | 0.002 | 0.002 | 0.000 | 0.000 | 0.000 | 0.000 | 0.000 |
| 4 | | 0.007 | 0.058 | 0.002 | 0.000 | 0.000 | 0.265 | 0.044 | 0.004 | 0.000 | 0.000 | 0.000 | 0.000 | 0.000 | 0.000 | 0.000 |
| 5 | | 0.106 | 0.008 | 0.003 | 0.006 | 0.003 | 0.293 | 0.298 | 0.004 | 0.000 | 0.000 | 0.001 | 0.000 | 0.000 | 0.000 | 0.000 |
| 6 | | 0.000 | 0.000 | 0.000 | 0.007 | 0.010 | 0.100 | 0.074 | 0.000 | 0.000 | 0.000 | 0.001 | 0.000 | 0.000 | 0.000 | 0.000 |
| 7 | | 0.000 | 0.001 | 0.000 | 0.000 | 0.000 | 0.131 | 0.169 | 0.383 | 0.143 | 0.040 | 0.000 | 0.005 | 0.000 | 0.000 | 0.000 |
| 8 | | 0.000 | 0.000 | 0.000 | 0.000 | 0.000 | 0.003 | 0.240 | 0.171 | 0.126 | 0.018 | 0.000 | 0.005 | 0.000 | 0.000 | 0.000 |
| 9 | | 0.002 | 0.000 | 0.000 | 0.000 | 0.000 | 0.000 | 0.006 | 0.163 | 0.197 | 0.062 | 0.031 | 0.168 | 0.021 | 0.001 | 0.046 |
| 10 | | 0.005 | 0.000 | 0.000 | 0.000 | 0.000 | 0.000 | 0.000 | 0.011 | 0.063 | 0.088 | 0.283 | 0.093 | 0.377 | 0.025 | 0.037 |
| 11 | | 0.004 | 0.000 | 0.000 | 0.000 | 0.000 | 0.000 | 0.000 | 0.002 | 0.001 | 0.031 | 0.216 | 0.081 | 0.211 | 0.085 | 0.065 |
| 12 | | 0.012 | 0.000 | 0.004 | 0.000 | 0.000 | 0.000 | 0.000 | 0.000 | 0.000 | 0.020 | 0.127 | 0.032 | 0.166 | 0.269 | 0.152 |
| 13 | | 0.012 | 0.001 | 0.003 | 0.000 | 0.000 | 0.000 | 0.000 | 0.001 | 0.000 | 0.013 | 0.112 | 0.236 | 0.085 | 0.514 | 0.578 |
| 14 | | 0.002 | 0.000 | 0.000 | 0.000 | 0.000 | 0.000 | 0.000 | 0.000 | 0.000 | 0.003 | 0.017 | 0.002 | 0.028 | 0.037 | 0.017 |
| 15 | | 0.000 | 0.000 | 0.000 | 0.000 | 0.000 | 0.000 | 0.000 | 0.000 | 0.000 | 0.000 | 0.001 | 0.000 | 0.001 | 0.006 | 0.005 |
| 16 | | 0.000 | 0.000 | 0.000 | 0.000 | 0.000 | 0.000 | 0.000 | 0.000 | 0.000 | 0.000 | 0.003 | 0.005 | 0.003 | 0.025 | 0.026 |
| 17 | | 0.000 | 0.000 | 0.000 | 0.000 | 0.000 | 0.000 | 0.000 | 0.000 | 0.000 | 0.000 | 0.003 | 0.003 | 0.003 | 0.026 | 0.027 |
| 18 | | 0.000 | 0.000 | 0.000 | 0.000 | 0.000 | 0.002 | 0.003 | 0.201 | 0.079 | 0.013 | 0.003 | 0.001 | 0.000 | 0.000 | 0.000 |
| 19 | | 0.007 | 0.000 | 0.000 | 0.000 | 0.000 | 0.000 | 0.000 | 0.029 | 0.120 | 0.357 | 0.123 | 0.272 | 0.036 | 0.001 | 0.032 |
| 20 | | 0.000 | 0.000 | 0.000 | 0.001 | 0.000 | 0.000 | 0.000 | 0.018 | 0.172 | 0.270 | 0.047 | 0.052 | 0.001 | 0.000 | 0.002 |
| 21 | | 0.010 | 0.000 | 0.000 | 0.004 | 0.000 | 0.000 | 0.000 | 0.001 | 0.094 | 0.075 | 0.021 | 0.036 | 0.035 | 0.001 | 0.005 |
| 22 | | 0.002 | 0.000 | 0.000 | 0.000 | 0.000 | 0.000 | 0.000 | 0.000 | 0.000 | 0.001 | 0.004 | 0.001 | 0.006 | 0.003 | 0.002 |
| 23 | | 0.027 | 0.000 | 0.000 | 0.000 | 0.000 | 0.000 | 0.000 | 0.000 | 0.001 | 0.005 | 0.004 | 0.001 | 0.021 | 0.004 | 0.003 |
| 24 | | 0.010 | 0.000 | 0.000 | 0.000 | 0.000 | 0.000 | 0.000 | 0.000 | 0.000 | 0.001 | 0.002 | 0.001 | 0.005 | 0.003 | 0.002 |
| 25 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 26 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 27 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 28 | | 0.001 | 0.000 | 0.000 | 0.000 | 0.000 | 0.000 | 0.000 | 0.000 | 0.000 | 0.000 | 0.000 | 0.000 | 0.000 | 0.000 | 0.000 |
| 29 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 30 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 31 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 32 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 33 | | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |

| Cluster | To 17 | 18 | 19 | 20 | 21 | 22 | 23 | 24 | 25 | 26 | 27 | 28 | 29 | 30 | 31 | 32 | 33 |
|---|---|---|---|---|---|---|---|---|---|---|---|---|---|---|---|---|---|
| From 1 | 0.003 | 0.003 | 0.000 | 0.001 | 0.000 | 0.000 | 0.000 | 0.000 | 0.000 | 0.004 | 0.006 | 0.000 | 0.006 | 0.002 | 0.001 | 0.006 | 0.001 |
| 2 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 3 | 0.000 | 0.051 | 0.001 | 0.004 | 0.001 | 0.000 | 0.000 | 0.000 | 0.000 | 0.000 | 0.000 | 0.000 | 0.000 | 0.000 | 0.000 | 0.000 | 0.000 |
| 4 | 0.000 | 0.276 | 0.000 | 0.005 | 0.000 | 0.000 | 0.000 | 0.000 | 0.000 | 0.000 | 0.000 | 0.000 | 0.000 | 0.000 | 0.000 | 0.000 | 0.000 |
| 5 | 0.000 | 0.009 | 0.000 | 0.001 | 0.000 | 0.000 | 0.001 | 0.000 | 0.000 | 0.000 | 0.000 | 0.000 | 0.000 | 0.000 | 0.000 | 0.000 | 0.000 |
| 6 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 7 | 0.000 | 0.578 | 0.183 | 0.340 | 0.044 | 0.000 | 0.000 | 0.000 | 0.000 | 0.000 | 0.000 | 0.000 | 0.000 | 0.000 | 0.000 | 0.000 | 0.000 |
| 8 | 0.000 | 0.008 | 0.008 | 0.001 | 0.005 | 0.000 | 0.000 | 0.000 | 0.000 | 0.000 | 0.000 | 0.000 | 0.000 | 0.000 | 0.000 | 0.000 | 0.000 |
| 9 | 0.026 | 0.004 | 0.047 | 0.003 | 0.073 | 0.011 | 0.001 | 0.005 | 0.000 | 0.000 | 0.000 | 0.001 | 0.000 | 0.001 | 0.000 | 0.000 | 0.000 |
| 10 | 0.058 | 0.000 | 0.033 | 0.001 | 0.069 | 0.080 | 0.065 | 0.026 | 0.015 | 0.001 | 0.001 | 0.009 | 0.001 | 0.003 | 0.000 | 0.001 | 0.000 |
| 11 | 0.111 | 0.000 | 0.003 | 0.001 | 0.006 | 0.005 | 0.000 | 0.000 | 0.000 | 0.007 | 0.012 | 0.001 | 0.012 | 0.004 | 0.003 | 0.012 | 0.001 |
| 12 | 0.084 | 0.000 | 0.000 | 0.000 | 0.000 | 0.014 | 0.000 | 0.000 | 0.000 | 0.025 | 0.046 | 0.002 | 0.043 | 0.015 | 0.009 | 0.041 | 0.004 |
| 13 | 0.650 | 0.000 | 0.001 | 0.000 | 0.001 | 0.015 | 0.000 | 0.000 | 0.000 | 0.037 | 0.066 | 0.003 | 0.057 | 0.020 | 0.011 | 0.055 | 0.005 |
| 14 | 0.006 | 0.000 | 0.000 | 0.000 | 0.000 | 0.003 | 0.000 | 0.000 | 0.000 | 0.006 | 0.010 | 0.000 | 0.010 | 0.004 | 0.002 | 0.010 | 0.001 |
| 15 | 0.002 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 16 | 0.020 | 0.000 | 0.000 | 0.000 | 0.000 | 0.001 | 0.000 | 0.000 | 0.000 | 0.001 | 0.002 | 0.000 | 0.002 | 0.001 | 0.000 | 0.002 | 0.000 |
| 17 | 0.015 | 0.000 | 0.000 | 0.000 | 0.000 | 0.001 | 0.000 | 0.000 | 0.000 | 0.001 | 0.002 | 0.000 | 0.001 | 0.000 | 0.000 | 0.001 | 0.000 |
| 18 | 0.000 | 0.064 | 0.264 | 0.227 | 0.116 | 0.007 | 0.000 | 0.000 | 0.000 | 0.000 | 0.000 | 0.000 | 0.000 | 0.000 | 0.000 | 0.000 | 0.000 |
| 19 | 0.014 | 0.003 | 0.143 | 0.057 | 0.107 | 0.104 | 0.050 | 0.073 | 0.017 | 0.001 | 0.000 | 0.045 | 0.003 | 0.013 | 0.000 | 0.002 | 0.000 |
| 20 | 0.001 | 0.006 | 0.304 | 0.309 | 0.336 | 0.276 | 0.011 | 0.005 | 0.000 | 0.001 | 0.000 | 0.002 | 0.000 | 0.001 | 0.000 | 0.000 | 0.000 |
| 21 | 0.006 | 0.000 | 0.014 | 0.052 | 0.235 | 0.387 | 0.339 | 0.260 | 0.083 | 0.032 | 0.013 | 0.744 | 0.021 | 0.082 | 0.006 | 0.017 | 0.003 |
| 22 | 0.001 | 0.000 | 0.000 | 0.000 | 0.000 | 0.008 | 0.014 | 0.001 | 0.001 | 0.008 | 0.007 | 0.000 | 0.009 | 0.003 | 0.002 | 0.008 | 0.001 |
| 23 | 0.001 | 0.000 | 0.000 | 0.000 | 0.005 | 0.076 | 0.498 | 0.008 | 0.089 | 0.663 | 0.396 | 0.005 | 0.243 | 0.076 | 0.047 | 0.223 | 0.021 |
| 24 | 0.001 | 0.000 | 0.000 | 0.000 | 0.001 | 0.010 | 0.020 | 0.622 | 0.793 | 0.145 | 0.201 | 0.011 | 0.197 | 0.111 | 0.095 | 0.183 | 0.067 |
| 25 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 26 | 0.000 | 0.000 | 0.000 | 0.000 | 0.000 | 0.000 | 0.000 | 0.000 | 0.000 | 0.061 | 0.228 | 0.000 | 0.000 | 0.000 | 0.000 | 0.000 | 0.000 |
| 27 | 0.000 | 0.000 | 0.000 | 0.000 | 0.000 | 0.000 | 0.000 | 0.000 | 0.000 | 0.000 | 0.005 | 0.000 | 0.000 | 0.000 | 0.000 | 0.000 | 0.000 |
| 28 | 0.000 | 0.000 | 0.000 | 0.000 | 0.000 | 0.001 | 0.000 | 0.000 | 0.000 | 0.006 | 0.004 | 0.174 | 0.364 | 0.640 | 0.804 | 0.406 | 0.885 |
| 29 | 0.000 | 0.000 | 0.000 | 0.000 | 0.000 | 0.000 | 0.000 | 0.000 | 0.000 | 0.000 | 0.000 | 0.000 | 0.002 | 0.002 | 0.002 | 0.002 | 0.001 |
| 30 | 0.000 | 0.000 | 0.000 | 0.000 | 0.000 | 0.000 | 0.000 | 0.000 | 0.000 | 0.000 | 0.000 | 0.000 | 0.004 | 0.003 | 0.003 | 0.004 | 0.002 |
| 31 | 0.000 | 0.000 | 0.000 | 0.000 | 0.000 | 0.000 | 0.000 | 0.000 | 0.000 | 0.000 | 0.000 | 0.000 | 0.009 | 0.008 | 0.007 | 0.010 | 0.004 |
| 32 | 0.000 | 0.000 | 0.000 | 0.000 | 0.000 | 0.000 | 0.000 | 0.000 | 0.000 | 0.001 | 0.001 | 0.000 | 0.015 | 0.010 | 0.008 | 0.016 | 0.005 |
| 33 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |

Along the trajectory from cluster 8 to the Valley (days 10-16; FIGS. 8A, 8B and 8E,F), cells show a strong decrease in cell proliferation (FIG. 7E), accompanied by increased expression of various cell-cycle inhibitors, such as Cdkn2a, which encodes p16, an inhibitor of the Cdk4/6 kinase and halts G1/S transition (FIG. 7F), Cdknla (p21), and Cdkn2b (p15) (FIG. 16D), which peaks in the Valley. The cells show increased expression of D-type cyclin gene Ccnd2 (FIGS. 15, 16D) associated with growth arrest (49). A subset of the cells in the Valley (29%; clusters 12 and 14) showed high activity for a gene module that is correlated with a p53 pro-apoptotic signature, compared to all other cells inside the Valley (p-value<10-16, average difference 0.17, Mest) and outside the Valley (p-value<10-16, average difference 0.32, Mest) (FIG. 7E, FIG. 16E).

Cells in the Valley also show activation of signatures of extracellular-matrix (ECM) rearrangement and secretory functions (FIG. 7E, FIG. 16E). Because these properties are consistent with a senescence associated secretory phenotype (SASP), a SASP signature involving 60 genes (50) was used. Cells with this signature appear on day 10 and continue through day 16, consistent with previous reports concerning the timing of onset of stress-induced senescence (50) (FIG. 7E, FIG. 16E).

SASP, which has key roles in wound healing and development that are relevant for reprogramming biology, includes the expression of various soluble factors (including I16), chemokines (including I18), inflammatory factors (including Ifng), and growth factors (including Vegf) that can promote proliferation and inhibit differentiation of epithelial cells (50). Recent reports have suggested that secretion of 116 and other soluble factors by senescent cells can enhance reprogramming (51). Although detectable levels of 116 mRNA were present in only a small fraction of cells both in 2i and serum (0.2%) at days 12 and 16 (0.34% in all cells), the overall SASP signature was evident in 72% of cells in the Valley (vs. 11% elsewhere, primarily in day 0 MEFs). This suggests that the senescent cells in the Valley are likely to have paracrine effects on cells that successfully emerge from the Horn.

### Example 6 Other Cells at Day 4 are Strongly Biased Toward the Horn of Transformation

For the remaining cells at day 4, the forward trajectory is characterized by high proliferation and loss of MEF identity (FIGS. 7B, 7E), and the descendants are strongly biased toward the Horn at day 8 (FIGS. 8A, 8B and FIG. 17A and Table 10). The Horn is distinguished as a point of transformation, where cells that have lost their mesenchymal identity are beginning their transitions to an epithelial fate. As discussed below, a minority of cells in the Horn have begun to express activators of a pluripotency expression program.

Following Dox withdrawal and media replacement on day 8, the cells in the Horn adopt one of four alternative outcomes by day 12 (senescence, neuronal program, placental program, and pre-iPSCs). Roughly half appear to become senescent, migrating through clusters 19 and 10 to the Valley (FIG. 8A). The fate of the remaining cells is strongly influenced by the culture medium. In serum conditions, the proportion of these cells that transition to neuronal, placental and pre-iPSC states is 62%, 13% and 26%, respectively. By contrast, the proportions in 2i condition are 3%, 37% and 59% (Table 10). These results are consistent with the presence in the 2i medium of two small-molecule inhibitors to inhibit differentiation, including one reported to inhibit neuronal differentiation (52).

### Example 7

Neuronal-like and placental-like cells arise during reprogramming.

Two unusual cell populations were analyzed: placental-like cells (clusters 24 and 25, FIGS. 7B, 7D and FIGS. 8A, 8B, 8E, 8F) at day 12 and neural-like cells (clusters 26 and 27, FIGS. 7B, 7D and FIGS. 8A, 8B, 8E, 8F) at day 16. The first group was characterized by high activity of two gene modules enriched in signatures for “epithelial cell differentiation,” “placenta development,” and “reproductive structure development,” while the second group showed high activity of signature for “neuron differentiation,” “axon development,” and “regulation of nervous system development” (Table 1, and FIGS. 7B, 8C, 8E).

Both populations showed a substantial decrease in proliferation (FIG. 7E, FIG. 16E). To explore if a common mechanism was responsible for this change, 98 cell-cycle related genes (53) were examined to identify those that were differentially upregulated in the placenta and neural clusters compared to all other clusters. The most distinctive characteristic was the high expression of Cdknlc, which encodes a cell-cycle inhibitor (p57) that promotes G1 arrest (FIG. 7F) and is required for maintenance of some adult stem cells (54). Other features are also shared between these two alternative lineages and adult stem cells-including the expression of Lgr5, a marker of adult epithelial stem cells in certain tissues (55) (FIG. 15).

The neural-like cells reside in a large “spike” observed at day 16 in serum but not 2i conditions (16% vs. 0.1% of cells), presumably due to differentiation inhibitors in the latter conditions. Cells near the base of the spike (cluster 26, FIG. 7D and FIGS. 8E, 8F) expressed neural stem-cell markers (including Pax6 and Sox2, FIG. 7E, FIG. 15), while cells further out along the spike (cluster 27, FIG. 7D) expressed markers of neuronal differentiation (including Neurog2 and Map2, FIG. 15). The cells thus appear to span multiple stages of neurogenesis along the length of the spike (FIG. 7E).

Analysis of the developmental landscape suggests a potential mechanism for triggering neural differentiation. The ancestors of neural-like cells are largely found in cluster 23 on day 12 (FIGS. 8A, 8F and FIG. 17C and Table 10). At least 19% of cells in cluster 23 express Cntfr, an I16-family receptor that plays a critical role in neuronal differentiation and survival (56) (FIG. 7F); the true proportion is likely to be higher because the gene has low expression. Contemporaneously, senescent cells in the Valley at day 12 express activating ligands (Crlf1 and Clcf1) of Cntfr (FIG. 15). Thus, neural differentiation may be triggered by paracrine signals from senescent cells to Cntfr-expressing cells.

The placental-like cells express high levels of certain imprinted genes on chromosome 7 (Cdknlc, Igf2, Peg3, H19 and Ascl2; FIG. 7F, FIG. 15), as well as TFs (Cdx2 and Sox17) associated with placental development (57, 58) (FIG. 15). They also show elevated levels of an ER stress signature (FIG. 3E), consistent with the secretory nature of placental cells and observations of placental cells in vivo (59). Analysis was performed to address whether the placental-like cells resembled recently described extraembryonic endodermal (XEN) cells from an iPSC reprogramming study (44). It was found that they do not share the distinctive XEN signature of the cells disclosed in that analysis. The proportion of cells in the placental-like population decreased substantially from day 12 to day 16 in 2i conditions, although the optimal-transport analysis could not confidently infer whether the decrease is due to cells dying, being overtaken by faster-growing cells, or transitioning to other fates (FIG. 14A).

The following two tables provide a list of candidate reprogramming factors.

### Example 8

### Trajectory to Successful Reprogramming Reveals a Continuous Program of Gene Activation.

We next studied the trajectory leading to reprogramming (FIGS. 8D, 8E), which passes through pre-iPSCs (cluster 28; FIGS. 8A, 8B) at day 12 en route to iPSC-like cells at day 16. The iPSC-like cells in serum conditions (which reside in cluster 31) closely resemble fully reprogrammed cells grown in serum (cluster 32). By contrast, the iPSC-like cells under 2i conditions are spread across three clusters (cluster 29-31). While the cells in cluster 31 resemble fully reprogrammed cells grown in 2i (cluster 33), those in cluster 29 show distinct properties suggestive of partial differentiation. In particular, cluster 29 shows lower proliferation, lower Nanog expression, and increased expression of genes related to differentiation (FIGS. 7D, 7F).

In contrast to initial descriptions of reprogramming as involving two “waves” of gene expression, the trajectory of successful reprogramming reveals a more complex regulatory program of gene activity (FIG. 9A). By grouping genes according to their temporal patterns of activation in cells on the OT-defined trajectory to successful reprogramming, a rich collection of markers for particular stages can be obtained (FIG. 9A). In particular, 47 genes that appear late in successfully reprogrammed cells (for example, Obox6, Spic, Dppa4) were identified. These genes may provide useful markers to enrich fully reprogrammed iPSCs (Table 2).

### Example 9

Paracrine Signaling from the Valley May Influence Late Stages of Reprogramming.

The simultaneous presence of multiple cell types raises the possibility of paracrine signaling, with secreted factors from one cell type binding to receptors on another cell type. One such potential interaction above, is SASP+ cells in the Valley secreting Crlf1, Clcf1 and neural-like cells on days 12 and 16 expressing the cognate receptor Cntfr.

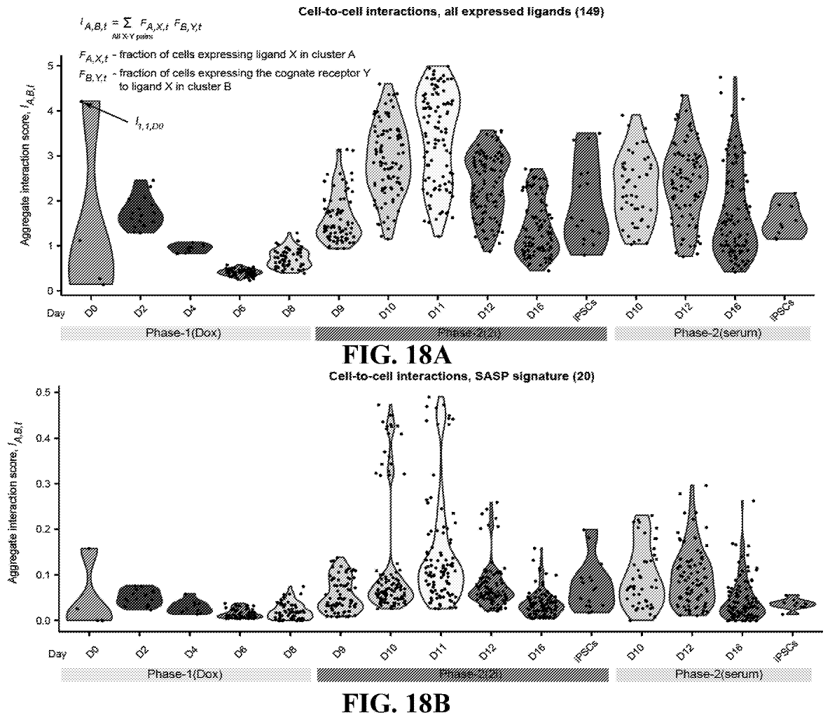

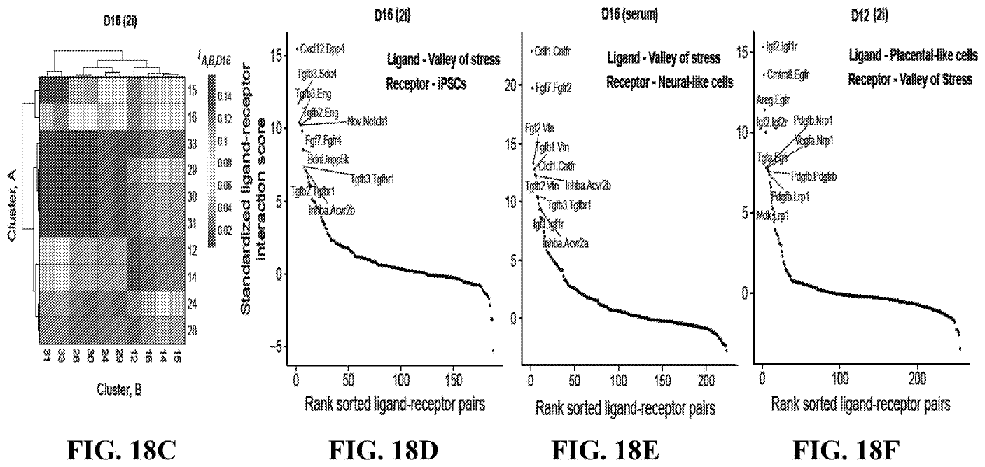

To systematically identify potential opportunities for paracrine signaling, we defined an interaction score, IA,B,X,Y,t, as the product of (1) the fraction of cells in cluster A expressing ligand X and (2) the fraction of cells in cluster B expressing the cognate receptor Y, at time t. Using a curated list of 149 expressed ligands and their associated receptors, we studied potential interactions between all pairs of clusters for each ligand-receptor pair, as well as the aggregate signal across all pairs and across those pairs related to the SASP signature. The potential for paracrine signaling varied sharply across the time course, as well as across cell types. Potential interactions are initially high, as cells with MEF identity retain their secretory functions; drop dramatically by day 6 (FIG. 18A), after cells have lost their MEF identity (FIG. 7B, 7C, 7E); rise steadily from day 8 to day 11, as secretory cells in the Valley emerge; and then drop again from days 12 to 16, as the abundance of cells in the Valley decreases (FIG. 18A). The same pattern is seen when considering only the 20 ligands in the SASP signature (FIG. 18B).

Notably, potential interactions are observed between cells in the Valley and each of iPSC, neural-like and placental-like cells. At day 16, cells in the Valley (clusters 15 and 16) express SASP ligands, while iPSCs (clusters 29-33) express receptors for these ligands (FIG. 18C), with the highest frequency seen for the chemokine Cxcl12 and receptor Dpp4 (FIG. 18D). As noted above, at days 12 and 16, the ligands Crlf1 and Clcf1 cells are expressed in the Valley while their receptor Cntfr is expressed in the neural spike (FIG. 7E, FIG. 18E). The interaction between Cntfr and Crlf1 is ranked as the top interaction among all ligand-receptor pairs (FIG. 18E).

At day 12, many placental-like cells express the ligand Igf2 while cells in the Valley express receptors Igflr and Igf2r (FIG. 18F).

### Example 10

### X-Chromosome Reactivation Follows Activation of Early and Late Pluripotency Genes.

The reversal of X-chromosome inactivation in female cells is known to occur in the late stages of reprogramming and is an example of chromosome-wide chromatin remodeling. A recent study (60) reported that X-reactivation follows the activation of various pluripotency genes, based on immunofluorescence and RNA FISH in single cells. To assess X-reactivation, from scRNA-Seq data, each cell was characterized with respect to signatures of X-inactivation (Xist expression), X-reactivation (proportion of transcripts derived from X-linked genes, normalized to cells at day 0), and early and late pluripotency genes. Along the trajectory to successful reprogramming (but not elsewhere, FIG. 7E), cells at day 12 show strong downregulation of Xist but do not yet display X-reactivation. X-reactivation is complete at day 16, with the signature having risen from 1.0 to ˜1.6, consistent with the expected increase in X-chromosome expression (61). Analysis of the trajectory confirms that activation of both early and late pluripotency genes precedes Xist downregulation and X-reactivation.

### Example 11

### Some Cell Populations are Enriched for Aberrant Genomic Events.

Anaylsis was done to identify other coherent increases or decreases in gene expression across large genomic regions, which might indicate the presence of copy-number variations (CNVs) in specific cells. Particularly, analysis done to identify whole chromosome aberrations, demonstrated that 0.9% of cells showed significant up- or down-regulation across an entire chromosome; the expression-level changes were largely consistent with gain or loss of a single chromosome.

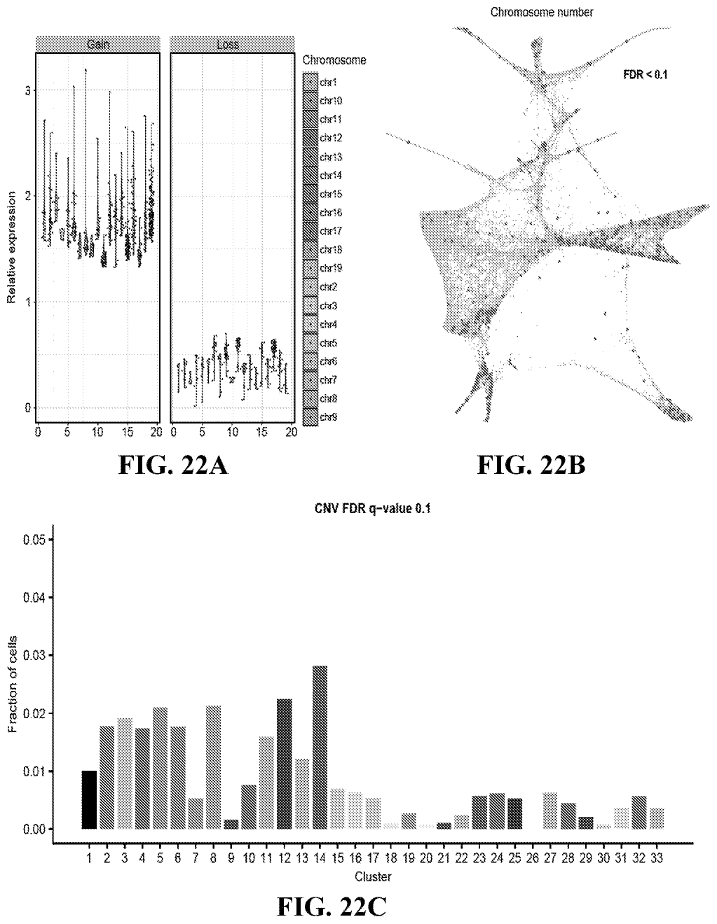

Next, evidence of large subchromosomal events was identified by analyzing regions spanning 25 consecutive housekeeping genes (median size ˜25 Mb). Significant events were found in ˜0.8% of cells. The frequency was highest (2.8%) in cluster 14, consisting of cells in the Valley of Stress enriched for a DNA damage-induced apoptosis signature. The frequency was 2-to-3-fold lower in other cells in the Valley (enriched for senescence but not apoptosis), in cells en route to the Valley (clusters 8 and 11), and in fibroblast-like cells at days 0 and 2. Notably, it was much lower (6-fold) in cells on the trajectory to successful reprogramming (FIGS. 22B, 22C). Direct experimental evidence would be needed to confirm these events, and to clarify if the aberrations were preexisting in the MEF population, or if they accumulated during the course of reprogramming.9

### Example 12

Inferred Trajectories Agree with Experimental Results from Cell Sorting.

To test the accuracy of the probabilistic trajectories calculated for each cell based on optimal transport, results based on the trajectories were compared to experimental data from a recent study of reprogramming of secondary MEFs (16). In that study, cells were flow-sorted at day 10, based on the cell-surface markers CD44 and ICAM1 and a Nanog-EGFP reporter gene, and each sorted population was grown for several days thereafter to monitor reprogramming success. Gene expression profiles were obtained from each population at day 10 and CD44-ICAM1+Nanog+ population at day 15, together with mature iPSCs and ESCs. Reprogramming efficiency was lowest for CD44+ICAM-Nanog-cells, intermediate for CD44-ICAM1+Nanog− and CD44-ICAM1−Nanog+ cells, and highest for CD44-ICAM1+Nanog+ cells.

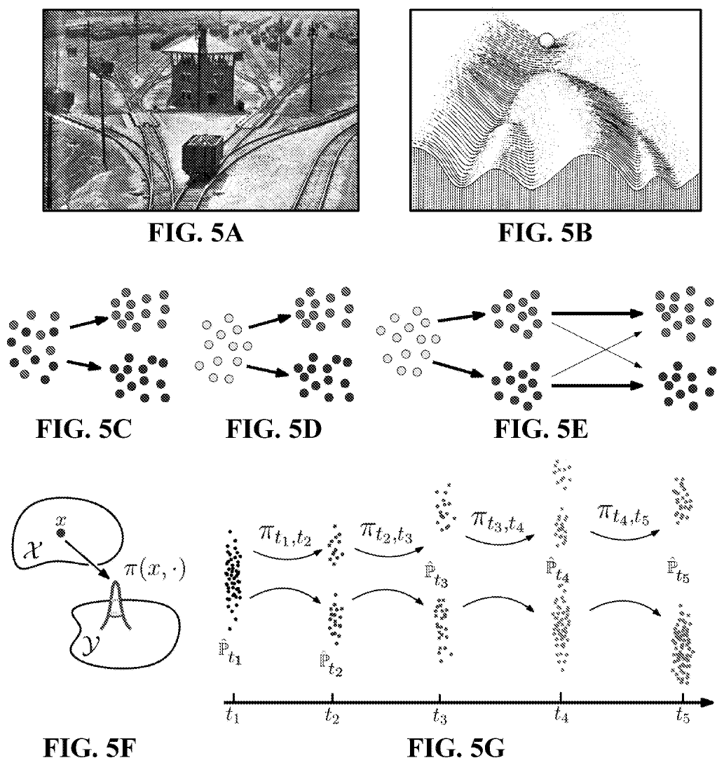

The flow-sorting-and-growth protocol was emulated in silico, by partitioning cells based on transcript levels of the same three genes at day 10 and predicting the fates of each population at day 16 based on the inferred trajectory of each cell in the optimal transport model. The computational predictions showed good agreement with these earlier experimental results (FIG. 5B), with respect to both reprogramming efficiency and changes in gene-expression profiles. In particular, the in silico results showed 93% correlation with results from the earlier study concerning relative reprogramming efficiencies for six categories of sorted cells (p value=0.0023) (FIG. 9B). Notably, the computationally inferred trajectory of double positive cells rapidly transitioned toward iPSCs and continued in this direction through the end of the time course (FIG. 9B). Only one category (CD44-ICAM+Nanog−) differed significantly.

Differences may reflect the fact that experimental protocols were not identical (e.g., the earlier study (16) maintains continuous expression of OSKM and supplements the medium with an ALK-inhibitor and vitamin C).

### Example 13

Inferring Transcriptional Regulators that Control the Reprogramming Landscape.

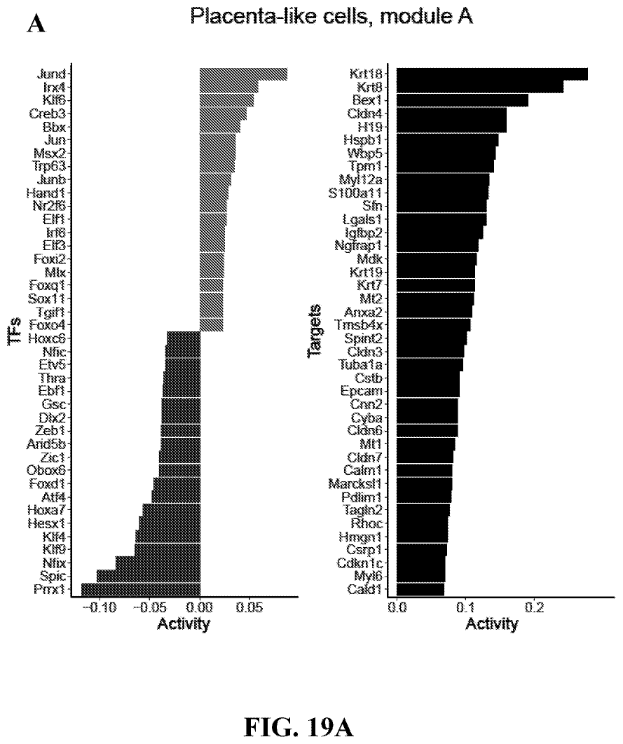

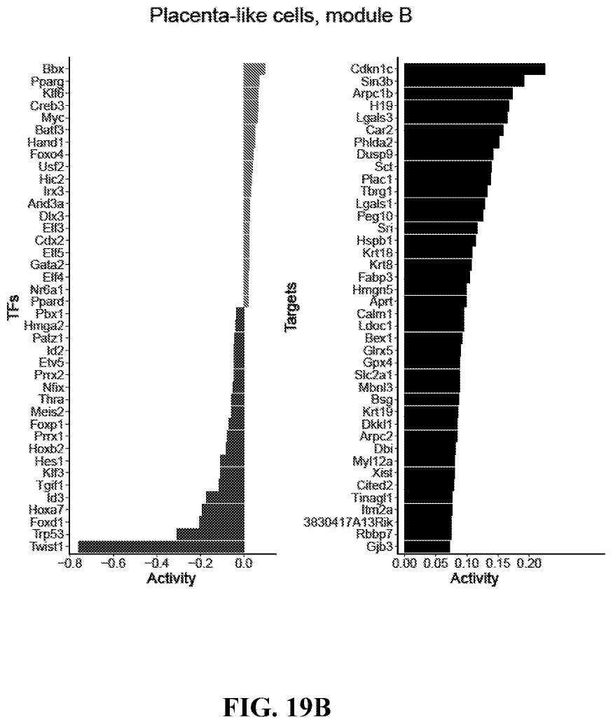

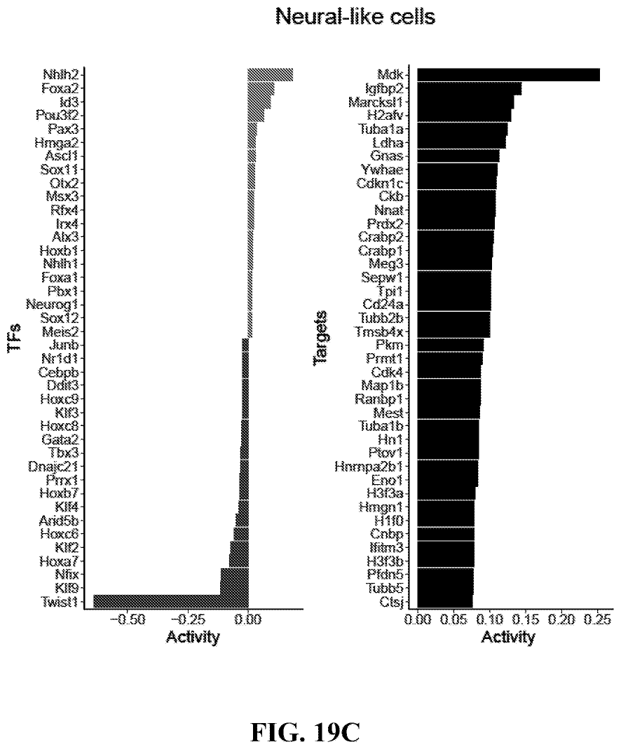

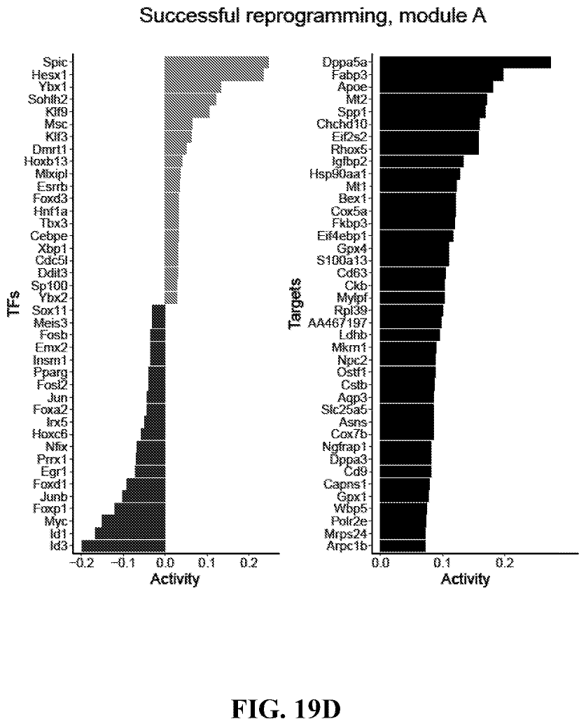

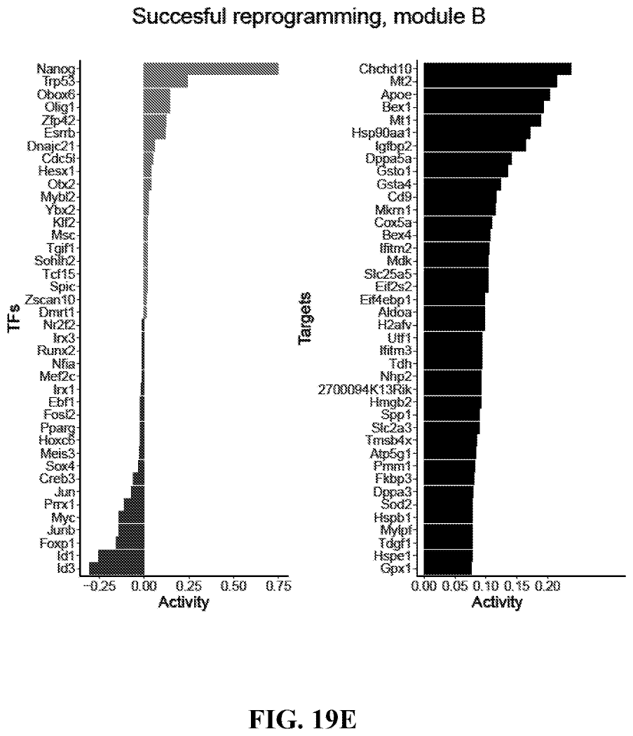

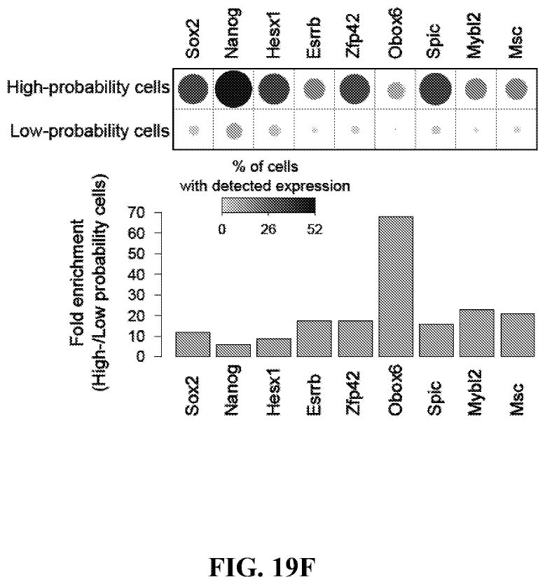

The optimal transport map provides an opportunity to infer regulatory models, based on association between TF expression in ancestors and gene expression patterns in descendants. TFs were identified by two approaches (FIG. 9C): (i) a global regulatory model, to identify modules of TFs and target genes and (ii) enrichment analysis, to identify TFs in cells having many vs.few descendants in a target cell population of interest. Gene regulation along the trajectories to placental-like and neural-like cells was examined (FIG. 19). For placental-like cells, the analysis pointed to 22 TFs (FIGS. 19A, 19B and Table 3). Of the four most enriched (Pparg, Cebpa, Gcm1, and Gata2), all have been reported to play roles in placenta development (62). For example, Gcm1 was detected in 42% of cells at day 10 with a high proportion (>80%) of descendants in the placental-like fate but only 0.7% of those cells with a low proportion (<20%) (57-fold enrichment). For neural-like cells, the analysis pointed to 10 TFs (Pax3, Msx1, Msx3, Sox3, Sox11, Tal2, En1, Foxa2, Gbx2, and Foxb1). All have been implicated in various aspects of neural development (FIG. 19C) (62-70).

Additional analysis focused on identifying TFs that play roles along the trajectory to successful reprogramming (FIG. 9D and FIG. 19D, 19E). The global regulatory model generated two regulatory modules, A and B, with 61 TFs in module A, 16 in module B, and 11 in both (FIGS. 19D, 19E).

Module A involves target genes active across clusters 29-31, while Module B involves target genes that are more active in cluster 31, which contains more fully reprogrammed cells. The TFs in these modules are progressively activated across the trajectory of successful reprogramming. For Module B, the TFs are active in 13% of cells in the Horn on day 8, while target-gene activity is evident (at >80% of the levels observed in iPSCs) in 1.3%, 10%, and 21% of their descendant cells in days 10, 11, and 12 in 2i conditions; the pattern in serum conditions is similar, although with lower overall frequency (11% of cells by day 12). The onset of TFs and target genes in Module A lags by 1-2 days (FIG. 9D).

To identify TFs likely to play a key role in the final stages of reprogramming, we used enrichment analysis to identify TFs enriched in cells at day 12 with a high vs. low proportion (>80% vs.<20%) of successfully reprogrammed descendants and then focused on the intersection of this set with the 66 TFs from the global regulatory analysis above. The analysis pointed to 9 TFs associated with a high probability of success in the late stages of reprogramming (FIG. 19F). Of these, five (Sox2, Nanog, Hesx1, Esrrb, Zfp42) have established roles in regulation of pluripotency (71-73), while the remaining four (Obox6, Spic, Mybl2, and Msc) have not previously been implicated. Among these novel factors, Obox6 stands out as having the greatest enrichment in high-vs. low-probability cells (68-fold, 9.3% vs ˜0.14%) (FIG. 19F).

### Example 14

### Forced Expression of Obox6 Enhances Reprogramming.

Obox6 was identified by the regulatory analysis described herein as strongly correlating to reprogramming success. Obox6 (oocyte-specific homeobox 6) is a homeobox gene of unknown function that is preferentially expressed in the oocyte, zygote, early embryos and embryonic stem cells (74).

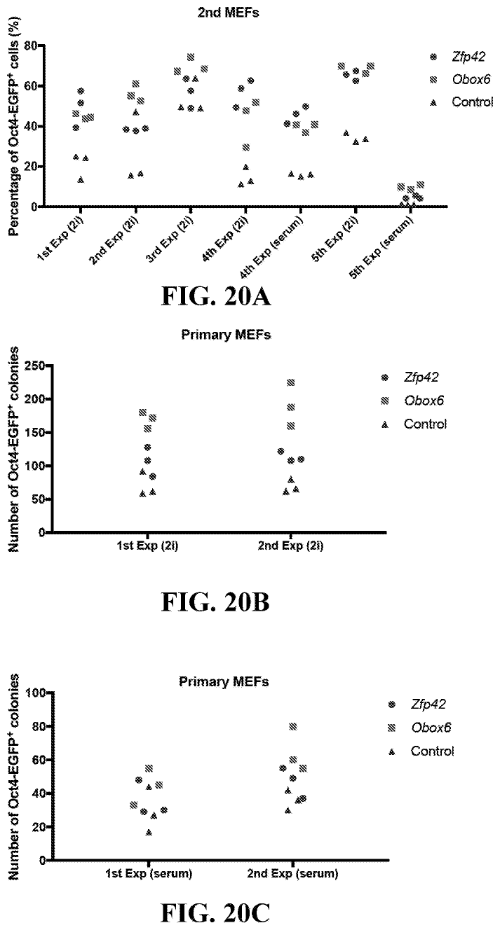

To test whether Obox6 also plays an active role in the process of reprogramming, experiments were performed to address whether expressing Obox6 along with OKSM during days 0-8 can boost reprogramming efficiency. Secondary MEFs were infected with a Dox-inducible lentivirus carrying either Obox6, the known pluripotency factor Zfp42 (73), or no insert as a negative control. Both Obox6 and Zpf42 increased reprogramming efficiency of secondary MEFs by ˜2-fold in 2i and even more so in serum. The results were confirmed in multiple independent experiments (FIGS. 10A and 10B, and FIG. 20). Assays in primary MEFs showed similar increases in reprogramming efficiency (FIG. 20). These results demonstrate the importance of Obox6 in the context of cellular reprogramming.

FIGS. 10A-10C demonstrate the effect of overexpression of Obox6 and Zpf42 on reprogramming efficiency in secondary MEFs. FIGS. 10 A and 10B show bright field and fluorescence images of iPSC colonies generated by lentiviral overexpression of Oct4, Klf4, Sox2, and Myc (OKSM) with either an empty control, Zfp42 or Obox6 expression cassette, in either Phase-1 (Dox)/Phase-2(2i)(A) and Phase-1 (Dox)/Phase-2(serum) (B) conditions (indicated). Cells were imaged at day 16 to measure Oct4-EGFP+ cells. Bar plots representing average percentage of Oct4-EGFP+ colonies in each condition on day 16 are included below the images. Shown are data from one of five independent experiments, with three biological replicates each. Error bars represent standard deviation for the three biological replicates. FIG. 6C is a schematic of the overall reprogramming landscape highlighting: the progression of the successful reprogramming trajectory, alternative cell lineages, and specific transition states (Horn of Transformation). Also highlighted are transcription factors (orange) predicted to play a role in the induction and maintenance of indicated cellular states, and putative cell-cell interactions between contemporaneous cells in the reprogramming system.

### Example 15

### Definition of Gene Signatures

From gene set enrichment analysis of 44 gene modules (Table 1, FIGS. 12A-12C), significant enrichments for terms that shed light on the reprogramming landscape were found. Analysis was done to investigate whether similar expression patterns from well-defined gene signatures could be identified. To investigate this, a list of gene sets from various databases of gene signatures was curated (see Table 11, a list of genes for each gene signature is shown in Table 2). A pluripotency gene signature was determined.

Differential gene expression analysis was performed between two groups of cells: mature iPSCs and cells along the time course D0 to D16, and the top 100 genes with increased expression in mature iPSCs were identified. A proliferation gene signature was obtained by combining genes expressed at G1/S and G2/M phases. For epithelial and neural gene signatures, canonical markers of epithelial and neuronal cell lineage markers, respectively were collected.

TABLE 11
List of gene signatures used in this work. List of
genes for each gene signature are shown in Table 2.

| Gene Signature | Source |
|---|---|
| MEF identity | Mouse Gene Atlas (S29, S30) |
| Pluripotency | this work, iPSCs vs. D0 to D16 cells |
| Proliferation | G1/S and G2/M genes, (S31) |
| ER stress | GO:0034976, Biological Process Ontology |
| Epithelial identity | (S32-S35) |
| ECM rearrangement | GO:0030198, Biological Process Ontology |
| Apoptosis | Hallmark P53 Pathway, MSigDB |
| Senescence | Table 1 in (S36) |
| Neural identity | (S37-S43) |
| Placental identity | Mouse Gene Atlas, (S29, S30) |
| X reactivation | chromosome X |

### Computing Descendant Distributions for Clusters of Cells

The descendant distributions for the 33 clusters of cells, some of which span multiple days were computed. To put each cluster on equal footing, 100 cells in each cluster were initialized. These 100 cells were distributed proportionally over the days represented in the cluster.

For each day d and cluster i, let n_{d}ⁱ denote the number of day d cells in cluster i. We denote the total number of cells in cluster i by Nⁱ Σ_{d}n_{d}ⁱ. With this notation, we initialize

$$100 \times \frac{{n}_{d}^{i}}{{N}^{i}}$$

cells in cluster i on day d and compute the descendant distribution of these cells at the next time point. We denote this descendant distribution by D_{d}ⁱ. We then compute the mass of this descendant distribution residing in each cluster j by summing up the mass D_{d}ⁱ assigns to each cell in cluster j. Finally, to obtain the i, j entry of the cluster-cluster transition table, we sum over d.

This give the total mass transferred from from cluster i to cluster j, per 100 cells initialized in cluster i. We compute this separately for 2i and serum.

### Extraembryonic Gene Signatures

Previous reports have shown that extraembryonic endoderm stem cells (XEN) were induced in the reprogramming process in parallel of reprogramming to iPSCs (S48). To determine if XEN cells were induced in the reprogramming system described herein, the XEN gene signature from in vivo XEN cells, trophoblast and placental gene signatures was analyzed (Table 12). While a small fraction of cells (180 cells) displays a high XEN score at day 16 (under serum condition), a larger fraction of cells in clusters 24 and 25 displays high trophoblast and placental signature scores. This indicates that the alternative placental-like cell lineage does not share the distinctive XEN signature as previously reported.

TABLE 12
List of XEN, trophoblast and placenta gene signatures

| Gene Signature | Genes | Reference |
|---|---|---|
| XEN | Dab2 Fst Pdgfra Pth1r Gatab Foxq1 | (S49) |
| | Fxyd3 Tet3 Sox17 Foxa2 Lama1 Lamb1 | |
| | Gata4 Krt8 | |
| Trophoblast | Ascl2 Bmp4 Bmp8b Cdx2 Elf5 Eomes | (S50) |
| | Esrrb Ets2 Fgfr2 Grn Igf2 Jade1 Lipg | |
| | Pcsk6 Ptpra Smad3 Snai1 Tead4 Tfap2c | |
| | Vav1 Yap1 Gata3 Krt7 Krt18 | |
| Placenta | Table A1 | |

### Example 16

### Identifying Markers for Reprogramming Success

To gain further insights into the mechanisms of reprogramming success, categories of genes that changed their expression in characteristic patterns (FIGS. 5A-5G) along the successful trajectory determined by optimal transport were characterized. Genes that exhibited significant changes along the trajectory (2,872 genes) were clustered using k-means clustering and the number of clusters was determined by the gap statistic (S44). 14 distinct expression patterns among cells that would end up successfully reprogrammed (Table 10) were identified. Genes were divided into two obvious patterns, upregulated (A1 to A10) and downregulated (A11 to A14). After dox induction, a large number of genes that were mainly involved with MEF identify were downregulated. Instead of “two waves” indicated by a previous report (S45), continuous activation patterns after dox induction were observed. In early stage of reprogramming, they were involved with metabolic changes and were targets of Myc (A1 to A3). In late stage (A6 and A7) they were associated with activation of pluripotency networks. Two categories of pluripotency-associated genes were identifed. Genes in category A6 gradually upregulated after dox withdrawal, such as Nanog, Sox2, Dppa3 (early pluripotency-associated genes). Genes in category A7 upregulated after genes in A6, such as Obox6, Dppa4 (late pluripotency-associated genes).

Genes that were upregulated preferentially in cells that were successfully reprogrammed from A6 and A7 were identifed. The fraction of cells in clusters 28 to 33 vs. all other clusters were calculated. By setting a threshold of 1%, genes that were expressed in less than 1% of cells in all other clusters were ranked. 47 genes that were preferentially expressed in the late stage of reprogramming on successful trajectory and were mostly absent from other cells (Table 10) were identified.

### Example 17

### Cell-Cell Interactions

To characterize potential cell-cell interactions between contemporaneous cells during reprogramming, a list of ligands and receptors found in the GO database were collected. The set of ligands (415 genes) is a union of three gene sets from the following GO terms: 1) cytokine activity (GO:0005125), 2) growth factor activity (GO:0008083), and 3) hormone activity (GO:0005179). The set of receptors (2335 genes) is defined by the GO term receptor activity (GO:0004872). Next, a curated database of mouse protein-protein interactions (S46) was used to identify 580 potential ligand-receptor pairs. Two aspects of potential cell-cell interactions in the data were the focus of the analysis: 1) determining global trends in the expression of all potential contemporaneous ligand-receptor pairs across the reprogramming time course and 2) ranking individual ligand-receptor pairs at a specific day and condition. First, an interaction score I_{A,B,X Y,t} as the product of (1) the fraction of cells (F_{A,X,t}) in cluster A expressing ligand X at time t and (2) the fraction of cells (F_{B,Y,t}) in cluster B expressing the cognate receptor Y at time t was defined. Aggregate interaction score I_{A,B,t} was defined as a sum of the individual interaction scores across all pairs:

$${I}_{A , B , t} = {\sum }_{All X Y pairs} {I}_{A , B , X , Y , t} = {\sum }_{Alll X Y pairs} {F}_{A , X , t} {F}_{B , Y , t}$$

The aggregate interaction scores for all combinations of cell clusters in figs. 18A-B were depicted. Second, individual ligand-receptor pairs at a given day and condition between cell subsets of interest were examined. Values of the interaction scores I_{A,B,X,Y,t} are high for ubiquitously expressed ligands and receptors at a given day and may be nonspecific to a pair of cell subsets of interest. Thus, permutations were used to generate an empirical null distribution of interaction scores between two random groups of cells. In each of the 10,000 permutations, two groups R1 and R2 of 100 cells each from time t were selected and the interaction score between the ligand in group R1 and the receptor in group R2 was calculated. Each ligand-receptor interaction score was standardized by taking the distance between the interaction score I_{A,B,x,Y,t} and the mean interaction score in units of standard deviations from the permuted data ((I_{A,B,x,Y,t}−mean(I_{R1,R2,X Y,t})/sd(I_{R1,R2,X,Y,t})). Examples of standardized interaction scores ranked by their values are depicted in FIGS. 18D-F.

### Example 18

### X-Chromosome Reactivation

Analysis was performed to identify X-chromosome reactivation from our scRNA-seq dataset. The set of all detected genes (16,339) was split to X-chromosomal and autosomal genes. Then the mean X/autosome expression ratio for each cell (normalized by the average X/autosome expression ratio at day 0 cells) as a measurement of X-chromosome reactivation was calculated.

The mean X/Autosome expression ratio reached mean value of 1.6 in late stage of reprogramming indicating X-chromosome reactivation. Interestingly, cells in cluster 32 (mature iPSCs in serum) had their X-chromosome inactivated but no Xist expression, which might be due to partial differentiation of iPSCs in serum condition or that the established female iPSCs lost one of their X chromosomes, which happens frequently in serum cultured female ESCs or iPSCs but less often in 2i cultured female ESCs/iPSCs (S47). This was specific to mature iPSCs in serum as day-16 cells in serum exhibited similar X-chromosome reactivation to day 16 cells in 2i

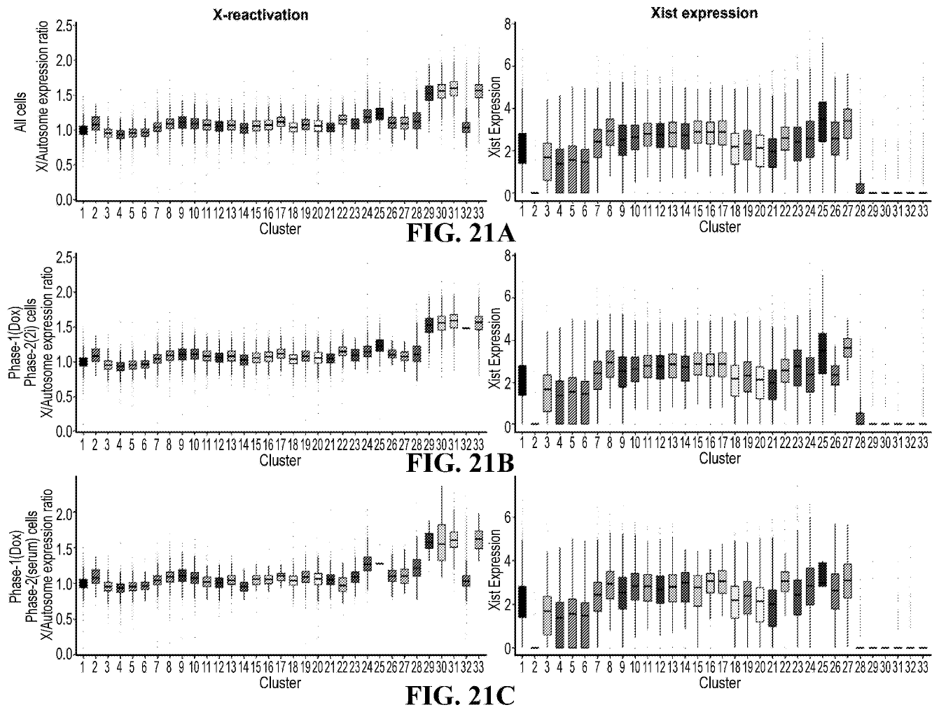

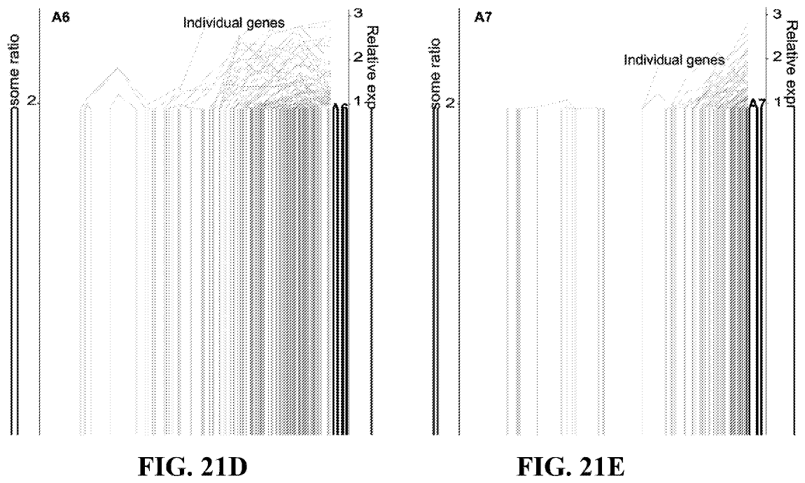

Downregulation of Xist expression (cluster 28, day 12 cells) preceded X-chromosome reactivation (clusters 29,30,31,and 33; day 16, mature iPSCs) (FIGS. 21A-21C). The upregulation of early and late pluripotency genes (activation pattern A6 and A7, respectively) preceded X-chromosome reactivation (FIGS. 21D-21F).

The fraction of cells that activated late pluripotency genes A7 and reactivated the X-chromosome were analyzed. The X/Autosome expression ratio and A7 gene signature score show bimodal distribution across all cells (FIG. 21G and FIG. 21H, respectively). We classified cells to those that had reactivated their X-chromosome if the X/Autosome expression ratio >1.4 and those that induced A7 genes if the A7 average z-score>0.25 (figs. 21G, 21H). Using the above thresholds the fraction of cells in clusters 28-33 that reactivated their X-chromosome and activated the A7 program (Table 13) were calculated. Around a 10-fold difference is observed in the percentage of cells that upregulated A7 genes and reactivated X chromosome in clusters 28 and 32.

TABLE 13
Percentage of cells in clusters 28-33 that exhibited
X-chromosome reactivation and induction of A7 genes.

| Cluster | 28 | 29 | 30 | 31 | 32 | 33 |
|---|---|---|---|---|---|---|
| X/A | 7.6 | 79.3 | 84.2 | 89.1 | 7.2 | 81.9 |
| A7 | 72.9 | 98.9 | 99.7 | 99.1 | 93.3 | 99.1 |

### Example 19

### Identifying Large Chromosomal Aberrations

Methodology. Two types of analysis were performed to detect aberrant expression in large chromosomal regions. First, analysis was performed to identify cells with significant up- or down-regulation at the level of entire chromosomes. Second, analysis was performed to identify cells with significant subchromosomal aberrations spanning windows of 25 consecutive broadly-expressed genes. Empirical p-values and false discovery rates (FDRs) for both analyses were computed by randomly permuting the arrangement of genes in the genome, as described below.

Permutations for both types of analysis are done as follows. In each of 100,000 permutations the labels of genes in the entire dataset were randomly shuffled, while preserving the genomic positions of genes (with each position having a new label each time) and the expression levels in each cell (so that each cell has the same expression values, but with new labels). Either whole chromosome or subchromosomal aberration scores for each cell were calculated. To identify whole-chromosome aberrations scores in each cell, the sum of expression levels in 25Mbp sliding windows along each chromosome, with each window sliding 1Mbp so that it overlaps the previous window by 24Mbp was calculated. For each window in each cell, the Z-score of the net expression, relative to the same window in all other cells was calculated. The fraction of windows on each chromosome with an absolute value Z-score>2 was counted. This fraction serves as the whole-chromosome aberration score for each chromosome in each cell. To assign a p-value to the whole-chromosome score for cellj chromosomej, the empirical probability that the score for cellj chromosomej in the randomly permuted data was at least as large as the score in the original data was calculated.

Subchromosomal aberration scores were computed as follows. The 20% of genes with the most uniform expression across the entire dataset were identified. This is done by calculating the Shannon Diversity (eentropy(gene)) for each gene, and taking the 20% of genes with the largest values. Using these genes, the sum of expression in sliding windows of 25 consecutive genes, with each window sliding by one gene and overlapping the previous window (on the same chromosome) by 24 genes was calculated. In each window, the Z-score relative to all cells at day 0 was calculated. The net subchromosomal aberration score for a cell is calculated as the l2-norm of the Z-scores across all windows. To assign a p-value to the subchromosomal aberration score for celli, the empirical probability that the score for celli in the randomly permuted data was at least as large as the score in the original data was calculated.

For subchromosomal aberration scores chromosomal aberrations (vs. locally coordinated programs of gene expression) were enriched for by excluding recurrent events. Recurrent events were identified by clustering cells based on their aberration profiles (net expression levels across all windows). Clustering was completed by calculating the SVD of all aberration profiles, and performing KMeans clustering on the the top 10 singular vectors (with k=100). For each cluster, we quantified cluster compactness and separation using the silhouette score. Cells that were in compact, well-separated clusters (with a silhouette score>0.08) were removed from consideration for subchromosomal aberrations.

For both types of scores, p-values were used to calculate false discovery rates (FDRs). To identify cells with aberrations at an FDR of q, the largest p-value, \hat{p} was identified, such that \hat{p}N/sum(p<\hat{p}), where N represents the total number of p-values for a score and sum (p<\hat{p}) represents the number of p-values less than p.

Since recurrent aberrations are expected in this setting (due to clonal expansion) cells based on clustering recurrent patterns were not removed. Applied to these data, this method detected aberrations in 35% of malignant cells (classified in the original study as containing significant copy number variation) and 0% of non-malignant cells (FDR 5%). This demonstrates the specificity and conservative nature of the approach.

Results. The results of this analysis are displayed in FIGS. 22A-22C. In analysis designed to look for whole chromosome aberrations, it was found that 0.9% of cells showed significant up- or downregulation across an entire chromosome; the expression-level changes were largely consistent with gain or loss of a single chromosome (A11A). Next, analysis performed to look for evidence of large subchromosomal events, found significant events in 0.8% of cells. The frequency was highest (2.8%) in cluster 14, consisting of cells in the Valley of Stress enriched for a DNA damage-induced apoptosis signature. The frequency was 2-to-3-fold lower in other cells in the Valley (enriched for senescence but not apoptosis), in cells en route to the Valley (clusters 8 and 11), and in fibroblast-like cells at days 0 and 2. Notably, it was much lower (6-fold) in cells on the trajectory to successful reprogramming (FIGS. 22B, 22C). Direct experimental evidence would be needed to confirm these events, and to clarify if the aberrations were preexisting in the MEF population, or if they accumulated during the course of reprogramming.

### Example 20

Forced expression of transcriptional regulators enhances reprogramming.

To test whether any of the transcriptional regulators provided in Tables 2, 3 and 4, for example, Obox6, Spic, Zfp42, Sox2, Mybl2, Msc, Nanog, Hesx1 and Esrrb, play an active role in the process of reprogramming, experiments are performed to address whether expressing these transcription regulators along with OKSM during days 0-8 can boost reprogramming efficiency. Secondary MEFs or primary MEFS are infected with a Dox-inducible lentivirus carrying any one of the transcription regulators provided in Tables 2, 3 and 4, the known pluripotency factor Zfp42 (73), or no insert as a negative control. Reprogramming efficiency is assessed in 2i or in serum. Multiple independent experiments are performed. An increase in reprogramming efficiency by a transcriptional regulator identifies the regulator as important in the context of cellular reprogramming.

Reprogramming efficiency is assessed by analyzing bright field and fluorescence images of iPSC colonies generated by lentiviral overexpression of Oct4, Klf4, Sox2, and Myc (OKSM) with either an empty control, Zfp42 or an expression cassette for any one of the transcription regulators provided in Tables 2, 3 and 4, in either Phase-1(Dox)/Phase-2(2i)(A) and Phase-1(Dox)/Phase-2(serum). Cells are imaged at day 16 to measure Oct4-EGFP+ cells. Bar plots representing average percentage of Oct4-EGFP+ colonies in each condition on day 16 are generated. Error bars represent standard deviation for biological replicates.

### Example 20

Reconstruction of developmental landscapes by optimal-transport analysis of single-cell gene expression across time sheds light on reprogramming

Here, we introduced Waddington-OT, a new approach for studying developmental time courses to infer ancestor-descendant fates and model the regulatory programs that underlie them. We applied Waddington-OT to reconstruct the landscape of reprogramming from 315,000 scRNA-seq profiles, collected mostly at half-day intervals across 18 days. We revealed a wider range of developmental programs than previously recognized. Cells gradually adopted either a terminal stromal state or a mesenchymal-to-epithelial transition state. The latter gave rise to populations related to pluripotent, extra-embryonic, and neural cells, with each harboring multiple finer subpopulations. We predicted transcription factors controlling various fates, of which we showed that Obox6 enhanced reprogramming efficiency. We also found rich potential for paracrine signaling. Our approach shedded new light on the process and outcome of reprogramming and provided a framework applicable to diverse temporal processes in biology.

In the mid-20th century, Waddington introduced two metaphors that shaped biological thinking about cellular differentiation during development: first, trains moving along branching railroad tracks and, later, marbles following probabilistic trajectories as they roll through a developmental landscape of ridges and valleys (Waddington, 1936, 1957). Empirically reconstructing and studying the actual landscapes, fates and trajectories associated with cellular differentiation and de-differentiation—such as in organismal development, long-term physiological responses, and induced reprogramming—requires general approaches to answer questions such as: What classes of cells are present at each stage? What was their origin at earlier stages? What are their likely fates at later stages? What genetic regulatory programs control their dynamics? To what extent are events synchronous vs. asynchronous? To what extent are they stochastic vs. deterministic? Is there only a single path to a given fate, or are there multiple developmental paths?

Traditional approaches based on bulk analysis of cell populations were not well suited to addressing these questions, because they did not provide general solutions to two challenges: discovering the cell classes in a population and tracing the development of each class. Progress had historically relied on ad hoc approaches for each question asked (e.g., sorting and following the development of a particular cell class by using an antibody to a class-specific cell-surface protein or a reporter construct).

The first challenge has recently been largely solved by the advent of single-cell RNA-Seq (scRNA-Seq) (Klein et al., 2015; Kumar et al., 2014; Macosko et al., 2015; Ramskold et al., 2012; Shalek et al., 2013; Tanay and Regev, 2017; Tang et al., 2009; Wagner et al., 2016), which allowed cell classes to be discovered based on their expression profiles. The second challenge remained a work-in-progress. ScRNA-seq now offered the prospect of empirically reconstructing developmental trajectories based on snapshots of expression profiles from heterogeneous cell populations undergoing dynamic transitions (Bendall et al., 2014; Marco et al., 2014; Setty et al., 2016; Tanay and Regev, 2017; Trapnell et al., 2014; Wagner et al., 2016). But, to trace the trajectories of cell classes, one may connect the discrete ‘snapshots’ produced by scRNA-Seq into continuous ‘movies.’ At least at present, one may not be able to follow expression profiles of the same cell and its direct descendants across time because current methods may destroy cells to profile their state. While various approaches have been developed to record information about cell lineage, they currently provide only very limited information about a cell's state at all earlier time points (Daniel T. Montoro et al., 2018; Kester and van Oudenaarden, 2018; McKenna et al., 2016).

Comprehensive studies of cell trajectories thus relied heavily on computational reconstruction of paths in gene-expression space. Pioneering work introduced various methods to infer trajectories (Bendall et al., 2014; Cannoodt et al., 2016; Haghverdi et al., 2015; Matsumoto and Kiryu, 2016; Qiu et al., 2017; Rashid et al., 2017; Rostom et al., 2017; Setty et al., 2016; Street et al., 2017; Trapnell et al., 2014; Weinreb et al., 2017; Welch et al., 2016; Zwiessele and Lawrence, 2016). Profiles of heterogeneous populations can provide information about the temporal order of asynchronous processes-enabling cells to be ordered in pseudotime along trajectories, based on their state of differentiation (Bendall et al., 2014). Some approaches used k-nearest neighbor graphs (Bendall et al., 2014) or binary trees (Trapnell et al., 2014) to connect cells into paths. More recently, diffusion maps have been used to order cell-state transitions, by assigning cells to densely populated paths in diffusion-component space (Haghverdi et al., 2015; Haghverdi et al., 2016). Each such path was interpreted as a transition between cellular fates, with trajectories determined by curve fitting and cells pseudotemporally ordered based on the diffusion distance to the endpoints of each path. Recent work has grappled with incorporating branching paths, which were critical for understanding developmental decisions, and have been applied to analyze whole-organism development in zebrafish, frog, and planaria (Briggs et al., 2018; Farrell et al., 2018; Fincher et al., 2018; Plass et al., 2018; Wagner et al., 2018).

While these approaches have shed important light on various biological systems, many important challenges remain. First, most methods neither directly modeled nor explicitly leveraged the temporal information in a developmental time course (Weinreb et al., 2017) because they were designed to extract information about stationary processes (such as adult stem cell differentiation or the cell cycle) in which all stages existed simultaneously across a single population of cells. However, with the rapidly decreasing cost of scRNA-Seq, time-courses may soon be commonplace. Second, many methods model trajectoried in the language of graph theory which imposesed strong structural constraints on the model, such as one-dimensional trajectories (“edges”) and zero-dimensional branch points (“nodes”). Yet, some biological systems may show a gradual divergence of fates that were not captured well by these models (Briggs et al., 2018; Farrell et al., 2018; Wagner et al., 2018). Third, few methods were able to account for cellular growth and death during development. One method capable of modeling nonuniform cellular growth rates was Population Balance Analysis (Weinreb et al., 2017). However, this method assumed the population of cells is in equilibrium, and therefore it was not suited for analyzing dynamical systems where the distribution of cells changed over time.

One case in point was the challenge of understanding cellular reprogramming-such as converting fibroblasts to induced pluripotent stem cells (iPSCs) or trans-differentiating one mature cell type into another. These non-natural processes involved the transient overexpression of a set of transcription factors (TFs) designed to push a cell out of its current state and toward a new fate, even in the absence of the usual developmental context. Reprogramming had great therapeutic potential, but it still tends to be slow, inefficient, and asynchronous (Takahashi and Yamanaka, 2016). Single-cell analysis of trajectories during reprogramming could shed light on questions such as: What is the full range of cell classes that arise during reprogramming? What are the developmental paths that lead to reprogramming and to any alternative fates? Which cell intrinsic factors and cell-cell interactions drive progress along these paths? To what extent do cells activate normal developmental programs vs. unnatural hybrid programs? Can the programs that are activated provide information about the normal developmental landscape? Can the information gleaned be used to improve the efficiency of reprogramming toward a desired destination?

In particular, reprogramming of fibroblasts to induced pluripotent stem cells (iPSCs), as pioneered by Yamanaka (Hou et al., 2013; Shu et al., 2013; Takahashi and Yamanaka, 2006; Yu et al., 2007), has been largely characterized to date by a combination of fate-tracing of cells based on a handful of markers (e.g., Thy1 and CD44 as markers of the fibroblast state, and ICAM1, Oct4, and Nanog as markers of successful reprogramming), together with RNA- and chromatin-profiling studies of bulk cell populations (Buganim et al., 2012; Hussein et al., 2014; O'Malley et al., 2013; Polo et al., 2012; Tonge et al., 2014). With limited cellular resolution, the profiling studies have provided only coarse-grained analyses, such as describing two “transcriptional waves,” with gain of proliferation and loss of fibroblast identity followed by transient activation of developmental regulators and gradual activation of embryonic stem cell (ESC) genes (Polo et al., 2012). Some studies (Mikkelsen et al., 2008; O'Malley et al., 2013; Parenti et al., 2016), including from our own group (Mikkelsen et al., 2008), have noted strong upregulation of several lineage-specific genes from unrelated lineages (e.g., neurons), but it has been unclear whether this reflects coherent differentiation of specific cell types or disorganized gene expression (Kim et al., 2015; Mikkelsen et al., 2008). Most studies that used single-cell methods to study genetic reprogramming have involved few genes or few cells (Buganim et al., 2012, Kim et al., 2015). Recently, a study (Zhao et al., 2018) profiled ˜36,000 cells during chemical reprogramming, but focused only on a single bifurcation separating successful and failed trajectories.

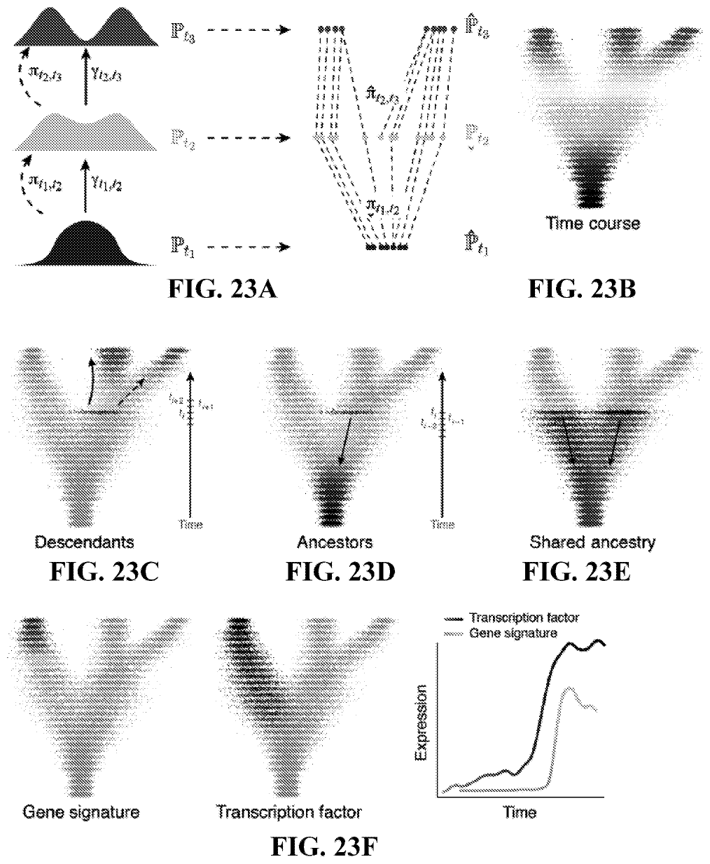

Here, we described a framework, implemented in a method called Waddington-OT, that aimed to capture the notion that cells at any time were drawn from a probability distribution in gene-expression space and cells at any time and position within the landscape had a distribution of both probable origins and probable fates (FIGS. 23A-23F). It then used scRNA-seq data collected across a time-course to infer how these probability distributions evolved over time, by using the mathematical approach of Optimal Transport (OT). We applied and tested this framework in the context of scRNA-seq data we profiled from more than 315,000 cells, sampled across a dense time course over 18 days under two different reprogramming conditions. We found that reprogramming unleashed a much wider range of developmental programs and subprograms than previously recognized, resulting in multiple large distinct populations of cells related to pluripotent, extraembryonic, neural, and stromal cells, with evidence for large-scale genomic amplifications and deletions in trophoblast-like and stromal-like cells. Within each population, there were subsets with distinct programs associated with specific cell types in vivo, including programs associated with 2-, 4-, 8-, 16-, and 32-cell stage embryos; with several distinct types of trophoblasts and primitive endoderm; with astrocytes, oligodendrocytes, and neurons; and with a wider range of stromal cells than MEFs. Trajectory analysis with Waddington-OT showed that differentiation among these classes occurred gradually, including an early gradual transition to either stroma-like cells or a mesenchymal-to-epithelial transition state, with the latter state serving as the ancestor population of both eventual iPSC-like cells and extraembryonic and neural. These differentiation fates were predicted by various sets of TFs, including well studied factors and others not previously implicated. We tested one TF found by our analysis to be associated with pluripotency and showed that it enhanced reprogramming efficiency. Finally, we also found evidence for potential paracrine interactions between the stromal cells and other cell types, which may be important cell extrinsic forces in reprogramming, and for genomic aberrations in certain cells types, with different features in stromal cells and trophoblasts.

Results

Reconstruction of Probabilistic Trajectories by Optimal Transport

A goal of the study was to learn the relationship between ancestor cells at one time point and descendant cells at another time point: given that a cell has a specific expression profile at one time point, where will its descendants likely be at a later time point and where are its likely ancestors at an earlier time point? To this end, we modeled a differentiating population of cells as a time-varying probability distribution (i.e., stochastic process) on a high-dimensional gene expression space. By sampling this probability distribution Pₜ at various time points t, we aimed to infer how the differentiation process it modeled evolves over time (FIG. 23A). By sampling a large number of cells at a given time point, we approximated the distribution at that time point. However, this alone did not tell us the ancestor or descendant relationships between cells at different time points: Because different cells were sampled at different time points, we lost this temporal coupling of the stochastic process Pₜ that specified the joint distribution of expression between pairs of time points. In the absence of any constraint on cellular transitions (e.g., if cells may “jump” about gene-expression space arbitrarily rapidly), we could not infer the temporal coupling. But if we assumed that, over sufficiently short time periods, cells could only move relatively short distance, we could infer the temporal coupling by using the classical mathematical technique of optimal transport (FIG. 23A, Methods).

Optimal transport was originally developed by Monge in 1781 to redistribute earth for the purpose of building fortifications with minimal work (Villani, 2008). In the 1940s, Kantorovich generalized it to identify an optimal coupling of probability distributions via linear programming (Kantorovitch, 1958). This classical linear program minimized the total squared distance that earth travels, subject to conservation of mass constraints. Recent work, which added entropic regularization, dramatically accelerated the numerical computation of large-scale optimal transport problems (Chizat et al., 2017; Cuturi, 2013).

However, matching cells to their descendants differed in one important aspect: unlike earth or particles, cells can proliferate. We therefore modified the classical conservation of mass constraints to accommodate cell growth and death. In particular, we allowed the mass of cells to grow as cells proliferate and shrink as cells die (STAR Methods). By leveraging techniques from unbalanced transport (Chizat et al., 2017), we automatically learned cellular growth and death rates, initializing with prior estimates from signatures of cellular proliferation and apoptosis (STAR Methods).

Using optimal transport, we calculated couplings between consecutive time points and then inferred couplings over longer time-intervals by composing the transport maps between every pair of consecutive intermediate time points. We noted that the optimal-transport calculation (i) implicitly assumed that a cell's fate depended on its current position but not on its previous history (i.e., the stochastic process is Markov) and (ii) captured only the time-varying components of the distribution, rather than processes at dynamic equilibrium. We returned to these points in the Discussion.

We defined trajectories in terms of “descendant distributions” and “ancestor distributions” as follows. For any set C of cells at time ti, its “descendant distribution” at a later time ti+1 referred to the mass distribution over all cells at time ti+1 obtained by transporting C according to the transport maps (FIG. 23C). Branching events, for example, were revealed by the (potentially gradual) emergence of bimodality in the descendant distribution (FIG. 23C). Conversely, its “ancestor distribution” at an earlier time ti−1 was defined as a mass distribution over all cells at time ti−1, obtained by transporting C in the opposite direction (that is, as though one “rewinds” time) (FIG. 23D). Shared ancestry between two cell sets at ti was revealed by convergence of the ancestor distributions (FIG. 23E). The “trajectory from C” referred to the sequence of descendant distributions at each subsequent time point, and the trajectory to C similarly referred to the sequence of ancestor distributions (FIGS. 23C, 23D). For convenience below, we sometimes referred simply to the ‘ancestors, ‘descendants’, and ‘trajectories’ of cells. These terms referred to probability distributions over a set of observed cells that served as proxies for the actual ancestors or descendants. In summary, we used the inferred coupling to calculate a distribution over representative ancestors and descendants at any other time. We then determined the expression of any gene or gene signature along a trajectory by computing the mean expression level weighted by the distribution over cells at each time point.

To identify TFs that regulated the trajectory, we inferred regulatory models by sampling cells from the joint distribution given by the couplings. We developed two approaches: one used ‘local’ enrichment analysis, identifying TFs that were enriched in cells having many vs. few descendants in the target cell population; a second built a global regulatory model, composed of modules of TFs and modules of target genes, to predict expression levels of target gene signatures (FIG. 23F, left) at later time points from expression levels of TFs at earlier time points (FIG. 23F, middle, right).

We implemented our approach in a method, Waddington-OT, for exploratory analysis of developmental landscapes and trajectories, including a public software package (STAR Methods). The method included: (1) Performing optimal-transport analyses on scRNA-seq data from a time course, by calculating optimal-transport maps and using them to find ancestors, descendants and trajectories; (2) Inferring regulatory models that drive the temporal dynamics by sampling pairs of cells from the joint distribution specified by the OT couplings; (3) Visualizing the developmental landscape in two dimensions, by using Force-Directed Layout Embedding (FLE) to visualize the graph of nearest neighbor relationships in diffusion component space (Jacomy et al., 2014; Weinreb et al., 2016; Zunder et al., 2015), and (4) annotating the landscape by cell types, ancestors, descendants, trajectories, gene expression patterns, and other features.

A Dense Experimental scRNA-Seq Time Course of iPS Reprogramming

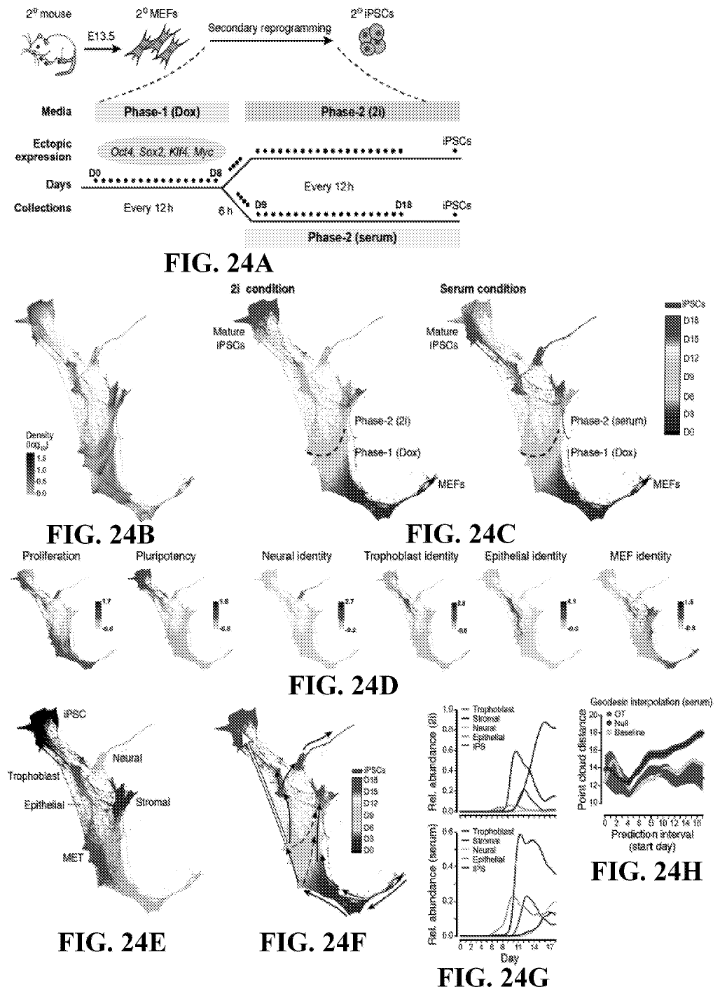

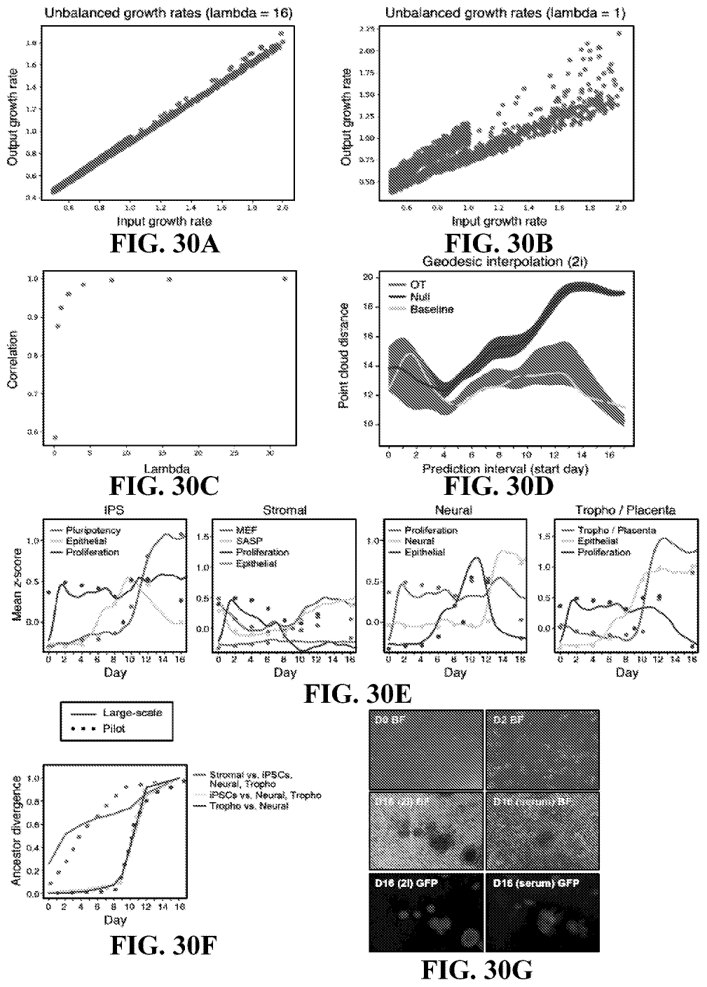

To study the trajectories of reprogramming, we generated iPSCs via a secondary reprogramming system (FIG. 24A), which is more efficient than derivation of iPSCs by primary infection (Stadtfeld et al., 2010). We obtained mouse embryonic fibroblasts (MEFs) from a single female embryo homozygous for ROSA26-M2rtTA, which constitutively expresses a reverse transactivator controlled by doxycycline (Dox), a Dox-inducible polycistronic cassette carrying Pou5f1 (Oct4), Klf4, Sox2, and Myc (OKSM), and an EGFP reporter incorporated into the endogenous Oct4 locus (Oct4-IRES-EGFP). We plated MEFs in serum-containing induction medium, with Dox added on day 0 to induce the OKSM cassette (Phase-1(Dox)). Following Dox withdrawal at day 8, we transferred cells to either serum-free N2B27 2i medium (Phase-2(2i)) or maintained the cells in serum (Phase-2(serum)). Oct4-EGFP+ cells emerged on day 10 as a reporter for successful reprogramming to endogenous Oct4 expression (FIGS. 24A, 30G).

We performed two dense time-course experiments. In the first we collected ˜65,000 scRNA-seq profiles at 10 time points across 16 days, with samples taken every 48 hours. In the second we profiled ˜250,000 cells collected at 39 time points across 18 days, with samples taken every 12 hours (and every 6 hours between days 8 and 9) (FIG. 24A, Methods, Table 14). The density allows us to ensure that the model is fit on a smoothly progressing process, as well as to use some time points as test data for predictions (below). We also collected samples from established iPSC lines reprogrammed from the same MEFs, maintained in either 2i or serum conditions. The two experiments were consistent (STAR Methods). We focused on the second experiment, where we profiled 259,155 cells to an average depth of 46,523 reads per cell (Table 14). After discarding cells with less than 2,000 transcripts detected, we retained a total of 251,203 cells, with a median of 2,565 genes and 9,132 unique transcripts detected per cell.

TABLE 14
Summary of single cell sequencing statistics and sample information.

| | | | | | | Reads | Reads | Reads | Reads |
|---|---|---|---|---|---|---|---|---|---|
| | | Mean | Median | | | Mapped | Mapped | Mapped | Mapped |
| | Estimated | Reads | Genes | Number | | Confidently | Confidently | Confidently | Confidently |
| | Number of | per | per | of | Valid | to | to Exonic | to Intronic | to Intergenic |
| Sample Name | Cells | Cell | Cell | Reads | Barcodes | Transcriptome | Regions | Regions | Regions |
| D0_Dox_C1 | 3495 | 17263 | 2308 | 60336236 | 98 | 62.7 | 66.1 | 10.8 | 5.4 |
| D0_Dox_C2 | 1125 | 41979 | 3559 | 47227004 | 98 | 64.2 | 67.6 | 10.5 | 4.9 |
| D0.5_Dox_C1 | 1220 | 65642 | 4258 | 80083266 | 97.9 | 63.4 | 66.9 | 11.3 | 5 |
| D0.5_Dox_C2 | 2229 | 32317 | 3230 | 72036482 | 98.3 | 61.9 | 65.7 | 10.2 | 5.2 |
| D1_Dox_C1 | 1403 | 12500 | 2366 | 17538332 | 98.1 | 67.8 | 73.6 | 9.7 | 2.9 |
| D1_Dox_C2 | 2332 | 21111 | 2776 | 49231019 | 98.1 | 51.8 | 55.8 | 11.4 | 7.4 |
| D1.5_Dox_C1 | 1639 | 103491 | 4926 | 1.7E+08 | 97.9 | 47.4 | 50.2 | 12.6 | 9.2 |
| D1.5_Dox_C2 | 317 | 253704 | 6159 | 80424447 | 98.3 | 71.1 | 74.9 | 8.9 | 3.1 |
| D2_Dox_C1 | 4360 | 37710 | 3154 | 1.64E+08 | 97.9 | 45.3 | 47.6 | 12.4 | 9.8 |
| D2_Dox_C2 | 5310 | 4443 | 1007 | 23593131 | 98.2 | 71.9 | 75.6 | 7.9 | 3.3 |
| D2.5_Dox_C1 | 3184 | 11931 | 1838 | 37988832 | 98.4 | 57.5 | 60.4 | 10.7 | 5.8 |
| D2.5_Dox_C2 | 3732 | 15914 | 2296 | 59391343 | 98.3 | 65.4 | 69 | 9.4 | 4.4 |
| D3_Dox_C1 | 3673 | 16055 | 2314 | 58972209 | 98.2 | 69.8 | 73.7 | 9.5 | 3.3 |
| D3_Dox_C2 | 3148 | 41424 | 3630 | 1.3E+08 | 98.2 | 68.1 | 71.9 | 9.1 | 3.8 |
| D3.5_Dox_C1 | 4626 | 11906 | 1782 | 55079302 | 98.3 | 70.7 | 74.5 | 9 | 3.3 |
| D3.5_Dox_C2 | 3440 | 6320 | 1284 | 21741409 | 98.3 | 72.4 | 76.3 | 9 | 3 |
| D4_Dox_C1 | 4085 | 23014 | 2532 | 94013331 | 98.4 | 72.3 | 76.1 | 9 | 3 |
| D4_Dox_C2 | 4877 | 34713 | 3078 | 1.69E+08 | 98.1 | 74 | 77.8 | 8.4 | 2.6 |
| D4.5_Dox_C1 | 3551 | 52881 | 3490 | 1.88E+08 | 98.3 | 71.8 | 75.8 | 8.9 | 2.8 |
| D4.5_Dox_C2 | 3576 | 49701 | 3460 | 1.78E+08 | 98.3 | 69.6 | 74.6 | 7.6 | 2.7 |
| D5_Dox_C1 | 4018 | 49996 | 3308 | 2.01E+08 | 98.4 | 69.7 | 74.7 | 7.3 | 2.7 |
| D5_Dox_C2 | 3209 | 77855 | 3986 | 2.5E+08 | 98.3 | 71.7 | 76.5 | 7.4 | 2.5 |
| D5.5_Dox_C1 | 3338 | 44353 | 3032 | 1.48E+08 | 98.4 | 69.7 | 74.5 | 8 | 2.8 |
| D5.5_Dox_C2 | 3212 | 28798 | 2586 | 92501384 | 98.4 | 71.4 | 75.8 | 7.5 | 2.7 |
| D6_Dox_C1 | 5554 | 75461 | 3223 | 4.19E+08 | 98.4 | 73 | 75.5 | 10 | 3.1 |
| D6_Dox_C2 | 2868 | 471033 | 4897 | 1.35E+09 | 98.5 | 71.2 | 73.7 | 9.7 | 3.5 |
| D6.5_Dox_C1 | 535 | 290563 | 4717 | 1.55E+08 | 98.4 | 70.2 | 73.3 | 11.6 | 2.8 |
| D6.5_Dox_C2 | 2576 | 85899 | 4114 | 2.21E+08 | 98.4 | 74.4 | 77.1 | 9.1 | 2.5 |
| D7_Dox_C1 | 3138 | 137190 | 4327 | 4.31E+08 | 98.3 | 70.2 | 73.1 | 11.2 | 3.2 |
| D7_Dox_C2 | 3369 | 80817 | 4154 | 2.72E+08 | 98.3 | 71.1 | 73.9 | 10.7 | 3 |
| D7.5_Dox_C1 | 2591 | 68735 | 3667 | 1.78E+08 | 98.4 | 70.9 | 73.7 | 11.1 | 3.1 |
| D7.5_Dox_C2 | 2470 | 26535 | 2494 | 65541812 | 98.4 | 69.8 | 72.3 | 10 | 3.7 |
| D8_Dox_C1 | 1879 | 17805 | 1644 | 33456383 | 98.2 | 61.3 | 64.3 | 10.4 | 5.7 |
| D8_Dox_C2 | 2139 | 11221 | 1374 | 24003361 | 98.4 | 68.2 | 71.4 | 9.1 | 4.2 |
| D8.25_2i_C1 | 1856 | 15122 | 1692 | 28066499 | 98.3 | 71.5 | 75.2 | 9.2 | 3.3 |
| D8.25_2i_C2 | 2120 | 12979 | 1587 | 27516277 | 98.3 | 67.8 | 71.4 | 9.3 | 4.1 |
| D8.25_serum_C1 | 1549 | 22382 | 1901 | 34670761 | 98.2 | 62.2 | 65 | 10.7 | 5.4 |
| D8.25_serum_C2 | 2379 | 16332 | 1601 | 38854100 | 98.4 | 67.9 | 70.7 | 8.9 | 4.5 |
| D8.5_2i_C1 | 1186 | 60410 | 3119 | 71646422 | 98.2 | 76.5 | 79.6 | 7.2 | 2.4 |
| D8.5_2i_C2 | 1641 | 35193 | 2534 | 57753221 | 98 | 76.6 | 79.8 | 7 | 2.4 |
| D8.5_serum_C1 | 1654 | 40214 | 2653 | 66514572 | 98 | 75.6 | 78.9 | 7.8 | 2.3 |
| D8.5_serum_C2 | 1919 | 31754 | 2451 | 60937426 | 97.9 | 75.6 | 78.6 | 7.7 | 2.4 |
| D8.75_2i_C1 | 1796 | 9830 | 1333 | 17654865 | 98.4 | 72.5 | 75.3 | 9 | 3.2 |
| D8.75_2i_C2 | 1650 | 12257 | 1552 | 20225030 | 98.4 | 73.5 | 76.8 | 8.8 | 2.9 |
| D8.75_serum_C1 | 1616 | 12766 | 1529 | 20630020 | 98.3 | 72.7 | 76 | 9.4 | 2.9 |
| D8.75_serum_C2 | 1526 | 26367 | 2275 | 40237550 | 98.3 | 71.9 | 75 | 9.5 | 3.1 |
| D9_2i_C1 | 1090 | 59016 | 2817 | 64328422 | 97.8 | 76.4 | 79.5 | 7.3 | 2.3 |
| D9_2i_C2 | 944 | 36684 | 2753 | 34630027 | 98.1 | 77.5 | 80.3 | 7 | 2.2 |
| D9_serum_C1 | 1842 | 18322 | 1977 | 33750278 | 98.5 | 83.2 | 85.3 | 4.4 | 1.8 |
| D9_serum_C2 | 1237 | 32382 | 2317 | 40057020 | 98.5 | 81.7 | 83.8 | 5.2 | 2 |
| D9.5_2i_C1 | 991 | 29973 | 2185 | 29703571 | 98.3 | 73.1 | 75.9 | 9.7 | 3.3 |
| D9.5_2i_C2 | 598 | 52831 | 2732 | 31593148 | 98.2 | 70 | 72.9 | 9.6 | 4 |
| D9.5_serum_C1 | 1156 | 27622 | 2056 | 31931324 | 98.2 | 68.6 | 71.4 | 10.9 | 3.9 |
| D9.5_serum_C2 | 1141 | 26127 | 1892 | 29811637 | 98.3 | 75.3 | 78.1 | 8.7 | 2.9 |
| D10_2i_C1 | 1049 | 16523 | 1645 | 17333643 | 98.1 | 61.3 | 63.8 | 12 | 5.9 |
| D10_2i_C2 | 915 | 30277 | 2358 | 27704152 | 98.2 | 64.7 | 67.1 | 11.8 | 5 |
| D10_serum_C1 | 1291 | 26013 | 2068 | 33583765 | 98.1 | 66.7 | 69.3 | 12.7 | 4.1 |
| D10_serum_C2 | 1128 | 7939 | 1210 | 8955917 | 98.3 | 71.1 | 73.6 | 11.9 | 3.3 |
| D10.5_2i_C1 | 767 | 31973 | 2717 | 24523951 | 98.1 | 68.5 | 71.4 | 13 | 3.6 |
| D10.5_2i_C2 | 694 | 25324 | 2369 | 17574924 | 98.1 | 68.8 | 71.5 | 11.9 | 3.6 |
| D10.5_serum_C1 | 964 | 27167 | 32313 | 26189701 | 98.2 | 72 | 74.7 | 11.8 | 2.8 |
| D10.5_serum_C2 | 1022 | 21765 | 2171 | 22243909 | 98.2 | 73.6 | 76 | 11 | 2.7 |
| D11_2i_C1 | 752 | 23981 | 2171 | 18033999 | 98.2 | 75.6 | 78.3 | 9.2 | 2.4 |
| D11_2i_C2 | 603 | 22188 | 2308 | 13379426 | 98.2 | 71.9 | 74.4 | 10.5 | 3 |
| D11_serum_C1 | 1407 | 9160 | 1585 | 12888357 | 98.3 | 75.7 | 78.3 | 10.7 | 2.3 |
| D11_serum_C2 | 1205 | 10612 | 1692 | 12788655 | 98.4 | 78.8 | 81.5 | 8.5 | 2 |
| D11.5_2i_C1 | 720 | 38658 | 2783 | 27834347 | 98.3 | 73.9 | 76.6 | 10.7 | 2.7 |
| D11.5_2i_C2 | 659 | 54360 | 3298 | 35823619 | 98.3 | 74.1 | 76.7 | 10.5 | 2.7 |
| D11.5_serum_C1 | 1178 | 77058 | 3586 | 90774725 | 98.2 | 74.1 | 76.7 | 11.6 | 2.5 |
| D11.5_serum_C2 | 1064 | 14238 | 1903 | 15149367 | 98.2 | 74.9 | 77.4 | 10.9 | 2.4 |
| D12_2i_C1 | 818 | 42704 | 2523 | 34932625 | 98.5 | 74.3 | 77.1 | 8.6 | 2.8 |
| D12_2i_C2 | 621 | 58092 | 2880 | 36075300 | 98.5 | 76 | 78.7 | 7.8 | 2.7 |
| D12_serum_C1 | 1107 | 25116 | 2468 | 27804384 | 98.4 | 76.1 | 78.7 | 9.4 | 2.4 |
| D12_serum_C2 | 1322 | 20552 | 2358 | 27170840 | 98.4 | 76.4 | 79.2 | 9.3 | 2.3 |
| D12.5_2i_C1 | 689 | 32471 | 2560 | 22372820 | 98.4 | 73.7 | 76.8 | 8.5 | 2.9 |
| D12.5_2i_C2 | 668 | 54768 | 3214 | 36585438 | 98.4 | 73.8 | 76.8 | 8.4 | 2.9 |
| D12.5_serum_C1 | 1052 | 29456 | 2816 | 30987716 | 98.3 | 76.8 | 79.7 | 8.5 | 2.3 |
| D12.5_serum_C2 | 1201 | 138451 | 4369 | 1.66E+08 | 98.3 | 76.3 | 79.2 | 8.8 | 2.4 |
| D13_2i_C1 | 655 | 75220 | 2938 | 49269432 | 98.3 | 72.1 | 75.5 | 8.8 | 3.1 |
| D13_2i_C2 | 643 | 156892 | 2866 | 1.01E+08 | 98.3 | 73.4 | 76.8 | 8.3 | 2.8 |
| D13_serum_C1 | 980 | 99956 | 3179 | 97956936 | 98.3 | 75 | 78.1 | 9.6 | 2.4 |
| D13_serum_C2 | 1166 | 93789 | 3646 | 1.09E+08 | 98.3 | 73.8 | 77 | 10.3 | 2.5 |
| D13.5_2i_C1 | 1054 | 46666 | 1996 | 49186630 | 97.5 | 60.7 | 65.4 | 16 | 4.9 |
| D13.5_2i_C2 | 827 | 26735 | 1853 | 22110011 | 97.5 | 59 | 63.3 | 15.7 | 5.4 |
| D13.5_serum_C1 | 1268 | 43074 | 2056 | 54618691 | 97.3 | 65.9 | 70.3 | 14.9 | 3.4 |
| D13.5_serum_C2 | 1105 | 42121 | 2126 | 46544722 | 97.3 | 66.3 | 70.6 | 14.6 | 3.5 |
| D14_2i_C1 | 1898 | 39097 | 3022 | 74206890 | 98.3 | 73.3 | 77.5 | 7.6 | 3.1 |
| D14_2i_C2 | 1938 | 54136 | 3577 | 1.05E+08 | 98.4 | 73.5 | 77.6 | 7.4 | 3.1 |
| D14_serum_C1 | 2032 | 34487 | 2897 | 70077873 | 98.3 | 73.7 | 77.2 | 11.2 | 2.5 |
| D14_serum_C2 | 1726 | 56705 | 3539 | 97873582 | 98.3 | 74.3 | 77.6 | 10.4 | 2.6 |
| D14.5_2i_C1 | 2037 | 39164 | 2744 | 79779089 | 98.3 | 69.7 | 74.4 | 9.3 | 3.4 |
| D14.5_2i_C2 | 2089 | 37795 | 3074 | 78954514 | 98.3 | 71 | 75.4 | 8.7 | 3.3 |
| D14.5_serum_C1 | 1346 | 33892 | 2505 | 45618882 | 98.2 | 71.6 | 75.8 | 12 | 2.7 |
| D14.5_serum_C2 | 1377 | 76526 | 3705 | 1.05E+08 | 98.4 | 75.6 | 78.9 | 10 | 2.4 |
| D15_2i_C1 | 2558 | 32100 | 1935 | 82113379 | 97.4 | 56.2 | 63.1 | 18 | 5 |
| D15_2i_C2 | 2279 | 20244 | 2111 | 46137688 | 97.9 | 62.2 | 67.5 | 14.1 | 4.8 |
| D15_serum_C1 | 1766 | 48958 | 3162 | 86460491 | 98.3 | 75.7 | 79 | 10 | 2.3 |
| D15_serum_C2 | 2157 | 25885 | 2007 | 55835189 | 97.8 | 69.5 | 74 | 13.5 | 2.9 |
| D15.5_2i_C1 | 4277 | 16535 | 1964 | 70721479 | 98.2 | 72.7 | 76.8 | 7.7 | 3.4 |
| D15.5_2i_C2 | 3402 | 19528 | 2143 | 66435427 | 98.3 | 73 | 76.8 | 7.6 | 3.4 |
| D15.5_serum_C1 | 2295 | 107956 | 3685 | 2.48E+08 | 98.2 | 70.8 | 74.5 | 12.6 | 2.9 |
| D15.5_serum_C2 | 2556 | 64367 | 3347 | 1.65E+08 | 98.2 | 70.4 | 74.2 | 12.5 | 3 |
| D16_2i_C1 | 3927 | 13315 | 1343 | 52290532 | 98.4 | 72.9 | 76.2 | 8.1 | 3.6 |
| D16_2i_C2 | 2800 | 18996 | 1921 | 53190608 | 98.4 | 73.4 | 76.8 | 7.8 | 3.4 |
| D16_serum_C1 | 1749 | 27763 | 2182 | 48558555 | 98.1 | 75 | 78.3 | 8.7 | 2.5 |
| D16_serum_C2 | 1693 | 28886 | 2467 | 48904299 | 98.2 | 73.7 | 77.3 | 10.4 | 2.6 |
| D16.5_2i_C1 | 3204 | 17424 | 2124 | 55829324 | 98.3 | 74 | 77.6 | 7.5 | 3.3 |
| D16.5_2i_C2 | 4094 | 10237 | 1618 | 41911584 | 98.3 | 73.9 | 77.4 | 7.3 | 3.3 |
| D16.5_serum_C1 | 2350 | 57651 | 3393 | 1.35E+08 | 98.2 | 72.6 | 75.9 | 11.7 | 2.8 |
| D16.5_serum_C2 | 2310 | 22716 | 2119 | 52474229 | 98.2 | 73.9 | 77.1 | 10.1 | 2.7 |
| D17_2i_C1 | 2321 | 28918 | 2807 | 67119554 | 98.3 | 73.9 | 77.2 | 7.8 | 3.4 |
| D17_2i_C2 | 2111 | 22044 | 2539 | 46535861 | 98.4 | 74.7 | 77.9 | 7.5 | 3.3 |
| D17_serum_C1 | 1561 | 62052 | 3583 | 96863752 | 98.3 | 71.9 | 75.1 | 11.5 | 3 |
| D17_serum_C2 | 2117 | 45803 | 3300 | 96965300 | 98.3 | 71.6 | 75 | 11.5 | 3 |
| D17.5_2i_C1 | 1638 | 36580 | 2900 | 59918421 | 98.5 | 75.4 | 78.6 | 6.9 | 3.2 |
| D17.5_2i_C2 | 2413 | 22428 | 2474 | 54120470 | 98.4 | 75.4 | 78.7 | 6.9 | 3.1 |
| D17.5_serum_C1 | 1957 | 44221 | 3292 | 86540688 | 98.4 | 73.1 | 76.4 | 10.3 | 2.9 |
| D17.5_serum_C2 | 2112 | 29527 | 2849 | 62361742 | 98.4 | 74.6 | 77.7 | 10.1 | 2.7 |
| D18_2i_C1 | 1989 | 69937 | 2774 | 1.39E+08 | 98.4 | 74.3 | 77.5 | 6.3 | 3.5 |
| D18_2i_C2 | 1648 | 63038 | 2761 | 1.04E+08 | 98.4 | 75 | 78.2 | 6 | 3.4 |
| D18_serum_C1 | 1898 | 62257 | 2472 | 1.18E+08 | 98.3 | 72.1 | 75.5 | 10.4 | 3 |
| D18_serum_C2 | 1902 | 40600 | 2322 | 77222647 | 98.3 | 73.6 | 76.8 | 9.3 | 2.8 |
| DiPSC_2i_C1 | 3466 | 21467 | 2524 | 74406713 | 98.2 | 67.7 | 71.6 | 9.7 | 3.8 |
| DiPSC_2i_C2 | 1872 | 46879 | 3649 | 87759016 | 98.3 | 67.6 | 71.7 | 9.5 | 3.8 |
| DiPSC_serum_C1 | 5247 | 18112 | 2241 | 95034273 | 98.2 | 65.9 | 70.1 | 10.3 | 4.4 |
| DiPSC_serum_C2 | 4340 | 21502 | 2535 | 93322919 | 98.2 | 67.5 | 71.4 | 9.8 | 4 |

| | | | | Q30 | | | | | |
|---|---|---|---|---|---|---|---|---|---|
| | Reads | | Q30 | Bases | Q30 | Q30 | | | Median |
| | Mapped | | Bases | in | Bases | Bases | Fraction | Total | UMI |
| | Antisense | Sequencing | in | RNA | in Sample | in | Reads in | Genes | Counts |
| Sample Name | to Gene | Saturation | Barcode | Read | Index | UMI | Cells | Detected | per Cell |
| D0_Dox_C1 | 4.4 | 17.4 | 97.9 | 90.9 | 95.8 | 97.7 | 92.2 | 16467 | 7421 |
| D0_Dox_C2 | 4.3 | 30.8 | 97.9 | 90.6 | 96.3 | 97.7 | 92.4 | 15884 | 15756 |
| D0.5_Dox_C1 | 4.4 | 38.7 | 97.9 | 90.6 | 95.8 | 97.7 | 95.5 | 16658 | 22429 |
| D0.5_Dox_C2 | 4.6 | 22.5 | 97.8 | 87.8 | 96.2 | 97.5 | 90.3 | 16911 | 12851 |
| D1_Dox_C1 | 6.6 | 12.8 | 97.7 | 85.3 | 95.8 | 97.4 | 89 | 15028 | 6263 |
| D1_Dox_C2 | 5.2 | 13.5 | 97.8 | 88.2 | 96 | 97.5 | 94 | 16161 | 8318 |
| D1.5_Dox_C1 | 4 | 33.3 | 97.9 | 91.3 | 95.5 | 97.7 | 91.8 | 17182 | 27357 |
| D1.5_Dox_C2 | 4.7 | 64.6 | 97.9 | 89 | 96.1 | 97.6 | 78.5 | 15562 | 48498 |
| D2_Dox_C1 | 3.5 | 18.9 | 97.9 | 90.5 | 96.1 | 97.6 | 92.5 | 17003 | 11247 |
| D2_Dox_C2 | 4.4 | 10.2 | 97.8 | 88.8 | 95.9 | 97.6 | 87.1 | 14980 | 2275 |
| D2.5_Dox_C1 | 3.9 | 13 | 98 | 90.6 | 96.3 | 97.8 | 92.7 | 15423 | 5041 |
| D2.5_Dox_C2 | 4.2 | 14.7 | 97.8 | 87.4 | 95.6 | 97.5 | 95.6 | 16143 | 7728 |
| D3_Dox_C1 | 4.4 | 15.8 | 97.8 | 87.6 | 95.9 | 97.5 | 94.4 | 16144 | 8215 |
| D3_Dox_C2 | 4.2 | 26.1 | 97.7 | 87.1 | 96.1 | 97.5 | 93.5 | 17099 | 18216 |
| D3.5_Dox_C1 | 4.6 | 15.3 | 97.9 | 89.3 | 95.7 | 97.6 | 96.3 | 15929 | 6318 |
| D3.5_Dox_C2 | 4.6 | 12.1 | 97.9 | 89.7 | 96.3 | 97.6 | 96.6 | 14788 | 3562 |
| D4_Dox_C1 | 4.5 | 22.5 | 97.9 | 89.6 | 96.1 | 97.6 | 97 | 16574 | 11428 |
| D4_Dox_C2 | 4.5 | 28.9 | 97.9 | 89.7 | 95.9 | 97.6 | 97.6 | 17265 | 16183 |
| D4.5_Dox_C1 | 4.7 | 38.2 | 97.8 | 87.9 | 96 | 97.6 | 95.9 | 17466 | 20437 |
| D4.5_Dox_C2 | 5.5 | 31.5 | 97.6 | 83.1 | 95.3 | 97.3 | 96.2 | 17681 | 20725 |
| D5_Dox_C1 | 5.5 | 34.4 | 97.6 | 82.9 | 95.7 | 97.3 | 96.3 | 17882 | 20293 |
| D5_Dox_C2 | 5.1 | 42.1 | 97.5 | 84.1 | 95.2 | 97 | 94.9 | 17837 | 28005 |
| D5.5_Dox_C1 | 5.4 | 37.5 | 97.6 | 83.4 | 95.3 | 97.3 | 96 | 17425 | 16917 |
| D5.5_Dox_C2 | 5 | 27.4 | 97.6 | 84.3 | 95.9 | 97.3 | 96 | 16996 | 12974 |
| D6_Dox_C1 | 3.7 | 56.6 | 98 | 92 | 96 | 97.8 | 95.1 | 18190 | 19034 |
| D6_Dox_C2 | 4 | 85.2 | 98.1 | 93.2 | 96.4 | 97.9 | 95.6 | 18938 | 39404 |
| D6.5_Dox_C1 | 4.5 | 81.8 | 98 | 92.6 | 96.4 | 97.8 | 96.7 | 16277 | 32776 |
| D6.5_Dox_C2 | 3.9 | 54.1 | 98 | 92.1 | 96 | 97.8 | 96.2 | 17548 | 25293 |
| D7_Dox_C1 | 4.1 | 65.5 | 98 | 92.1 | 96.2 | 97.8 | 94.8 | 18209 | 27686 |
| D7_Dox_C2 | 4 | 47.9 | 98 | 92.2 | 96 | 97.8 | 95.5 | 18024 | 25478 |
| D7.5_Dox_C1 | 3.9 | 51.1 | 98 | 92 | 96 | 97.8 | 94.3 | 17416 | 19859 |
| D7.5_Dox_C2 | 3.8 | 26.3 | 98 | 92.3 | 95.7 | 97.8 | 92.7 | 16519 | 11274 |
| D8_Dox_C1 | 3.9 | 23.2 | 97.9 | 90.9 | 95.8 | 97.6 | 90.6 | 15616 | 6435 |
| D8_Dox_C2 | 3.9 | 20.7 | 97.9 | 90.4 | 96.1 | 97.6 | 91.7 | 15285 | 4995 |
| D8.25_2i_C1 | 4.4 | 21.2 | 97.9 | 90.3 | 96 | 97.6 | 93.1 | 15657 | 6758 |
| D8.25_2i_C2 | 4.5 | 19.1 | 97.9 | 90.3 | 96 | 97.6 | 92.6 | 15714 | 5702 |
| D8.25_serum_C1 | 3.8 | 25.9 | 97.9 | 91.4 | 95.6 | 97.7 | 90.7 | 15808 | 7892 |
| D8.25_serum_C2 | 3.6 | 25.2 | 97.9 | 90.7 | 96.1 | 97.7 | 88.9 | 15972 | 6359 |
| D8.5_2i_C1 | 3.8 | 50.1 | 98 | 93.5 | 96.3 | 97.8 | 92.6 | 16274 | 19378 |
| D8.5_2i_C2 | 3.9 | 36.2 | 98 | 93.5 | 96.2 | 97.8 | 92.8 | 16219 | 14092 |
| D8.5_serum_C1 | 4 | 39.6 | 98 | 93.4 | 95.7 | 97.8 | 90.7 | 16335 | 14336 |
| D8.5_serum_C2 | 3.9 | 35.8 | 98 | 93.6 | 96 | 97.8 | 91.9 | 16274 | 12381 |
| D8.75_2i_C1 | 3.7 | 17.6 | 98 | 91.7 | 96.1 | 97.7 | 92.2 | 15033 | 4785 |
| D8.75_2i_C2 | 3.9 | 19.1 | 97.9 | 90.5 | 95.7 | 97.7 | 92.2 | 15231 | 5962 |
| D8.75_serum_C1 | 3.9 | 18.8 | 97.9 | 90.1 | 95.8 | 97.6 | 89.6 | 15445 | 5629 |
| D8.75_serum_C2 | 3.7 | 26.3 | 97.9 | 90.6 | 96.1 | 97.7 | 87.1 | 16266 | 10133 |
| D9_2i_C1 | 3.9 | 52.1 | 98 | 93.7 | 96.4 | 97.8 | 85.3 | 16091 | 15871 |
| D9_2i_C2 | 3.6 | 42.9 | 98 | 93.7 | 96.2 | 97.8 | 94.5 | 15694 | 13794 |
| D9_serum_C1 | 3 | 52.1 | 98 | 93.5 | 96.2 | 97.8 | 95 | 15502 | 6160 |
| D9_serum_C2 | 3.1 | 64.2 | 98 | 93.6 | 96 | 97.9 | 95.2 | 15526 | 8071 |
| D9.5_2i_C1 | 3.3 | 40.4 | 97.9 | 90.4 | 95.9 | 97.6 | 90.5 | 15662 | 9665 |
| D9.5_2i_C2 | 3.5 | 49.8 | 97.9 | 90.7 | 96.3 | 97.7 | 89.9 | 15572 | 13737 |
| D9.5_serum_C1 | 3.5 | 39.1 | 97.9 | 90.8 | 96.1 | 97.7 | 87.2 | 15936 | 8356 |
| D9.5_serum_C2 | 3.2 | 41.1 | 97.9 | 90.3 | 96.2 | 97.6 | 86.6 | 15754 | 8383 |
| D10_2i_C1 | 3.5 | 24.7 | 98 | 92.5 | 95.9 | 97.8 | 91.3 | 15323 | 5660 |
| D10_2i_C2 | 3.5 | 33.7 | 98 | 92.3 | 95.9 | 97.8 | 92.5 | 15798 | 9422 |
| D10_serum_C1 | 3.6 | 31.1 | 98 | 92.2 | 96 | 97.8 | 83.5 | 16178 | 7906 |
| D10_serum_C2 | 3.4 | 15.8 | 98 | 91.9 | 95.6 | 97.8 | 85.1 | 14888 | 3321 |
| D10.5_2i_C1 | 3.7 | 30.1 | 98 | 91.8 | 95.5 | 97.7 | 92.4 | 16115 | 11465 |
| D10.5_2i_C2 | 3.7 | 25.8 | 98 | 91.9 | 95.7 | 97.7 | 91.8 | 15697 | 9225 |
| D10.5_serum_C1 | 3.8 | 29 | 98 | 91.7 | 96 | 97.8 | 72.5 | 15951 | 8158 |
| D10.5_serum_C2 | 3.5 | 30.8 | 98 | 92.2 | 96.1 | 97.8 | 78.8 | 15650 | 6896 |
| D11_2i_C1 | 3.7 | 29.4 | 98 | 92 | 96.2 | 97.8 | 79.2 | 15758 | 8173 |
| D11_2i_C2 | 3.8 | 27.2 | 98 | 92.6 | 95.7 | 97.8 | 89.8 | 15560 | 8421 |
| D11_serum_C1 | 3.5 | 19.4 | 98 | 91.5 | 96.1 | 97.8 | 86 | 15335 | 4054 |
| D11_serum_C2 | 3.6 | 25.6 | 97.9 | 90.4 | 95.7 | 97.7 | 80.8 | 15379 | 4176 |
| D11.5_2i_C1 | 3.7 | 40.9 | 98 | 92 | 95.5 | 97.8 | 88.4 | 16398 | 11511 |
| D11.5_2i_C2 | 3.63 | 49 | 97.9 | 91.9 | 96.3 | 97.7 | 90.7 | 16538 | 14816 |
| D11.5_serum_C1 | 3.5 | 60.1 | 98 | 91.6 | 96.2 | 97.8 | 85.8 | 17172 | 15611 |
| D11.5_serum_C2 | 3.5 | 23.6 | 98 | 91.9 | 95.6 | 97.8 | 86.2 | 15665 | 5562 |
| D12_2i_C1 | 4.1 | 51.4 | 98 | 92 | 96.2 | 97.8 | 86.2 | 16604 | 10044 |
| D12_2i_C2 | 3.8 | 55.3 | 98 | 91.4 | 96 | 97.8 | 85 | 16529 | 12519 |
| D12_serum_C1 | 3.6 | 35.4 | 98 | 91 | 96 | 97.7 | 84.8 | 16471 | 8119 |
| D12_serum_C2 | 3.6 | 29.9 | 97.9 | 90.6 | 96.2 | 97.7 | 85.4 | 16513 | 7210 |
| D12.5_2i_C1 | 4.1 | 37.9 | 97.9 | 91 | 96.1 | 97.7 | 84.3 | 16343 | 10070 |
| D12.5_2i_C2 | 4 | 47.7 | 97.9 | 91.2 | 96.1 | 97.7 | 86 | 16879 | 15004 |
| D12.5_serum_C1 | 3.7 | 35 | 97.9 | 90.8 | 96 | 97.7 | 84.7 | 16850 | 10108 |
| D12.5_serum_C2 | 3.8 | 67.1 | 97.9 | 90.8 | 96.1 | 97.7 | 81.5 | 18479 | 21756 |
| D13_2i_C1 | 4.3 | 56.4 | 98 | 90.8 | 96.1 | 97.7 | 66.3 | 16853 | 12776 |
| D13_2i_C2 | 4.3 | 72.9 | 98 | 90.8 | 95.8 | 97.7 | 49.1 | 16820 | 11522 |
| D13_serum_C1 | 4 | 73.7 | 98 | 92.1 | 96.3 | 97.8 | 77.6 | 17377 | 12190 |
| D13_serum_C2 | 4 | 67.1 | 98 | 92.2 | 96.1 | 97.8 | 85.4 | 18070 | 15494 |
| D13.5_2i_C1 | 5.7 | 69.4 | 98 | 92.5 | 96.3 | 97.8 | 74.6 | 16769 | 5599 |
| D13.5_2i_C2 | 5.3 | 52.4 | 97.9 | 90.8 | 95.7 | 97.7 | 75.3 | 15987 | 5146 |
| D13.5_serum_C1 | 5.6 | 70.2 | 98 | 90.9 | 95.9 | 97.8 | 77.2 | 16853 | 5287 |
| D13.5_serum_C2 | 5.5 | 68.1 | 97.9 | 91 | 95.9 | 97.8 | 71.1 | 16725 | 5360 |
| D14_2i_C1 | 4.9 | 37 | 98 | 91.8 | 96.3 | 97.8 | 91.6 | 18525 | 15207 |
| D14_2i_C2 | 4.8 | 42.1 | 97.9 | 91.7 | 96.2 | 97.7 | 93.6 | 18764 | 20543 |
| D14_serum_C1 | 4.1 | 39.5 | 97.9 | 91.4 | 96 | 97.7 | 87.9 | 18461 | 10816 |
| D14_serum_C2 | 3.9 | 50.7 | 98 | 91.5 | 96.1 | 97.7 | 87.1 | 18884 | 14705 |
| D14.5_2i_C1 | 5.6 | 36.7 | 98 | 92 | 96 | 97.8 | 81.5 | 18532 | 12798 |
| D14.5_2i_C2 | 5.3 | 33.7 | 98 | 92 | 95.6 | 97.8 | 89.7 | 18770 | 15068 |
| D14.5_serum_C1 | 4.9 | 42 | 98 | 91.6 | 96.1 | 97.8 | 78.9 | 18018 | 8409 |
| D14.5_serum_C2 | 4.1 | 59.7 | 98 | 91.9 | 96.4 | 97.8 | 79.2 | 18580 | 14650 |
| D15_2i_C1 | 7.9 | 61.6 | 98 | 91.6 | 96.2 | 97.8 | 85.3 | 18159 | 5664 |
| D15_2i_C2 | 6 | 38.4 | 97.9 | 91.5 | 95.7 | 97.7 | 92.1 | 17960 | 7023 |
| D15_serum_C1 | 3.9 | 39.9 | 98 | 91.5 | 95.7 | 97.8 | 66.9 | 18739 | 11915 |
| D15_serum_C2 | 5.1 | 46 | 98 | 91.6 | 96 | 97.8 | 63.9 | 18103 | 5252 |
| D15.5_2i_C1 | 4.5 | 21.3 | 97.9 | 91.6 | 96 | 97.7 | 94.4 | 18490 | 8467 |
| D15.5_2i_C2 | 4.3 | 23 | 97.9 | 92.1 | 96.3 | 97.7 | 94.3 | 18358 | 9841 |
| D15.5_serum_C1 | 4.3 | 66.5 | 98 | 92 | 95.9 | 97.8 | 76.9 | 19807 | 15905 |
| D15.5_serum_C2 | 4.4 | 54.1 | 98 | 91.9 | 96 | 97.8 | 82.2 | 19970 | 13986 |
| D16_2i_C1 | 3.7 | 38.5 | 98 | 91.9 | 96.3 | 97.8 | 92.2 | 17665 | 5076 |
| D16_2i_C2 | 3.7 | 25.7 | 97.9 | 91.8 | 96.2 | 97.7 | 94.5 | 17761 | 9135 |
| D16_serum_C1 | 4 | 30.4 | 97.9 | 91.5 | 95.6 | 97.8 | 57 | 18278 | 6791 |
| D16_serum_C2 | 4.1 | 36.6 | 97.9 | 91.3 | 96.1 | 97.7 | 78.1 | 18336 | 8342 |
| D16.5_2i_C1 | 4.2 | 22.6 | 97.9 | 91.8 | 96.3 | 97.8 | 89.2 | 18679 | 8471 |
| D16.5_2i_C2 | 4.2 | 15.9 | 97.9 | 91.6 | 96.2 | 97.7 | 88.7 | 18674 | 5373 |
| D16.5_serum_C1 | 3.9 | 47.3 | 98 | 91.5 | 96.1 | 97.8 | 76.4 | 19896 | 13361 |
| D16.5_serum_C2 | 3.9 | 28.2 | 98 | 91.7 | 96.3 | 97.8 | 65.7 | 18796 | 6278 |
| D17_2i_C1 | 3.9 | 29.8 | 98 | 91.9 | 96.2 | 97.8 | 89.9 | 18877 | 12668 |
| D17_2i_C2 | 3.8 | 23.6 | 98 | 91.7 | 96.2 | 97.8 | 90.5 | 18501 | 10936 |
| D17_serum_C1 | 3.9 | 49.4 | 98 | 91.8 | 96.1 | 97.8 | 88.1 | 19538 | 15523 |
| D17_serum_C2 | 4 | 42 | 98 | 91.5 | 96.2 | 97.8 | 86.3 | 19729 | 12979 |
| D17.5_2i_C1 | 3.8 | 40.2 | 98 | 92.1 | 96.3 | 97.8 | 92.1 | 18309 | 14477 |
| D17.5_2i_C2 | 4 | 28.2 | 98 | 91.8 | 95.9 | 97.8 | 92.2 | 18452 | 10753 |
| D17.5_serum_C1 | 4 | 44.1 | 97.9 | 91.4 | 96.3 | 97.8 | 85.1 | 19556 | 12806 |
| D17.5_serum_C2 | 3.8 | 36.5 | 98 | 91.8 | 96 | 97.8 | 87.9 | 19155 | 9998 |
| D18_2i_C1 | 3.9 | 58.2 | 98 | 92.6 | 96.2 | 97.8 | 90.9 | 18821 | 18060 |
| D18_2i_C2 | 3.7 | 54.8 | 98 | 92.5 | 96.3 | 97.8 | 90.6 | 18566 | 17916 |
| D18_serum_C1 | 4.1 | 62.7 | 98 | 92.3 | 96 | 97.8 | 80 | 19294 | 9840 |
| D18_serum_C2 | 3.9 | 48.1 | 98 | 92 | 96.4 | 97.8 | 77.3 | 19023 | 9029 |
| DiPSC_2i_C1 | 5.1 | 20.2 | 98 | 91.3 | 96.1 | 97.7 | 96.4 | 17918 | 10626 |
| DiPSC_2i_C2 | 5.3 | 28.8 | 97.9 | 90.9 | 96.1 | 97.7 | 96.2 | 18049 | 20527 |
| DiPSC_serum_C1 | 5.1 | 23.2 | 97.9 | 90.1 | 95.9 | 97.7 | 93.2 | 19202 | 7777 |
| DiPSC_serum_C2 | 4.9 | 23.3 | 97.9 | 90.9 | 96.1 | 97.7 | 90.8 | 19098 | 9449 |

A Model of the Developmental Landscape

We visualized the developmental landscape of the 251,203 cells in a two-dimensional FLE (FIG. 24B) and annotated it according to sampling time (FIG. 24C), expression scores of gene signatures, and expression of individual genes (FIG. 24D, Table 15).

TABLE 15
List of genes comprising gene signatures.
MEF identity

| | | | | | | | |
|---|---|---|---|---|---|---|---|
| Gm5571 | Il17rd | Gjd4 | Prss23 | Atp10a | Eif4g2 | Gulp1 | Sema3a |
| Rbfox2 | Ptk2 | Ccng1 | 9430030n17rik | Loxl1 | Vcl | Shank1 | Itgb1 |
| Btbd19 | Ehd2 | Gpr124 | Arntl2 | Loxl2 | Bcl2l2 | Bmp1 | Nxn |
| Actn1 | Lats2 | Fibin | Sh3rf1 | Fbln5 | Cd276 | Akt1s1 | Tmem41b |
| Gatad2a | Hspg2 | 8030476l19rik | Mrc2 | Ctgf | Lrrc58 | Itga9 | Sec23a |
| Med6 | 4930456g14rik | Ddr2 | Mdh1 | Efnb2 | Wwc2 | Abcc1 | Gm22 |
| Mex3a | 4930429b21rik | Arf4 | Rictor | Rxra | Lpp | Eda | Itgb5 |
| Ccdc80 | Rps20 | Ptprs | Map4k5 | Ccnd2 | Arl1 | B4galt2 | Dysf |
| Mex3c | Vgll3 | Sprr2k | Plcl1 | Gpc2 | Ltbp1 | Nid1 | Thbs1 |
| Sdpr | Prr15 | Adm | 11-Sep | Ntf3 | Ltbp2 | Ncam1 | Bc022687 |
| Pcdhb2 | Fbxl7 | A830029e22rik | Ryk | Kif5b | Wisp1 | Shc2 | Dnm3os |
| Trim16 | Maged2 | 9230114k14rik | Tgfb3 | Slit2 | Igf1r | Uba6 | Rnd3 |
| Obsl1 | Galntl4 | Extl3 | Ube2i | Tpm1 | Rhobtb3 | Tradd | Pik3c2a |
| Epha1 | Pdgfc | Mecom | Tgfb2 | Gpc4 | Fam198b | Rtel1 | 2810008m24rik |
| Stx1b | Tmtc4 | Qsox1 | Zfp319 | Flnb | Cnn2 | Bicd2 | Spred3 |
| Stau1 | Tmtc3 | Tead1 | Gm10399 | 4930555b11rik | Glipr2 | Adamts12 | Senp5 |
| Serpine1 | Lpar4 | Snx7 | Fbxo17 | Flnc | Syde1 | Hs2st1 | Arl13b |
| Aa881470 | Pcdh19 | Cdkl4 | Wnt5a | C76332 | Hhat | D10ertd610e | Polr2e |
| Col12a1 | Eda2r | Cdkn2a | Crim1 | Capn2 | Zmat3 | Cyr61 | Itgav |
| 2010300f17rik | Pcdh18 | Cdkn2b | Mid1 | Phlda3 | Cald1 | Gtf3c1 | Igf2bp3 |
| Ccdc102a | Gpr176 | Ccnyl1 | Disp1 | Map3k7 | Pmepa1 | Lbh | |
| Nradd | Loc100503471 | Tubb2a-ps2 | Ubox5 | Myh10 | E130112l23rik | Krt33b | |
| Pard6g | Mical2 | Aen | St7l | D18ertd653e | Bag2 | Gm6607 | |
| Nta4 | Dzip1l | Farp1 | Col5a2 | Stox2 | Zfp583 | D3wsu167e | |
| 5730471h19rik | Hoxc6 | 4930402h24rik | Axl | Igf2r | Pibf1 | Zc3h7b | |
| Sepn1 | Hoxc5 | Sh3rf3 | Col5a1 | D15ertd621e | Pmaip1 | 7630403g23rik | |
| Peg12 | Mettl4-ps1 | Adam19 | Zyx | Arid5b | A130022j15rik | Tnpo2 | |
| Dpysl3 | Sec63 | Ddb1 | Ror2 | Tnfrsf10b | Bcl9l | Cep170 | |
| 1110012d08rik | Ikbip | Cttn | Wdfy3 | 2610011e03rik | Cpa6 | Pdlim5 | |
| Akt1 | Tsc22d2 | 9230112e08rik | Amotl2 | Ckap4 | D13ertd787e | Pdlim7 | |
| Zfp286 | 2310076g05rik | Dbn1 | Yap1 | Efna2 | Pabpc4l | Cad | |
| Ubap2l | Anxa6 | Fyttd1 | Phldb2 | Picalm | Zfhx3 | Unc5b | |
| Samd4 | Nfatc4 | Lrrc15 | 6330562c20rik | Cdh10 | Itga5 | 2410018113rik | |
| Phc2 | Fn1 | Fkbp10 | Ctnnd1 | Ddah1 | Txnrd1 | Loc100216343 | |
| Mcam | Wnt9a | Trub1 | Rock2 | Uba3 | Htr1b | Glrx3 | |
| Pla2g4c | Sorcs2 | Zdhhc20 | Masp1 | 0610038b21rik | Hmga2 | Kctd5 | |
| Fzd7 | Tmeff1 | Ston1 | Pvt1 | Gemin7 | 2-Sep | Loc269472 | |
| Pappa | C79491 | Hoxd13 | Tnc | Uba1 | Lamb1 | Myo1c | |
| Ptk7 | Crlf1 | Nudt6 | Fbln2 | Fbn1 | Zfp518b | 4930562c15rik | |
| Nuak1 | 2610034e01rik | Hoxd12 | Hdlbp | Lhx9 | Parva | Tll1 | |

Pluripotency

| | | | | | | | |
|---|---|---|---|---|---|---|---|
| Rhox5 | Mt2 | Asns | Taf7 | Folr1 | Sox2 | Grhpr | Chmp4c |
| Tdgf1 | Ube2a | Aldoa | Nudt4 | Gm7325 | Jam2 | Higd1a | Hsf2bp |
| Utf1 | Khdc3 | Tdh | Cox5a | Agtrap | Fkbp3 | Rpp25 | Polr2e |
| Mkm1 | Pycard | Gjb3 | Sod2 | Spp1 | Cox7b | Rbpms | Blvrb |
| Dppa5a | Hsp90aa1 | Rbpms2 | S100a13 | Hells | Ash2l | Mmp3 | Ldhb |
| Upp1 | Prrc1 | Pips1 | Fkbp6 | Dppa4 | Dut | Apobec3 | Apoc1 |
| Chchd10 | Hat1 | Fam25c | Rhox9 | Gabarapl2 | Dtymk | Spc24 | Syngr1 |
| Klf2 | Calcoco2 | Eif2s2 | Gdf3 | Rhox6 | Gpx4 | Xlr3a | Bex1 |
| Trap1a | Impa2 | Cenpm | 2700094K13Rik | Rhox1 | Eif4ebp1 | Rec114 | Nr2c2ap |
| Mylpf | Saa3 | Nanog | Fmr1nb | Cdc51 | Morc1 | Mtf2 | |
| 1700013H16Rik | Ooep | Ndufa4l2 | Hmgn2 | Tex19.1 | Fabp3 | Snrpn | |
| AA467197 | Bnip3 | Syce2 | Ubald2 | Trim28 | Zfp428 | Gm13580 | |
| Dhx16 | Mt1 | Gm13251 | Lactb2 | Atp5gl | Aqp3 | Gmnn | |

Cell cycle

| | | | | | | | |
|---|---|---|---|---|---|---|---|
| Mcm4 | Lbr | Cdk1 | Ndc80 | Cdca2 | Rrm2 | Hjurp | Rpa2 |
| Smc4 | Cenpf | Slbp | Mcm6 | Nasp | Tipin | Tacc3 | Gins2 |
| Gtse1 | Birc5 | Aurkb | Rrm1 | Gmnn | Casp8ap2 | Mcm5 | E2f8 |
| Ttk | Dtl | Kif1l | Mlf1ip | Cdc6 | Tubb4b | Anp32e | Cdc25c |
| Rangap1 | Dscc1 | Cks1b | Top2a | Pold3 | Kif23 | Dlgap5 | Nek2 |
| Ccnb2 | Cbx5 | Blm | Hmgb2 | Ckap2l | Exo1 | Ect2 | Cdc20 |
| Cenpa | Usp1 | Msh2 | Ccne2 | Fam64a | Rfc2 | Nuf2 | Rad51ap1 |
| Cenpe | Hmmr | Gas2l3 | G2e3 | Ubr7 | Pola1 | Cdc45 | |
| Cdca8 | Wdr76 | Tyms | Tmpo | Fen1 | Mki67 | Ckap5 | |
| Ckap2 | Ung | Hjurp | Nusap1 | Bub1 | Tpx2 | Ctcf | |
| Rad51 | Hn1 | Hells | Ncapd2 | Brip1 | Aurka | Clspn | |
| Pcna | Cks2 | Prim1 | Mcm2 | Atad2 | Anln | Cdca7 | |
| Ube2c | Kif20b | Uhrf1 | Kif2c | Psrc1 | Chaf1b | Cdca3 | |

ER Stress

| | | | | | | | |
|---|---|---|---|---|---|---|---|
| Nck2 | Chac1 | Creb3 | Itpr1 | Os9 | Stt3b | Dnajb9 | Crebrf |
| Ankzf1 | Pdia3 | Sec61b | Edem1 | Ddit3 | Rnf185 | Tmx1 | Bak1 |
| Dnajb2 | Bcl2l11 | Erp44 | Bbc3 | Erlin2 | Xbp1 | Jkamp | Rnf5 |
| Rhbdd1 | Ddrgk1 | AI314180 | Psmc4 | Ppp2cb | Erlec1 | Sel1l | Atf6b |
| Bcl2 | Tmx4 | Jun | Bax | Ubxn8 | Stc2 | Psmc1 | Bag6 |
| Ubxn4 | Trib3 | Casp9 | Ppp1r15a | Casp3 | Trp53 | Atxn3 | Flot1 |
| Yod1 | H13 | Fbxo6 | Vimp | Pik3r2 | Alox15 | Derl1 | Eif2ak2 |
| Ppp1rl5b | Edem2 | Fbxo2 | Rnf121 | Amfr | Derl2 | Rnf139 | Pmaip1 |
| Fam129a | Cebpb | Ube4b | Anks4b | Herpud1 | Trim25 | Foxred2 | Tmx3 |
| Edem3 | Ptpn1 | Ube2j2 | Ern2 | Aars | Cdk5rap3 | Pla2g6 | Syvn1 |
| Atf6 | Vapb | Psmc2 | Atp2a1 | Selk | Ccdc47 | Atf4 | Erlin1 |
| Ufc1 | Srpx | Tmub1 | Brsk2 | Ero1l | Psmc5 | Ep300 | |
| Atf3 | Aifm1 | Tmem129 | Ins2 | Psmc6 | Ern1 | Tmbim6 | |
| Man1b1 | Ubqln2 | Wfs1 | Ccnd1 | Trim13 | Nploc4 | Txndc11 | |
| Tor1a | Mbtps2 | Ube2k | Map3k5 | Dnajc3 | P4hb | Sdf2l1 | |
| Hspa5 | Usp13 | Tbl2 | Nrbf2 | Casp4 | Txndc5 | Ufd1l | |
| Dab2ip | Ufm1 | Get4 | Derl3 | Casp12 | Faf2 | Eif2b5 | |
| Nfe2l2 | Serp1 | Bhlha15 | Ube2g2 | Scamp5 | Ubqln1 | Nrros | |
| Dnajc10 | Creb3l4 | Creb3l2 | Tmem259 | Pml | Atg10 | Pdia5 | |
| Psmc3 | Tmem67 | Pdia4 | Creb3l3 | Parp16 | Thbs4 | Gsk3b | |
| Creb3l1 | Ufl1 | Eif2ak3 | Hsp90b1 | Nck1 | Col4a3bp | Park2 | |
| Thbs1 | Ube2j1 | Rnf103 | Apaf1 | Uba5 | Pik3r1 | Stub1 | |
| Eif2ak4 | Vcp | Aup1 | Ifng | Usp19 | Pdia6 | Pdia2 | |

Epithelial Identity

| | | | | | | | |
|---|---|---|---|---|---|---|---|
| Cdh1 | Cldn3 | Cldn7 | Ocln | Crb3 | Krt19 | Dsp | Pkp1 |
| Tgm1 | Cldn4 | Cldn11 | Epcam | Krt8 | Pkp3 | | |

ECM Rearrangement

| | | | | | | | |
|---|---|---|---|---|---|---|---|
| Sulf1 | Creb3l1 | B4galt1 | Mia | Atxn1l | Adamts2 | Tnfrsf11b | Cyp1b1 |
| Col19a1 | Hsd17b12 | Reck | Spint2 | Crispld2 | Wnt3a | Col14a1 | Fshr |
| Col3a1 | Wt1 | Tgfbr1 | Aplp1 | Foxf1 | Mfap4 | Has2 | Mkx |
| Col5a2 | Grem1 | Col27a1 | Hpn | Foxc2 | Serpinf2 | Ptk2 | Lox |
| Fn1 | Spint1 | P3h1 | Klk4 | Agt | Vtn | Scx | Hpse2 |
| Ihh | Cst3 | Hspg2 | Acan | Exoc8K | Nf1 | Fbln1 | Kazald1 |
| Col4a4 | Fkbp1a | Vwa1 | Serpinh1 | Ero1l | Col1a1 | Adamts20 | Nfkb2 |
| Col4a3 | Mmp9 | Dnajb6 | Apbb1 | Lgals3 | Ramp2 | Col2a1 | |
| Serpinb5 | Sulf2 | Emilin1 | Ilk | Ripk3 | Gfap | Myh11 | |
| Fmod | Atp7a | Mpv17 | Ric8 | Loxl2 | Sox9 | Ccdc80 | |
| Elf3 | Nox1 | Apbb2 | Muc5ac | Lcp1 | Ero1lb | Abi3bp | |
| Lamc1 | Col4a6 | Pdgfra | Ctgf | Mmp13 | Nid1 | App | |
| Tnr | Prdx4 | Ambn | Nr2e1 | Mmp20 | Foxf2 | Serac1 | |
| Dpt | Gpm6b | Dmp1 | Nepn | Col5a3 | Foxc1 | Plg | |
| Ddr2 | Egfl6 | Ibsp | P4ha1 | Smarca4 | Ripk1 | Smoc2 | |
| Olfml2b | Postn | Tfipl1 | Spock2 | Aplp2 | Tfap2a | Has1 | |
| Tgfb2 | Rxfp1 | Eln | Adamts14 | Mpzl3 | Ecm2 | Noxo1 | |
| Itga8 | Sfrp2 | Plod3 | Mmp11 | Thsd4 | B4galt7 | Col11a2 | |
| Adamtsl2 | Hapln2 | Col1a2 | Col18a1 | Anxa2 | Tgfbi | Tnxb | |
| Col5a1 | Ctss | Ndnf | Myf5 | Myo1e | Pxdn | Tnf | |
| Pomtl | Adamtsl4 | Vhl | Col4a1 | Nphp3 | Smoc1 | 2300002M2Rik | |
| Eng | St7l | Mfap5 | Csgalnact1 | Dag1 | Ltbp2 | Flot1 | |
| Lmx1b | Col11a1 | Ercc2 | Comp | Lamb2 | Flrt2 | Hsp90ab1 | |
| Gsn | Npnt | Bcl3 | Gfod2 | Kif9 | Fbln5 | Wash1 | |
| Olfml2a | Cyr61 | Tgfb1 | Has3 | Sh3pxd2b | Egflam | Vit | |

Apoptosis

| | | | | | | | |
|---|---|---|---|---|---|---|---|
| Ercc5 | Procr | Slc35d1 | Ldhb | Zfp365 | Zbtb16 | Sphk1 | Abcc5 |
| Serpinb5 | Blcap | Plk3 | Lrmp | Prmt2 | Rps27l | Rhbdf2 | Trp63 |
| Inhbb | Ada | Rnf19b | Tm7sf3 | Mknk2 | Mapkapk3 | Baiap2 | Fam162a |
| Steap3 | Fgf13 | Sfn | Tgfb1 | Dram1 | Ip6k2 | Dcxr | App |
| Btg2 | Irak1 | Fuca1 | Sertad3 | Apaf1 | Tcn2 | Hist1h1c | Rab40c |
| Phlda3 | Tspyl2 | Epha2 | Cebpa | Btg1 | Lif | Ninj1 | Bak1 |
| Tnni1 | Sat1 | Wrap73 | Klk8 | Mdm2 | Upp1 | Nol8 | Def6 |
| Rgs16 | Zmat3 | Mxd4 | Bax | Ddit3 | Ccng1 | F2r | Cdkn1a |
| Ier5 | Hspa4l | Rchy1 | Ppp1r15a | Gls2 | Cyfip2 | Ankra2 | Tap1 |
| Slc19a2 | Slc7a11 | Iscu | Rpl18 | Dgka | Gnb2l1 | Plk2 | Ier3 |
| Adck3 | Tm4sf1 | Triap1 | Aen | Cdkn2aip | Hint1 | Sdc1 | Polh |
| Ephx1 | Rap2b | Prkab1 | Rrp8 | Hmox1 | Gm2a | Gpx2 | Ccnd3 |
| Ptpn14 | Fbxw7 | Trafd1 | Ccp110 | Rrad | Hist3h2a | Zfp36l1 | Hbegf |
| Atf3 | S100a4 | Pom121 | Nupr1 | Cdh13 | Alox8 | Fos | Hdac3 |
| Notch1 | S100a10 | Pdgfa | Ptpre | Osgin1 | Trp53 | Ccnk | Rad9a |
| Rxra | Txnip | Gadd45a | Hras | Cgrrf1 | Tax1bp3 | Jag2 | Ctsf |
| Ralgds | Nhlh2 | Vamp8 | Eps8l2 | Abhd4 | Traf4 | Ndrg1 | Slc3a2 |
| Ak1 | Dnttip2 | Retsat | Ctsd | Kif13b | Cdk5rl | Pmm1 | Fas |
| Stom | Clca2 | Tprkb | Cd81 | Rb1 | Ppm1d | Plxnb2 | |
| Ddb2 | Wwp1 | Tgfa | Perp | Nudt15 | Rad51c | Vdr | |
| Cd82 | Klf4 | Mxd1 | Rps12 | Tsc22d1 | Tob1 | Csrnp2 | |
| Il1a | Ikbkap | Sec61a1 | Tpd52l1 | Casp1 | Krt17 | Acvr1b | |
| Pcna | Cdkn2a | Xpc | Sesn1 | St14 | Hexim1 | Sp1 | |
| Bmp2 | Cdkn2b | Ccnd2 | Foxo3 | Ei24 | Fdxr | Abat | |
| Trib3 | Jun | H2afj | Ddit4 | Vwa5a | Itgb4 | Socs1 | |

SASP

| | | | | | | | |
|---|---|---|---|---|---|---|---|
| Il6 | Cxcl2 | Csf2 | Fgf7 | Igfbp4 | Mmp14 | Icam3 | Egfr |
| Il7 | Cxcl3 | Mif | Vegfa | Igfbp6 | Timp2 | Tnfrsf11b | Fn1 |
| Il1a | Ccl8 | Areg | Ang | Igfbp7 | Serpine1 | Tnfrsf1a | |
| Il1b | Ccl13 | Ereg | Kitl | Mmp1 | Serpinb2 | Tnfrsf1b | |
| Il13 | Ccl3 | Nrg1 | Cxcl12 | Mmp3 | Plat | Tnfrsf10b | |
| Il15 | Ccl20 | Egf | Pigf | Mmp10 | Plau | Fas | |
| Cxcl15 | Ccl16 | Fgf2 | Igfbp2 | Mmp12 | Ctsb | Plaur | |
| Cxcl1 | Ccl26 | Hgf | Igfbp3 | Mmp13 | Icam1 | Il6st | |

Neural Identity

| | | | | | | | |
|---|---|---|---|---|---|---|---|
| Vtn | Zeb2 | Sox1 | Pax6 | Sox2 | Msx1 | Atoh1 | Tubb3 |
| Ednrb | Hes5 | Neurod1 | Cdh2 | Id2 | Msi1 | Rbfox3 | |
| Sox21 | Fabp7 | Pax3 | Sox9 | Hoxb1 | Msi2 | Map2 | |

Placental Identity

| | | | | | | | |
|---|---|---|---|---|---|---|---|
| 4933433p14rik | Dusp9 | Pkp2 | Tnfrsf23 | Serpinb9d | Krt18 | 1600014k23rik | Hapln3 |
| Esx1 | H19 | 9630050e16rik | Sos1 | Plekhh1 | Nrn1l | Tbrg1 | Fam176a |
| Afap1 | Tmem37 | Pvrl2 | Dlx3 | 2210011c24rik | Sfi1 | Slit1 | Pdlim1 |
| Zfyve21 | Mmp15 | Zfp568 | Ippk | Cd320 | Tlr5 | A730090h04rik | Ube2q2 |
| Erv3 | Fam101b | Vtcn1 | Htr2b | Ccnjl | Rhou | 4931406p16rik | Au018091 |
| Atg12 | Phf16 | Il6ra | Dusp16 | Entpd2 | Arhgef6 | Opn3 | Bdkrb2 |
| Las1l | 4930422n03rik | Foxo4 | Cdc73 | Il1r2 | Tmem185b | Pdia4 | E130203b14rik |
| Rbp1 | Ada | Hsp90b1 | 1700025g04rik | Sfmbt2 | Tram2 | B930054o08 | S100g |
| Prl2b1 | Mmp1a | Prl7c1 | Prl4a1 | 1700011m02rik | Cited1 | 170031f05rik | 4933402e13rik |
| Prl3d1 | Gpr126 | Prl6a1 | Zfp655 | Plekha7 | Cited2 | Inhba | Dapk2 |
| Rnf2 | Arf2 | Cdh5 | Slc13a4 | Sfrp5 | Zfand2a | Inhbb | Gm11985 |
| Sct | Tinagl1 | Fgd6 | Ceacam14 | Ppp1r3f | Krt25 | Helz | Fndc3b |
| Mrgprg | Mfi2 | Cysltr2 | Ceacam15 | Obsl1 | Klk4 | Sele | Twsg1 |
| Aa763515 | Rpn2 | Rhox6 | Trap1a | Slc23a3 | Tnfrsfl1b | Pdia6 | Aldh1a3 |
| Tfpi | Abhd2 | Cdh3 | Ceacam12 | Tmem87b | 2010204k13rik | Pdia5 | Lnx2 |
| Etos1 | Hrct1 | Spp2 | Gm16515 | Epas1 | Tor1aip2 | Creb3 | Taf7 |
| Slc5a6 | Adm | Zim1 | Ceacam13 | Ccdc68 | Fmr1nb | Efna1 | Ai844869 |
| 1600025m17rik | Abhd6 | Flnb | 4930447f24rik | Kdelr2 | Ctsr | Dlg5 | Clec12b |
| Gm9 | Slc7a1 | Rbbp7 | Gzmd | Pramef12 | Ctsq | Procr | Prkcsh |
| Creb3l2 | Tead4 | Map3k7 | Foxj2 | Lrp8 | Prl8a2 | Fgfr1 | Lama5 |
| Bbx | Mbnl3 | Rhox9 | Fbxl19 | Pard6b | Ctsm | Gnb4 | Tchh |
| Prl3c1 | Gpr1 | Whsc1l1 | Gzmc | Peg10 | Prl8a1 | 2310030g06rik | Lama1 |
| Mta3 | 2900057e15rik | Slc38a1 | Gzmf | N4bp2 | Ctsj | Gcm1 | Rps6ka6 |
| Prl2a1 | Ldoc1 | 1600012p17rik | Gzme | Pla2g4e | Mpzl1 | Psg18 | Vhl |
| Gm9112 | Adam19 | Adra2b | Gzmg | Fam78b | Stra6 | Golt1b | Eps8l2 |
| Afap1l2 | Rybp | Pgf | Patl2 | Arrdc3 | Bcap31 | Psg19 | Polg |
| Erlin2 | Col4a1 | 1200009i06rik | 3830417a13rik | Pla2g4d | Creg1 | Psg16 | |
| Pard3 | Fndc3c1 | Mfsd7c | Tspan14 | Rassf8 | Tcfap2c | Slc2a1 | |
| Aif1l | Col4a2 | Esam | Hand1 | Au015836 | Prl7b1 | Psg17 | |
| Dmrtc1a | 4930502e18rik | Gpr107 | Atxn10 | Csnk1e | Ghrh | Htra3 | |
| 4932442l08rik | Pkn2 | Au015791 | Mgat4a | Stag1 | 4930486l24rik | Klhl13 | |
| Gjb2 | Rlim | Arhgap8 | Unc50 | Vnn1 | Neurog2 | Ets2 | |
| Gjb5 | 160001l5i10rik | Ankrd17 | Il2rb | Tchhl1 | 5430425j12rik | Nppc | |
| Slco5a1 | Afp | Cul7 | Ceacam11 | Pla1a | Prl7a1 | Tgm1 | |
| Wdr61 | Tmem140 | 2310067p03rik | Plekhg1 | Slc45a4 | Prl7a2 | Tmem108 | |
| Kitl | Fstl3 | Irs3 | Prl3b1 | Tex264 | Mir1199 | Usp53 | |
| 9430027b09rik | Ing4 | Prl5a1 | Folr1 | Pcdh12 | Tbc1d10a | Mark3 | |
| Tfrc | Taf7l | Fntb | A830080d01rik | Ctr9 | Ralbp1 | Cbx8 | |
| Slc6a2 | Sult1e1 | Tceanc | Blzf1 | Ccr1l1 | Pdgfra | Hspa5 | |
| Wdr45 | Olr1 | Lepr | Zfp667 | Htatsf1 | Morc4 | Spats2 | |
| Zxda | 2610019f03rik | Tnfrsf9 | Flt1 | 9030409g11rik | Rarres2 | Limk2 | |
| Prdx4 | F11 | Papola | Usp27x | Tspan9 | Arid3a | Mkl2 | |
| Fam122b | Fbxw8 | Srd5a1 | Hdac4 | Rassf6 | Lifr | Shroom4 | |
| Zxdb | Sema4c | C1qtnf1 | Itgb3 | 4631402f24rik | Shisa3 | Shroom1 | |
| Zxdc | Ctnnbip1 | Slc38a4 | Sri | A2m | Uevld | Pou2f3 | |
| Pip5k1a | Tfpi2 | Angpt4 | Sema3f | Rimklb | Scnn1b | Acvr2b | |
| Plac1 | Zbtb10 | Ctla2a | Prl3a1 | Loc100504569 | Dnajb12 | Rbms2 | |
| Igf2as | Mitf | 9930012k11rik | Bahd1 | Apob | Brwd3 | Atg4b | |
| Usp9x | Gpr50 | Mical3 | Sin3b | Tmem150a | Hhipl1 | Pappa2 | |
| Psg28 | Hic2 | Apoa4 | Gm2a | 9130404d08rik | Fbln7 | Rbm25 | |
| Bmp8b | Tpbpb | Cul4b | Serpinb9g | Prl8a6 | Masp1 | Gm4793 | |
| Fn1 | Slc9a6 | 3632454l22rik | Bend4 | Cts6 | Nrk | Nid1 | |
| Psg23 | Prl7d1 | Psg-ps1 | Bend5 | Prl8a8 | Pvr | Uba6 | |
| Bmp8a | Tpbpa | Lcor | Serpinb9b | Prl8a9 | Atp2c1 | Lamc1 | |
| Psg21 | Slco2a1 | Tnfrsf22 | Serpinb9c | Cts3 | Amot | Slc40a1 | |

X reactivation

| | | | | | | | |
|---|---|---|---|---|---|---|---|
| Gm21950 | Slc9a7 | Rhox3h | Slitrk4 | Fam47c | Zdhhc15 | Bhlhb9 | Samt1 |
| Gm21364 | Rp2 | Rhox2h | Ctag2 | Gm7173 | 1700121L16Rik | Gprasp2 | 4921511M17Rik |
| Gm14346 | Jade3 | Rhox5 | 4930447F04Rik | Mageb16 | Magee2 | Arxes2 | Gm10057 |
| Gm14345 | Rgn | Rhox6 | Slitrk2 | Gm26775 | Pbdc1 | Arxes1 | Gm15140 |
| Gm14351 | Ndufb11 | Rhox7a | 1700036O09Rik | Tmem47 | Magee1 | Bex2 | 4930524N10Rik |
| Gm3701 | Rbm10 | Rhox8 | Gm1140 | 4930595M18Rik | 5330434G04Rik | Nxf3 | Samt4 |
| Gm3706 | Uba1 | Rhox7b | Gm14692 | Dmd | Cypt2 | Bex4 | Samt2 |
| Gm14347 | Cdk16 | Rhox9 | 4933436I01Rik | Tsga8 | Fgf16 | Tceal8 | Cldn34b1 |
| Gm10921 | Usp11 | Btg1-ps1 | Fmr1os | Fthl17a | Atrx | Tceal5 | Magea6 |
| Gm10922 | Araf | Btg1-ps2 | Fmr1 | Tab3 | Magt1 | Bex1 | Magea3 |
| Gm3750 | Syn1 | Rhox10 | Fmr1nb | Gk | Cox7b | Tceal7 | Magea8 |
| Gm3763 | Timp1 | Rhox11 | Gm14698 | Gm14764 | Atp7a | Wbp5 | Magea2 |
| Mycs | Cfp | Rhox12 | Gm6812 | Gm14762 | Tlr13 | Ngfrap1 | Magea5 |
| Gm14374 | Elk1 | Rhox13 | Gm14705 | 5430427O19Rik | Pgk1 | Kir3dl2 | Magea1 |
| Nudt11 | Uxt | Zbtb33 | Aff2 | Samt3 | Taf9b | Kir3dl1 | Cldn34b2 |
| AU022751 | Zfp182 | Tmcm255a | 1700111N16Rik | Nr0b1 | Fnd3c2 | Tceal3 | Sat1 |
| Nudt10 | Spaca5 | Atp1b4 | 1700020N15Rik | Mageb4 | Fndc3c1 | Tceal1 | Acot9 |
| Bmp15 | Zfp300 | Lamp2 | Ids | Il1rapl1 | Cysltr1 | Morf4l2 | Prdx4 |
| Shroom4 | Ssxa1 | Gm7598 | 1110012L19Rik | Gm27000 | Gm5127 | Glra4 | Ptchd1 |
| Dgkk | Gm21876 | Cul4b | 4930567H17Rik | Pet2 | Zcchc5 | Plp1 | Gm15156 |
| Ccnb3 | 4930453H23Rik | Mcts1 | BC023829 | 4932429P05Rik | Lpar4 | Rab9b | Gm15155 |
| Akap4 | Gm6938 | C1galt1c1 | Mamld1 | 4930415L06Rik | P2ry10 | H2bfm | Phex |
| Clcn5 | Gm26593 | Gm14565 | Mtm1 | Gm44 | A630033H20Rik | Tmsb15l | Sms |
| Usp27x | Agtr2 | 6030498E09Rik | Mtmr1 | Gm14773 | Gpr174 | Tmsb15b2 | Mbtps |
| Ppp1r3f | Slc6a14 | Cypt15 | Cd99l2 | Mageb2 | Itm2a | Tmsb15b1 | Yy2 |
| Ppp1r3fos | Gm28269 | Cypt14 | Gm16189 | Gm5072 | Tbx22 | Slc25a53 | Smpx |
| Foxp3 | Gm28268 | Gria3 | Hmgb3 | Gm8914 | 2610002M06Rik | Zcchc18 | Gm15169 |
| Ccdc22 | Klhl13 | Thoc2 | Gpr50 | 1700084M14Rik | Fam46d | Fam199x | Klhl34 |
| Cacna1f | Wdr44 | Xiap | Vma21 | Gm14781 | Gm732 | Esx1 | Cnksr2 |
| Syp | Gm4907 | Stag2 | Gm1141 | Mageb5 | Gm379 | Il1rapl2 | Rps6ka |
| Gm14703 | Gm4985 | Gm43337 | Prrg3 | Mageb1 | Brwd3 | Tex13a | Eif1ax |
| Prickle3 | Gm27192 | Sh2d1a | Fate1 | Mageb18 | Hmgn5 | Nrk | Map7d2 |
| Plp2 | Gm5934 | Tenm1 | Cnga2 | Gm5941 | Sh3bgrl | Serpina7 | A830080D01Rik |
| Magix | Gm4297 | Gm362 | Magea4 | 1700003E24Rik | Gm6377 | 4930513O06Rik | Sh3kbpl |
| Gpkow | Gm5935 | Dcaf12l2 | Gabre | BC061195 | RP23-240M8.2 | 4933428M09Rik | Map3k15 |
| Wdr45 | Gm5169 | Dcaf12l1 | Magea10 | Arx | Pou3f4 | Mum1l1 | Pdha1 |
| RP23-109E24.10 | Grn1993 | Prr32 | Gabra3 | Pola1 | Cylc1 | Trap1a | Adgrg2 |
| Praf2 | E330010L02Rik | 4930515L19Rik | Gabrq | Pcyt1b | Gm10112 | D330045A20Rik | Gm15241 |
| Ccdc120 | Gm5168 | Actrt1 | Cetn2 | Pdk3 | Rps6ka6 | Rnf128 | Phka2 |
| Tfe3 | Gm2012 | Gm129242 | Nsdhl | AU015836 | Hdx | TbCld8b | Gm15243 |
| Gripap1 | Gm2030 | Smarca1 | Gm14684 | Gm14798 | RP23-466J17.3 | Gm15013 | Ppef1 |
| Kcnd1 | Slx | Ocr1 | Zfp185 | Zfx | Tex16 | Ripply1 | Rs1 |
| Otud5 | Gm14525 | Apln | Pnma5 | Eif2s3x | 4933403O08Rik | Cldn2 | Cdkl5 |
| Pim2 | Gm6121 | Xpnpep2 | Pnma3 | Klhl15 | Apool | Morc4 | Gja6 |
| Slc35a2 | Gm10230 | Sash3 | Xlr4a | Fam90a1b | Satl1 | Rbm41 | Scml2 |
| Pqbp1 | Gm2101 | Zdhhc9 | Xlr3a | Apoo | 2010106E10Rik | Nup62cl | Gm15262 |
| Timm17b | Gm10058 | Utp14a | Xlr5a | Gm14827 | Zfp711 | Pih1h3b | Rai2 |
| Gm10491 | Gm2117 | 9530027J09Rik | Gm14685 | Maged1 | Pof1b | Gm15046 | Scml1 |
| Gm10490 | Gm4836 | Bcorl1 | DXBay18 | Gspt2 | Gm14936 | Frmpd3 | Gm15205 |
| Pcsk1n | Gm10147 | Elf4 | Xlr5b | Zxdb | Chm | Prps1 | Nhs |
| Eras | Gm2165 | Aifm1 | Spin2d | RP23-9K14.6 | Dach2 | Tsc22d3 | Gm15202 |
| Hdac6 | Gm10096 | Rab33a | X1r3b | Gm26617 | K1h14 | Mid2 | Reps2 |
| Gata1 | Gm2200 | Zfp280c | X1r4b | Spin4 | Ube2dnl1 | Eif2c5 | Pbbp7 |
| Glod5 | Gm26818 | Slc25a14 | F8a | Arhgef9 | Ube2dnl2 | Tex13 | Txlng |
| Gm14820 | Gm3669 | Gpr119 | X1r4c | Amer1 | 4930555B12Rik | Vsig1 | Syap1 |
| Suv39h1 | Gm10488 | Rbmx2 | X1r3c | Asb12 | Cpxcr1 | Psmd10 | Ctps2 |
| Was | E330016L19Rik | Gm595 | X1rSc | Zc4h2 | H2afb2 | Atg4a | S100g |
| Wdr13 | Gm14632 | Enox2 | RP23-95K12.13 | Zc3h12b | Gm14920 | Col4a6 | Grpr |
| Rbm3 | Gm7437 | Gm14696 | Zfp275 | 1700010D01Rik | Gm28579 | Col4a5 | Rnf138rt1 |
| Rbm3os | Gm14974 | Gm14697 | Gm18336 | Las1l | Tgif2lx2 | Irs4 | Ap1s2 |
| Tbc1d25 | Gm10487 | Arhgap36 | Gm26726 | Msn | Tgif2lx1 | Gm15295 | Zrsr2 |
| Ebp | Gm21447 | Olfr1320 | Zfp92 | F630028O10Rik | Gm14929 | Gm15294 | Car5b |
| Porcn | Spin2f | Olfr1321 | Trex2 | Vsig4 | Pabpc5 | Gm15298 | Siah1b |
| Ftsj1 | Gm2784 | Igsf1 | Haus7 | Hsf3 | Pcdh11x | Gucy2f | Tmem27 |
| Slc38a5 | Gm2777 | Olfr1322 | Bgn | Heph | H2afb3 | Nxt2 | Ace2 |
| Ssxb10 | Gm21883 | Olfr1323 | Atp2b3 | Gpr165 | Nap1l3 | Kcne1l1 | Bmx |
| Ssxb9 | Spin2e | Olfr1324 | Dusp9 | Pgr15l | Gm17521 | Acsl4 | Pir |
| Ssxb1 | Gm21608 | Stk26 | Pnck | Eda2r | Cldn34c1 | Tmem164 | Figf |
| Ssxb2 | Gm21637 | Frmd7 | Slc6a8 | Ar | Astx6 | Ammecr1 | Piga |
| Gm14459 | Gm21645 | Rap2c | Bcap31 | Ophn1 | Srsx | Rgag1 | Asb11 |
| Ssxb6 | Gm2799 | Mbnl3 | Abcd1 | Yipf6 | Gm17577 | Chrdl1 | Asb9 |
| Ssxb3 | GmcI1l | Hs6st2 | Plxnb3 | Stard8 | Gm14951 | Pak3 | Mospd2 |
| Ssxb8 | Gm5926 | Usp26 | Srpk3 | Efnb1 | Astx2 | Capn6 | Fancb |
| Ssx9 | Gm21951 | 1700080O16Rik | Idh3g | GM14812 | Gm17412 | Dcx | Gm17604 |
| Ssxb5 | Gm21657 | Gpc4 | Ssr4 | Gm14809 | Cldn34c2 | A730046J19Rik | Glra2 |
| Gm6592 | Gm21789 | Gpc3 | Pdzd4 | Gm14808 | Gm14950 | Alg13 | Gemin8 |
| Gm5751 | Gm2825 | Gm14582 | L1cam | Pja1 | Gm17467 | Trpc5 | Gpm6b |
| B630019K06Rik | Spin2-ps6 | A630012P03Rik | Arhgap4 | Tmem28 | Cldn34c3 | Trpe5os | Ofd1 |
| Fthl17b | Gm2863 | Ccdc160 | Avpr2 | Eda | Astx5 | Zcchc16 | Trappc2 |
| Fthl17c | Gm2854 | Phf6 | Naa10 | Awat2 | Vmn2r121 | Lhfpl1 | Rab9 |
| Fthl17d | Gm2913 | Hprt | Renbp | Otud6a | Astx1a | Amot | Tceanc |
| Fthl17e | Gm2927 | Gm28730 | Hefc1 | Igbp1 | Gm17584 | Htr2c | Egfl6 |
| Fthl17f | Gm2933 | Plac1 | Irak1 | Dgat2l6 | Astx4a | Il13ra2 | Gm15226 |
| 4930402K13Rik | Gm2964 | Fam122b | Mecp2 | Awat1 | Gm17469 | Lrch2 | Gm1720 |
| Lancl3 | Gm21870 | Fam122c | Opn1mw | P2ry4 | Astx4b | Gm15128 | Gm15230 |
| Gm14862 | Gm21681 | Mospd1 | Tex28 | Arr3 | Astx1b | Gm15080 | Gm8817 |
| Xk | Spin2g | Etd | Tktl1 | Pdzd11 | Gm17361 | Gm15107 | Gm15232 |
| 1700012L04Rik | Gm21699 | Gm14597 | Flna | Kif4 | Gm21616 | Gm15114 | Gm15228 |
| Gm14501 | Gm14552 | Cxx1c | Emd | Gdpd2 | Astx4c | Gm8334 | Tmsb4x |
| Cybb | Gm10486 | Cxx1a | Rpl10 | Gm14902 | Gm17693 | Gm15127 | Tlr8 |
| Gm5132 | Gm2309 | Cxx1b | Dnase1l1 | Dlg3 | Astx1c | Luzp4 | Tlr7 |
| Dynlt3 | Gm14553 | 4930502E18Rik | Taz | Texl1 | Gm17522 | Gm15099 | Prps2 |
| Hypm | Gm14819 | 1700013H16Rik | Atp6ap1 | Slc7a3 | Astx4d | Ott | Gm15239 |
| 4930557A04Rik | Dock11 | Zfp36l3 | Gdi1 | Snx12 | Gm17267 | Gm15092 | Frmpd4 |
| Sytl5 | Il13ra1 | Xlr | Fam50a | Foxo4 | Astx3 | Gm15093 | Msl3 |
| Srpx | Zcchc12 | Gm16405 | Plxna3 | Gm614 | 4932411N23Rik | Gm15100 | Arhgap6 |
| Rpgr | Lonrf3 | Gm16430 | Lage3 | Gm20489 | Gm382 | Gm15085 | Gm15261 |
| Otc | Gm6268 | Slxl1 | Ubl4a | Il2rg | 4921511C20Rik | Gm15086 | Amelx |
| Tspan7 | Gm14569 | 3830403N18Rik | Slc10a3 | Med12 | Cldn34c4 | Gm10439 | Hccs |
| Gm10489 | Pgrmc1 | Gm773 | Fam3a | Nlgn3 | 4930558G05Rik | Gm15097 | Gm15245 |
| Mid1ip1 | Akap17b | 1600025M17Rik | Ikbkg | Gjb1 | Diaph2 | Gm15091 | Mid1 |
| Gm14493 | Slc25a43 | Zfp449 | G6pdx | Zmym3 | Pcdh19 | Gm15104 | 4933400A11Rik |
| Gm14483 | Slc25a5 | Gm2155 | Gm6880 | Nono | Gm26851 | Tmem29 | Gm15726 |
| Gm14474 | Gm14549 | Smim1ol2a | Olfr1326-ps1 | Itgb1bp2 | Tnmd | Apex2 | Gm15247 |
| Gm14477 | 2310010G23Rik | Gm2174 | Olfr1325 | Taf1 | Tspan6 | Alas2 | Gm21887 |
| Gm14476 | C330007P06Rik | Ddx26b | Gm5640 | Ogt | Srpx2 | Pfkfb1 | Asmt |
| Gm14484 | Ube2a | Gm10477 | Gm6890 | Cxcr3 | Sytl4 | Tro | |
| Gm14479 | Nkrf | Gm648 | Gm5936 | Gm4779 | Cstf2 | Maged2 | |
| Gm14482 | Gm15008 | Mmgt1 | Gab3 | 8030474K03Rik | Nox1 | GM27191 | |
| Gm14478 | 43349 | Slc9a6 | Dkc1 | Nhsl2 | Xkrx | Gnl31 | |
| Gm14475 | Sowahd | Fhl1 | Mpp1 | Rgag4 | Arl3a | Fgd1 | |
| Gm4906 | Rpl39 | Mtap7d3 | Smim9 | Pin4 | Trmt2b | Tsr2 | |
| Bcor | Upf3b | Adgrg4 | F8 | Ercc6l | Tmem35 | Gm15138 | |
| Gm14635 | Nkap | Brs3 | Fundc2 | Rps4x | Cenpi | Wnk3 | |
| Atp6ap2 | Akap14 | Htatsf1 | Cmc4 | Cited1 | Drp2 | A230072E10Rik | |
| 1810030O07Rik | Ndufa1 | Vgl11 | Mtcp1 | Hdac8 | Taf7l | Fam120c | |
| Med14 | Rnf113a1 | Gm14718 | Brcc3 | Phka1 | Timm8a1 | Phf8 | |
| Usp9x | Gm9 | Cd4olg | Vbp1 | Gm9112 | Btk | Huwe1 | |
| 2010308F09Rik | Rhox1 | Arhgef6 | Gm15384 | Dmrtc1b | Rpl36a | Hsd17b10 | |
| Ddx3x | Rhox2a | Rbmx | Rab39b | Dmrtc1c1 | Gla | Ribc1 | |
| Nyx | Rhox3a | Gm364 | Gm15063 | Dmrtc1c2 | Hnrnph2 | Smc1a | |
| Cask | Rhox4a | Gpr101 | Pls3 | 1700031F05Rik | Armcx4 | Iqsec2 | |
| Gpr34 | Rhox3a2 | Zic3 | Gm14715 | Dmrtc1a | Anmcx1 | Kdm5c | |
| Gpr82 | Rhox4a2 | 4930550L24Rik | Gm14707 | 1700011M02Rik | Armcx6 | Kantr | |
| Gm5382 | Rhox2b | Fgf13 | Gm14717 | Nap1l2 | Armcx3 | Tspyl2 | |
| Gm14505 | Rhox4b | F9 | Cldn34b3 | Cdx4 | Armcx2 | Gpr173 | |
| Drr1 | Rhox2c | Mcf2 | Cldn34b4 | Chic1 | Nxf2 | Cldn34a | |
| Cypt1 | Rhox3c | Atp11c | Cldn34d | Gm26952 | Zmat1 | Shroom2 | |
| Maoa | Rhox4c | Gm7073 | Tbl1x | Tsx | Gm15023 | Gpr143 | |
| Maob | Rhox2d | Gm14661 | Prkx | Gm26992 | Tceal6 | Usp51 | |
| Ndp | Rhox4d | Sox3 | Gm14742 | Tsix | Pramel3 | Mageh1 | |
| Efhc2 | Rhox2e | Gm14662 | Pbsn | Xist | Gm5128 | Foxr2 | |
| Fundc1 | Rhox3c | Gm14664 | Gm14744 | Jpx | Gm7903 | Rragb | |
| Dusp21 | Rhox4e | Cdr1 | 5430402E10Rik | Ftx | AV320801 | Klf8 | |
| Kdm6a | Rhox2f | Ldoc1 | Obp1a | Zcchc13 | Nxf7 | Ubqln2 | |
| 4930578C19Rik | Rhox3f | 4933402E13Rik | Gm5938 | Slc16a2 | Prame | Cypt3 | |
| Gm26652 | Rhox4f | 4931400O07Rik | Obp1b | Rlim | Tcp11x2 | Kctd12b | |
| BC049702 | Rhox3g | 1700019B21Rik | Gm14743 | C77370 | Tmsb15a | RP23-106P7.5 | |
| Chst7 | Rhox2g | Gm6760 | 4930480E11Rik | Abcb7 | Armcx5 | 2210013O21Rik | |
| | Rhox4g | 3830417A13Rik | Prrg1 | Uprt | Gprasp1 | Spin2c | |

XEN

| | | | | | | | |
|---|---|---|---|---|---|---|---|
| Dab2 | Pdgfra | Gata6 | Fxyd3 | Sox17 | Lama1 | Gata4 | Krt8 |
| Fst | Pth1r | Foxq1 | Tet3 | Foxa2 | Lamb1 | | |

Trophoblast

| | | | | | | | |
|---|---|---|---|---|---|---|---|
| Ascl2 | Cdx2 | Esrrb | Grn | Lipg | Smad3 | Tfap2c | Gata3 |
| Bmp4 | Elf5 | Ets2 | Igf2 | Pcsk6 | Snai1 | Vav1 | Krt7 |
| Bmp8b | Eomes | Fgfr2 | Jade1 | Ptpra | Tead4 | Yap1 | Krt18 |

Trophoblast progenitors

| | | | | | | | |
|---|---|---|---|---|---|---|---|
| Rhox6 | Hmgn2 | Tuba1b | Immt | Rps21 | Ccnd3 | Mrpl54 | Ruvbl2 |
| Rhox9 | Odel | Cenpw | Smagp | Pdlim2 | Rpl5 | Rps26 | Ndufv1 |
| 3830417A13Rik | Klhl13 | Cct7 | Hnrnpa2b1 | Rpl24 | Nip7 | Ndufb9 | Polr2l |
| Gjb3 | Ncl | Sfn | Cox7b | Asf1a | Psma5 | Arpc1a | Asns |
| Gm9112 | Tyms | Fkbp4 | Snx10 | Eif4a3 | Spc24 | Rps28 | Prkrip1 |
| Hspb1 | Prss8 | Ndufbb | Stip1 | Ssb | Mdh2 | Prpg31 | 1700021F05Rik |
| Nup62cl | Atp5g3 | Snrpe | Rnf4 | Timm17a | Cep164 | Mrpl12 | Aimp1 |
| Ldoc1 | Dusp9 | Cenph | Gm648 | Mrpl18 | Cs | Epop | Rps7 |
| Hspe1 | Gmnn | Rad51 | Cct6a | Cenpk | Zc3h15 | Cct5 | Tra2b |
| Rhox12 | Rrm2 | Set | Snrpd2 | Dcakd | Pea15a | Pdap1 | Cox17 |
| Tex19.1 | Tbrg1 | Cd164 | Psmg2 | Hikeshi | Tsen15 | Ezh2 | Mrpl19 |
| Gjb5 | Cct3 | Cox6b1 | Tk1 | U2af1 | Ippk | Gpbp1 | Chchd4 |
| Sin3b | Nhp2 | Hnrnpdl | Rps5 | Acp1 | Thoc3 | Psme3 | Polr1d |
| 1700086L19Rik | Ppid | Lsm2 | Mtx2 | Tipin | Pithd1 | Ube2c | Ubfd1 |
| Ldhb | Ccna2 | Exoc314 | Phb | Fkbp3 | Pak1ip1 | Cbx1 | 2410015M20Rik |
| Krt19 | Anp32b | Dut | Hspa8 | Cdca3 | 1110038B12Rik | Gata2 | Tbcb |
| Hmgn5 | Cacybp | Pramef12 | mt-Nd5 | Tubb4b | Wdr18 | Nxf7 | Chchd1 |
| Trap1a | Chchd2 | Cd320 | Orc6 | Mycbp | Nol7 | Smc4 | Serbp1 |
| Plac1 | Phb2 | Snrpd3 | Dctpp1 | Apip | Tomm70a | Tfap2c | Hsph1 |
| Cdkn1c | Snrpf | Psmb7 | Sugt1 | Mdk | Snu13 | Creb3 | Xpo1 |
| Bex1 | Ran | Mcm7 | Wdr77 | Rpl14 | Psma2 | Clns1a | 2310033P09Rik |
| Fthl17a | Gale | Taf1d | Suclg1 | Cox7a2 | Eif2s2 | 1810022K09Rik | Prpf19 |
| Dbi | mt-Nd4 | H2afz | Ddx39 | Hnrnpc | Usmg5 | Eif2b1 | Apoo |
| Ube2a | Birc5 | Ndugfb2 | Polr2f | Sdr39u1 | Eif3e | Idh3a | Hagh |
| Dnaja1 | Tpm2 | Lyar | Rpl38 | Slc25a3 | Cops5 | Sae1 | Ndufa9 |
| Phactr1 | Hsd17b4 | Rbms2 | Rpa2 | Psma7 | Mrpl3 | Eif5a | Mrpl2 |
| Phlda2 | Rpl22l1 | Eif5b | Fmr1nb | Psmd12 | Mybbp1a | Fhl2 | Ndufb7 |
| Hand1 | Snrpd1 | Rbm8a | Gng12 | Cyc1 | Elp2 | Lap3 | Psmb1 |
| Selenoh | Hspa14 | Dynll1 | Tuba1c | Apex1 | 1110004F10Rik | Ncbp2 | Txndc9 |
| Rhox5 | Wfdc2 | Stmn1 | Aasdhppt | Rad23b | St13 | Eps8l2 | Hnrnpa1 |
| Atp5g1 | Rfc4 | Got2 | Pfdn6 | C1qbp | Tbca | Cdk4 | Ndufs7 |
| Hmgn1 | Rgcc | Cox7c | Hspa9 | Cox6c | Snrpa1 | Rfc3 | Farsb |
| Hat1 | Mfsd2a | Lsm6 | Eif1a | Txn1 | H2afv | Cdk1 | Cycs |
| Plet1 | Cct8 | Ccne2 | Pop5 | Med19 | Mcm7 | Mrps25 | Tmem11 |
| Gm9 | Ubxn1 | Sap18 | Nasp | Slirp | Tcp1 | Coq3 | Rps17 |
| Rbbp7 | Ddt | Liph | Xlr4b | G3bp1 | Atp1b1 | Med10 | Mrpl14 |
| Hspd1 | Dtymk | Pa2g4 | Snrpb2 | Ak2 | Aprt | Emd | Diablo |
| Mrfap1 | C430049B03Rik | Slc38a4 | Nop58 | Krt18 | Nup37 | Ptrh2 | Cox4i1 |
| Krt7 | Magoh | Irx3 | Uqcrc2 | Rsl1d1 | Hebp1 | Mrps18c | Pkp2 |
| Esam | Calm2 | Srsf3 | Cfdp1 | Csrp1 | Lsm8 | Med4 | Psmc2 |
| Krt8 | Mrps22 | Dpy30 | Hn1l | 1600025M17Rik | Mbd3 | Fam133b | Psmc1 |
| Fstl3 | Impdh2 | Hmgcl | Tsn | Rpp30 | Gtf3c6 | Crip2 | Slc25a4 |
| Ghrh | Brd3 | Cenpa | Psma6 | Mrpl38 | Rpa3 | Ndufa3 | Eloc |
| Ranbp1 | Fscn1 | Mgll | Ssrp1 | Emg1 | Cdc34 | Thap4 | Vma21 |
| Npm1 | 2610528J11Rik | Eef1g | Acaa1a | Cebpzos | Ndufb8 | Mrps16 | Mif |
| H19 | Zwint | Atp5cl | Rpf2 | Nsmce4a | Nap1l1 | Uchl3 | Timm13 |
| Sdc1 | Tmem37 | Imp4 | Lgals1 | Cct2 | Adgrf5 | Mea1 | |
| Rps4l | Ndufa5 | Cks2 | Psmd6 | Rps16-ps2 | Ptges3 | Psma3 | |
| mt-Nd1 | Eif2s1 | Rnd2 | Ap1m2 | Ruvbl1 | Polr2j | Timm10 | |
| Hsp90aa1 | Hsd17b2 | Knstm | Plpp1 | Arpp19 | Ndufa12 | Rrm1 | |
| Mbnl3 | Galk1 | Atp5fl | Ndufaf2 | Rpl27 | Cyb5b | Hnrnpd | |
| Htatsf1 | Cct4 | Skp1a | Cul1 | Dcun1d5 | Tmod3 | Tomm22 | |
| Hsp90ab1 | Cox5a | Igf2bp1 | Ndufal1 | Rpl18 | Ndufv2 | Ndufab1 | |
| Las1l | Dkkl1 | Mrpl21 | mt-Col | Mrpl15 | Ash2l | Aifm1 | |
| Ptma | Hmgb2 | Srsf7 | Tomm40 | Psma1 | Spc25 | Tfam | |
| mt-Cytb | Tubb5 | Psip1 | Ndufs8 | Basp1 | Dnajc2 | Rrp15 | |
| Snrpg | Med21 | Llph | Derl3 | Tead2 | 4921524J17Rik | Rps2 | |
| Fdx1 | Nme1 | Erdr1 | mt-Nd2 | Prmt1 | Gins4 | Tinf2 | |
| Glrx5 | Cdca8 | Atp5k | Cks1b | Esf1 | Naa38 | Lypla2 | |
| Alpl | Tsen34 | Rmdn3 | Eif3g | Banf1 | Pole3 | Ppm1g | |
| Elf3 | Oaf | Peg10 | Nop16 | Pin1 | Nucb2 | Dars | |
| Ndufa4 | Ccnb1 | Ccne1 | Itpa | Mta3 | Tomm7 | Ing1 | |
| Dynll2 | Ascl2 | Rps27l | Mat2a | Prim1 | Erh | Psmb2 | |
| Hsp25-ps1 | Lsm4 | Ezr | Gnl3 | Ppih | Rps8 | Fcf1 | |
| | Ahsa1 | Psmd7 | Pdcd5 | Eif3i | Samm50 | Rpl30 | |

Spiral Artery Trophpblast Giant Cells

| | | | | | | | |
|---|---|---|---|---|---|---|---|
| Car2 | Psg22 | Rgs17 | Psip1 | Eif3l | Got2 | Rps18 | Cct6a |
| Sct | Klhl13 | Mpzl2 | Tnfaip8 | Fscn1 | Hnrnpa2b1 | Actr3 | Nectin2 |
| 1500009L16Rik | Ldoc1 | Liph | Trap1a | Ehd1 | Prl7d1 | Anxa7 | Grhpr |
| Serpinb9e | Galk1 | Ddb1 | Tuba1c | Pramef12 | 1110008P14Rik | Cfl1 | Cct7 |
| Prl2a1 | Arpc1b | Irs3 | Cd82 | Eif1b | Rack1 | Gtf2c2 | Chordc1 |
| S100a6 | Anxa4 | Bex1 | Gjb5 | Mxd4 | Rps7 | Parva | Vma21 |
| Plac8 | Cdx2 | Lysmd2 | Serpine2 | Rap1a | Pdcd5 | Eef1g | Rpl39 |
| Serpinb9g | Tpm4 | Rpl22l1 | Tuba1a | Borcs7 | Cct4 | Cct2 | Ccnb1 |
| Prl6a1 | Anxa2 | Rhox5 | Txn1 | Torlaip2 | Mif | Rpl9 | Gm2000 |
| Lgals9 | Serpinb9b | 2310030G06Rik | Ralbp1 | Kit19 | Csrp1 | 0610007P141Rik | Snrpf |
| Prl7b1 | Derl3 | Pdlim2 | C430049B03Rik | Avpi1 | Cox5a | Nmrk1 | Aamp |
| Ada | Tfap2c | Nostrin | H2afz | Actg1 | Rpl27 | Eny2 | Smarcb1 |
| Aldh1a3 | Basp1 | Glrx5 | Pdcd4 | Cdkn2aipnl | Npm1 | Epop | Prelid1 |
| Serpinb6b | Rbbp7 | Tpm1 | Jup | Bex3 | Ppdpf | Ran | Pak1ip1 |
| Sri | Cald1 | Cnn2 | Morf4l2 | Dnajc8 | Ets2 | Krt18 | Hmbs |
| Fstl3 | Lasp1 | Grb2 | Pfn1 | Ubfd1 | Krk | Kat7 | Polr2j |
| Serpinb9d | Hmgn5 | Fblim1 | Actn1 | Cfap20 | Gga2 | Exosc8 | Calm3 |
| Prl2c5 | Spata21 | Upp1 | Aif1l | Zwint | Krt7 | Rpl23a | Ezr |
| H19 | Tbrg1 | Ppp1rl4b | Cdh5 | Rps4x | Ranbp1 | Rps8 | Rps3a1 |
| Aprt | Dusp9 | Cdkn1c | Eif4ebp1 | Mycbp | Rps4l | Rps3 | Elovl5 |
| Serpinb9c | Tmsb10 | Tfpi | Ercc1 | Ndufaf3 | Ywhab | Rrm2 | Rps17 |
| Ascl2 | Dynll2 | Fermt2 | Mvp | As3mt | Fkbp1a | Dtymk | Rps5 |
| Plac1 | Ctnnbip1 | Palm | Ndufa11 | Hat1 | Pdcl3 | Rpl10a | |
| Mt2 | Sin3b | Tubb5 | Ugp2 | Rps20 | Rps16 | Actr2 | |
| Fthl17a | Igfbp7 | S100a11 | Prmt5 | Myl6 | Gnai3 | Ola1 | |
| Tip53i11 | Mpzl1 | Krt8 | 1700086L19Rik | Pygl | Eif4e3 | Cklf | |
| Mrfap1 | Olr1 | Zyx | 1600025M17Rik | Rpp21 | Rpl12 | Cfdp1 | |
| Phactr1 | Mbnl3 | Alad | Arpc2 | Klhl22 | Tipin | Rps10 | |
| Tnfrsf9 | Myl12a | Fam162a | Abracl | Cetn3 | Arpc5 | Rpl36a | |
| Lgals1 | Nek6 | AA467197 | Vasp | Il2rg | Eif2s1 | Rps19 | |
| Pitrm1 | Sbsn | Rps27l | Gng12 | Plet1 | Chp1 | Snrpg | |
| Ncmap | Copz2 | Ncam1 | Sqstm1 | Gm9112 | Cep164 | Clqtnf6 | |
| | Eif2s2 | Dcakd | Tpm2 | Eif1a | Rpsa | Atpif1 | |

Spongiotrophoblasts

| | | | | | | | |
|---|---|---|---|---|---|---|---|
| Phlda2 | Cs | Pttg1 | Cops5 | Lsm8 | Impa2 | Drg1 | Mrto4 |
| Dio3 | Lgals1 | Trappc5 | Psmd12 | Gadd45g | 2010107E04Rik | Nae1 | Rnf128 |
| Dkkl1 | Hagh | Eif3g | Panx1 | Med7 | Ndufb5 | Hspa8 | Wdr77 |
| Hspb1 | Npm1 | Gpx4 | Dld | 2310033P09Rik | 0610007P14Rik | Dars | Pepd |
| Tmen14c | Tex30 | Gtf2h5 | Ppid | Atp11a | Gtf3c6 | Ubald2 | Ddx18 |
| Cidea | Mfge8 | Magoh | Dnajc2 | Skp1a | Dnajc19 | Hnrnpk | Lrrfip2 |
| Tfrc | Usp1 | Fam50a | Hspd1 | Eloc | Atp5k | Idh3a | Psmb7 |
| Batf3 | B3gnt7 | Cct3 | Hmgb2 | Nsmce2 | Tubb2a | Plekhf2 | Erdr1 |
| Sin3b | Mageh1 | Srsf3 | Uaca | Slc25a3 | Slirp | Vps35 | Rps28 |
| Prss8 | mt-Nd4 | Rfc4 | Wwtr1 | Gadd45b | Phb2 | Mrpl47 | Fnta |
| Ldoc1 | Emc8 | Eif1a | Psmd6 | Cfdp1 | Psmc1 | Birc5 | Rtn3 |
| Maoa | mt-Nd5 | Marcksl1 | Hnrnpc | H2afz | Folr1 | Unc50 | Idh3b |
| Cdkn1c | Commd4 | Serpinb9e | Mrps23 | Ppa1 | Bax | Dut | Elob |
| Las1l | Dnaja2 | Apoo | Nap1l1 | Atp5b | Rmdn3 | Cdc34 | Pfdn6 |
| Rhox6 | Tbca | Slc2a1 | Tead2 | Polr2e | G3bp1 | Nabp1 | Sugt1 |
| Tex19.1 | Ndufb2 | Vdac3 | Cd164 | Clns1a | Trim27 | Hadhb | Dstn |
| 2610528J11Rik | Tubb4b | Cox5a | Pparg | Dnajb6 | St13 | Aimp1 | Smarcb1 |
| Gkap1 | Sct | Ppp1r3g | Rpl22l1 | Rnf181 | Slc38a2 | Fus | Coq3 |
| Cldn7 | Ing2 | Cct5 | Rhox5 | Rnf4 | Dusp9 | Etfb | Igsf8 |
| Slc22a18 | Cd320 | Anxa4 | Psmd7 | Hdac1 | Cggbp1 | Hnrnpab | Tomm22 |
| Rhox9 | Hsd11b2 | Nsmce4a | Ndufa4 | Prpf19 | Ptma | Ndufb4 | Hmbs |
| Mrps6 | Vamp8 | C430049B03Rik | Ndufb6 | Nsmce1 | Chchd1 | Exosc8 | Cyc1 |
| Serpinb9g | Tbrg1 | Tmem147 | Tma7 | Gm11361 | Rpl18 | Rplp1 | Txnl1 |
| Aqp3 | mt-Nd2 | Pa2g4 | Med21 | mt-Rnr1 | Psmc6 | Cox7b | Fam104a |
| mt-Cytb | Gm9 | Tyms | Cox6b1 | Ncbp1 | Atp5c1 | Mrpl19 | Hn1 |
| Hsp25-ps1 | Slc38a1 | Eif4a1 | Tardbp | Blvra | Ero1l | Nsfl1c | Ctnna1 |
| Rdh12 | Rbbp7 | Snrpe | Uqcrc2 | Prpsap1 | Hspa9 | Timm17a | Ndufs8 |
| Krt18 | Atxn10 | Smu1 | Psma6 | Ube2e1 | Anapc15 | Pigp | Bsg |
| Pfdn1 | Hsp90aa1 | Tbcb | Larp7 | S100a16 | Rps8 | Ndufs1 | Gskip |
| Tulp1 | Calm1 | Basp1 | Ranbp1 | Serbp1 | Serpinb9d | Appbp2 | Cnih1 |
| Selenoh | Hspe1 | Fam90a1b | Mrpl4 | Rab10 | Cotl1 | Zwint | Rbm8a |
| Dynll2 | Fam136a | Nup85 | Suclg1 | Rala | Ash2l | Dusp11 | Gm2a |
| Glrx5 | Elf3 | Lonp2 | Pgrmc1 | Psmd13 | Arl6ip1 | Mcm2 | Eif3e |
| Slc16a1 | Prkd2 | Mrps22 | Mdh2 | Pmpca | Borcs7 | Set | Erh |
| Krt8 | mt-Co1 | Lyar | Rpl5 | Serpinb9b | Psmc2 | Scarb2 | Naa35 |
| Tmem150a | Ncl | Fermt2 | Ndufa5 | Ppa2 | Zcchc17 | Smc4 | Mrpl3 |
| Stx3 | Hadh | Srsf6 | Gucd1 | Hebp1 | Ncbp2 | Ywhaq | Map11c3b |
| Gjb2 | Cisd1 | Nxf7 | Car2 | Mrpl15 | Psmb1 | Cdca8 | Tcp1 |
| Nudt22 | Snrpg | Rad23b | Dnajc9 | Rrm2 | Prim1 | Hmgcl | Srsf10 |
| Mbnl3 | Syngr1 | Fkbp3 | Wdr18 | Ccnb1 | Thoc3 | Tra2a | Psma3 |
| Gm9112 | Chchd2 | Atp5o | Cox7c | Gpr137b | Nop58 | Npepl1 | Ndc1 |
| Cd9 | Ubqln1 | Cct8 | Ssb | Idh3g | Polr1d | Med28 | Mtch2 |
| Rbp1 | Fbxl19 | Snx5 | Ran | Srsf7 | Sap18 | H2afv | Psmd11 |
| Rps4l | Pphln1 | C1qbp | Emd | Slc25a4 | Gmfb | Sdhb | Rpl27 |
| Eif2s2 | Slc25a5 | Bglap3 | Hsp90ab1 | Gata2 | Lsm4 | Uqcrc1 | E2f5 |
| Ugp2 | Ccdc51 | Atp5f1 | Hnrnpa1 | Nhp2 | Rps5 | Nsrp1 | Pitpnb |
| Zfp655 | Mpdu1 | Chchd10 | AtpSa1 | Rars | Cdipt | Snrpf | |
| mt-Nd1 | Eif2s1 | Olr1 | Psmg2 | Snx6 | Usp14 | Snrpd2 | |
| Tdrp | Hspa14 | Cenph | Pdcd5 | Dpy30 | Psme3 | Rabif | |
| Urod | Prkcz | Uchl3 | Cacybp | Ube2c | Lamtor1 | Commd5 | |
| Hmgn5 | Taf1d | Cenpk | Lsr | Ahsa1 | Cycs | Smim11 | |
| Car4 | Mrpl16 | Pak1ip1 | Ttc4 | Peg10 | Ndufb8 | Cox4i1 | |
| Krt19 | 1700021F0 | Gm15536 | Cox7a2 | Eif3i | Imp4 | Cetn3 | |
| Rassf6 | 5Rik | Naa38 | Lsm6 | Mrpl55 | Mrps25 | Ruvbl2 | |
| Tfeb | Rap2c | Trpt1 | Stmn1 | Rfc5 | Nop16 | Strap | |
| Hbegf | Acvr2b | Psmc5 | Ccna2 | Cystm1 | Eif3d | Txn1 | |
| Rab9 | Irx3 | Got2 | Uchl5 | Ndufaf2 | Sae1 | Cyb5r3 | |
| Dnaja1 | Plac1 | Syce2 | Gadd45gip1 | Cox14 | Uqcrfs1 | Szrd1 | |
| Fh1 | Abhd5 | Atp5g3 | Epop | Usp39 | Ilf2 | Eef1g | |
| Atp6v0d1 | Serpine2 | Atp1b1 | Ndufb9 | Hat1 | Rad51 | Ndufs7 | |
| Impdh2 | Snrpd3 | Maea | Txndc9 | Lysmd2 | Psmc3 | Mrpl45 | |
| Ap1m2 | Prss36 | Psma1 | Slc38a4 | Psma7 | Hnrnpdl | Samm50 | |
| Sod2 | Perp | Ddx39 | Rbbp4 | Pole3 | Brix1 | Fdx1 | |
| Slc26a2 | Tmem109 | Tmem116 | Lgals1 | Renbp | Cox6c | Ndufv1 | |
| | Cct6a | Nasp | Psmf1 | Mrpl41 | Ddt | Snrpa1 | |
| | 3830417A13Rik | | | | | | |

Oligodendrocyte precursor cells (OPC)

| | | | | | | | |
|---|---|---|---|---|---|---|---|
| Spp1 | Mcm3 | S100a3 | Rassf4 | Adam9 | Irf1 | Col23a1 | Mmp2 |
| Ccnb1 | Pgcp | Creb5 | Nt5dc1 | Mns1 | Kif20b | Col4a5 | Plekhb1 |
| Pdgfra | Neu4 | Tram2 | Kif23 | Bcan | Tcn2 | Cd1d1 | Slc7a11 |
| Dcn | Emp3 | Serpinf1 | Troap | Zfp36l1 | Rnf180 | Pcdhga5 | Cenp1 |
| Rlbp1 | Slc6a20a | Enpp1 | Slc25a29 | Ssfa2 | Slc38a3 | Gal3st1 | Il18 |
| Slc6a13 | Igf2 | Tacc3 | Epn2 | Tnfrsfl1b | Lgals2 | Ddah2 | Alp1 |
| Inmt | Kif2c | Spry4 | Qpct | Gpr81 | 1700112E06Rik | Alx3 | Ccdc18 |
| Pnlip | Zcchc24 | Loxl3 | Gm19705 | Tmem146 | Neil3 | 4921530L18Rik | Fam35a |
| Lum | Mxra8 | Cyp1b1 | Timp4 | Kctd12b | 2900005J15Rik | Frmd8 | 2010317E24Rik |
| Cmbl | Ampd3 | Htra3 | Jun | Col9a3 | Clgn | Gpr146 | Fdxr |
| Pcolce | Ccnb2 | Ccl5 | Cxcl12 | Ostf1 | Cercam | Phldb2 | Med18 |
| Postn | Chst11 | Ezh2 | Col3a1 | D2Ertd750e | 6720463M24Rik | Itfg3 | Mtmr10 |
| Apod | Kif20a | Agbl2 | Rfx4 | Fbxo7 | LOC626693 | Trim45 | E130309F12Rik |
| Ednrb | Musk | Maml2 | Ppfibp1 | Clec1a | Ehd2 | Cdk4 | 1110031I02Rik |
| Scrg1 | S100b | Klhl5 | Cyr61 | Gpx7 | Thbs1 | Itga9 | Hells |
| Tmem45a | mt_AK131586 | Frmd7 | Zeb1 | Atp6v0e | Cd302 | Pryg | Trpv4 |
| Fam70b | Efemp1 | Ccl2 | Ppic | Cdk1 | Col15a1 | Cdk5rap2 | Cyp20a1 |
| Cspg4 | Gpc5 | Fam70a | Rhoc | Pcyox11 | Plekhg6 | Arhgap19 | Col4a1 |
| Cacng4 | Tmem176b | Abtb2 | Abhd2 | Caprin2 | Creb3l3 | 4930517E11Rik | Antxr1 |
| Fabp7 | Shc4 | Fkbp9 | Traf4 | Pabpc5 | Map3k8 | Rasl11a | Aldh1a1 |
| Pbk | Gm2a | Cenpe | Tspan4 | Fzd6 | Timp3 | Tuba1c | Gab1 |
| 1110015O18Rik | S100a1 | Slc2a12 | Cpxm1 | Gm5089 | Akap13 | Islr | 1300014I06Rik |
| Emid1 | Galnt3 | Slc22a8 | Sox10 | Cenpf | Arhgap29 | Prrx1 | 9930021D14Rik |
| Serping1 | S100a16 | Lad1 | E130114P18Rik | Mmp11 | Melk | Rrm2 | Tmem220 |
| Olig1 | C1qtnf6 | C1qtnf2 | Mfsd2a | Rasa3 | Antxr2 | Pars2 | Rhpn1 |
| Vtn | Afap1l2 | Ccnd1 | Lrp4 | Gsn | Bmp7 | Cftr | Tmem198b |
| Prc1 | Lbp | Lama1 | Fos | Gm9839 | Rab13 | Slc13a5 | Ebf1 |
| Fam180a | Cdkn2c | Smc4 | Tpx2 | Sal3 | Tsga14 | Lgals3bp | Ss18 |
| E130306D19Rik | Vipr2 | Adamtsl3 | Cenpi | 1810034E14Rik | Smpd2 | Cklf | E2f8 |
| Bgn | Chst5 | Vegfc | Lamc3 | Gpr37l1 | Abca6 | Col4a2 | Fam111a |
| Lmcd1 | Gpx8 | S100a6 | Mapk7 | Tril | Gatm | Vamp5 | Tgfbr3 |
| Col1a2 | Pdpn | Kank1 | Lama2 | Jam2 | Slitrk6 | Rassf8 | Sema5b |
| Spc25 | Lims2 | Irak4 | Fosb | Evi5l | Snx22 | Fam132a | Ifitm3 |
| Calcrl | Mavs | Sh3bp4 | Susd5 | Dna2 | Mpzl1 | Rftn2 | Gdpd2 |
| Itih5 | Aurka | Btd | Dpyd | Seipina3n | Prkcq | Dll1 | Cfh |
| Tmem100 | Emp1 | Mc5r | Uhrf1 | Cdc20 | 4933425H06Rik | Cald1 | Nnat |
| Adm | Olig2 | Rnf43 | Plekho2 | Sulf1 | Gprc5a | A430107O13Rik | D930014E17Rik |
| Tmem176a | Aox3 | Col1a1 | Tmc6 | P2rx7 | Pcca | Fam82a1 | Mcm9 |
| 0610040J01Rik | Myt1 | Bcas1 | Apobec3 | Map3k1 | Prelp | Tcirg1 | Gins2 |
| Pmel | Fignl1 | Plk1 | Fam114a1 | Dab2 | Gnb4 | Nusap1 | Slc1a5 |
| A930009A15Rik | Pcdhgc3 | Notch1 | Birc5 | Clqtnf7 | Cyp2j6 | Gpr182 | Ptgds |
| Cav1 | Gpsm2 | Angptl1 | B3gnt5 | Kif22 | Ctdsp1 | Serpind1 | Tnpo1 |
| Nupr1 | Mir568 | Cdca8 | Itgb8 | Xlr3b | Rab34 | Mcm7 | Ifitm2 |
| Gstm2 | Cd9 | Mc4r | Ston1 | Kif1Sa | Fzd9 | Sgk3 | Notch2 |
| Ckap2 | Fanci | Gpt2 | Kcnj10 | Zfp3612 | Msh6 | Lekr1 | Luzp2 |
| Spry1 | Fam64a | mt_AK143357 | 3632451O06Rik | S100a4 | Cep72 | Srpx2 | Murc |
| Top2a | Zic4 | Hapln3 | Socs3 | Scel | Otos | Gpld1 | |
| 1190002F15Rik | Cd40 | Lpo | Tmem144 | A330041J22Rik | Anxa2 | 1700013G23Rik | |
| Ube2c | Meox1 | Hps1 | Ptgfr | Plat | Ftsjd1 | Icam1 | |
| Ccl7 | Ect2 | Boll | Slc16a12 | Fam71f2 | Saa1 | Jam3 | |
| Cp | Rcn3 | Sema3d | Chaf1b | Smoc1 | Sh3tc2 | mt_AK159184 | |
| Vcan | Cyp2j9 | S100a13 | Dbi | Sox8 | Rnpepl1 | Cobll1 | |
| Ugdh | 1190002H23Rik | Nuf2 | Gfra1 | Hmgb2 | Atp1a2 | Traf1 | |
| Mdk | Wipf1 | Ggt5 | Cdca2 | Bmp6 | Pion | Mmd2 | |
| Gpr17 | Pold1 | Meis1 | Gpr82 | Pomt1 | Ppp1r14b | Sulf2 | |
| Tnfrsf1a | 1810010H24Rik | Cenpn | Nhsl1 | Orai1 | Myl12a | Cnn2 | |
| Ptprz1 | Cdc14a | Spsb4 | Zfp41 | Frrs1 | Ndc80 | Ror2 | |
| Cdc25c | Tgfa | Cks2 | Cyp4v3 | Shmt1 | mt_AK140174 | Rsu1 | |
| Pcdh15 | Tnr | Fkbp7 | Mtss1l | Plscr1 | AI854517 | 1700018G05Rik | |
| Ckap21 | Phxr4 | Pmp22 | Slc22a6 | Car8 | Matn4 | Rab31 | |
| Pdgfrl | Pllp | Cdca3 | Derl3 | Srebf1 | Foxc1 | Dynlt1c | |
| Lhfpl3 | Arhgap31 | Frk | Lima1 | Plekha2 | Vcam1 | Sfmbt2 | |
| Ogn | Kcnh8 | Kcnj16 | Eci1 | Txlna | Cpa4 | Nkiras2 | |
| Itih2 | Tbx18 | Ltbp1 | Selenbp1 | Epas1 | Mdfic | Wnt7a | |
| | Serpine2 | Cdo1 | Stk32a | 4933406J10Rik | Cspg5 | Mpzl2 | |

Astrocytes

| | | | | | | | |
|---|---|---|---|---|---|---|---|
| Gja1 | Gramd3 | Slc7a11 | Btd | Zfyve21 | Aldh6a1 | Alpl | Neu4 |
| Gjb6 | Slc7a10 | Phka1 | Gpld1 | Lgr4 | Pou3f4 | Glud1 | Ugt1a2 |
| Cldn10 | 3110082J24Rik | Id4 | Ccdc141 | Tmem176a | Clmn | Tsc22d3 | BCo13529 |
| F3 | Hsd3b7 | Agmo | ex_tRNA- | Sycp2 | Timp3 | Ccbl2 | Zfp783 |
| Slc1a3 | Mt1 | Fermt2 | Ala-GCG | Cpt1a | Slc6a20a | Tnfaip8 | Fjx1 |
| Slc39a12 | Bcan | Crot | Tom1l1 | Mettl11b | Mif4gd | Zfp438 | Rasl2-9-ps |
| Sdc4 | Appl2 | Elovl2 | Scrg1 | Loxl3 | Plscr2 | Hes1 | Suclg2 |
| Acsbg1 | Chi3l1 | Fkbp10 | Smpd2 | Abhd4 | Pnp | A130022J15Rik | Gdf10 |
| Mfge8 | Adhfe1 | Megf10 | Bdh2 | Papss2 | Btbd17 | Slc13a3 | Atp6v0e |
| Ntsr2 | Pxmp2 | AA387883 | Elovl5 | Pdgfrl | Pdk4 | Cklf | Csgalnact1 |
| Lcat | Tlr3 | Oaf | Cd38 | Retsat | Fzd2 | Egfr | 1700003M07Rik |
| Cml5 | Vcam1 | Il18 | Ttyh1 | Tcf7l2 | Slc7a2 | Ghr | Pyroxd2 |
| Aqp4 | Ctso | Pmp22 | Ccdc90a | Sema4b | Tubb2b | Slc25a35 | Efemp2 |
| Pla2g7 | Agxt2l1 | Fabp7 | Crlf3 | Rnase12 | Rapgef3 | Ephx2 | Afap1l2 |
| Ppap2b | AI464131 | Fam163a | Slc26a6 | Fgfr1 | Prkd1 | Rbp1 | Dbi |
| Ppp1r3c | Maob | Sat1 | Lxn | Igf2 | Adora2b | Pdlim5 | Gm10731 |
| S1pr1 | Rfx4 | Kirrel2 | Pcsk6 | Nat2 | Aox1 | Cdc42ep1 | 1190005I06Rik |
| Slc25a18 | Acat3 | Serhl | Paqr8 | Mir1192 | Hist2h3c1 | Qk | Abhd14b |
| Plcd4 | Mmd2 | Gstk1 | Luzp2 | Dcxr | Cyp7b1 | Farp1 | Trip6 |
| Chrdl1 | Ugt1a7a | Zfp36l2 | Egfl6 | Apln | Arsk | 2210417K05Rik | Lama2 |
| Fam107a | Gdpd2 | Arhgef26 | Fgd6 | Nrarp | Dhrs11 | Arap1 | Gm17660 |
| Dio2 | Bmpr1b | Slc4a4 | Hgf | S100a4 | S100a13 | Calm14 | Rin2 |
| Gpr37l1 | Prelp | Cyp4f13 | Cib1 | Sfxn5 | Hist1h2bq | Chst2 | Fndc4 |
| Mt2 | Pon2 | Emp2 | Hspb8 | Dok7 | Hist1h2br | Emx2 | Slc30a10 |
| Entpd2 | Tril | Gm973 | Acss1 | Plscr1 | Gng5 | Slc22a6 | Scg3 |
| Gstm1 | Gpc5 | Agt | Acsl6 | Dcn | Acsl3 | Parp3 | Abcd4 |
| Cbs | Nat8 | Lix1 | Pion | Ddo | Sult1a1 | Gm10052 | C230035I16Rik |
| Tst | C030037D09Rik | Upp1 | Notch2 | 1810014B01Rik | Maml2 | Ccdc18 | Ptplad2 |
| Prodh | Cyp4f14 | Naaa | Ppil6 | Nwd1 | Echdc2 | Tifa | Rasa2 |
| Slco1c1 | Nkain4 | Nfc2l2 | Tcn2 | Ugp2 | Tmem229a | Trim12a | Acadl |
| Gfap | Gm11627 | Steap3 | Renbp | Myo6 | c2_tRNA- | Serpine2 | Lrrc9 |
| Tlcd1 | Slc27a1 | Ptprz1 | Pax6 | Gpt | Ala-GCG | Mro | 1700040N02Rik |
| Mlc1 | Nat1 | Cd63 | Cyr61 | Cst3 | Notch1 | Vcl | Zfp521 |
| Apoe | Mertk | Cmtm5 | Gpam | Olfr287 | Slc12a4 | Per3 | Prkcd |
| C030018K13Rik | Fmo1 | Gabrg1 | Klf15 | Kctd14 | Agpat5 | Taf4b | Ranbp3l |
| Slc38a3 | 2900052N01Rik | Phkg1 | Swap70 | Zbtb20 | Rlbp1 | Il13ra1 | Npc1 |
| Aldoc | Cth | Gas1 | Slc6a11 | Ddhd1 | LOC433374 | 1190002H23Rik | Hif3a |
| Timp4 | Tmem100 | Selenbp1 | Lgals4 | Znrf3 | Kctd12b | Gypc | Pfkfb1 |
| Cyp2d22 | Cideb | Gpx8 | Psd2 | Olfml1 | Eci1 | Kcnj13 | Fcgr2b |
| Slc15a2 | Cml1 | Soat1 | Pnpla7 | Rmst | Tex11 | Gabrb1 | Rdm1 |
| Htra1 | Efemp1 | S100a1 | Sall3 | Tmcm51 | Lmcd1 | Cmtm3 | Mmp14 |
| Atp13a4 | Mdk | Thrsp | Myo10 | Hsd11b1 | Cbr3 | Itga7 | Grtp1 |
| Atp1a2 | Kcnj16 | A330048O09Rik | Elmod3 | Rdh5 | Zic5 | Angptl1 | Wnt7b |
| Prdx6 | Daam2 | Sc4mol | Hist1h2bc | Eya1 | Calr4 | Stk17b | Trp53bp2 |
| 2010002N04Rik | Scara3 | Rfx2 | Smox | Odf3l1 | Lhx2 | Hacl1 | C2 |
| Fgfr3 | Mfsd2a | Phgdh | Nde1 | Kank1 | Atp1b2 | Olfr288 | Lgals3bp |
| Pdpn | 1700084C01Rik | Hopx | A330076C08Rik | Paqr6 | Sox21 | Fam181b | |
| Sox9 | Rftn2 | Naprt1 | 2610034M16Rik | Utp14b | Gjb2 | Ccdc77 | |
| Fxyd1 | Prex2 | Ndrg2 | Gm13031 | Histlh4h | Dera | D630033O11Rik | |
| Itih3 | Dhrs3 | Acaa2 | Enho | Lpcat3 | Hsdl2 | Phxr4 | |
| Fam176a | Grm3 | Slc1a2 | Tnfsf13 | Aldh1a2 | Lpin3 | Nek3 | |
| Cyp4f15 | 1700019G17Rik | B230209K01Rik | Plxnb1 | Lum | Vgll4 | 1700084J12Rik | |
| Gldc | Hepacam | S100a16 | Cdkn2c | A2m | Zcchc24 | Asrgl1 | |
| Cml3 | Pgcp | Pbxip1 | Gem | Rpe65 | Slc22a4 | Gprc5d | |
| Ndp | Clu | Spata17 | Tmem176b | Rcn3 | Kcnj10 | Decr1 | |
| Cyp2j9 | Smpdl3a | Lpar4 | Nudt7 | Gna13 | Vav3 | Lonrf3 | |
| Slc14a1 | Fam20a | Gpr56 | E030003E18Rik | Cyp2j6 | Gli3 | Rnf182 | |
| E130114P18Rik | Gm5083 | Aass | Cnn3 | Fpgs | Akt2 | Mmgt2 | |
| Pdlim4 | Abhd3 | Hadh | 4932438H23Rik | Plod1 | Eps8 | Paqr7 | |
| Aldhi1l1 | Ednrb | Acot11 | Lrp4 | Fgfr2 | Nfia | Hapln1 | |
| Mgst1 | St3gal4 | Pax6os1 | Id3 | Dock1 | Tsc22d4 | Cox6b2 | |
| Dbx2 | Rarres2 | Ttpa | Aqp9 | Frrs1 | Lrrc51 | Sohlh2 | |
| Ezr | Glul | Gstt3 | Hist1h4i | Fads2 | Grhl1 | Nphp3 | |
| Slc9a3r1 | Fam198a | Cdh19 | Tdo2 | Sepp1 | Tnfrsf19 | Idh2 | |
| | Gm5089 | Nr1h3 | Gstm5 | Trp63 | Adrbk2 | Btg1 | |
| | | | Slcolb2 | | 2810055G20Rik | | |

Cortical Neurons

| | | | | | | | |
|---|---|---|---|---|---|---|---|
| Nos1 | Scrt2 | Neurod2 | Serpini1 | Nedd4l | Gstm7 | Elavl4 | Cdk2apl |
| Fam84a | Cdh4 | Srrm4 | Ttc28 | Faml14a2 | Emx1 | Scg5 | Cplx2 |
| Unc5d | Slc17a6 | Adgrl2 | Epha5 | Cux1 | Tmcm108 | Scenl | Efnb2 |
| Rnd2 | Osbpl6 | Jarid2 | Ankrd6 | Mta2 | Dbn1 | Ptprs | Klhdc2 |
| Pou3f2 | Sema3c | Pou3f3 | Tmcm158 | Acly | Mytl1 | Midn | Ccng2 |
| Pdzm3 | Kif21b | Cttnbp2 | Plxna4 | Baz2b | Cul1 | Kdm2b | Parp6 |
| Hs3st1 | Wnt7b | X6330403K07Rik | Nfasc | Phf21b | H1f0 | Laptm4a | Nipsnap1 |
| Sstr2 | Tbr1 | Nav2 | F2r | Phip | Kif21a | Fam49a | Tax1bp3 |
| Pcp4 | Chga | Pantr1 | Fmnl2 | Tmeff1 | Ilf2 | Acin1 | Ezr |
| Meis2 | Tenm4 | Lrpap1 | Cbfa2t2 | Ddah2 | Rpf1 | G3bp2 | Nol4 |
| Lrrc16b | Lmo1 | Trim2 | Lzts1 | Grina | Ing4 | Mdk | Elavl2 |
| Plekhf2 | Tsc22d1 | Nek6 | Sorbs2 | Smim18 | Hist3h2a | Sbk1 | Arhgef2 |
| Sorl1 | Igfbpl1 | Ldhb | Frmd4a | Rbfox1 | Bcl7a | Auts2 | Nsg2 |
| Ppp2r2b | Nrn1 | Lhx2 | Plxna2 | Sncaip | Hivep3 | Kdm5b | Pbx1 |
| Trim9 | Wbscr17 | Tagln3 | Foxg1 | Lrp8 | Hbb.bs | Ap3s1 | 43346 |
| Pou3f1 | Itpk1 | Mn1 | Cdkn1b | Avl9 | Gdap1l1 | Basp1 | Zfp462 |
| Frmd4b | Sox5 | Vopp1 | Luzp2 | Nfix | Fam107b | Tmcm57 | |
| Mllt3 | Prex1 | Gm17750 | Dpy19I1 | Tnrc18 | Podxl2 | Peli1 | |
| Plcb1 | Rcor2 | Nfib | Rbfox3 | Znrf2 | Setbp1 | Cux2 | |
| Ppp2r1b | Kctd4 | Neurod6 | Cd24a | Adgrg1 | Wbp1 | Ttc9b | |
| Lsamp | Cited2 | Rasgef1b | Cd1d1 | Abracl | Ip6k2 | Rundc3a | |
| Enc1 | Epha3 | Hs6st2 | Cyth2 | Mpped1 | Igsf3 | Mpped2 | |
| Robo2 | Palmd | Insm1 | Negr1 | Gria2 | Gm14964 | Mkrn1 | |
| | Bcar1 | Tmem178 | Hist3h2ba | Zbtb18 | Nrp1 | Akap9 | |

RadialGlia-Id3

| | | | | | | | |
|---|---|---|---|---|---|---|---|
| Id3 | Hey1 | Efcab1 | Add3 | Morn2 | Slc25a25 | Pex7 | X2810417H13Rik |
| Id1 | Aldoc | Nes | Lrp4 | Naf1 | Pmp22 | Galk1 | Ext1 |
| Foxj1 | Anxa2 | Mest | Ifitm3 | Crip1 | B9d1 | Hsd17b7 | Tanc1 |
| Mt1 | Atp1b2 | Slc6a11 | Tspan15 | Grb10 | Purb | Anxa5 | Lhfp |
| Mt2 | Ncan | Glul | Slc27a1 | Itm2c | Ctso | Ift22 | Amot |
| Pla2g7 | Atp1a2 | Fam181b | Glud1 | Sparc | Axl | Sgcb | F3 |
| Hes5 | Cybrd1 | Camk2d | Timp3 | Mmd2 | Dhcr24 | 43358 | Pmf1 |
| Hes1 | Tmem107 | Zfp36l2 | Hopx | Mcm3 | Tpp1 | Tmem218 | Stat3 |
| Mia | Lgals1 | Gja1 | Cav2 | Acyp2 | Stxbp6 | Slc1a2 | Ppp1r1a |
| Egr1 | Slc14a2 | X2810459M11Rik | Arl4a | Adcyap1r1 | Rasa3 | Rbp1 | Gprc5b |
| Metrn | Rhoq | Spry2 | Chpt1 | S100a13 | Cbfb | Arhgef26 | Dhfr |
| Fos | Tlcd1 | Vim | Fhl1 | Eif4ebp1 | Pacsin2 | Dnajc15 | Lyrm5 |
| Tmcm47 | Rhoc | Acadl | Tst | Irs1 | Gcsh | Pmm1 | Cdk2 |
| Ednrb | Sox9 | Igfbp2 | Plpp3 | Cib1 | Parva | Cfap36 | Nfkbia |
| Tppp3 | Ccnd1 | Ckb | Spa17 | Afap1l2 | Zeb1 | Etfa | Cntln |
| Clu | X1500015O10Rik | Paqr8 | Tom1l143352 | Ttyh3 | Nkain4 | Pid1 | Gas1 |
| Serpine2 | Bhlhe40 | Gng5 | Msn | Notch2 | Snx5 | Ctdsp1 | Pfn1 |
| Riiad1 | Zfp36l1 | Hspa2 | Pttg1 | S100a6 | Ormdl2 | Eci1 | Prdx1 |
| Gfap | Ddit4l | Lrig1 | Ninj1 | X2610301B20Rik | Adgrv1 | Plxnb1 | Golph3 |
| Sparcl1 | Nim1k | Erf | Fkbp9 | Magt1 | Stard4 | Klf6 | Cystm1 |
| Apoe | Nme5 | Zic5 | Ctsc | Itgb5 | Car2 | X1500009L16Rik | Kcnip3 |
| Slc1a3 | Lfng | X1810037I17Rik | Rrbp1 | Kbtbdl1 | Sox21 | Emc7 | Prdx4 |
| Nlrx1 | Tagln2 | Bc12 | Prkcdbp | S100a1 | S1pr1 | Dennd2a | Rad23a |
| Selm | Mfge8 | Ier2 | Gnai2 | Mif4gd | Slc12a4 | Zdhhc21 | Tram1 |
| Ttyh1 | Stom | Vcam1 | Nr3c1 | Tnfaip8 | Hacd1 | Plce1 | Dclk1 |
| Gstm1 | Pbxip1 | Ptn | Ldha | Pcx | Cd9 | Oat | Hspa5 |
| Lxn | Emp1 | Nkd1 | Slc38a3 | Dnajc3 | Wwp1 | Myo10 | Gm2a |
| Cyr61 | Mpp6 | Trim47 | Zcchc24 | Dag1 | Jun | Phyhip1 | Smo |
| Fbxo2 | Pdpn | Ptprz1 | Znrf3 | Rgs20 | Klhl13 | Maml2 | Spcs3 |
| Mlc1 | S100a16 | Krcc1 | Akr1b10 | Tapbp | Gabrb1 | Irs2 | AI854517 |
| Enkur | Tspan33 | Scd2 | Hadh | Hmgcs1 | Msi2 | Msmo1 | Flna |
| Mlf1 | Aldh1l1 | Tnfrsf19 | Myo6 | Nudt4 | B230118H07Rik | Mras | Csrp1 |
| Mgst1 | Fam212b | Zfp36 | Kcnj10 | Mlec | Eef0kmt | Mtss1l | Gpt2 |
| Slc9a3r1 | Fzd9 | Idi1 | Acadm | Degs1 | Nr2c2ap | Asrgl1 | Ift74 |
| Bcan | Pdlim5 | Serpinh1 | Psph | Abhd4 | Dpcd | Fam195a | Syt11 |
| Fabp7 | Eepd1 | Ntrk2 | Psat1 | Sp3os | Il6st | Socs2 | Clic1 |
| Dbi | Ier3 | Suclg2 | Prrx1 | Sash1 | Rgcc | Fads1 | Il18 |
| Emp2 | Fbln2 | Metrn1 | Tns3 | Fjx1 | Rnft1 | Trip6 | My112a |
| Ppp1r3c | Junb | Rgma | Slc39a1 | Uhrf1 | Rasl11a | Rexo2 | Scrg1 |
| Igfbp5 | Pea15a | Rcn1 | Itgav | Slc15a2 | Ak3 | Ptgfrn | Nphp1 |
| Wls | Kcne1l | Axin2 | Gm5617 | Cenpw | Echdc1 | Sri | Pr0m1 |
| Tpbg | Etv4 | Klf9 | Ccpg10s | X1110004 | Nr2f6 | Nfc212 | Ctnna1 |
| Fgfr3 | Ramp1 | Klf15 | Notch1 | E09Rik | Vamp3 | X2310022B05Rik | Pde4b |
| Hepacam | Sfxn5 | Npas3 | Prr18 | Cebpb | Arhgef40 | Snx3 | Lig1 |
| Aqp4 | Egfr | Sat1 | Cbs | Tspan12 | Ifngr1 | Thbs3 | Itgb8 |
| Olig1 | Klf4 | Chst2 | Rest | Trib1 | Phxr4 | Pcdh10 | Sox8 |
| Tnc | Gpx8 | Paqr4 | Anxa6 | Pcgf5 | Tm7sf2 | E10f1 | |
| Mt3 | Cpne2 | Cd63 | Insig1 | Pnp | Mvk | Tctex1d2 | |
| Slc4a4 | Chchd10 | Spry1 | Nrarp | Fam120a | Dnajc24 | Fgfr2 | |
| Gng12 | Ndrg2 | Dkk3 | Emc2 | Gmnn | Hsdl2 | 43345 | |
| Pacrg | Rmst | Bmpr1a | Thrsp | Polr3h | Bola3 | Bet1 | |
| Rspo3 | Nebl | Epdr1 | Efemp2 | Creb5 | Wwtr1 | Spsb4 | |
| Phgdh | Jam2 | Yap1 | Acot1 | Pygb | TraB | Lss | |
| Tril | Acsbg1 | Adamts1 | Bph1 | Trim9 | Spata24 | Phlda3 | |
| Qk | Pon2 | Mns1 | Nr4a1 | Ppargc1a | Bak1 | E2f5 | |
| Ccdc80 | Fosb | Aldoa | Ppic | Grm5 | Tspan7 | Nrcam | |
| Aard | Smpd13a | Ccnd2 | Cxxc5 | Rab31 | Lppos | Ddah1 | |
| Plat | Fat1 | Slc1a4 | Il11ra1 | Grhpr | Nab2 | Klhdc8b | |
| Olig2 | Sema6a | Nog | Gins2 | Btg2 | Mcee | Plin3 | |
| Rfx4 | Gdpd2 | S100a11 | Rorb | Galc | Chsy1 | Klf10 | |
| Cmtm5 | Tsc22d4 | Itga6 | Sox2 | Tjp1 | Dusp6 | Klf3 | |
| Id4 | Sall3 | Fgfbp3 | Rab13 | Cnp | Mid1ip1 | Gltp | |
| Socs3 | Gsta4 | Dusp1 | Nacc2 | Donson | Cetn2 | Ccdc8 | |
| Scd1 | Cspg5 | X3110082J24Rik | Ung | Cst3 | Dtd2 | Specc1 | |
| | Neat1 | X1700088E04Rik | | Hspa4l | Trps1 | X4933434E20Rik | |
| | | | | Cln5 | | | |

RadialGlia-Gdf10

| | | | | | | | |
|---|---|---|---|---|---|---|---|
| Gdf10 | Ass1 | Pdpn | Arhgef26 | Gmnn | Lig1 | Rfc1 | Msi2 |
| Id3 | Htra1 | Dkk3 | Rcn1 | Pdcd4 | Prps2 | Glo1 | Tyms |
| Tesc | X2810459M11Rik | Col9a3 | Nova1 | Cd164 | Gstm5 | Tpx2 | Spg20 |
| Thrsp | Bcl2l12 | Mgst1 | Appl2 | Maml2 | Naa50 | Atxn7 | Fut9 |
| Tnfrsf19 | Gja1 | Lrp4 | Mki67 | Scrg1 | Sypl | Cenpw | Prox1 |
| Frzb | E1301114P18Rik | Foxo1 | Phxr4 | Kcnmb4 | Krcc1 | Ddah1 | Pmp22 |
| Id1 | Nkd1 | Dmd | Anxa6 | Ccna2 | Eci2 | Prox1os | Ccdc34 |
| Sdpr | Ninj1 | Entpd2 | Nr2f6 | Kbtbd11 | Jam2 | Tor1b | Snta1 |
| Emid1 | Enpp2 | Dmrt3 | Gli3 | Lap3 | Cisd3 | Asah1 | Cdv3 |
| E330013P04Rik | Fzd1 | Chst2 | Tgif1 | Knstrn | Fezf2 | Ndufc2 | Tmem256 |
| Hspb8 | Selm | Gpx8 | Pygb | Gng5 | Lhfpl2 | Bmpr1a | Ss18 |
| Pdlim3 | Hadh | Tsc22d4 | Tspan15 | Chpt1 | Mcm5 | Crip2 | Aamdc |
| Dcn | Psph | Isoc1 | Sdc2 | Snx5 | Nadk | Cpne3 | 43345 |
| Gfap | Sfxn5 | Fkbp10 | Tspan12 | 43351 | Tjp1 | Lysmd2 | Sox6 |
| X1500015O100Rik | Aard | X1110015O18Rik | Fat1 | Slit2 | Cxxc5 | Sat2 | Arhgap5 |
| Mt2 | Lrrc1 | Gng12 | Zfp36l2 | Itgb8 | Prom1 | Abhd4 | Paics |
| Lef1 | Dbi | Epdr1 | Hells | Mcm3 | Pacsin3 | Fam120a | Snap23 |
| Rmst | Fras1 | Cpne2 | Hmgb2 | Prdx4 | Pank1 | Rcn3 | Scd2 |
| Gas1 | Slc9a3r1 | Ptgfrn | Cdca8 | Litaf | Dennd2a | Cks1b | Ctdsp1 |
| Tst | Ltbp1 | Mt3 | Cst3 | Ctdsp2 | Rdm1 | Kpna2 | Gsr |
| Mgll | Dmrta2os | Zic1 | Aif1l | Kcnip1 | Usp1 | Evi5 | Fkbp9 |
| Zic5 | Notch1 | Lmcd1 | Itga6 | Hn1l | Cmc2 | Pmf1 | X4933431E20Rik |
| Sp5 | Lhfp | Notch2 | Lockd | Gcsh | Nit2 | Dpysl4 | Atp1b1 |
| Hopx | Emx2 | Id4 | Gstm1 | Hs2st1 | Adgrb1 | Ifitm2 | Exosc5 |
| Prex2 | Bcl2 | Msn | Acot1 | Cdk1 | Nme4 | Bach2 | Mettl1 |
| Eya1 | Axin2 | Mlc1 | Ube2c | Slc1a4 | Echdc1 | Slc35a4 | Atp1a1 |
| X0610040J01Rik | Etv4 | Qk | Pttg1 | Dhcr24 | Apoe | Kcne1l | Syce2 |
| Cav1 | Sez6l | Smco4 | Lix1 | Arl4a | Mcm6 | Cdol | Ost4 |
| Mt1 | Efcab1 | Eepd1 | Btg3 | Dhfr | Smc2 | Siva1 | Actn1 |
| Adamts19 | Fos | Myl9 | Otx1 | Shisa4 | Dclk1 | Pcna | Rangrf |
| Wnt8b | Mro | Cdkn2c | Cbfb | Tmem107 | Dtymk | Efemp2 | Hmgn3 |
| Nme7 | Tnc | Tspan7 | Pnp | Pcx | Jam3 | Cntln | Nrarp |
| Crip1 | Rhoc | Cd9 | Tgif2 | Ldha | Pax6 | X2310022B05Rik | Carnmt1 |
| Zfp36l1 | Rfx4 | Gabra4 | Cks2 | Slc39a1 | Paqr4 | Acadm | Hmbs |
| Cyp1b1 | Rgma | Dtl | Pbk | Serpinh1 | Stard4 | Ier2 | Rnft1 |
| Lhx9 | Grb10 | Gnai2 | Rpa2 | Tcf19 | Elavl1 | Cdc42se1 | Syt11 |
| Vim | Ung | Plpp3 | Limd1 | Bola3 | Vcan | Adrbk2 | Fuz |
| Rgs20 | Atp1a2 | Cenpf | Idi1 | Nde1 | Hist1h1e | Mvk | Tspan18 |
| Hes5 | St3gal4 | Klf9 | Cyba | E2f5 | Tulp3 | Rragd | Fam96a |
| Tpbg | X2700046A07Rik | Fam167a | Top2a | Camk2d | Mcee | D8Ertd82e | Dennd5a |
| Slc1a2 | Fbln2 | Gldc | Sesn3 | Cdk2 | Nudt5 | Nudt4 | Nudcd2 |
| Aldoc | Veph1 | Paqr8 | Csrp1 | Ccnb2 | Ptprg | Csad | Dnph1 |
| Slc1a3 | Tmem132c | Rftn2 | Tanc1 | S100a11 | Hist1h2ap | Purb | Ybx3 |
| Psat1 | Dmrta2 | Stxbp6 | Erf | Tmem97 | Decr1 | Rpl22l1 | Specc1 |
| Ttyh1 | Col2a1 | X2310009B15Rik | Sox8 | Rab11fip2 | Higd1a | Fjx1 | Tpi1 |
| Hes1 | Emp2 | Gins2 | Tex9 | Eef1d | Ift74 | Mpp6 | Akr7a5 |
| Tspan33 | Nim1k | Uhrf1 | Map3k1 | Mcm4 | Lsm2 | Bcl7c | |
| Cpne8 | Loxl1 | Ephb1 | Fignl1 | Suclg2 | Ldlrad3 | Stx4a | |
| Hepacam | Pbxip1 | Clu | Sirpa | Gem | Cachd1 | Mgat1 | |
| Sox9 | Mfge8 | Lrrc4c | Spc24 | Ehbp1 | Ppp1r1a | 43358 | |
| Vcam1 | Rest | Gsap | Dnajc1 | Insig1 | Hist1h4i | X2810004N23Rik | |
| Ccnd1 | Trip6 | X2810417H13Rik | Ephb3 | Pdk3 | Acadl | X1500011K16Rik | |
| Tmem47 | Gabrb1 | Cdca3 | Atp1b2 | Amot | Mcm2 | Anp32b | |
| Glud1 | Fgfr3 | Socs2 | Mif4gd | Smo | Nacc2 | Rpa1 | |
| Sned1 | Pon2 | Adcyap1r1 | Hey1 | A730017C20Rik | Prdx1 | Spred1 | |
| Ccdc80 | Tns3 | Ptn | Klhl5 | Vamp3 | Fxyd6 | Hspa4l | |
| Fbxo2 | Tgfb2 | Yap1 | Birc5 | Ramp2 | Nr2e1 | Crot | |
| Lfng | Fam49b | Cbs | Sapcd2 | Arhgef40 | Itgb3bP | Tmem167 | |
| Tfap2c | Prkcdbp | Sparc | Tead2 | Eps15 | Ckap2 | Echdc2 | |
| Ndrg2 | Cspg5 | Cenpm | Eci1 | Wwtr1 | Vldlr | Cald1 | |
| Cthrc1 | Zcchc24 | Cyr61 | Chd7 | Rnf26 | Tipin | Lhx2 | |
| Cav2 | Slc27a1 | Prdx6 | Npas3 | Vgll4 | Homer2 | Nek6 | |
| Mmd2 | Sash1 | Vat1l | Cenpa | Rexo2 | Kctd12 | Lyrm5 | |
| Phgdh | Gas6 | Sox2 | Hrsp12 | Btg1 | Dag1 | Toporsos | |
| | Adgrv1 | Ttyh3 | Klf4 | Cdon | Rpe | Arl6 | |

RadialGlia-Neurog2

| | | | | | | | |
|---|---|---|---|---|---|---|---|
| Neurog2 | Kif26b | Wasf2 | Dnajb2 | Echdc1 | Asah1 | Hyal2 | Ndufaf7 |
| Eomes | Tmem98 | Eci1 | Asnsd1 | Elavl1 | B230354K17Rik | Nrn1 | Gm8730 |
| Gadd45g | Fam53b | Mmp14 | Zbed3 | Akr7a5 | Acadvl | Shmt2 | Dexi |
| Rhbdl3 | Dhx32 | Ckb | Vps37b | Ift22 | Cnih4 | Zfp62 | Pno1 |
| Ptgds | Abcd2 | Gadd45gip1 | Fubp3 | Ctnnb1 | Yif1a | Svip | Gspt1 |
| Btbd17 | Lzts1 | Ddah1 | Dcaf8 | Azi2 | Ift52 | Ubxn2a | Fxn |
| Snhg18 | Dll3 | Glo1 | Tbrg1 | Ece2 | Srsf6 | Rad23a | Snhg6 |
| Lima1 | Aifl1 | Ccs | Ufm1 | Pmepa1 | Hibadh | Golim4 | Ccdc86 |
| Tfap2c | Cbs | Ift74 | Wscd1 | Bphl | Foxp4 | Scrn1 | Bola3 |
| Mfng | X1500015O10Rik | Slc25a5 | Lta4h | Fundc2 | Gnpda2 | Vik3 | Kti12 |
| Btg2 | Gpx8 | Sfxn5 | Idh2 | RP23.207N5.2 | Cpne3 | Urod | Pou2f1 |
| Myo10 | Cmc1 | B230118H07Rik | Gstm5 | Paics | Lamp2 | Taf10 | Mrpl24 |
| Csrp1 | Slc1a2 | Pam | Sema5b | Rbpj | Itgb3bp | Pdcd4 | Rit1 |
| Tead2 | BCl2l12 | Lzts2 | Hadh | Rangrf | Rcor2 | Rbfox3 | Lztfl1 |
| Pax6 | Rnaseh2b | Hmgn2 | Ftsj3 | Rpl22l1 | Cplx2 | Mphosph10 | X1810058I24Rik |
| Celsr1 | Mcm2 | Ddr1 | Pyurf | Ptbp1 | Cadm3 | Emg1 | Swt1 |
| Gm29260 | Ezr | Ninj1 | Eci2 | Nedd4 | Ankrd6 | Smarcad1 | Eif3i |
| Chd7 | Gng5 | Srek1ip1 | Paqr8 | Aco1 | Myl12a | Rrp15 | Spata2 |
| Acads | Tank | Adk | Fam96a | Flna | Lman2 | Ldha | Tef |
| Heg1 | Apool | Snx5 | Atf5 | Nkain4 | Cnpy2 | Ppib | Vamp3 |
| Dll1 | Spsb4 | Acot1 | Rps18.ps3 | Rprm | Mrpl17 | Cdk4 | Ift43 |
| Gamt | Hrsp12 | Zfand1 | Cdca7 | AI854517 | Trp53 | X1500011K16Rik | Guf1 |
| Kcne1l | Cd63 | X2610301B20Rik | Rexo2 | Polr3k | Mrps14 | Tmed1 | Gm10020 |
| Tox3 | Ccdc136 | Serpinh1 | X2810004N23Rik | Hsd17b4 | Fars2 | Cdk5rap3 | X2310011J03Rik |
| Rcn1 | Ddit4 | Cib1 | Prdx1 | Trap1 | Serinc2 | Acly | Setbp1 |
| Gfap | Grb10 | Fbln1 | Efs | Mcee | Prdx3 | Lyrm4 | Rnf13 |
| Igfbp5 | Pttg1 | Syne2 | Golph31 | Npc2 | Fam162a | Slc48a1 | Mccc1 |
| Hes6 | Nr2e1 | Nrg1 | Echs1 | D10Jhu81e | Atp5g2 | Mt2 | Akr1b3 |
| Efhd2 | Tmem218 | Ncald | Ormdl2 | Mettl1 | Sp3os | X1110012119Rik | Hspe1 |
| Inppl1 | Btg3 | Elavl2 | Exosc3 | Dazap2 | Mcttl5 | Fam174b | Ralgds |
| Lrrn3 | Zeb1 | Phgdh | Ccdc58 | Ino80b | Clic4 | X1810037I17Rik | Hmgn5 |
| Sfrp1 | Eef1d | Ly6e | Anp32b | Rbbp9 | Twf1 | Hnrnpf | Immp1l |
| Nme4 | Sstr2 | Insm1 | Cul1 | Prdx6 | Lap3 | Tpm4 | Carnmt1 |
| Sox21 | Thrsp | Abca1 | Sox6 | Elp4 | Creb5 | Mt1 | Iscu |
| Loxl1 | Sema5a | Slc1a3 | Hdac1 | H1f0 | Emx1 | Acvr2b | Isca2 |
| Fam210b | Gas1 | Ttc8 | Tmem33 | Exosc5 | Rrs1 | Gcsh | Tspan3 |
| Dbi | Slco1c1 | Phyh | Limd1 | Sipa1l1 | Cdkn2c | Itf57 | Gkap1 |
| Tgif2 | Rcn3 | Ccdc167 | Tor1aip1 | Sesn1 | Rps27l | X2310039H08Rik | Actl6a |
| Ccnd2 | Ctnna1 | Dnajc15 | Por | Gm14305 | Ebpl | Rpe | Pdia6 |
| Vim | F2r | Lyrm5 | Adcyap1r1 | Pbdc1 | Timm21 | Zbtb38 | Ppie |
| Mfap4 | Zfp703 | Smpd2 | Cyba | Wdr61 | Nsmce4a | Crnkl1 | Sod2 |
| Mdk | Mdga1 | Litaf | Hadha | Adgra3 | Dhx40 | Aamdc | Odc1 |
| Notch1 | Inhbb | Nudt5 | Tead1 | Pabpc1 | Mmd2 | Gnpat | Fuca1 |
| Gem | Pnpla2 | Krcc1 | Calu | Llgl1 | Rhoc | Pfkl | Polr3c |
| Magi1 | Zfp36l1 | Scp2 | Ndufc2 | Clic1 | Ppp2r3d | Gm10073 | Med9 |
| Coro1c | Stifu | Ube2g2 | Etfa | X2210016F16Rik | Spire1 | Mybbp1a | Pex9 |
| Mfap2 | Smco4 | Bet1 | Dync2li1 | Draxin | H2afv | Capn2 | |
| E130114P18Rik | Rab8b | Trappc6a | Tmed10 | Ginm1 | Mrpl54 | Eif1b | |
| Dleu7 | Dmrta2 | Tsc22d4 | Snapin | Ddx52 | Tle1 | Ntrk2 | |
| Ascl1 | Ndrg2 | Actr3b | Lrp8 | Msi2 | Tpcn1 | Pgam1 | |
| Igdcc4 | Cdk2ap1 | Dnajc24 | Hdhd2 | Zfp219 | Igbp1 | Josd2 | |
| Tmem132b | Ehbp1 | Sdc3 | Cdk6 | Ppp2r3c | Ikzf5 | Trpc4ap | |
| Myo6 | Echdc2 | Sox2 | Ss18 | Rcn2 | Sec23b | Ctsz | |
| Uaca | Egr1 | Fezf2 | Ctage5 | Arl6ip6 | Chrac1 | Ubxn4 | |
| Slc30a10 | Hs3st1 | Gtf3c6 | Pcbd2 | Tmed4 | Smim20 | Leng1 | |
| Gm11627 | Msn | Emid1 | Fam58b | Stx4a | Gpi1 | Tmem230 | |
| Pdlim4 | Hmg20b | Pcmtd2 | Qars | Klf3 | Pts | Tmem178 | |
| Zhx2 | Cbfa2t2 | Aldh6a1 | Tfdp2 | Ivd | Plagl1 | Sat2 | |
| Jam3 | Rgs3 | Prmt8 | Aldh7a1 | Fgd4 | Rcbtb2 | Cd320 | |
| Zfp423 | Elavl4 | Smim11 | Kat6b | Bbx | Mrpl10 | Dennd5a | |
| Cd164 | Aldh2 | Kdm7a | Nit2 | Ssbp1 | Pgap2 | Ost4 | |
| Pgpep1 | Chn2 | Qsox1 | Tcf3 | Hadhb | Zmiz1 | Nabp2 | |
| Dhrs4 | Rab13 | Nrarp | Adgrg1 | X2810006K23Rik | Slc35b2 | Nudcd2 | |
| Igsf8 | Fdx1 | Pex7 | Acadm | Bckdha | Morn2 | Fam120a | |
| | Mfge8 | B9d1 | Glrx2 | Efnb1 | Zfp664 | Mrfap1 | |

Long-term MEFs

| | | | | | | | |
|---|---|---|---|---|---|---|---|
| Rps3a3 | Cks1b | Utf1 | Crabp1 | Nop16 | Manf | Rplp1 | Cox6a1 |
| Timp1 | Pin1 | Trappc4 | Pfdn1 | Tacc3 | Psmc2 | Srsf3 | Ppm1g |
| Bex1 | Ccng1 | Vdac2 | Atp5b | Ncl | Dnlz | Psma5 | Nosip |
| Rhox5 | Tpi1 | Mrps6 | Hspa9 | Naca | Rps25 | Polr2e | Ola1 |
| Gm15459 | Eif4ebp1 | Gm10039 | Nedd8 | Hint1 | Pdrg1 | Eif3l | Gtf2f2 |
| S100a6 | Tubb6 | Snrpe | Ube2a | Rcn2 | Steap1 | Snrpa | Hprt |
| Gm10320 | Txnl4a | Ruvbl2 | Nsmce1 | Pgd | Snx5 | Rps4x | Sec13 |
| Gsto1 | Cdkn2a | Txnrd1 | Rpl23a | Mrpl11 | Rtn4 | Farsa | Ndufs6 |
| Gm11942 | Npm1 | Actb | Psmd12 | Rps17 | Csnk2b | Rpl17 | Eif3g |
| S100a4 | Cenpa | Snrpa1 | Dynll1 | Ftl1 | Nab2 | Mrps15 | Brix1 |
| Gm10260 | Tagln | Mrto4 | Rps20 | Strap | Hcfc1r1 | Cisd1 | Timm10 |
| Mif | Lgals1 | Abracl | Rhoc | Atp5fl | Eif1a | Eif2s2 | Mips14 |
| Esd | Tmsb4x | Pgk1 | Pdlim1 | Idh3a | Cap1 | Arpc5 | Sf3b4 |
| Gm15772 | Hmgn1 | Ngf | Cct5 | Ctxn1 | Fhl2 | Mrpl42 | Prps1 |
| Anxa1 | Atp5g3 | Cct3 | Phf5a | Avpi1 | Pam16 | Noct | Emc8 |
| Ctgf | Acot7 | Hbegf | Glrx3 | Rps8 | Psmb5 | Txndc9 | Ndufs4 |
| Rps27l | Ranbp1 | Rack1 | Sh3bgrl3 | Stip1 | Chchd1 | Mrpl35 | Uba3 |
| Pkm | Plaur | S100a11 | Pomp | Cdca8 | Dtymk | Nt5c | Srm |
| Bex3 | Vim | Eno1b | Nudcd2 | Mdm2 | Bud31 | Snrpg | Gtf2h5 |
| Txn1 | Cnih4 | Cox5a | Apoc1 | Eif2b3 | Rassf1 | Eif3i | Mrpl17 |
| Tagln2 | Anxa3 | Timm17a | Nmd3 | Arl6ip1 | Rbm8a | Rpl7l1 | Selenof |
| Tnfrsf12a | Tnfrsf11b | Eloc | Rpl19 | Rps3 | Snu13 | Tgif1 | Praf2 |
| Ldha | Dctpp1 | Mtch2 | Cacybp | Capg | Snrpd2 | Rab11a | Med7 |
| Selenoh | Cnn2 | Fkbp3 | Ddx39 | Hspe1 | Mthfd2 | Nip7 | Tuba1a |
| Serpinb2 | Eif5a | 2810025M15Rik | Hnrnpc | Edf1 | Gins2 | Plp2 | Tspan4 |
| Gm28438 | Ass1 | Slc25a3 | Spp1 | Calr | Hsd17b12 | Vps29 | Degs1 |
| Tex19.1 | Krt18 | Rps13 | Cstb | Spc24 | Rplp0 | Dph3 | Rps26 |
| Gm10263 | Cdc20 | Rpl7a | Cox7b | Rps24-ps3 | Bzw1 | Ndufb6 | Ppil3 |
| Tubb5 | Psma6 | Gm11273 | Tes | Prdx2 | Psmd13 | Lap3 | Dnaja2 |
| Birc5 | Ccnb2 | Pa2g4 | Lxn | Shmt2 | Denr | Naa38 | Itgb1bp1 |
| Ran | Prelid1 | Thyn1 | Nasp | 2810004N23Rik | Atpif1 | Zyx | Cldn4 |
| Anxa2 | AA465934 | Cdk4 | Atp5o | Lamtor1 | Cox7a2 | Sae1 | Commd2 |
| Gsta4 | Cct8 | Eif1ax | Rpl39 | 2010107E04Rik | Ptrh2 | Rpl30 | Nol7 |
| Nme1 | Ppia | Serpine1 | Eif4a3 | Yrdc | Mybbp1a | Tpm2 | Cops5 |
| Trap1a | Bola2 | Psma1 | Gars | Commd3 | Nsun2 | Uqcrb | Txndc17 |
| Rrm2 | Eef1b2 | Cct7 | Gjb3 | Pebp1 | Mrpl30 | Ccdc58 | Txn2 |
| Prdx1 | Dut | Btf3 | Mrpl20 | Ccna2 | Aimp1 | Rpl6 | Prdx4 |
| Il11 | Ap1s1 | Hspd1 | Elob | Perp | Emc6 | Gpx1 | Wdr12 |
| Tm4sf1 | Rpsa-ps10 | Gng2 | Ptgr1 | Tmem126a | Arpp19 | Ppp1r11 | Prdx5 |
| Tuba1c | Psma2 | Mtpn | Acta2 | Rps5 | Snx3 | Thoc7 | Vta1 |
| Tuba1b | Cct4 | Tomm40 | Eif3d | Fcf1 | Coq7 | Cdc37 | Alad |
| Eno1 | Hmga2 | Ccnb1 | Bdnf | Atp6v1g1 | Tmco1 | Polr2f | Imp4 |
| Cks2 | Psmd8 | Slc25a5 | Cops6 | Dars | Rars | Nradd | Exosc8 |
| Psat1 | Pclaf | Psmb3 | Pno1 | Lsm5 | Phb2 | Arpc2 | Mrpl39 |
| Ube2c | Snrpd1 | Tyms | Fam162a | Tpm4 | 1810022K09Rik | Mrpl57 | Rpl22 |
| Cldn3 | Bax | Rpl13a | Hnrnpab | Cct6a | Apex1 | Gnl3 | Nras |
| Fabp3 | Rpl27 | Tbca | Mrpl13 | Rpl34-ps1 | Tpm1 | Vbp1 | |
| Hat1 | Inhba | Sgk1 | Rps12 | Mrpl28 | Rsl1d1 | Pmm1 | |
| Mrpl12 | Psph | Aldoa | Rpl11 | Sssca1 | Rrp9 | Rps15a | |
| Eif2s1 | Gm1673 | Mtap | Fkbp1a | Hspb1 | Psmb6 | Mob4 | |
| Cfl1 | Nap1l1 | Actg1 | Eef1d | Rgs16 | Bag2 | Atxn10 | |
| Myl12a | Pttg1 | Rps4l | Rplp2 | Rpl9 | Psmc1 | Usp39 | |
| Tubb4b | Eef1e1 | Gmnn | Nme4 | Paics | Nup35 | Zfp593 | |
| Clic1 | Srp14 | Prdx6 | Aurka | Ciapin1 | Psmb1 | Hikeshi | |
| Cdk1 | Psmd14 | Med21 | Aaas | Mrpl51 | Prss23 | Tars | |
| Aprt | Bri3bp | Dnph1 | Fosl1 | Elof1 | Ndufa8 | Rpl28 | |
| Gm4366 | Asns | Pfdn4 | Ndufb8 | Mrps18a | Ak1 | Erh | |
| Hmga1 | Rps10 | 1110008F13Rik | Lsm8 | Tcp1 | Bcap31 | Rps15 | |
| Vmp1 | C1qbp | Lsm2 | Timm50 | Tk1 | Sigmar1 | Phgdh | |
| Crlf1 | Cnih1 | Pfn1 | Hn1 | Phlda3 | Ak6 | Krt8 | |
| Gapdh | Rpl12 | Slc16a3 | 2200002D01Rik | Zwint | 1500009L16Rik | Cox17 | |
| Banf1 | Nhp2 | Psmc6 | Serbp1 | Rheb | Tipin | Fez2 | |
| Rpl18 | Cct2 | Capzb | Ankrd1 | Chmp6 | Slirp | Tbpl1 | |
| Galk1 | Cdkn2b | Txnl1 | Rbx1 | Ndufa7 | Snx7 | Arhgdia | |
| | Rpl22l1 | Uqcrq | Itga5 | Cox6b1 | Pmf1 | Dda1 | |

Embryonic mesenchyme

| | | | | | | | |
|---|---|---|---|---|---|---|---|
| Matn4 | S100b | Hmgn1 | Pdap1 | Prelid1 | Bub3 | Peg3 | Rpl31 |
| Matn1 | Crabp1 | 1110004F10Rik | SdhaK | 2210013O21Rik | Psmb6 | Atp5g1 | Rps11 |
| Col9a1 | Fibin | Gm1673 | Hpf1 | Serf1 | Thoc3 | Slc25a4 | mt-Nd1 |
| Col9a3 | Siva1 | Psmd6 | Rer1 | Pdxdc1 | 2310036O22Rik | Nop58 | Rpl10 |
| Cnmd | Gpc3 | Ssr2 | Tmed1 | Srsf3 | Rpl36al | Chchd2 | Rps5 |
| Asb4 | Cthrc1 | Sub1 | Mif | Gnl3 | Limd2 | Arf1 | Rpl26 |
| Col9a2 | Tpi1 | H19 | Hnrnpm | Ndufa4 | Hnrnpa2b1 | Ier3ip1 | Rps8 |
| Wwp2 | Hnrnpd | Grb10 | Gars | Meg3 | Snx17 | Rps27a | Rps15a |
| Sox9 | Col11a1 | Prpf19 | Capn6 | Fkbp4 | Elp2 | Calr | Rplp0 |
| Col2a1 | Cpc | Elovl6 | Fus | Rcn1 | Atp5a1 | Swi5 | Rpl13 |
| Nnat | Fgfr3 | Dek | Psma7 | Itm2a | Slirp | Rps9 | Rps25 |
| Hapln1 | Eno1 | Pkm | Gstm5 | Hsp90b1 | Atp5k | Cox5a | Rpl18a |
| Cytl1 | Ccnd1 | Snrpd3 | Fkbp11 | Ugdh | Blmh | Rpl18 | Rps14 |
| Cd24a | Rflna | Ptov1 | Skp1a | Ddx39b | Nasp | Ndrg2 | Dlk1 |
| Mest | Rangap1 | Psmc4 | Apex1 | Hspe1 | Hint1 | Usmg5 | Rpl41 |
| Mia | Maged2 | Nop10 | Papss1 | Sec61b | Ddx39 | Rps2 | |
| Bex2 | Mlf2 | Tial1 | Cct3 | Ptma | Ap1m1 | Tmem258 | |
| Mpz | Snrpa1 | Lman1 | Mrpl15 | Atxn10 | Eif5a | Serbp1 | |
| Cdkn1c | H2afx | Tceal9 | Nsfl1c | Ranbp1 | Galk1 | Rps13 | |
| Papss2 | Cacybp | Hspd1 | Anapc11 | Cct6a | Polr2i | Elob | |
| Stmn1 | Gale | Eef1g | Mcm7 | Mrpl34 | Tspan4 | Dad1 | |
| Ldha | Pdrg1 | Krtcap2 | Npm1 | Serpinh1 | Atp5f1 | Rpsa | |
| Plod2 | P4hb | Snap47 | Snhg6 | Dcakd | Rpl11 | Gapdh | |
| Cdk4 | Ldhb | Cks1b | Rnf7 | Atp5j | Rpl14 | Gnas | |
| Slc26a2 | Srm | Tmem97 | Ssrp1 | Tecr | Luc7l3 | Tsc22d1 | |
| Bex3 | Susd5 | Kdelr2 | Cnpy2 | Serp1 | Ube2e3 | Igf2 | |
| Epyc | Ltv1 | Selenoh | Tfg | Nme1 | Ywhab | Id3 | |
| Pdia6 | Tubb5 | Vdac3 | Lrc59 | Hnrnpc | Akr1a1 | Cfl1 | |
| Ss18l2 | Gadd45gip1 | Srsf2 | Mdk | Atp5o | Rps26 | Hsp90ab1 | |
| | Ccnd2 | Srp72 | Klhl13 | Snrpa | Ndufc1 | Rps17 | |

Cxcl12 co-expressed

| | | | | | | | |
|---|---|---|---|---|---|---|---|
| Il1r1 | Il13ra1 | H6pd | C1ra | Gas6 | Itga11 | Serpina3g | Pkdcc |
| Col3a1 | Apln | Isg15 | C1s1 | Sfrp1 | Col12a1 | Serpina3n | Epas1 |
| Col5a2 | Hs6st2 | Steap4 | P3h3 | Slc7a2 | Selm | Ghr | Colec12 |
| Igfbp5 | Bgn | Emilin1 | Fxyd1 | Comp | Ebf1 | Osmr | Egr1 |
| Sned1 | Slc16a2 | Htra3 | Rcn3 | Bst2 | Slfn2 | Lifr | Lox |
| Ifi203 | Capn6 | Nsg1 | Fcgrt | Rnf150 | Col1a1 | Snhg18 | Iigp1 |
| Nenf | Gpm6b | Sod3 | Saa3 | Ier2 | Igfbp4 | Ly6e | Synpo |
| Pfkfb3 | Cp | Pdgfra | Prss23 | Nfix | Mrc2 | A4galt | Pdgfrb |
| 1110008P14Rik | Dclk1 | Cxcl5 | P2ry6 | Junb | Timp2 | Fbln1 | Efemp2 |
| Lcn2 | Mme | Cxcl1 | Adm | Mmp2 | Lgals3bp | Pdzrn4 | Pcsk5 |
| Serping1 | Ptx3 | Plac8 | Il4ra | Mt2 | Sfrp1 | Rtp4 | Ifit3 |
| Ube2l6 | Tbx15 | Spp1 | Ifitm2 | Mt1 | Aspn | Mylk | Ifit1 |
| Fibin | Slc16a1 | Pkd2 | H19 | Cdh11 | Ogn | Fstl1 | |
| B2m | Vcam1 | Tgfbr3 | Igf2 | Hp | S1pr3 | Nfkbiz | |
| Eid1 | Penk | Oasl2 | Rspo3 | Stc1 | Cxcl14 | Abi3bp | |
| Fgf7 | Svep1 | Col1a2 | Bicc1 | Pdlim2 | Gas1 | Tmem45a | |
| Cpxm1 | Ugcg | Ptn | Col6a1 | Slc39a14 | Vcan | Col8a1 | |
| Ism1 | Plpp3 | Rarres2 | Aes | Tsc22d1 | Pik3r1 | Adamts5 | |
| Cst3 | Podn | Tmem176a | Igf1 | Mmp13 | Il6st | Kcnj15 | |
| Lbp | Hivep3 | Loxl3 | Dram1 | Mmp3 | Stxbp6 | Fndc1 | |
| Wisp2 | Col8a2 | Cyp26b1 | Dcn | Clmp | Hif1a | Sod2 | |
| Zbp1 | Nbl1 | Antxr1 | Lum | Nnmt | Zfp3611 | Thbs2 | |
| Srpx | Mfap2 | Slc6a6 | Ndufa4l2 | Islr | Npc2 | Angptl4 | |
| | Dhrs3 | Cxcl12 | Lrp1 | Loxl1 | Ltbp2 | Cyp1b1 | |

Ifitm1 co-expressed

| | | | | | | | |
|---|---|---|---|---|---|---|---|
| 1500015O10Rik | Serping1 | Cp | Ifitm2 | 1500009L16Rik | Ctsh | Tgfbi | Ap0d |
| Crocc2 | Cst3 | Gper1 | Ifitm1 | Scara5 | Zic1 | Hif1a | Abi3bp |
| Sned1 | Ptgis | Gng11 | H19 | Zic5 | Zic4 | Aspg | Epha3 |
| Fmod | Slc16a2 | Cemip | Akap12 | Mmp13 | Ebf1 | Fbln1 | Smoc2 |
| | Fabp5 | Adm | Gja1 | Clmp | Sfrp4 | Kng2 | Thbs2 |
| | | | | | | | Epas1 |
| | | | | | | | Prdm6 |

Matn4 co-expressed

| | | | | | | | |
|---|---|---|---|---|---|---|---|
| Spats2l | Kcns1 | Penk | Eln | Pdgfrl | Mfap4 | Igfbp4 | Nov |
| Igfbp5 | Matn4 | Mfap2 | Cpxm2 | Igfbp3 | | | |

2-cell

| | | | | | | | |
|---|---|---|---|---|---|---|---|
| Tel1b1 | Pxt1 | Omt2b | Inpp4a | Stbd1 | Ampd3 | Stk36 | Rnf182 |
| Dusp7 | Smad3 | Obox5 | NA.15103 | NA.13579 | NA.15121 | Sytl4 | NA.12407 |
| Zbed3 | B4galt6 | Itga9 | Mllt3 | Man1c1 | Angel2 | Tmem92 | Ptpre |
| Tcl1b2 | X7420426K07Rik | Ptprr | Mcc | Sh3bp1 | Sipa1l1 | Akt3 | Zcchc2 |
| Gm839 | Creld1 | NA.15153 | Slc15a5 | Kit | Gm21762 | X9130023H24Rik | Tcstv1 |
| NA.13991 | Lbx1 | Hmces | Fam167a | Nos1ap | NA.9588 | Hoxa7 | Spesp1 |
| Gm1965 | Gad2 | Mfsd2a | Pip5k1b | Mvb12b | Gm13023 | Coro2b | Ppp1r3d |
| Phf1 | Mn1 | Tgfb2 | Bmp5 | Prr5l | Olfr288 | NA.15065 | Grip1 |
| Tcl1b3 | Ccdc69 | Plekhg1 | NA.15072 | Adm2 | Gm12735 | Ctdspl | Hsd17b13 |
| Siah2 | Pak7 | Mcu | O0sp1 | Igsf11 | H2.Q6 | AU015836 | Tet3 |
| Tcl1b4 | Stradb | Myo3a | Vil1 | Aida | NA.15138 | Cngb1 | Wdr25 |
| Phc2 | Rfpl4 | Gm11131 | NA.2207 | Rimkla | Wasf3 | NA.10579 | Mapkbp1 |
| Tel1 | Fam43b | Zscan4d | Bcorl1 | Jazf1 | Polm | Usp46 | Fchsd2 |
| Tbx19 | Gli3 | Bmp2k | Zfp513 | Tshz1 | Man2a2 | Cdc42se2 | Fam19a2 |
| Obox3 | Grm2 | Btg4 | Plxnc1 | Gng3 | Gm9125 | Gyg | Ssh1 |
| NA.6855 | Parp12 | Fyn | F2r | Dpysl3 | Usp21 | Igdcc3 | Errfi1 |
| Gm12789 | D6Ertd474e | NA.13288 | Kcnk18 | Gfod1 | Tmc8 | Plag1 | Fbxw22 |
| Wee2 | Reep2 | Pik3cd | Klhl8 | Tesc | Ccdc92 | Arntl2 | Ajap1 |
| Bcl2l10 | Btbd2 | Adcy5 | Cby3 | Oosp2 | Lrrc4 | Fbxw14 | Gm20767 |
| Rph3a | Gpr68 | Smpd3 | Cpa1 | Syt11 | NA.10324 | Catsperg1 | Epha3 |
| Gm6507 | Slc45a3 | Pld1 | Sbk1 | Tmcc3 | Sipa1l2 | Itpk1 | Dpp10 |
| Th | Iqca | NA.80 | Zscan4c | Elavl2 | Nlrp4e | Prss46 | Slc30a3 |
| Musk | Tubg2 | AU016765 | Slc1a4 | Plek | Gja3 | Spire1 | Gm28078 |
| NA.10366 | Kcnh1 | Oas1d | Ablim2 | Spocd1 | Ramp3 | Nlgn1 | Itga8 |
| Tmcc2 | X2210019I11Rik | Gm17751 | Mansc1 | Dennd3 | Orai1 | Dbndd1 | NA.15123 |
| Fa2h | Accsl | Krt84 | NA.15114 | Lrp1b | Sufu | A630095E13Rik | Taf9b |
| Spry4 | X2010107G23Rik | Unc13c | Peak1 | Pcdh15 | Lef1 | Nr2e1 | Plxna4 |
| Tbxa2r | B4galt2 | Fmn2 | Colgalt2 | Nav2 | NA.1519 | Gm13103 | Mfsd6 |
| Rims1 | AC126035.1 | Angptl2 | Zfp30 | NA.10749 | Nav3 | Lhx8 | Pou4f1 |
| NA.4062 | Usp17lc | X9530082P21Rik | Rapgef5 | D6Ertd527e | Gstm5 | Nrep | Fgfrl1 |
| Papd7 | Rab3d | Pdgfrl | Ctif | Timd4 | Smox | Pla2g4c | Evl |
| NA.14200 | NA.10463 | Rasd2 | Eif4e1b | Efha5 | X4933404O12Rik | Rasa4 | Gdf9 |
| NA.7294 | Eif4e3 | Per3 | Ifitm6 | Rspo2 | Vps9d1 | AI987944 | Dnasel13 |
| Gm11827 | Prkaca | Smim14 | Cob1 | Maml1 | Sort1 | NA.12447 | Shroom4 |
| NA.5539 | NA.12521 | Hipk2 | Zfp46 | Lsm10 | Shank2 | Prmt2 | Fbxo43 |
| NA.3541 | Mmp2 | Slc24a3 | Ppp1r9b | Slc6a7 | X4933415A04Rik | Dact3 | Unc13b |
| Usp17lb | Axin2 | AA415398 | Mypop | Gm15668 | Fam117a | Magi1 | Scg3 |
| Bmp15 | Fzd2 | St6gal1 | Mllt11 | Lrrc8a | Jade2 | Gm13191 | Fgf7 |
| Tfap2e | Cbx2 | Ctdsp1 | Cdh4 | Txndc2 | Ptcra | Emilin2 | C87499 |
| Rbm38 | Fmnl3 | Adarb2 | Ccnj1 | Gm28784 | Dpf1 | Smagp | Tubb3 |
| Zdhhc8 | Hpcal1 | Foxm1 | Midn | Efcab12 | Pld6 | Spin1 | NA.232 |
| Lzts1 | Prrg1 | Adamtsl1 | Tspan5 | Tef | Ets2 | Tbc1d8 | Limd1 |
| Tcl1b5 | Sebox | Arhgap20os | Gbas | Nhsl1 | Elmod3 | Gphn | Esyt1 |
| Slc03a1 | Obox1 | Lingo2 | Ttbk1 | Glis3 | Acot3 | Synm | AF067061 |
| Dclk2 | Zfp957 | Tox3 | B4galnt4 | Mark2 | Apol7b | Tmem72 | Trak1 |
| Tulp3 | Taar2 | Bmp6 | Gm11381 | Apela | Pacs2 | Fkbp5 | Slc22a23 |
| NA.1891 | Rassf5 | Fsd1 | Rragc | Adam33 | Tmem108 | Clvs2 | |
| NA.15124 | Afap1l2 | Gm21818 | Nrp1 | Cacna1h | Dmwd | Rnf220 | |
| Rgs17 | Tmem184b | Tcf20 | AU022751 | AI854703 | Ubash3b | Platr22 | |
| Zfp352 | Omt2a | E330012B07Rik | Nceh1 | Zfp703 | X2310061I04Rik | B4galt4 | |
| NA.10433 | Trim75 | Tob2 | Lrrc16a | Creb3l4 | Fbxw24 | Sgms2 | |
| Cmya5 | Pcdh9 | X4933427D06Rik | Oosp3 | Fzd7 | Ccno | Aicda | |
| Cdr2 | Foxj2 | Dnah7c | Fam199x | Mmp19 | ACox3 | Glis1 | |
| Mfap2 | Tmtc1 | Angel1 | Myadml2 | Khdc1b | BC147527 | E330021D16Rik | |
| Gna12 | Prkd1 | Prlr | Ms4a1 | Prrx2 | NA.3893 | Oog1 | |
| Cntnap1 | Ppm1h | Ccdc6 | Diras2 | Kmt2d | Eef2k | Sh3rf3 | |
| NA.10280 | NA.9512 | Shb | Pde4c | Prss45 | Farp1 | Ttyh3 | |
| Mesp2 | Nrsn2 | NA.7047 | Pptc7 | Trim7 | E330034G19Rik | C330021F23Rik | |
| Vrtn | Trim60 | Ybx2 | D13Ertd608e | Il7 | Fbxw18 | N4bp1 | |
| Parp10 | Slc25a48 | Kif17 | Gm16050 | Sbf2 | Kpna7 | Dcakd | |
| Fam222a | Snph | Lmx1a | Fam131a | Tcf7 | NA.6131 | Obox2 | |
| Pkd2l2 | Antxr1 | Pou2f2 | Obox7 | Ksr1 | Tbc1d2b | Gramd2 | |
| Samd10 | B020004C17Rik | Ninj1 | Cyth1 | Rundc3b | Fhod3 | Tmem180 | |
| Tbx4 | Derl3 | Cables1 | Rnf26 | NA.1579 | Pygo1 | Prr32 | |
| | Ahdc1 | Meis2 | Nobox | Lmol | Ap3m2 | Ccdc88a | |

4-cell

| | | | | | | | |
|---|---|---|---|---|---|---|---|
| X1700019E08Rik | Esam | Otop1 | NA.15084 | Tmem210 | E030044B06Rik | Ptdss2 | NA.9870 |
| Gcm1 | Tmc5 | Caap1 | Eif4e | Pdlim4 | Arrdc3 | Vmn1r90 | Toporsl |
| Gm26815 | Kcne3 | Tc2n | Ttc30a1 | Lamp2 | Spink2 | Cracr2b | Mlf1 |
| Hand1 | Dnmt3bos | Kcnf1 | Ccr4 | X1810034E14Rik | Rhoq | P3h4 | GM26745 |
| Esx1 | Nags | Slc38a2 | Hoxb9 | Pcolce2 | Ddx60 | Gm26632 | X1700092M07Rik |
| NA.13936 | Zfp644 | Gm9918 | Tmem5 | NA.551 | Cdkn2a | Clec2g | Akap12 |
| Mbnl3 | Tspan6 | Spata25 | Zfp273 | Pgm2l1 | Psma8 | Gm16302 | Cnnm1 |
| Tgfb1 | Gm9732 | Myc | Nabp1 | Chic1 | Bcst2 | Elf4 | Tmem63a |
| NA.11398 | Sycp1 | C2cd4b | Adam19 | Trim40 | Gm15128 | Slc25a46 | Olfr815 |
| Ltb | NA.9651 | Gm595 | Ythdc2 | Rmdn2 | Dppa2 | Tmem47 | Tacr2 |
| X1700003E16Rik | AI606181 | Rbm41 | Gramd1a | Ddit4 | Mcttl20 | Sowahc | Adamtsl4 |
| Pi16 | Foxa1 | NA.12611 | Rnf11 | Tram1l1 | Ei24 | Mxra7 | Rdh10 |
| Calm5 | Ccdc89 | Cacng7 | AC133103.1 | Ptprcap | Nr2c2 | Ap1s3 | Pxdc1 |
| Tmem37 | Nrg2 | Jakmip1 | Ctsl | Epm2a | D930016D06Rik | Hfm1 | Cyr61 |
| Olfr836 | Eid1 | NA.5175 | Crabp1 | H3f3b | X4930503E14Rik | Ccdc57 | Prpf4b |
| Map7d1 | Rtn4r | Zswim5 | Uhrf2 | Agbl2 | Sox15 | Wipf1 | X1700123I01Rik |
| Tceal8 | P4ha3 | Obox8 | NA.556 | Igfbp3 | Six4 | NA.11442 | NA.1350 |
| Nfatc1 | Cav1 | Syne3 | Fam122b | Upk3b | Ramp2 | Wdr5b | NA.9846 |
| Wbp5 | NA.7320 | Lrrc15 | Cbfb | X6030443J06Rik | NA.44 | Plin5 | Unc5cl |
| NA.7187 | Tex15 | Irak1bp1 | Lpar6 | Robo4 | Gm5773 | Dixdc1 | Zfp948 |
| Tcf23 | Rbm12 | Kcnk5 | Gm6871 | Ddias | Slc12a2 | Gm1123 | NA.13261 |
| Noto | Bex1 | Pdlim3 | Gm16010 | Gm15389 | Slc35f5 | Brwd3 | Tdpoz4 |
| Pet2 | NA.8609 | Mat2a | Ahi1 | Lamc2 | Lbhd1 | Amigo2 | Zfp799 |
| Nupr1 | Gm11961 | Gm14443 | Spaca6 | Calb2 | H2afx | NA.5634 | Naf1 |
| 43353 | Fgr | Klf17 | Ube2e3 | NA.337 | Arl4c | AC125149.1 | NA.9901 |
| Myh7 | X3110021N24Rik | Lix1l | Xcr1 | Mtmr6 | NA.10058 | Ppwd1 | NA.7995 |
| Zfp457 | X9030407P20Rik | Trpd52l3 | Zfp874a | Fam65c | Fkbp10 | Gm26522 | Gm10509 |
| Nxf2 | Tbc1d12 | Gm14124 | Cenpq | Lrif1 | Krt28 | Rasgef1a | Gm28875 |
| Prdm14 | NA.15089 | Fscn1 | NA.3213 | Ehd2 | Set | Zfp874b | Rnd2 |
| Dlx3 | NA.7248 | Platr25 | Ggt7 | Chrnb1 | Cbx3 | Cyb561d1 | Nudt16 |
| X4930502E18Rik | Abcb5 | Trim2 | Zfp85 | Cpz | Sdc3 | Ttc29 | Rsrp1 |
| X1700065O20Rik | Sphk1 | Tuba3b | Ctsk | Prcp | Cyp2j6 | Gm7334 | Uty |
| Wnt10b | Hivep2 | Wnk3 | Gm28043 | Slc24a4 | Endog | NA.15101 | Vgf |
| Bbs12 | Bean1 | Map7d2 | Ctag2 | Zfp950 | X9430020K01Rik | Uaca | NA.12375 |
| Lrrc19 | Spsb4 | Morc4 | Olfr143 | Mesdc1 | Atp2c2 | NA.8430 | NA.2730 |
| Phyhip1 | NA.9430 | Kalrnm | Mier3 | Zfp729a | Gm10550 | Obox6 | Unc45b |
| Pla2g4a | Armcx4 | NA.9316 | Isl1 | Gm8104 | Col17a1 | Nanos2 | Pigw |
| Tceal7 | Zfp758 | Platr3 | Pank3 | NA.539 | Wsb1 | X4930505A04Rik | D730003I15Rik |
| Siah1a | Tnfrsf11a | Cyp1a1 | Ap4b1 | NA.15064 | Slc19a1 | Trpc5os | Gm4285 |
| Trim56 | NA.5916 | Sox30 | Pik3c2a | Hmha1 | Rsph9 | Rnpc3 | Slfn9 |
| Magea8 | NA.15077 | X3222401L13Rik | Capn9 | Wdr54 | Zfand5 | A930003A15Rik | Edaradd |
| Hes1 | Pkdl13 | Gm16185 | Foxf1 | Jrkl | Sepp1 | Pnn | Slc5a3 |
| Btg1 | Hic1 | NA.264 | Tnfsf13b | Pax6 | Relb | NA.4962 | L3mbtl3 |
| Zfp239 | Chrnd | Gm17056 | NA.1494 | Etnk1 | Gm2399 | Hnrnpll | Pln |
| Gm10226 | NA.407 | Hsd17b14 | Rnft1 | Cebpa | Atg3 | NA.186 | Gm11508 |
| P2ry4 | Magea5 | Tmem229b | Notch4 | Hsf3 | Prss36 | Ctsb | NA.4305 |
| Usp9y | X1700019B21Rik | Usp44 | Gm12315 | Fzd4 | NA.222 | NA.10139 | |
| Gm5930 | Pm20d2 | Cryba1 | Aebp1 | Hkdc1 | Elovl3 | X4930447C04Rik | |
| Sox21 | Sec16b | Gbx1 | Tex37 | Cldn10 | Npas2 | NA.10456 | |
| Selenbp1 | Mast1 | Gm8126 | Rhox9 | Smim10l1 | Nme5 | Gabra4 | |
| Gm6526 | NA.1742 | Nufip2 | X4930432K21Rik | Gm26782 | Mysm1 | Col5a3 | |
| NA.15085 | Nrxn2 | Uba1y | Soat2 | Zfp945 | C130026I21Rik | Pbld2 | |
| X1700049G17Rik | Acsl4 | Irf2bpl | Hesx1 | Slc26a10 | NA.6224 | Cd81 | |
| Gm53 | B230219D22Rik | Aim2 | Vat1 | Gm6268 | Lrrc58 | Lrrc46 | |
| Mycn | Gm15518 | NA.4044 | Nlrp6 | NA.180 | NA.7446 | Gm7073 | |
| Gm15097 | Ptprz1 | Ranbp6 | Hrk | Card14 | Bhlhb9 | Fam228b | |
| NA.10436 | NA.15112 | Id4 | Prrt1 | Rimklb | Mplkip | Ctsc | |
| Fbn1 | A930017K11Rik | Platr23 | Zfp40 | Zfp953 | Sparcl1 | Mrap | |
| Adgrb1 | NA.4501 | Spic | Arg1 | Fgf4 | NA.7433 | Grik1 | |
| Klf2 | Mbnl1 | Gm17404 | Man2c1os | Tenm3 | Cfap73 | Rb1cc1 | |
| Fam212a | B3gnt8 | Chadl | Gm5532 | Mir17hg | Gm14168 | NA.7081 | |
| Fgf3 | Gm29087 | Ccdc152 | Hnrnpa1 | Ambn | Slc16a14 | Dgat2 | |
| Tcp11l2 | Dsc3 | Olfm3 | Tnfrsf1a | Btbd3 | Avl9 | AC133103.5 | |
| Sema6b | Irf7 | NA.12133 | Ell2 | Fbln2 | Ogn | Lcat | |
| | Plek2 | Ffar4 | Ikzf5 | Per2 | X1700019G24Rik | NA.4426 | |

8-cell

| | | | | | | | |
|---|---|---|---|---|---|---|---|
| NA.7110 | Xist | Lif | BC052040 | Zfp936 | Slc7a7 | NA.13976 | NA.3445 |
| Cyp2d9 | Arhgef16 | Qpct | Ly6a | NA.5874 | Gm14582 | Arfip2 | Plekhf1 |
| Ackr3 | NA.689 | NA.88 | Prdx6 | Vpreb3 | Adgrg3 | NA.9630 | Cd59a |
| Perp | Kcnv2 | Nr4a1 | Chmp4c | Vsx1 | NA.6826 | Pmaip1 | Tfcp2l1 |
| Cst13 | Fkbp9 | Grin1 | X2410141K09Rik | Kctd1 | Rpl39 | Gcfc2 | Gm13212 |
| NA.9215 | Gas6 | Nup62cl | Fbxl20 | Ccdc84 | Nog | Gm13051 | Parp16 |
| Cpne3 | H60b | Trmt10b | Tyms | Gsta1 | Gm26584 | Gm19667 | Nln |
| Dok2 | Gm26692 | Exoc3l4 | Eps8l2 | Zfp275 | Fbp2 | NA.10925 | NA.1527 |
| Cd28 | Slc12a7 | I830077J02Rik | A230083G16Rik | Hopx | Clcnka | NA.5489 | NA.4804 |
| Phla3 | Plagl1 | NA.7942 | Prkra | NA.3556 | Gm14401 | Lrpap1 | NA.3235 |
| Cartpt | Ppm1k | Hsh2d | Gm9776 | NA.3384 | Mef2d | Reg1 | Esrp2 |
| Cthrc1 | Ppfibp2 | Cd300a | Lasp1 | Vgll4 | Myo15b | Golga7 | Ly96 |
| Msc | Gm12705 | Ptpn6 | Cstf3 | Ptdss1 | Cdc42ep3 | Chordc1 | X9030624J02Rik |
| Stxbp6 | Vav1 | Gm6020 | Akr1c21 | NA.6297 | NA.2700 | Il22ra2 | NA.3453 |
| NA.810 | NA.8401 | Siglecg | Hoxa9 | Plcd1 | Hhex | Gm11630 | Mfsd8 |
| Stfa2l1 | Pla2g7 | Prrg3 | Ecel1 | Gm26514 | Gm12289 | Ehd1 | Slc45a4 |
| Pdzd3 | Dkk1 | Zfp932 | NA.4219 | NA.4998 | Hmga2 | Pkp2 | Urgcp |
| Gm27204 | Sbp | Gm21060 | X9430060I03Rik | NA.7408 | Zfp429 | Pdcd6 | Igbp1 |
| Anxa3 | Hsd1Tb1 | X1010001N08Rik | Mocos | Gm16503 | Pou5f1 | Efna1 | Lgals8 |
| NA.1015 | Rragd | Rnf138 | Slc6a14 | NA.10479 | 43351 | Ttc39b | NA.4193 |
| Vrk2 | Tmem81 | Sync | Smpdl3a | Plxnb2 | Adgrf3 | Cyba | Atp6v0e2 |
| Npy | H60c | Xkr9 | Nudt11 | Slc10a4 | Fam198b | NA.14015 | Chpt1 |
| Tspan1 | Svil | Gm17655 | Krt7 | Sall1 | Hprt | Cd209e | NA.588 |
| Stard4 | Pramel5 | Eno2 | NA.5168 | NA.12148 | NA.711 | NA.9466 | Adam4 |
| Lect1 | Irf5 | Amph | Ormdl1 | C3ar1 | Grk6 | Gm20515 | Zfp607 |
| Gyltl1b | Dcaf12l1 | Ccdc150 | NA.4188 | Gm13062 | Atp2b1 | A530040E14Rik | Atp6v0a4 |
| Nxpe5 | Gm4131 | Cdc42ep1 | Hspa8 | Fndc3c1 | Sat1 | NA.4431 | Arhgap27 |
| Dynap | X4930550L24Rik | NA.4813 | Rassf7 | Dpy19l2 | Fam217b | Rnf32 | Cdh1 |
| Gm15446 | Zfp52 | Eda2r | Star | Ano2 | Etohi1 | Ly6g6e | Il17re |
| Zfp934 | NA.3646 | Hes2 | Pkd111 | NA.13900 | G430049J08Rik | Ldb1 | NA.3823 |
| Platr10 | X4930522L14Rik | Etl4 | D930020B18Rik | Iqgap3 | Fam83b | Gm11541 | NA.4035 |
| Amot | Slco2a1 | Vangl1 | Arhgap18 | Sh3d21 | Pde7a | Gm2366 | NA.4009 |
| Id3 | Gm26836 | Atp8b4 | Ppp2r2c | 43160 | NA.4566 | Prr19 | Lpin1 |
| Amotl2 | Ap3b2 | Cav2 | Dennd1b | Akp3 | Cldn4 | Cmtm5 | Atg4c |
| Gm26740 | NA.4112 | Slc29a3 | BC051665 | Glt28d2 | Foxf2 | Tmem45a | Alg13 |
| Abcb1a | NA.10665 | Nradd | Dnal1 | Grn | Pank4 | NA.9621 | Rad23a |
| Diaph2 | Tmem245 | Tmem253 | Klf8 | NA.2621 | B930036N10Rik | NA.336 | Gm26538 |
| Akr1c14 | Pik3r6 | NA.1630 | Gm13235 | Cwh43 | NA.7030 | Gm10687 | Prr15l |
| Cryab | Tsix | Ddah1 | B4galt1 | NA.7337 | Gm26668 | Zfp418 | NA.7290 |
| Il33 | Hsd17b11 | Ano9 | NA.5135 | Sh3tc1 | Gabrd | Gm1976 | Upf3b |
| Slc19a2 | Zfp354a | Acp5 | NA.1892 | Pin1rt1 | Tbx3 | NA.1763 | Slco4c1 |
| Epas1 | Gm1110 | B230312C02Rik | Cks1brt | C030039L03Rik | X9430002A10Rik | NA.7085 | NA.5912 |
| NA.1618 | Bves | Lrrc23 | Lrrc37a | Cald1 | Ctsf | Acyp2 | Emilin1 |
| Pcdhb16 | Xlr | Cux2 | Krt27 | Akap2 | NA.6 | Oxct1 | NA.5335 |
| Bex4 | AI467606 | NA.9543 | Wnt3a | Il13ra1 | Gm27206 | Pigz | Tmem144 |
| Tmem64 | Mtm1 | Gm6712 | Smoc1 | NA.9845 | Rnf208 | Tpd52 | Zfp599 |
| Bmp8b | Ccng1 | NA.7720 | Igsf1 | Sbp1 | Bhmt2 | NA.47 | |
| Gm10139 | Arhgdib | Fam129a | NA.5696 | NA.1027 | NA.2931 | Mllt6 | |
| Gpc4 | Fam124a | NA.2889 | Kcnh | NA.3116 | NA.691 | Plcg1 | |
| Vnn1 | Slc52a3 | Gm10324 | Gm13242 | Alcam | Adam21 | Pnpla2 | |
| Rbms1 | Gm13154 | Slc29a4 | Sema5b | NA.13906 | Serinc1 | Gm15137 | |
| Apob | Suox | NA.2540 | NA.9923 | Inmt | NA.12649 | Dnajc6 | |
| X9330185C12Rik | NA.2957 | Gm12514 | NA.513 | Card11 | Mybpc2 | X2410018L13Rik | |
| Camk4 | Fgf13 | Cd53 | Grhl3 | Asap2 | Runx1 | Actn1 | |
| NA.559 | Parva | Msmo1 | Lpar1 | Smim22 | Vtn | NA.223 | |
| Mpped2 | Casc4 | Ramp1 | NA.3947 | Sycn | Fancb | Rbks | |
| Pof1b | X9230009I02Rik | Postn | Isl2 | Ak7 | Klf10 | Nrtn | |
| Papss2 | F12 | Havcr1 | Fes | Nprl2 | Gm26624 | Fut9 | |
| Tb.x20 | X2210404O09Rik | Ttpa | Nap1l2 | Zfp422 | NA.10303 | Ednrb | |
| Gng2 | S100a11 | Gjb3 | Sh3glb2 | Alg6 | NA.7385 | Zfp458 | |
| Nr2f2 | X5430403G16Rik | Ahsg | Nck2 | Npnt | NA.487 | Itpkb | |
| Rarb | Steap3 | Strada | Gata6 | NA.424 | NA.2929 | NA.11397 | |
| Gm10772 | Matn3 | Reep1 | Slc36a3os | Psrc1 | Rdh5 | NA.1522 | |
| Zfp157 | Slc22a13 | Ncf2 | NA.14579 | Sfrp1 | NA.5637 | NA.9911 | |
| | Fgd4 | NA.4991 | Bok | Ace2 | Vps33b | NA.2756 | |

16-cell

| | | | | | | | |
|---|---|---|---|---|---|---|---|
| Gm2245 | H2afy | Khdc3 | Tbca | Erlec1 | Adam9 | NA.12986 | Nipa1 |
| Fabp5 | Rhob | X4930558J18Rik | Mycl | Slc7a15 | Pomt1 | Egfl7 | Tpp1 |
| Gm17067 | Trip6 | Gm14409 | Phlpp1 | Vcpkmt | Gjb3 | Ormdl1 | Gm4673 |
| Apoa1 | Tmsb4x | Top2b | Sqstm1 | Trim47 | Acad12 | B3gnt3 | Slc35a1 |
| Stat6 | Slc6a13 | Ank2 | Hbegf | Bcl9l | Tmem135 | BC052040 | NA.5230 |
| Capn6 | Plk5 | Nudt10 | Serpinb6a | Evpl | X2610528J11Rik | Paqr5 | Hdac3 |
| Abca1 | Col4a1 | Pvrl1 | Acp1 | Actg1 | BC029214 | Pfn2 | Whamm |
| Gm14305 | Shkbp1 | Anxa9 | Nanog | AU021092 | Them5 | Gm14403 | Gpx2 |
| Eomes | Mgst2 | Hal | Rem1 | Cdk18 | Atp8a1 | Vmn2r29 | Trappc1 |
| Zfp36l1 | Cdc123 | Slc2a1 | Spp1 | Dok2 | Psmg2 | Gstp1 | Tmem198 |
| Sox2 | Dsg2 | Acaa2 | Tex19.2 | Cldn23 | Sik2 | Gm17087 | NA.4039 |
| Sh3bp5 | Mpzl2 | Lyrm9 | Pdzk1ip1 | Nsmaf | Wnt6 | Slc5a2 | X3110052M02Rik |
| Ptgdr | Glrx | S1pr1 | X1700095A21Rik | Cpxm1 | Bre | NA.7316 | Adprh |
| As3mt | Frrs11 | Pgap1 | Camk1 | Impad1 | Elf3 | Npc1 | Thrsp |
| Pmaip1 | Gss | E130012A19Rik | GM14327 | Crip2 | Pigz | Pms1 | NA.10775 |
| Dok1 | Hebp1 | Xbp1 | Bcnd7 | Lamc1 | Itga7 | Sccpdh | Gm26578 |
| Slc37a2 | Sox7 | Zcchc16 | Alg8 | NA.6114 | Lrmp | Spcs3 | NA.3851 |
| Tinagl1 | Cbx4 | Mapt | Nap1l3 | Eps8l1 | Vapb | NA.499 | Aasdhppt |
| Aldh1b1 | Fbxo3 | Arl6ip5 | Vps13c | Camk2d | Bhlha15 | Slc4a2 | Pkp2 |
| Mafb | Pnma2 | Pou2f1 | Epcam | Alcam | Gm10605 | Gatad1 | Plgrkt |
| Lypd8 | Fam92a | Cited4 | Dpysl4 | Ass1 | Hsp90aa1 | Atp2a3 | NA.14210 |
| BC048679 | Ddx3y | Tbx1 | Fas | Mospd2 | Nsdhl | Fancb | Itm2b |
| Gm14412 | Wfdc2 | Zfp119b | Tgfbr2 | Lrp11 | Sdcbp2 | Rac3 | Dusp11 |
| Otx2 | Msx2 | X1700086P04Rik | Dmc1 | Trim21 | Fam132a | Mthfsd | Lgals9 |
| NA.1866 | X5730507C01Rik | Csta1 | Ctgf | Slc24a5 | X2700068H02Rik | Acadvl | Sdhaf4 |
| Oxt | Herpud1 | Efnb1 | Sult4a1 | Csf3r | Kbtbd13 | NA.10404 | Emp2 |
| BC051142 | Hspa1b | Hcmk1 | Zfp459 | 43352 | NA.102 | Tfcp2l1 | Idh1 |
| Kcnn4 | Adamts10 | X4930522L14Rik | Zfp688 | Lrrc75b | Gimap9 | NA.1896 | Zfp850 |
| Zfp931 | Mdh1 | Hormad2 | Cgref1 | NA.13142 | Gm4262 | X1010001B22Rik | Txndc17 |
| Plet1 | Rhoc | Cd82 | NA.92 | Map2k3os | NA.6479 | Erf | Apeh |
| Ppl | Ier2 | Map3k1 | Naa11 | Prkce | Ralb | Slc28a3 | Gm10439 |
| Chpf | Slfn3 | X1500009L16Rik | NA.388 | X4930563D23Rik | Tmem17 | Junb | NA.1925 |
| Tspan3 | Zfp759 | Phf11d | Tdrp | Ank | NA.1999 | Zfp119a | Cnn3 |
| Hyal2 | B3galt2 | NA.13623 | Pcbd1 | Dact2 | Leprot | Perp | Mmp15 |
| Fstl3 | Lacc1 | Trim38 | Slco2a1 | Pacsin3 | Ube2q2 | NA.369 | Cxcr6 |
| Slfn2 | Tns1 | Vps29 | Cyb5r1 | Hmcn2 | Lmf1 | Calcoco2 | Foxb2 |
| Dusp6 | Tmem45b | Tbl1x | Magea2 | Eef2kmt | Tmem147 | Gm28085 | Lama5 |
| Cat | Tap1 | Lsr | Prokr1 | Chchd7 | Sh3bgrl3 | C1qa | X1700080O16Rik |
| Nppb | Slc38a4 | Il17rc | Mbnl2 | Zfp248 | Tradd | NA.1618 | Gm16136 |
| Tpcn2 | D10Jhu81e | Aqp3 | Mex3b | NA.10780 | Il10rb | Zfp81 | Asap3 |
| Ccdc169 | Srxn1 | Zfp429 | Gm16712 | Clec11a | X1700086O06Rik | Ntf5 | Syngr4 |
| Elovl5 | Spata9 | Ggt1 | Zfp395 | Sgl1 | Sdhaf3 | Oas1g | Zdhhc15 |
| NA.12239 | Pmepa1 | Tcea2 | Krt8 | Xlr3a | Galnt9 | Appl2 | Fam83b |
| Zfp326 | Gm26853 | Gm5141 | Tceal1 | Msc | Ogdhl | Gna15 | Rnase4 |
| AI317395 | Pfkfb4 | Tmem51 | Gata3 | Zfp442 | Pear1 | Gm6169 | Fbxl21 |
| AA467197 | Zfp266 | Stx7 | Scrinc2 | Gm14418 | Fezf1 | Cma1 | Hdx |
| NA.113 | Cdc42ep5 | A530017D24Rik | Rgs14 | Usp25 | Svbp | Lrrn2 | |
| 35 | Magea3 | X1700003M07Rik | Mocs1 | Ntpcr | Larp1b | Acot6 | |
| Ptges | Chrna3 | Lad1 | Tmem131 | Pros1 | A730015C16Rik | Dmrta2 | |
| Smim1 | Gm26624 | Hint2 | Vps45 | Lpp | Gm26779 | Skida1 | |
| Kirrel | Elovl7 | Exph5 | Plpp2 | Trp53i11 | Cryzl1 | Ccng1 | |
| Gbp9 | Nkx6.2 | Sfrp1 | Mogat2 | X2610008E11Rik | St14 | Trabd | |
| Ckap4 | Crtam | Hspe1 | NA.12035 | Akr1e1 | Egr4 | X2410022M11Rik | |
| Napsa | Nfkbiz | X9430065F17Rik | B230118H07Rik | Pla2g7 | Hmga1.rs1 | Tet2 | |
| Gjb5 | Cyp4f14 | Ahcy | Serpinb6c | NA.4703 | Lcp1 | Cetn3 | |
| Clic3 | Tnfrsf1b | Magee2 | Fos | Gmpr2 | Hadh | Sri | |
| Marcks | Dsp | Mageb4 | P2ry2 | Stard10 | Sec14l4 | Vill | |
| NA.7249 | Khnayn | Gm7325 | Lgals4 | Enpep | Txndc12 | Msantd4 | |
| Scd2 | Rnd1 | Tmem266 | Epb41l1 | Prss35 | NA.7425 | Abhd14a | |
| Adgre5 | Hnf4a | Txn1 | Snrk | NA.2001 | Hist1h2bc | Gm4131 | |
| Fam129b | Adat2 | Rec8 | X2410018L13Rik | Eml2 | P2rx3 | Pnpla6 | |
| Pycr2 | X2200002D01Rik | Tgm2 | Rims4 | Ggdc | Arhgef5 | NA.4131 | |
| Dcaf12l1 | Gabarapl1 | Xkr6 | Gchfr | X2610301B20Rik | Sfmbt2 | Smap1 | |
| Barx2 | NA.12352 | Egln3 | Nrg1 | Pdzd3 | Btg2 | Lysmd2 | |
| Il4ra | Shc2 | Man2a1 | Skil | Gm5424 | Ndufc2 | Xrcc4 | |

32-cell

| | | | | | | | |
|---|---|---|---|---|---|---|---|
| Lrp2 | Ezr | Oc90 | Ptpm | Baiap2l1 | Plod2 | Tcn2 | Fez2 |
| Fhl2 | Fam213b | Mapre3 | Gpr4 | Cdc42ep5 | Phf11d | Rnaset2b | Rap2b |
| Capn2 | Xbp1 | Gm364 | Ptgr1 | Etfb | Pdgfa | Aldh2 | Prkce |
| Spp1 | Ceacam10 | Gsto1l | 43352 | Gm12169 | S100a10 | Dab2ip | Gm2381 |
| BC053393 | NA.5461 | Nanog | Nrl | Mdh1 | Tpm4 | Actb | Gucy1b2 |
| Hspb8 | Msn | Eml2 | Optn | Plet1 | Pgm2 | Cck | NA.7242 |
| Cdx2 | Frmd4b | Lsr | Slc25a13 | Wdr1 | Gm14326 | Efhd2 | Hist1h1e |
| Krt18 | Glrx | St14 | Dqx1 | Zfp37 | Xrcc5 | Pank4 | Gmpr |
| Enpep | Gapdh | Nfic | GM26579 | Hist1h3c | Esd | Arvcf | Pla2g6 |
| Elf3 | Gstp1 | B230118H07Rik | Tmem125 | H2afy | Actr3b | GM14327 | NA.2972 |
| Vgll3 | Serpinb6c | Gm6169 | Cmip | NA.148 | D630003M21Rik | Wdr6 | NA.7262 |
| Wnt7b | Epb41l1 | GM7325 | Gm14325 | X1700042G15Rik | Ppp1r14d | Abcg2 | Anxa6 |
| Akr1b8 | NA.12312 | Gm26917 | Dtd2 | Adrb3 | Mkrn3 | Mgst1 | Fthl17e |
| C2cd4a | Lgals1 | Zfp931 | Tspan3 | Gm14399 | Adgrl2 | Aldh3a2 | Cdc42ep3 |
| Bglap3 | Ptges | Rp2 | Srxn1 | Fthl17a | NA.10114 | Omd | Tradd |
| Rab17 | D10Jhu81e | Tat | Hus1b | H2.D1 | Sox6 | Chrna1 | Sccpdh |
| Serpinb9b | Stard10 | Epcam | Slc6a13 | Cat | Tns1 | Tdp1 | Xlr3b |
| Bmyc | Apoa1 | Rnf130 | Adam15 | NA.1550 | Emp2 | Sgpl1 | Figla |
| Cmbl | Cela2a | Gm14403 | Vill | Fgfbp1 | Col4a1 | Ttf2 | NA.14180 |
| Klf6 | Tuba4a | Tmem139 | Sult6b1 | Lgals4 | Ndrg1 | Fam129b | Dap |
| Krt8 | H2.K1 | Pycr2 | Mecp2 | Trim50 | Dap3 | Emc9 | Hspd1 |
| Nppb | Hint2 | Plscr1 | Tarm1 | Prkcdbp | Capzb | Tmem17 | Efcab10 |
| Tpp1 | Cubn | Mfi2 | Camk1 | Trpm6 | Fhl4 | NA.102 | Tubb2a |
| Tmem9 | Rnf128 | Adad2 | Mgl2 | NA.1546 | Wfdc2 | Vps29 | Gprc5d |
| Dppa1 | Dusp4 | Dsp | Chst13 | Cidea | Anp32a | AU021092 | Smim12 |
| Rhox5 | Ogdhl | Mbp | Myh13 | Nagk | X2310015A10Rik | Pard6g | Mtmr7 |
| Gm5424 | X1500009L16Rik | Chrnb4 | Barx2 | Slc38a4 | Hist3h2a | Kcnk12 | Gsta3 |
| Id2 | Tet2 | Tfcp2l1 | X1810030O07Rik | Serinc2 | Slc37a2 | X8030474K03Rik | Skida1 |
| Gjb5 | Chmp2b | Exph5 | Ccdc43 | Rgs14 | Gm14418 | Atp1b1 | Idh1 |
| Nek6 | Lama3 | Rcan1 | Ppm1m | Tpi1 | Hsd17b4 | A330050F15Rik | Hlf |
| Oas1a | Fbxo3 | X9530059O14Rik | Slc24a5 | Gstz1 | Sergef | Hdac3 | Tcea3 |
| Scd2 | Elovl7 | Eef2kmt | Xlr3a | Ggt1 | Psme2b | Ftx | Znrd1as |
| Atp12a | Patl2 | Muc1 | Tmem198 | Insig2 | Il11ra1 | Fthl17d | Pkm |
| Gstp2 | Ccdc13 | Efcab5 | BC051019 | Ly6a | Tpcn2 | NA.4386 | Map3k15 |
| Ngfrap1 | Col4a2 | Nynrin | Erbb2 | X2310039H08Rik | Sh3bgrl2 | Arl2 | Ak4 |
| Pycard | Acaa2 | Gm26603 | Cnpy2 | NA.2957 | Asic3 | Apeh | Gm12828 |
| Pafah2 | Acaa1a | Nlrp4c | Idh3a | Car12 | Lurap1l | Slc2a12 | Myole |
| Csta1 | Apbb1ip | Susd2 | Dab2 | F2rl1 | Plau | Zfp850 | Slc4a5 |
| Fam213a | Tmx4 | Tst | Mks1 | Zfp454 | Fam83h | Ift140 | Slc2a3 |
| Bin1 | Snai2 | Khdc3 | Gimap9 | Eci3 | Trp53i11 | Slc2a1 | Sdr42e1 |
| Gm694 | AI662270 | Plb1 | NA.1892 | Gjb3 | AA467197 | Prkx | Slc7a6 |
| Dsg2 | Sox9 | NA.5999 | Hk2 | Ly6f | Gm14409 | X1700086O06Rik | Snx19 |
| Ass1 | Tes | Tdrp | Marcks | Pnliprp2 | NA.513 | Cox7b | Ndufaf3 |
| Gm4737 | Trim38 | Gale | Gm773 | Praf2 | Mettl7a1 | Fam136a | Plin2 |
| Slc38a1 | Cryz | GM14322 | NA.83 | Gm14393 | Clic4 | Pwwp2b | Gipc1 |
| Slc38a11 | Anxa2 | Cpxm1 | Cdk5 | Abcb8 | Acol | Cyb5r3 | Pla2g4f |
| Camk2d | Sft2d2 | Tmprss12 | Gstm6 | Mras | Sh3bp5 | Mapt | |
| Bex2 | NA.388 | S100a11 | Atxn10 | Gm14444 | NA.1866 | Vps13c | |
| Sdc4 | X2610528J11Rik | Hoxd3os1 | Smco2 | Bckdhb | GM4779 | Abca1 | |
| Rfx4 | Gsn | A230005M16Rik | Eno1b | NA.9436 | Cbr4 | Hibch | |
| NA.7440 | Hadh | Hnf4a | Pir | Tbx15 | NA.6249 | Mical1 | |
| Tinagl1 | X0610009O20Rik | Hist1h3d | Gpx2 | Acsf2 | Myh10 | Adat2 | |
| Col7a1 | Plp2 | Bdnf | Csf3r | Slc18a1 | Crip2 | Lpp | |
| Kng2 | Abcc4 | Ppp4r1 | Atg4c | Hdx | Psmb9 | Srebf1 | |
| Adgre5 | Lcp1 | Lta4h | Uhrf1 | Apoc1 | Gm4926 | Arhgap9 | |
| Tnftsf9 | Actg1 | Dpysl4 | Clic3 | Serpinb6a | Il17rc | NA.14050 | |
| Mmel1 | Fam25c | Tmem102 | Gstm7 | Zyx | Sdhaf4 | Tctn1 | |
| Lgals9 | Xk | Trhr2 | Coasy | Rec8 | Dok1 | Tuba1b | |
| Tex19.2 | NA.92 | Tbl1x | Tmem256 | Ppp1r18 | Slc25a39 | Whamm | |
| Gata3 | Fabp3.ps1 | Kremen2 | NA.529 | Cyb5a | Ccdc42 | Smyd4 | |
| Atxn7l1 | Ube2l6 | D130040H23Rik | Tmem45b | Fbln1 | Atp8a1 | Cbfa2t3 | |
| Txndc12 | Nsmaf | Cyp4f39 | Krt23 | Dpy19l1 | Echs1 | Arhgef25 | |
| Clcnkb | Cited4 | Tmem266 | Mpzl2 | Tpm1 | Akr1e1 | Nbl1 | |
| Trp53bp2 | Fabp3 | NA.5910 | Sqstm1 | Gdfi | Nudt11 | Mgat4b | |
| | As3mt | Gss | Zfp780b | Map2k6 | Gcat | Adh4 | |

In a nutshell, and further discussed below, we identified notable features within the landscape, including sets of cells classified as pluripotent-, epithelial-, trophoblast-, neural-, and stromal-like based on strong expression of signatures related to these cell types and a set of cells (FIG. 24E, purple) that appeared poised to undergo a mesenchymal-to-epithelial transition (MET) following withdrawal of dox (FIG. 24E, orange). The relative proportions of these subsets at different times differed between serum and 2i conditions (FIG. 24G).

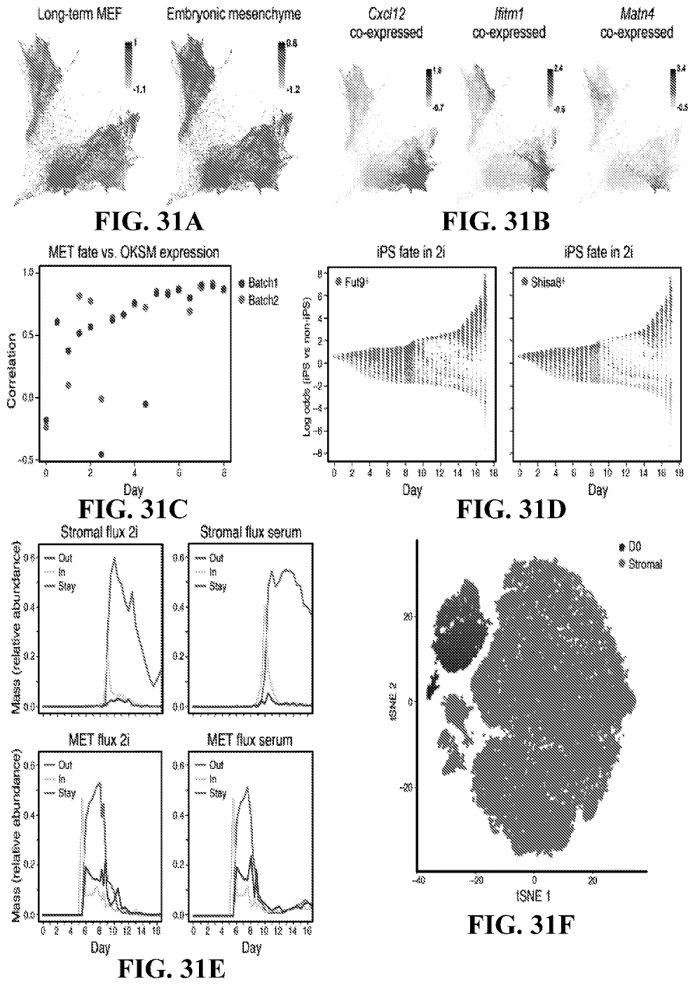

Using Waddington-OT, we calculated the ancestor and descendant distributions for all cells and determined the trajectories to/from various cell sets (FIG. 24F, arrows). Briefly, the time course began with MEFs at day 0 in the lower right, proceeded leftward to day 2, and then upward over the subsequent week toward two destinations: the MET Region and the Stromal Region. The cells in the MET Region were predicted to give rise to the pluripotent-, epithelial-, trophoblast-, and neural-like cells, with this last class seen in serum but not 2i conditions. By contrast, the Stromal Region appeared to be terminal: cells entered the region, but our model predicted that they did not leave (FIG. 31E).

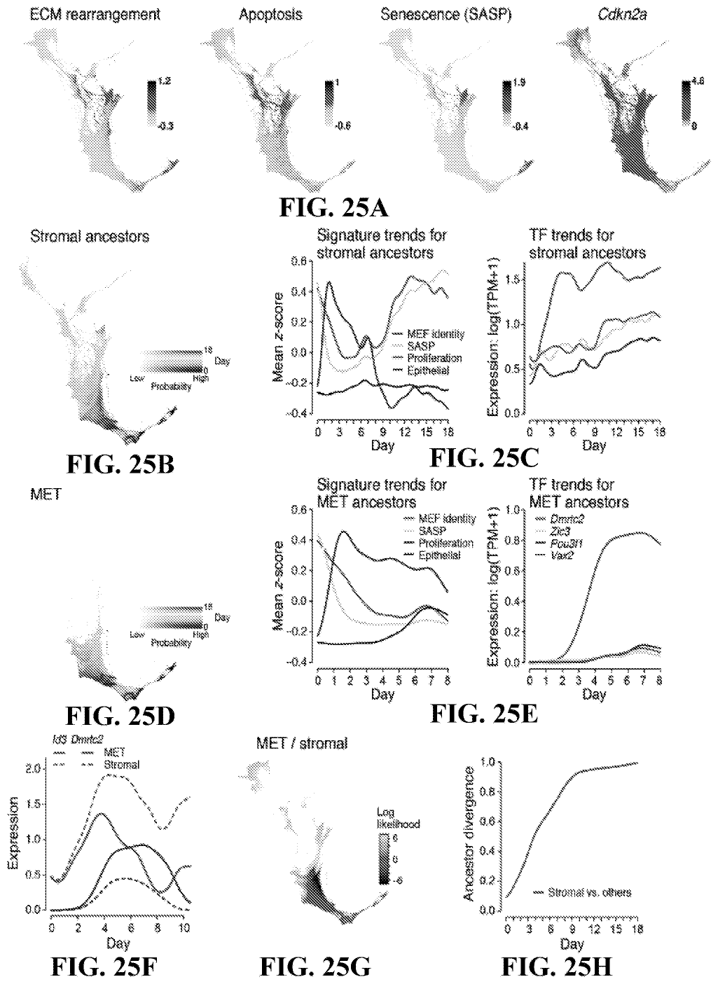

The optimal-transport analysis provided insights into when cell fates emerged. As early as 1.5 days, cells' fates began to concentrate toward either the MET Region or Stromal Region, and the distinction sharpened over the next several days (FIG. 25G). The fate of pluripotent-, epithelial-, trophoblast-, and neural-like cells did not appear to be determined until after withdrawal of dox on day 8. That was, the ancestor distributions of these cell types were indistinguishable on and before day 8.

The Model was Predictive and Robust

Before analyzing the cell sets and trajectories in greater detail, we assessed the accuracy and robustness of our model. Because current experimental approaches for tracing cell lineage did not provide a rich description of the full transcriptional state of a cell set's ancestors, we developed a computational approach to test the model. Specifically, we used optimal transport between the distribution of cells at times t1 and t3 to predict the distribution of cells at an intermediate time t2 and compared this prediction to the observed distribution at t2.

Our predicted trajectories were accurate, such that the distance between the computational prediction and experimental observation at t2 was similar in magnitude to the distance between the two experimental replicates taken at t2, confirming that the prediction is roughly as good as could be expected given experimental variation (FIG. 24H, FIGS. 30A-30G, Methods).

The optimal-transport analysis was also robust to perturbations of the data and parameter settings. We down-sampled the number of cells at each time point, down-sampled the number of reads in each cell, perturbed our initial estimates for cellular growth and death rates, and perturbed the parameters for entropic regularization and unbalanced transport. In all cases, we found that the interpolation results above are stable across wide range of perturbations (STAR Methods).

In initial stages of reprogramming, cells progressed toward stromal or MET fates

Reprogramming began with all cells exhibiting rapid changes. By day 1, cells showed an increase in cell-cycle signatures and a decrease in MEF identity. MEF identity continued to fall through day 3, by which point nearly all cells showed lower signatures than the vast majority of MEFs at day 0 (FIG. 24D). Over time, cells assumed either Stromal or MET identities (FIGS. 25A-25H).

Cells in the Stromal Region showed distinctive signatures, which fully emerged after withdrawal of dox at day 8; these signatures included a secretory phenotype (SASP), extracellular matrix (ECM) rearrangement, senescence, and cell cycle inhibitors (FIG. 25A). By contrast, the MET Region contained cells with increased proliferation and loss of fibroblast identity (FIG. 25E).

Mapping signatures of distinct stromal cell types obtained across mouse tissues from a mouse cell atlas (Han et al., 2018) showed that the most widely expressed stromal signatures corresponded to embryonic mesenchyme and long-term cultured MEFs (FIG. 31A). Yet, the Stromal Region did not simply reflect “MEF reversion.” The gene expression profiles were distinct from (FIG. 31F) and more heterogeneous than day 0 MEFs, with clusters of cells with signatures that more closely correspond to other stromal cell types, such as those found in neonatal muscle and neonatal skin (p-values<0.01) at levels 20- to 30-fold higher than day 0 MEFs.

The proportion of stromal cells peaks several days after dox withdrawal (at ˜64% of cells at day 10.5 in 2i conditions and day 11 in serum conditions) and then declines through day 18, consistent with the low proliferation signature relative to other cells in the landscape (FIG. 24G). A subset of stromal cells expresses an apoptosis signature starting on day 9, which peaks at day 14.5 in ˜14% of stromal cells in serum conditions and at day 13 in ˜3% in 2i conditions.

Our trajectory analysis allowed us to trace how these fates were gradually established: we found that the ancestor distributions of cells in the Stromal and MET Regions differred by 30% at day 3 and by 60% at day 6 (FIG. 25H). A powerful predictor of a cell's fate was its expression level of the OKSM transgene, with high values predictive of MET fate and low values predictive of stromal fate (FIG. 31C); the expression level statistically explained ˜50% of the variance in the logarithm of the fate ratio (MET Region fate probability divided by Stromal Region fate probability) by day 2 and ˜75% by day 5 (FIG. 31C). Importantly, the divergence was gradual and could not be described by a simple graph with a sharp (that was, zero-dimensional) branch point. Indeed, our optimal-transport analysis indicated that a significant minority of cells that were on the trajectory to the MET region continues to switch to the trajectory to the Stromal Region (FIG. 25G).

Regulatory analysis identified TFs associated with the two trajectories. Three TFs (Dmrtc2, Zic3, and Pou3f1) were induced in all cells (from undetectable levels at day 0), but showed higher expression along the trajectory to the MET Region (FIG. 25E, 25F). Zic3 was required for maintenance of pluripotency (Lim et al., 2007), Pou3f1 was required for self-renewal of spermatogonial stem cells (Wu et al., 2010), and Dmrtc2 was involved in germ cell development (Gegenschatz-Schmid et al., 2017; Yamamizu et al., 2016). Four TFs (Id3, Nfix, Nfic, and Prrx1) were upregulated in all cells (from basal levels at day 0) but showed higher expression in cells with a stromal fate (FIGS. 25E, 25F). (Analysis of subsequent time points showed that, following withdrawal of dox, these genes maintained high expression in stromal cells but shut off in cells along the trajectory to iPSCs.) Nfix was reported to repress embryonic expression programs in early development, while Nfic and Prrx1 were associated with mesenchymal programs (Froidure et al., 2016; Messina et al., 2010; Ocana et al., 2012). Id3 was known to inhibit transcription through formation of nonfunctional dimers that were incapable of binding to DNA. Higher expression of Id3 along the trajectory toward stromal cells may seem somewhat surprising, because forced expression of Id3 was shown to increase reprogramming efficiency (Hayashi et al., 2016; Liu et al., 2015). However, Id3 might cause increased efficiency via its activity in stromal cells, which secreted factors that enhance iPSC reprogramming (Mosteiro et al., 2016) (see below), or via activity in non-stromal cells, in which it was expressed through day 8, albeit at lower levels.

There has been much interest in finding early markers of successful reprogramming-namely, genes whose early expression was correlated with a cell's descendants being enriched for iPSCs. Our analysis suggested that it would be more precise to define “early markers of successful MET”, because the iPSC, trophoblast and neural fates did not appear to be established until after withdrawal of dox at day 8.

Trajectory analysis revealed early markers of successful MET, including known markers such as Fut9 (which synthesizes the glyco-antigen SSEA-1) and novel candidates such as Shisa8. Shisa8 was the most differentially expressed gene at day 1.5. When we sorted cells based on the ratio of their likelihood of transition to the MET Region vs Stromal Region, we found Shisa8 expressed in 50% of the top quartile but only 5% of cells in the bottom quartile. (Table 16). Shisa8 was a little-studied mammalian-specific member of the Shisa gene family in vertebrates, which encoded single-transmembrane proteins that played roles in development and are thought to serve as adaptor proteins (Pei and Grishin, 2012; Polo et al., 2012). (Analysis of subsequent time points showed that Shisa8 and Fut9 also showed similar patterns following dox withdrawal: both were expressed strongly in cells along the trajectory toward successful reprogramming, and lowly expressed in other lineages (FIG. 31D).)

TABLE 16
Differential genes between top ancestors of MET vs. top ancestors of stromal cells.
Differential genes between top ancestors of MET vs. Stromal cells at D1.5

| | | | Fraction | Fraction | |
|---|---|---|---|---|---|
| | | | expressed in | expressed in | |
| | | Average | top ancestors | top ancestors | Adjusted |
| Gene | p-value | logFC | of MET | of stromal cells | p-value |
| Shisa8 | 2.37E−56 | 0.439583976 | 0.505 | 0.051 | 4.52E−52 |
| Anpep | 1.24E−44 | 0.399501581 | 0.548 | 0.141 | 2.37E−40 |
| Gch1 | 5.09E−37 | 0.381008072 | 0.607 | 0.245 | 9.71E−33 |
| Gpm6b | 1.24E−29 | 0.275486032 | 0.538 | 0.209 | 2.37E−25 |
| Npnt | 3.61E−30 | 0.382743398 | 0.714 | 0.395 | 6.89E−26 |
| Dsp | 9.36E−34 | 0.290320422 | 0.389 | 0.072 | 1.79E−29 |
| Rb1 | 1.12E−25 | 0.280506707 | 0.616 | 0.315 | 2.13E−21 |
| Dgat2 | 5.18E−28 | 0.349298687 | 0.524 | 0.225 | 9.88E−24 |
| Car12 | 1.06E−23 | 0.299588702 | 0.552 | 0.254 | 2.02E−19 |
| Lrp4 | 9.73E−27 | 0.247967802 | 0.405 | 0.11 | 1.86E−22 |
| C1ql3 | 2.93E−26 | 0.325323868 | 0.45 | 0.155 | 5.60E−22 |
| Sgol2a | 1.65E−25 | 0.33023125 | 0.685 | 0.395 | 3.16E−21 |
| Gm26737 | 2.93E−25 | 0.534938533 | 0.656 | 0.368 | 5.59E−21 |
| Lepr | 1.15E−22 | 0.588193067 | 0.695 | 0.417 | 2.19E−18 |
| Nol4l | 1.78E−21 | 0.374175462 | 0.65 | 0.374 | 3.40E−17 |
| Gm29666 | 1.49E−20 | 0.279383915 | 0.511 | 0.237 | 2.84E−16 |
| Pfkp | 8.34E−30 | 0.316216243 | 0.796 | 0.524 | 1.59E−25 |
| RP23-4H17.3 | 4.98E−21 | 0.441940336 | 0.695 | 0.425 | 9.51E−17 |
| Ralgps2 | 4.40E−22 | 0.217741022 | 0.38 | 0.117 | 8.40E−18 |
| Xaf1 | 1.12E−18 | 0.328905337 | 0.564 | 0.307 | 2.14E−14 |
| Zdhhc2 | 2.08E−17 | 0.200585787 | 0.519 | 0.264 | 3.97E−13 |
| Ppm1k | 1.38E−22 | 0.307219164 | 0.658 | 0.411 | 2.63E−18 |
| Mcm10 | 1.99E−16 | 0.230302782 | 0.593 | 0.348 | 3.80E−12 |
| Gm13075 | 1.33E−27 | 0.861118262 | 0.771 | 0.528 | 2.53E−23 |
| Rep15 | 2.80E−18 | 0.29626083 | 0.658 | 0.423 | 5.34E−14 |
| Pola2 | 3.37E−23 | 0.311939681 | 0.748 | 0.519 | 6.44E−19 |
| Trim37 | 7.52E−17 | 0.218079056 | 0.583 | 0.358 | 1.44E−12 |
| Rtkn | 3.27E−18 | 0.287996995 | 0.382 | 0.16 | 6.24E−14 |
| Ppif | 1.58E−21 | 0.252798031 | 0.767 | 0.548 | 3.02E−17 |
| Rsf1 | 2.84E−15 | 0.229977128 | 0.591 | 0.374 | 5.42E−11 |
| Ptcra | 5.85E−13 | 0.417578437 | 0.413 | 0.2 | 1.12E−08 |
| Nmrk1 | 4.51E−13 | 0.528279491 | 0.554 | 0.344 | 8.61E−09 |
| Perp | 4.55E−65 | 0.656396496 | 0.963 | 0.753 | 8.69E−61 |
| Chmp2b | 1.29E−30 | 0.335057338 | 0.849 | 0.64 | 2.46E−26 |
| Pcgf2 | 5.58E−15 | 0.541239697 | 0.591 | 0.387 | 1.07E−10 |
| Gmcl1 | 4.30E−14 | 0.523834071 | 0.544 | 0.344 | 8.21E−10 |
| Pacs1 | 1.50E−18 | 0.251074727 | 0.785 | 0.587 | 2.87E−14 |
| Wdr35 | 3.75E−14 | 0.224471336 | 0.656 | 0.464 | 7.15E−10 |
| Ppat | 2.16E−16 | 0.243243284 | 0.708 | 0.517 | 4.13E−12 |
| Slamf1 | 5.19E−11 | 0.228267013 | 0.468 | 0.28 | 9.90E−07 |
| Homer2 | 6.66E−14 | 0.236094482 | 0.624 | 0.438 | 1.27E−09 |
| Cenph | 7.86E−14 | 0.206088745 | 0.72 | 0.538 | 1.50E−09 |
| B930036N10Rik | 2.34E−10 | 0.518225771 | 0.544 | 0.368 | 4.46E−06 |
| Hpcal1 | 8.65E−13 | 0.208476389 | 0.613 | 0.438 | 1.65E−08 |
| H2-T23 | 8.64E−11 | 0.235054556 | 0.337 | 0.164 | 1.65E−06 |
| Sgol1 | 2.01E−16 | 0.266408936 | 0.853 | 0.683 | 3.83E−12 |
| Ccdc137 | 2.58E−20 | 0.287870449 | 0.793 | 0.624 | 4.93E−16 |
| Exosc2 | 9.42E−37 | 0.652481854 | 0.933 | 0.765 | 1.80E−32 |
| Gkap1 | 1.74E−23 | 0.397791708 | 0.781 | 0.613 | 3.31E−19 |
| Agl | 1.58E−16 | 0.495744367 | 0.798 | 0.63 | 3.01E−12 |
| Ckap2 | 8.06E−12 | 0.205735226 | 0.796 | 0.632 | 1.54E−07 |
| Nt5dc3 | 1.29E−10 | 0.200909668 | 0.638 | 0.481 | 2.46E−06 |
| Tapbpl | 7.86E−09 | 0.226071905 | 0.315 | 0.164 | 0.000150089 |
| Shoc2 | 9.21E−15 | 0.231434184 | 0.751 | 0.601 | 1.76E−10 |
| Faap24 | 3.98E−11 | 0.2159197 | 0.642 | 0.495 | 7.60E−07 |
| Haus8 | 2.63E−16 | 0.634579918 | 0.744 | 0.599 | 5.01E−12 |
| Cenpf | 7.61E−11 | 0.214446511 | 0.908 | 0.763 | 1.45E−06 |
| Mrps11 | 3.66E−41 | 0.430516438 | 0.906 | 0.763 | 6.99E−37 |
| Aldh3a1 | 8.14E−08 | 0.221022512 | 0.456 | 0.313 | 0.001554728 |
| Gm7120 | 8.12E−08 | 0.306764672 | 0.311 | 0.168 | 0.001550761 |
| Lpgat1 | 4.28E−16 | 0.244225687 | 0.806 | 0.665 | 8.17E−12 |
| Topbp1 | 5.86E−12 | 0.224664357 | 0.734 | 0.593 | 1.12E−07 |
| Mrps6 | 3.39E−43 | 0.396132536 | 0.939 | 0.798 | 6.47E−39 |
| 1700047l17Rik2 | 5.69E−09 | 0.200128893 | 0.521 | 0.382 | 0.000108639 |
| Myc | 4.08E−26 | 0.347729368 | 0.898 | 0.763 | 7.80E−22 |
| Timm10 | 4.34E−14 | 0.223178202 | 0.845 | 0.71 | 8.28E−10 |
| Mrpl9 | 9.74E−09 | 0.222293218 | 0.503 | 0.368 | 0.000185972 |
| Fam114a2 | 2.19E−18 | 0.23879583 | 0.83 | 0.697 | 4.18E−14 |
| Rrn3 | 1.49E−11 | 0.228168673 | 0.724 | 0.591 | 2.84E−07 |
| Dcaf17 | 2.63E−08 | 0.521823548 | 0.487 | 0.354 | 0.00050265 |
| Asph | 2.31E−14 | 0.224904909 | 0.787 | 0.656 | 4.42E−10 |
| Abcb1b | 6.60E−40 | 0.441369564 | 0.947 | 0.818 | 1.26E−35 |
| Ctnnbl1 | 2.19E−11 | 0.207192935 | 0.777 | 0.648 | 4.18E−07 |
| Slbp | 1.84E−15 | 0.374861946 | 0.873 | 0.748 | 3.52E−11 |
| Tex10 | 3.22E−15 | 0.251420666 | 0.8 | 0.677 | 6.14E−11 |
| Dennd5b | 3.94E−11 | 0.298384346 | 0.755 | 0.632 | 7.52E−07 |
| Lrrc42 | 3.19E−14 | 0.250507008 | 0.748 | 0.626 | 6.09E−10 |
| Paip2b | 6.60E−09 | 0.233070859 | 0.691 | 0.571 | 0.000126059 |
| 1700037H04Rik | 3.73E−13 | 0.21591323 | 0.777 | 0.663 | 7.12E−09 |
| Noa1 | 1.13E−34 | 0.490924229 | 0.9 | 0.787 | 2.17E−30 |
| Gtf2h1 | 5.71E−19 | 0.253937461 | 0.843 | 0.738 | 1.09E−14 |
| Ndc1 | 4.28E−18 | 0.25208573 | 0.89 | 0.785 | 8.16E−14 |
| Ddx42 | 1.64E−13 | 0.213024231 | 0.83 | 0.726 | 3.13E−09 |
| Golga3 | 9.43E−07 | 0.495832978 | 0.595 | 0.491 | 0.018003133 |
| Pop5 | 1.28E−28 | 0.301595886 | 0.949 | 0.847 | 2.44E−24 |
| Tgfbi | 1.63E−09 | 0.200070657 | 0.828 | 0.726 | 3.11E−05 |
| Hells | 3.70E−13 | 0.222587886 | 0.949 | 0.851 | 7.06E−09 |
| Plk4 | 1.42E−23 | 0.57479234 | 0.922 | 0.826 | 2.72E−19 |
| Ezh2 | 1.90E−18 | 0.236909466 | 0.906 | 0.81 | 3.64E−14 |
| Naa20 | 8.41E−18 | 0.270587809 | 0.806 | 0.714 | 1.61E−13 |
| Epn1 | 1.54E−14 | 0.209191303 | 0.902 | 0.812 | 2.94E−10 |
| Smn1 | 9.92E−38 | 0.401700379 | 0.941 | 0.853 | 1.89E−33 |
| Mcm7 | 1.42E−16 | 0.229113377 | 0.955 | 0.867 | 2.72E−12 |
| Enah | 1.19E−12 | 0.207086155 | 0.828 | 0.742 | 2.27E−08 |
| Mrps25 | 2.24E−16 | 0.238478878 | 0.863 | 0.783 | 4.27E−12 |
| Carnmt1 | 7.08E−15 | 0.213768504 | 0.871 | 0.791 | 1.35E−10 |
| Zfp106 | 4.55E−12 | 0.206955912 | 0.943 | 0.863 | 8.69E−08 |
| Hmgb3 | 4.37E−16 | 0.244565953 | 0.879 | 0.802 | 8.34E−12 |
| Psmb10 | 8.45E−25 | 0.305887579 | 0.937 | 0.861 | 1.61E−20 |
| Scp2 | 7.16E−12 | 0.211532788 | 0.883 | 0.808 | 1.37E−07 |
| Hist1h2ap | 1.60E−27 | 0.599321987 | 0.978 | 0.904 | 3.05E−23 |
| Limk2 | 1.79E−12 | 0.34639987 | 0.81 | 0.738 | 3.42E−08 |
| Dbf4 | 5.21E−15 | 0.209332579 | 0.922 | 0.851 | 9.95E−11 |
| Baz1a | 2.09E−20 | 0.276857187 | 0.881 | 0.812 | 4.00E−16 |
| Ifrd2 | 4.47E−21 | 0.25780276 | 0.908 | 0.84 | 8.53E−17 |
| Ccdc50 | 1.00E−25 | 0.293196782 | 0.955 | 0.888 | 1.92E−21 |
| Pbdc1 | 3.94E−14 | 0.228782894 | 0.875 | 0.808 | 7.52E−10 |
| Wdr45b | 8.91E−11 | 0.203638926 | 0.832 | 0.769 | 1.70E−06 |
| Noc2l | 8.02E−21 | 0.235002625 | 0.951 | 0.89 | 1.53E−16 |
| Ruvbl1 | 3.88E−11 | 0.20097654 | 0.828 | 0.767 | 7.41E−07 |
| Prmt5 | 1.96E−13 | 0.20762784 | 0.888 | 0.832 | 3.74E−09 |
| Tmem245 | 1.26E−32 | 0.731436804 | 0.963 | 0.908 | 2.40E−28 |
| Pno1 | 1.18E−22 | 0.284205102 | 0.894 | 0.84 | 2.25E−18 |
| Chchd7 | 1.97E−33 | 0.376522958 | 0.92 | 0.867 | 3.76E−29 |
| Yif1b | 2.51E−12 | 0.204286063 | 0.91 | 0.857 | 4.80E−08 |
| Nip7 | 1.61E−09 | 0.317643192 | 0.896 | 0.843 | 3.07E−05 |
| Stmn1 | 7.91E−13 | 0.214767905 | 0.926 | 0.875 | 1.51E−08 |
| Rtcb | 3.23E−21 | 0.248019171 | 0.933 | 0.885 | 6.16E−17 |
| Nmt2 | 9.69E−54 | 0.59549564 | 0.988 | 0.941 | 1.85E−49 |
| Fnta | 2.30E−11 | 0.208830016 | 0.824 | 0.779 | 4.40E−07 |
| Snhg9 | 4.41E−41 | 0.578853339 | 0.971 | 0.928 | 8.42E−37 |
| Tax1bp1 | 1.04E−11 | 0.20563376 | 0.855 | 0.812 | 1.98E−07 |
| Cdk6 | 9.45E−13 | 0.216050004 | 0.935 | 0.896 | 1.80E−08 |
| Tcof1 | 3.45E−31 | 0.302647593 | 0.965 | 0.928 | 6.58E−27 |
| Cebpz | 1.09E−16 | 0.237798069 | 0.939 | 0.902 | 2.09E−12 |
| Loxl2 | 1.30E−17 | 0.571139295 | 0.89 | 0.857 | 2.48E−13 |
| Rangap1 | 2.34E−40 | 0.369409656 | 0.984 | 0.953 | 4.46E−36 |
| Dek | 1.64E−18 | 0.231074803 | 0.996 | 0.967 | 3.12E−14 |
| Nolc1 | 9.61E−30 | 0.309060428 | 0.986 | 0.959 | 1.83E−25 |
| Mybbp1a | 1.01E−15 | 0.209760443 | 0.969 | 0.943 | 1.92E−11 |
| Uchl3 | 4.63E−23 | 0.291386824 | 0.963 | 0.937 | 8.83E−19 |
| Mt2 | 2.21E−46 | 0.647830277 | 0.982 | 0.959 | 4.21E−42 |
| Fam177a | 7.40E−29 | 0.318947806 | 0.965 | 0.943 | 1.41E−24 |
| Ak2 | 2.85E−38 | 0.322110667 | 0.992 | 0.971 | 5.45E−34 |
| Pdcd11 | 1.06E−26 | 0.317776644 | 0.994 | 0.973 | 2.03E−22 |
| Clns1a | 7.78E−15 | 0.200963226 | 0.955 | 0.935 | 1.49E−10 |
| Nsun2 | 4.46E−23 | 0.25780744 | 0.965 | 0.947 | 8.51E−19 |
| Eif1ax | 6.10E−25 | 0.259171146 | 0.998 | 0.982 | 1.17E−20 |
| Utp11l | 2.11E−21 | 0.247732591 | 0.978 | 0.963 | 4.03E−17 |
| Nifk | 4.74E−16 | 0.25794523 | 0.973 | 0.959 | 9.06E−12 |
| Mrpl36 | 8.39E−15 | 0.203735334 | 0.963 | 0.949 | 1.60E−10 |
| Chchd4 | 3.75E−49 | 0.406592072 | 0.99 | 0.978 | 7.15E−45 |
| Mt1 | 1.69E−19 | 0.330543022 | 0.99 | 0.98 | 3.23E−15 |
| Mcm6 | 5.05E−14 | 0.203330997 | 0.93 | 0.92 | 9.64E−10 |
| 2810004N23Rik | 2.73E−25 | 0.282539829 | 0.982 | 0.973 | 5.21E−21 |
| Lmo4 | 1.74E−66 | 0.775349512 | 0.992 | 0.986 | 3.31E−62 |
| Sms | 1.65E−36 | 0.313663566 | 0.992 | 0.986 | 3.15E−32 |
| Tmem5 | 7.44E−27 | 0.31509393 | 0.949 | 0.943 | 1.42E−22 |
| Abcf1 | 4.64E−25 | 0.277959491 | 0.992 | 0.988 | 8.85E−21 |
| Sfxn1 | 6.98E−21 | 0.212944289 | 0.984 | 0.98 | 1.33E−16 |
| Gm16286 | 8.21E−20 | 0.224472114 | 0.988 | 0.984 | 1.57E−15 |
| Cox7a2l | 1.45E−19 | 0.200215258 | 0.994 | 0.99 | 2.77E−15 |
| Psat1 | 2.81E−16 | 0.206124692 | 0.994 | 0.99 | 5.37E−12 |
| Zfos1 | 5.30E−16 | 0.206256512 | 0.992 | 0.988 | 1.01E−11 |
| Nhp2l1 | 9.94E−34 | 0.239069695 | 1 | 0.998 | 1.90E−29 |
| Txn2 | 8.06E−23 | 0.202261807 | 0.994 | 0.992 | 1.54E−18 |
| Dctpp1 | 1.40E−22 | 0.221067567 | 0.992 | 0.99 | 2.67E−18 |
| Eif3j1 | 8.55E−20 | 0.270419381 | 0.992 | 0.99 | 1.63E−15 |
| Nhp2 | 3.24E−68 | 0.348934627 | 1 | 1 | 6.19E−64 |
| Txnl4a | 6.38E−49 | 0.36485702 | 0.99 | 0.99 | 1.22E−44 |
| Nap1l1 | 1.10E−46 | 0.276547552 | 1 | 1 | 2.10E−42 |
| Srm | 1.22E−45 | 0.356879476 | 0.992 | 0.992 | 2.32E−41 |
| Tomm5 | 1.65E−43 | 0.313429107 | 1 | 1 | 3.15E−39 |
| Dnajc2 | 4.24E−40 | 0.373302174 | 0.988 | 0.988 | 8.10E−36 |
| Ddx21 | 2.72E−35 | 0.383841731 | 0.996 | 0.996 | 5.18E−31 |
| Ncl | 6.24E−31 | 0.351868277 | 1 | 1 | 1.19E−26 |
| Serbp1 | 1.10E−27 | 0.22648657 | 1 | 1 | 2.11E−23 |
| Naa15 | 1.44E−20 | 0.281257486 | 0.982 | 0.982 | 2.75E−16 |
| Map1b | 1.99E−11 | 0.211674236 | 0.949 | 0.949 | 3.79E−07 |
| Gng12 | 3.44E−45 | 0.336166251 | 0.994 | 0.996 | 6.58E−41 |
| Bola2 | 1.95E−33 | 0.243627002 | 0.998 | 1 | 3.72E−29 |
| Ddx18 | 1.13E−20 | 0.236133065 | 0.994 | 0.996 | 2.15E−16 |
| Calm1 | 4.37E−20 | 0.209338392 | 0.998 | 1 | 8.35E−16 |
| Llph | 2.37E−16 | 0.207946587 | 0.994 | 0.996 | 4.52E−12 |
| Hnrnpm | 1.63E−15 | 0.211499543 | 0.99 | 0.992 | 3.11E−11 |
| Nop10 | 2.74E−32 | 0.258763009 | 0.996 | 1 | 5.23E−28 |
| Wdr43 | 1.46E−25 | 0.286052346 | 0.992 | 0.996 | 2.80E−21 |
| mt-Nd3 | 2.70E−23 | 0.241501548 | 0.994 | 0.998 | 5.15E−19 |
| Knop1 | 1.42E−22 | 0.257948217 | 0.992 | 0.996 | 2.71E−18 |
| Dpy30 | 1.40E−15 | 0.206386698 | 0.971 | 0.975 | 2.67E−11 |
| Dph3 | 1.25E−33 | 0.288444631 | 0.982 | 0.988 | 2.38E−29 |
| Anp32b | 6.68E−20 | 0.23155113 | 0.99 | 0.996 | 1.28E−15 |
| Odc1 | 2.58E−14 | 0.212362532 | 0.988 | 0.996 | 4.92E−10 |

iPSCs Emerge Through a Tight Bottleneck from Cells in the MET Region

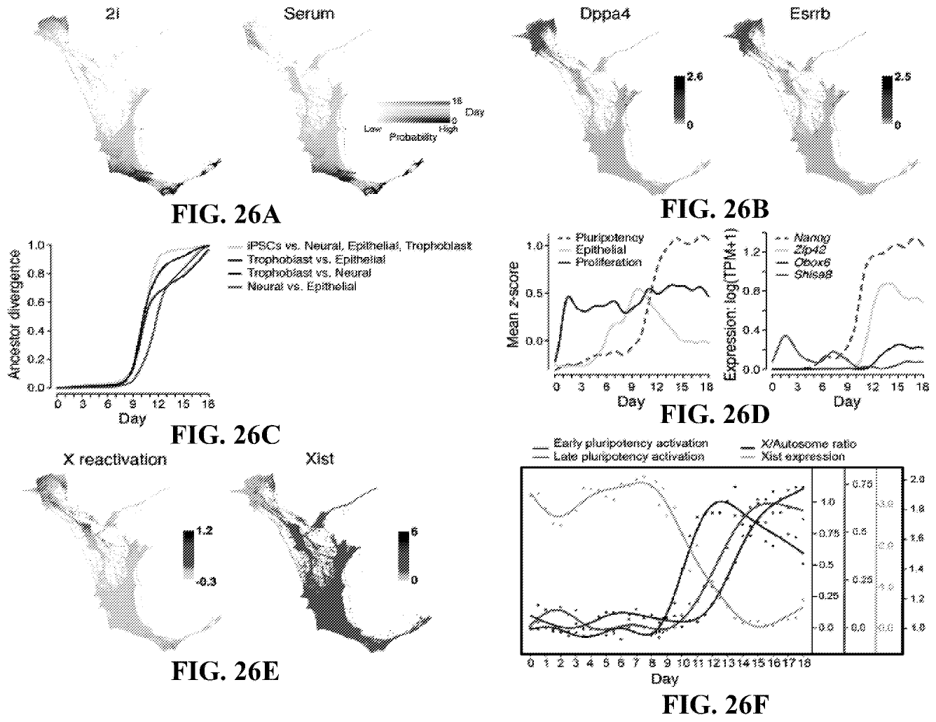

Trajectory analysis showed that cells from the MET region subsequently gained a broad epithelial identity and began to rapidly diverge to give rise the iPS-, epithelial-, trophoblast-, and neural-like cells (FIG. 26A). Importantly, the ancestor distributions of these classes were not distinguishable before the withdrawal of dox at day 8, suggesting that the cells' fates did not appear yet to be determined at that point (FIG. 26B).

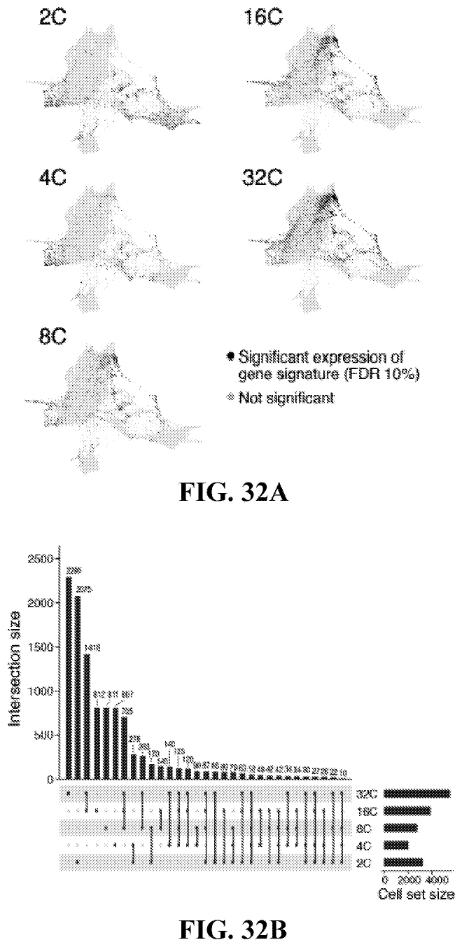

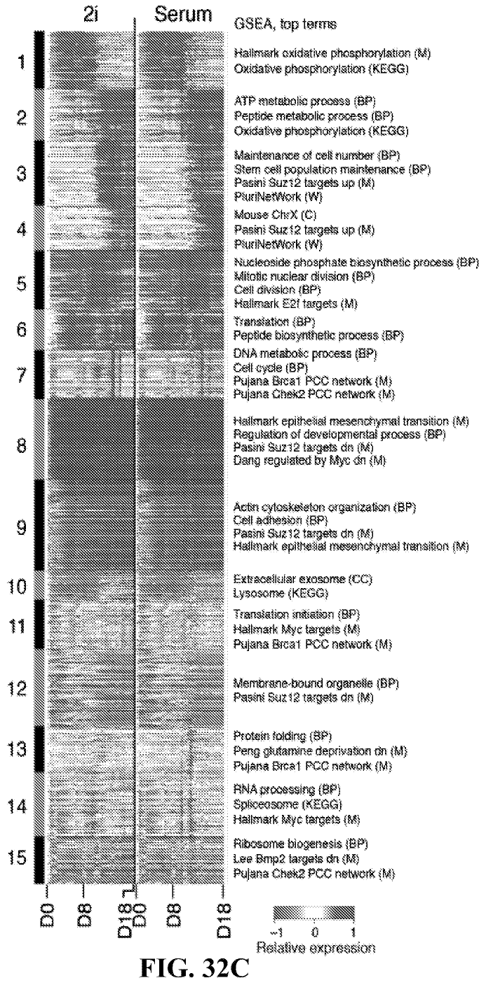

By day 11.5-12.5, the iPS-like cells began to show a clear signature of pluripotency, including canonical marker genes such as Nanog, Sox2, Zfp42, Otx2, Dppa4, and an elevated cell-cycle signature (FIGS. 26C, 26D). In 2i conditions, these iPS-like cells accounted for 12% of cells by day 11.5 and 80-90% from days 15 through 18. In serum conditions, the trend was similar, but the process was delayed by roughly one day and was far less efficient: the pluripotency signature was found in 3.5% of cells by day 12.5 and peaked at just 10-15% from days 15.5 through 18 (FIG. 24G). Notably, we found substantial heterogeneity among the iPSC-related cells. Recent studies reported that a small subset of cells in 2i conditions showed a signature characteristic of the embryonic 2-cell (2C) stage (Falco et al., 2007; Kolodziejczyk et al., 2015; Macfarlan et al., 2012). Scoring our iPS-like cells with signatures based on profiles from 2 cell-, 4 cell-, 8 cell-, 16 cell-, and 32 cell-stage embryos (Goolam et al., 2016) (Table 15, FIG. 32A, 32B), -20% of cells in both 2i and serum conditions showed a 2C, 4C, 8C, 16C, or 32C signature (with roughly half showing signatures for two consecutive stages).

Trajectory analysis suggested that successfully reprogrammed cells passed through a tight bottleneck in days 10-11. The ancestral distribution of iPSCs spanned ˜40% of all cells at day 8.5. It falls to ˜10% of cells at day 10 in 2i conditions and only ˜1% at day 11 in serum conditions. These results suggested that only a small and distinct subset of cells transitioning out of the MET Regions toward various fates had the potential to become iPS cells (below). These iPSC progenitors did not yet fully acquired the pluripotency signature but were changing rapidly toward this fate. They resided along certain thin ‘strings’ in the FLE representation (FIG. 24F, white arrow and 4C, green). iPSC ancestors then rose to ˜40% at day 14 in 2i (and 10% on day 14 in serum), reflecting rapid expansion of pluripotent precursors (FIG. 26C, yellow).

By clustering genes according to similar expression trends along the trajectories to successful reprogramming in 2i and serum conditions, we found induction of various groups of genes involved in regulation of pluripotency, and repression of genes involved in certain metabolic changes and RNA processing (FIG. 32C). Among the upregulated genes, 24 were preferentially expressed in the late stage of reprogramming on successful trajectories and were mostly absent from other cell types; these included Ooep, Fmrlnb, Lncenc1, and Tcl1 (FIG. 32C, Table 17). These genes can be candidate markers for fully reprogrammed cells.

Gene sets related to FIG. 32A

| 1 | 2 | 3 | 4 | 5 | 6 | 7 | 8 |
|---|---|---|---|---|---|---|---|
| Sbspon | Terf1 | Lypla1 | Lactb2 | Pnkd | Rpl7 | Tcea1 | Il1rl1 |
| Dst | 1700007K13Rik | Tceb1 | Igfbp2 | Ptma | Rpl31 | Mcm3 | Fhl2 |
| Nrp2 | Ass1 | Dnpep | Trip12 | Dtymk | H3f3a | Sgol2a | Col3a1 |
| Eef1b2 | Mdk | Tfcp2l1 | Marc2 | Dbi | Rpl7a | Psmd1 | Col5a2 |
| Serpine2 | Chchd5 | Kdm5b | Gm13580 | Snrpe | Rpl12 | R3hdm1 | Sdpr |
| Ephx1 | Praf2 | Swt1 | Hat1 | Cacybp | Zfos1 | Mcm6 | Fn1 |
| Nudt5 | Timm17b | Atp1b1 | Tfpi | Ndufs2 | Pcsk1n | Dhx9 | Col6a3 |
| Commd3 | Hdac6 | Phyh | Platr3 | F11r | Rpl10 | Gm2000 | Gpc1 |
| Ndufa8 | Ndufb11 | Wdr5 | Scand1 | Atp5c1 | Bex2 | Prrc2c | Serpinb2 |
| Ccdc34 | Uxt | Odf2 | Platr27 | Tubb4b | Ndufb5 | Parp1 | Ubxn4 |
| Nop10 | Klhl13 | Rif1 | Fthl17c | Spc25 | Rps3a1 | Nvl | Klhdc8a |
| Knstrn | Slc25a5 | AA467197 | Usp9x | 2700094K13Rik | Apoa1bp | Lbr | Ptgs2 |
| Dtd1 | Ube2a | Slc24a5 | Ndufa1 | Cd59a | Txnip | Enah | Rgs16 |
| Rbck1 | Upf3b | Mrps5 | Gm9 | Eif3m | Gstm1 | Cenpf | Ier5 |
| Nnat | Rhox6 | Eif2s2 | Rhox1 | Rad51 | Rpl34 | Dtl | Soat1 |
| Rbm3 | Rhox9 | Mybl2 | Rhox5 | Spint1 | Rps20 | Yme1l1 | Copa |
| Hmgb3 | Mcts1 | Gtsf1l | Thoc2 | Hypk | Gm11808 | Set | Grem2 |
| Fundc2 | Bcap31 | Wfdc2 | Rbmx2 | Dut | Rps6 | Prrc2b | Col5a1 |
| Slc7a3 | Idh3g | Ncoa3 | Usp26 | 1700037H04Rik | Rps8 | Rpl35 | Angptl2 |
| Hmgn5 | Lage3 | Sall4 | Hprt | Tpx2 | Laptm5 | Hnrnpa3 | Hspa5 |
| 2210013O21Rik | Pbdc1 | Tfap2c | 1700013H16Rik | Ube2c | Rpl11 | Nusap1 | Gorasp2 |
| Rnf13 | Bex4 | Ebp | Fmr1nb | Aurka | Rpl22 | Mga | Creb3l1 |
| Cks1b | Bex1 | Atp6ap2 | Dusp9 | Ppdpf | Rpl9 | Zfp106 | Rcn1 |
| Psmb4 | Wbp5 | Nono | Ssr4 | Plp2 | Rpl5 | Myef2 | Bdnf |
| Bola1 | Ngfrap1 | Alg13 | Dkc1 | Naa10 | Rpl21 | Xrn2 | Thbs1 |
| Gstm5 | Trap1a | Gm8797 | Vbp1 | Pdha1 | Gapdh | Csnk2a1 | Fgf7 |
| Psrc1 | Hsd17b10 | Tpd52 | Pdk3 | Exosc8 | Rps9 | Uba1 | Dstn |
| Cth | Rab9 | Chmp4c | Las1l | Smc4 | Cox6b2 | Gnl3l | Rrbp1 |
| Ndufb6 | Dnajc19 | Lrrc31 | Ogt | Pmf1 | Rpl28 | Huwe1 | Thbd |
| Cdc26 | Lamtor2 | Actl6a | Pin4 | Rab25 | Rps5 | Smc1a | Srxn1 |
| Psip1 | Fdps | Fxr1 | Atrx | Anp32e | Rps19 | Sms | Chmp4b |
| Cdkn2a | Psmd4 | Sox2 | Magt1 | Atp5f1 | Rps16 | Midi | Procr |
| L1td1 | Acp6 | Noct | Cox7b | Stoml2 | Eif3k | 1810022K09Rik | Dlgap4 |
| Tmem59 | Hadh | Platr10 | Pgk1 | Ctnnal1 | Spint2 | Ndufc1 | Ptpn1 |
| Hspb11 | Acer2 | Hiat1 | Rpl36a | Nasp | Cox6b1 | Slc39a1 | Pmepa1 |
| Uqcrh | Slc2a1 | Elovl6 | Prps1 | Cdc20 | Rpl13a | Ilf2 | Slco4a1 |
| Ptprf | Gjb5 | Acadm | Fgd1 | Ppih | Rpl18 | Larp7 | Pgrmc1 |
| Eif3i | Hdac1 | Zfp292 | Prdx4 | Cdca8 | Idh2 | Tet2 | Bgn |
| Atpif1 | Hscb | Aqp3 | A830080D01Rik | Zbtb8os | Rps3 | Fubp1 | Itm2a |
| Stmn1 | Ung | Klf4 | Rbbp7 | Rpa2 | Rpl27a | Anp32b | Fndc3b |
| Eno1 | Cldn4 | Echdc2 | Zrsr2 | Hmgn2 | Rps13 | Smc2 | Sec62 |
| Fgfbp1 | Cldn3 | Gjb3 | Ttc14 | Miip | Rps15a | Zfp462 | Postn |
| Shisa3 | Atp6v1f | Fabp3 | Jade1 | Apitd1 | Uqcrc2 | Pum1 | Fam198b |
| Scarb2 | Mkrn1 | Rps6ka1 | Vangl1 | Park7 | Ypel3 | Srrm1 | S100a7a |
| Cops4 | Cct7 | Rsrp1 | Ak4 | Tyms | Ifitm3 | Rcc2 | Crct1 |
| Gltp | Nfu1 | Tcea3 | Fblim1 | Cenpa | Rplp2 | Gm26825 | Ngf |
| Pop5 | Slc2a3 | Usp48 | Zfp600 | Qdpr | Mrpl23 | Tomm7 | Rhoc |
| Pebp1 | Fkbp4 | Alpl | Gm13251 | Med28 | Rps12 | 4930548H24Rik | Csf1 |
| Rpl6 | Ldhb | Gm13154 | 2610305D13Rik | Paics | Rps15 | Rfc1 | Col11a1 |
| Ran | AU018091 | Agtrap | Fbxo6 | G3bp2 | Rpl6l | Grsf1 | F3 |
| Mospd3 | Lig1 | Insig1 | Rbpj | Hnrnpdl | Naca | Hnrnpd | Ostc |
| Hmgb1 | Bcam | Dnajb6 | Crlf2 | Cit | Rps26 | Golga3 | Cyr61 |
| Ndufa4 | Exosc5 | Yes1 | Ppp1cc | Rfc5 | Ndufa13 | Mcm7 | Bcl10 |
| Podxl | Gmfg | Lap3 | Arf5 | Chchd2 | Rpl18a | Luc7l2 | Glipr2 |
| Akr1b3 | Map4k1 | Kit | Stra8 | Rfc2 | Bst2 | Cbx3 | Sec61b |
| Hnrnpa2b1 | Ppp1r14a | Rest | Ube2s | Atp5j2 | Cox4i1 | Immt | Tnc |
| Lsm3 | Tbcb | Spp1 | Zfp787 | Lsm5 | Rpl13 | Tmsb10 | Eva1b |
| Trh | Gpi1 | Mtf2 | Tmem160 | Tcf7l1 | Rpl15 | Dqx1 | Errfi1 |
| Mgst1 | Etfb | Pxmp2 | Calm3 | Suclg1 | Rps24 | Mcm2 | Ost4 |
| Trappc6a | Ucp2 | Ulk1 | Zfp428 | Tpi1 | Rpl23a-ps3 | Ptms | Ugdh |
| Dmrtc2 | Folr1 | Med13l | Plekha4 | Cdca3 | Rpl13-ps3 | Aebp2 | Apbb2 |
| Fbl | Mrpl17 | Tbx3 | Arrdc4 | Lockd | Rps25 | Fam60a | Igfbp7 |
| Krtdap | Arl6ip1 | Sbno1 | Eif3f | Peg3 | Fxyd6 | Trim28 | Cxcl5 |
| Prmt1 | Aldoa | Cops6 | Sept1 | Gltscr2 | Rpl10-ps3 | Hnrnpl | Ppbp |
| Bax | Pycard | Slc25a13 | Ctbp2 | Sae1 | Rpl4 | Polr2i | Cxcl3 |
| Ldha | Bnip3 | Asns | Sycp3 | Lsr | Gsta4 | Sema4b | Cxcl1 |
| Tm2d3 | Utf1 | Trim24 | Nudt4 | Ruvbl2 | Eef1a1 | Prc1 | Cxcl2 |
| l7Rn6 | Ifitm2 | Zc3hav1 | Sap30 | Bcat2 | Rpl29 | Blm | Ereg |
| Ndufc2 | Cenpw | Ezh2 | Gm2694 | Snrpn | Rpsa | RP23-4H17.3 | U90926 |
| Ndufab1 | Ddit4 | Tra2a | Fam25c | Coq7 | Rpl14 | Bclaf1 | Rsrc2 |
| Tmem219 | Cisd1 | Gdf3 | Sap18 | Plk1 | Rps27a | Ptges3 | Denr |
| Vkorc1 | Ddt | Dppa3 | Klf5 | Spns1 | Gnb2l1 | Arglu1 | Ubc |
| Mki67 | Chchd10 | Nanog | Khdc3 | Dctpp1 | Rpl26 | Mcm5 | Serpine1 |
| Glrx3 | Pfkl | Lpcat3 | Ooep | Fbxo5 | Rpl23 | Smarca5 | Pcolce |
| Cd81 | Polr2e | Cd9 | Higd1a | Sf3b5 | Rpl19 | Cnot1 | Kdelr2 |
| Perp | Gpx4 | 2810474O19Rik | Mrps24 | Cdk1 | Rpl27 | Rps26-ps1 | Cav1 |
| Mif | Cirbp | Apoc1 | Eif4a1 | Lsm7 | Dcxr | Aars | Flnc |
| Atp5d | 1500009L16Rik | Apoe | C1qbp | Eef2 | Rps23 | Ankrd11 | Ptn |
| Ndufs7 | Prim1 | Pvrl2 | Suz12 | Mrpl42 | Btf3 | Wapl | Capg |
| Uqcr11 | Eif4ebp1 | Cox7a1 | AI662270 | Cct2 | Rps7 | Rpgrip1 | Rab7 |
| Oaz1 | Ankrd37 | Tdrd12 | Dynll2 | Atp5b | Wdr89 | Supt16 | Fbln2 |
| Slc25a3 | Cope | Tead2 | E130012A19Rik | Ormdl2 | Rpl30 | Zc3h13 | Sec13 |
| Ndufa12 | Sin3b | Gtf2h1 | Gna13 | Sarnp | Gm10020 | Uchl3 | Cxcl12 |
| Cnpy2 | Syce2 | Spty2d1 | Snhg20 | Hmgb2 | Rpl8 | Anapc13 | Tspan9 |
| Nabp2 | Asna1 | Mfge8 | Tex19.1 | Lsm4 | Rpl3 | Gnai2 | Arhgdib |
| Slc25a4 | Mt1 | Ticrr | Pfkp | Tecr | Rpl35a | Uqcr10 | Il11 |
| Apela | 2700060E02Rik | Zfand6 | Tubb2b | Orc6 | Gm9843 | Actr2 | Ehd2 |
| Isyna1 | Mrps16 | Eed | H2afy | Nudt21 | Sod1 | Canx | Pvr |
| Mrpl34 | Tkt | Tmem41b | Cox7c | Cdh1 | Psmb1 | Alkbh5 | Plaur |
| Ndufb7 | Mphosph8 | Gga2 | Lncenc1 | Psmb5 | Ndufb10 | Ncor1 | Psmd8 |
| Prdx2 | Esco2 | Nfatc2ip | Nampt | Dhrs4 | Rps10 | Pfas | Fxyd5 |
| Pllp | Bnip3l | Mylpf | Ifi27 | Cdca2 | Rpl10a | Naa38 | Rcn3 |
| Got2 | Sugt1 | Echs1 | Tcl1 | Spc24 | Ddah2 | Xaf1 | Klf13 |
| Psmb10 | Pigyl | Ifitm1 | Papola | H2afx | Gm26917 | Ywhae | Vimp |
| Rab4a | Psma4 | Taldo1 | Apobec3 | Slc35f2 | AY036118 | Taf15 | Lrrc32 |
| Dnajc9 | Cox5a | Fgf4 | Smc1b | Pkm | 2410015M20Rik | Npepps | Map6 |
| Itm2b | Morf4l1 | Akap12 | Pim3 | Anp32a | Rpl27-ps3 | Top2a | Adm |
| Atp5l | H2afv | Sgk1 | Rpl39l | Snapc5 | Gm10036 | Acly | Mical2 |
| Cadm1 | Commd1 | Tet1 | Eif4a2 | Tipin | Prelid2 | Bptf | Tgfb1i1 |
| Crabp1 | Pttg1 | Spic | Adprh | Ccnb2 | Rps14 | Fasn | Rnh1 |
| 2810417H13Rik | Psmb6 | Csrp2 | Dppa4 | Cox7a2 | Rpl17 | Slc16a3 | H19 |
| Rps27l | Psmd12 | Baz2a | Dppa2 | Gpx1 | Gm6133 | Dek | Igf2 |
| Gtf2a2 | Atp5h | Ash2l | Cggbp1 | Impdh2 | Fau | Rbm25 | Cttn |
| Hmgn3 | Galk1 | Zfp42 | Morc3 | Ndufaf3 | Cox8a | Dnajc21 | Rgs17 |
| Nf2 | Psma2 | Tmem192 | Brwd1 | Uqcrc1 | Eef1g | Myo10 | Ctgf |
| Ramp3 | Acot13 | Nr2c2ap | Tmem181a | Zmat5 | Gm9493 | Rad21 | Sar1a |
| Mdh1 | Uqcrb | Klf2 | Dynlt1a | Pold2 | Rpl9-ps6 | St13 | Col6a2 |
| Hint1 | Cetn3 | Anapc10 | Mpc1 | Snrnp25 | Gsto1 | Lima1 | Pofut2 |
| Aldh3a1 | Dhfr | Dnase2a | Pgp | Npm1 | Rps12-ps3 | Usp7 | Pttg1ip |
| Poldip2 | Mycn | Mt2 | Gfer | Hmmr | mt-Co2 | Etv5 | Bsg |
| Krt19 | Psma6 | Gabarapl2 | Pim1 | Cdkn2aipnl | | Tfrc | Timp3 |
| Krt17 | Fkbp3 | Kat6b | Myo1f | Tmem107 | | Gsk3b | Btg1 |
| Itgb4 | Atp6v1d | Hesx1 | Dhx16 | Cldn7 | | Cox17 | Atp2b1 |
| Sec14l1 | Brix1 | Zfhx2 | Dazl | Atp5g1 | | Gm8186 | Rap1b |
| Tk1 | Cox6c | Rnaseh2b | Vapa | Cbx1 | | Srpk1 | Ndufa4l2 |
| Stard3nl | Eif3e | Tdh | Ralbp1 | Psmb3 | | Stk38 | Myl6 |
| Hist1h1b | Tonsl | Rgcc | Arl14epl | Jup | | Brd4 | Hmox1 |
| Hist1h1e | Gcat | Zbtb44 | Prrc1 | Dcakd | | Gm42418 | Junb |
| Uqcrfs1 | Syngr1 | Rpp25 | Fbxo15 | Sumo2 | | Uhrf1 | Mmp2 |
| Eci2 | Cenpm | Rbpms2 | Gstp2 | Birc5 | | Khsrp | Gm22 |
| Ndufs6 | Ndufa6 | U2surp | D030056L22Rik | Stra13 | | Birc6 | Acta1 |
| Mrps36 | Atp5g2 | Slc25a36 | | Hist1h2ae | | Erdr1 | Nrp1 |
| Id2 | Pam16 | Amt | | Gmnn | | Matr3 | Vcl |
| Rtn1 | Pigx | Arih2 | | Cks2 | | Stip1 | Arf4 |
| Siva1 | Ndufb4 | Slc25a20 | | Higd2a | | Incenp | Selk |
| Ahnak2 | Dynlt1f | Tdgf1 | | Ccnb1 | | Tmem258 | Mustn1 |
| Nudt14 | Thoc6 | Trim71 | | Rrm2 | | Hells | Spcs1 |
| Crip2 | Tceb2 | Upp1 | | Mis18bp1 | | Scd2 | Fermt2 |
| Ptp4a3 | Ccnf | Cct4 | | Mthfd1 | | Eif3a | Gjb2 |
| Ly6a | Ndufv3 | Skp1a | | Cct5 | | mt-Nd1 | Ubl5 |
| Eef1d | Ndufa7 | Vdac1 | | Cyc1 | | | Col5a3 |
| Tst | Tubb5 | Gm2a | | Eif3l | | | Cnn1 |
| H1f0 | Rpp21 | Mpdu1 | | Tuba1b | | | Oaf |
| Pmm1 | Znrd1 | Tmem256 | | Krt8 | | | Thy1 |
| Samm50 | Oard1 | Scpep1 | | Hnrnpa1 | | | Trappc4 |
| Eif4b | Ndufv2 | Igf2bp1 | | Mrpl40 | | | Ncam1 |
| 2610318N02Rik | Tgif1 | Calcoco2 | | Rfc4 | | | Wdr61 |
| Dgcr6 | Cebpzos | Dnajc7 | | Bbx | | | Cspg4 |
| Fetub | Mta3 | Slc25a39 | | Ezr | | | Sema7a |
| Atp5o | Pfdn1 | Grn | | Acat2 | | | Loxl1 |
| Agpat4 | Impa2 | Ccdc43 | | Cldn6 | | | Mapk6 |
| Nme4 | Smc3 | Ttyh2 | | Ppil1 | | | Col12a1 |
| Mapk13 | | Wbp2 | | U2af1 | | | Amotl2 |
| Cd320 | | Ubald2 | | Pfdn6 | | | Selm |
| Ly6g6c | | Jarid2 | | Lsm2 | | | Xbp1 |
| Ly6g6f | | Ubxn2a | | Polr1c | | | Aebp1 |
| Dnph1 | | 1110008L16Rik | | Ndufa11 | | | Ykt6 |
| Cox7a2l | | Esrrb | | Crb3 | | | Tns3 |
| Pigf | | Ckb | | Myl12b | | | Sec61g |
| Ecscr | | Atxn10 | | Dpy30 | | | Sertad2 |
| Cyb5a | | Slc25a1 | | Epcam | | | Rtn4 |
| Rnaseh2c | | Morc1 | | Paip2 | | | Adam19 |
| Trmt112 | | Jam2 | | Lmnb1 | | | Sqstm1 |
| Carnmt1 | | Wtap | | Atp5a1 | | | Sparc |
| Avpi1 | | Sod2 | | Ndufs8 | | | Kctd11 |
| Ndufb8 | | Rnf5 | | Rbm4b | | | GabaraP |
| Cuedc2 | | Zfp57 | | Banf1 | | | Cxcl16 |
| Sfr1 | | Cdc5l | | Mrpl49 | | | Tax1bp3 |
| | | Slc29a1 | | Arl2 | | | Pafah1b1 |
| | | Gm7325 | | Fkbp2 | | | Serpinf1 |
| | | Ccnd3 | | | | | Ift20 |
| | | Ppm1b | | | | | Ccl2 |
| | | Msh2 | | | | | Ccl5 |
| | | Msh6 | | | | | Vmp1 |
| | | Cystm1 | | | | | Col1a1 |
| | | Taf7 | | | | | Copz2 |
| | | Dcp2 | | | | | Igfbp4 |
| | | Snx2 | | | | | Eif1 |
| | | Cndp2 | | | | | Timp2 |
| | | Chka | | | | | Klf6 |
| | | Ubxn1 | | | | | Inhba |
| | | Klf9 | | | | | Serpinb6a |
| | | Scd1 | | | | | Card19 |
| | | mt-Co1 | | | | | Pdlim7 |
| | | | | | | | Tmed9 |
| | | | | | | | Smim15 |
| | | | | | | | Plk2 |
| | | | | | | | Rhob |
| | | | | | | | Nfkbia |
| | | | | | | | Arf6 |
| | | | | | | | Frmd6 |
| | | | | | | | Actn1 |
| | | | | | | | Ltbp2 |
| | | | | | | | Dlk1 |
| | | | | | | | Tnfaip2 |
| | | | | | | | Crip1 |
| | | | | | | | Snhg18 |
| | | | | | | | Cthrc1 |
| | | | | | | | Ext1 |
| | | | | | | | Has2 |
| | | | | | | | Wisp1 |
| | | | | | | | Myh9 |
| | | | | | | | Lgals1 |
| | | | | | | | Kdelr3 |
| | | | | | | | Atf4 |
| | | | | | | | Tuba1c |
| | | | | | | | Itga5 |
| | | | | | | | Vasn |
| | | | | | | | Col8a1 |
| | | | | | | | Ier3 |
| | | | | | | | Ppp1r11 |
| | | | | | | | Vegfa |
| | | | | | | | Ltbp1 |
| | | | | | | | Crim1 |
| | | | | | | | Fez2 |
| | | | | | | | Cdc42ep3 |
| | | | | | | | Zfp36l2 |
| | | | | | | | Hbegf |
| | | | | | | | Yipf5 |
| | | | | | | | Lox |
| | | | | | | | Ier3ip1 |
| | | | | | | | Efemp2 |
| | | | | | | | Ehbp1l1 |
| | | | | | | | Ehd1 |
| | | | | | | | Fads3 |
| | | | | | | | Ankrd1 |
| | | | | | | | Dusp5 |

| 9 | 10 | 11 | 12 | 13 | 14 | 15 |
|---|---|---|---|---|---|---|
| Map4k4 | Snhg6 | Ptp4a1 | Bag2 | Sdhaf4 | Imp4 | Eif5b |
| Bzw1 | Mpzl1 | Actr1b | Mrpl30 | Sumo1 | Tuba4a | Nop58 |
| Raph1 | Creg1 | Hspd1 | Hspe1 | Aamp | Ncl | Rpl37a |
| Arpc2 | Uap1l1 | Bok | Acadl | Eif4e2 | Ssna1 | Myeov2 |
| Tmbim1 | Ptges | Tsn | Stk16 | Timm17a | Surf2 | Sept2 |
| Lrrfip1 | Serf2 | Nucks1 | Adipor1 | Ufc1 | Urm1 | Ddx18 |
| Ube2f | Slc20a1 | Tpr | Phlda3 | Pfdn2 | Ppp2r4 | B930036N10Rik |
| Hdlbp | Cst3 | Uck2 | Prdx6 | Hspa14 | Dpm2 | Nmt2 |
| Nifk | Gss | Hnrnpu | Mpc2 | Edf1 | Arpc5l | Sptan1 |
| Actr3 | Sdc4 | Eprs | Mgst3 | Dnlz | Timm10 | Exosc2 |
| Csrp1 | Adrm1 | Smyd2 | Cnih4 | 1110008P14Rik | Ssrp1 | Dync1i2 |
| Arpc5 | Lamp2 | Rbm17 | Aida | Tor2a | Snrpb | Psmc3 |
| Qsox1 | Renbp | Agpat2 | St6galnac4 | Psmb7 | Ppid | Usp50 |
| Prrx1 | S100a1 | Fbxw2 | Pdia3 | Dnmt3b | Gpatch4 | Cse1l |
| Tmco1 | S100a13 | Mtx2 | Mrps26 | Tgif2 | Jtb | Atp5e |
| Tagln2 | Cnn3 | Caprin1 | Naa20 | Rpn2 | Nras | Rps21 |
| Wdr26 | Atp6v1g1 | 1500011K16Rik | Fkbp1a | Tceal8 | Gar1 | Plk4 |
| Degs1 | Tm2d1 | Nop56 | Id1 | Morf4l2 | Cenpe | Naa15 |
| Capn2 | Atp6v0b | Snx5 | Dynlrb1 | Fabp5 | Sep15 | Rps27 |
| Rrp15 | Snhg12 | Raly | Romo1 | Car2 | Ebna1bp2 | Mrpl9 |
| Hacd1 | Sh3bgrl3 | 1110008F13Rik | Samhd1 | Selt | Svbp | Sars |
| Surf4 | Pdpn | Srsf6 | Top1 | Cct3 | Mrps15 | Agl |
| Ptrh1 | Smim14 | Sys1 | Pfdn4 | Ssr2 | Thrap3 | Ccne2 |
| Fam129b | Cox18 | Rae1 | Gnas | Rbm8a | Ak2 | Otud6b |
| Gsn | Hspb8 | Ddx3x | Ctsz | 1810037I17Rik | Tmem234 | Vcp |
| Rbms1 | Tmem120a | Vma21 | Slmo2 | Ube2d3 | Zcchc17 | Tex10 |
| Grb14 | Arpc1b | Ccna2 | Fhl1 | Dnaja1 | Hnrnpr | Tmem245 |
| Zak | Gpnmb | Tpm3 | G6pdx | Clta | Ddost | Lepr |
| Nfe2l2 | Malsu1 | Atp1a1 | Xist | Prdx1 | Mrto4 | Ccdc163 |
| Nckap1 | Pole4 | Csde1 | Sh3bgrl | Psmb2 | Sdhb | Ybx1 |
| Zc3h15 | Chmp2a | Eif4e | Tmem35 | Marcksl1 | Szrd1 | Gm13075 |
| Itgav | Vasp | Ddah1 | Ammecr1 | Trnau1ap | Mrpl20 | Noc2l |
| Cd44 | Rabac1 | Rad23b | Eif1ax | Nudc | Aurkaip1 | Fam133b |
| Emc7 | Blvrb | Ndc1 | Stmn2 | Sfn | Lrpap1 | Abcb1b |
| Eif3j1 | Capns1 | Ctps | Lhfp | Tmem60 | Mrfap1 | Dhx15 |
| B2m | Dkkl1 | Pabpc4 | Tm4sf1 | Ppp1cb | Lyar | Noa1 |
| Fbn1 | Nupr1 | Mycbp | Mbnl1 | Slbp | Dynll1 | Atp5k |
| Prnp | Snx3 | Sfpq | Lxn | Plac8 | Cox6a1 | Pdap1 |
| H13 | Psap | Ptp4a2 | Hdgf | Anapc5 | Arl6ip4 | Ndufa5 |
| Pdrg1 | Cstb | Ythdf2 | Mex3a | Por | Mrps17 | Rbm28 |
| Mapre1 | Gadd45b | Srm | S100a16 | Ywhag | Eif4h | Pdia4 |
| Eif6 | Arl1 | Gnb1 | S100a10 | Capza2 | Mdh2 | Serbp1 |
| Myl9 | Ddit3 | Nadk | Mrps21 | Gstk1 | Fis1 | Hk2 |
| Ywhab | Cd63 | Dbf4 | Phgdh | Ruvbl1 | Znhit1 | Paip2b |
| Timp1 | Ifi30 | Dnajc2 | Camk2d | Arpc4 | Fscn1 | Snrpg |
| Hs6st2 | Hsbp1 | Abcf2 | Cisd2 | Hnrnpf | Arpc1a | Gmcl1 |
| Flna | Map1lc3b | Rheb | Fam92a | M6pr | Pomp | Wbp11 |
| Msn | Cyba | Ppm1g | Tmem55a | Mlf2 | 2610001J05Rik | Dennd5b |
| Sat1 | Tomm20 | Iscu | Ggh | Cops7a | Cycs | Ndufa3 |
| Sh3kbp1 | Ghitm | Mlec | Tomm5 | Golt1b | Vamp8 | Cnot3 |
| Anxa5 | Psme2 | Rnf10 | Txn1 | Clptm1 | Fam136a | U2af2 |
| Ufm1 | Ctsb | Atp2a2 | Nfib | Psmc4 | Cnbp | Iqgap1 |
| Dclk1 | Srpr | Gnb2 | Scp2 | Nup62 | Hmces | Ipo7 |
| Wwtr1 | Tbrg1 | Eif3b | Kti12 | Mesdc2 | Chchd4 | Tead1 |
| Serp1 | Hexa | Fam220a | Akr1a1 | Ppp4c | Emg1 | 1110004F10Rik |
| Ssr3 | Rab11a | Ccz1 | Macf1 | Bccip | Phb2 | Knop1 |
| Crabp2 | Spg21 | Bri3 | Utp11l | Phlda2 | Mrpl51 | Bola2 |
| Lmna | Ppib | Gtf3a | Wasf2 | Ltv1 | Tsen34 | Fus |
| S100a4 | Rhoa | Hsph1 | Mtfr1l | Zwint | Napa | Hras |
| S100a11 | Pdlim4 | Mat2a | Id3 | Ube2n | Mrps12 | Polr2l |
| Vcam1 | Cd68 | Mthfd2 | Hspg2 | Myl6b | Nudt19 | Ap2a2 |
| Snx7 | Ggnbp2 | H2afj | Minos1 | Fam32a | Emc10 | Amd1 |
| Ppp3ca | Nid1 | Strap | Acot7 | Ddx39 | Grwd1 | Ddx21 |
| Pdlim5 | Ninj1 | Bcat1 | Atad3a | Ier2 | Snrpa1 | Cdc34 |
| Lmo4 | Ctsl | Slc1a5 | Cdk6 | Calr | Mrps11 | Metap2 |
| Sh3glb1 | Gm10116 | Tomm40 | Sri | Cnep1r1 | Aen | Pet100 |
| Gng5 | Glrx | Eif4g2 | Mrpl33 | Mt4 | Clns1a | Timm44 |
| Wls | Twistnb | Gde1 | Grpel1 | Ciapin1 | Tufm | Haus8 |
| Chchd7 | Npc2 | Mettl9 | Limch1 | Gcsh | Ino80e | Gfod2 |
| Impad1 | Dap | Eif3c | Ociad1 | Emc8 | Bckdk | Nip7 |
| Rab2a | Ndrg1 | Kcnq1ot1 | Ociad2 | Chmp1a | Bub3 | 2810004N23Rik |
| Ndufaf4 | Cyb5r3 | Rwdd1 | Sept11 | Gnpnat1 | Urah | Gnl3 |
| Ube2j1 | Tmbim6 | Ppa1 | Anxa3 | Bmp4 | Nap1l4 | Nisch |
| Tpm2 | Litaf | Mbd3 | Pdgfa | Dad1 | Snrpd3 | Ktn1 |
| Tln1 | Hacd2 | Abhd17a | Rac1 | Tsc22d1 | Sumo3 | Mrpl52 |
| Plin2 | Hcfc1r1 | Map2k2 | Kpna7 | Aasdhppt | Timm13 | Loxl2 |
| Mtap | Atp6v0e | Aes | Polr1d | Rpusd4 | Thop1 | Gm10076 |
| Jun | Ostf1 | Rtcb | Shfm1 | Oaz2 | Dohh | Taf1d |
| Jak1 | Pdlim1 | Nap1l1 | Lsm8 | Fam96a | Yeats4 | Gm26737 |
| Mast2 | | Cs | 1810058I24Rik | Rsl24d1 | Cdk4 | Arpp19 |
| Elovl1 | | Dlc1 | Gng12 | Rnf7 | Pa2g4 | Rps27rt |
| Txlna | | Abce1 | Aup1 | Rbp1 | Lsm1 | Limk2 |
| Clic4 | | Dnaja2 | Bola3 | Rrp9 | Fkbp8 | Nudcd3 |
| Cdc42 | | E2f4 | Actg2 | Nme6 | Ccdc124 | Hnrnpab |
| Nppb | | Psmd7 | Arl6ip5 | Ewsr1 | Dda1 | Larp1 |
| Pgd | | Dcun1d5 | Foxp1 | Arf1 | Rbmxl1 | Mybbp1a |
| Cgref1 | | Rp9 | Rhno1 | Trp53 | Lsm6 | Ap2b1 |
| Ywhah | | Ei24 | Magohb | Car4 | D8Ertd738e | Cite |
| Gm1673 | | Rdx | Ybx3 | Slc35b1 | 2310036O22Rik | Nfe2l1 |
| Wdr1 | | Imp3 | Epn1 | H3f3b | Cmc2 | Pcgf2 |
| Pcdh7 | | Polr2m | Sepw1 | Gaa | Aprt | Nmt1 |
| Tpst2 | | Cdv3 | Gemin7 | Anapc11 | Vdac2 | Ddx5 |
| Coro1c | | Map4 | Egln2 | Dus1l | Apex1 | Rpl38 |
| Tmed2 | | G3bp1 | Tmem147 | Pak1ip1 | Nedd8 | Srsf2 |
| Ap1s1 | | Srsf1 | Pdcd5 | Emb | N6amt2 | Prpf4b |
| Fam20c | | Lrrc59 | Josd2 | Pdia6 | Reep4 | Hnrnpa0 |
| Actb | | Snf8 | Akt1s1 | Ywhaq | Pin1 | Nsa2 |
| Cyth3 | | Kpnb1 | Igf1r | Max | Tmed1 | Smn1 |
| Slc7a1 | | Psme3 | Serpinh1 | Eif2s1 | Ecsit | Rps29 |
| Col1a2 | | Lsm12 | Rrm1 | Srsf5 | Elof1 | Slirp |
| Tes | | Fam104a | Prkcdbp | Ahsa1 | Hmbs | 2010107E04Rik |
| Calu | | Prpsap1 | Parva | Sub1 | Manf | Rpl37 |
| Cald1 | | Gps1 | Tspan4 | Mcrs1 | Tma7 | Wdr70 |
| Mtpn | | Gdi2 | Ccnd1 | Tarbp2 | Ccdc12 | Polr2k |
| Zyx | | Rala | Epb41l2 | Copz1 | C1d | Rangap1 |
| Tex261 | | Ssr1 | Mareks | Glyr1 | Nhp2 | Hes1 |
| Cyp26b1 | | B230219D22Rik | Cd24a | Ube2v2 | Uqcrq | Son |
| Sec61a1 | | Cxcl14 | Gja1 | Ap2m1 | Atox1 | Snhg9 |
| Brk1 | | Hnrnpk | Arid5b | Dnajb11 | Guk1 | Hnrnpm |
| Ltbr | | Nsun2 | Plpp2 | Cct8 | Rangrf | Rps28 |
| Gabarapl1 | | Rab10 | Snrpf | Tcp1 | Eif5a | Abcf1 |
| Emp1 | | Smc6 | Atxn7l3b | Rab11b | Tmem97 | Ptcra |
| Ercc1 | | Odc1 | Shmt2 | Mrps18b | Nme1 | Sgol1 |
| Cd3eap | | Srp54b | Lrp1 | Mea1 | Mrpl27 | Wdr43 |
| Axl | | Glrx5 | Col4a2 | Calm2 | Phb | Cebpz |
| Actn4 | | Eif5 | Ckap2 | Polr2d | Coa3 | Epb41l4aos |
| 2200002D01Rik | | Pabpc1 | Vps36 | Eif1a | Ict1 | Ndufa2 |
| Atf5 | | Ly6e | Fgfr1 | BC031181 | Hn1 | Rbm22 |
| Emp3 | | Pcbp2 | Nrg1 | Pgam1 | Mrps7 | Tcof1 |
| Prss23 | | Rsl1d1 | Uba52 | Xpnpep1 | 1810043H04Rik | Nars |
| Rrp8 | | Gspt1 | Pgls | mt-Co3 | Mrpl12 | Ddb1 |
| Ilk | | Mapk1 | Scoc | mt-Nd4 | Tmem14c | Nmrk1 |
| Rras2 | | Eif4g1 | Nfix | | Nop16 | Usmg5 |
| Pik3c2a | | Ppp1r2 | Arl2bp | | Prelid1 | Pdcd11 |
| Itpripl2 | | 0610012G03Rik | Gm10073 | | Lman2 | mt-Nd2 |
| Tnrc6a | | Naa50 | Zfhx3 | | Ddx46 | mt-Atp8 |
| Cdipt | | Tomm70a | 2310022B05Rik | | 2010111I01Rik | mt-Nd3 |
| Abracl | | Srrm2 | Ube2e1 | | Mrpl36 | mt-Nd4l |
| Col6a1 | | Kif5b | Dph3 | | Sf3b6 | mt-Nd5 |
| Slc19a1 | | Etf1 | Anxa8 | | Sptssa | |
| Ube2g2 | | Hspa9 | Cnih1 | | Erh | |
| Cnn2 | | Ube2d2a | Lgals3 | | Tmed10 | |
| Nfic | | Psat1 | Tpt1 | | Snw1 | |
| Ncln | | Npm3 | Mbnl2 | | Zfp706 | |
| Txnrd1 | | | Smco4 | | 9130401M01Rik | |
| Ckap4 | | | Rexo2 | | Chrac1 | |
| Elk3 | | | Cryab | | Polr2f | |
| Phlda1 | | | Anxa2 | | Tomm22 | |
| Llph | | | Nedd4 | | Adsl | |
| Hmga2 | | | Cd109 | | Rbx1 | |
| Tmem5 | | | Irak1bp1 | | Phf5a | |
| Col4a1 | | | Syncrip | | Nhp2l1 | |
| Tm2d2 | | | Pcolce2 | | Rrp7a | |
| Rwdd4a | | | Mras | | Tuba1a | |
| Cpe | | | Pcbp4 | | Ranbp1 | |
| Tpm4 | | | Ifrd2 | | Hmgn1 | |
| Dnajb1 | | | Cmtm7 | | Tmem242 | |
| Piezo1 | | | Purb | | Mrpl18 | |
| Tcf25 | | | Grb10 | | Rnps1 | |
| Itgb1 | | | Sptbn1 | | Ube2i | |
| Flnb | | | Ccng1 | | Stub1 | |
| Gch1 | | | Chd3 | | Mrpl28 | |
| Pnp | | | Pfn1 | | Srsf3 | |
| Mmp14 | | | Txndc17 | | Glo1 | |
| Esd | | | Emc6 | | Mrpl14 | |
| Kctd12 | | | Nxn | | Srsf7 | |
| Dnajc3 | | | Timm22 | | Snrpd1 | |
| Ipo5 | | | Ccl7 | | Hdac3 | |
| Amotl1 | | | Dusp14 | | Cdk2ap2 | |
| Tagln | | | Nme2 | | Coro1b | |
| Pafah1b2 | | | Spop | | Ppp1ca | |
| Rcn2 | | | Fkbp10 | | Mrpl11 | |
| Csk | | | Ptrf | | Sf3b2 | |
| Tpm1 | | | Becn1 | | Eif1ad | |
| Bnip2 | | | Vat1 | | Cfl1 | |
| Tmed3 | | | Limd2 | | Sssca1 | |
| Plscr1 | | | Syngr2 | | Polr2g | |
| Rassf1 | | | Fam195b | | Tmem109 | |
| Prkar2a | | | Hist1h2ap | | Prpf19 | |
| Crtap | | | Fam120a | | Rcl1 | |
| Slc35e4 | | | Gadd45g | | Nolc1 | |
| Ccm2 | | | Sfxn1 | | Zdhhc6 | |
| Anxa6 | | | Cltb | | mt-Cytb | |
| Mprip | | | Serf1 | | | |
| Map2k3 | | | Mast4 | | | |
| Pitpna | | | Sdc1 | | | |
| Myo1c | | | Sox11 | | | |
| Fam101b | | | Bzw2 | | | |
| Tnfaip1 | | | Baz1a | | | |
| Mmd | | | Fam177a | | | |
| Ccdc137 | | | Timm9 | | | |
| P4hb | | | Synj2bp | | | |
| Arhgdia | | | Calm1 | | | |
| Sox4 | | | Meg3 | | | |
| Tubb2a | | | Akt1 | | | |
| Pxdc1 | | | Oxct1 | | | |
| Txndc5 | | | Ywhaz | | | |
| Bicd2 | | | Eny2 | | | |
| Tgfbi | | | Myc | | | |
| Pdcd6 | | | Txn2 | | | |
| Vcan | | | Polr3h | | | |
| Tmem167 | | | Zcrb1 | | | |
| Zcchc9 | | | Dazap2 | | | |
| Map1b | | | Prr13 | | | |
| Gpx8 | | | Carhsp1 | | | |
| Fst | | | Emp2 | | | |
| Rock2 | | | Fam162a | | | |
| Fam110c | | | Fstl1 | | | |
| Ifrd1 | | | Chmp2b | | | |
| Cfl2 | | | Cdkn1a | | | |
| Mgat2 | | | Clic1 | | | |
| Flrt2 | | | Mydgf | | | |
| Fbln5 | | | Memo1 | | | |
| Ddx24 | | | Srp19 | | | |
| Klc1 | | | Reep5 | | | |
| Ghr | | | Dpysl3 | | | |
| Basp1 | | | Ap3s1 | | | |
| Mtdh | | | Ppic | | | |
| Plec | | | Gm16286 | | | |
| Rps19bp1 | | | Txnl4a | | | |
| Desi1 | | | Gstp1 | | | |
| Tspo | | | Prdx5 | | | |
| Slc48a1 | | | Fam111a | | | |
| Fkbp11 | | | Ak3 | | | |
| Comt | | | | | | |
| Vps8 | | | | | | |
| Lpp | | | | | | |
| Ccdc50 | | | | | | |
| Senp5 | | | | | | |
| Ccdc80 | | | | | | |
| Phldb2 | | | | | | |
| Cldnd1 | | | | | | |
| App | | | | | | |
| Tnfrsf12a | | | | | | |
| Uqcc2 | | | | | | |
| Slc39a7 | | | | | | |
| Ppp1r18 | | | | | | |
| Myl12a | | | | | | |
| Lbh | | | | | | |
| Cyp1b1 | | | | | | |
| Mcfd2 | | | | | | |
| Slc39a6 | | | | | | |
| Bin1 | | | | | | |
| Egr1 | | | | | | |
| Smim3 | | | | | | |
| Tubb6 | | | | | | |
| 1810055G02Rik | | | | | | |
| Fosl1 | | | | | | |
| Neat1 | | | | | | |
| Rps6ka4 | | | | | | |
| Ppp1r14b | | | | | | |
| Ahnak | | | | | | |
| Fth1 | | | | | | |
| Ccdc86 | | | | | | |
| Anxa1 | | | | | | |
| Acta2 | | | | | | |
| Myof | | | | | | |
| Tm9sf3 | | | | | | |

In particular, regulatory analysis identified a series of TFs that were upregulated in cells along the trajectory to iPSCs and predictive of the expression of the pluripotency programs (FIG. 26D). The earliest predictive TFs were expressed at day 9 (including Nanog, Sox2, Mybl2, Elf3, Tgif1, Klf2, Etv5, and Cdc51) and additional predictive TFs were induced at day 10 (including Klf4, Esrrb, Spic, Zfp42, Hesx1, and Msc). Of these 14 TFs, 9 had previously described roles in regulation of pluripotency (Nanog, Sox2, Mybl2, Klf2, Cdc51, Klf4, Esrrb, Zfp42, and Hesx1) (Aaronson et al., 2016; Boheler, 2009; Buganim et al., 2012; Hu et al., 2009; Jeon et al., 2016; Li et al., 2015; Shi et al., 2006). A further wave of predictive TFs was upregulated in the iPSC trajectory between day 12 and 14, including Obox6, Sohlh2, Ddit3, and Bhlhe40. Among these late TFs, Obox6 and Sohlh2 were particularly notable, because they were not induced in the trajectories to any other cell fate. Obox6 and Sohlh2 had not previously been reported to be involved in regulation of pluripotency, but both had been implicated in maintenance and survival of germ cell development (Park et al., 2016; Rajkovic et al., 2002).

An important change known to occur in the late stages of successful reprogramming was the reversal of X-chromosome inactivation in female cells. Our trajectory analysis identified the correct order of events as previously reported, but without the need for specialized experiments. Specifically, a study based on microscopy of cells labeled with antibodies to specific pluripotency proteins and RNA FISH for Xist (Pasque et al., 2014) showed that Xist downregulation preceded X-chromosome reactivation and positioned these events relative to the appearance of four pluripotency-associated proteins in Nanog-positive cells. Consistently, in our model, along the trajectory to successful reprogramming (but not elsewhere), cells at day 10 showed strong downregulation of Xist but did not yet display a signature of X-reactivation (FIGS. 26E, 26F, Methods). X-reactivation was complete at day 18, with the signature score having risen from 1.05 at day 10 to −1.95 at day 18, consistent with the expected increase in X-chromosome expression (FIG. 26F) (Pasque et al., 2014).

Development of Extra-Embryonic-Like Cells During Reprogramming

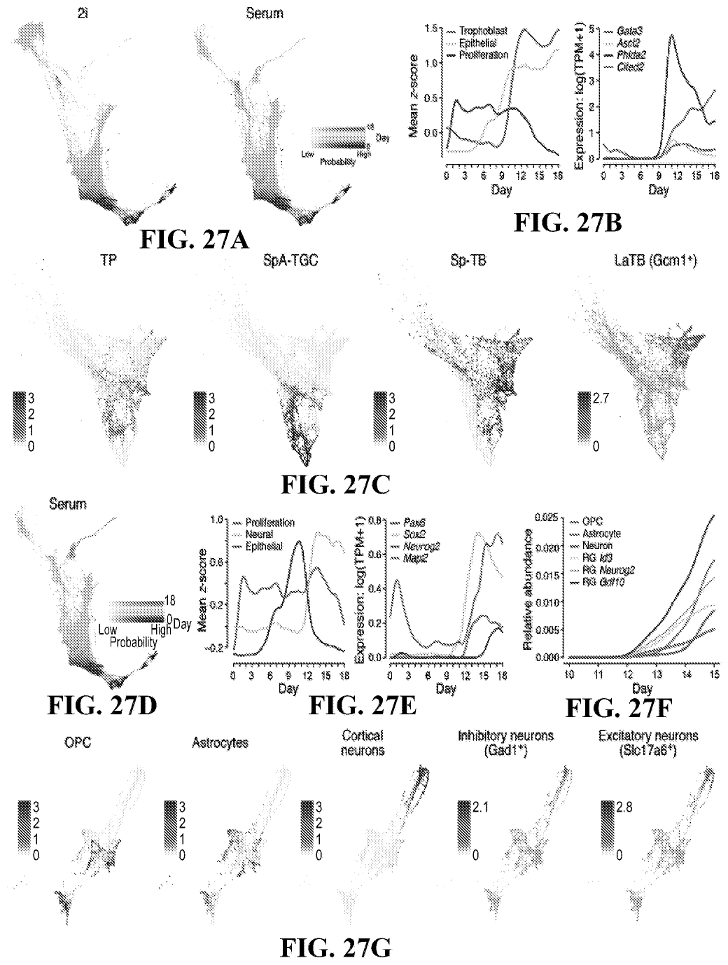

Our trajectories showed that another subset of cells emerges from the MET Region, gained a strong epithelial signature by day 9, and went on to express a clear trophoblast signature (FIG. 27A, 27B). The trophoblast signature was detectable by day 10.5 and peaked by day 12.5, when such cells accounted for ˜20% of all cells in both serum and 2i conditions (FIG. 24G). Trophoblast and pre-implantation programs had previously been observed late in human reprogramming (Cacchiarelli et al., 2015)

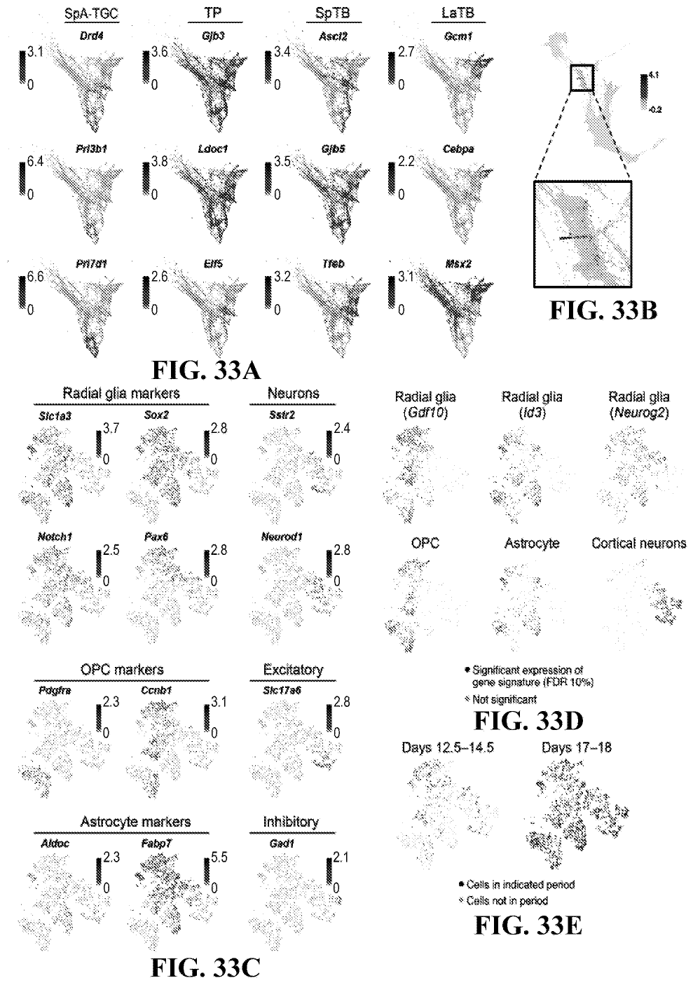

The cells spanned a spectrum of developmental programs associated with specific trophoblasts subsets. Briefly, in normal development the extraembryonic trophoblast progenitors (TPs) gave rise to the chorion, which formed labyrinthine trophoblasts (LaTBs), and the ectoplacental cone, which gave rise to various types of spongiotrophoblasts (SpTBs) and trophoblast giant cells (TGCs), including spiral artery trophoblast giant cells (SpA-TGCs). We scored our cells with signatures we derived from placental scRNA-seq (Nelson et al., 2016) for TP, SpT, TG and SpA-TGCs (Table 15), as well as three well-characterized markers (Msx2, Gcm1 and Cebpa) of LaTBs (Simmons et al., 2008; Ueno et al., 2013), for which no data were available to derive signatures (FIG. 33A). A substantial number of cells expressed TP, SpTB or SpATG signatures in serum conditions and TP or SpTB signatures in 2i conditions, at 10% FDR (FIG. 5C). We also observed a cluster of ˜200 trophoblasts cells that expressed the three LaTBs markers (in 2i but not serum), which were largely separate from those expressing signatures of ectoplacental derivatives. In addition to trophoblast-like cells, ˜125 cells expressed a signature (Lin et al., 2016) for the primitive endoderm (XEN-like cells), the other cell type that contributes to extraembryonic tissue (FIG. 33B, FDR 0.1%). Notably, these cells were seen only in a single replicate at a single time point (day 15.5) in serum conditions only. Two previous studies reported the generation of XEN-like cells during OKSM-induced reprogramming to iPSCs (Parenti et al., 2016, Zhao et al., 2018).

Regulatory analysis associated various TFs with the trajectory from the MET Region to the overall set of trophoblasts (FIG. 27B). TFs at day 10.5 that were predictive of subsequent trophoblast fates included several involved in trophoblast self-renewal (Gata3, Elf5, Mycn, Mybl2) (Kidder and Palmer, 2010) and early trophoblast differentiation (Ovol2, Ascl2) (Latos and Hemberger, 2016), as well as others expressed in trophoblasts but without known roles in trophoblast differentiation (Rhox6, Rhox9, Batf3 and Elf3).

Trajectory and regulatory analysis also identified TFs that were predictive of specific cell subsets. Ancestors of cells with the TP signature expressed Gata3, Pparg, Rhox9, Myt1l, Hnf1b, and Prdm11. Gata3 was involved for trophoblast progenitor differentiation (Ralston et al., 2010) and Pparg was involved for trophoblast proliferation and differentiation of labyrinthine trophoblasts (Parast et al., 2009). The other TFs were known to be expressed in placenta, but their roles in cellular differentiation had not been well characterized. Ancestors of cells with the SpTB or LaTB signature expressed Gata2, Gcm1, Msx2, Hoxd13, and Nr1h4. Gata2 was known to be involved for regulation of specific trophoblast programs (Ma et al., 1997). Gcm1 and Msx2 had specific roles in LaTB differentiation, EMT and trophoblast invasion (Liang et al., 2016; Simmons and Cross, 2005), respectively. Nr1h4 was detected in placental tissue, but its role in trophoblast differentiation had not been characterized. Ancestors of cells with the SpA-TGC signature expressed Hand1, Bbx, Rhox6, Rhox9, and Gata2. Hand1 was known to be necessary for trophoblast giant cell differentiation and invasion (Scott et al., 2000). Bbx was a core trophoblast gene known to induced by upstream TFs Gata3 and Cdx2 (Ralston et al., 2010) (FIGS. 33A-33E).

Neural-like cells also emerged from the MET Region during reprogramming in serum conditions.

Only in serum conditions, a third subset of cells emerged from the MET Region, gained a strong epithelial signature, and went on to develop clear neural signatures (FIGS. 27D-27F). These cells were not seen in 2i conditions, presumably due to the differentiation inhibitors in this condition. Compared to the trophoblast-like cells, the signature for neural identity emerged more slowly, by roughly two days (FIG. 24G). The ancestors of neural like cells diverged from the ancestors of trophoblasts and iPSCs by day 9 (FIG. 26B), and then underwent a rapid transition at day 12.5, losing their epithelial signatures and gaining neural signatures (FIGS. 27D, 27E). The signature was maintained through day 18, when such cells comprised 21.5% of all cells in serum conditions.

In normal neural development, neuroepithelial cells lost their epithelial identity and upregulated glial factors, transforming into radial glial cells (Florio and Huttner, 2014; Ming and Song, 2011). Radial glial cells gave rise to astrocytes and oligodendrocytes, and in the CNS also served as progenitors for many neurons (Ming and Song, 2011). To probe these identities, we used scRNA-Seq data from mouse brain to derive signatures that distinguished different cell types and differentiation states (Table 15). These included signatures of (i) astrocytes, oligodendrocyte precursor cells (OPCs), and neurons in adult brain from in the Allen Brain Atlas (http://www.brain-map.org), and (ii) three unlabeled clusters of radial glial cells in E18 mouse brain (Han et al., 2018), each distinguished by high expression of a different gene (Id3, Gdf10, and Neurog2, respectively).

Cells in the landscape spanned multiple stages of neuronal differentiation. Cells near the base of the “neural spike” in the landscape (day 12.5-18) expressed radial glial and neural stem-cell markers (including Pax6 and Sox2) and cells further out along the spike (day 15-18) expressed markers of neuronal differentiation (including Neurog2 and Map2. About 70% of the neural-like cells had significant expression (at 10% FDR) of at least one of the six signatures (FIG. 27G). Cells with the three radial glial signatures appeared first, concurrent with the loss of epithelial identity and first gained of neural lineage identity by day 12.5 (FIG. 27F). Cells expressing the signatures derived from adult neurons and glia emerged around day 14 in the neural spike and grew in abundance for the duration of the time course. Their ancestors were concentrated in the radial glial populations on day 13.5, with a particular concentration in the Gdf10 RG subpopulation. While the glial populations overlapped substantially, the neurons form a distinct population with substantial substructure. The subset of cells with signatures of adult neurons included cells with canonical markers for excitatory and inhibitory neurons (Slc17a6 and Gad1, respectively). Expression signatures that distinguished these two classes of cells showed strong, albeit incomplete, overlapped with respective programs of excitatory and inhibitory neurons in the Allen Brain Atlas (FIG. 27G, Methods).

Regulatory analysis identified TFs predictive of the overall neural-like cell population, with the top TFs all known to have roles in various stages of neurogenesis. These TFs included those known to promote early neurogenesis (Rarb, Foxp2, Emx1, Pou3f2, Nr2f1, Myt1l, Neurod4), regulated late neurogenesis (Scrt2, Nhlh2, Pou2f2), regulated differentiation and survival of neural subtypes (Onecut1, Tal2, Barhl1, Pitx2), and played roles in neural tube formation (Msx1, Msx3).

The Developmental Landscape Highlighted Potential Paracrine Signals

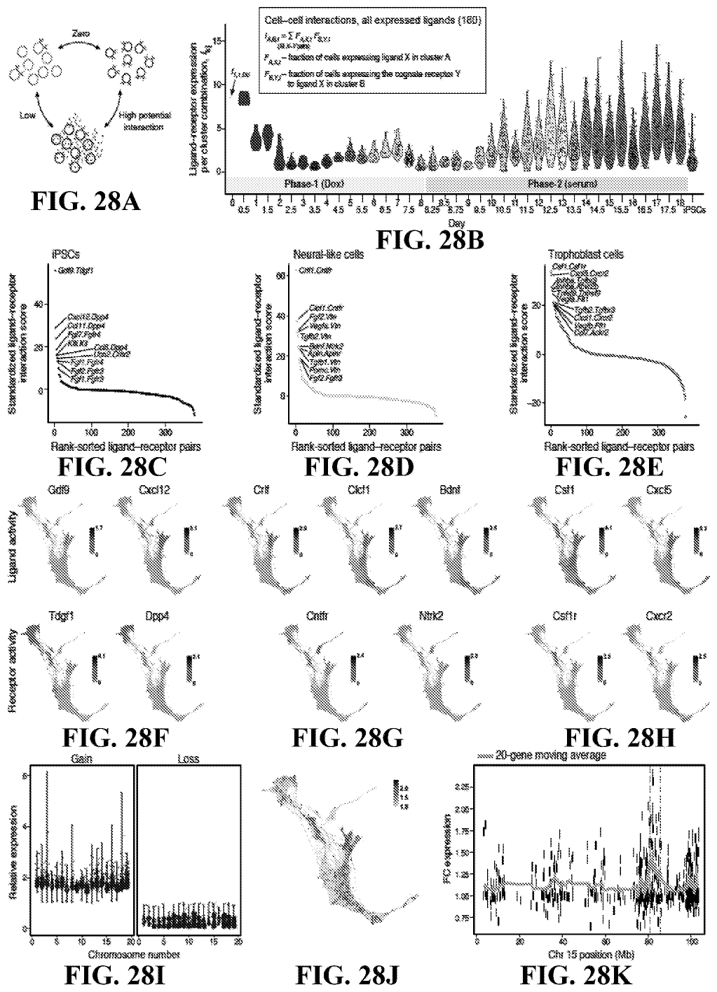

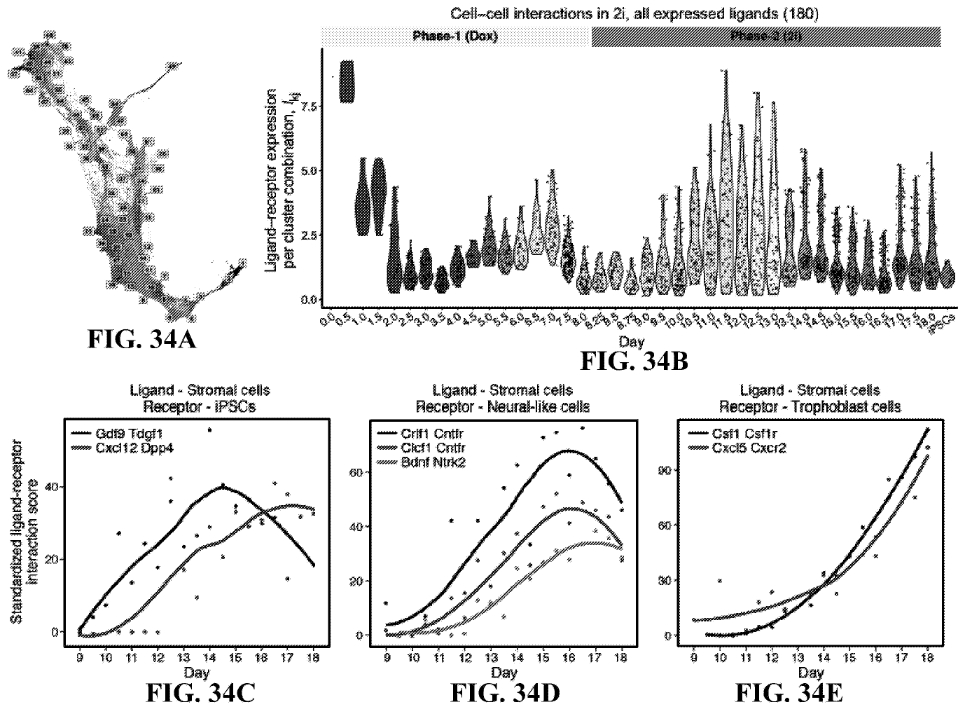

As the reprogramming landscape included a substantial and under-appreciated diversity of differentiating cell subsets, including stromal, epithelial, neural and trophoblast cells, we asked how they might affect each other as they undergo dynamic processes concurrently. In particular, paracrine signaling played a key role in normal development and had also been shown to affect reprogramming, with secretion of inflammatory cytokines enhancing reprogramming efficiency (Mosteiro et al., 2016). Accordingly, we systematically cataloged the contemporaneous occurrence of ligand-receptor pairs across cell subsets in the developmental landscape. We defined an interaction score based on the product of (1) fraction of cells of type A expressing ligand X and (2) the fraction of cells of type B expressing the cognate receptor Y, at the same time t (FIGS. 28A, 28B and 34B, Methods). We examined 180 individual cognate ligand-receptor pairs, as well as an aggregate score across all pairs between cell clusters (FIG. 34A) and across those pairs related to the SASP signature.

The landscape revealed rich potential for paracrine signaling (FIG. 28B, FIG. 34B, Table 18). In particular, we observed high interaction scores for several SASP ligands in stromal cells with receptors expressed in iPSCs, such as Gdf9 with Tdgf1 (Polo et al., 2012) and Cxcl12 with Dpp4 (FIGS. 28C, 28F, 34C).

TABLE 18
Potential ligand-receptor pairs between stromal cells and iPSCs, neural-
like cells, and trophoblast cells ranked by standardized interaction scores

| Ligand: Stromal cells, | | | Ligand: Stromal cells, | | | Ligand: Stromal cells, | | |
|---|---|---|---|---|---|---|---|---|
| Receptor: | | | Receptor: | | | Receptor: | | |
| iPSCs | | | Neural-like cells | | | Trophoblast cells | | |
| | Maximal | | | Maximal | | | Maximal | |
| Ligand- | standardized | Peak | Ligand- | standardized | Peak | Ligand- | standardized | Peak |
| Receptor | interaction | Score | Receptor | interaction | Score | Receptor | interaction | Score |
| Pair | score | Day | Pair | score | Day | Pair | score | Day |
| Gdf9.Tdgf1 | 55.83015277 | 14 | Crlf1.Cntfr | 76.16064491 | 16.5 | Csf1.Csf1r | 111.8151997 | 18 |
| Cxcl12.Dpp4 | 42.40247659 | 12.5 | Fgf2.Vtn | 66.31283077 | 18 | Cxcl5.Cxcr2 | 102.1031447 | 18 |
| Ngf.Ngfr | 26.79815659 | 12 | Clcf1.Cntfr | 52.04021271 | 15.5 | Cxcl1.Cxcr2 | 85.46017232 | 18 |
| Ccl11.Dpp4 | 23.75254375 | 14 | Vegfa.Vtn | 39.99828338 | 18 | Il6.Il6ra | 70.79780689 | 18 |
| Kitl.Kit | 20.48156022 | 17.5 | Bdnf.Ntrk2 | 38.24132006 | 17 | Cxcl2.Cxcr2 | 68.04261554 | 18 |
| Ccl5.Dpp4 | 20.22465038 | 12.5 | Tgfb2.Vtn | 37.9492686 | 18 | Cxcl3.Cxcr2 | 62.67646817 | 17.5 |
| Inhba.Acvr2b | 18.91224205 | 17 | Tgfb1.Vtn | 37.71506462 | 18 | Il7.Il2rg | 57.89558657 | 17 |
| Fgf7.Fgfr4 | 18.88448993 | 12 | Tgfb3.Tgfbr1 | 32.86035119 | 17 | Vegfa.Flt1 | 52.30228603 | 18 |
| Nppc.Npr1 | 17.71660947 | 16.5 | Bdnf.Sort1 | 29.14910223 | 17 | Tg.Lrp2 | 45.35387653 | 9.5 |
| Fgf7.Fgfr2 | 17.2915253 | 9 | Il16.Grin2a | 27.83837935 | 13.5 | Ccl2.Ackr2 | 44.70456305 | 17 |
| Grn.Cry1 | 17.25111965 | 17 | Inhba.Acvr2b | 25.85377693 | 15.5 | Spp1.Itgb1 | 44.39437623 | 18 |
| Fgf2.Fgfr3 | 17.18398331 | 15.5 | Apln.Aplnr | 23.46381586 | 14 | Il15.Il2rg | 43.96702273 | 18 |
| Spp1.F2 | 16.91745599 | 17 | Bmp1.Adra1a | 21.99556814 | 17.5 | Ccl7.Ackr2 | 42.35095481 | 17 |
| Tgfb3.Tgfbr1 | 15.80306191 | 9 | Il16.Grin2b | 21.85263644 | 18 | Tnfsf9.Tnfrsf9 | 41.80288631 | 15.5 |
| Bdnf.Ntrk2 | 15.73929703 | 12 | Vegfa.Ephb2 | 21.76727834 | 17 | Cxcl15.Cxcr2 | 41.37975891 | 18 |
| Avp.Avpr1b | 15.6652861 | 15 | Tgfb1.Tgfbr1 | 21.71078611 | 17 | Vegfb.Flt1 | 40.59359924 | 18 |
| Inhbb.Acvr2b | 15.22902239 | 18 | Ngf.Sort1 | 21.55867193 | 16.5 | Fgf2.Fgfr1 | 40.1892017 | 18 |
| Tnfsf8.Tnfrsf8 | 14.9661866 | 17.5 | Ereg.Erbb4 | 21.23888338 | 17 | Il15.Il2rb | 37.23349427 | 18 |
| Ucn2.Crhr2 | 14.66104887 | 14 | Cxcl12.Cxcr4 | 20.66598418 | 16.5 | Il2.Il2rg | 34.72049417 | 17 |
| Sst.Sstr3 | 14.53946813 | 12.5 | Nov.Notch1 | 20.64844205 | 17 | Il1rn.Il1r2 | 34.60876011 | 18 |
| Cxcl12.Cxcr4 | 13.99702972 | 9.5 | Inhbb.Acvr2b | 20.20541981 | 15.5 | Bmp4.Bmpr2 | 33.37381523 | 18 |
| Fgf1.Fgfr4 | 13.23808582 | 14 | Egf.Vtn | 20.11367671 | 14.5 | Ppbp.Cxcr2 | 33.31119733 | 17 |
| Gdf6.Bmpr1b | 13.23695383 | 11.5 | Fgf7.Fgfr2 | 19.85021209 | 9 | Flt3l.Flt3 | 31.32026205 | 17 |
| Gdf9.Bmpr1b | 12.81536347 | 11.5 | Fgf10.Fgfr2 | 19.77063453 | 12 | Inhba.Acvr2b | 31.21420166 | 16.5 |
| Gdf5.Acvr2b | 12.41295756 | 17.5 | Fgf2.Fgfr3 | 19.20901825 | 18 | Il2.Il2rb | 31.17852066 | 17 |
| Cxcl3.Cxcr2 | 12.28144255 | 9 | Inhba.Igsf1 | 19.00415822 | 13.5 | Inhbb.Acvr1b | 31.08869402 | 18 |
| Cxcl10.Dpp4 | 12.0118101 | 16.5 | Pomc.Vtn | 18.61879864 | 14 | Inhba.Acvr1b | 30.95069812 | 18 |
| Tnfsf11.Tnfrsf11a | 11.98501062 | 18 | Tgfb2.Tgfbr1 | 18.40997602 | 17 | Ccl8.Ackr2 | 30.92303758 | 17 |
| Tnfsf11.Med24 | 11.31495458 | 17 | Gdf9.Tdgf1 | 18.12847923 | 10.5 | Pgf.Flt1 | 28.55965416 | 17 |
| Bdnf.Inpp5k | 11.02760154 | 17 | Gdnf.Gfra1 | 17.94758176 | 18 | Tgfb3.Tgfbr1 | 28.48415966 | 18 |
| Cxcl5.Cxcr2 | 10.76725496 | 9 | Edn1.Ednrb | 17.81157803 | 17 | Inhba.Tgfbr3 | 27.97080183 | 18 |
| Bmp2.Bmpr1b | 10.52856679 | 11.5 | Gdf11.Acvr2b | 16.93911315 | 15.5 | Inhbb.Acvr2b | 27.64710304 | 18 |
| Inhba.Acvr1b | 10.45689595 | 15.5 | Gdf5.Bmpr1b | 16.87028377 | 17 | Ccl3.Ackr2 | 27.17947452 | 14.5 |
| Fgf1.Fgfr3 | 9.904359216 | 14 | Gdf5.Acvr2b | 16.68587549 | 15.5 | Tgfb3.Sdc4 | 26.70563028 | 18 |
| Tgfb3.Eng | 9.606914311 | 18 | Igf1.Igf1r | 16.40043325 | 17.5 | Inhba.Acvrl1 | 24.8733331 | 16.5 |
| Crlf1.Cntfr | 9.491489628 | 9 | Ngf.Ngfr | 16.1554284 | 9 | Wnt5a.Fzd5 | 24.08669584 | 18 |
| Tg.Lrp2 | 9.311152429 | 9.5 | Cxcl5.Ackr1 | 15.81074369 | 17 | Egf.Erbb3 | 22.88090865 | 18 |
| Nppa.Nr5a2 | 9.196846339 | 15.5 | Tg.Lrp2 | 15.56587296 | 9.5 | Gdf5.Acvr2b | 22.79535492 | 16.5 |
| Spp1.Itgb1 | 9.094293313 | 9 | Il16.Kcnj10 | 15.40280917 | 15 | Tgfb1.Itgb6 | 22.73325122 | 18 |
| Tgfb3.Sdc4 | 8.962618473 | 18 | Ccl2.Ackr1 | 14.80314224 | 17 | Vegfc.Flt4 | 22.64781847 | 18 |
| Avp.Avpr2 | 8.816318411 | 16 | Il1rn.Il1r2 | 14.70537108 | 17 | Vegfa.Kdr | 21.61880314 | 13 |
| Bmp4.Bmpr1b | 8.789458439 | 11.5 | Wnt5a.Fzd2 | 14.59368545 | 16.5 | Il18.Il18rap | 21.45320636 | 18 |
| Gdf11.Acvr2b | 8.657009643 | 17.5 | Inhbb.Igsf1 | 14.56070266 | 13.5 | Tgfb2.Tgfbr3 | 21.43696896 | 12.5 |
| Ctgf.Egfr | 8.474450513 | 9 | Ccl12.Ackr1 | 14.48343455 | 15 | Fgf7.Fgfr2 | 21.27556999 | 9 |
| Nov.Notch1 | 7.853128492 | 9.5 | Ccl7.Ackr1 | 14.45732094 | 17 | Ccl12.Ackr2 | 20.65465765 | 15 |
| Cxcl1.Cxcr2 | 7.825570863 | 9 | Fgf1.Fgfr3 | 13.98128161 | 14 | Tgfb1.Tgfbr3 | 19.07802333 | 18 |
| Pomc.Mc5r | 7.803289928 | 13 | Cort.Sstr2 | 13.83366019 | 14.5 | Ccl11.Ackr2 | 19.06812091 | 16.5 |
| Inhba.Acvr2a | 7.697312114 | 10 | Vegfa.Kdr | 13.52841955 | 17 | Ccl28.Ackr2 | 19.0608243 | 16.5 |
| Il16.Cd4 | 7.691300029 | 16 | Bmp4.Bmpr1b | 13.17024743 | 17 | Kitl.Kit | 18.32774459 | 10 |
| Hcrt.Npffr2 | 7.611421106 | 14.5 | Igf1.Igsf1 | 13.1615924 | 13.5 | Gdf11.Acvr2b | 17.1611013 | 16.5 |
| Nppa.Npr1 | 7.327171012 | 15.5 | Inhba.Acvr2a | 12.86079359 | 15.5 | Bdnf.Inpp5k | 16.94541624 | 18 |
| Fgf2.Fgfr1 | 6.935257539 | 18 | Gdnf.Gfra2 | 12.82585678 | 18 | Ccl5.Ackr2 | 16.65970084 | 10.5 |
| Inhbb.Acvr1b | 6.8878958 | 15.5 | Ntf3.Ntrk2 | 12.69375513 | 14 | Ngf.Ngfr | 16.41502139 | 9 |
| Ccl17.Ccr4 | 6.846358767 | 17 | Cxcl1.Ackr1 | 12.64243264 | 17 | Igf1.Igf1r | 16.27850014 | 18 |
| Il16.Grin2b | 6.789839819 | 14.5 | Fgf2.Fgfr1 | 12.31083274 | 18 | Bmp2.Bmpr2 | 15.99972954 | 18 |
| Bdnf.Sort1 | 6.67375428 | 9 | Vegfa.Nrp2 | 12.23441434 | 18 | Tgfb1.Acvrl1 | 15.96504429 | 16.5 |
| Tgfb2.Tgfbr1 | 6.519268162 | 9 | Bmp6.Acvr2b | 12.1758211 | 13.5 | Gdf5.Bmpr2 | 15.58998037 | 16.5 |
| Ntf3.Ntrk2 | 6.438685726 | 12 | Hbegf.Erbb4 | 12.00500039 | 14.5 | Tgfb2.Tgfbr1 | 15.53065603 | 18 |
| Ccl3.Ccr5 | 6.407610415 | 12.5 | Vegfc.Kdr | 11.97527882 | 18 | Tgfb1.Tgfbr1 | 15.49109459 | 18 |
| Ptn.Plxnb2 | 6.364004505 | 9 | Ccl17.Ackr1 | 11.93535268 | 16 | Inha.Tgfbr3 | 14.94814105 | 18 |
| Egf.Erbb3 | 6.33209249 | 17 | Cxcl3.Cxcr2 | 11.79741482 | 9 | Ccl27a.Ackr2 | 14.35654443 | 17 |
| Fgf9.Fgfr3 | 6.17049013 | 15.5 | Wnt2.Fzd9 | 11.76547196 | 14.5 | Pf4.Ldlr | 13.49144052 | 17.5 |
| Ntf3.Ntrk3 | 6.071479576 | 12.5 | Tnfsf11.Med24 | 11.58428169 | 17 | Vegfc.Kdr | 13.42241254 | 12.5 |
| Wnt5a.Fzd5 | 6.049412152 | 17.5 | Cxcl15.Ackr1 | 11.39063421 | 16 | Fgf10.Fgfr2 | 12.93211376 | 12 |
| Il16.Kcnj4 | 5.956600472 | 9 | Cxcl5.Cxcr2 | 10.81475088 | 9 | Pdgfc.Pdgfra | 12.7181284 | 18 |
| Fgf10.Fgfr2 | 5.735961453 | 10 | Spp1.Itgb1 | 10.57557893 | 9 | Ccl25.Ackr2 | 12.58225578 | 10.5 |
| Csf3.Csf3r | 5.660332275 | 18 | Ccl8.Ackr1 | 10.24654012 | 18 | Crlf1.Cntfr | 12.56270017 | 9 |
| Ngf.Sort1 | 5.631416895 | 9 | Gdf5.Acvr2a | 9.947335355 | 16.5 | Inhba.Acvr1 | 12.49512116 | 18 |
| Wnt2.Fzd9 | 5.625683619 | 13 | Inhbb.Acvr2a | 9.83065505 | 17.5 | Inhbb.Acvr1 | 12.17571989 | 18 |
| Ngf.Ntrk1 | 5.482536008 | 18 | Bmp2.Bmpr1b | 9.823905055 | 17 | Bmp4.Bmpr1a | 12.13592365 | 18 |
| Ccl2.Ccr10 | 5.204305876 | 9 | Ngf.Ntrk1 | 9.765431603 | 15.5 | Hgf.Met | 11.85706092 | 18 |
| Gdf5.Bmpr1b | 5.164323069 | 11.5 | Ctgf.Egfr | 9.510948488 | 9 | Avp.Avpr1b | 11.8443167 | 12.5 |
| Ccl7.Ccr10 | 5.03794601 | 9 | Il16.Grin2c | 9.210664243 | 16.5 | Wnt5a.Lrp6 | 11.2866016 | 18 |
| Inhba.Igsf1 | 4.652799622 | 16.5 | Igf2.Vtn | 9.08515341 | 15.5 | Il1rn.Il1r1 | 11.21386458 | 18 |
| Igf1.Igsf1 | 4.623901723 | 16.5 | Fgf9.Fgfr3 | 8.929720296 | 13 | Npff.Npffr2 | 11.12680175 | 12.5 |
| Kitl.Epor | 4.572546653 | 9 | Ucn2.Crhr2 | 8.529535163 | 10 | Gpi1.Amfr | 11.09557616 | 18 |
| Bmp6.Bmpr1b | 4.21969712 | 11.5 | Gdf9.Bmpr1b | 8.458633534 | 12.5 | Ccl2.Ccr5 | 10.87678026 | 17 |
| Il16.Grin2a | 4.182303182 | 12 | Cxcl1.Cxcr2 | 8.317259429 | 9 | Inhba.Acvr2a | 10.71764165 | 18 |
| Tgfb1.Tgfbr1 | 4.165309406 | 9 | Pnoc.Oprl1 | 8.170486417 | 13 | Inhbb.Acvr2a | 10.62573575 | 18 |
| Hmgb1.Pgr | 4.162814163 | 9.5 | Inha.Acvr2a | 8.005902758 | 15.5 | Ccl17.Ccr4 | 10.22222634 | 11.5 |
| Tnfsf13b.Tnfrsf17 | 4.077062584 | 16.5 | Inhba.Acvr1b | 7.58971181 | 9.5 | Vegfa.Lyve1 | 9.978529316 | 11.5 |
| Il16.Grin2c | 3.818702923 | 17 | Fgf7.Fgfr4 | 7.313765731 | 16 | Lif.Lifr | 9.836393324 | 16.5 |
| Crh.Crhr2 | 3.804963778 | 14 | Ptn.Plxnb2 | 7.174330257 | 9 | Il25.Il17rb | 9.820316363 | 16 |
| Tgfb1.Eng | 3.789167413 | 17 | Btc.Erbb4 | 7.130596933 | 14.5 | Ccl8.Ccr5 | 9.277471947 | 16.5 |
| Ccl5.Ccr5 | 3.765684384 | 10.5 | Grn.Cry1 | 7.038337946 | 16.5 | Il16.Kcnj10 | 9.099847388 | 14.5 |
| Ccl3.Ackr4 | 3.748657973 | 12.5 | Il16.Kcnj2 | 7.031491551 | 18 | Bdnf.Ntrk2 | 9.027486627 | 12.5 |
| Ccl2.Ccr5 | 3.746070011 | 12.5 | Edn1.Ednra | 6.737910303 | 17.5 | Edn1.Ednrb | 8.719812556 | 14 |
| Gdf5.Acvr2a | 3.726614996 | 16 | Avp.Oxtr | 6.701328931 | 16.5 | Cxcl12.Cxcr4 | 8.696493411 | 17 |
| Npff.Npffr2 | 3.71584242 | 14.5 | Tgfb3.Sdc4 | 6.648807091 | 9 | Fgf9.Fgfr1 | 8.617860569 | 18 |
| Inhbb.Igsf1 | 3.660059949 | 16.5 | Il16.Kcnj4 | 6.296091418 | 9 | Spp1.F2 | 8.219496273 | 13.5 |
| Bmp6.Acvr2b | 3.613241885 | 13.5 | Spp1.F2 | 6.250718711 | 14.5 | Ptn.Plxnb2 | 8.085698538 | 9 |
| Lif.Lifr | 3.59302184 | 12.5 | Adm.Calcrl | 6.127364131 | 18 | Tnfsf11.Med24 | 8.080587047 | 18 |
| Inhbb.Acvr2a | 3.573362535 | 16 | Artn.Gfra3 | 6.100580729 | 18 | Ctgf.Egfr | 8.025815916 | 9 |
| Tgfb2.Eng | 3.493150482 | 18 | Ccl5.Ackr1 | 6.08281121 | 16 | Ghrl.Ptger3 | 7.831218363 | 15 |
| Tnfsf13b.Tnfrsf13b | 3.485242199 | 14 | Tgfb3.Eng | 6.075334099 | 9 | Ctf1.Lifr | 7.478421588 | 18 |
| Bmp2.Bmpr1a | 3.421538818 | 9 | Gdf6.Bmpr1b | 5.814695498 | 17.5 | Pdgfd.Pdgfrb | 7.440471865 | 18 |
| Bmp2.Eng | 3.277644443 | 12 | Hmgb1.Pgr | 5.524547346 | 9.5 | Gdf5.Acvr2a | 7.437486529 | 17.5 |
| Pf4.Ldlr | 3.252582504 | 11.5 | Wnt5a.Lrp6 | 5.416442742 | 15 | Cxcl12.Dpp4 | 7.386223592 | 12.5 |
| Ntf5.Ngfr | 3.228481212 | 12 | Vegfa.Lyve1 | 5.365931818 | 16.5 | Ccl11.Ccr5 | 7.344244377 | 16.5 |
| Ccl5.Ccr4 | 3.054614918 | 17 | Ccl17.Ccr4 | 5.313995351 | 9.5 | Gdf5.Bmpr1a | 7.242141121 | 17.5 |
| Pgf.Nrp2 | 3.013909017 | 9 | Sst.Sstr2 | 4.993026408 | 12.5 | Artn.Gfra3 | 6.624252893 | 16 |
| Fgf8.Fgfr4 | 3.01220056 | 14 | Vegfa.Flt1 | 4.860449031 | 13.5 | Il18.Il1rl2 | 6.470340015 | 18 |
| Artn.Gfra3 | 3.008145345 | 16 | Bmp6.Bmpr1b | 4.604550067 | 16.5 | Inha.Acvr2a | 6.410004454 | 18 |
| | | | Egf.Erbb3 | 4.487189494 | 10.5 | Gdf6.Bmpr2 | 6.362677796 | 18 |
| | | | Kitl.Epor | 4.470894246 | 9 | Ntf3.Ntrk2 | 6.34714587 | 12.5 |
| | | | Gdf9.Acvr2a | 4.461925767 | 12.5 | Gdf5.Acvr1 | 6.33836936 | 18 |
| | | | Ccl2.Ccr10 | 4.287535378 | 9 | Tslp.Prnp | 6.263327318 | 18 |
| | | | Fgf9.Fgfr2 | 4.104799154 | 11 | Gdf9.Tdgf1 | 6.170602382 | 10.5 |
| | | | Il16.Cd4 | 4.102677906 | 15.5 | Bdnf.Sort1 | 5.94172272 | 9 |
| | | | Ccl2.Ccr5 | 4.06128803 | 18 | Bmp2.Acvr1 | 5.90978443 | 18 |
| | | | Ntf3.Ntrk1 | 4.045425855 | 15.5 | Bmp6.Acvr2b | 5.871545931 | 13.5 |
| | | | Bmp2.Bmpr1a | 4.007512362 | 9 | Tnfsf11.Tnfrsf11a | 5.868170248 | 15.5 |
| | | | Pdgfc.Pdgfra | 4.000578173 | 18 | Il6.Il6st | 5.857031136 | 18 |
| | | | Bmp4.Bmpr1a | 3.973107083 | 17 | Kitl.Epor | 5.493268145 | 14 |
| | | | Ghrl.Ptger3 | 3.959803347 | 15 | Hmgb1.Pgr | 5.439455664 | 9.5 |
| | | | Il11.Il11ra1 | 3.931542903 | 16.5 | Gdf9.Bmpr2 | 5.301534907 | 17.5 |
| | | | Ccl7.Ccr10 | 3.86216627 | 9 | Ngf.Sort1 | 5.181692923 | 9 |
| | | | Gdf5.Bmpr1a | 3.812514632 | 16.5 | Tnfsf13b.Tnfrsf13b | 5.166928123 | 15.5 |
| | | | Ntf5.Ntrk2 | 3.800422565 | 15.5 | Ucn2.Crhr2 | 5.15524664 | 9 |
| | | | Ntf3.Ntrk3 | 3.791204113 | 13 | Fgf1.Fgfr1 | 5.090269326 | 18 |
| | | | Ccl8.Ccr5 | 3.6877203 | 18 | Pdgfa.Pdgfra | 4.960203778 | 18 |
| | | | Vegfb.Flt1 | 3.67289066 | 13.5 | Fgf7.Fgfr4 | 4.959156503 | 12 |
| | | | Ccl5.Ccr4 | 3.652617678 | 9.5 | Nov.Notch1 | 4.944351734 | 9.5 |
| | | | Inhba.Acvr1 | 3.386360757 | 18 | Bmp2.Bmpr1a | 4.828229043 | 18 |
| | | | Inhbb.Acvr1 | 3.330148881 | 18 | Fgf2.Fgfr3 | 4.718080894 | 13.5 |
| | | | Wnt1.Fzd9 | 3.30422519 | 12.5 | Grn.Cry1 | 4.629614942 | 9 |
| | | | Npff.Npffr1 | 3.243049647 | 16 | Tgfb3.Eng | 4.541775835 | 9 |
| | | | | | | Tnfsf10.Tnfrsf10b | 4.456880919 | 16.5 |
| | | | | | | Hcrt.Hcrtr1 | 4.407762506 | 14.5 |
| | | | | | | Ccl5.Ccr5 | 4.218364077 | 16 |
| | | | | | | Il16.Kcnj4 | 4.184296843 | 9 |
| | | | | | | Ghrl.Ptgir | 4.00490292 | 15 |
| | | | | | | Cxcl16.Cxcr6 | 3.995533009 | 18 |
| | | | | | | Ccl3.Ccr5 | 3.825939759 | 12.5 |
| | | | | | | Il16.Grin2c | 3.804620341 | 14 |
| | | | | | | Ccl5.Ccr4 | 3.700028296 | 13 |
| | | | | | | Il17b.Il17rb | 3.43715641 | 10.5 |
| | | | | | | Hmgb1.Ar | 3.425935882 | 11 |
| | | | | | | Ntf3.Ntrk1 | 3.384388196 | 13 |
| | | | | | | Ngf.Ntrk1 | 3.213785377 | 13 |
| | | | | | | Ccl12.Ccr5 | 3.032941015 | 16 |

Analysis of the neural-like cells revealed particularly interesting interaction scores involving Cntfr (FIGS. 28D, 28G, 34D), an I16-family co-receptor whose activation played critical roles in neural differentiation and survival (Elson et al., 2000; Nakashima et al., 1999). On day 11.5 in serum conditions, one day before the early neuronal signatures appear, neural ancestors upregulated expression of Cntfr; expression was 4.6-fold higher in epithelial cells that were neural ancestors versus those that were not. Just before, on day 10.5, stromal cells began expressing three activating ligands for Cntfr (Crlf1, Lif, Clcf1). We speculated that these events may help trigger the program of neural differentiation among a subset of epithelial cells in serum conditions. The analysis also revealed a potential interaction involving the ligand-receptor pair Bdnf-Ntrk2, which had been implicated in promoting neuronal development, maturation and survival (Chen et al., 2015; Jukkola et al., 2006; Yun et al., 2008) (FIGS. 28D, 28G, 34D). The same ligand-receptor interactions were seen in 2i conditions, but the MEK inhibitor in 2i medium would be expected to block Cntfr signaling and subsequent neural differentiation.

Trophoblast-like cells also showed notable interaction scores, including Csf1 and Csf1r (FIGS. 28E, 28H). In early placental development, Csf1 was expressed in maternal columnar epithelial cells and Csf1r was expressed in fetal trophoblasts, suggesting a functional role of this interaction in trophoblast development and differentiation. Many of the other top-ranked interactions were between a single receptor in trophoblast cells (Cxcr2) and multiple members of the same ligand family (Cxcl5, Cxcl1, Cxcl2, Cxcl3, and Cxcl15) (FIGS. 24E, 24H, 34E). Cxcr2 had been shown to be necessary for trophoblast invasion in human trophoblast cells (Vandercappellen et al., 2008; Wu et al., 2016).

RNA Expression Revealed Genomic Aberrations in Stromal and Trophoblast-Like Cells

We hypothesized that some cell types might harbor detectable genomic aberrations. In particular, trophoblasts were known to undergo endocycles of replication in vivo (Edgar et al., 2014), resulting in selective amplification of specific genomic regions containing functionally important genes (Hannibal and Baker 2016). Additionally, our stromal cells exhibited signs of stress and cell death which may be associated with genomic aberrations.

To identify potential genomic aberrations, we scored the scRNA-Seq data for large regions showing coherent increases or decreases in gene expression, following successful approaches we developed to identify aberrant regions in individual tumor cells in a patient (Patel et al., 2014). We searched copy-number variations at the level of whole chromosomes and subchromosomal regions spanning 25 consecutive housekeeping genes (median size 25 Mb) (STAR Methods). To evaluate the detection of subchromosomal events, we analyzed scRNA-Seq data from oligodendroglioma (Tirosh et al. 2016): the method had high specificity, but sensitivity to detect only about one-third of events.

Whole-chromosome aneuploidies were detected in 4.0% of trophoblast cells and 2.1% of stromal cells, compared to only 1.1% of all other cells across the landscape. Most whole-chromosome events were consistent with loss or gain of a single copy of the chromosome (FIG. 28I). Subchromosomal events were detected in 6.9% of trophoblast cells and 3.2% of stromal cells, compared to only 1.2% in most other cells types and 0.4% in neural cells (FIG. 6J); the true proportions are likely to be about 3-fold higher, given the estimated sensitivity.

Trophoblast-like cells showed recurrent events at a higher frequency than stromal cells. Among trophoblast cells harboring aberrations, 8.6% were detected as carrying a recurrent event involving apparent duplication (50% higher expression) of a region containing 74 genes (FIG. 28K). Among the genes are Wnt7b, which was required for normal placental development (Parr et al., 2001); Prr5, which mediates Pdfgb signaling required for development of labyrinthine cells (Ohlsson et al., 1999; Woo et al., 2007); and several genes identified as ‘core trophoblast genes’ (Cyb5r3, Cenpm, Srebf2, and Pmm1). The top 15 recurrent events also included the amplification of the prolactin gene cluster on chromosome 13 in 1% of cells. These observations suggested that the trophoblast-associated mechanisms of genomic alteration may be expressed, to some extent, in our trophoblast-like cells.

In the stromal cells with evidence of genomic aberration, the most common recurrent events had lower frequency. Notably, however, the most frequently amplified region contained cell cycle inhibitors Cdkn2a, Cdkn2b, and Cdkn2c, while the most frequently lost region contained Cdk13, which promotes cell cycling, and Mapk9, loss of which promotes apoptosis. These observations suggested that genomic alterations in these regions may contribute to development stromal cells.

Forced Expression of Obox6 Enhanced Reprogramming

Finally, we explored whether some of the new TFs identified by regulatory analysis along the trajectory to iPSCs might provide ways to increase reprogramming efficiency. In principle, TFs could increase the efficiency of reprogramming in several ways, including increasing the transition frequency to iPSC precursors, boosting the growth rate of iPSC precursors, reducing alternative fates of other epithelial-related fates, or increasing supportive paracrine signaling from non-iPS cells.

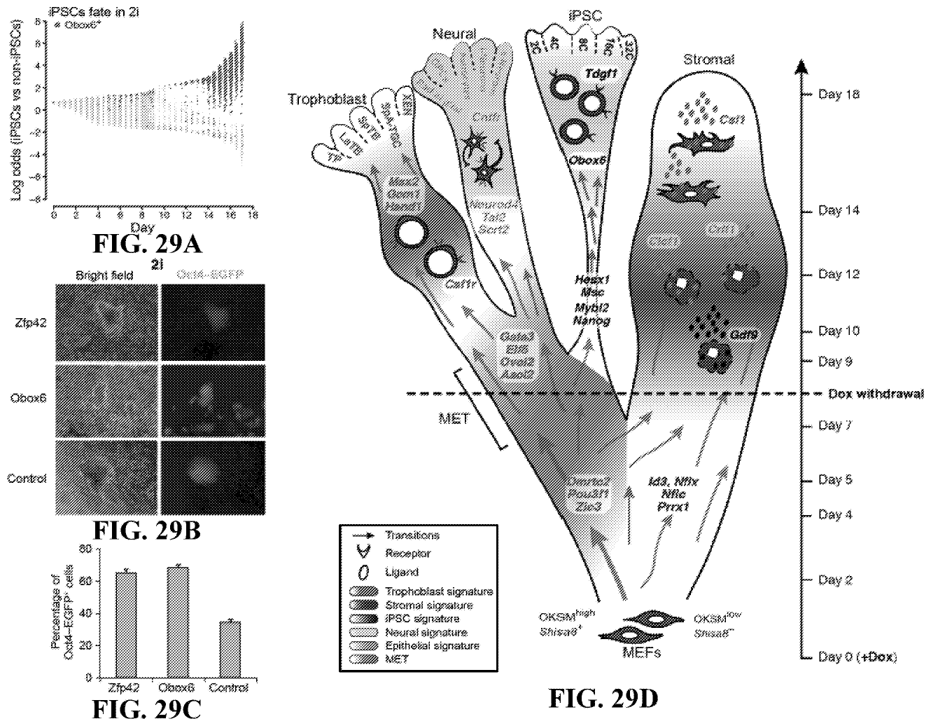

We focused on Obox6, which our regulatory analysis discovered as the TF most strongly correlated with reprogramming success, among those not previously implicated in the process. Obox6 (oocyte-specific homeobox 6) is a homeobox gene of unknown function that is preferentially expressed in the oocyte, zygote, early embryos and embryonic stem cells (Rajkovic et al., 2002). (Although Obox6 was the only Obox family member detected in our experiment, we note that a better-studied oocyte-specific homeobox Obox1 has been shown to enhance reprogramming efficiency, promote MET, and be able to substitute for Sox2 in reprogramming (Wu et al., 2017)). While Obox6 was expressed only in a small fraction of cells (<1%) before day 12, cells expressing Obox6 during day 5.5 to day 8 are highly biased toward the MET Region, with 94% being in the top 50% of cells with respect to the proportion of descendants in this region (FIG. 29A).

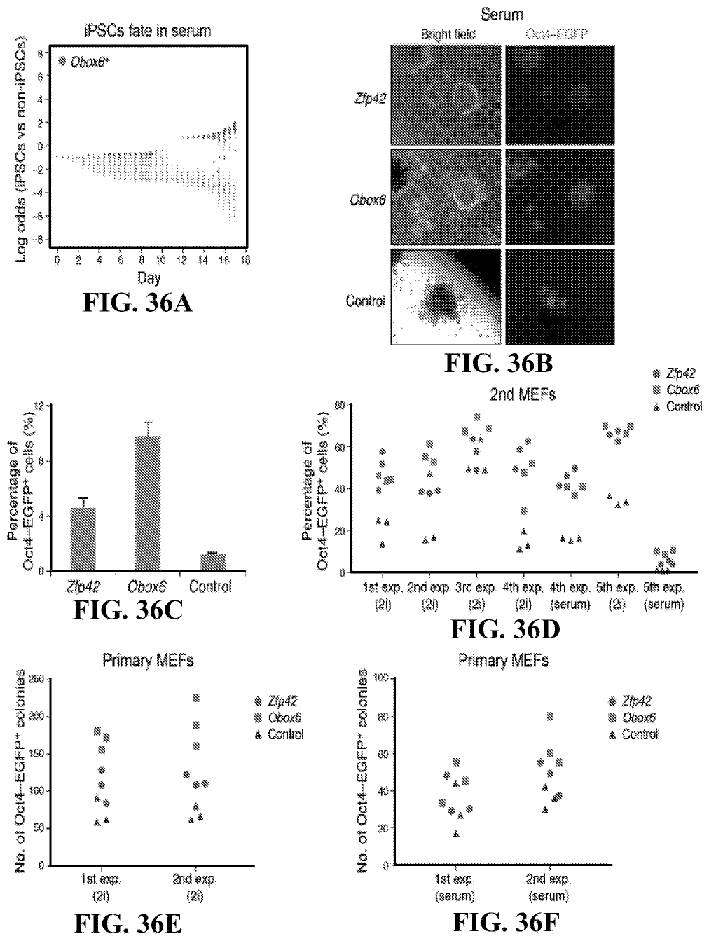

We tested whether expressing Obox6 together with OKSM during days 0-8 can boost reprogramming efficiency. We infected our secondary MEFs with a Dox-inducible lentivirus carrying either Obox6, the known pluripotency factor Zfp42 (Rajkovic et al., 2002; Shi et al., 2006), or no insert as a negative control. Both Obox6 and Zpf42 increased reprogramming efficiency of secondary MEFs by ˜2-fold in 2i and even more so in serum, with the result confirmed in multiple independent experiments (FIGS. 29B, 29C, and 36A-36F). Assays in primary MEFs showed similar increases in reprogramming efficiency (FIGS. 26A-36F).

Together, these computational and experimental results suggested that the role of Obox6 in reprogramming merits further study.

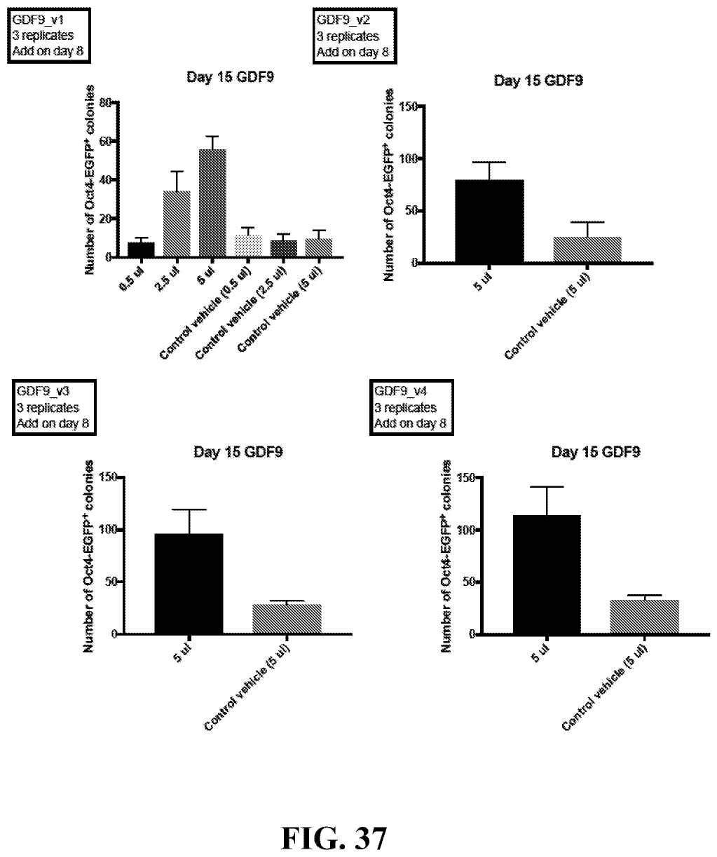

In addition, we identified GDF9 that can significantly booster reprogramming efficiency. We added GDF9 to the medium from day 8. We observed more Oct4-GFP positive colonies (iPSCs) (FIG. 37). We also confirmed that we saw more iPSCs after adding GDF9 by scRNA sequencing.

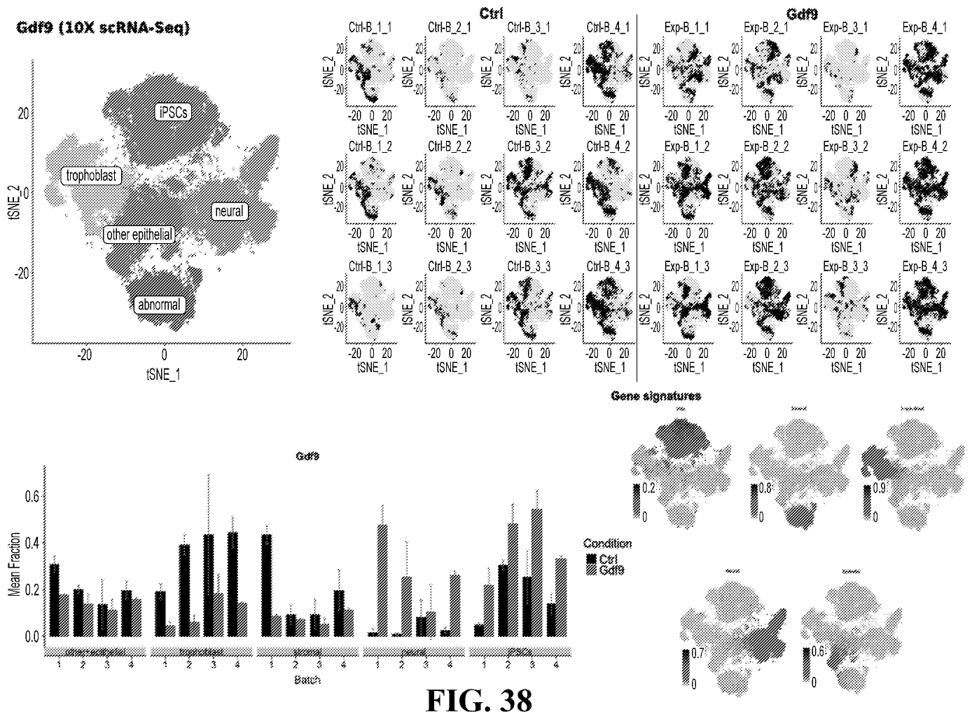

FIG. 38 shows adding GDF9 to the medium resulted in more iPSCs.

Discussion

Understanding the trajectories of cellular differentiation was important for studying development and for regenerative medicine. Large-scale, single-cell profiling had dramatically advanced progress toward this goal. However, the challenge of turning snapshots from single-cell profiling into accurate movies of cellular differentiation had not yet been fully solved. Here, we described two resources for the scientific community: a new analytical approach to reconstructing trajectories, and a massive dataset of 315,000 cells from time courses of classic reprogramming from fibroblasts to iPSCs under two conditions. By applying the approach to the dataset, we shed new light on this well-studied problem, and provide a template for future studies in other systems.

An optimal transport framework to model cell differentiation

Waddington-OT provided an inherently probabilistic approach that described transitions between time points in terms of stochastic couplings, derived from a modified version of the mathematical method of optimal transport. The approach yielded a natural concept of trajectories in terms of ancestor and descendant distributions for any set of cells at a given time point. This allowed us gracefully to recover, for example, branching events (by the emergence of bimodality in the descendant distribution) or shared vs. distinct ancestry between two cell sets (by convergence of the ancestor distributions) (FIGS. 23C-23E). The trajectories can then be used to study differentiation between classes of cells at different times, including creating regulatory models to infer TFs involved in activating specific gene-expression programs. Our model did not impose strict structural constraints a priori on the nature of these processes, allowing for gradual changes over time rather than sharp discrete transitions. Moreover, OT can be applied to even a single pair of time points (if the transition is expected to be sufficiently smooth) and thus can be helpful even for a small experimental scheme. Indeed, we validated Waddington-OT by testing its ability to accurately infer cellular distributions at held-out intermediate time points and by showing that its results are robust across wide variation in parameters.

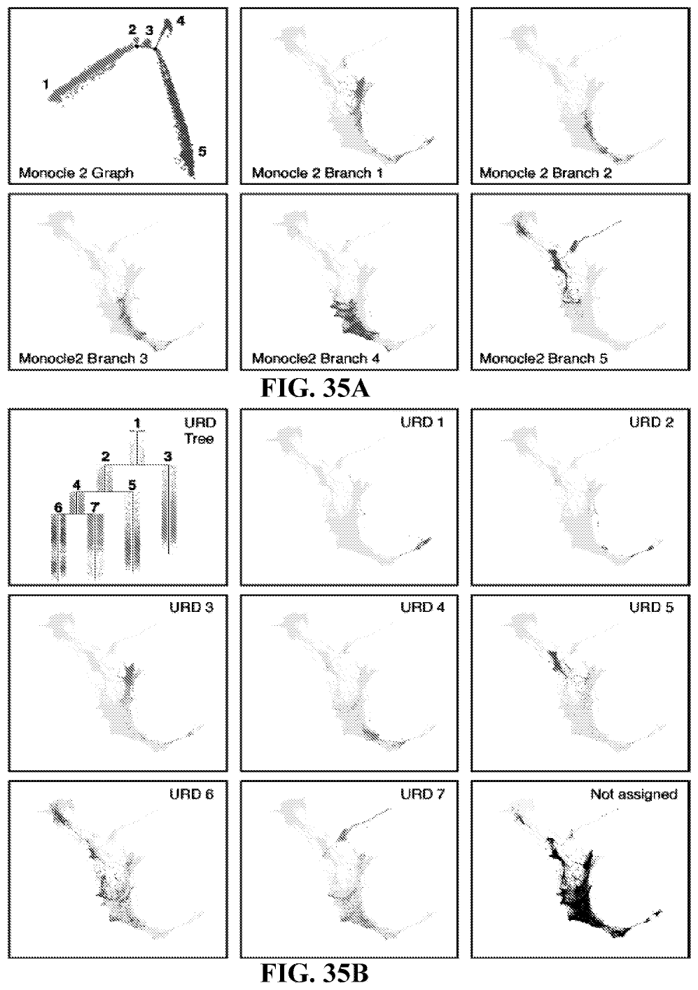

Waddington-OT differred from previous approaches because it (i) did not attempt to force cells onto a simple branching graph, (ii) made explicit use of temporal information, and (iii) allowed for cell growth and death. We also found that Waddington-OT appeared to perform better than several graph-based methods, at least for studying cellular reprogramming from fibroblasts to iPSCs (FIGS. 35A-35B, Methods). Specifically, the widely and successfully used program Monocle2 (Qiu et al., 2017) generated trajectories that a) were inconsistent with known information about time (day 18 stromal cells give rise to essentially all cells after day 0), and b) placed neural and iPS together as one terminal state. The recently developed program URD (Farrell et al., 2018) could avoid the latter problem by finding trajectories to specific cell sets of interest, but a) it generated trajectories which contradicted the gradual MET/Stromal fate specification we saw in our data (in URD, the stromal branch completely diverges at day 0.5), and b) the binary nature of the URD tree could not capture the multifurcation of neural, iPS, trophoblast and epithelial cells from MET.

Tracking cell differentiation trajectories and fates in a diverse reprogramming landscape

Although the reprogramming of fibroblasts to iPSCs had been intensively studied since it was discovered by Yamanaka, our study shedded new light on the process—providing insights that could only be obtained from large-scale single-cell profiles across dense time courses matched with appropriate analytical methods.

First, single-cell profiling with large numbers of cells along a dense time course revealed remarkable and unappreciated diversity in the reprogramming landscape, with large classes of cells having distinct biological programs, related to distinct states and tissues (pluripotency, trophoblasts, neural tissue, epithelium and stroma). In earlier studies based on bulk RNA analysis, we and others had detected expression of individual genes characteristic of various lineages during reprogramming. (Mikkelsen et al., 2008; O'Malley et al., 2013; Parenti et al., 2016). Studying these classes in greater detail, we found a tremendous richness of cells expressing distinct gene-expression programs associated with specific cell types in vivo. Examples included: (i) within iPSC-like cells, programs associated with 2-, 4-, 8-, 16-, and 32-cell stage embryos; (ii) within extra-embryonic-like cells, programs associated with several distinct types of trophoblasts and programs associated with primitive endoderm (at one time point); (iii) within neural-like cells, programs associated with astrocytes, oligodendrocytes, and neurons, as well as specific subprograms associated with excitatory and inhibitory neurons; and (iv) within stromal-like cells, distinct programs associated with a wider range of stromal cells than simply MEFs. Further work will be needed to determine the extent to which these cell types adopt the full identity of natural cell types that they resemble.

This dramatic diversity raised several key questions that Waddington-OT has helped us begin to address, including: (1) What are the differentiation and fate trajectories that span these cell subsets? When do they diverge, from which ancestors, and to which cells do they give rise? (2) What cell intrinsic regulatory mechanisms may drive each fate, especially transcription factors? (3) What might be the role of cells of different types at cross-communicating and supporting across differentiation trajectories and fates in general, and for the iPSC fate in particular?

First, our trajectory and regulatory analysis allowed us to build a model that synthesizes a comprehensive view of the differentiation and fate trajectories in the landscape (FIG. 29D). We highlighted several key fate decisions, in a manner that allowed us to understand their gradual and continuous nature. During the initial phase of reprogramming, cells began to diverge in two alternative directions: toward stromal cells or toward an MET state (FIG. 29D, blue and purple). In the MET direction this divergence was not sharp: although some ancestors exhibited biases in cell fate as early as day 1.5, cells continued to ‘switch’ their fate preference from MET to Stromal up to day 8 (FIGS. 29A-29D, arrows from purple to blue zones). In contrast, the Stromal Region was terminal, and the reverse phenomenon was not seen by our model. Following withdrawal of dox at day 8, the cells in the MET state gave rise to iPSC-, trophoblast-, neural-, and epithelial-like cells. We found no evidence that particular cells had biases towards any of these fates before this point, whereas our analysis clearly distinguished the biases that arise once dox was withdrawn. The ancestors that would lead to iPSCs were distinguished early after withdrawal (day 9), and they passed through a narrow bottleneck towards iPSC. Conversely, other cells in the MET region first assumed an epithelial-like state, with ancestors leading to trophoblasts vs. neural cells (in serum) becoming distinguished a few days later. Within neural cells (in serum) and trophoblast-like cells (in both conditions), there was substantial additional divergence, which we could at times trace to additional divergence between ancestors at later time point. For example, the radial glial population expressing Gdf10 RG at day 13.5 was enriched for ancestors of later emerging neuron-like cells.

Second, by characterizing events that occurred along the trajectory toward any cell class, we identified TFs that might drive subsequent fates (FIG. 29D). Along the path toward pluripotency, we readily rediscovered known TFs, validating our approach, but also identified several new TFs not previously implicated in the process. We tested one such new TF, Obox6, which was associated with a strong bias toward MET early and toward pluripotency late; we found that forced expression of Obox6 increased reprogramming efficiency. Along paths to other fates, we similarly rediscovered TFs known to play a role in differentiation of the corresponding cells in vivo, as well as identified TFs that were expressed in the target cell type but had not been implicated in differentiation per se.

Third, contemporaneous expression of receptor-ligand pairs across cell subsets highlighted potential paracrine interactions between the stromal cells and the iPSC-like, neural-like and trophoblast-like cells, which might play key roles in the initial differentiation and maintenance of these cell types. If many of these potential interactions could be validated by experimental assays, it would suggest that efficient reprogramming requires alternative cell types, or the exogenous replacement of the factors they supply. Additionally, single-cell expression revealed likely regions of genomic aberration; the frequency of such events was significantly higher in our trophoblast and stromal cells, consistent with known biological properties of these cell types.

Prospects for models and studies of differentiation and development

Our method captured several key aspects of cellular differentiation and, importantly, can be extended to capture additional features. First, the framework currently assumed that a cell's trajectory depended only on its current gene-expression levels. As it became possible to perform single-cell profiling simultaneously for gene expression and epigenomic states, one can readily incorporate both types of information. Second, our framework for learning regulatory models assume that trajectories are cell autonomous, but may be extended to incorporate intercellular interactions, such as the potential paracrine signaling postulated here, by using optimal transport for interacting particles (Ambrosio et al., 2008; Santambrogio, 2015) (STAR Methods). Third, various methods are being developed for obtaining lineage information about cells, based on the introduction of barcodes at discrete time points or even continuously (Frieda et al., 2017; McKenna et al., 2016). Barcodes can be used to recognize cells that descend from a recent common ancestor cell, but do not currently directly reveal the full gene-expression state of the ancestral cell. However, they can be incorporated into our optimal-transport framework to improve the inference of ancestral cell states. Finally, our method can be refined to analyze multiple time points simultaneously, rather than just pairs of consecutive time points; this can be particularly useful for situations where the number of cells at different time points varies significantly.

In summary, our findings indicated that the process of reprogramming fibroblasts to iPSCs unleashed a much wider range of developmental programs and subprograms than previously characterized.

### REFERENCES

Aaronson, Y., Livyatan, I., Gokhman, D., and Meshorer, E. (2016). Systematic identification of gene family regulators in mouse and human embryonic stem cells. Nucleic Acids Research 44, 4080-4089.
Daniel et al., (2018). A revised airway epithelial hierarchy includes CFTR-expressing ionocytes. Nature 2018, accepted.
Ambrosio, L., Gigli, N., and Savaré, G. (2008). Gradient flows: in metric spaces and in the space of probability measures (Springer Science & Business Media).
Bastian, M., Heymann, S., Jacomy, M., et al. (2009). Gephi: an open source software for exploring and manipulating networks. Icwsm, 8:361-362.
Bendall, S. C., Davis, K. L., Amir, E.-a.D., Tadmor, M. D., Simonds, E. F., Chen, T. J., Shenfeld, D. K., Nolan, G. P., and Pe'er, D. (2014). Single-cell trajectory detection uncovers progression and regulatory coordination in human B cell development. Cell 157, 714-725.
Beygelzimer, A., Kakadet, S., Langford, J., Arya, S., Mount, D., Li, S., and Li, M. S. (2015). Package FNN.
Boheler, K. R. (2009). Stem cell pluripotency: a cellular trait that depends on transcription factors, chromatin state and a checkpoint deficient cell cycle. Journal of cellular physiology 221, 10-17.
Briggs, J. A., Weinreb, C., Wagner, D. E., Megason, S., Peshkin, L., Kirschner, M. W., and Klein, A. M. (2018). The dynamics of gene expression in vertebrate embryogenesis at single-cell resolution. Science.
Buganim, Y., Faddah, D. A., Cheng, A. W., Itskovich, E., Markoulaki, S., Ganz, K., Klemm, S. L., van Oudenaarden, A., and Jaenisch, R. (2012). Single-cell expression analyses during cellular reprogramming reveal an early stochastic and a late hierarchic phase. Cell 150, 1209-1222.
Cacchiarelli, D., Trapnell, C., Ziller, M. J., Soumillon, M., Cesana, M., Karnik, R., Donaghey, J., Smith, Z. D., Ratanasirintrawoot, S., Zhang, X., Ho Sui, S. J., Wu, Z., Akopian, V., Gifford, C. A., Doench, J., Rinn, J. L., Daley, G. Q., Meissner, A., Lander, E. S., and Mikkelsen, T. (2015). Integrative Analyses of Human Reprogramming Reveal Dynamic Nature of Induced Pluripotency. Cell 162.
Cannoodt, R., Saelens, W., Sichien, D., Tavernier, S., Janssens, S., Guilliams, M., Lambrecht, B. N., De Preter, K., and Saeys, Y. (2016). SCORPIUS improves trajectory inference and identifies novel modules in dendritic cell development. bioRxiv.
Chen, E. Y., Tan, C. M., Kou, Y., Duan, Q., Wang, Z., Meirelles, G. V., Clark, N. R., and Ma'ayan, A. (2013). Enrichr: interactive and collaborative HTML5 gene list enrichment analysis tool. BMC Bioinformatics 14, 128.
Chen, Q., Zhang, M., Li, Y., Xu, D., Wang, Y., Song, A., Zhu, B., Huang, Y., and Zheng, J. C. (2015). CXCR7 Mediates Neural Progenitor Cells Migration to CXCL12 Independent of CXCR4. Stem cells (Dayton, Ohio) 33, 2574-2585.
Chizat, L., Peyre, G., Schmitzer, B., and Vialard, F.-X. (2017). Scaling algorithms for unbalanced transport problems. arXiv preprint arXiv:160705816v2.
Coppé, J.-P., Desprez, P.-Y., Krtolica, A., and Campisi, J. (2010). The senescence-associated secretory phenotype: the dark side of tumor suppression. Annual Review of Pathological Mechanical Disease 5, 99-118.
Cuturi, M. (2013). Sinkhorn distances: Lightspeed computation of optimal transport. Paper presented at: Advances in neural information processing systems.
Elson, G. C., Lelievre, E., Guillet, C., Chevalier, S., Plun-Favreau, H., Froger, J., Suard, I., de Coignac, A. B., Delneste, Y., and Bonnefoy, J.-Y. (2000). CLF associates with CLC to form a functional heteromeric ligand for the CNTF receptor complex. Nature neuroscience 3, 867.
Falco, G., Lee, S. L., Stanghellini, I., Bassey, U. C., Hamatani, T., and Ko, M. S. (2007). Zscan4: a novel gene expressed exclusively in late 2-cell embryos and embryonic stem cells. Developmental biology 307, 539-550.
Farrell, J. A., Wang, Y., Riesenfeld, S. J., Shekhar, K., Regev, A., and Schier, A. F. (2018). Single-cell reconstruction of developmental trajectories during zebrafish embryogenesis. Science.
Fincher, C. T., Wurtzel, O., de Hoog, T., Kravarik, K. M., and Reddien, P. W. (2018). Cell type transcriptome atlas for the planarian <em>Schmidtea mediterranea</em>. Science.
Florio, M., and Huttner, W. B. (2014). Neural progenitors, neurogenesis and the evolution of the neocortex. Development 141, 2182-2194.
Fonseca, E. T.d., Man?anares, A. C. F., Ambr®Æsio, C. E., and Miglino, M. A.1. (2013). Review point on neural stem cells and neurogenic areas of the central nervous system. Open Journal of Animal Sciences Vol. 03No. 03, 6.
Frieda, K. L., Linton, J. M., Hormoz, S., Choi, J., Chow, K.-H. K., Singer, Z. S., Budde, M. W., Elowitz, M. B., and Cai, L. (2017). Synthetic recording and in situ readout of lineage information in single cells. Nature 541, 107.
Froidure, A., Marchal-Duval, E., Ghanem, M., Gerish, L., Jaillet, M., Crestani, B., and Mailleux, A. (2016). Mesenchyme associated transcription factor PRRX1: A key regulator of IPF fibroblast. European Respiratory Journal 48.
Gegenschatz-Schmid, K., Verkauskas, G., Demougin, P., Bilius, V., Dasevicius, D., Stadler, M. B., and Hadziselimovic, F. (2017). DMRTC2, PAX7, BRACHYURY/T and TERT Are Implicated in Male Germ Cell Development Following Curative Hormone Treatment for Cryptorchidism-Induced Infertility. Genes 8, 267.
Goolam, M., Scialdone, A., Graham, S. J. L., Macaulay, I. C., Jedrusik, A., Hupalowska, A., Voet, T., Marioni, J. C., and Zernicka-Goetz, M. (2016). Heterogeneity in Oct4 and Sox2 Targets Biases Cell Fate in 4-Cell Mouse Embryos. Cell 165, 61-74.
Gouti, M., Briscoe, J., and Gavalas, A. (2011). Anterior Hox genes interact with components of the neural crest specification network to induce neural crest fates. Stem cells (Dayton, Ohio) 29, 858-870.
Haghverdi, L., Buettner, F., and Theis, F. J. (2015). Diffusion maps for high-dimensional single-cell analysis of differentiation data. Bioinformatics 31, 2989-2998.
Haghverdi, L., Buettner, M., Wolf, F. A., Buettner, F., and Theis, F. J. (2016). Diffusion pseudonyme robustly reconstructs lineage branching. bioRxiv, 041384.
Han, X., Wang, R., Zhou, Y., Fei, L., Sun, H., Lai, S., Saadatpour, A., Zhou, Z., Chen, H., Ye, F., et al. (2018). Mapping the Mouse Cell Atlas by Microwell-Seq. Cell 172, 1091-1107.e1017.
Hayashi, Y., Hsiao, E. C., Sami, S., Lancero, M., Schlieve, C. R., Nguyen, T., Yano, K., Nagahashi, A., Ikeya, M., Matsumoto, Y., et al. (2016). BMP-SMAD-ID promotes reprogramming to pluripotency by inhibiting p16/INK4A-dependent senescence. Proceedings of the National Academy of Sciences of the United States of America 113, 13057-13062.
Hou, P., Li, Y., Zhang, X., Liu, C., Guan, J., Li, H., Zhao, T., Ye, J., Yang, W., Liu, K., et al. (2013). Pluripotent Stem Cells Induced from Mouse Somatic Cells by Small-Molecule Compounds. Science 341, 651-654.
Hu, G., Kim, J., Xu, Q., Leng, Y., Orkin, S. H., and Elledge, S. J. (2009). A genome-wide RNAi screen identifies a new transcriptional module required for self-renewal. Genes & development 23, 837-848.
Hussein, S. M., Puri, M. C., Tonge, P. D., Benevento, M., Corso, A. J., Clancy, J. L., Mosbergen, R., Li, M., Lee, D.-S., and Cloonan, N. (2014). Genome-wide characterization of the routes to pluripotency. Nature 516, 198.
Jacomy, M., Venturini, T., Heymann, S., and Bastian, M. (2014). ForceAtlas2, a continuous graph layout algorithm for handy network visualization designed for the Gephi software. PloS one 9, e98679.
Jeon, H., Waku, T., Azami, T., Khoa le, T. P., Yanagisawa, J., Takahashi, S., and Ema, M. (2016). Comprehensive Identification of Kruppel-Like Factor Family Members Contributing to the Self-Renewal of Mouse Embryonic Stem Cells and Cellular Reprogramming. PloS one 11, e0150715.
Jukkola, T., Lahti, L., Naserke, T., Wurst, W., and Partanen, J. (2006). FGF regulated gene-expression and neuronal differentiation in the developing midbrain-hindbrain region. Developmental biology 297, 141-157.
Kan, L., Israsena, N., Zhang, Z., Hu, M., Zhao, L. R., Jalali, A., Sahni, V., and Kessler, J. A. (2004). Sox1 acts through multiple independent pathways to promote neurogenesis. Developmental biology 269, 580-594.
Kantorovitch, L. (1958). On the Translocation of Masses. Management Science 5, 1-4.
Kester, L., and van Oudenaarden, A. (2018). Single-Cell Transcriptomics Meets Lineage Tracing. Cell Stem Cell.
Kidder, B. L., and Palmer, S. (2010). Examination of transcriptional networks reveals an important role for TCFAP2C, SMARCA4, and EOMES in trophoblast stem cell maintenance. Genome Res 20, 458-472.
Kim, D. H., Marinov, G. K., Pepke, S., Singer, Z. S., He, P., Williams, B., Schroth, G. P., Elowitz, M. B., and Wold, B. J. (2015). Single-cell transcriptome analysis reveals dynamic changes in lncRNA expression during reprogramming. Cell stem cell 16, 88-101.
Klein, A. M., Mazutis, L., Akartuna, I., Tallapragada, N., Veres, A., Li, V., Peshkin, L., Weitz, D. A., and Kirschner, M. W. (2015). Droplet barcoding for single-cell transcriptomics applied to embryonic stem cells. Cell 161, 1187-1201.
Kolodziejczyk, Aleksandra A., Kim, Jong K., Tsang, Jason C., Ilicic, T., Henriksson, J., Natarajan, Kedar N., Tuck, Alex C., Gao, X., Btihler, M., Liu, P., et al. (2015). Single Cell RNA-Sequencing of Pluripotent States Unlocks Modular Transcriptional Variation. Cell Stem Cell 17, 471-485.
Kumar, R. M., Cahan, P., Shalek, A. K., Satija, R., Jay DaleyKeyser, A., Li, H., Zhang, J., Pardee, K., Gennert, D., Trombetta, J. J., et al. (2014). Deconstructing transcriptional heterogeneity in pluripotent stem cells. Nature 516, 56.
Latos, P. A., and Hemberger, M. (2016). From the stem of the placental tree: trophoblast stem cells and their progeny. Development 143, 3650-3660.
Lattin, J. E., Schroder, K., Su, A. I., Walker, J. R., Zhang, J., Wiltshire, T., Saijo, K., Glass, C. K., Hume, D. A., Kellie, S., et al. (2008). Expression analysis of G Protein-Coupled Receptors in mouse macrophages. Immunome research 4, 5.
Lazarov, O., Mattson, M. P., Peterson, D. A., Pimplikar, S. W., and van Praag, H. (2010). When neurogenesis encounters aging and disease. Trends in neurosciences 33, 569-579.
Le'onard, C. (2014). A survey of the schrödinger problem and some of its connections with optimal transport. Discrete and Continuous Dynamical Systems—Series A (DCDS-A), 34(4):1533-1574.
Li, R., Liang, J., Nᵢ, S., Zhou, T., Qing, X., Li, H., He, W., Chen, J., Li, F., Zhuang, Q., et al. (2010). A mesenchymal-to-epithelial transition initiates and is required for the nuclear reprogramming of mouse fibroblasts. Cell Stem Cell 7, 51-63.
Li, W.-Z., Wang, Z.-W., Chen, L.-L., Xue, H.-N., Chen, X., Guo, Z.-K., and Zhang, Y. (2015). Hesx1 enhances pluripotency by working downstream of multiple pluripotency-associated signaling pathways. Biochemical and Biophysical Research Communications 464, 936-942.
Liang, H., Zhang, Q., Lu, J., Yang, G., Tian, N., Wang, X., Tan, Y., and Tan, D. (2016). MSX2 Induces Trophoblast Invasion in Human Placenta. PloS one 11, e0153656.
Lim, L. S., Loh, Y. H., Zhang, W., Li, Y., Chen, X., Wang, Y., Bakre, M., Ng, H. H., and Stanton, L. W. (2007). Zic3 is required for maintenance of pluripotency in embryonic stem cells. Molecular biology of the cell 18, 1348-1358.
Lin, J., Khan, M., Zapiec, B., and Mombaerts, P. (2016). Efficient derivation of extraembryonic endoderm stem cell lines from mouse postimplantation embryos. Scientific reports 6, 39457.
Liu, J., Han, Q., Peng, T., Peng, M., Wei, B., Li, D., Wang, X., Yu, S., Yang, J., Cao, S., et al. (2015). The oncogene c-Jun impedes somatic cell reprogramming. Nature cell biology 17, 856-867.
Liu, L. L., Brumbaugh, J., Bar-Nur, O., Smith, Z., Stadtfeld, M., Meissner, A., Hochedlinger, K., and Michor, F. (2016). Probabilistic Modeling of Reprogramming to Induced Pluripotent Stem Cells. Cell reports 17, 3395-3406.
Ma, G. T., Roth, M. E., Groskopf, J. C., Tsai, F. Y., Orkin, S. H., Grosveld, F., Engel, J. D., and Linzer, D. I. (1997). GATA-2 and GATA-3 regulate trophoblast-specific gene expression in vivo. Development 124, 907-914.
Macfarlan, T. S., Gifford, W. D., Driscoll, S., Lettieri, K., Rowe, H. M., Bonanomi, D., Firth, A., Singer, O., Trono, D., and Pfaff, S. L. (2012). Embryonic stem cell potency fluctuates with endogenous retrovirus activity. Nature 487, 57-63.
Macosko, E. Z., Basu, A., Satija, R., Nemesh, J., Shekhar, K., Goldman, M., Tirosh, I., Bialas, A. R., Kamitaki, N., and Martersteck, E. M. (2015). Highly parallel genome-wide expression profiling of individual cells using nanoliter droplets. Cell 161, 1202-1214.
Marco, E., Karp, R. L., Guo, G., Robson, P., Hart, A. H., Trippa, L., and Yuan, G. C. (2014). Bifurcation analysis of single-cell gene expression data reveals epigenetic landscape. Proceedings of the National Academy of Sciences of the United States of America 111, E5643-5650.
Matsumoto, H., and Kiryu, H. (2016). SCOUP: a probabilistic model based on the Ornstein-Uhlenbeck process to analyze single-cell expression data during differentiation. BMC Bioinformatics 17, 232.
McKenna, A., Findlay, G. M., Gagnon, J. A., Horwitz, M. S., Schier, A. F., and Shendure, J. (2016). Whole-organism lineage tracing by combinatorial and cumulative genome editing. Science 353, aaf7907.
Mertins, P., Przybylski, D., Yosef, N., Qiao, J., Clauser, K., Raychowdhury, R., Eisenhaure, T. M., Maritzen, T., Haucke, V., Satoh, T., et al. (2017). An Integrative Framework Reveals Signaling-to-Transcription Events in Toll-like Receptor Signaling. Cell reports 19, 2853-2866.
Messina, G., Biressi, S., Monteverde, S., Magli, A., Cassano, M., Perani, L., Roncaglia, E., Tagliafico, E., Starnes, L., Campbell, C. E., et al. (2010). Nfix regulates fetal-specific transcription in developing skeletal muscle. Cell 140, 554-566.
Mikkelsen, T. S., Hanna, J., Zhang, X., Ku, M., Wernig, M., Schorderet, P., Bernstein, B. E., Jaenisch, R., Lander, E. S., and Meissner, A. (2008). Dissecting direct reprogramming through integrative genomic analysis. Nature 454, 49.
Ming, G. L., and Song, H. (2011). Adult neurogenesis in the mammalian brain: significant answers and significant questions. Neuron 70, 687-702.
Mosteiro, L., Pantoja, C., Alcazar, N., Mari6n, R. M., Chondronasiou, D., Rovira, M., Fernandez-Marcos, P. J., Mufioz-Martin, M., Blanco-Aparicio, C., and Pastor, J. (2016). Tissue damage and senescence provide critical signals for cellular reprogramming in vivo. Science 354, aaf4445.
Nakashima, K., Wiese, S., Yanagisawa, M., Arakawa, H., Kimura, N., Hisatsune, T., Yoshida, K., Kishimoto, T., Sendtner, M., and Taga, T. (1999). Developmental requirement of gpl30 signaling in neuronal survival and astrocyte differentiation. The Journal of neuroscience: the official journal of the Society for Neuroscience 19, 5429-5434.
Nelson, A. C., Mould, A. W., Bikoff, E. K., and Robertson, E. J. (2016). Single-cell RNA-seq reveals cell type-specific transcriptional signatures at the maternal-foetal interface during pregnancy. Nat Commun 7, 11414.
O'Malley, J., Skylaki, S., Iwabuchi, K. A., Chantzoura, E., Ruetz, T., Johnsson, A., Tomlinson, S. R., Linnarsson, S., and Kaji, K. (2013). High resolution analysis with novel cell-surface markers identifies routes to iPS cells. Nature 499, 88.
Ocana, O. H., Corcoles, R., Fabra, A., Moreno-Bueno, G., Acloque, H., Vega, S., Barrallo-Gimeno, A., Cano, A., and Nieto, M. A. (2012). Metastatic colonization requires the repression of the epithelial-mesenchymal transition inducer Prrx1. Cancer cell 22, 709-724.
Parast, M. M., Yu, H., Ciric, A., Salata, M. W., Davis, V., and Milstone, D. S. (2009). PPARgamma regulates trophoblast proliferation and promotes labyrinthine trilineage differentiation. PloS one 4, e8055.
Parenti, A., Halbisen, M. A., Wang, K., Latham, K., and Ralston, A. (2016). OSKM induce extraembryonic endoderm stem cells in parallel to induced pluripotent stem cells. Stem cell reports 6, 447-455.
Park, M., Lee, Y., Jang, H., Lee, O. H., Park, S. W., Kim, J. H., Hong, K., Song, H., Park, S. P., Park, Y. Y., et al. (2016). SOHLH2 is essential for synaptonemal complex formation during spermatogenesis in early postnatal mouse testes. Scientific reports 6, 20980.
Pasque, V., Tchieu, J., Karnik, R., Uyeda, M., Dimashkie, A. S., Case, D., Papp, B., Bonora, G., Patel, S., and Ho, R. (2014). X chromosome reactivation dynamics reveal stages of reprogramming to pluripotency. Cell 159, 1681-1697.
Patel, A. P., Tirosh, I., Trombetta, J. J., Shalek, A. K., Gillespie, S. M., Wakimoto, H., Cahill, D. P., Nahed, B. V., Curry, W. T., Martuza, R. L., et al. (2014). Single-cell RNA-seq highlights intratumoral heterogeneity in primary glioblastoma. Science (New York, N. Y.) 344, 1396-1401.
Pei, J., and Grishin, N. V. (2012). Unexpected diversity in Shisa-like proteins suggests the importance of their roles as transmembrane adaptors. Cellular signalling 24, 758-769.
Plass, M., Solana, J., Wolf, F. A., Ayoub, S., Misios, A., Glaiar, P., Obermayer, B., Theis, F. J., Kocks, C., and Rajewsky, N. (2018). Cell type atlas and lineage tree of a whole complex animal by single-cell transcriptomics. Science.
Polo, J. M., Anderssen, E., Walsh, R. M., Schwarz, B. A., Nefzger, C. M., Lim, S. M., Borkent, M., Apostolou, E., Alaei, S., and Cloutier, J. (2012). A molecular roadmap of reprogramming somatic cells into iPS cells. Cell 151, 1617-1632.
Porpiglia, E., Samusik, N., Van Ho, A. T., Cosgrove, B. D., Mai, T., Davis, K. L., Jager, A., Nolan, G. P., Bendall, S. C., Fantl, W. J., et al. (2017). High-resolution myogenic lineage mapping by single-cell mass cytometry. Nature Cell Biol., 19:558-567.
Qiu, X., Mao, Q., Tang, Y., Wang, L., Chawla, R., Pliner, H., and Trapnell, C. (2017). Reversed graph embedding resolves complex single-cell developmental trajectories. bioRxiv, 110668.
Rajkovic, A., Yan, C., Yan, W., Klysik, M., and Matzuk, M. M. (2002). Obox, a Family of Homeobox Genes Preferentially Expressed in Germ Cells. Genomics 79, 711-717.
Ralston, A., Cox, B. J., Nishioka, N., Sasaki, H., Chea, E., Rugg-Gunn, P., Guo, G., Robson, P., Draper, J. S., and Rossant, J. (2010). Gata3 regulates trophoblast development downstream of Tead4 and in parallel to Cdx2. Development 137, 395-403.
Ramsköld, D., Luo, S., Wang, Y.-C., Li, R., Deng, Q., Faridani, O. R., Daniels, G. A., Khrebtukova, I., Loring, J. F., Laurent, L. C., et al. (2012). Full-Length mRNA-Seq from single cell levels of RNA and individual circulating tumor cells. Nature biotechnology 30, 777-782.
Rashid, S., Kotton, D. N., and Bar-Joseph, Z. (2017). TASIC: determining branching models from time series single cell data. Bioinformatics 33, 2504-2512.
Richard Jordan, D. K. and Otto, F. (1998). The variational formulation of the fokker. SIAM J. Math. Anal., 29(1):1-17.
Rostom, R., Svensson, V., Teichmann, S., and Kar, G. (2017). Computational approaches for interpreting scRNA-seq data. FEBS letters.
Sakakibara, S., Nakamura, Y., Satoh, H., and Okano, H. (2001). Rna-binding protein Musashi2: developmentally regulated expression in neural precursor cells and subpopulations of neurons in mammalian CNS. The Journal of neuroscience: the official journal of the Society for Neuroscience 21, 8091-8107.
Samusik, N., Good, Z., Spitzer, M. H., Davis, K. L., and Nolan, G. P. (2016). Automated mapping of phenotype space with single-cell data. Nature methods, 13:493-496.
Sansom, S. N., Griffiths, D. S., Faedo, A., Kleinjan, D. J., Ruan, Y., Smith, J., van Heyningen, V., Rubenstein, J. L., and Livesey, F. J. (2009). The level of the transcription factor Pax6 is essential for controlling the balance between neural stem cell self-renewal and neurogenesis. PLoS genetics 5, e1000511.
Santambrogio, F. (2015). Optimal transport for applied mathematicians. Birkäuser, NY, 99-102.
Satija, R., Farrell, J. A., Gennert, D., Schier, A. F., and Regev, A. (2015). Spatial reconstruction of single-cell gene expression data. Nature Biotechnology 33, 495.
Scott, I. C., Anson-Cartwright, L., Riley, P., Reda, D., and Cross, J. C. (2000). The HAND1 basic helix-loop-helix transcription factor regulates trophoblast differentiation via multiple mechanisms. Molecular and cellular biology 20, 530-541.
Setty, M., Tadmor, M. D., Reich-Zeliger, S., Angel, O., Salame, T. M., Kathail, P., Choi, K., Bendall, S., Friedman, N., and Pe'er, D. (2016). Wishbone identifies bifurcating developmental trajectories from single-cell data. Nature biotechnology 34, 637-645.
Shalek, A. K., Satija, R., Adiconis, X., Gertner, R. S., Gaublomme, J. T., Raychowdhury, R., Schwartz, S., Yosef, N., Malboeuf, C., Lu, D., et al. (2013). Single-cell transcriptomics reveals bimodality in expression and splicing in immune cells. Nature 498, 236.
Shi, W., Wang, H., Pan, G., Geng, Y., Guo, Y., and Pei, D. (2006). Regulation of the pluripotency marker Rex-1 by Nanog and Sox2. J Biol Chem 281, 23319-23325.
Shu, J., Wu, C., Wu, Y., Li, Z., Shao, S., Zhao, W., Tang, X., Yang, H., Shen, L., Zuo, X., et al. (2013). Induction of pluripotency in mouse somatic cells with lineage specifiers. Cell 153, 963-975.
Simmons, D. G., and Cross, J. C. (2005). Determinants of trophoblast lineage and cell subtype specification in the mouse placenta. Developmental biology 284, 12-24.
Simmons, D. G., Natale, D. R., Begay, V., Hughes, M., Leutz, A., and Cross, J. C. (2008). Early patterning of the chorion leads to the trilaminar trophoblast cell structure in the placental labyrinth. Development 135, 2083-2091.
Stadtfeld, M., Maherali, N., Borkent, M., and Hochedlinger, K. (2010). A reprogrammable mouse strain from gene-targeted embryonic stem cells. Nature methods 7, 53-55.
Street, K., Risso, D., Fletcher, R. B., Das, D., Ngai, J., Yosef, N., Purdom, E., and Dudoit, S. (2017). Slingshot: Cell lineage and pseudotime inference for single-cell transcriptomics. bioRxiv.
Takahashi, K., and Yamanaka, S. (2006). Induction of pluripotent stem cells from mouse embryonic and adult fibroblast cultures by defined factors. cell 126, 663-676.
Takahashi, K., and Yamanaka, S. (2016). A decade of transcription factor-mediated reprogramming to pluripotency. Nature Reviews Molecular Cell Biology 17, 183.
Takaishi, M., Tarutani, M., Takeda, J., and Sano, S. (2016). Mesenchymal to Epithelial Transition Induced by Reprogramming Factors Attenuates the Malignancy of Cancer Cells. PloS one 11, e0156904.
Tanay, A., and Regev, A. (2017). Scaling single-cell genomics from phenomenology to mechanism. Nature 541, 331-338.
Tang, F., Barbacioru, C., Wang, Y., Nordman, E., Lee, C., Xu, N., Wang, X., Bodeau, J., Tuch, B. B., Siddiqui, A., et al. (2009). mRNA-Seq whole-transcriptome analysis of a single cell. Nature Methods 6, 377.
Tasic, B., Menon, V., Nguyen, T. N., Kim, T. K., Jarsky, T., Yao, Z., Levi, B., Gray, L. T., Sorensen, S. A., Dolbeare, T., et al. (2016). Adult mouse cortical cell taxonomy revealed by single cell transcriptomics. Nat Neurosci 19, 335-346.
Tirosh, I., Venteicher, A. S., Hebert, C., Escalante, L. E., Patel, A. P., Yizhak, K., Fisher, J. M., Rodman, C., Mount, C., and Filbin, M. G. (2016). Single-cell RNA-seq supports a developmental hierarchy in human oligodendroglioma. Nature 539, 309-313.
Tonge, P. D., Corso, A. J., Monetti, C., Hussein, S. M., Puri, M. C., Michael, I. P., Li, M., Lee, D.-S., Mar, J. C., and Cloonan, N. (2014). Divergent reprogramming routes lead to alternative stem-cell states. Nature 516, 192-197.
Trapnell, C., Cacchiarelli, D., Grimsby, J., Pokharel, P., Li, S., Morse, M., Lennon, N. J., Livak, K. J., Mikkelsen, T. S., and Rinn, J. L. (2014). The dynamics and regulators of cell fate decisions are revealed by pseudotemporal ordering of single cells. Nature biotechnology 32, 381-386.
Ueno, M., Lee, L. K., Chhabra, A., Kim, Y. J., Sasidharan, R., Van Handel, B., Wang, Y., Kamata, M., Kamran, P., Sereti, K.-I., et al. (2013). c-Met-dependent multipotent labyrinth trophoblast progenitors establish placental exchange interface. Developmental cell 27, 373-386.
Vandercappellen, J., Van Damme, J., and Struyf, S. (2008). The role of CXC chemokines and their receptors in cancer. Cancer letters 267, 226-244.
Villani, C. (2008). Optimal transport: old and new, Vol 338 (Springer Science & Business Media).
Waddington, C. H. (1936). How animals develop (New York).
Waddington, C. H. (1957). The strategy of the genes; a discussion of some aspects of theoretical biology (London, Allen & Unwin [1957]).
Wagner, A., Regev, A., and Yosef, N. (2016). Revealing the vectors of cellular identity with single-cell genomics. Nat Biotech 34, 1145-1160.
Wagner, D. E., Weinreb, C., Collins, Z. M., Briggs, J. A., Megason, S. G., and Klein, A. M. (2018). Single-cell mapping of gene expression landscapes and lineage in the zebrafish embryo. Science.
Watanabe, Y., Stanchina, L., Lecerf, L., Gacem, N., Conidi, A., Baral, V., Pingault, V., Huylebroeck, D., and Bondurand, N. (2017). Differentiation of Mouse Enteric Nervous System Progenitor Cells Is Controlled by Endothelin 3 and Requires Regulation of Ednrb by SOX10 and ZEB2. Gastroenterology 152, 1139-1150.e1134.
Weinreb, C., Wolock, S., and Klein, A. (2016). SPRING: a kinetic interface for visualizing high dimensional single-cell expression data. bioRxiv.
Weinreb, C., Wolock, S., Tusi, B. K., Socolovsky, M., and Klein, A. M. (2017). Fundamental limits on dynamic inference from single cell snapshots. bioRxiv.
Welch, J. D., Hartemink, A. J., and Prins, J. F. (2016). SLICER: inferring branched, nonlinear cellular trajectories from single cell RNA-seq data. Genome Biology 17, 106.
Whiteman, E. L., Fan, S., Harder, J. L., Walton, K. D., Liu, C. J., Soofi, A., Fogg, V. C., Hershenson, M. B., Dressler, G. R., Deutsch, G. H., et al. (2014). Crumbs3 is essential for proper epithelial development and viability. Molecular and cellular biology 34, 43-56.
Wu, D., Hong, H., Huang, X., Huang, L., He, Z., Fang, Q., and Luo, Y. (2016). CXCR2 is decreased in preeclamptic placentas and promotes human trophoblast invasion through the Akt signaling pathway. Placenta 43, 17-25.
Wu, L., Wu, Y., Peng, B., Hou, Z., Dong, Y., Chen, K., Guo, M., Li, H., Chen, X., Kou, X., et al. (2017). Oocyte-Specific Homeobox 1, Oboxl, Facilitates Reprogramming by Promoting Mesenchymal-to-Epithelial Transition and Mitigating Cell Hyperproliferation. Stem Cell Reports 9, 1692-1705.
Wu, X., Oatley, J. M., Oatley, M. J., Kaucher, A. V., Avarbock, M. R., and Brinster, R. L. (2010). The POU domain transcription factor POU3F1 is an important intrinsic regulator of GDNF-induced survival and self-renewal of mouse spermatogonial stem cells. Biology of reproduction 82, 1103-1111.
Yamamizu, K., Sharov, A. A., Piao, Y., Amano, M., Yu, H., Nishiyama, A., Dudekula, D. B., Schlessinger, D., and Ko, M. S. (2016). Generation and gene expression profiling of 48 transcription-factor-inducible mouse embryonic stem cell lines. Scientific reports 6, 25667.
Ying, Q.-L., Wray, J., Nichols, J., Batlle-Morera, L., Doble, B., Woodgett, J., Cohen, P., and Smith, A. (2008). The ground state of embryonic stem cell self-renewal. Nature 453, 519.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.
Yun, C., Mendelson, J., Blake, T., Mishra, L., and Mishra, B. (2008). TGF-beta signaling in neuronal stem cells. Disease markers 24, 251-255.
Zhao, T., Fu, Y., Zhu, J., Liu, Y., Zhang, Q., Yi, Z., Chen, S., Jiao, Z., Xu, X., Xu, J., Duo, S., Bai, Y., Tang, C., Li, C., and Deng, H. (2018). Single-Cell RNA-Seq Reveals Dynamic Early Embryonic-like Programs during Chemical Reprogramming. Cell Stem Cell 23, 1-15.
Zunder, E. R., Lujan, E., Goltsev, Y., Wernig, M., and Nolan, G. P. (2015). A continuous molecular roadmap to iPSC reprogramming through progression analysis of single-cell mass cytometry. Cell Stem Cell 16, 323-337.
Zwiessele, M., and Lawrence, N. D. (2016). Topslam: Waddington Landscape Recovery for Single Cell Experiments. bioRxiv.

Key Resources

Key resources used in this study are shown below.

| | | |
|---|---|---|
| REAGENTS or RESOURCE | SOURCE | IDENTIFIER |

Recombinant DNA

| | | |
|---|---|---|
| FUW Tet-On vector | Addgene | #20323 |
| Zfp42 cDNA | Origene | MG203929 |
| Obox6 cDNA | Origene | MR215428 |

Chemicals, Peptides, and Recombinant Proteins

| | | |
|---|---|---|
| leukemia inhibitory factor (LIF) | Millipore | ESG1107 |
| PD0325901 | Sigma | PZ0162-25MG |
| CHIR99021 | Sigma | PZ0162-25MG |

Critical Commercial Kits

| | | |
|---|---|---|
| Chromium ™ Single Cell 3′ Reagent | 10X genomics | PN-120230, PN-120231, |
| Kits v1 | | PN-120232 |
| Chromium ™ Single Cell 3′ Reagent | 10X genomics | PN-120237 |
| Kits v2 | | |
| Fugene HD reagent | Promega | E2311 |

Cloning Reagents

| | | |
|---|---|---|
| Gibson Assembly | NEB | E2611S |
| Sequence-Based Reagents | | |

Deposited Data

| | | |
|---|---|---|
| Single cell RNA-seq raw data | NCBI Gene Expression | GSE106340 |
| (pilot study) | Omnibus | |
| Single cell RNA-seq raw data | NCBI Gene Expression | GSE115943 |
| | Omnibus | |

Experimental Models: Organisms/Strains

| | | |
|---|---|---|
| OKSM secondary MEFs | Konrad Hochedlinger lab | OKSM × B6.Cg- |
| | | Gt(ROSA)26Sor^{tm1(rtTA}*^{M2)Jae}/J × |
| | | B6; 129S4-Pou5fl^{tm2Jae}/J |
| Primary MEFs | Rudolf Jaenisch lab | B6.Cg- |
| | | Gt(ROSA)26Sor^{tm1(rtTA}*^{M2)Jae}/J × |
| | | B6; 129S4-Pou5fl^{tm2Jae}/J |

Software and Algorithms

| | | |
|---|---|---|
| Waddington-OT | This paper | https://github.com/broadinstitute/wot |
| Scaling algorithm for unbalanced | (Chizat et al., 2016) | |
| transport | | |
| CellRanger | 10X genomics | v2.0.0 |
| ForceAtlas2 | Gephi | v0.9.2 |
| Seurat | | v2.1.0 |
| Scanpy | | v0.2.8 |
| Monocle2 | (Qiu et al. 2017) | v2.8.0 |
| URD | (Farrell et al 2018) | v1.0 |

Method Details

I. Modeling Developmental Processes with Optimal Transport

We developed a method to model development based on Optimal Transport. Section 1 reviews the concept of gene expression space and introduces our probabilistic framework for time series of expression profiles. Section 2 introduces our key modeling assumption to infer temporal couplings over short time scales. Section 3 shows how we can compute an optimal coupling between adjacent time points by solving a convex optimization problem, and how we can leverage an assumption of Markovity to compose adjacent time points and estimate temporal couplings over longer intervals. Section 4 describes how to interpret transport maps. Specifically, Section 4.1 shows how to compute ancestors and descendants of cells, Section 4.2 describes an interesting physical interpretation of entropy-regularization, and Section 4.3 shows how we learn gene regulatory networks to summarize the trajectories.

1. Developmental Processes in Gene Expression Space

A collection of mRNA levels for a single cell is called an expression profile and is often represented mathematically by a vector in gene expression space. This is a vector space that has dimension equal to the number of genes, with the value of the ith coordinate of an expression profile vector representing the number of copies of mRNA for the ith gene. Note that real cells only occupy an integer lattice in gene expression space (because the number of copies of mRNA is an integer), but we pretended that cells can move continuously through a real-valued G dimensional vector space.

As an individual cell changes the genes it expresses over time, it moves in gene expression space and describes a trajectory. As a population of cells develops and grows, a distribution on gene expression space evolves over time. When a single cell from such a population is measured with single cell RNA sequencing, we obtained a noisy estimate of the number of molecules of mRNA for each gene. We represented the measured expression profile of this single cell as a sample from a probability distribution on gene expression space. This sampling captured both (a) the randomness in the single-cell RNA sequencing measurement process (due to subsampling reads, technical issues, etc.) and (b) the random selection of a cell from the population. We treated this probability distribution as nonparametric in the sense that it was not specified by any finite list of parameters.

In the remainder of this section we introduced a precise mathematical notion for a developmental process as a generalization of a stochastic process. Our primary goal was to infer the ancestors and descendants of subpopulations evolving according to an unknown developmental process. This information was encoded in the temporal coupling of the process, which is lost because we kill the cells when we perform scRNA-Seq. We claimed it was possible to recover the temporal coupling over short time scales provided that cells don't change too much. Therefore we could make inferences about which cells go where. We showed in the remainder of this section how to do this with optimal transport.

1.1 a Mathematical Model of Developmental Processes

We began by formally defining a precise notion of the developmental trajectory of an individual cell and its descendants. Intuitively, it was a continuous path in gene expression space that bifurcated with every cell division. Formally, we defined it as follows:

Definition 1 (single-cell developmental trajectory). Consider a cell x(0)∈ [symbol] ^{G}: Let k(t)≥0 specify the number of descendants at time t, where k(0)=1. A single-cell development trajectory is a continuous function

$$x : [ 0 , T ) \to \underbrace{{ℝ}^{G} \times {ℝ}^{G} \times … \times {ℝ}^{G}}_{k ( t ) \text{?}} . \\ \text{?} \text{indicates text missing or illegible when filed}$$

This means that x(t) is a k(t)-tuple of cells, each represented by a vector in [symbol] ^{G}:

x(t)=(x₁(t), . . . ,xₖ₍ₜ₎(t)).

We referred to the cells x₁(t), . . . , xₖ₍ₜ₎(t) as the descendants of x(0).

Note that we could not directly measure the temporal dynamics of an individual cell because scRNA-Seq was a destructive measurement process: scRNA-Seq lysed cells so it was possible to measure the expression profile of a cell at a single point in time. As a result, it was not possible to directly measure the descendants of that cell, and the full trajectory was unobservable. However, one can learn something about the probable trajectories of individual cells by measuring snapshots from an evolving population.

Published methods typically represent the aggregate trajectory of a population of cells by means of a graph structure. While this recapitulates the branching path traveled by the descendants of an individual cell, it may over-simplify the stochastic nature of developmental processes. Individual cells have the potential to travel through different paths, but any given cell travels one and only one such path. Our goal was to assign a likelihood to the set of possible paths, which in general were not finite and therefore cannot be a represented by a graph.

We defined a developmental process to be a time-varying probability distribution on gene expression space. One simple example of a distribution of cells is that we can represent a set of cells

x₁, . . . , xₙ by the distribution

$$ℙ = \frac{1}{n} {\sum }_{i = 1}^{n} {δ}_{\text{?}}$$

$$\text{?} \text{indicates text missing or illegible when filed}$$

Similarly, we could represent a set of single-cell trajectories xi(t), . . . , xₙ(t) with a distribution over trajectories. This was a special case of a developmental process, which we defined as follows:

Definition 2 (developmental process). A developmental process Pₜ is a time-varying distribution (i.e. stochastic process) on gene expression space.

Recall that a stochastic process was determined by its temporal dependence structure. This was specified by the coupling (i.e. joint distribution) between random variables at different time points. Given that a cell had a particular expression profile y at time t₂, where did it come from at time t₁? This was the information lost by not tracking individual cells over time.

Definition 3 (temporal coupling). Let Pₜ be a developmental process and consider two time points s<t. Let Xt˜Pₜ denote the expression profile of a random cell at time t and let Xₛdenote the expression profile of the cell of origin at times.

The temporal coupling γ_{s,t} is defined as the law of the joint distribution:

γ_{s,t}= [symbol] (Xₛ,Xₜ).

Equivalently,

∫_{x∈A}∫_{y∈B}γ_{s,t}(x,y)dxdy=Pr{Xₛ∈A,Xₜ∈B}

for any sets A, B⊂ [symbol] ^{G}.

The temporal coupling γ_{s,t} was not technically a coupling of Pₛ and Pₜ in the standard sense because it does not necessarily have marginals Pₛ and Pₜ:

∫γ_{s,t}(x,y)dx= [symbol] ₜ(y), but ∫γ_{s,t}(x,y)dy≠ [symbol] ₛ(x).

Biologically, this was the case when cells grow at different rates. Then proliferative cells from the earlier time point were over-represented when we look for the origin of cells at the later time point. In the following definition, we introduced a relative growth rate function to describe the relationship between the expression profile of a cell and the average number of living descendants it gave rise to after certain amount of time.

Definition 4. A relative growth rate function associated with a temporal coupling is a function g(x)

satisfying

$$\int {γ}_{s , t} ( x , y ) dy = {ℙ}_{s} ( x ) \frac{{g ( x )}^{t - s}}{\int {g ( x )}^{t - s} d {ℙ}_{s} ( x )} .$$

The integral on the left-hand side represented the amount of mass coming out of x and going to any y. The term P(x) on the right hand side accounted for the abundance of cells with expression profile x, and the function g(x) represented the exponential increase in mass per unit time.

Having defined the notion of developmental processes and temporal couplings, we now turned to estimating these from data.

2. The Optimal Transport Principle for Developmental Processes

Single-cell RNA-Seq allowed us to sample cells from a developmental process at various time points, but it did not give any information about the coupling between successive time points. Without making any assumptions, it was impossible to recover the temporal coupling even given infinite data in the form of the full distributions Pₛ and Pₜ. However, we claimed that it was reasonable to assume that cells don't change expression by large amounts over short time scales. This assumption allowed us to estimate the coupling and infer which cells go where.

We began with a simple one-dimensional example to build intuition.

Example 1. Let X₀˜N (0, σ²) and X₁˜N (μ, σ²) be one dimensional Gaussian variables representing the location of a particle at time 0 and at time 1. One simple heuristic to estimate \hat{γ} is to minimize the squared distance that the particle moves from time 0 to time 1:

$$\hat{γ} ← arg {min}_{π} {𝔼}_{π} {‖ {X}_{0} - {X}_{1} ‖}^{2} .$$

We minimized over all couplings π with marginals (0, σ²) and (μ, σ²). One can check that the optimal joint distribution is a two dimensional Gaussian with the following dependence structure:

X₁=X₀+μ.

This heuristic to couple marginals was called optimal transport (OT). If c(x, y) denoted the cost of transporting a unit mass from x to y, and the amount we transferred from x to y is π(x, y), then the total cost of transporting mass according to such a transport plan π is given by

∫∫c(x,y)π(x,y)dxdy.

In this study we focused on the cost defined by the squared-Euclidean distance

c(x,y)=∥x−y∥²,

on an appropriate input space. We made this choice to focus on Wasserstein-2 transport because of the many attractive theoretical properties it enjoyed over Wasserstein-1 transport (Villani, 2008).

The optimal transport plan minimized the expected cost subject to marginal constraints:

$$π ( ℙ , ℚ ) = {minimize}_{π} \int \int c ( x , y ) π ( x , y ) dxdy \\ subject to \int π ( x , \cdot ) dx = ℚ \\ \int π ( \cdot , y ) dy = ℙ . \qquad ( 1 )$$

Note that this was a linear program in the variable π because the objective and constraints were both linear in π. The optimal objective value defined the transport distance between P and Q (it was also called the Earthmover's distance or Wasserstein distance). Unlike many other ways to compare distributions (such as KL-divergence or total variation), optimal transport took the geometry of the underlying space into account. For example, the KL-Divergence was infinite for any two distributions with disjoint support, but the transport distance depended on the separation of the support. For a comprehensive treatment of the rich mathematical theory of optimal transport, we refer the reader to (Villani, 2008).

2.1 the Optimal Transport Principle for Developmental Processes.

We proposed to use optimal transport to estimate the temporal coupling of a developmental process. We made two modifications to classical optimal transport to adapt it to our biological setting.

1. Classical optimal transport had conservation of mass built into the constraints (1). We accounted for growth by rescaling the distribution Pₜ before applying OT.

2. The coupling identified by classical optimal transport was purely deterministic in the sense that each point was transported to a single point. However, for cells whose fates were not completely determined, the true coupling should have a degree of entropy to it. We therefore added a term to the objective to promote entropy in the transport coupling.

Injecting a small amount of entropy also made sense even for a population of cells with truly deterministic descendant distribution. When we sampled finitely many cells at time t₂, the true descendants of any given t₁ cell were not captured. Therefore entropy in the transport map could be used to represent our statistical uncertainty in the inferred descendant distribution.

In order to state the optimal transport principle, we first introduced some notation. Let Pₜ denote a developmental process with temporal coupling γ_{s,t} and with relative growth function g(x). Let Qs denote the distribution obtained by rescaling Pₛ by the relative growth rate:

$${ℚ}_{s} ( x ) = {ℙ}_{s} ( x ) \frac{{g}^{t - s} ( x )}{\int {g}^{t - s} ( z ) d {ℙ}_{s} ( z )} .$$

Finally, let π_{s,t}(ϵ) denote the entropy-regularized optimal transport coupling of Qₛ and Pₜ, defined as the solution to the following optimization problem

$${π}_{s , t} ( ϵ ) = {minimize}_{π} \int \int c ( x , y ) π ( x , y ) dxdy - ϵ \int π ( x , y ) log π ( ) dxdy \\ subject to \int π ( x , \cdot ) dx = {ℚ}_{s} \\ \int π ( \cdot , y ) dy = {ℙ}_{t} . \qquad ( 2 )$$

We now stated the optimal transport principle for developmental process

s≈t⇒π_{s,t}(ϵ)≈γ_{s,t}.

In words, over short time scales, the true coupling was well approximated by the OT coupling. In section 3, we show how to estimate π_{s,t}(ϵ) from data (we occasionally omit the dependence on ϵ and write π_{s,t}). This in turn gives us an estimate of γ_{s,t}.

3. Inferring Temporal Couplings from Empirical Data

In this section we showed how to estimate the temporal couplings of a developmental process from data.

Definition 5 (developmental time series). A developmental time series was a sequence of samples from a developmental process Pₜ on R^{G}. This was a sequence of sets S₁, . . . , S_{T}⊂R^{G} collected at times t₁, . . . , t_{T}∈R. Each Sᵢ is a set of expression profiles in R^{G} drawn independently from Pₜ.

From this input data, we formed an empirical version of the developmental process. Specifically, at each time point tᵢ we formed the empirical probability distribution supported on the data x Sᵢ. We summarize this in the following definition:

Definition 6 (Empirical developmental process). An empirical developmental process \hat{P}ₜ is a time vary-ing distribution constructed from a developmental time course S₁, . . . , S_{T}:

$${\hat{ℙ}}_{\text{?}} = \frac{1}{\vert {S}_{i} \vert} {\sum }_{x ∈ {S}_{i}} {δ}_{x} . \\ \text{?} \text{indicates text missing or illegible when filed} \qquad ( 3 )$$

The empirical developmental process was undefined for t∉{t₁, . . . , t_{T}}.

In order to estimate the coupling from time t₁ to time t₂, we first constructed an initial estimate the growth rate function g(x). In practice, we form an initial estimate ĝ(x) as the expectation of a birth-death process on gene expression space with birth-rate β(x) and death rate δ(x) defined in terms of expression levels of genes involved in cell proliferation and apoptosis. We ultimately leveraged techniques from unbalanced transport (Chizat et al., 2017) to refine this initial estimate to learn cellular growth and death rates automatically from data.

We then form the rescaled empirical distribution

$${\hat{ℚ}}_{{t}_{1}} ( x ) = {\hat{ℙ}}_{{t}_{1}} ( x ) \frac{{\hat{g} ( x )}^{{t}_{1} - {t}_{2}}}{\int {\hat{g} ( z )}^{{t}_{1} - {t}_{2}} d {\hat{ℙ}}_{{t}_{\text{?}}} ( z )} , \\ \text{?} \text{indicates text missing or illegible when filed}$$

and compute the optimal transport map \hat{π}ₜ_{₁}_{,t}_{₂} between \hat{Q}ₜ_{₁} and \hat{P}ₜ_{₂}

3.1 Estimating Couplings Between Adjacent Time Points

In order to identify an optimal transport plan connecting \hat{Q}t1 and \hat{P}t2, we solved an optimization problem with a matrix-valued optimization variable. In the classical zero-entropy setting (2) with ϵ=0 was a linear program. While the classical optimal transport linear program could be difficult to solve for large numbers of points, fast algorithms have been recently developed (Cuturi, 2013) to solve the entropically regularized version of the transport program. Entropic regularization speeded up the computations because it made the optimization problem strongly convex, and gradient ascent on the dual could be realized by successive diagonal matrix scalings called Sinkhorn iterations (Cuturi, 2013). These were very fast operations.

The scaling algorithm for entropically regularized transport had also been extended to work in the setting of unbalanced transport (Chizat et al., 2017), where the equality constraints were relaxed to bounds on the marginals of the transport plan (in terms of KL-divergence or total variation or a general f-divergence). In our application this was very attractive from a modeling perspective for the following reasons:

1. We may have specified the growth rate function ĝ(x). Unbalanced transport adjusted the input growth rate in order to reduce the transport cost. This allowed us to automatically learn growth rates from scratch.

2. Even if the growth rates were completely uniform, the random sampling could introduce what looked like growth. For example, suppose there was a rare subpopulation of cells consisting of 5% of the total. If at one time point, we randomly sampled fewer of these cells so that they comprised 4% of the total, and at the next time point we sample 6%, then it would look like this population had increased by 50%. Unbalanced transport could automatically adjust for this apparent growth.

We used both entropic regularization and unbalanced transport. To compute the transport map between the empirical distributions of expression profiles observed at time tᵢ and tᵢ₊₁, we solved the following optimization problem

$${\hat{π}}_{i \text{?} , {t}_{i + 1}} = {arg min}_{π} {\sum }_{x ∈ {S}_{i}} {\sum }_{y ∈ {S}_{i + 1}} c ( x , y ) π ( x , y ) - ϵ \int π ( x , y ) log π ( x , y ) dxdy \\ subject to KL [ {\sum }_{x ∈ {S}_{i}} π ( x , y ) \vert \vert d {\hat{ℙ}}_{{t}_{i + 1}} ( y ) ] \leq \frac{1}{{λ}_{1}} \\ KL [ {\sum }_{y ∈ {S}_{i + 1}} π ( x , y ) \vert \vert d {\hat{ℚ}}_{{t}_{i}} ( x ) ] \leq \frac{1}{{λ}_{2}} \\ \text{?} \text{indicates text missing or illegible when filed} \qquad ( 4 )$$

where ϵ, λ₁ and λ₂ are regularization parameters.

This is a convex optimization problem in the matrix variable π∈ [symbol] ^{N}^{ᵢ}^{×N}^{ᵢ₊₁}. here. Nᵢ=|Sᵢ| is is the number of cells sequenced at time ti. It takes about 5 seconds to solve this unbalanced transport problem using the scaling algorithm of (Chizat et al., 2017) on a standard laptop with Ni≈5000.

Note that by default the densities (on the discrete set Si) of the empirical distributions specified in equation (3) are simply

$$d {\hat{ℙ}}_{{t}_{i}} ( x ) = \frac{1}{{N}_{i}} .$$

However, in principle one could use nonuniform empirical distributions (e.g., if one wanted to include information about cell quality).

To summarize: given a sequence of expression profiles S₁, . . . , S_{T}, we solved the optimization problem (4) for each successive pair of time points Sᵢ, Sᵢ₊₁. For the pair of timepoints (tᵢ, tᵢ₊₁), this gave us a transport map \hat{π}ₜ_{ᵢ}_{,t}_{ᵢ₊₁}. With enough data, this may be a good estimate of πₜ_{ᵢ}_{,t}_{ᵢ₊₁} because it is well known that transport maps are consistent in the sense that

$${lim}_{N \text{?} N \text{?} \to ∞} {\hat{π}}_{{t}_{i} , {t}_{i + 1}} = {π}_{{t}_{\text{?}} , {i}_{i + 1}} . \\ \text{?} \text{indicates text missing or illegible when filed}$$

Taken together with the optimal transport principle: πₜ_{ᵢ}_{,t}_{ᵢ₊₁}≈γₜ_{ᵢ}_{,t}_{ᵢ₊₁},

We therefore could estimate γₜ_{ᵢ}_{,t}_{ᵢ₊₁} from \hat{π}ₜ_{ᵢ}_{,t}_{ᵢ₊₁} when Ni is large enough.

3.2 Estimating Long-Range Couplings

We relied on an assumption of Markovity (or memorylessness) in order to estimate couplings over longer time intervals. Recall that a stochastic process was Markov if the future was independent of the past, given the present. Equivalently, it was fully specified by the couplings between pairs of time points. We defined Markov developmental processes in a similar spirit:

Definition 7 (Markov developmental process). A Markov developmental process Pₜ is a time-varying distribution on R^{G} that is completely specified by couplings between pairs of time points in the following sense. For any three time points s<t<τ, the long-range coupling γ_{s,τ} was equal to the composition of short-range couplings: γ_{t,τ}oγ_{s,t}=γ_{s,τ}.

Note that the optimal transport maps \hat{π}s,t did not have this compositional property. Composing the OT coupling from time s to t and then from t to τ was not the same as optimally transporting from s directly to τ. In general, we do not recommend computing OT maps directly between non-adjacent time points. We leveraged the Markovity assumption to estimate couplings over long time intervals by composing estimates over shorter intervals. Formally, for any pair of time points tᵢ, tᵢ₊ₖ, we estimate the coupling \hat{γ}ₜ_{ᵢ}_{,t}_{ᵢ₊ₖ} by composing as follows:

These compositions were computed via ordinary matrix multiplication.

It is an interesting question to what extent developmental processes are Markov. On gene expression space, they were likely not strictly Markov because, for example, the history of gene expression could influence chromatin modifications, which may not themselves be fully reflected in the observed expression profile but could still influence the subsequent evolution of the process. However, it was possible that developmental processes could be considered Markov on some augmented space. Note that our core technique for estimating a single temporal coupling over a short time interval does not rely on any Markov assumption.

4. Interpreting Transport Maps

In the previous section we introduced the principle of optimal transport for time series of gene expression profiles. Given a time series of expression profiles S₁, . . . , S_{T}, we used this principle to compute a sequence of transport maps between subsequent time slices. In this section we define the ancestors and descendants of any subset of cells from this sequence of transport maps in section 4.1. Then, in section 4.2 we explain an intuitive physical interpretation of entropy-regularization. Finally, in section 4.3 we describe a connection between optimal transport, gradient flows, and Waddington's landscape.

4.1 Defining Ancestors, Descendants and Trajectories

We defined the descendants and ancestors of subgroups of cells evolving according to a Markov (i.e. memoryless) developmental process.

Our definition of ancestors and descendants relies on a notion of pushing sets of cells through a trans-port map. Before defining ancestors and descendants, we introduce this terminology. As a distribution on the product space R^{G}×R^{G}, a coupling γ assigns a number γ(A, B) to any pair of sets A, B⊂R^{G}

γ(A,B)=∫_{x∈A}∫_{y∈B}γ(x,y)dxdy.

This number π(A, B) represented the amount of mass coming from A and going to B. When we did not specify a particular destination, the quantity γ(A,) specified the full distribution of mass coming from A. We referred to this action as pushing A through the transport plan γ. More generally, we could also push a distribution p forward through the transport plan γ via integration

[symbol]

μ [symbol] ∫γ(x,⋅)dμ(x).

We refer to the reverse operation as pulling a set B back through γ. The resulting distribution γ(⋅,B) encodes the mass ending up at B. We can also pull distributions μ back through γ in a similar way:

μ [symbol] ∫γ(⋅,y)dμ(y).

We sometimes refer to this as back-propagating the distribution μ (and to pushing μ forward as forward propagation).

Equipped with this terminology, we define ancestors and descendants as follows:

Definition 8 (descendants in a Markov developmental process). Consider a set of cells C⊂ [symbol] ^{G} which lived at time t₁ were part of a population of cells evolving according to a [symbol] Markov developmental process Pₜ. Let γt₁,t₂ denote the coupling from time t₁ to time t₂. The descendants of C at time t₂ are obtained by pushing C through γ.

Definition 9 (ancestors in a Markov developmental process). Consider a set of cells C⊂ [symbol] ^{G}, which lived at time t₂ and were part of a population of cells evolving according to a Markov developmental process Pₜ. Let π denote the transport map for Pₜ from time t₂ to time t₁. The ancestors of C at time t₁ were obtained by pulling C back through y.

Trajectories: We defined to the ancestor trajectory to a set C as the sequence of ancestor distributions at earlier time points. Similarly, we refer to the descendant trajectory from a set C as the sequence of descendant distributions at later time points.

4.2 A Physical Interpretation of Entropy Regularized Optimal Transport

In this section we explain an interesting physical interpretation of entropy-regularized optimal transport. Consider a collection of N indistinguishable particles undergoing Brownian motion with diffusion coefficient ϵ. Suppose we observe the N particle positions at time 0 and at time 1. If N=1, the distribution on paths connecting the starting and ending point is called a Brownian bridge. For N>1, the distribution over paths involves two components:

1. A coupling of the particles specifying which particle goes where (because the particles are indistinguishable, this is not uniquely specified by the observations).

2. Given a matching, the distribution on paths for each matched pair is a Brownian bridge.

The coupling was a random permutation that matched points at time 0 to points at time 1. The distribution of this random permutation depends on the variance of the Brownian motion. It turned out that the expected (i.e. average) coupling could be computed by maximum entropy optimal transport. These ideas could be traced back to Schrodinger's 1932 work in statistical electrodynamics (Schrodinger, 1932), but the connection to optimal transport was not made explicit until recently (Le'onard, 2014). We summarize this in the following theorem:

Theorem 1. Entropy regularized optimal transport gives the expectation of the distribution over cou-plings induced by Brownian motion (when the diffusion coefficient of the Brownian motion is equal to the entropy regularization parameter).

4.3 Gradient Flow and Waddington's Landscape

In this section we show how optimal transport can be interpreted as a gradient flow in gene expression space (capturing cell-autonomous processes) or in the space of distributions (capturing cell-nonautonomous processes). For a full treatment of the rich OT theory of gradient flows, we refer the reader to (Ambrosio et al., 2005; Santambrogio, 2015).

We began by considering the simple setting described by Waddington's landscape, which described a gradient flow in gene expression space and is a special case of what we could capture with optimal transport. Mathematically, Waddington's landscape defined a potential function Φ assigning potential energy Φ(x) to a cell with expression profile x. The cells roll eddownhill according to the gradient of Φ to describe a trajectory x(t) satisfying the differential equation

$$\frac{dx}{dt} = - ∇ Φ ( x ) . \qquad ( 5 )$$

This equation governing the trajectory of individual cells induced a flow in the distribution of the population of cells:

$$\frac{d {ℙ}_{t}}{dt} = div [ ∇ Φ ( x ) {ℙ}_{i} ] . \qquad ( 6 )$$

Intuitively, this equation stated that the change in mass for each small volume of space (on the left-hand side) was equal to the flux of mass in and out (given by the divergence on the right hand side).

Optimal transport can capture this type of potential driven dynamics: the true coupling specified by (5) is close to the optimal transport coupling over short time scales. To motivate this, we appeal to a classical theorem establishing a dynamical formulation of optimal transport.

Theorem 2 (Benamou and Brenier, 2001). The optimal objective value of the transport problem (1) is equal to the optimal objective value of the following optimization problem

$${minimize}_{ρ , v} {\int }_{0}^{1} {\int }_{{ℝ}^{G}} {‖ v ( t , x ) ‖}^{2} ρ ( t , x ) dtdx \\ subject to ρ ( 0 , \cdot ) = ℙ , ρ ( 1 , \cdot ) = ℚ . \\ ∇ \cdot ( ρ v ) = \frac{\partial ρ}{\partial t} \qquad ( 7 )$$

In this theorem, v was a vector-valued velocity field that advected the distribution ρ from P to Q, and the objective value to be minimized was the kinetic energy of the flow (mass×squared velocity). In our setting, the two distributions were snapshots Pₛ and Pₜ of a developmental process at two time points, and the theorem showed that the transport map π_{s,t} could be seen as a point-to-point summary of a least-action continuous time flow, according to an unknown velocity field. In the special case when the velocity field was the gradient of a potential Φ (i.e. Waddington landscape), the theorem implied that the coupling (5) achieved the optimal transport cost. In other words, OT could capture potential driven dynamics. In addition, optimal transport could also describe much more general settings. This velocity field could change over time and also depended on the entire distribution of cells, so optimal transport could describe very general developmental processes including those with cell-cell interactions, as described below.

We showed that the evolution (6) was a special case of a Wasserstein gradient flow to minimize the linear energy functional

E( [symbol] )=∫Φ(x)d [symbol] (x).

We then described non-linear gradient flows, which can capture cell-cell interactions. To understand gradient flows, we started with the familiar notion of gradient descent:

xₖ₊₁=−η∇E(xₖ)+xₖ.

This was rewritten as a proximal procedure, where one seeks to minimize E over all x in the proximity of xₖ

$${x}_{k + 1} = {argmin}_{x} E ( x ) + \frac{1}{2 η} {‖ x - {x}_{k} ‖}^{2} . \qquad ( 8 )$$

We performed a similar proximal procedure in the space of distributions, replacing the Euclidean norm ∥⋅∥² with the Wasseerstein distance:

$${ℙ}_{k + 1} = {argmin}_{ρ} E ( ρ ) + \frac{1}{2 η} {W}_{2}^{2} ( ρ , {ℙ}_{k} ) . \qquad ( 9 )$$

This produced a sequence of iterates P₀, P₁, . . . , Pₖ. The gradient flow was the limit obtained as we shrink the step-size n↓0. In (Richard Jordan and Otto, 1998), it's proven that for the linear energy functional

E( [symbol] )=∫Φ(x)d [symbol] (x),

the limiting gradient flow converges to a solution of (6).

Going beyond the linear energy functional associated with Waddington's landscape, one could describe cell-cell interactions with an interaction energy of the form

E( [symbol] )=∫∫I(x,y)d [symbol] (x)d [symbol] (y).

Gradient flows for interaction potentials are discussed in chapter 7 of (Santambrogio, 2015).

Learning models of gene regulation Motivated by this interpretation of optimal transport as a gradient flow according to an unknown vector field, we described a strategy to estimate such a vector field from data in Waddington-OT: Concepts and Implementation. We interpreted the vector field as a model of gene regulation—it predicted gene expression at later time points as a function of transcription factor expression at current time points. We assumed that the vector field did not change over time, and described a cell-autonomous flow, but we do not assume that it comes from a potential function.

II. WADDINGTON-OT: Concepts and Implementation

Building on the theoretical foundations developed in Modeling developmental processes with optimal transport, we developed WADDINGTON-OT: our method for computing ancestor and descendant trajectories, interpolating developmental processes, inferring gene regulatory models, and visualizing developmental landscapes. We begin with an overview in Section 1, and we then describe the specific details in Sections 2-8.

1. Overview

To apply WADDINGTON-OT to a new dataset. The code is available on GitHub: https://github.com/broadinstitute/wot/

In the sections below we describe our procedures for computing transport maps, computing trajectories to cell sets, fitting local and global regulatory models, visualizing the developmental landscape, interpolating the distribution of cells at held-out time points.

To keep the focus here general-purpose, we deferred all reprogramming-specific details to the subsequent sections Methods.

Input data: The input to our suite of methods was a temporal sequence of single cell gene expression matrices, prepared as described in Preparation of expression matrices.

Computing transport maps: Waddington-OT calculated transport maps between consecutive time points and automatically estimated cellular growth and death rates. In Section 2 below we provide guidelines for defining the cost function, selecting regularization parameters and (optionally) providing an initial estimate of growth and death rates.

Ancestors, descendants, and trajectories: We describe in Section 3 how we computed trajectories plot trends in gene expression. Briefly, the developmental trajectory of a subpopulation of cells refers to the sequence of ancestors coming before it and descendants coming after it. Using the transport maps, we calculated the forward or backward transport probabilities between any two classes of cells at any time points. For example, we took successfully reprogrammed cells at day 18 and use back-propagation to infer the distribution over their precursors at day 17.5. We then propagated this back to day 17, and so on to obtain the ancestor distributions at all previous time points. This was the developmental trajectory to iPS cells. We plotted trends in gene expression over time.

Fitting regulatory models: We describe our method to fit a regulatory model to the transport maps in Section 4. Transcription factors (TFs) that appeared to play important roles along trajectories to key destinations were identified by two approaches. The first approach involved constructing a global regulatory model. Pairs of cells at consecutive time points were sampled according to their transport probabilities; expression levels of TFs in the cell at time t were used to predict expression levels of all non-TFs in the paired cell at time t+1, under the assumption that the regulatory rules are constant across cells and time points. (TFs were excluded from the predicted set to avoid cases of spurious self-regulation). The second approach involved local enrichment analysis. TFs were identified based on enrichment in cells at an earlier time point with a high probability (>80%) of transitioning to a given fate vs. those with a low probability (<20%).

Visualizing the developmental landscape To visualize the developmental landscape, we first reduced the dimensionality of the data with diffusion components, and then embedded the data in two dimensions with force-directed graph visualization (as described in Section 5). While alternative visualization methods, such as t-distributed Stochastic Neighbor Embedding (t-SNE), were well suited for identifying clusters, they did not preserve global structures relevant to studying trajectories across a time course. FLE better reflected global structures by including repulsive forces between dissimilar points. In particular, these repulsive forces seemed to do a good job of splaying out the spikes present in the diffusion map embedding.

Geodesic interpolation: To validate the temporal couplings, Waddington-OT could interpolate the distribution of cells at a held-out time point. The method wsa performing well if the interpolated distribution was close to the true held-out distribution (compared to the distance between different batches of the held-out distribution). Otherwise, it was possible that the method requires more data or finer temporal resolution.

Section 6 describes our method to interpolate the distribution of cells at a held-out time point. Our validation results for IPS reprogramming are presented in the subsequent section on Validation by geodesic interpolation. We performed extensive sensitivity analysis to show that our temporal couplings produce valid interpolations over a wide range of parameter settings perturbations to the data (down sampling cells or reads). See QUANTIFICATION AND STATISTICAL ANALYSIS for this sensitivity analysis.

2. Computing transport maps

Recall that for any pair of time points we computed a transport plan that minimizes the expected cost of re-distributing mass, subject to constraints involving the relative growth rate (see Modeling developmental processes with optimal transport for a precise statement of the optimization problem). To compute these transport matrices, we needed to specify a cost function, numerical values for the regularization parameters, and (optionally) an initial estimate for the relative growth rate.

2.1 Cost function

To compute the cost of transporting each individual point x from time t₁ to position y at time t₂, we first performed principal components analysis (PCA) on the data from this pair of time points to reduce to 30 dimensions. This dimensionality reduction was performed separately for each pair of adjacent time points. We defined the cost function to be squared Euclidean distance in this ‘local-PCA space’.

Finally, we normalized the cost matrix by dividing each entry by the median cost for that time interval. Here the cost matrix was the matrix with entries C_{i,j}=c(xᵢ, yⱼ) for each xi form time t₁ and yⱼ at time t₂. This rescaling of the cost allowed us to refer to specific numerical values of the regularization parameters, without worrying about the global scale of distances.

2.2 Regularization Parameters

The optimization problem (4) involved three regularization parameters:

1. The entropy parameter E controlled the entropy of the transport map. An extremely large entropy parameter gave a maximally entropic transport map, and an extremely small entropy parameter gave a nearly deterministic transport map. The default value was 0.05.

2. λ₁ controlled the degree to which transport was unbalanced along the rows. Large values of λ₁ imposed stringent constraints related to relative growth rates. Small values of λ₁ gave the algorithm more flexibility to change the relative growth rates in order to improve the transport objective. The default value was 1. To visually inspect the degree of unbalancedness, we recommend plotting the input row-sums vs the output row-sums of the transport map (See FIGS. 30A-30G).

3. λ₂ controlled the degree to which transport is unbalanced along the columns. The default value was λ₂=50. This large value essentially imposed equality constraints for the column marginals. A smaller value of λ₂ would allow different amounts of mass to transport to some cells at time t₂. We recommend keeping a large value for λ₂ so that the results are balanced along the columns. To visually inspect the degree of unbalancedness, one can plot the input column-sums vs the output column-sums of the transport map.

As we demonstrate in QUANTIFICATION AND STATISTICAL ANALYSIS, our validation results were stable over a wide range of values for E and λ₁.

2.3 Estimating Relative Growth Rates

Our method solved the optimization problem (4) several times, using the output row-sums of the optimal transport map \hat{π}t1,t2 as a new estimate for the relative growth rate function ĝ(x). By default, we initialize with ĝ(x)=1, so that all cells growed at the same rate. With some prior knowledge of growth rates (e.g. based on gene signatures of proliferation and apoptosis), this could be incorporated in the initial estimate for ĝ(x). For our reprogramming data, we showed how we formed an initial estimate for relative growth rates in Estimating growth and death rates and computing transport maps.

3 Ancestors, Descendants, and Trajectories

Recall that the transport map \hat{π}_{t1, t2} connecting cells from time t₁ to cells from time t₂ has a row for each cell x at time t₁ and a column for each cell y at time t₂. Each row specifies the descendant distribution of a single cell x from time t₁. The descendant mass is the sum of all the entries across a row. This row-sum was proportional to the number of descendants that x would contribute to the next time point. Intuitively, the descendant distribution specified which cells at time t₂ were likely to be descendants of x (see section 4.1 of Modeling developmental processes with optimal transport for the formal definition of descendants in a developmental process).

Similarly, each column specified the ancestor distribution of a cell y from time t₂. The ancestor mass was usually the same for each cell y. The ancestor distribution told us which cells at time t₁ were likely to give rise to the cell y.

Given a set of cells C, we computed the descendant distribution of the entire set by adding the descendant distributions of each cell in the set. This was computed efficiently via matrix multiplication as follows: Let S₁ donote all the cells from time point t1, and let

$$p ( x ) = \{ \begin{matrix}1 & x ∈ C \\ 0 & otherwise\end{matrix}$$

denote the uniform distribution on C⊂S. The descendant distribution of C was given by \hat{π}t1,t2 p. One could compute ancestor distributions in a similar way

After computing the trajectory to or from a cell set C (in the form of a sequence of ancestor and descendant distributions), we computed trends in expression for any gene or gene signature along the trajectory. For each time point, we simply computed the mean expression weighting each cell according to the probability distribution defined by the ancestor or descendant distribution.

4. Learning Gene Regulatory Models

In this section we describe two strategies to summarize the transport maps by learning models of gene regulation. The first model we describe is a simple local enrichment analysis to identify transcription factors (TFs) enriched in ancestors of a set of cells. The second model is motivated by the dynamical systems formulation of optimal transport, as described above in Section 4.3.

4.1 Local Model: TF Enrichment Analysis of Top Ancestors

We performed local enrichment analysis as follows. Given a set of cells C at time t₂, we first computed the ancestor distribution of C at an earlier time t₁, as described in Section 3 above. We then selected cells contributing the most mass to the ancestor distribution, until a certain amount of mass was accounted for (e.g. 30% of the ancestor mass). We referred to these as the top ancestors at time t₁ of the cell set C. Finally, we compared the top ancestors to a null set of cells from the same time point. For example, this null cell set could be:

all cells except for the top ancestors,

the bottom ancestors (defined to be all cells except for the top ancestors of a less-strict cut-off),

the bottom ancestors restricted to a specialized subset (e.g. all other trophoblasts when C is a specific subset of trophoblasts like spongiotrophoblasts).

4.2 Global Model: Learning a Cell-Autonomous Gradient Flow

To learn a simple description of the temporal flow, we assumed that a cell's trajectory was cell-autonomous and, in fact, depended only on its own internal gene expression. We knew this was wrong as it ignored paracrine signaling between cells, and we returned to discuss models that include cell-cell communication at the end of this section. However, this assumption is powerful because it exposes the time-dependence of the stochastic process Pₜ as arising from pushing an initial measure through a differential equation:

\dot{x}==ƒ(x). (10)

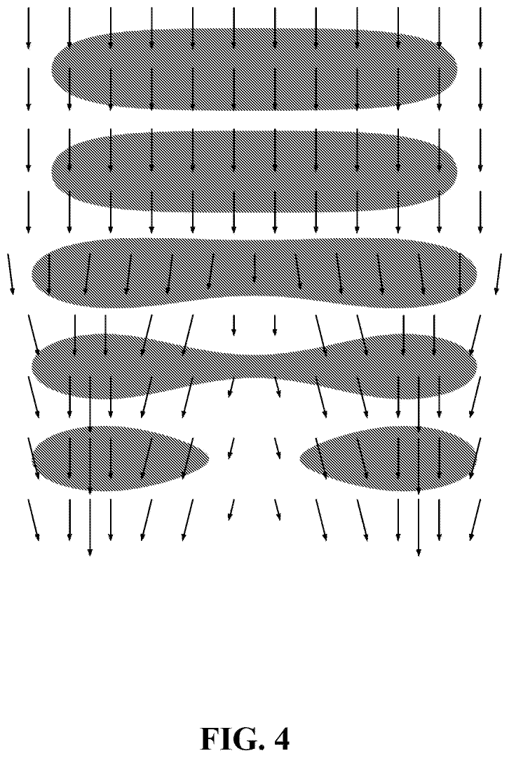

Here ƒ was a vector field that prescribes the flow of a particle x (see FIG. 4 for a cartoon illustration of a distribution flowing according to a vector field). Our biological motivation for estimating such a function ƒ was that it encoded information about the regulatory networks that created the equations of motion in gene-expression space.

We set up a regression to learn a regulatory function ƒ that models the fate of a cell at time tᵢ₊₁ as a function of its expression profile at time tᵢ. Our approach involved sampling pairs of points using the couplings from optimal transport:

For each pair of time points tᵢ, tᵢ₊₁, we sampled pairs of cells (Xₜ_{ᵢ}, Xₜ_{ᵢ₊₁}) from the joint distribution specified by the transport map \hat{π}ₜ_{ᵢ},ₜ_{ᵢ₊₁}.

Using the training data generated in the first step, we set up the following regression:

$${min}_{f ∈ ℱ} {𝔼}_{{\hat{π}}_{{t}_{i} , {t}_{i + 1}}} {‖ {X}_{t + 1} - f ( {X}_{{t}_{i}} ) ‖}^{2} ,$$

where [symbol] was a rectified-linear function class defined in terms of a specific generalized logistic function l: [symbol] :

$$𝓁 ( x ; k , b , {y}_{0} , {x}_{0} ) = \frac{{ky}_{0}}{{y}_{0} + ( k - {y}_{0} ) {e}^{- b ( x - {x}_{0} )}} ,$$

where k, b, y0, z0∈ [symbol] were parameters of the generalized logistic function l(x).

We define a function class [symbol] consisting of functions ƒ: [symbol] ^{G}→ [symbol] ^{G} of the form

ƒ(x)=U [symbol] (WTx),

where l was applied entry-wise to the vector WTx∈ [symbol] ^{M} to obtain a vector that we multiplied against U∈ [symbol] ^{G×M}. Here T∈ [symbol] ^{G}^{_{TF}}^{×G} denoted a projection operator that selected only the coordinated of x that were transcription factors, and G_{TF} was the number of transcription factors. This gave a set of low-rank, linear functions with sparse factors. Each rank-1 component was interpreted as a regulatory module of transcription factors acting on a module of regulated genes.

We set up the following optimization over matrices

$${min}_{U , W} {𝔼}_{r} {‖ \frac{{X}_{{t}_{i}} - {X}_{{t}_{i + 1}}}{{Δ}_{t}} - U 𝓁 ( {WTX}_{{t}_{i}} ) ‖}^{2} + {η}_{1} {‖ U ‖}_{1} + {η}_{2} {‖ W ‖}_{1} , + {η}_{3} {‖ W ‖}_{2}^{2} \\ s . t . U \geq 0. \qquad ( 11 )$$

where (Xₜᵢ, Xₜᵢ₊₁) is a pair of random variables distributed according to the normalized transport map r, and ∥U∥₁ denotes the sparsity-promoting ƒ₁ norm of U, viewed as a vector (that is, the sum of the absolute value of the entries of U). Each rank one component (row of U or column of W) gives us a group of genes controlled by a set of transcription factors. The regularization parameters η₁ and η₂ control the sparsity level (i.e. number of genes in these groups).

Implementation: We designed a stochastic gradient descent algorithm to solve (11). Over a sequence of epochs, the algorithm sampled batches of points (Xₜᵢ, Xₜᵢ₊₁) from the transport maps, computed the gradient of the loss, and updates the optimization variables U and W. The batch sizes were determined by the Shannon diversity of the transport [symbol] maps: for each pair of consecutive time points, we computed the Shannon diversity S of the transport map, then randomly sampled max(S 10⁻⁵, 10) pairs of points to add to the batch. We ran for a total of 10,000 epochs.

Cell non-autonomous processes: We concluded our treatment of gene regulatory networks by discussing an approach to cell-cell communication. Note that the gradient flow (10) only made sense for cell autonomous processes. Otherwise, the rate of change in expression x was not just a function of a cell's own expression vector x(t), but also of other expression vectors from other cells. We accommodated cell non-autonomous processes by allowing ƒ to also depend on the full distribution Pₜ:

$$\frac{dx}{dt} = f ( x , {ℙ}_{t} ) . \qquad ( 12 )$$

Concretely, we could allow ƒ to depend on the mean expression levels of specific genes (expressed by any cell) encoding, for example, secreted factors or direct protein measurements of the factors themselves.

5. Geodesic Interpolation

Optimal transport provided an elegant way to interpolate distribution-valued data, analogous to how linear regression can be used to interpolate numerical or vector-valued data. Given two numerical data-points, a simply way to interpolate was to connect them with a line; this was the shortest path connecting the observed data. Given two distributions, we interpolated by finding the shortest path in the space of distributions. To do this we needed a notion of distance between distributions, and for this we use the metric induced by optimal transport. This metric space was called Wasserstein space, and this form of interpolation was called geodesic interpolation (Villani, 2008).

We derived a modified version of geodesic interpolation that took into account cell growth. Ordinarily, an interpolating distribution was computed by first computing a transport map between the distributions, and then connecting each point in the first distribution to points in the second according to the transport map. Finally, an interpolating point cloud was produced by from the midpoints of those line segments. (More generally, instead of taking just midpoints, one could also construct a family of interpolations that sweep from the first distribution to the second). We extended this framework to accommodate growth by changing the mass of the point we placed at the midpoint (to account for the fact that cells would have a different number of descendants at time t₁ than they would at time t₂).

Specifically, to interpolate at time sϵ(t₁, t₂) we first renormalize the rows of the transport map so they sum to roughly

$$\frac{{\hat{g} ( x )}^{s - {t}_{1}}}{\int {\hat{g} ( x )}^{s - {t}_{1 }} d {\dot{ℙ}}_{{t}_{1}}}$$

instead of

$$\frac{{\hat{g} ( x )}^{{t}_{2} - {t}_{1}}}{\int {\hat{g} ( x )}^{{t}_{2} - {t}_{1}} d {\dot{ℙ}}_{{t}_{1}} ( x )} .$$

This took
into account the descendant mass each cell would have by time s instead of by time t₂. We then sampled points z₁, . . . , z_{N} as follows:

1. Sampling a pair of points (x, y) from the joint distribution specified by the transport map.

2. Identifying the point

z=αx+(1−α)y

along the line segment connecting x and y. Here a is given by s=αt₁+(1−α)t₂.

By repeating the steps above, we accumulate a point-cloud of points z₁, . . . , z_{N}. Finally, we define the interpolating distribution as

$$\hat{ℙ} ( s ) = \frac{1}{N} {\sum }_{i = 1}^{N} {δ}_{{z}_{i}} .$$

Equipped with this notion of interpolation, we tested the performance of optimal transport by comparing the interpolated distribution to held-out time points. Using the data from time ti and ti+2, we interpolated to estimate the distribution Pti+1. We then computed the Wasserstein distance between the interpolated distribution and the observed distribution. We compared this distance to a null model generated from the independent coupling where we sample pairs (x, y) independently x˜ [symbol] ₜ_{ᵢ} and y˜ [symbol] ₜ_{ᵢ₊₂} in step 1 above. We also compared the interpolated distance to distance between batches of [symbol] tᵢ₊₁. Optimal transport was performing well if the interpolated point cloud was as close to the batches of the held out time point as the batches were to each other, and the null-interpolated point cloud was farther away.

### BIBLIOGRAPHY

Ambrosio, L., Gigli, N., and Savare, G. (2005). Gradient Flows: In Metric Spaces and in the Space of Probability Measures. Lectures in Mathematics. ETH Zürich. Birkhäuser Basel.
Bastian, M., Heymann, S., Jacomy, M., et al. (2009). Gephi: an open source software for exploring and manipulating networks. Icwsm, 8:361-362.
Beygelzimer, A., Kakadet, S., Langford, J., Arya, S., Mount, D., Li, S., and Li, M. S. (2015). Package FNN.
Chizat, L., Peyré, G., Schmitzer, B., and Vialard, F.-X. (2017). Scaling algorithms for unbalanced transport problems. Mathematics of Computation.
Cuturi, M. (2013). Sinkhorn distances: Lightspeed computation of optimal transportation distances. In
Neural Information Processing Systems (NIPS).
Jacomy, M., Venturini, T., Heymann, S., and Bastian, M. (2014). Forceatlas2, a continuous graph layout algorithm for handy network visualization designed for the gephi software. PloS one, 9:e98679.
Léonard, C. (2014). A survey of the schrödinger problem and some of its connections with optimal transport. Discrete and Continuous Dynamical Systems—Series A (DCDS-A), 34(4):1533-1574.
Porpiglia, E., Samusik, N., Van Ho, A. T., Cosgrove, B. D., Mai, T., Davis, K. L., Jager, A., Nolan, G. P., Bendall, S. C., Fantl, W. J., et al. (2017). High-resolution myogenic lineage mapping by single-cell mass cytometry. Nature Cell Biol., 19:558-567.
Richard Jordan, D. K. and Otto, F. (1998). The variational formulation of the fokker. SIAM J. Math. Anal., 29(1):1-17.
Samusik, N., Good, Z., Spitzer, M. H., Davis, K. L., and Nolan, G. P. (2016). Automated mapping of phenotype space with single-cell data. Nature methods, 13:493-496.
Santambrogio, F. (2015). Optimal Transport for Applied Mathematicians: Calculus of Variations, PDEs, and Modeling. Progress in Nonlinear Differential Equations and Their Applications. Springer Inter-national Publishing.
Schrodinger, E. (1932). Sur la theorie relativiste de l'electron et l'interpretation de la mecanique quan-tique. Ann. Inst. H. Poincare, 2:269-310.
Villani, C. (2008). Optimal Transport Old and New. Springer.
Zunder, E. R., Lujan, E., Goltsev, Y., Wernig, M., and Nolan, G. P. (2015). A continuous molecular roadmap to ipsc reprogramming through progression analysis of single-cell mass cytometry. Cell Stem Cell, 16:323-337.

III Experimental methods

1. Derivation of secondary MEFs

OKSM secondary Mouse embryonic fibroblasts (MEFs) were derived from E13.5 female embryos with a mixed B6; 129 background. The cell line used in this study was homozygous for ROSA26-M2rtTA, homozygous for a polycistronic cassette carrying Oct4, Klf4, Sox2, and Myc at the Colla1 locus and homozygous for an EGFP reporter under the control of the Oct4 promoter (Stadtfeld et al., 2010). Briefly, MEFs were isolated from E13.5 embryos from timed-matings by removing the head, limbs, and internal organs under a dissecting microscope. The remaining tissue was finely minced using scalpels and dissociated by incubation at 37° C. for 10 minutes in trypsin-EDTA (Thermo Fisher Scientific). Dissociated cells were then plated in MEF medium containing DMEM (Thermo Fisher Scientific), supplemented with 10% fetal bovine serum (GE Healthcare Life Sciences), non-essential amino acids (Thermo Fisher Scientific), and GlutaMAX (Thermo Fisher Scientific). MEFs were cultured at 37° C. and 4% CO₂ and passaged until confluent. All procedures, including maintenance of animals, were performed according to a mouse protocol (2006N000104) approved by the MGH Subcommittee on Research Animal Care.

2. Derivation of Primary MEFs

Primary MEFs were derived from E13.5 embryos with a B6.Cg-Gt(ROSA)^{26Sortm1(rtTA*M2)Jae}/JxB6; 129S4-Pou5f1^{tm2Jae}/J background. The cell line was homozygous for ROSA26-M2rtTA, and homozygous for an EGFP reporter under the control of the Oct4 promoter. MEFs were isolated as mentioned above.

3. Reprogramming Assay

For the reprogramming assay, 20,000 low passage MEFs (no greater than 3-4 passages from isolation) were seeded in a 6-well plate. These cells were cultured at 37° C. and 5% CO₂ in reprogramming medium containing KnockOut DMEM (GIBCO), 10% knockout serum replacement (KSR, GIBCO), 10% fetal bovine serum (FBS, GIBCO), 1% GlutaMAX (Invitrogen), 1% nonessential amino acids (NEAA, Invitrogen), 0.055 mM 2-mercaptoethanol (Sigma), 1% penicillin-streptomycin (Invitrogen) and 1,000 U/ml leukemia inhibitory factor (LIF, Millipore). Day 0 medium was supplemented with 2 μg/mL doxycycline Phase-1(Dox) to induce the polycistronic OKSM expression cassette. Medium was refreshed every other day. At day 8, doxycycline was withdrawn, and cells were transferred to either serum-free 2i medium containing 3 μM CHIR99021, 1 μM PD0325901, and LIF (Phase-2(2i)) (Ying et al., 2008) or maintained in reprogramming medium (Phase-2(serum)). Fresh medium was added every other day until the final time point on day 18. Oct4-EGFP positive iPSC colonies should start to appear on day 10, indicative of successful reprogramming of the endogenous Oct4 locus.

4. Sample Collection

We profiled a total of 315,000 cells from two time-course experiments across 18 days in two different culture conditions: in the first we profiled ˜65,000 cells collected over 10 time points separated by ˜48 hours; in the second we profiled ˜250,000 cells collected over 39 time points separated by ˜12 hours across an 18-day time course (and every 6 hours between days 8 and 9). In the larger experiment, duplicate samples were collected at each time point. Cells were also collected from established iPSCs cell lines reprogrammed from the same MEFs, maintained either in Phase-2(2i) conditions or in Phase-2(serum) medium. For all time points, selected wells were trypsinized for 5 mins followed by inactivation of trypsin by addition of MEF medium. Cells were subsequently spun down and washed with 1×PBS supplemented with 0.1% bovine serum albumin. The cells were then passed through a 40 micron filter to remove cell debris and large clumps. Cell count was determined using Neubauer chamber hemocytometer to a final concentration of 1000 cells/μl.

5. Single-Cell RNA-Seq

ScRNA-seq libraries were generated from each time point using the 10× Genomics Chromium Controller Instrument (10× Genomics, Pleasanton, Calif.) and Chromium-Single Cell 3′ Reagent Kits v1 (˜65,000 cells experiment) and v2 (˜250,000 experiment) according to manufacturer's instructions. Reverse transcription and sample indexing were performed using the C1000 Touch Thermal cycler with 96-Deep Well Reaction Module. Briefly, the suspended cells were loaded on a Chromium controller Single-Cell Instrument to first generate single-cell Gel Bead-In-Emulsions (GEMs). After breaking the GEMs, the barcoded cDNA was then purified and amplified. The amplified barcoded cDNA was fragmented, A-tailed and ligated with adaptors. Finally, PCR amplification was performed to enable sample indexing and enrichment of the 3′ RNA-Seq libraries. The final libraries were quantified using Thermo Fisher Qubit dsDNA HS Assay kit (Q32851) and the fragment size distribution of the libraries were determined using the Agilent 2100 BioAnalyzer High Sensitivity DNA kit (5067-4626). Pooled libraries were then sequenced using Illumina Sequencing. All samples were sequenced to an average depth of 87 million paired-end reads per sample (see Experimental Methods), with 98 bp on the first read and 10 bp on the second read. In the larger experiment, we profiled 259,155 cells to an average depth of 46,523 reads per cell.

6. Lentivirus Vector Construction and Particle Production

To test whether transcription factors (TFs) improve late-stage reprogramming efficiency, we generated lentiviral constructs for the top candidates Zfp42, and Obox6. cDNAs for these factors were ordered from Origene (Zfp42-MG203929, and Obox6-MR215428) and cloned into the FUW Tet-On vector (Addgene, Plasmid #20323) using the Gibson Assembly (NEB, E2611S). Briefly, the cDNA for each TF was amplified and cloned into the backbone generated by removing Oct4 from the FUW-Teto-Oct4 vector. All vectors were verified by Sanger sequencing analysis. For lentivirus production, HEK293T cells were plated at a density of 2.6×10⁶ cells/well in a 10 cm dish. The cells were transfected with the lentiviral packaging vector and a TF-expressing vector at 70-80% growth confluency using the Fugene HD reagent (Promega E2311), according to the manufacturer's protocols. At 48 hours after transfection, the viral supernatant was collected, filtered and stored at −80° C. for future use.

7. Reprogramming Efficiency of Secondary MEFS Together with Individual TFs

We sought to determine the ability of the candidate TFs to augment reprogramming efficiency in secondary MEFs; the use of secondary MEFs for reprogramming overcomes limitations associated with random lentiviral integration events at variable genomic locations. Briefly, secondary MEFs were plated at a concentration of 20,000 cells per well of a 6-well plate. Cells were infected with virus containing Zfp42, Obox6, or an empty vector and maintained in reprogramming medium as described above. At day 8 after induction, cells were switched to either Phase-2(2i) or Phase-2(serum). On day 16, reprogramming efficiency was quantified by measuring the levels of the EGFP reporter driven by the endogenous Oct4 promoter. FACS analyses was performed using the Beckman Coulter CytoFLEX S, and the percentage of Oct4-EGFP cells was determined. Triplicates were used to determine average and standard deviation.

8. Reprogramming Efficiency of Primary MEFS with Individual TFs and OKSM

We also independently tested the performance of TFs in primary MEFs. To this end, lentiviral particles were generated from four distinct FUW-Teto vectors, containing Oct4, Sox2, Klf4, and Myc, previously developed in the Jaenisch lab. MEFs from the background strain B6.Cg-Gt(ROSA)26Sor^{tm1(rtTA*M2)Jae}/J_B6; 129S4-Pou5f1^{tm2Jae}/J were infected with these lentiviral particles, together with a lentivirus expressing tetracycline-inducible Zfp42, Obox6 or no insert. Infected cells were then induced with 2 μg/mL doxycycline in ESC reprogramming medium (day 0). At day 8 after induction, cells were switched to either Phase-2(2i) or Phase-2(serum). On day 16, the number of Oct4-EGFP colonies were counted using a fluorescence microscope. Triplicates for each condition used to determine average values and standard deviation.

IV. Preparation of Expression Matrices

To compute an expression matrix from scRNA-Seq data, we aligned sequenced reads to obtain a matrix U of UMI counts, with a row for each gene and a column for each cell. To reduce variation due to fluctuations in the total number of transcripts per cell, we divide the UMI vector for each cell by the total number of transcripts in that cell. Thus we define the expression matrix E in terms of the UMI matrix U via:

$$E = \frac{{U}_{i j}}{{\sum }_{i = 1}^{G} {U}_{i j}} \times 1 {0}^{4} .$$

In our subsequent analysis, we make use of two variance-stabilizing transforms of the expression matrix E. In particular, we define
1. \tilde{E} to be the log-normalized expression matrix. The entries of \tilde{E} are obtained via

\tilde{E}=log(Eᵢⱼ+1)
2. Ē to be the truncated expression matrix. The entries of Ē are obtained by capping the entries of \tilde{E} at the 99.5% quantile.

When we refer to an expression profile, by default we refer to a column of \tilde{E} unless otherwise specified.

1. Aligning Reads

The 98 bp reads were aligned to the UCSC mm10 transcriptome, and a matrix of UMI counts was obtained using Cellranger from the 10× Genomics pipeline (v2.0.0) with default parameters (https://support.10×genomics.com/single-cell-gene-expression/software/pipelines/latest/installation). Quality control metrics about barcoding and sequencing such as the estimated number of cells per collection and the median number of genes detected across cells are summarized in Table 14. To estimate expression of exogenous OKSM factors from OKSM cassette, we extracted RBGpA sequence (839 bp) from the OKSM cassette FASTA file, and generated a reference using the mkref function from the Cellranger pipeline.

2. Downsampling and Filtering Expression Matrix

The expression matrix was downsampled to 15,000 UMIs per cell. Cells with less than 2000 UMIs per cell in total and all genes that were expressed in less than 50 cells were discarded, leaving 251,203 cells and G=19,089 genes for further analysis. The elements of expression matrix were normalized by dividing UMI count by the total UMI counts per cell and multiplied by 10,000 i.e. expression level is reported as transcripts per 10,000 reads.

3. Selecting Variable Genes

We used the function MeanVarPlot from the Seurat package (v2.1.0) (Satija et al., 2015) to select 1479 variable genes. First, we divided genes into 20 bins based on their average expression levels across all cells. Second, we computed Fano factor of gene expression in each bin and then z-scored. The Fano factor, defined as the variance divided by the mean, was a measure of dispersion. Finally, by thresholding the z-scored dispersion at 1.0, we obtained a set of 1479 variable genes. After selecting variable genes, we created a variable gene expression matrix by renormalizing as described above.

V. Visualization: Force-Directed Layout Embedding

In this section we introduced our two dimensional visualization technique based on force-directed layout embedding (FLE) (Bastian et al., 2009; Jacomy et al., 2014). FLE was large-scale graph visualization tool which simulated the evolution of a physical system in which connected nodes experience attractive forces, but unconnected nodes experience repulsive forces. It better captured global structures than tSNE. Initial FLE algorithms used simple electrostatic and spring forces, but modern FLE algorithms allowed for more elaborate interactions that could depend on the degree of nodes or included gravity terms that attracted all nodes to the center (this was especially important for disconnected graphs, which would otherwise fly apart). Starting from a random initial position of vertices, the network of nodes evolved in such a manner that at any iteration a new position of vertices was computed from the net forces acting on them.

We applied FLE to visualize the nearest neighbor graph generated from our data.

Implementation: Our visualization took as input the expression matrix of highly-variable genes, selected as described in the previous section of the STAR Methods. First, we reduced to 100 dimensions by computing a 100 dimensional diffusion component embedding of the dataset using SCANPY (v0.2.8) with default parameters. Second, for each cell we computed its 20 nearest neighbors in 100-dimensional diffusion component space to produce a nearest neighbor graph. For this step, we used the approximate k-NN algorithm Annoy from the R package RCPPANNOY (v0.0.10). Finally, we computed the force-directed layout on the k-NN graph using the ForceAtlas2 algorithm (Jacomy et al., 2014) from the Gephi Toolkit (v0.9.2) (Bastian et al., 2009).

VI. Creating Gene Signatures and Cell Sets

1. Gene Signatures

We then constructed curated gene signatures from various databases of gene signatures. Given a set of genes, we scored cells based on their gene expression. In particular, for a given cell we computed the z-score for each gene in the set. We then truncated these z-scores at 5 or −5, and defined the signature of the cell to be the mean z-score over all genes in the gene set.

The table below summarizes the sources from which we obtained signatures. In two cases (neural identity and epithelial identity), we constructed signatures manually using marker genes. A pluripotency gene signature was determined in this work using the pilot dataset. We performed differential gene expression analysis between two groups of cells: mature iPSCs and cells along the time course D0 to D16 and took the top 100 genes with increased expression in mature iPSCs. A proliferation gene signature was obtained by combining genes expressed at G1/S and G2/M phases.

In several places, we also computed gene signatures based on co-expression with a given gene of interest. For instance, in the stromal region we noticed several genes (Cxcl12, Ifitm1, and Matn4) with expression patterns that were distinct from a signature of long-term cultured MEFs (FIG. 31D). For each gene, we computed a co-expression signature by finding the set of genes with expression levels in stromal cells that were >15% correlated with the gene of interest. We found that these gene signatures were significantly overlapping (p-value<0.01, hypergeometric test) with signatures of stromal cells in neonatal muscle and neonatal skin in the Mouse Cell Atlas. Similarly, in the neural region we derived signatures of genes co-expressed with Gad1 and with Slc17a6 (FIG. 33C). These signatures significantly overlapped signatures of inhibitory and excitatory neurons, respectively, derived from the Allen Brain Atlas.

| Gene Signature | Source |
|---|---|
| MEF identity | (Chen et al., 2013; Han et al., 2018; |
| | Lattin et al., 2008) |
| Pluripotency | This work. |
| Proliferation | (Tirosh et al., 2016) |
| ER stress | GO:0034976, Biological Process Ontology |
| Epithelial identity | This work. |
| | Marker genes: (Li et al., 2010; Takaishi |
| | et al., 2016; Whiteman et al., 2014) |
| ECM rearrangement | GO:0030198, Biological Process Ontology |
| Apoptosis | Hallmark P53 Pathway, MSigDB |
| Senescence | (Coppé et al., 2010) |
| Neural identity | This work. |
| | Marker gene sources: (Fonseca et al., 2013; |
| | Gouti et al., 2011; Kan et al., 2004; Lazarov |
| | et al., 2010; Sakakibara et al., 2001; Sansom |
| | et al., 2009; Watanabe et al., 2017) |
| Trophoblast | (Han et al., 2018) |
| X reactivation | chromosome X |
| XEN | (Lin et al., 2016) |
| Trophoblast progenitors | (Han et al., 2018) |
| Spiral Artery Trophpblast | (Han et al., 2018) |
| Giant Cells | |
| Oligodendrocyte precursor | (Tasic et al., 2016) |
| cells (OPC) | |
| Astrocytes | (Tasic et al., 2016) |
| Cortical Neurons | (Tasic et al., 2016) |
| RadialGlia-Id3 | (Han et al., 2018) |
| RadialGlia-Gdf10 | (Han et al., 2018) |
| RadialGlia-Neurog2 | (Han et al., 2018) |
| Long-term MEFs | (Han et al., 2018) |
| Embryonic mesenchyme | (Han et al., 2018) |
| Cxcl12 co-expressed | This work. |
| Ifitm1 co-expressed | This work. |
| Matn4 co-expressed | This work. |
| 2,4,8,16,32-cell | (Goolam et al., 2016) |

2. Cell Sets

Using the gene signatures described above, we created coarse cell sets defining the broad regions of the landscape (iPSC, Trophoblast, Neural, Stromal, Epithelial, and MET), and cell subtype sets defining different cell types within a region (stromal, trophoblast, and neural subtypes, along with 2- through 32-cell stages).

To define the coarse cell sets, we first computed a rough partitioning of the landscape by clustering cells using the Louvain method of spectral clustering to obtain 65 cell clusters using k=5 nearest neighbors (FIG. 34A). By examining signature score activity levels over clusters, we grouped several clusters to form cell sets for the iPSC, Stromal and Neuronal regions. Because our densely sampled data did not always segregate into distinct clusters, we defined some additional coarse cell sets by signature scores. We defined the trophoblast cell set to include all cells with Trophoblast signature greater than 0.7. We defined the epithelial cell set to include all cells with epithelial identity signature greater than 0.8, minus all cells included in other cell sets (mostly removing the trophoblasts with epithelial signature). Finally, we defined the MET Region as the ancestors of iPS, Trophoblast, Neural and Epithelial cells. In particular, we computed the top ancestors of each major cell set, then merged these cell sets and removed the cells in each major cell set.

Within the Stromal, Trophoblast, Neural and iPSC cell sets, we then conducted more sensitive statistical tests for cell subtype signatures. We did this by calculating empirical p-values for the subtype signature score for each (region-specific) subtype in each cell. In each of 100,000 permutation trials, we randomly and independently shuffled the expression levels of each gene across the cells within a region. In each cell, we then computed signature scores in the permuted data, and generated p-values by determining the frequency at which the permuted score was greater than the original score. While the results shown in figures and discussed in the main text were based on shuffling genes across cells, we similarly permuted the expression levels within each cell, and found consistent results. Finally, we controlled for multiple hypothesis testing by calculating FDR q-values, and used a threshold FDR of 10% to define cell subtype sets.

VII. Estimating Growth and Death Rates and Computing Transport Maps

1. Initial Estimate of Growth Rates

We formed an initial estimate of the relative growth rate as the expectation of a birth-death process on gene expression space with birth-rate β(x) and death rate δ(x) defined in terms of expression levels of genes involved in cell proliferation and apoptosis. Multi-state birth-death processes had been used before to model growth, death, and transitions in iPS reprogramming (Liu et al., 2016). A birth-death process was a classical model for how the number of individuals in a population could vary over time. The model was specified in terms of a birth rate β and death rate δ: During a time interval Δt, the probability of a birth was βΔt and the probability of a death was δΔt. The doubling time for a birth death process was defined as follows. Starting with N(0)=n, the time τ it would take to get to an expected population size of [symbol] N(t)=2n is

$$τ = \frac{ln 2}{β - δ}$$

The half-life could be computed in a similar way. We applied a sigmoid function to transform the proliferation score into a birth rate. The sigmoid function smoothly interpolated between maximal and minimal birth rates. We specified the maximal birth rate to be β_{MAX}=1.7. Therefore, the fastest cell doubling time is

$$\frac{ln 2}{1.7} \approx 0.41 days \approx 9.6 hours ,$$

by the doubling time equation above. We defined the minimal birth rate as β_{MIN}=0.3. Therefore the slowest cell doubling time is

$$\frac{ln 2}{0.3} = 2.3 days = 55 hours .$$

Similarly, we transformed the apoptosis signature into an estimate of cellular death rates by applying a sigmoid function to smoothly interpolate between minimal and maximal allowed death rates. We defined the minimal death rate parameter to be δ_{MIN}=0.3, and the maximal death rate parameter as δ_{MAX}=1.7. By the calculations above, these correspond to half-lifes of 55 and 9.6 hours respectively.

2. Learning Growth Rates and Computing Transport Maps

Using the growth rates defined in the previous section as an initial estimate, we computed transport maps and automatically improved these growth rates using the Waddington-OT software package (see Section Computing transport maps). For the cost function, we used squared Euclidean distance in 30 dimensional local PCA space computed on the variable gene data from the relevant pair of time points. We used the following parameter settings:

ϵ=0.05,λ₁=1,λ_{z}=50,growth_iters=3.

The parameters λ₁ and λ₂ control the degree to which the row-sums and column-sums were unbalanced. A larger value of λ₁ induced a greater correlation between the input and output growth rates. The Waddington-OT package iterated the procedure of computing transport maps based on input growth rates, and then using the output growth rates as new input growth rates to recompute transport maps. We ran this for growth_iters=3 total iterations.

This gave us a set of transport maps between each pair of time points, which could be used to estimate the temporal coupling. From this estimate of the temporal coupling, we computed ancestor and descendant distributions to each of the major cell sets defined in the previous section.

VIII. Regulatory Analysis

We performed regulatory analysis to identify modules of transcription factors regulating modules of genes with our global regulatory model from the Waddington-OT software package, described in Section Learning gene regulatory models. The optimization began by specifying the number of gene modules, and establishing an initial estimate for each. We used spectral clustering to initialize the modules: genes were clustered into 50 sets, with one module corresponding to each set, and weights set to 0 for genes outside the set, and 1 for genes within the set.

We then specified a time lag between TF and gene module expression. In order to test for potential regulatory interactions on different time scales, we computed global regulatory models with three time lags: 6 hrs, 48 hrs, and 96 hrs. This allowed us to identify factors that were predictive several days in advance—for instance, Nanog is a very early predictor of pluripotency and was found to be associated with a pluripotency associated gene expression module in the 96 hour model—as well as those predictive on shorter time scales—for instance, we TFs that were predictive of neural-associated expression modules in the 6 and 48 hour models, but did not find such predictive TFs in the 96 hour model.

Finally, we set regularization and stochastic block size parameters. Default values available in the code online were used in this study. Briefly, regularization parameters were tuned on small training datasets to enforce sparsity (11 penalties) and reduce model complexity (12 penalty) while still achieving a good fit (>60% correlation between predicted and observed expression) in training data. These parameters may be specifically tuned in new datasets. The stochastic block size and number of epochs were set according to available hardware resources.

IX. Validation by Geodesic Interpolation

We validated Waddington-OT by demonstrating that we could accurately interpolate the distribution of cells at held out time points. We applied geodesic interpolation (described in Waddington-OT: Concepts and Implementation) to our reprogramming data to predict the distribution of cells at each time point, using only the data from the previous and next time points. In other words, we sought to predict the distribution Pₜ_{₂} at time t₂ from the distributions at neighboring time points: Pₜ_{₁} and Pₜ_{₃} (FIGS. 24H, 30D). To determine a baseline for performance, we examined the distance between the two different batches of the held-out distribution (FIGS. 24H, 30D).

To compute the optimal transport coupling from Pₜ_{₁} to Pₜ_{₃}, we used the Waddington-OT package with default parameters. For the cost function we computed 30 dimensional local PCA coordinates using only the points from time t₁ and t₃. We then embedded the data from time t₂ into the 30 dimensional local PCA space which was computed using only the data from time t₁ and t₃. Finally, we used Wasserstein-2 distance to compute distance between point clouds.

X. Paracrine Signaling

To characterize potential cell-cell interactions between contemporaneous cells during reprogramming, we first collected a list of ligands and receptors found in the GO database. The set of ligands (415 genes) was a union of three gene sets from the following GO terms:
1) cytokine activity (GO:0005125),
2) growth factor activity (GO:0008083), and
3) hormone activity (GO:0005179).

The set of receptors (2335 genes) was defined by the GO term receptor activity (GO:0004872). Next, we used a curated database of mouse protein-protein interactions (Mertins et al., 2017) and identified 580 potential ligand-receptor pairs.

First, we defined an interaction score I_{A;B;X;Y;t} as the product of (1) the fraction of cells (F_{A;X;t}) in cell-set A expressing ligand X at time t and (2) the fraction of cells (F_{B;Y;t}) in cell-set B expressing the cognate receptor Y at time t. We define the aggregate interaction score I_{A;B;t} as a sum of the individual interaction scores across all pairs:

$${I}_{A ; B ; t} = {\sum }_{All X \cdot Y pairs} {J}_{A ; B ; X ; Y ; t} = {\sum }_{All X \cdot Y pairs} {F}_{A ; X ; t} {F}_{B ; Y ; t}$$

We depicted the aggregate interaction scores for all combinations of cell clusters in FIGS. 28B, 34B.

Second, we sought to explore individual ligand-receptor pairs at a given day and condition between cell ancestors of interest. For this purpose we defined the interaction score I_{A;B;X;Y;t} as the product of (1) the average expression of the ligand X in ancestors at time t of a cell set A and (2) the average expression of the cognate receptor Y in ancestors at time t of a cell set B. Values of the interaction scores I_{A;B;X;Y;t} are high for ubiquitously expressed ligands and receptors at a given day and may be nonspecific to a pair of cell ancestors of interest. Thus, we used permutations to generate an empirical null distribution of interaction scores. In each of the 10,000 permutations, we randomly shuffled the labels of cells and calculated the interaction score I^{s}_{A;B;X;Y;t}. We then standardized each ligand-receptor interaction score by taking the distance between the interaction score I_{A;B;X;Y;t} and the mean interaction score in units of standard deviations from the permuted data

((I_{A;B;X;Y;t}−mean(I^{s}_{A;B;X;Y;t}))/sd(I^{s}_{A;B;X;Y;t})).

We depicted examples of standardized interaction scores ranked by their values in FIGS. 28C-28E and 34C-34E. Replacement of the average expression of the ligand with the total expression of the ligand in the calculation of the standardized interaction score did not affect the results.

XI. Classification of Differential Genes Along the Trajectory to iPSCs

To identify differential genes along the successful trajectory to iPSCs we computed the average expression (TPM) of all 19,089 genes in ancestors of iPSCs. The average expression values were log 2 transformed and we filtered out genes for which the difference between maximal and minimal expression value between day 0 and day 18 was less than 1, leaving 2311 genes for further analysis. The genes were classified into 15 groups by k-means clustering as implemented in the R package stats. To identify the number of clusters we applied a gap statistic (Tibshirani et al. 2001) using the function clusGap from R package cluster v2.0.6.

We performed functional enrichment analysis on the identified gene clusters using the findGO.pl program from the HOMER suite (Hypergeometric Optimization of Motif Enrichment, v4.9.1) (Heinz et al. 2010) with Benjamini and Hochberg FDR correction for multiple hypothesis testing (retaining terms at FDR<0.05). All genes that passed quality-control filters were used as a background set.

XII. Identifying Large Chromosomal Aberrations

We have previously developed methods to identify copy number variations (CNVs) in scRNA-Seq data from tumor samples (Patel et al., 2014; Tirosh et al., 2016). That analysis differed from our current study in two key aspects: (1) the data were based on full length scRNA-seq (SMART-Seq2), and sequenced to greater depth in each cell, and (2) there we could rely on the clonal expansion of CNVs to make it easier to identify recurring chromosomal aberrations.

We performed three types of analysis to detect aberrant expression in large chromosomal regions. First, we searched cells with significant up- or down-regulation at the level of entire chromosomes. Second, we ran a coarse analysis to identify cells with significant net aberrant expression across windows spanning 25 broadly-expressed genes. Focusing on regions that were enriched for cells with significant aberrations found by this coarse filter, we then performed a more sensitive test to compute the significance of aberrations in each window in each cell.

Empirical p-values and false discovery rates (FDRs) for both analyses were computed by randomly permuting the arrangement of genes in the genome, as described below. Permutations for both types of analysis were done as follows. In each of 100,000 permutations we randomly shuffled the labels of genes in the entire dataset, while preserving the genomic coordinates of genes (with each position having a new label each time) and the expression levels in each cell (so that each cell has the same expression values, but with new labels). We then computed either whole chromosome or subchromosomal aberration scores for each cell.

To identify whole-chromosome aberrations scores in each cell, we began by calculating the sum of expression levels in 25Mbp sliding windows along each chromosome, with each window sliding 1Mbp so that it overlapped the previous window by 24Mbp. For each window in each cell, we then calculated the Z-score of the net expression, relative to the same window in all other cells. We then counted the fraction of windows on each chromosome with an absolute value Z-score>2. This fraction served as the whole-chromosome aberration score for each chromosome in each cell. To assign a p-value to the whole-chromosome score for cell(i) chromosome(j), we calculated the empirical probability that the score for cell(i) chromosome(j) in the randomly permuted data was at least as large as the score in the original data.

Subchromosomal aberration scores were computed as follows. We began by identifying the 20% of genes with the most uniform expression across the entire dataset. This was done by calculating the Shannon Diversity e^{-Σ}^{_{g}}^{E}^{_{gc}}^{lnE}^{_{gc}} for each gene g (where E_{gc} was the expression matrix as defined above in Preparation of expression matrices), and taking the 20% of genes with the largest values. Using these genes, we subset the expression matrix and renormalized by TPM, and then computed in each cell the sum of expression in sliding windows of 25 consecutive genes, with each window sliding by one gene and overlapping the previous window (on the same chromosome) by 24 genes. In each window, we calculated the Z-score relative to all cells at day 0. The net (coarse filter) subchromosomal aberration score for a cell was calculated as the 12-norm of the Z-scores across all windows. To assign a p-value to the subchromosomal aberration score for cell(i), we calculated the empirical probability that the score for cell(i) in the randomly permuted data was at least as large as the score in the original data.

Finally, to identify the specific region(s) of genomic aberrations in each cell, we conducted a more sensitive test using just the cells in the stromal and trophoblast regions. Again using 25 housekeeping gene windows, we computed the average z-score of gene expression for genes in each window in each cell. We then compared the scores in all windows in all cells to similar scores computed for each cell in 100,000 random permutation trials, and then assigned p-values based on the frequency of extremely high (gain) or low (loss) expression values.

For each of the aberration scores and associated p-values described above, we controlled for multiple hypothesis testing by calculating FDR q-values, using a false discovery threshold of 10%.

Quantification and Statistical Analysis

I. Analyzing the Stability of Optimal Transport

To test the stability of our optimal transport analysis to perturbations of the data and parameter settings, we downsampled the number of cells at each time point, downsampled the number of reads in each cell, perturbed our initial estimates for cellular growth and death rates, and perturbed the parameters for entropic regularization and unbalanced transport. We found that our geodesic interpolation results are stable to a wide range of perturbations, summarized in the following table:

| Number | Number | Max | Min | Max | Min | Entropy | Unbalanced |
|---|---|---|---|---|---|---|---|
| of cells | of UMIs | Growth | Growth | Death | Death | regularization | transport |
| per batch | Per cell | β_{MAX} | β_{MIN} | δ_{MAX} | δ_{MIN} | ∈ | λ |
| Down | Down | 33 hrs | None | 33 hrs | None | 5 × 10⁻⁵ | 0.1 |
| to: | to: | to | to | to | to | to | to |
| 200 | 1000 | 5.5 hrs | 9.5 hrs | 5.5 hrs | 9.5 hrs | 0.5 | 32 |

To generate this table, we ran geodesic interpolation with all but one of these settings fixed to default values. The default parameter values that we used were:
ϵ=0.05, λ₁=1, λ₂=50, β_{MAX}=1.7, δ_{MAX}=1.7, β_{MIN}=0.3, δ_{MIN}=0.3.

Moreover, by default we used all reads per cell and all cells per batch.

II. Performance of Other Methods

1. Monocle2

Monocle2 fitted the data into a graph without using prior information of the number of potential fates (Qiu et al., 2017).

We ran Monocle2 (v2.8.0) with default parameters on a subset of our dataset containing 1,000 cells per time point. Running on our full dataset would require more RAM than we had access to.

In our data, Monocle2 failed to distinguish iPS, neuronal-like, and trophoblast-like cells as distinct destinations (FIG. 35A-35B). It put together day 18 stromal cells and day 0 MEFs at the root of the tree, and placed iPS, neural-like and trophoblast-like cells on a different branch from cells in the MET Region. Moreover, because the program could incorporate temporal information, it returned a trajectory that was inconsistent with the measured temporal progression. The output of the program implied that day 0 MEF cells gave rise to day 18 stromal cells, which in turn gave rise to everything else.

2. URD

URD identified trajectories from a user-specified root to a set of user-specified tips by performing random walks according to a Markov diffusion kernel.

We ran URD (v1.0) with default parameters on a subset of our dataset containing 1,000 cells per time point. Running on our full dataset would require more RAM than we had access to.

In our data, URD predicted that all fates diverge extremely early, with stromal cells diverging from other cells soon after day 0; trophoblast-like cells diverging from neural-like and iPS cells as early as day 1; and neural-like and iPS cells diverging at day 2 (FIGS. 35A-35B). Additionally, URD failed to assign over half (51%) of the cells to any trajectory.

Comparing the two branches for iPS and neural (FIGS. 35A-35B—segments 6 and 7) revealed no distinctive pattern between the supposedly divergent trajectories from day 3-8. The divergent trajectories appeared to be an artifact of the fact that the method requires a distinct branch point.

Moreover, because the method did not incorporate growth rates, the transitions to iPS and Neural come disproportionately from stromal cells.

III. Pilot study

In our pilot study, we collected 65,000 expression profiles over 16 days at 10 distinct time points (and 9 in serum). We compared results from the larger study to the pilot study in FIGS. 30A-30G, where we showed trends in expression along trajectories to each major cell set: iPSCs, Neural-like, Trophoblast-like (placenta-like in pilot), and Stromal. We found that the expression trends were reasonably similar. Moreover, by comparing the ancestor divergence plots for the two studies, we found that in both studies the stromal population gradually diverged early in the time course and there was a sharp divergence of iPSC from Neural and Trophoblast just after removal of Dox at day 8.

Data and Software Availability

We have uploaded our data to NCBI Gene Expression Omnibus. The identification numbers are:

| | |
|---|---|
| Single cell RNA-seq raw data (pilot study) | GSE106340 |
| Single cell RNA-seq raw data | GSE115943 |

Our software package is available on GitHub: https://github.com/broadinstitute/wot

S

### REFERENCE CITED

1. C. H. Waddington, How animals develop. (New York, 1936).
2. C. H. Waddington, The strategy of the genes; a discussion of some aspects of theoretical biology. (London, Allen & Unwin [1957], 1957).
3. E. Z. Macosko et al., Highly parallel genome-wide expression profiling of individual cells using nanoliter droplets. Cell 161, 1202-1214 (2015).
4. A. M. Klein et al., Droplet barcoding for single-cell transcriptomics applied to embryonic stem cells. Cell 161, 1187-1201 (2015).
5. G. X. Zheng et al., Massively parallel digital transcriptional profiling of single cells. Nature communications 8, 14049 (2017).
6. A. Tanay, A. Regev, Scaling single-cell genomics from phenomenology to mechanism. Nature 541, 331-338 (2017).
7. A. Wagner, A. Regev, N. Yosef, Revealing the vectors of cellular identity with single-cell genomics. Nat Biotech 34, 1145-1160 (2016).
8. S. C. Bendall et al., Single-cell trajectory detection uncovers progression and regulatory coordination in human B cell development. Cell 157, 714-725 (2014).
9. C. Trapnell et al., The dynamics and regulators of cell fate decisions are revealed by pseudotemporal ordering of single cells. Nature biotechnology 32, 381-386 (2014).
10. M. Setty et al., Wishbone identifies bifurcating developmental trajectories from single-cell data. Nature biotechnology 34, 637-645 (2016).
11. E. Marco et al., Bifurcation analysis of single-cell gene expression data reveals epigenetic landscape. Proceedings of the National Academy of Sciences of the United States of America 111, E5643-5650 (2014).
12. J. M. Polo et al., A molecular roadmap of reprogramming somatic cells into iPS cells. Cell 151, 1617-1632 (2012).
13. Y. Buganim et al., Single-cell expression analyses during cellular reprogramming reveal an early stochastic and a late hierarchic phase. Cell 150, 1209-1222 (2012).
14. S. M. Hussein et al., Genome-wide characterization of the routes to pluripotency. Nature 516, 198 (2014).
15. P. D. Tonge et al., Divergent reprogramming routes lead to alternative stem-cell states. Nature 516, 192-197 (2014).
16. J. O'Malley et al., High resolution analysis with novel cell-surface markers identifies routes to iPS cells. Nature 499, 88 (2013).
17. X. Qiu et al., Reversed graph embedding resolves complex single-cell developmental trajectories. bioRxiv, 110668 (2017).
18. S. C. Bendall et al., Single-cell trajectory detection uncovers progression and regulatory coordination in human B cell development. Cell 157, 714-725 (2014).
19. R. Rostom, V. Svensson, S. Teichmann, G. Kar, Computational approaches for interpreting scRNA-seq data. FEBS letters, (2017).
20. L. Haghverdi, F. Buettner, F. J. Theis, Diffusion maps for high-dimensional single-cell analysis of differentiation data. Bioinformatics 31, 2989-2998 (2015).
21. L. Haghverdi, M. Buttner, F. A. Wolf, F. Buettner, F. J. Theis, Diffusion pseudotime robustly reconstructs lineage branching. Nat Meth 13, 845-848 (2016).
22. K. Campbell, C. Yau, Ouija: Incorporating prior knowledge in single-cell trajectory learning using Bayesian nonlinear factor analysis. bioRxiv, (2016).
23. R. Cannoodt et al., SCORPIUS improves trajectory inference and identifies novel modules in dendritic cell development. bioRxiv, (2016).
24. J. D. Welch, A. J. Hartemink, J. F. Prins, SLICER: inferring branched, nonlinear cellular trajectories from single cell RNA-seq data. Genome Biology 17, 106 (2016).
25. K. Street et al., Slingshot: Cell lineage and pseudotime inference for single-cell transcriptomics. bioRxiv, (2017).
26. H. Matsumoto, H. Kiryu, SCOUP: a probabilistic model based on the Ornstein-Uhlenbeck process to analyze single-cell expression data during differentiation. BMC Bioinformatics 17, 232 (2016).
27. S. Rashid, D. N. Kotton, Z. Bar-Joseph, TASIC: determining branching models from time series single cell data. Bioinformatics 33, 2504-2512 (2017).
28. M. Zwiessele, N. D. Lawrence, Topslam: Waddington Landscape Recovery for Single Cell Experiments. bioRxiv, (2016).
29. C. Weinreb, S. Wolock, B. K. Tusi, M. Socolovsky, A. M. Klein, Fundamental limits on dynamic inference from single cell snapshots. bioRxiv, (2017).
30. C. Villani, Optimal transport: old and new. (Springer Science & Business Media, 2008), vol. 338.
31. M. Cuturi, in Advances in neural information processing systems. (2013), pp. 2292-2300.
32. L. Chizat, G. Peyre, B. Schmitzer, F.-X. Vialard, Scaling algorithms for unbalanced transport problems. arXiv preprint arXiv:1607.05816, (2016).
33. J. H. Levine et al., Data-Driven Phenotypic Dissection of AML Reveals Progenitor-like Cells that Correlate with Prognosis. Cell 162, 184-197 (2015).
34. K. Shekhar et al., Comprehensive Classification of Retinal Bipolar Neurons by Single-Cell Transcriptomics. Cell 166, 1308-1323.e1330 (2016).
35. R. R. Coifman et al., Geometric diffusions as a tool for harmonic analysis and structure definition of data: Diffusion maps. Proceedings of the National Academy of Sciences of the United States of America 102, 7426-7431 (2005).
36. M. Jacomy, T. Venturini, S. Heymann, M. Bastian, ForceAtlas2, a continuous graph layout algorithm for handy network visualization designed for the Gephi software. PloS one 9, e98679 (2014).
37. E. R. Zunder, E. Lujan, Y. Goltsev, M. Wernig, G. P. Nolan, A continuous molecular roadmap to iPSC reprogramming through progression analysis of single-cell mass cytometry. Cell Stem Cell 16, 323-337 (2015).
38. C. Weinreb, S. Wolock, A. Klein, SPRING: a kinetic interface for visualizing high dimensional single-cell expression data. bioRxiv, (2016).
39. K. Takahashi, S. Yamanaka, Induction of pluripotent stem cells from mouse embryonic and adult fibroblast cultures by defined factors. cell 126, 663-676 (2006).
40. J. Yu et al., Induced pluripotent stem cell lines derived from human somatic cells. Science 318, 1917-1920 (2007).
41. J. Shu et al., Induction of pluripotency in mouse somatic cells with lineage specifiers. Cell 153, 963-975 (2013).
42. P. Hou et al., Pluripotent Stem Cells Induced from Mouse Somatic Cells by Small-Molecule Compounds. Science 341, 651-654 (2013).
43. D. H. Kim et al., Single-cell transcriptome analysis reveals dynamic changes in lncRNA expression during reprogramming. Cell stem cell 16, 88-101 (2015).
44. A. Parenti, M. A. Halbisen, K. Wang, K. Latham, A. Ralston, OSKM induce extraembryonic endoderm stem cells in parallel to induced pluripotent stem cells. Stem cell reports 6, 447-455 (2016).
45. T. S. Mikkelsen et al., Dissecting direct reprogramming through integrative genomic analysis. Nature 454, 49 (2008).
46. M. Stadtfeld, N. Maherali, M. Borkent, K. Hochedlinger, A reprogrammable mouse strain from gene-targeted embryonic stem cells. Nature methods 7, 53-55 (2010).
47. Z. D. Smith, I. Nachman, A. Regev, A. Meissner, Dynamic single-cell imaging of direct reprogramming reveals an early specifying event. Nat Biotechnol 28, 521-526 (2010).
48. J. Pei, N. V. Grishin, Unexpected diversity in Shisa-like proteins suggests the importance of their roles as transmembrane adaptors. Cellular signalling 24, 758-769 (2012).
49. M. Meyyappan, H. Wong, C. Hull, K. T. Riabowol, Increased expression of cyclin D2 during multiple states of growth arrest in primary and established cells. Molecular and cellular biology 18, 3163-3172 (1998).
50. J.-P. Coppe, P.-Y. Desprez, A. Krtolica, J. Campisi, The senescence-associated secretory phenotype: the dark side of tumor suppression. Annual Review of Pathological Mechanical Disease 5, 99-118 (2010).
51. L. Mosteiro et al., Tissue damage and senescence provide critical signals for cellular reprogramming in vivo. Science 354, aaf4445 (2016).
52. Q.-L. Ying et al., The ground state of embryonic stem cell self-renewal. Nature 453, 519 (2008).
53. I. Tirosh et al., Single-cell RNA-seq supports a developmental hierarchy in human oligodendroglioma. Nature 539, 309-313 (2016).
54. S. C. Andrews et al., Cdknlc (p57 Kip2) is the major regulator of embryonic growth within its imprinted domain on mouse distal chromosome 7. BMC Developmental Biology 7, 53 (2007).
55. N. Barker et al., Identification of stem cells in small intestine and colon by marker gene Lgr5. Nature 449, 1003-1007 (2007).
56. G. C. Elson et al., CLF associates with CLC to form a functional heteromeric ligand for the CNTF receptor complex. Nature neuroscience 3, 867 (2000).
57. A. Fowden, C. Sibley, W. Reik, M. Constancia, Imprinted genes, placental development and fetal growth. Hormone Research in Paediatrics 65, 50-58 (2006).
58. A. Ralston et al., Gata3 regulates trophoblast development downstream of Tead4 and in parallel to Cdx2. Development 137, 395-403 (2010).
59. G. Burton, H.-W. Yung, T. Cindrova-Davies, D. Charnock-Jones, Placental endoplasmic reticulum stress and oxidative stress in the pathophysiology of unexplained intrauterine growth restriction and early onset preeclampsia. Placenta 30, 43-48 (2009).
60. V. Pasque et al., X chromosome reactivation dynamics reveal stages of reprogramming to pluripotency. Cell 159, 1681-1697 (2014).
61. K. Tomoda et al., Derivation conditions impact X-inactivation status in female human induced pluripotent stem cells. Cell stem cell 11, 91-99 (2012).
62. Q. Bai et al., Dissecting the first transcriptional divergence during human embryonic development. Stem Cell Reviews and Reports 8, 150-162 (2012).
63. A.-H. Monsoro-Burq, E. Wang, R. Harland, Msx1 and Pax3 cooperate to mediate FGF8 and WNT signals during Xenopus neural crest induction. Developmental cell 8, 167-178 (2005).
64. L. Pevny, M. Placzek, SOX genes and neural progenitor identity. Current opinion in neurobiology 15, 7-13 (2005).
65. V. Y. Wang, H. Y. Zoghbi, Genetic regulation of cerebellar development. Nature reviews. Neuroscience 2, 484 (2001).
66. Y. Liu, A. W. Helms, J. E. Johnson, Distinct activities of Msx1 and Msx3 in dorsal neural tube development. Development 131, 1017-1028 (2004).
67. M. Bergsland et al., Sequentially acting Sox transcription factors in neural lineage development. Genes Dev 25, 2453-2464 (2011).
68. K. Achim et al., The role of Tal2 and Tal1 in the differentiation of midbrain GABAergic neuron precursors. Biology open 2, 990-997 (2013).
69. A. Domanskyi, H. Alter, M. A. Vogt, P. Gass, I. A. Vinnikov, Transcription factors Foxa1 and Foxa2 are required for adult dopamine neurons maintenance. Frontiers in cellular neuroscience 8, 275 (2014).
70. K. Takebayashi-Suzuki, A. Kitayama, C. Terasaka-lioka, N. Ueno, A. Suzuki, The forkhead transcription factor FoxB1 regulates the dorsal-ventral and anterior-posterior patterning of the ectoderm during early Xenopus embryogenesis. Developmental biology 360, 11-29 (2011).
71. G. Hu et al., A genome-wide RNAi screen identifies a new transcriptional module required for self-renewal. Genes & development 23, 837-848 (2009).
72. W.-Z. Li et al., Hesx1 enhances pluripotency by working downstream of multiple pluripotency-associated signaling pathways. Biochemical and Biophysical Research Communications 464, 936-942 (2015).
73. W. Shi et al., Regulation of the pluripotency marker Rex-1 by Nanog and Sox2. J Biol Chem 281, 23319-23325 (2006).
74. A. Rajkovic, C. Yan, W. Yan, M. Klysik, M. M. Matzuk, Obox, a Family of Homeobox Genes Preferentially Expressed in Germ Cells. Genomics 79, 711-717 (2002).
[S1) Villani C. Optimal Transport Old and New. Springer; 2008.
[S2] Chizat L, Peyre G, Schmitzer B, Vialard F X. Scaling Algorithms for Unbalanced Transport Problems. Mathematics of Computation. 2017.
[S3] Cuturi M. Sinkhorn Distances: Lightspeed Computation of Optimal Transportation Distances. In: Neural Information Processing Systems (NIPS); 2013.
[S4] https://support. 10×genomics.com/single-cell-gene-expression/software/pipelines/latest/installation.
[S5] Coifman R R, Lafon S, Lee A B, Maggioni M, Nadler B, Warner F, et al. Geometric diffusions as a tool for harmonic analysis and structure definition of data: Diffusion maps. Proc Natl Acad Sci USA. 2005; 102:7426-7431.
[S6] Haghverdi L, Buettner F, Theis F J. Diffusion maps for high-dimensional single-cell analysis of differentiation data. Bioinformatics. 2015; 31:2989-2998.
[S7] Haghverdi L, Buettner M, Wolf F A, Buettner F, Theis F J. Diffusion pseudotyme robustly recon-structs lineage branching. bioRxiv. 2016;p. 041384.
[S8] Angerer P, Haghverdi L, Bu^{e}ttner M, Theis F J, Marr C, Buettner F. destiny: diffusion maps for large-scale single-cell data in R. Bioinformatics. 2015; 32:1241-1243.
[S9] Moignard V, Woodhouse S, Haghverdi L, Lilly A J, Tanaka Y, Wilkinson A C, et al. Decoding the regulatory network of early blood development from single-cell gene expression measurements. Nature Biotechn. 2015; 33:269-276.
[S10] SettyM,TadmorMD,Reich-ZeligerS, Angel O, Salame™, KathailP, et al. Wishbone identifies bifurcating developmental trajectories from single-cell data. Nature Biotechn. 2016; 34:637-645.
[S11] Satija R, Farrell J A, Gennert D, Schier A F, Regev A. Spatial reconstruction of single-cell gene expression data. Nature Biotechn. 2015; 33:495-502.
[S12] HeinzS, BennerC, SpannN, BertolinoE, LinYC, LasloP, etal. Simple combination so flineage-determining transcription factors prime cis-regulatory elements required for macrophage and B cell identities. Mol cell. 2010; 38:576-589.
[S13] Bastian M, Heymann S, Jacomy M, et al. Gephi: an open source software for exploring and manipulating networks. Icwsm. 2009; 8:361-362.
[S14] Jacomy M, Venturini T, Heymann S, Bastian M. ForceAtlas2, a continuous graph layout algo-rithm for handy network visualization designed for the Gephi software. PloS one. 2014; 9:e98679.
[S15] Beygelzimer A, Kakadet S, Langford J, Arya S, Mount D, Li S, et al. Package FNN.
[S16] Zunder E R, Lujan E, Goltsev Y, Wernig M, Nolan G P. A continuous molecular roadmap to iPSC reprogramming through progression analysis of single-cell mass cytometry. Cell Stem Cell. 2015; 16:323-337.
S17 Porpiglia E, Samusik N, Van Ho A T, Cosgrove B D, Mai T, Davis K L, et al. High-resolution myogenic lineage mapping by single-cell mass cytometry. Nature Cell Biol. 2017; 19:558-567.
S18 Samusik N, Good Z, Spitzer M H, Davis K L, Nolan G P. Automated mapping of phenotype space with single-cell data. Nature methods. 2016; 13:493-496.
S19 Blondel V D, Guillaume J L, Lambiotte R, Lefebvre E. Fast unfolding of communities in large networks. J Stat Mech Theor Exp. 2008; 2008:P10008.
S20 Levine J H, Simonds E F, Bendall S C, Davis K L, El-ad D A, Tadmor M D, et al. Data-driven phenotypic dissection of AML reveals progenitor-like cells that correlate with prognosis. Cell. 2015; 162:184-197.
S21 Shekhar K, Lapan S W, Whitney I E, Tran N M, Macosko E Z, Kowalczyk M, et al. Comprehensive classification of retinal bipolar neurons by single-cell transcriptomics. Cell. 2016; 166:1308-1323.
S22 Csardi G, Nepusz T. The igraph software package for complex network research. InterJournal, Complex Systems. 2006; 1695:1-9.
S23 Friedman J, Hastie T, Tibshirani R. Sparse inverse covariance estimation with the graphical lasso. Biostatistics. 2008; 9:432-441.
S24 Rosvall M, Bergstrom C T. Maps of random walks on complex networks reveal community struc-ture. Proc Natl Acad Sci USA. 2008; 105:1118-1123.
S25 Qiu X, Mao Q, Tang Y, Wang L, Chawla R, Pliner H, et al. Reversed graph embedding resolves complex single-cell developmental trajectories. bioRxiv. 2017;p. 110668.
S26 Qiu X, Hill A, Packer J, Lin D, Ma Y A, Trapnell C. Single-cell mRNA quantification and differ-ential analysis with Census. Nature methods. 2017; 14:309-315.
S27 Mao Q, Wang L, Goodison S, Sun Y. Dimensionality reduction via graph structure learning. In: Proceedings of the 21th ACM SIGKDD International Conference on Knowledge Discovery and Data Mining. ACM; 2015. p. 765-774.
S28 Rashid S, Kotton D N, Bar-Joseph Z. TASIC: determining branching models from time series single cell data. Bioinformatics. 2017;p. btx173.
S29 Lattin J E, Schroder K, Su A I, Walker J R, Zhang J, Wiltshire T, et al. Expression analysis of G Protein-Coupled Receptors in mouse macrophages. Immunome Res. 2008; 4:5.
S30 Chen E Y, Tan C M, Kou Y, Duan Q, Wang Z, Meirelles G V, et al. Enrichr: interactive and collaborative HTML5 gene list enrichment analysis tool. BMC Bioinformatics. 2013; 14:128.
S31 Tirosh I, Venteicher A S, Hebert C, Escalante L E, Patel A P, Yizhak K, et al. Single-cell RNA-seq supports a developmental hierarchy in human oligodendroglioma. Nature. 2016; 539:309-313.
S32 Li R, Liang J, Nᵢ S, Zhou T, Qing X, Li H, et al. A mesenchymal-to-epithelial transition initiates and is required for the nuclear reprogramming of mouse fibroblasts. Cell stem cell. 2010; 7:51-63.]
S33 Whiteman E L, Fan S, Harder J L, Walton K D, Liu C J, Soofi A, et al. Crumbs3 is essential for proper epithelial development and viability. Mol Cell Biol. 2014; 34:43-56.
S34 Takaishi M, Tarutani M, Takeda J, Sano S. Mesenchymal to Epithelial Transition Induced by Re-programming Factors Attenuates the Malignancy of Cancer Cells. PloS one. 2016; 11:e0156904.
S35 Hewitt K J, Agarwal R, Morin P J. The claudin gene family: expression in normal and neoplastic tissues. BMC cancer. 2006; 6:186.
S36 Coppe J P, Desprez P Y, Krtolica A, Campisi J. The senescence-associated secretory phenotype: the dark side of tumor suppression. Annu Rev Pathol. 2010; 5:99-118.
S37 da Fonseca E T, Manc,anares ACF, Ambro sio C E, Miglino M A. Review point on neural stem cells and neurogenic areas of the central nervous system. Open J Anim Sci. 2013; 3:242.
S38 Sakakibara Sᵢ, Nakamura Y, Satoh H, Okano H. Rna-binding protein Musashi2: developmentally regulated expression in neural precursor cells and subpopulations of neurons in mammalian CNS. J Neurosci. 2001; 21:8091-8107.
S39 Gouti M, Briscoe J, Gavalas A. Anterior Hox genes interact with components of the neural crest specification network to induce neural crest fates. Stem cells. 2011; 29:858-870.
S40 Watanabe Y, Stanchina L, Lecerf L, Gacem N, Conidi A, Baral V, et al. Differentiation of Mouse Enteric Nervous System Progenitor Cells Is Controlled by Endothelin 3 and Requires Regulation of Ednrb by SOX10 and ZEB2. Gastroenterology. 2017; 152:1139-1150.
S41 Sansom S_{N}, Griffiths D S, Faedo A, Kleinjan D J, Ruan Y, Smith J, et al. The level of the tran-scription factor Pax6 is essential for controlling the balance between neural stem cell self-renewal and neurogenesis. PLoS Genetics. 2009; 5:e1000511.
S42 SKan L, Israsena N, Zhang Z, Hu M, Zhao L R, Jalali A, et al. Sox1 acts through multiple inde-pendent pathways to promote neurogenesis. Dev Biol. 2004; 269:580-594.
S43 Lazarov O, Mattson M P, Peterson D A, Pimplikar S W, van Praag H. When neurogenesis encoun-ters aging and disease. Trends Neurosci. 2010; 33:569-579.
S44 Tibshirani R, Walther G, Hastie T. Estimating the number of clusters in a data set via the gap statistic. J R Stat Soc Series B Stat Methodol. 2001; 63:411-423.
S45 Polo J M, Anderssen E, Walsh R M, Schwarz B A, Nefzger C M, Lim S M, et al. A molecular roadmap of reprogramming somatic cells into iPS cells. Cell. 2012; 151(7):1617-1632.
S46 Mertins P, Przybylski D, Yosef N, Qiao J, Clauser K, Raychowdhury R, et al. An Integrative Framework Reveals Signaling-to-Transcription Events in Toll-like Receptor Signaling. Cell re-ports. 2017; 19(13):2853-2866.
S47 ChoiJ, HuebnerAJ, ClementK, WalshRM, SavolA, LinK, etal. Prolonged Mekl/2suppression impairs the developmental potential of embryonic stem cells. Nature. 2017; 548:219-223.
S48 Parenti A, Halbisen M A, Wang K, Latham K, Ralston A. OSKM induce extraembryonic endo-derm stem cells in parallel to induced pluripotent stem cells. Stem cell reports. 2016; 6(4):447-455.
[S49] Lin J, Khan M, Zapiec B, Mombaerts P. Efficient derivation of extraembryonic endoderm stem cell lines from mouse postimplantation embryos. Scientific reports. 2016; 6.
[S50] Edgar R, Mazor Y, Rinon A, Blumenthal J, Golan Y, Buzhor E, et al. LifeMap Discovery?: the embryonic development, stem cells, and regenerative medicine research portal. PloS one. 2013; 8(7):e66629.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of producing an induced pluripotent stem cell comprising introducing a nucleic acid encoding Obox6 into a target cell to produce an induced pluripotent stem cell.

2. The method of claim 1, further comprising introducing into the target cell at least one nucleic acid encoding a reprogramming factor selected from the group consisting of: Gdf9, Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sal11, Sal14, Esrrb, Nr5a2, Tbx3, and Glis1.

3. The method of claim 1, further comprising introducing into the target cell at least one nucleic acid encoding a reprogramming factor selected from the group consisting of: Oct4, Klf4, Sox2 and Myc.

4. The method of claim 1, wherein the nucleic acid encoding Obox6 is provided in a recombinant vector.

5. The method of claim 4, wherein the vector is a lentivirus vector.

6. The method of claim 2, where the nucleic acid encoding the reprogramming factor is provided in a recombinant vector.

7. The method of claim 1, further comprising a step of culturing the cells in reprogramming medium.

8. The method of claim 1, further comprising a step of culturing the cells in the presence of serum.

9. The method of claim 1, further comprising a step of culturing the cells in the absence of serum.

10. The method of claim 1, wherein the induced pluripotent stem cell expresses at least one of a surface marker selected from the group consisting of: Oct4, SOX2, KLf4, c-MYC, LIN28, Nanog, Glis1, TRA-160/TRA-1-81/TRA-2-54, SSEA1, SSEA4, Sal4, and Esrbb1.

11. The method of claim 1, wherein the target cell is a mammalian cell.

12. The method of claim 1, wherein the target cell is a human cell or a murine cell.

13. The method of claim 1, wherein the target cell is a mouse embryonic fibroblast.

14. The method of claim 1, wherein the target cell is selected from the group consisting of: fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, pancreatic cells, hepatocytes, macrophages, monocytes, mononuclear cells, and gastric cells, including gastric epithelial cells.

15. A method of producing an induced pluripotent stem cell comprising introducing at least one of Obox6, Spic, Zfp42, Sox2, Mybl2, Msc, Nanog, Hesx1 and Esrrb into a target cell to produce an induced pluripotent stem cell.

16. A method of producing an induced pluripotent stem cell comprising introducing at least one of the transcription factors identified in Table 2, Table 3, Table 4, Table 5, and Table 6, into a target cell to produce an induced pluripotent stem cell.

17. A method of increasing the efficiency of production of an induced pluripotent stem cell comprising introducing Obox6 into a target cell to produce an induced pluripotent stem cell.

18. A method of increasing the efficiency of production of an induced pluripotent stem cell comprising introducing at least one of the transcription factors identified in Table 2, Table 3, Table 4, Table 5, and Table 6, into a target cell to produce an induced pluripotent stem cell.

19. An isolated induced pluripotential stem cell produced by the method of claim 1, 15, or 16.

20. A method of treating a subject with a disease comprising administering to the subject a cell produced by differentiation of the induced pluripotent stem cell produced by the method of claim 1, 15, or 16.

21. A composition for producing an induced pluripotent stem cell comprising Obox6 in combination with reprogramming medium.

22. A composition for producing an induced pluripotent stem cell comprising one or more of the factors identified in or one or more of the factors identified in Table 2, Table 3, Table 4, Table 5, and Table 6 in combination with reprogramming medium.

23. Use of Obox6 for production of an induced pluripotent stem cell.

24. Use of a factor identified in or one or more of the factors identified in Table 2, Table 3, Table 4, Table 5, and Table 6 for production of an induced pluripotent stem cell.

25. A method of increasing the efficiency of reprogramming a cell comprising introducing Obox6 into a target cell to produce an induced pluripotent stem cell.

26. A method of increasing the efficiency of reprogramming a cell comprising introducing at least one of the transcription factors identified in Table 2, Table 3, Table 4, Table 5 and Table 6, into a target cell to produce an induced pluripotent stem cell.

27. A computer-implemented method for mapping developmental trajectories of cells, comprising:
generating, using one or more computing devices, optimal transport maps for a set of cells from single cell sequencing data obtained over a defined time course;
determining, using one or more computing devices, cell regulatory models, and optionally identifying local biomarker enrichment, based on at least the generated optimal transport maps;
defining, using the one or more computing devices, gene modules; and
generating, using the one or more computing devices, a visualization of a developmental landscape of the set of cells.

28. The method of claim 27, wherein determining cell regulatory models comprise sampling pairs of cells at a first time and a second time point according to transport probabilities.

29. The method of claim 28, further comprising using the expression levels of transcription factors at the earlier time point to predict non-transcription factor expression at the second time point.

30. The method of claim 27, wherein identifying local biomarker enrichment comprises identifying transcription factors enriched in cells having a defined percentage of descendants in a target cell population.

31. The method of claim 30, wherein the defined percentage is at least 50% of mass.

32. The method of claim 27, wherein defining gene modules comprises partitioning genes based on correlated gene expression across cells and clusters.

33. The method of claim 32, wherein partitioning comprises partitioning cells based on graph clustering.

34. The method of claim 33, wherein graph clustering further comprises dimensionality reduction using diffusion maps.

35. The method of claim 27, wherein the visualization of the developmental landscape comprises high-dimensional gene expression data in two dimensions.

36. The method of claim 33, wherein the visualization is generated using force-directed layout embedding (FLE).

37. The method of claim 27, wherein the visualization provides one or more cell types, cell ancestors, cell descendants, cell trajectories, gene modules, and cell clusters from the single cell sequencing data.

38. A computer program product, comprising:
a non-transitory computer-executable storage device having computer-readable program instructions embodied thereon that when executed by a computer cause the computer to execute the methods of anyone of claims 27 to 37.

39. A system comprising:
a storage device; and
a processor communicatively coupled to the storage device, wherein the processor executes application code instructions that are stored in the storage device and that cause the system to executed the methods of any one of claims 27 to 37.

40. A method of producing an induced pluripotent stem cell comprising introducing a nucleic acid encoding Gdf9 into a target cell to produce an induced pluripotent stem cell.
